(19) United States
(12) Patent Application Publication
(10) Pub. No.: US 2026/0085073 A1
(43) Pub. Date: Mar. 26, 2026

(54) PYRAZOLYLCARBOXAMIDE COMPOUNDS AND THEIR USE IN THERAPY

(71) Applicant: Hotspot Therapeutics, Inc., Boston, MA (US)

(72) Inventors: Kenneth G. Carson, Princeton, NJ (US); Geraldine Cirillo Harriman, Charlestown, RI (US); Arwel Lewis, Royston (GB); Gabriele Fumagalli, Cambridge (GB); Benjamin Paul Whittaker, Potton (GB); Alison Jones, Cottenham (GB); Robert Stuart Laurie Chapman, Saffron Walden (GB); Maria Angel Palomero-Vazquez, Whittlesford (GB); Riccardo Guareschi, Cambridge (GB); Christopher James Lock, Burwell (GB); James Henry Lewis, Saffron Walden (GB); Antonio Dore, Cambridge (GB); Alicia Jayne Carey, Cambridge (GB)

(21) Appl. No.: 19/110,200

(22) PCT Filed: Sep. 12, 2023
(86) PCT No.: PCT/US2023/073908

§ 371 Date: Mar. 10, 2025

Related U.S. Application Data
(60) Provisional application No. 63/405,668, filed on Sep. 12, 2022.

(51) Int. Cl. C07D 495/04; A61K 31/4155; A61K 31/4439; A61K 31/444; A61K 31/4545; A61K 31/4709; A61K 31/4725; A61K 31/4745; A61K 31/498; A61K 31/4985; A61K 31/502; A61K 31/5025; A61K 31/517; A61K 31/519; A61K 31/5365; A61K 31/538; A61K 31/5383; A61K 31/551; C07B 59/00; C07D 401/12; C07D 401/14; C07D 403/12; C07D 405/14; C07D 409/14; C07D 413/14; C07D 417/14; C07D 471/04; C07D 487/04; C07D 491/044; C07D 491/048; C07D 498/04; C07D 513/04

(52) U.S. Cl. CPC C07D 495/04; A61K 31/4155; A61K 31/4439; A61K 31/444; A61K 31/4545; A61K 31/4709; A61K 31/4725; A61K 31/4745; A61K 31/498; A61K 31/4985; A61K 31/502; A61K 31/5025; A61K 31/517; A61K 31/519; A61K 31/5365; A61K 31/538; A61K 31/5383; A61K 31/551; C07B 59/002; C07D 401/12; C07D 401/14; C07D 403/12; C07D 405/14; C07D 409/14; C07D 413/14; C07D 417/14; C07D 471/04; C07D 487/04; C07D 491/044; C07D 491/048; C07D 498/04; C07D 513/04

(57) ABSTRACT

The invention provides pyrazolylcarboxamide compounds, pharmaceutical compositions, their use for inhibiting mucosa-associated lymphoid tissue lymphoma translocation protein I (MALT1), and their use in the treatment of a disease or condition, such as a proliferative disorder, inflammatory disorder, or autoimmune disorder.

### CROSS-REFERENCE TO RELATED APPLICATIONS

This application claims the benefit of and priority to U.S. Provisional Patent Application Ser. No. 63/405,668, filed Sep. 12, 2022, the contents of which are hereby incorporated by reference in their entirety.

### FIELD OF THE INVENTION

The invention provides pyrazolylcarboxamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Current treatment options for these cancers are not effective for all patients and/or can have substantial adverse side effects. Moreover, new therapies that achieve an anti-cancer effect through a different mechanism present an opportunity to treat cancers more effectively and/or to treat cancers that have become resistant to currently available medicines.

Inflammatory disorders impact a substantial number of patients and often involve situations where the patient's biological response to a stimulus results in the immune system attacking the body's own cells or tissues. This can lead to abnormal inflammation and result in chronic pain, redness, swelling, stiffness, and/or damage to normal tissues. Current treatment options for these inflammatory disorders are not effective for all patients and/or can have substantial adverse side effects.

Human mucosa-associated lymphoid tissue protein 1 (MALT1) is a key regulator of immune responses and is an immune modulatory target for the treatment of autoimmune and inflammatory diseases. In addition, research indicates that MALT1 inhibition impairs immune suppressive function of regulatory T cells in a tumor microenvironment, implicating MALT1 inhibitors for boosting anti-tumor immunity in the treatment of solid cancers. See, for example, Isabel Hamp et al. in Expert Opinion on Therapeutic Patents (2021) vol. 12, pages 1079-1096.

The present invention addresses the foregoing needs and provides other related advantages.

### SUMMARY

The invention provides pyrazolylcarboxamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. In particular, one aspect of the invention provides a collection of pyrazolylcarboxamide compounds, such as a compound represented by Formula I:

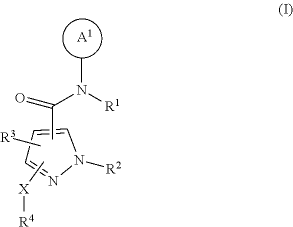

or a pharmaceutically acceptable salt thereof, where the variables are as defined in the detailed description. Further description of additional collections of pyrazolylcarboxamide compounds are described in the detailed description. The compounds may be part of a pharmaceutical composition comprising a pharmaceutically acceptable carrier.

Another aspect of the invention provides a collection of pyrazolylcarboxamide compounds, such as a compound represented by Formula II:

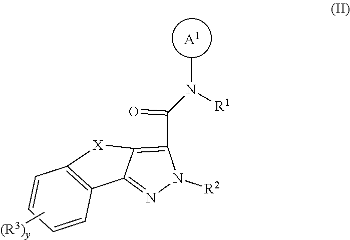

or a pharmaceutically acceptable salt thereof, where the variables are as defined in the detailed description. Further description of additional collections of pyrazolylcarboxamide compounds are described in the detailed description. The compounds may be part of a pharmaceutical composition comprising a pharmaceutically acceptable carrier.

Another aspect of the invention provides a collection of pyrazolylcarboxamide compounds, such as a compound represented by Formula III:

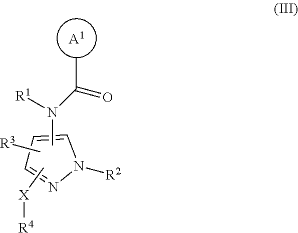

or a pharmaceutically acceptable salt thereof, where the variables are as defined in the detailed description. Further description of additional collections of pyrazolylcarboxamide compounds are described in the detailed description. The compounds may be part of a pharmaceutical composition comprising a pharmaceutically acceptable carrier.

Another aspect of the invention provides a method of treating a disease or condition mediated by MALT1 in a subject. The method comprises administering a therapeutically effective amount of a compound described herein, such as a compound of Formula I, I-1, II, or III, or other compounds in Section I of the detailed description, to a subject in need thereof to treat the disease or condition, as further described in the detailed description.

Another aspect of the invention provides a method of inhibiting the activity of MALT1. The method comprises contacting a MALT1 with an effective amount of a compound described herein, such as a compound of Formula I, I-1, II, or III, or other compounds in Section I of the detailed description, to inhibit the activity of said MALT1, as further described in the detailed description

### DETAILED DESCRIPTION

The invention provides pyrazolylcarboxamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. The practice of the present invention employs, unless otherwise indicated, conventional techniques of organic chemistry, pharmacology, molecular biology (including recombinant techniques), cell biology, biochemistry, and immunology. Such techniques are explained in the literature, such as in Comprehensive Organic Synthesis (B. M. Trost & I. Fleming, eds., 1991-1992); Handbook of Experimental Immunology (D. M. Weir & C. C. Blackwell, eds.); Current Protocols in Molecular Biology (F. M. Ausubel et al., eds., 1987, and periodic updates); and Current Protocols in Immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms in such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

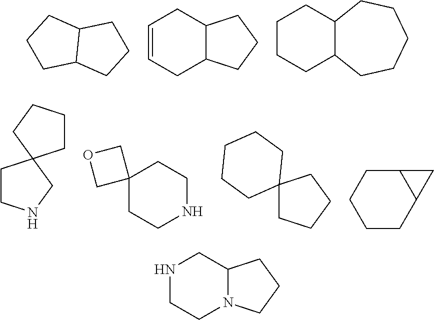

Exemplary bridged bicyclics include:

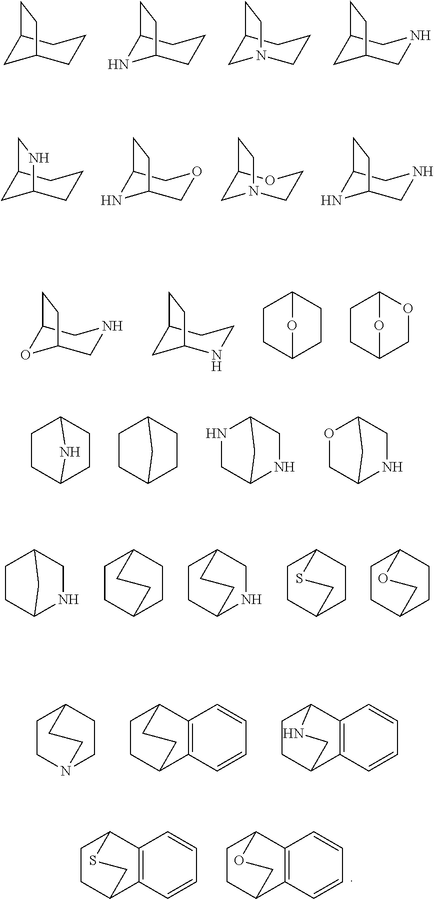

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including any oxidized form of nitrogen, sulfur, phosphorus, or silicon; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” or “halo” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

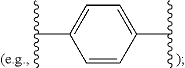 [SMILES: *c1ccc(*)cc1]

“phenylene” is a trivalent phenyl group when it has three groups attached to it

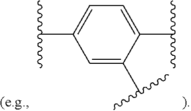 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it

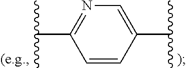 [SMILES: *c1ccc(*)nc1]

“pyridinylene” is a trivalent pyridine radical when it has three groups attached to it

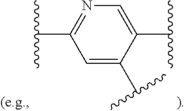 [SMILES: *c1cc(*)c(*)cn1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}2; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘}S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched) alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O) OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O) OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O) OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences 1977, 66 (1), 1-19; P. Gould, International J. of Pharmaceutics 1986, 33, 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The terms “alkenyl” and “alkynyl” are art-recognized and refer to unsaturated aliphatic groups analogous in length and possible substitution to the alkyls described above, but that contain at least one double or triple bond, respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, include humans.

As used herein, the term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate-buffered saline solution, water, emulsions (e.g., such as oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Pyrazolylcarboxamide Compounds

The invention provides pyrazolylcarboxamide compounds. The compounds may be used in the pharmaceutical compositions and therapeutic methods described herein. Exemplary compounds are described in the following sections, along with exemplary procedures for making the compounds.

One aspect of the invention provides a compound represented by Formula I:

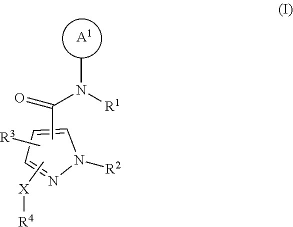

or a pharmaceutically acceptable salt thereof; wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered saturated or partially unsaturated oxo-substituted ring containing 0, 1, 2, or 3 heteroatoms independently selected from oxygen, nitrogen, and sulfur;
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, 3, or 4 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

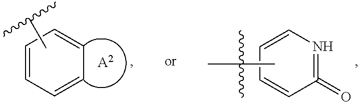

wherein the phenyl, heteroaryl,

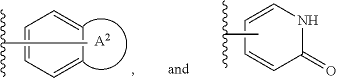

are substituted with t occurrences of R⁷;
R⁵ is —C(O)—N(R⁹)(R¹⁰) or a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹;
R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene)-, —(C₀₋₂ alkylene)-N(R⁹)—(C₀₋₂ alkylene)-, or —C(O)—; and m, n, q, t, and y are independently 0, 1, or 2;
provided that R² and R³ are not simultaneously C₁₋₄ alkyl.

The definitions of variables in Formula I above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I.

As defined generally above, A¹ is phenyl or a 6 membered heteroaryl containing 1 or 2 nitrogen atoms, wherein the phenyl and heteroaryl are substituted with m occurrences of R⁵ and n occurrences of R⁶. In certain embodiments, A¹ is pyridinyl substituted with m occurrences of R⁵ and n occurrences of R⁶. In certain embodiments, A¹ is phenyl substituted with m occurrences of R⁵ and n occurrences of R⁶. In certain embodiments, A¹ is a 6 membered heteroaryl containing 1 nitrogen atom, wherein the heteroaryl is substituted with m occurrences of R⁵ and n occurrences of R⁶. A¹ is a 6 membered heteroaryl containing 2 nitrogen atoms, wherein the heteroaryl is substituted with m occurrences of R⁵ and n occurrences of R⁶. In certain embodiments, A¹ is selected from the groups depicted in the compounds in Table 1 below.

As defined generally above, A² is a 5-7 membered partially unsaturated ring containing 0, 1, 2, or 3 heteroatoms independently selected from oxygen, nitrogen, and sulfur, or a 5-7 membered saturated or partially unsaturated oxo-substituted ring containing 0, 1, 2, or 3 heteroatoms independently selected from oxygen, nitrogen, and sulfur. In certain embodiments, A² is a 5-7 membered partially unsaturated ring containing 0, 1, 2, or 3 heteroatoms independently selected from oxygen, nitrogen, and sulfur. In certain embodiments, A² is a 5 membered partially unsaturated ring containing 0, 1, 2, or 3 heteroatoms independently selected from oxygen, nitrogen, and sulfur. In certain embodiments, A² is a 5 membered partially unsaturated ring containing 0 heteroatoms. In certain embodiments. A² is a 5 membered partially unsaturated ring containing 1 heteroatom selected from oxygen, nitrogen, and sulfur. In certain embodiments, A² is a 5 membered partially unsaturated ring containing 2 heteroatoms independently selected from oxygen, nitrogen, and sulfur. In certain embodiments, A² is a 5 membered partially unsaturated ring containing 3 heteroatoms independently selected from oxygen, nitrogen, and sulfur. In certain embodiments, A² is a 6 membered partially unsaturated ring containing 0, 1, 2, or 3 heteroatoms independently selected from oxygen, nitrogen, and sulfur. In certain embodiments, A² is a 6 membered partially unsaturated ring containing 0 heteroatoms. In certain embodiments, A² is a 6 membered partially unsaturated ring containing 1 heteroatom selected from oxygen, nitrogen, and sulfur. In certain embodiments, A² is a 6 membered partially unsaturated ring containing 2 heteroatoms independently selected from oxygen, nitrogen, and sulfur. In certain embodiments, A² is a 6 membered partially unsaturated ring containing 3 heteroatoms independently selected from oxygen, nitrogen, and sulfur. In certain embodiments, A² is a 7 membered partially unsaturated ring containing 0, 1, 2, or 3 heteroatoms independently selected from oxygen, nitrogen, and sulfur. In certain embodiments, A² is a 7 membered partially unsaturated ring containing 0 heteroatoms. In certain embodiments, A² is a 7 membered partially unsaturated ring containing 1 heteroatom selected from oxygen, nitrogen, and sulfur. In certain embodiments, A² is a 7 membered partially unsaturated ring containing 2 heteroatoms independently selected from oxygen, nitrogen, and sulfur. In certain embodiments, A² is a 7 membered partially unsaturated ring containing 3 heteroatoms independently selected from oxygen, nitrogen, and sulfur. In certain embodiments, A² is a 5-7 membered saturated or partially unsaturated oxo-substituted ring containing 0, 1, 2, or 3 heteroatoms independently selected from oxygen, nitrogen, and sulfur. In certain embodiments, A² is a 5 membered saturated or partially unsaturated oxo-substituted ring containing 0, 1, 2, or 3 heteroatoms independently selected from oxygen, nitrogen, and sulfur. In certain embodiments, A² is a 5 membered saturated or partially unsaturated oxo-substituted ring containing 0 heteroatoms. In certain embodiments, A² is a 5 membered saturated or partially unsaturated oxo-substituted ring containing 1 heteroatom selected from oxygen, nitrogen, and sulfur. In certain embodiments, A² is a 5 membered saturated or partially unsaturated oxo-substituted ring containing 2 heteroatoms independently selected from oxygen, nitrogen, and sulfur. In certain embodiments, A² is a 5 membered saturated or partially unsaturated oxo-substituted ring containing 3 heteroatoms independently selected from oxygen, nitrogen, and sulfur. In certain embodiments, A² is a 6 membered saturated or partially unsaturated oxo-substituted ring containing 0, 1, 2, or 3 heteroatoms independently selected from oxygen, nitrogen, and sulfur. In certain embodiments, A² is a 6 membered saturated or partially unsaturated oxo-substituted ring containing 0 heteroatoms. In certain embodiments, A² is a 6 membered saturated or partially unsaturated oxo-substituted ring containing 1 heteroatom selected from oxygen, nitrogen, and sulfur. In certain embodiments, A² is a 6 membered saturated or partially unsaturated oxo-substituted ring containing 2 heteroatoms independently selected from oxygen, nitrogen, and sulfur. In certain embodiments, A² is a 6 membered saturated or partially unsaturated oxo-substituted ring containing 3 heteroatoms independently selected from oxygen, nitrogen, and sulfur. In certain embodiments, A² is a 7 membered saturated or partially unsaturated oxo-substituted ring containing 0, 1, 2, or 3 heteroatoms independently selected from oxygen, nitrogen, and sulfur. In certain embodiments, A² is a 7 membered saturated or partially unsaturated oxo-substituted ring containing 0 heteroatoms. In certain embodiments, A² is a 7 membered saturated or partially unsaturated oxo-substituted ring containing 1 heteroatom selected from oxygen, nitrogen, and sulfur. In certain embodiments, A² is a 7 membered saturated or partially unsaturated oxo-substituted ring containing 2 heteroatoms independently selected from oxygen, nitrogen, and sulfur. In certain embodiments, A² is a 7 membered saturated or partially unsaturated oxo-substituted ring containing 3 heteroatoms independently selected from oxygen, nitrogen, and sulfur. In certain embodiments, A² is selected from the groups depicted in the compounds in Table 1 below.

As defined generally above, A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 4 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 4 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 4 membered saturated heterocyclyl containing 2 heteroatoms independently selected from oxygen, nitrogen, and sulfur, wherein the heterocyclyl is substituted with y occurrences of R¹². In certain embodiments, A³ represents independently for each occurrence a 5 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 5 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 5 membered saturated heterocyclyl containing 2 heteroatoms independently selected from oxygen, nitrogen, and sulfur, wherein the heterocyclyl is substituted with y occurrences of R¹². In certain embodiments, A³ represents independently for each occurrence a 6 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 6 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 6 membered saturated heterocyclyl containing 2 heteroatoms independently selected from oxygen, nitrogen, and sulfur, wherein the heterocyclyl is substituted with y occurrences of R¹². In certain embodiments, A³ is selected from the groups depicted in the compounds in Table 1 below.

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 1 below.

As defined generally above, R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms: or R² and one occurrence of R⁷ are taken together with their intervening atoms to form a 5-7 membered ring containing 1 nitrogen atom. In certain embodiments, R² is C₁₋₆ alkyl. In certain embodiments, R² is methyl. In certain embodiments, R² is C₁₋₄ haloalkyl. In certain embodiments, R² is C₁₋₆ deuteroalkyl. In certain embodiments, R² is C₁₋₆ hydroxyalkyl. In certain embodiments, R² is C₃₋₇ cycloalkyl. In certain embodiments, R² is —(C₁₋₆ alkylene)-N(R⁸)(R⁹). In certain embodiments, R² is —(C₁₋₆ alkylene)-C₁₋₆ alkoxyl. In certain embodiments, R² is a 3-5 membered saturated heterocyclyl containing 1 or 2 heteroatoms independently selected from oxygen and nitrogen. In certain embodiments, R² is a 3 membered saturated heterocyclyl containing 1 or 2 heteroatoms independently selected from oxygen and nitrogen. In certain embodiments, R² is a 3 membered saturated heterocyclyl containing 1 heteroatom selected from oxygen and nitrogen. In certain embodiments, R² is a 3 membered saturated heterocyclyl containing 2 heteroatoms independently selected from oxygen and nitrogen. In certain embodiments, R² is a 4 membered saturated heterocyclyl containing 1 or 2 heteroatoms independently selected from oxygen and nitrogen. In certain embodiments, R² is a 4 membered saturated heterocyclyl containing 1 heteroatom selected from oxygen and nitrogen. In certain embodiments, R² is a 4 membered saturated heterocyclyl containing 2 heteroatoms independently selected from oxygen and nitrogen. In certain embodiments, R² is a 5 membered saturated heterocyclyl containing 1 or 2 heteroatoms independently selected from oxygen and nitrogen. In certain embodiments, R² is a 5 membered saturated heterocyclyl containing 1 heteroatom selected from oxygen and nitrogen. In certain embodiments, R² is a 5 membered saturated heterocyclyl containing 2 heteroatoms independently selected from oxygen and nitrogen. In certain embodiments, R² and R¹ are taken together with their intervening atoms to form a 5-7 membered ring containing 2 nitrogen atoms. In certain embodiments, R² and R¹ are taken together with their intervening atoms to form a 5 membered ring containing 2 nitrogen atoms. In certain embodiments, R² and R¹ are taken together with their intervening atoms to form a 6 membered ring containing 2 nitrogen atoms. In certain embodiments, R² and R¹ are taken together with their intervening atoms to form a 7 membered ring containing 2 nitrogen atoms. In certain embodiments, R² and one occurrence of R⁷ are taken together with their intervening atoms to form a 5-7 membered ring containing 1 nitrogen atom. In certain embodiments, R² and one occurrence of R⁷ are taken together with their intervening atoms to form a 5 membered ring containing 1 nitrogen atom. In certain embodiments, R² and one occurrence of R⁷ are taken together with their intervening atoms to form a 6 membered ring containing 1 nitrogen atom. In certain embodiments, R² and one occurrence of R⁷ are taken together with their intervening atoms to form a 7 membered ring containing 1 nitrogen atom. In certain embodiments, R² is selected from the groups depicted in the compounds in Table 1 below.

As defined generally above, R³ is C₁₋₆ haloalkyl, C₁₋₆ alkyl, or hydrogen. In certain embodiments, R³ is C₁₋₆ haloalkyl. In certain embodiments, R³ is —CF₃. In certain embodiments, R³ is C₁₋₆ alkyl. In certain embodiments, R³ is methyl. In certain embodiments, R³ is C₂ alkyl. In certain embodiments, R³ is C₃ alkyl. In certain embodiments, R³ is C₄ alkyl. In certain embodiments, R³ is C₅ alkyl. In certain embodiments, R³ is C₆ alkyl. In certain embodiments, R³ is hydrogen. In certain embodiments, R³ is selected from the groups depicted in the compounds in Table 1 below.

As defined generally above, R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, 3, or 4 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

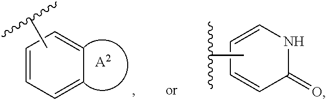

wherein the phenyl, heteroaryl,

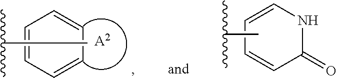

are substituted with t occurrences of R⁷. In certain embodiments, R⁴ is phenyl substituted with t occurrences of R⁷. In certain embodiments, R⁴ is a 5-6 membered heteroaryl containing 1, 2, or 3 heteroatoms independently selected from oxygen, nitrogen, and sulfur, wherein the heteroaryl is substituted with t occurrences of R⁷. In certain embodiments, R⁴ is pyridinyl substituted with t occurrences of R⁷. In certain embodiments, R⁴ is a 9-10 membered bicyclic heteroaryl containing 1, 2, 3, or 4 heteroatoms independently selected from oxygen, nitrogen, and sulfur, wherein the heteroaryl is substituted with t occurrences of R⁷. In certain embodiments, R⁴ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is

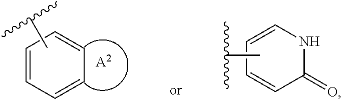

each of which are substituted with t occurrences of R⁷. In certain embodiments, R⁴ is a 5 membered heteroaryl containing 1, 2, or 3 heteroatoms independently selected from oxygen, nitrogen, and sulfur, wherein the heteroaryl is substituted with t occurrences of R⁷. In certain embodiments, R⁴ is a 5 membered heteroaryl containing 1 heteroatom selected from oxygen, nitrogen, and sulfur, wherein the heteroaryl is substituted with t occurrences of R⁷. In certain embodiments, R⁴ is a 5 membered heteroaryl containing 2 heteroatoms independently selected from oxygen, nitrogen, and sulfur, wherein the heteroaryl is substituted with t occurrences of R⁷. In certain embodiments, R⁴ is a 5 membered heteroaryl containing 3 heteroatoms independently selected from oxygen, nitrogen, and sulfur, wherein the heteroaryl is substituted with t occurrences of R⁷. In certain embodiments, R⁴ is a 6 membered heteroaryl containing 1, 2, or 3 heteroatoms independently selected from oxygen, nitrogen, and sulfur, wherein the heteroaryl is substituted with t occurrences of R⁷. In certain embodiments, R⁴ is a 6 membered heteroaryl containing 1 heteroatom selected from oxygen, nitrogen, and sulfur, wherein the heteroaryl is substituted with t occurrences of R⁷. In certain embodiments, R⁴ is a 6 membered heteroaryl containing 2 heteroatoms independently selected from oxygen, nitrogen, and sulfur, wherein the heteroaryl is substituted with t occurrences of R⁷. In certain embodiments, R⁴ is a 6 membered heteroaryl containing 3 heteroatoms independently selected from oxygen, nitrogen, and sulfur, wherein the heteroaryl is substituted with t occurrences of R⁷. In certain embodiments, R⁴ is a 9 membered bicyclic heteroaryl containing 1, 2, 3, or 4 heteroatoms independently selected from oxygen, nitrogen, and sulfur, wherein the heteroaryl is substituted with t occurrences of R⁷. In certain embodiments, R⁴ is a 9 membered bicyclic heteroaryl containing 1 heteroatom independently selected from oxygen, nitrogen, and sulfur, wherein the heteroaryl is substituted with t occurrences of R⁷. In certain embodiments, R⁴ is a 9 membered bicyclic heteroaryl containing 2 heteroatoms independently selected from oxygen, nitrogen, and sulfur, wherein the heteroaryl is substituted with t occurrences of R⁷. In certain embodiments, R⁴ is a 9 membered bicyclic heteroaryl containing 3 heteroatoms independently selected from oxygen, nitrogen, and sulfur, wherein the heteroaryl is substituted with t occurrences of R⁷. In certain embodiments, R⁴ is a 9 membered bicyclic heteroaryl containing 4 heteroatoms independently selected from oxygen, nitrogen, and sulfur, wherein the heteroaryl is substituted with t occurrences of R⁷. In certain embodiments, R⁴ is a 10 membered bicyclic heteroaryl containing 1, 2, 3, or 4 heteroatoms independently selected from oxygen, nitrogen, and sulfur, wherein the heteroaryl is substituted with t occurrences of R⁷. In certain embodiments, R⁴ is a 10 membered bicyclic heteroaryl containing 1 heteroatom independently selected from oxygen, nitrogen, and sulfur, wherein the heteroaryl is substituted with t occurrences of R⁷. In certain embodiments, R⁴ is a 10 membered bicyclic heteroaryl containing 2 heteroatoms independently selected from oxygen, nitrogen, and sulfur, wherein the heteroaryl is substituted with t occurrences of R⁷. In certain embodiments, R⁴ is a 10 membered bicyclic heteroaryl containing 3 heteroatoms independently selected from oxygen, nitrogen, and sulfur, wherein the heteroaryl is substituted with t occurrences of R⁷. In certain embodiments, R⁴ is a 10 membered bicyclic heteroaryl containing 4 heteroatoms independently selected from oxygen, nitrogen, and sulfur, wherein the heteroaryl is substituted with t occurrences of R⁷. In certain embodiments, R⁴ is a 3 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 8 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is

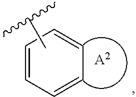

which is substituted with t occurrences of R⁷. In certain embodiments, R⁴ is

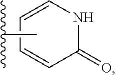

which is substituted with t occurrences of R⁷. In certain embodiments, R⁴ is selected from the groups depicted in the compounds in Table 1 below.

As defined generally above, R⁵ is —C(O)—N(R⁹)(R¹⁰) or a 5-6 membered heteroaryl containing 1, 2, or 3 heteroatoms independently selected from oxygen, nitrogen, and sulfur, wherein the heteroaryl is substituted with q occurrences of R¹². In certain embodiments, R⁵ is —C(O)—N(R⁹)(R¹⁰). In certain embodiments, R⁵ is —C(O)—N(R⁹)(R¹⁰), wherein R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁵ is —C(O)—N(R⁹)(R¹⁰), wherein R⁹ and R¹⁰ are taken together with the nitrogen atom to which they are attached to form a 4 membered heterocyclic ring containing 1 nitrogen atom, wherein the heterocyclic ring is substituted with 1 hydroxyl group. In certain embodiments, R⁵ is a 5 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 1,2,3-triazolyl, pyrazolyl, oxazolyl, imidazolyl, isoxazolyl, pyrrolyl, or furanyl, each of which substituted with q occurrences of R¹². In certain embodiments, R⁵ is a 1,2,3-triazolyl substituted with q occurrences of R¹². In certain embodiments, R⁵ is a 5 membered heteroaryl containing 1 heteroatom selected from oxygen, nitrogen, and sulfur, wherein the heteroaryl is substituted with q occurrences of R¹². In certain embodiments, R⁵ is a 5 membered heteroaryl containing 2 heteroatoms independently selected from oxygen, nitrogen, and sulfur, wherein the heteroaryl is substituted with q occurrences of R¹². In certain embodiments, R⁵ is a 5 membered heteroaryl containing 3 heteroatoms independently selected from oxygen, nitrogen, and sulfur, wherein the heteroaryl is substituted with q occurrences of R¹². In certain embodiments, R⁵ is a 6 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 6 membered heteroaryl containing 1 heteroatom selected from oxygen, nitrogen, and sulfur, wherein the heteroaryl is substituted with q occurrences of R¹². In certain embodiments, R⁵ is a 6 membered heteroaryl containing 2 heteroatoms independently selected from oxygen, nitrogen, and sulfur, wherein the heteroaryl is substituted with q occurrences of R¹². In certain embodiments, R⁵ is a 6 membered heteroaryl containing 3 heteroatoms independently selected from oxygen, nitrogen, and sulfur, wherein the heteroaryl is substituted with q occurrences of R¹². In certain embodiments, R⁵ is a pyrazolyl substituted with q occurrences of R¹². In certain embodiments, R⁵ is an oxazolyl substituted with q occurrences of R¹². In certain embodiments, R⁵ is an imidazolyl substituted with q occurrences of R¹². In certain embodiments, R⁵ is an isoxazolyl substituted with q occurrences of R¹². In certain embodiments, R⁵ is a pyrrolyl substituted with q occurrences of R¹². In certain embodiments, R⁵ is a furanyl substituted with q occurrences of R¹². In certain embodiments, R⁵ is selected from the groups depicted in the compounds in Table 1 below.

As defined generally above, R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹. In certain embodiments, R⁶ represents independently for each occurrence halo, C₁₋₆ alkyl, or C₁₋₆ haloalkyl. In certain embodiments, R⁶ is chloro. In certain embodiments, R⁶ is C₁₋₆ haloalkyl. In certain embodiments, R⁶ is —CF₃. In certain embodiments, R⁶ is halo. In certain embodiments, R⁶ is F. In certain embodiments, R⁶ is Br. In certain embodiments, R⁶ is I. In certain embodiments, R⁶ is hydroxyl. In certain embodiments, R⁶ is C₁₋₆ alkyl. In certain embodiments, R⁶ is C₁ alkyl. In certain embodiments, R⁶ is C₂ alkyl. In certain embodiments, R⁶ is C₃ alkyl. In certain embodiments, R⁶ is C₄ alkyl. In certain embodiments, R⁶ is C₅ alkyl. In certain embodiments, R⁶ is C₆ alkyl. In certain embodiments, R⁶ is C₁₋₆ hydroxyalkyl. In certain embodiments, R⁶ is C₁₋₆ alkoxyl. In certain embodiments, R⁶ is C₃₋₇ cycloalkyl. In certain embodiments, R⁶ is cyano. In certain embodiments, R⁶ is —O—C₃₋₇ cycloalkyl. In certain embodiments, R⁶ is —N(R⁹)(R¹⁰). In certain embodiments, R⁶ is —(C₀₋₄ alkylene)-C(O)R⁸. In certain embodiments, R⁶ is —C(O)N(R⁹)(R¹⁰). In certain embodiments, R⁶ is —N(R⁹)C(O)R¹¹. In certain embodiments, R⁶ is —SO₂R¹¹. In certain embodiments, R⁶ is selected from the groups depicted in the compounds in Table 1 below.

As defined generally above, R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³. In certain embodiments, R⁷ represents independently for each occurrence halo, hydroxyl, C₁₋₆ alkyl, C₁₋₆ haloalkyl, C₁₋₆ hydroxyalkyl, C₁₋₆ alkoxyl, or C₃₋₇ cycloalkyl. In certain embodiments, R⁷ represents independently for each occurrence halo, C₁₋₆ alkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, or —N(R⁹)(R¹⁰). In certain embodiments, R⁷ represents independently for each occurrence halo, C₁₋₆ alkyl, —(C₀₋₄ alkylene)-C(O)R⁸, —C(O)N(R⁹)(R¹⁰), —N(R⁹)C(O)R¹¹, —SO₂R¹¹, or —O-A³. In certain embodiments, R⁷ is halo. In certain embodiments, R⁷ is hydroxyl. In certain embodiments, R⁷ is C₁₋₆ alkyl. In certain embodiments, R⁷ is C₁₋₆ haloalkyl. In certain embodiments, R⁷ is C₁₋₆ hydroxyalkyl. In certain embodiments, R⁷ is C₁₋₆ alkoxyl. In certain embodiments, R⁷ is C₃₋₇ cycloalkyl. In certain embodiments, R⁷ is cyano. In certain embodiments, R⁷ is —O—C₃₋₇ cycloalkyl. In certain embodiments, R⁷ is —(C₀₋₄ alkylene)-CN. In certain embodiments, R⁷ is —(C₁₋₄ alkylene)-(C₁₋₆ alkoxyl). In certain embodiments, R⁷ is C₂₋₄ alkynyl. In certain embodiments, R⁷ is —N(R⁹)(R¹⁰). In certain embodiments, R⁷ is —(C₀₋₄ alkylene)-C(O)R⁸. In certain embodiments, R⁷ is —C(O)N(R⁹)(R¹⁰). In certain embodiments, R⁷ is —N(R⁹)C(O)R¹¹. In certain embodiments, R⁷ is —SO₂R¹¹. In certain embodiments, R⁷ is —O-A³. In certain embodiments, R⁷ is selected from the groups depicted in the compounds in Table 1 below.

As defined generally above, R⁸ is —OH, —O—(C₁₋₆ alkyl), —O—C₃₋₇ cycloalkyl, or A³. In certain embodiments, R⁸ is —OH. In certain embodiments, R⁸ is —O—(C₁₋₆ alkyl). In certain embodiments, R⁸ is —O—C₃₋₇ cycloalkyl. In certain embodiments, R⁸ is A³. In certain embodiments, R⁸ is selected from the groups depicted in the compounds in Table 1 below.

As defined generally above, R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is hydrogen. In certain embodiments, R⁹ is C₁₋₆ alkyl. In certain embodiments, R¹⁰ is C₁ alkyl. In certain embodiments, R¹⁰ is C₂ alkyl. In certain embodiments, R⁹ is C₃ alkyl. In certain embodiments, R⁹ is C₄ alkyl. In certain embodiments, R⁹ is C₅ alkyl. In certain embodiments, R⁹ is C₆ alkyl. In certain embodiments, R⁹ is C₃₋₇ cycloalkyl. In certain embodiments, R⁹ is C₃ cycloalkyl. In certain embodiments, R⁹ is C₄ cycloalkyl. In certain embodiments, R⁹ is C₅ cycloalkyl. In certain embodiments, R⁹ is C₆ cycloalkyl. In certain embodiments, R¹⁰ is C₇ cycloalkyl. In certain embodiments, R¹⁰ is hydrogen. In certain embodiments, R¹⁰ is C₁₋₆ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹⁰ is C₅ alkyl. In certain embodiments, R¹⁰ is C₆ alkyl. In certain embodiments, R¹⁰ is C₃₋₇ cycloalkyl. In certain embodiments, R¹⁰ is C₃ cycloalkyl. In certain embodiments, R¹⁰ is C₄ cycloalkyl. In certain embodiments, R¹⁰ is C₅ cycloalkyl. In certain embodiments, R¹⁰ is C₆ cycloalkyl. In certain embodiments, R¹⁰ is C₇ cycloalkyl. In certain embodiments, R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 3 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 4 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 5 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 6 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is selected from the groups depicted in the compounds in Table 1 below. In certain embodiments, R¹⁰ is selected from the groups depicted in the compounds in Table 1 below.

As defined generally above, R¹¹ represents independently for each occurrence C₁₋₆ alkyl or (C₀₋₅ alkylene)-C₃₋₇ cycloalkyl. In certain embodiments, R¹¹ is C₁₋₆ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹¹ is C₅ alkyl. In certain embodiments, R¹¹ is C₆ alkyl. In certain embodiments, R¹¹ is (C₀₋₅ alkylene)-C₃₋₇ cycloalkyl. In certain embodiments, R¹¹ is selected from the groups depicted in the compounds in Table 1 below.

As defined generally above, R¹² represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹² is halo. In certain embodiments, R¹² is F. In certain embodiments, R¹² is C₁. In certain embodiments, R¹² is Br. In certain embodiments, R¹² is I. In certain embodiments, R¹² is hydroxyl. In certain embodiments, R¹² is C₁₋₆ alkyl. In certain embodiments, R¹² is C₁ alkyl. In certain embodiments, R¹² is C₂ alkyl. In certain embodiments, R¹² is C₃ alkyl. In certain embodiments, R¹² is C₄ alkyl. In certain embodiments, R¹² is C₅ alkyl. In certain embodiments, R¹² is C₆ alkyl. In certain embodiments, R¹² is C₁₋₆ haloalkyl. In certain embodiments, R¹² is C₁₋₆ alkoxyl. In certain embodiments, R¹² is C₃₋₇ cycloalkyl. In certain embodiments, R¹² is selected from the groups depicted in the compounds in Table 1 below.

As defined generally above, X is a bond, C₁₋₅ alkylene, C₃₋₅ cycloalkylene, C₂₋₄ alkenylene, —(C₀₋₂ alkylene)-O—(C₀₋₂ alkylene)-, —(C₀₋₂ alkylene)-N(R⁹)—(C₀₋₂ alkylene)-, or —C(O)—. In certain embodiments, X is a bond. In certain embodiments, X is C₁₋₅ alkylene or C₃₋₅ cycloalkylene. In certain embodiments, X is C₂₋₄ alkenylene, —(C₀₋₂ alkylene)-O—(C₀₋₂ alkylene)-, or —(C₀₋₂ alkylene)-N(R⁹)—(C₀₋₂ alkylene)-. In certain embodiments, X is C₁₋₅ alkylene. In certain embodiments, X is C₃₋₅ cycloalkylene. In certain embodiments, X is C₂₋₄ alkenylene. In certain embodiments, X is —(C₀₋₂ alkylene)-O—(C₀₋₂ alkylene)-. In certain embodiments, X is —(C₀₋₂ alkylene)-N(R⁹)—(C₀₋₂ alkylene)-. In certain embodiments, X is —C(O)—. In certain embodiments, X is selected from the groups depicted in the compounds in Table 1 below.

As defined generally above, m, n, q, t, and y are independently 0, 1, or 2. In certain embodiments, t is 1. In certain embodiments, t is 0. In certain embodiments, q is 0. In certain embodiments, m is 0. In certain embodiments, m is 1. In certain embodiments, m is 2. In certain embodiments, n is 0. In certain embodiments, n is 1. In certain embodiments, n is 2. In certain embodiments, q is 1. In certain embodiments, q is 2. In certain embodiments, t is 2. In certain embodiments, y is 0. In certain embodiments, y is 1. In certain embodiments, y is 2. In certain embodiments, m is selected from the corresponding value in the groups depicted in the compounds in Table 1 below. In certain embodiments, n is selected from the corresponding value in the groups depicted in the compounds in Table 1 below. In certain embodiments, q is selected from the corresponding value in the groups depicted in the compounds in Table 1 below. In certain embodiments, t is selected from the corresponding value in the groups depicted in the compounds in Table 1 below. In certain embodiments, y is selected from the corresponding value in the groups depicted in the compounds in Table 1 below.

The description above describes multiple embodiments relating to compounds of Formula I. The patent application specifically contemplates all combinations of the embodiments.

In certain embodiments, the compound of Formula I is further defined by Formula Ia:

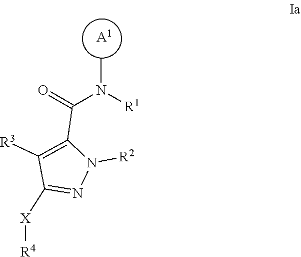

or a pharmaceutically acceptable salt thereof. In certain embodiments, the definition of variables R¹, R², R³, R⁴, A¹, and X is one of the embodiments described above in connection with Formula I.

The description above describes multiple embodiments relating to compounds of Formula Ia. The patent application specifically contemplates all combinations of the embodiments.

In certain embodiments, the compound of Formula I is further defined by Formula Ib:

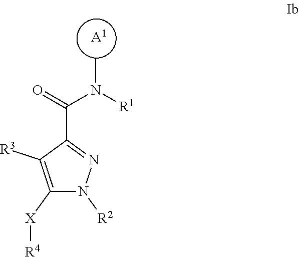

or a pharmaceutically acceptable salt thereof. In certain embodiments, the definition of variables R¹, R², R³, R⁴, A¹, and X is one of the embodiments described above in connection with Formula I.

The description above describes multiple embodiments relating to compounds of Formula Ib. The patent application specifically contemplates all combinations of the embodiments.

In certain embodiments, the compound of Formula I is further defined by Formula Ic:

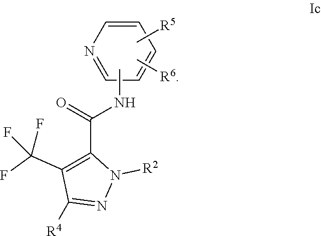

or a pharmaceutically acceptable salt thereof. In certain embodiments, the definition of variables R², R⁴, R⁵, and R⁶ is one of the embodiments described above in connection with Formula I.

The description above describes multiple embodiments relating to compounds of Formula Ic. The patent application specifically contemplates all combinations of the embodiments.

In certain embodiments, the compound of Formula I is further defined by Formula Id:

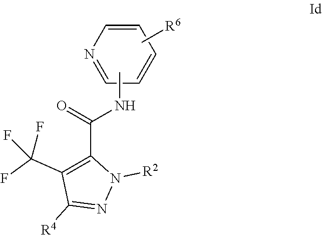

or a pharmaceutically acceptable salt thereof. In certain embodiments, the definition of variables R⁴, R⁴, and R⁶ is one of the embodiments described above in connection with Formula I.

The description above describes multiple embodiments relating to compounds of Formula Id. The patent application specifically contemplates all combinations of the embodiments.

In certain embodiments, the compound of Formula I is further defined by Formula Ie:

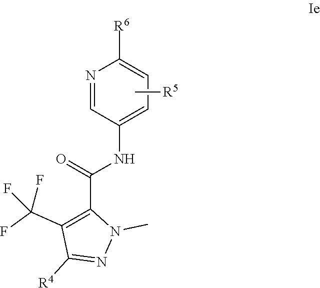

or a pharmaceutically acceptable salt thereof. In certain embodiments, the definition of variables R⁴, R⁵, and R⁶ is one of the embodiments described above in connection with Formula I.

The description above describes multiple embodiments relating to compounds of Formula Ie. The patent application specifically contemplates all combinations of the embodiments.

In certain embodiments, the compound of Formula I is further defined by Formula If:

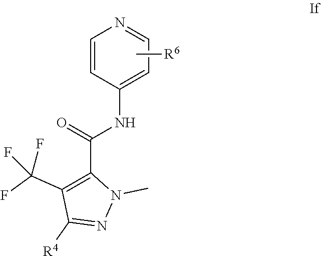

or a pharmaceutically acceptable salt thereof. In certain embodiments, the definition of variables R⁴ and R⁶ is one of the embodiments described above in connection with Formula I.

The description above describes multiple embodiments relating to compounds of Formula If. The patent application specifically contemplates all combinations of the embodiments.

In certain embodiments, the compound of Formula I is further defined by Formula Ig, Ih, Ii, or Ij:

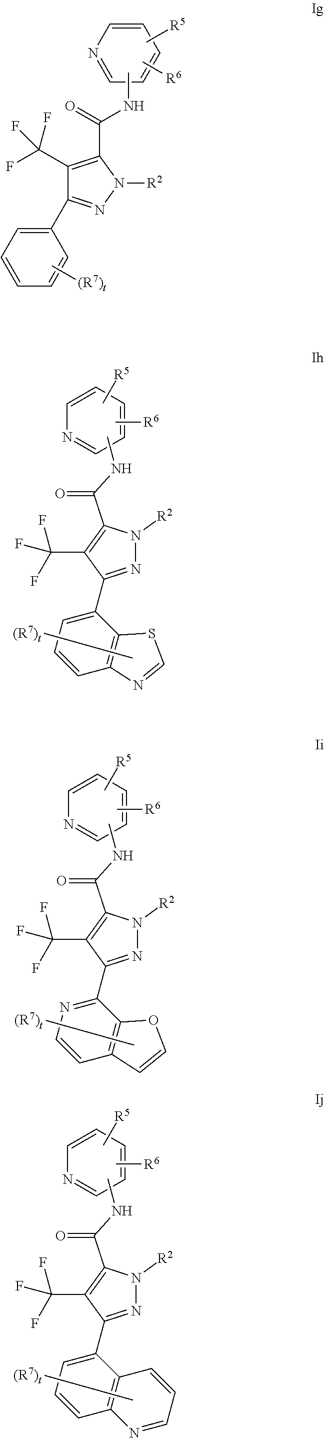

or a pharmaceutically acceptable salt thereof. In certain embodiments, the definition of variables R², R⁵, R⁶, R⁷, and t is one of the embodiments described above in connection with Formula I.

In certain embodiments, the compound of Formula I is further defined by Formula Ig or a pharmaceutically acceptable salt thereof. In certain embodiments, the compound of Formula I is further defined by Formula Ih or a pharmaceutically acceptable salt thereof. In certain embodiments, the compound of Formula I is further defined by Formula II or a pharmaceutically acceptable salt thereof. In certain embodiments, the compound of Formula I is further defined by Formula Ij or a pharmaceutically acceptable salt thereof.

The description above describes multiple embodiments relating to compounds of Formulae Ig, Ih, Ii, and Ij. The patent application specifically contemplates all combinations of the embodiments.

In certain embodiments, the compound of Formula I is further defined by Formula Ik, Il, Im, or In:

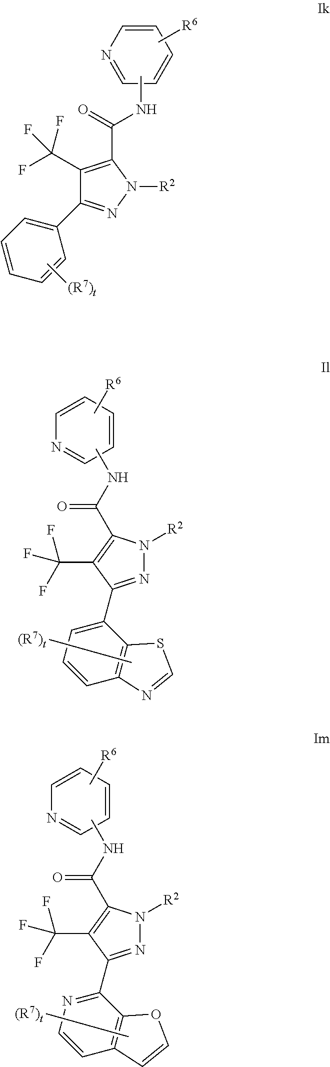

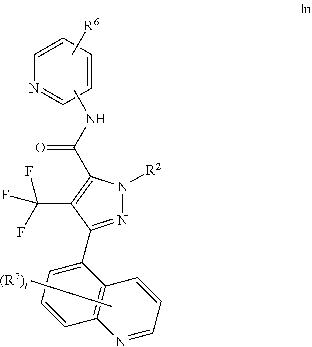

or a pharmaceutically acceptable salt thereof. In certain embodiments, the definition of variables R², R⁶, R⁷, and t is one of the embodiments described above in connection with Formula I. In certain embodiments, the compound of Formula I is further defined by Formula Ik or a pharmaceutically acceptable salt thereof. In certain embodiments, the compound of Formula I is further defined by Formula II or a pharmaceutically acceptable salt thereof. In certain embodiments, the compound of Formula I is further defined by Formula Im or a pharmaceutically acceptable salt thereof. In certain embodiments, the compound of Formula I is further defined by Formula In or a pharmaceutically acceptable salt thereof.

The description above describes multiple embodiments relating to compounds of Formulae Ik, Il, Im, and In. The patent application specifically contemplates all combinations of the embodiments.

Another aspect of the invention provides a compound represented by Formula I-1:

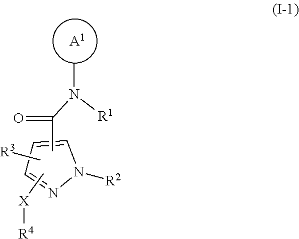

or a pharmaceutically acceptable salt thereof; wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur;
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

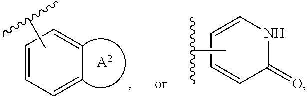

wherein the phenyl, heteroaryl,

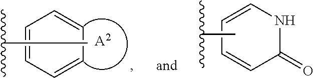

are substituted with t occurrences of R⁷;
R⁵ is a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹;
R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—; and m, n, q, t, and y are independently 0, 1, or 2;
provided that R² and R³ are not simultaneously C₁₋₄ alkyl.

The definitions of variables in Formula I-1 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-1.

As defined generally above, A¹ is phenyl or a 6 membered heteroaryl containing 1 or 2 nitrogen atoms, wherein the phenyl and heteroaryl are substituted with m occurrences of R⁵ and n occurrences of R⁶. In certain embodiments, A¹ is pyridinyl substituted with m occurrences of R⁵ and n occurrences of R⁶. In certain embodiments, A¹ is phenyl substituted with m occurrences of R⁵ and n occurrences of R⁶. In certain embodiments, A¹ is a 6 membered heteroaryl containing 1 nitrogen atom, wherein the heteroaryl is substituted with m occurrences of R⁵ and n occurrences of R⁶. A¹ is a 6 membered heteroaryl containing 2 nitrogen atoms, wherein the heteroaryl is substituted with m occurrences of R⁵ and n occurrences of R⁶. In certain embodiments, A¹ is selected from the groups depicted in the compounds in Table 1 below.

As defined generally above, A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur. In certain embodiments, A² is a 5-7 membered partially unsaturated ring containing 0, 1, 2, or 3 heteroatoms independently selected from oxygen, nitrogen, and sulfur. In certain embodiments, A² is a 5 membered partially unsaturated ring containing 0, 1, 2, or 3 heteroatoms independently selected from oxygen, nitrogen, and sulfur. In certain embodiments, A² is a 5 membered partially unsaturated ring containing 0 heteroatoms. In certain embodiments, A² is a 5 membered partially unsaturated ring containing 1 heteroatom selected from oxygen, nitrogen, and sulfur. In certain embodiments, A² is a 5 membered partially unsaturated ring containing 2 heteroatoms independently selected from oxygen, nitrogen, and sulfur. In certain embodiments, A² is a 5 membered partially unsaturated ring containing 3 heteroatoms independently selected from oxygen, nitrogen, and sulfur. In certain embodiments, A² is a 6 membered partially unsaturated ring containing 0, 1, 2, or 3 heteroatoms independently selected from oxygen, nitrogen, and sulfur. In certain embodiments, A² is a 6 membered partially unsaturated ring containing 0 heteroatoms. In certain embodiments, A² is a 6 membered partially unsaturated ring containing 1 heteroatom selected from oxygen, nitrogen, and sulfur. In certain embodiments, A² is a 6 membered partially unsaturated ring containing 2 heteroatoms independently selected from oxygen, nitrogen, and sulfur. In certain embodiments, A² is a 6 membered partially unsaturated ring containing 3 heteroatoms independently selected from oxygen, nitrogen, and sulfur. In certain embodiments, A² is a 7 membered partially unsaturated ring containing 0, 1, 2, or 3 heteroatoms independently selected from oxygen, nitrogen, and sulfur. In certain embodiments, A² is a 7 membered partially unsaturated ring containing 0 heteroatoms. In certain embodiments, A² is a 7 membered partially unsaturated ring containing 1 heteroatom selected from oxygen, nitrogen, and sulfur. In certain embodiments, A² is a 7 membered partially unsaturated ring containing 2 heteroatoms independently selected from oxygen, nitrogen, and sulfur. In certain embodiments, A² is a 7 membered partially unsaturated ring containing 3 heteroatoms independently selected from oxygen, nitrogen, and sulfur. In certain embodiments, A² is a 5-7 membered partially unsaturated oxo-substituted ring containing 0, 1, 2, or 3 heteroatoms independently selected from oxygen, nitrogen, and sulfur. In certain embodiments, A² is a 5 membered partially unsaturated oxo-substituted ring containing 0, 1, 2, or 3 heteroatoms independently selected from oxygen, nitrogen, and sulfur. In certain embodiments, A² is a 5 membered partially unsaturated oxo-substituted ring containing 0 heteroatoms. In certain embodiments, A² is a 5 membered partially unsaturated oxo-substituted ring containing 1 heteroatom selected from oxygen, nitrogen, and sulfur. In certain embodiments, A² is a 5 membered partially unsaturated oxo-substituted ring containing 2 heteroatoms independently selected from oxygen, nitrogen, and sulfur. In certain embodiments, A² is a 5 membered partially unsaturated oxo-substituted ring containing 3 heteroatoms independently selected from oxygen, nitrogen, and sulfur. In certain embodiments, A² is a 6 membered partially unsaturated oxo-substituted ring containing 0, 1, 2, or 3 heteroatoms independently selected from oxygen, nitrogen, and sulfur. In certain embodiments, A² is a 6 membered partially unsaturated oxo-substituted ring containing 0 heteroatoms. In certain embodiments, A² is a 6 membered partially unsaturated oxo-substituted ring containing 1 heteroatom selected from oxygen, nitrogen, and sulfur. In certain embodiments, A² is a 6 membered partially unsaturated oxo-substituted ring containing 2 heteroatoms independently selected from oxygen, nitrogen, and sulfur. In certain embodiments, A² is a 6 membered partially unsaturated oxo-substituted ring containing 3 heteroatoms independently selected from oxygen, nitrogen, and sulfur. In certain embodiments, A² is a 7 membered partially unsaturated oxo-substituted ring containing 0, 1, 2, or 3 heteroatoms independently selected from oxygen, nitrogen, and sulfur. In certain embodiments, A² is a 7 membered partially unsaturated oxo-substituted ring containing 0 heteroatoms. In certain embodiments, A² is a 7 membered partially unsaturated oxo-substituted ring containing 1 heteroatom selected from oxygen, nitrogen, and sulfur. In certain embodiments, A² is a 7 membered partially unsaturated oxo-substituted ring containing 2 heteroatoms independently selected from oxygen, nitrogen, and sulfur. In certain embodiments, A² is a 7 membered partially unsaturated oxo-substituted ring containing 3 heteroatoms independently selected from oxygen, nitrogen, and sulfur. In certain embodiments, A² is selected from the groups depicted in the compounds in Table 1 below.

As defined generally above, A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 4 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 4 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 4 membered saturated heterocyclyl containing 2 heteroatoms independently selected from oxygen, nitrogen, and sulfur, wherein the heterocyclyl is substituted with y occurrences of R¹². In certain embodiments, A³ represents independently for each occurrence a 5 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 5 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 5 membered saturated heterocyclyl containing 2 heteroatoms independently selected from oxygen, nitrogen, and sulfur, wherein the heterocyclyl is substituted with y occurrences of R¹². In certain embodiments, A³ represents independently for each occurrence a 6 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 6 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 6 membered saturated heterocyclyl containing 2 heteroatoms independently selected from oxygen, nitrogen, and sulfur, wherein the heterocyclyl is substituted with y occurrences of R¹². In certain embodiments, A³ is selected from the groups depicted in the compounds in Table 1 below.

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 1 below.

As defined generally above, R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom. In certain embodiments, R² is C₁₋₆ alkyl. In certain embodiments, R² is methyl. In certain embodiments, R² is C₁₋₆ haloalkyl. In certain embodiments, R² is C₁₋₆ deuteroalkyl. In certain embodiments, R² is C₁₋₆ hydroxyalkyl. In certain embodiments, R² is C₃₋₇ cycloalkyl. In certain embodiments, R² is —(C₁₋₆ alkylene)-N(R⁸)(R⁹). In certain embodiments, R² is —(C₁₋₆ alkylene)-C₁₋₆ alkoxyl. In certain embodiments, R² is a 3-5 membered saturated heterocyclyl containing 1 or 2 heteroatoms independently selected from oxygen and nitrogen. In certain embodiments, R² is a 3 membered saturated heterocyclyl containing 1 or 2 heteroatoms independently selected from oxygen and nitrogen. In certain embodiments, R² is a 3 membered saturated heterocyclyl containing 1 heteroatom selected from oxygen and nitrogen. In certain embodiments, R² is a 3 membered saturated heterocyclyl containing 2 heteroatoms independently selected from oxygen and nitrogen. In certain embodiments, R² is a 4 membered saturated heterocyclyl containing 1 or 2 heteroatoms independently selected from oxygen and nitrogen. In certain embodiments, R² is a 4 membered saturated heterocyclyl containing 1 heteroatom selected from oxygen and nitrogen. In certain embodiments, R² is a 4 membered saturated heterocyclyl containing 2 heteroatoms independently selected from oxygen and nitrogen. In certain embodiments, R² is a 5 membered saturated heterocyclyl containing 1 or 2 heteroatoms independently selected from oxygen and nitrogen. In certain embodiments, R² is a 5 membered saturated heterocyclyl containing 1 heteroatom selected from oxygen and nitrogen. In certain embodiments, R² is a 5 membered saturated heterocyclyl containing 2 heteroatoms independently selected from oxygen and nitrogen. In certain embodiments, R² and R¹ are taken together with their intervening atoms to form a 5-7 membered ring containing 2 nitrogen atoms. In certain embodiments, R² and R¹ are taken together with their intervening atoms to form a 5 membered ring containing 2 nitrogen atoms. In certain embodiments, R² and R¹ are taken together with their intervening atoms to form a 6 membered ring containing 2 nitrogen atoms. In certain embodiments, R² and R¹ are taken together with their intervening atoms to form a 7 membered ring containing 2 nitrogen atoms. In certain embodiments, R² and one occurrence of R⁷ are taken together with their intervening atoms to form a 5-7 membered ring containing 1 nitrogen atom. In certain embodiments, R² and one occurrence of R⁷ are taken together with their intervening atoms to form a 5 membered ring containing 1 nitrogen atom. In certain embodiments, R² and one occurrence of R⁷ are taken together with their intervening atoms to form a 6 membered ring containing 1 nitrogen atom. In certain embodiments, R² and one occurrence of R⁷ are taken together with their intervening atoms to form a 7 membered ring containing 1 nitrogen atom. In certain embodiments, R² is selected from the groups depicted in the compounds in Table 1 below.

As defined generally above, R³ is C₁₋₆ haloalkyl, C₁₋₆ alkyl, or hydrogen. In certain embodiments, R³ is C₁₋₆ haloalkyl. In certain embodiments, R³ is —CF₃. In certain embodiments, R³ is C₁₋₆ alkyl. In certain embodiments, R³ is methyl. In certain embodiments, R³ is C₂ alkyl. In certain embodiments, R³ is C₃ alkyl. In certain embodiments, R³ is C₄ alkyl. In certain embodiments, R³ is C₅ alkyl. In certain embodiments, R³ is C₆ alkyl. In certain embodiments, R³ is hydrogen. In certain embodiments, R³ is selected from the groups depicted in the compounds in Table 1 below.

As defined generally above, R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

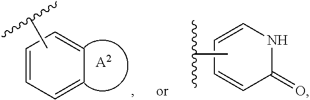

wherein the phenyl, heteroaryl,

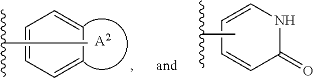

are substituted with t occurrences of R⁷. In certain embodiments, R⁴ is phenyl substituted with t occurrences of R⁷. In certain embodiments, R⁴ is a 5-6 membered heteroaryl containing 1, 2, or 3 heteroatoms independently selected from oxygen, nitrogen, and sulfur, wherein the heteroaryl is substituted with t occurrences of R⁷. In certain embodiments, R⁴ is pyridinyl substituted with t occurrences of R⁷. In certain embodiments, R⁴ is a 9-10 membered bicyclic heteroaryl containing 1, 2, or 3 heteroatoms independently selected from oxygen, nitrogen, and sulfur, wherein the heteroaryl is substituted with t occurrences of R⁷. In certain embodiments, R⁴ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is

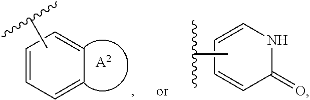

each of which are substituted with t occurrences of R⁷. In certain embodiments, R⁴ is a 5 membered heteroaryl containing 1, 2, or 3 heteroatoms independently selected from oxygen, nitrogen, and sulfur, wherein the heteroaryl is substituted with t occurrences of R⁷. In certain embodiments, R⁴ is a 5 membered heteroaryl containing 1 heteroatom selected from oxygen, nitrogen, and sulfur, wherein the heteroaryl is substituted with t occurrences of R⁷. In certain embodiments, R⁴ is a 5 membered heteroaryl containing 2 heteroatoms independently selected from oxygen, nitrogen, and sulfur, wherein the heteroaryl is substituted with t occurrences of R⁷. In certain embodiments, R⁴ is a 5 membered heteroaryl containing 3 heteroatoms independently selected from oxygen, nitrogen, and sulfur, wherein the heteroaryl is substituted with t occurrences of R⁷. In certain embodiments, R⁴ is a 6 membered heteroaryl containing 1, 2, or 3 heteroatoms independently selected from oxygen, nitrogen, and sulfur, wherein the heteroaryl is substituted with t occurrences of R⁷. In certain embodiments, R⁴ is a 6 membered heteroaryl containing 1 heteroatom selected from oxygen, nitrogen, and sulfur, wherein the heteroaryl is substituted with t occurrences of R⁷. In certain embodiments, R⁴ is a 6 membered heteroaryl containing 2 heteroatoms independently selected from oxygen, nitrogen, and sulfur, wherein the heteroaryl is substituted with t occurrences of R⁷. In certain embodiments, R⁴ is a 6 membered heteroaryl containing 3 heteroatoms independently selected from oxygen, nitrogen, and sulfur, wherein the heteroaryl is substituted with t occurrences of R⁷. In certain embodiments, R⁴ is a 9 membered bicyclic heteroaryl containing 1, 2, or 3 heteroatoms independently selected from oxygen, nitrogen, and sulfur, wherein the heteroaryl is substituted with t occurrences of R⁷. In certain embodiments, R⁴ is a 9 membered bicyclic heteroaryl containing 1 heteroatom independently selected from oxygen, nitrogen, and sulfur, wherein the heteroaryl is substituted with t occurrences of R⁷. In certain embodiments, R⁴ is a 9 membered bicyclic heteroaryl containing 2 heteroatoms independently selected from oxygen, nitrogen, and sulfur, wherein the heteroaryl is substituted with t occurrences of R⁷. In certain embodiments, R⁴ is a 9 membered bicyclic heteroaryl containing 3 heteroatoms independently selected from oxygen, nitrogen, and sulfur, wherein the heteroaryl is substituted with t occurrences of R⁷. In certain embodiments, R⁴ is a 10 membered bicyclic heteroaryl containing 1, 2, or 3 heteroatoms independently selected from oxygen, nitrogen, and sulfur, wherein the heteroaryl is substituted with t occurrences of R⁷. In certain embodiments, R⁴ is a 10 membered bicyclic heteroaryl containing 1 heteroatom independently selected from oxygen, nitrogen, and sulfur, wherein the heteroaryl is substituted with t occurrences of R⁷. In certain embodiments, R⁴ is a 10 membered bicyclic heteroaryl containing 2 heteroatoms independently selected from oxygen, nitrogen, and sulfur, wherein the heteroaryl is substituted with t occurrences of R⁷. In certain embodiments, R⁴ is a 10 membered bicyclic heteroaryl containing 3 heteroatoms independently selected from oxygen, nitrogen, and sulfur, wherein the heteroaryl is substituted with t occurrences of R⁷. In certain embodiments, R⁴ is a 3 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 8 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is

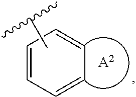

which is substituted with t occurrences of R⁷. In certain embodiments, R⁴ is

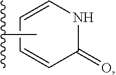

which is substituted with t occurrences of R⁷. In certain embodiments, R⁴ is selected from the groups depicted in the compounds in Table 1 below.

As defined generally above, R⁵ is a 5-6 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 5 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 1,2,3-triazolyl, pyrazolyl, oxazolyl, imidazolyl, isoxazolyl, pyrrolyl, or furanyl, each of which substituted with q occurrences of R¹². In certain embodiments, R⁵ is a 1,2,3-triazolyl substituted with q occurrences of R¹². In certain embodiments, R⁵ is a 5 membered heteroaryl containing 1 heteroatom selected from oxygen, nitrogen, and sulfur, wherein the heteroaryl is substituted with q occurrences of R¹². In certain embodiments, R⁵ is a 5 membered heteroaryl containing 2 heteroatoms independently selected from oxygen, nitrogen, and sulfur, wherein the heteroaryl is substituted with q occurrences of R¹². In certain embodiments, R⁵ is a 5 membered heteroaryl containing 3 heteroatoms independently selected from oxygen, nitrogen, and sulfur, wherein the heteroaryl is substituted with q occurrences of R¹². In certain embodiments, R⁵ is a 6 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 6 membered heteroaryl containing 1 heteroatom selected from oxygen, nitrogen, and sulfur, wherein the heteroaryl is substituted with q occurrences of R¹². In certain embodiments, R⁵ is a 6 membered heteroaryl containing 2 heteroatoms independently selected from oxygen, nitrogen, and sulfur, wherein the heteroaryl is substituted with q occurrences of R¹². In certain embodiments, R⁵ is a 6 membered heteroaryl containing 3 heteroatoms independently selected from oxygen, nitrogen, and sulfur, wherein the heteroaryl is substituted with q occurrences of R¹². In certain embodiments, R⁵ is a pyrazolyl substituted with q occurrences of R¹². In certain embodiments, R⁵ is an oxazolyl substituted with q occurrences of R¹². In certain embodiments, R⁵ is an imidazolyl substituted with q occurrences of R¹². In certain embodiments, R⁵ is an isoxazolyl substituted with q occurrences of R¹². In certain embodiments, R⁵ is a pyrrolyl substituted with q occurrences of R¹². In certain embodiments, R⁵ is a furanyl substituted with q occurrences of R¹². In certain embodiments, R⁵ is selected from the groups depicted in the compounds in Table 1 below.

As defined generally above, R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹. In certain embodiments, R⁶ represents independently for each occurrence halo, C₁₋₆ alkyl, or C₁₋₆ haloalkyl. In certain embodiments, R⁶ is chloro. In certain embodiments, R⁶ is C₁₋₆ haloalkyl.

In certain embodiments, R⁶ is —CF₃. In certain embodiments, R⁶ is halo. In certain embodiments, R⁶ is F. In certain embodiments, R⁶ is Br. In certain embodiments, R⁶ is I. In certain embodiments, R⁶ is hydroxyl. In certain embodiments, R⁶ is C₁₋₆ alkyl. In certain embodiments, R⁶ is C₁ alkyl. In certain embodiments, R⁶ is C₂ alkyl. In certain embodiments, R⁶ is C₃ alkyl. In certain embodiments, R⁶ is C₄ alkyl. In certain embodiments, R⁶ is C₅ alkyl. In certain embodiments, R⁶ is C₆ alkyl. In certain embodiments, R⁶ is C₁₋₆ hydroxyalkyl. In certain embodiments, R⁶ is C₁₋₆ alkoxyl. In certain embodiments, R⁶ is C₃₋₇ cycloalkyl. In certain embodiments, R⁶ is cyano. In certain embodiments, R⁶ is —O—C₃₋₇ cycloalkyl. In certain embodiments, R⁶ is —N(R⁹)(R¹⁰). In certain embodiments, R⁶ is —(C₀₋₄ alkylene)-C(O)R⁸. In certain embodiments, R⁶ is —C(O)N(R⁹)(R¹⁰). In certain embodiments, R⁶ is —N(R⁹)C(O)R¹¹. In certain embodiments, R⁶ is —SO₂R¹¹. In certain embodiments, R⁶ is selected from the groups depicted in the compounds in Table 1 below.

As defined generally above, R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³. In certain embodiments, R⁷ represents independently for each occurrence halo, hydroxyl, C₁₋₆ alkyl, C₁₋₆ haloalkyl, C₁₋₆ hydroxyalkyl, C₁₋₆ alkoxyl, or C₃₋₇ cycloalkyl. In certain embodiments, R⁷ represents independently for each occurrence halo, C₁₋₆ alkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, or —N(R⁹)(R¹⁰). In certain embodiments, R¹ represents independently for each occurrence halo, C₁₋₆ alkyl, —(C₀₋₄ alkylene)-C(O)R⁸, —C(O)N(R⁹)(R¹⁰), —N(R⁹)C(O)R¹¹, —SO₂R¹¹, or —O-A³. In certain embodiments, R⁷ is halo. In certain embodiments, R⁷ is hydroxyl. In certain embodiments, R⁷ is C₁₋₆ alkyl. In certain embodiments, R⁷ is C₁₋₆ haloalkyl. In certain embodiments, R⁷ is C₁₋₆ hydroxyalkyl. In certain embodiments, R⁷ is C₁₋₆ alkoxyl. In certain embodiments, R⁷ is C₃₋₇ cycloalkyl. In certain embodiments, R⁷ is cyano. In certain embodiments, R⁷ is —O—C₃₋₇ cycloalkyl. In certain embodiments, R⁷ is —(C₀₋₄ alkylene)-CN. In certain embodiments, R⁷ is —(C₁₋₄ alkylene)-(C₁₋₆ alkoxyl). In certain embodiments, R⁷ is C₂₋₄ alkynyl. In certain embodiments, R⁷ is —N(R⁹)(R¹⁰). In certain embodiments, R⁷ is —(C₀₋₄ alkylene)-C(O)R⁸. In certain embodiments, R⁷ is —C(O)N(R⁹)(R¹⁰). In certain embodiments, R⁷ is —N(R⁹)C(O)R¹¹. In certain embodiments, R⁷ is —SO₂R¹¹. In certain embodiments, R⁷ is —O-A³. In certain embodiments, R⁷ is selected from the groups depicted in the compounds in Table 1 below.

As defined generally above, R⁸ is —OH, —O—(C₁₋₆ alkyl), —O—C₃₋₇ cycloalkyl, or A³. In certain embodiments, R⁸ is —OH. In certain embodiments, R⁸ is —O—(C₁₋₆ alkyl). In certain embodiments, R⁸ is —O—C₃₋₇ cycloalkyl. In certain embodiments, R⁸ is A³. In certain embodiments, R⁸ is selected from the groups depicted in the compounds in Table 1 below.

As defined generally above, R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is hydrogen. In certain embodiments, R⁹ is C₁₋₆ alkyl. In certain embodiments, R⁹ is C₁ alkyl. In certain embodiments, R⁹ is C₂ alkyl. In certain embodiments, R⁹ is C₃ alkyl. In certain embodiments, R⁹ is C₄ alkyl. In certain embodiments, R⁹ is C₅ alkyl. In certain embodiments, R⁹ is C₆ alkyl. In certain embodiments, R⁹ is C₃₋₇ cycloalkyl. In certain embodiments, R⁹ is C₃ cycloalkyl. In certain embodiments, R⁹ is C₄ cycloalkyl. In certain embodiments, R⁹ is C₅ cycloalkyl. In certain embodiments, R⁹ is C₆ cycloalkyl. In certain embodiments, R⁹ is C₇ cycloalkyl. In certain embodiments, R¹⁰ is hydrogen. In certain embodiments, R¹⁰ is C₁₋₆ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹⁰ is C₅ alkyl. In certain embodiments, R¹⁰ is C₆ alkyl. In certain embodiments, R¹⁰ is C₃₋₇ cycloalkyl. In certain embodiments, R¹⁰ is C₃ cycloalkyl. In certain embodiments, R¹⁰ is C₄ cycloalkyl. In certain embodiments, R¹⁰ is C₅ cycloalkyl. In certain embodiments, R¹⁰ is C₆ cycloalkyl. In certain embodiments, R¹⁰ is C₇ cycloalkyl. In certain embodiments, R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 3 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 4 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 5 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 6 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is selected from the groups depicted in the compounds in Table 1 below. In certain embodiments, R¹⁰ is selected from the groups depicted in the compounds in Table 1 below.

As defined generally above, R¹¹ represents independently for each occurrence C₁₋₆ alkyl or (C₀₋₅ alkylene)-C₃₋₇ cycloalkyl. In certain embodiments, R¹¹ is C₁₋₆ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹¹ is C₅ alkyl. In certain embodiments, R¹¹ is C₆ alkyl. In certain embodiments, R¹¹ is (C₀₋₅ alkylene)-C₃₋₇ cycloalkyl. In certain embodiments, R¹¹ is selected from the groups depicted in the compounds in Table 1 below.

As defined generally above, R¹² represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹² is halo. In certain embodiments, R¹² is F. In certain embodiments, R¹² is C₁. In certain embodiments, R¹² is Br. In certain embodiments, R¹² is I. In certain embodiments, R¹² is hydroxyl. In certain embodiments, R¹² is C₁₋₆ alkyl. In certain embodiments, R¹² is C₁ alkyl. In certain embodiments, R¹² is C₂ alkyl. In certain embodiments, R¹² is C₃ alkyl. In certain embodiments, R¹² is C₄ alkyl. In certain embodiments, R¹² is C₅ alkyl. In certain embodiments, R¹² is C₆ alkyl. In certain embodiments, R¹² is C₁₋₆ haloalkyl. In certain embodiments, R¹² is C₁₋₆ alkoxyl. In certain embodiments, R¹² is C₃₋₇ cycloalkyl. In certain embodiments, R¹² is selected from the groups depicted in the compounds in Table 1 below.

As defined generally above, X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—. In certain embodiments, X is a bond. In certain embodiments, X is C₁₋₅ alkylene or C₃₋₅ cycloalkylene. In certain embodiments, X is C₂₋₄ alkenylene, —(C₀₋₂ alkylene)-O—(C₀₋₂ alkylene), or —(C₀₋₂ alkylene)-N(R⁹)—(C₀₋₂ alkylene). In certain embodiments, X is C₁₋₅ alkylene. In certain embodiments, X is C₃₋₅ cycloalkylene. In certain embodiments, X is C₂₋₄ alkenylene. In certain embodiments, X is —(C₀₋₂ alkylene)-O—(C₀₋₂ alkylene). In certain embodiments, X is —(C₀₋₂ alkylene)-N(R⁹)—(C₀₋₂ alkylene). In certain embodiments, X is —C(O)—. In certain embodiments, X is selected from the groups depicted in the compounds in Table 1 below.

As defined generally above, m, n, q, t, and y are independently 0, 1, or 2. In certain embodiments, t is 1. In certain embodiments, t is 0. In certain embodiments, q is 0. In certain embodiments, m is 0. In certain embodiments, m is 1. In certain embodiments, m is 2. In certain embodiments, n is 0. In certain embodiments, n is 1. In certain embodiments, n is 2. In certain embodiments, q is 1. In certain embodiments, q is 2. In certain embodiments, t is 2. In certain embodiments, y is 0. In certain embodiments, y is 1. In certain embodiments, y is 2. In certain embodiments, m is selected from the corresponding value in the groups depicted in the compounds in Table 1 below. In certain embodiments, n is selected from the corresponding value in the groups depicted in the compounds in Table 1 below. In certain embodiments, q is selected from the corresponding value in the groups depicted in the compounds in Table 1 below. In certain embodiments, t is selected from the corresponding value in the groups depicted in the compounds in Table 1 below. In certain embodiments, y is selected from the corresponding value in the groups depicted in the compounds in Table 1 below.

The description above describes multiple embodiments relating to compounds of Formula I-1. The patent application specifically contemplates all combinations of the embodiments.

In certain embodiments, the compound of Formula I-1 is further defined by Formula Ia-1:

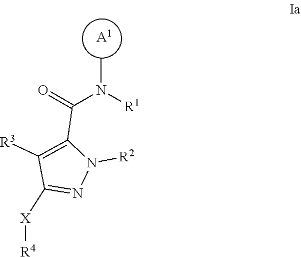

or a pharmaceutically acceptable salt thereof. In certain embodiments, the definition of variables R¹, R², R³, R⁴, A¹, and X is one of the embodiments described above in connection with Formula I-1.

The description above describes multiple embodiments relating to compounds of Formula Ia-1. The patent application specifically contemplates all combinations of the embodiments.

In certain embodiments, the compound of Formula I-1 is further defined by Formula Ib-1:

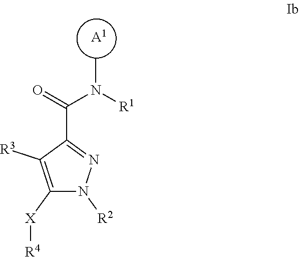

or a pharmaceutically acceptable salt thereof. In certain embodiments, the definition of variables R¹, R², R³, R⁴, A¹, and X is one of the embodiments described above in connection with Formula I-1.

The description above describes multiple embodiments relating to compounds of Formula Ib-1. The patent application specifically contemplates all combinations of the embodiments.

In certain embodiments, the compound of Formula I-1 is further defined by Formula Ic-1:

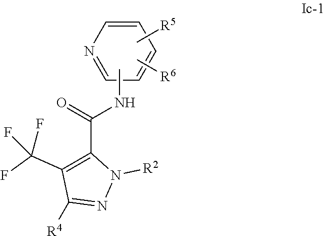

or a pharmaceutically acceptable salt thereof. In certain embodiments, the definition of variables R², R⁴, R⁵, and R⁶ is one of the embodiments described above in connection with Formula 1-1.

The description above describes multiple embodiments relating to compounds of Formula Ic-1. The patent application specifically contemplates all combinations of the embodiments.

In certain embodiments, the compound of Formula I-1 is further defined by Formula Id-1:

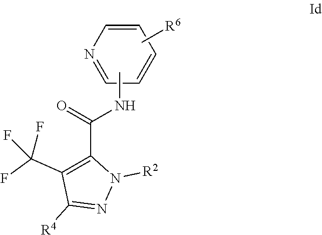

or a pharmaceutically acceptable salt thereof. In certain embodiments, the definition of variables R⁴, R⁴, and R⁶ is one of the embodiments described above in connection with Formula I-1.

The description above describes multiple embodiments relating to compounds of Formula Id-1. The patent application specifically contemplates all combinations of the embodiments.

In certain embodiments, the compound of Formula I-1 is further defined by Formula Ie-1:

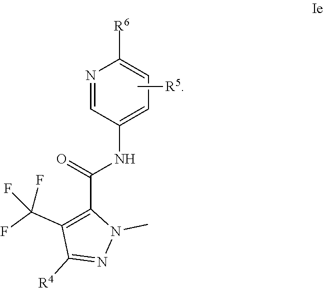

or a pharmaceutically acceptable salt thereof. In certain embodiments, the definition of variables R⁴, R⁵, and R⁶ is one of the embodiments described above in connection with Formula I-1.

The description above describes multiple embodiments relating to compounds of Formula Ie-1. The patent application specifically contemplates all combinations of the embodiments.

In certain embodiments, the compound of Formula I-1 is further defined by Formula If-1:

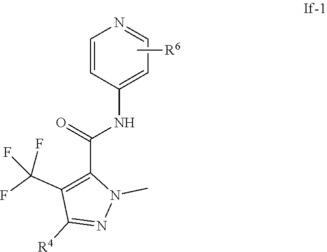

or a pharmaceutically acceptable salt thereof. In certain embodiments, the definition of variables R⁴ and R⁶ is one of the embodiments described above in connection with Formula I-1.

The description above describes multiple embodiments relating to compounds of Formula If-1. The patent application specifically contemplates all combinations of the embodiments.

In certain embodiments, the compound of Formula I-1 is further defined by Formula Ig-1, Ih-1, Ii-1, or Ij-1:

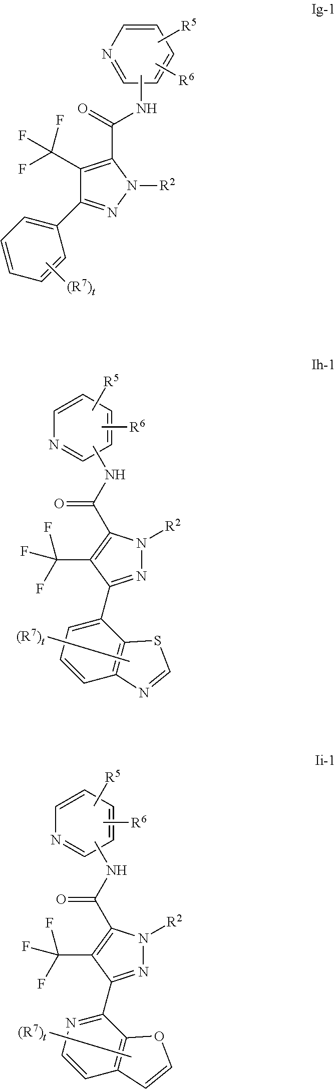

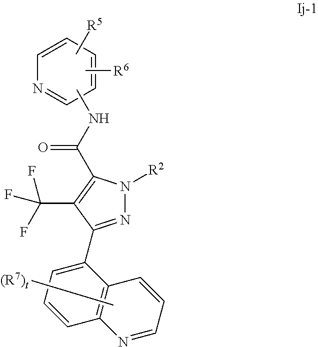

or a pharmaceutically acceptable salt thereof. In certain embodiments, the definition of variables R², R⁵, R⁶, R⁷, and t is one of the embodiments described above in connection with Formula I-1. In certain embodiments, the compound of Formula I-1 is further defined by Formula Ig-1 or a pharmaceutically acceptable salt thereof. In certain embodiments, the compound of Formula I-1 is further defined by Formula Ih-1 or a pharmaceutically acceptable salt thereof. In certain embodiments, the compound of Formula I-1 is further defined by Formula II-1 or a pharmaceutically acceptable salt thereof. In certain embodiments, the compound of Formula 1-1 is further defined by Formula Ij-1 or a pharmaceutically acceptable salt thereof.

The description above describes multiple embodiments relating to compounds of Formulae Ig-1, Ih-1, Ii-1, and Ij-1. The patent application specifically contemplates all combinations of the embodiments.

In certain embodiments, the compound of Formula I-1 is further defined by Formula Ik-1, Il-1, Im-1, or In-1:

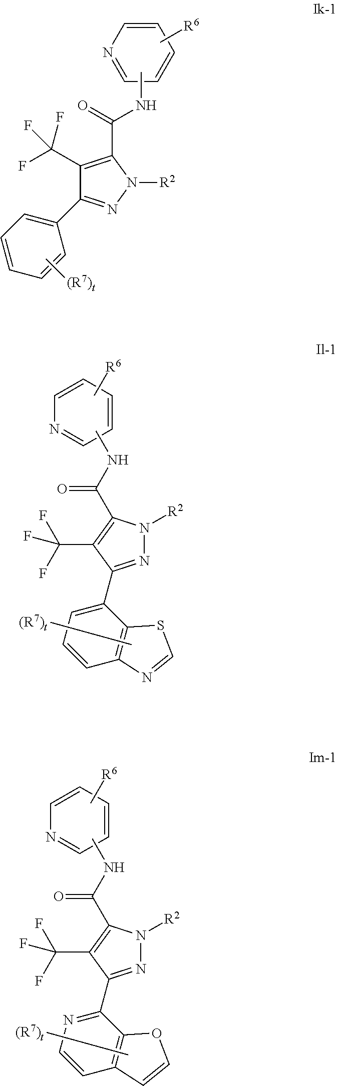

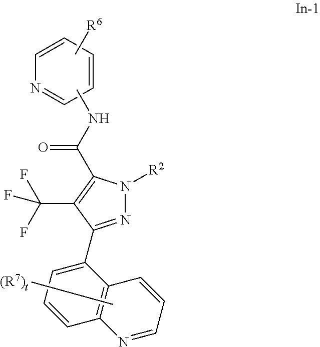

or a pharmaceutically acceptable salt thereof. In certain embodiments, the definition of variables R², R⁶, R⁷, and t is one of the embodiments described above in connection with Formula 1-1. In certain embodiments, the compound of Formula I-1 is further defined by Formula Ik-1 or a pharmaceutically acceptable salt thereof. In certain embodiments, the compound of Formula I-1 is further defined by Formula II-1 or a pharmaceutically acceptable salt thereof. In certain embodiments, the compound of Formula I-1 is further defined by Formula Im-1 or a pharmaceutically acceptable salt thereof. In certain embodiments, the compound of Formula 1-1 is further defined by Formula In-1 or a pharmaceutically acceptable salt thereof.

The description above describes multiple embodiments relating to compounds of Formulae Ik-1, Il-1, Im-1, and In-1. The patent application specifically contemplates all combinations of the embodiments.

Another aspect of the invention provides a compound in Table 1 below, or a pharmaceutically acceptable salt thereof. In certain embodiments, the compound is a compound in Table I.

TABLE 1

| Compound No. | Chemical Structure |
|---|---|
| I-1 | 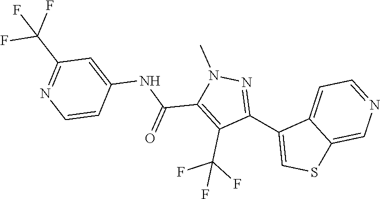 [SMILES: Cn1nc(-c2csc3cnccc23)c(C(F)(F)F)c1C(=O)Nc1ccnc(C(F)(F)F)c1] |
| I-2 | 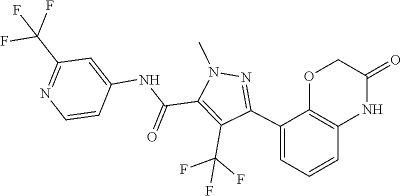 [SMILES: Cn1nc(-c2cccc3c2OCC(=O)N3)c(C(F)(F)F)c1C(=O)Nc1ccnc(C(F)(F)F)c1] |
| I-3 | 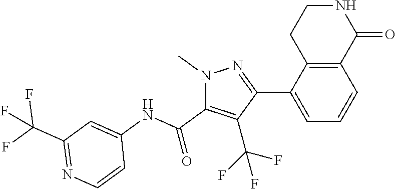 [SMILES: Cn1nc(-c2cccc3c2CCNC3=O)c(C(F)(F)F)c1C(=O)Nc1ccnc(C(F)(F)F)c1] |
| I-4 | 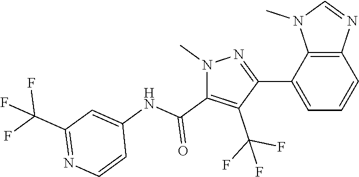 [SMILES: Cn1nc(-c2cccc3ncn(C)c23)c(C(F)(F)F)c1C(=O)Nc1ccnc(C(F)(F)F)c1] |
| I-5 | 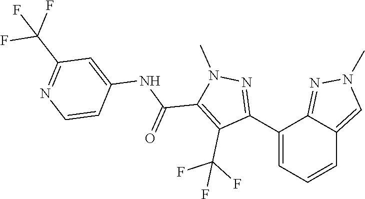 [SMILES: Cn1cc2cccc(-c3nn(C)c(C(=O)Nc4ccnc(C(F)(F)F)c4)c3C(F)(F)F)c2n1] |
| I-6 | 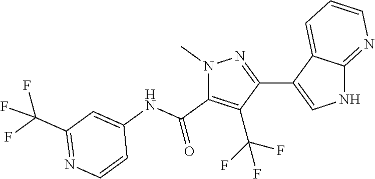 [SMILES: Cn1nc(-c2c[nH]c3ncccc23)c(C(F)(F)F)c1C(=O)Nc1ccnc(C(F)(F)F)c1] |
| I-7 | 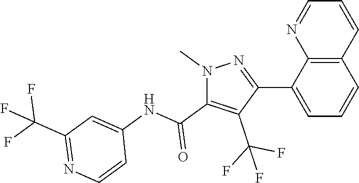 [SMILES: Cn1nc(-c2cccc3cccnc23)c(C(F)(F)F)c1C(=O)Nc1ccnc(C(F)(F)F)c1] |
| I-8 | 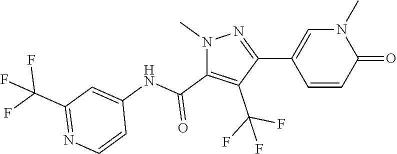 [SMILES: Cn1nc(-c2ccc(=O)n(C)c2)c(C(F)(F)F)c1C(=O)Nc1ccnc(C(F)(F)F)c1] |
| I-9 | 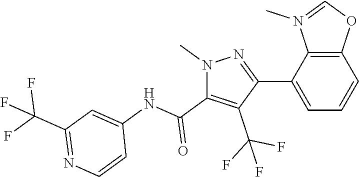 |
| I-10 | 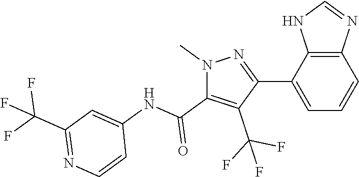 [SMILES: Cn1nc(-c2cccc3nc[nH]c23)c(C(F)(F)F)c1C(=O)Nc1ccnc(C(F)(F)F)c1] |
| I-11 | 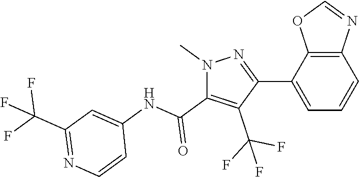 [SMILES: Cn1nc(-c2cccc3ncoc23)c(C(F)(F)F)c1C(=O)Nc1ccnc(C(F)(F)F)c1] |
| I-12 | 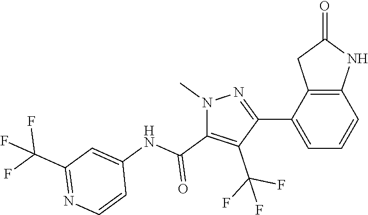 [SMILES: Cn1nc(-c2cccc3c2CC(=O)N3)c(C(F)(F)F)c1C(=O)Nc1ccnc(C(F)(F)F)c1] |
| I-13 | 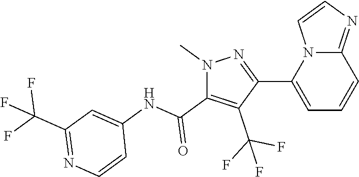 [SMILES: Cn1nc(-c2cccc3nccn23)c(C(F)(F)F)c1C(=O)Nc1ccnc(C(F)(F)F)c1] |
| I-14 | 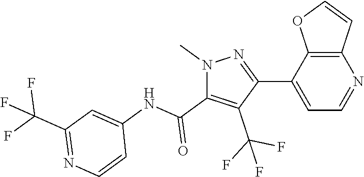 [SMILES: Cn1nc(-c2ccnc3ccoc23)c(C(F)(F)F)c1C(=O)Nc1ccnc(C(F)(F)F)c1] |
| I-15 | 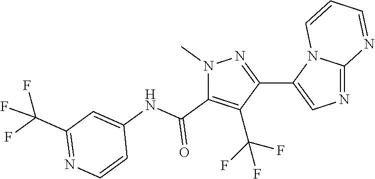 [SMILES: Cn1nc(-c2cnc3ncccn23)c(C(F)(F)F)c1C(=O)Nc1ccnc(C(F)(F)F)c1] |
| I-16 | 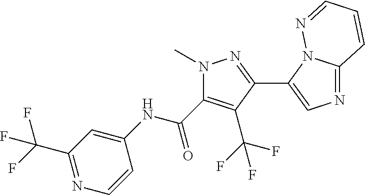 [SMILES: Cn1nc(-c2cnc3cccnn23)c(C(F)(F)F)c1C(=O)Nc1ccnc(C(F)(F)F)c1] |
| I-17 | 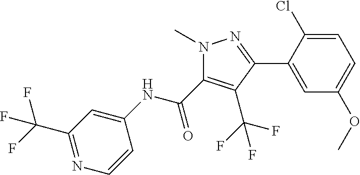 [SMILES: COc1ccc(Cl)c(-c2nn(C)c(C(=O)Nc3ccnc(C(F)(F)F)c3)c2C(F)(F)F)c1] |
| I-18 | 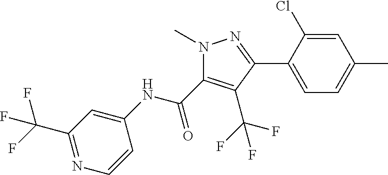 [SMILES: Cc1ccc(-c2nn(C)c(C(=O)Nc3ccnc(C(F)(F)F)c3)c2C(F)(F)F)c(Cl)c1] |
| I-19 | 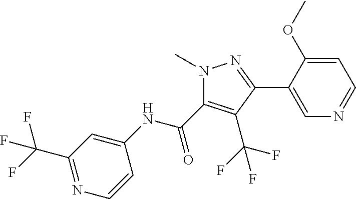 [SMILES: COc1ccncc1-c1nn(C)c(C(=O)Nc2ccnc(C(F)(F)F)c2)c1C(F)(F)F] |
| I-20 | 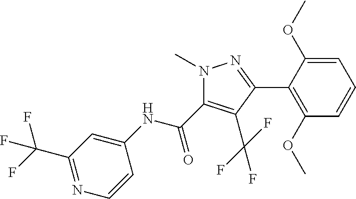 [SMILES: COc1cccc(OC)c1-c1nn(C)c(C(=O)Nc2ccnc(C(F)(F)F)c2)c1C(F)(F)F] |
| I-21 | 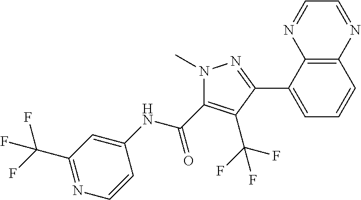 [SMILES: Cn1nc(-c2cccc3nccnc23)c(C(F)(F)F)c1C(=O)Nc1ccnc(C(F)(F)F)c1] |
| I-22 | 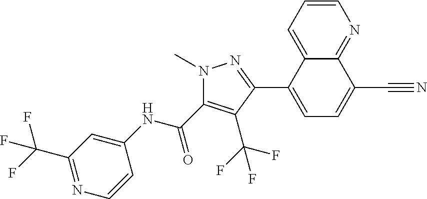 [SMILES: Cn1nc(-c2ccc(C#N)c3ncccc23)c(C(F)(F)F)c1C(=O)Nc1ccnc(C(F)(F)F)c1] |
| I-23 | 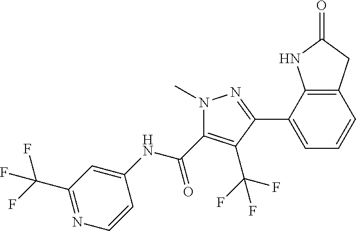 [SMILES: Cn1nc(-c2cccc3c2NC(=O)C3)c(C(F)(F)F)c1C(=O)Nc1ccnc(C(F)(F)F)c1] |
| I-24 | 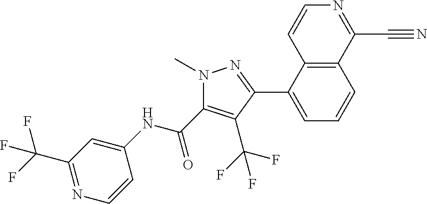 [SMILES: Cn1nc(-c2cccc3c(C#N)nccc23)c(C(F)(F)F)c1C(=O)Nc1ccnc(C(F)(F)F)c1] |
| I-25 | 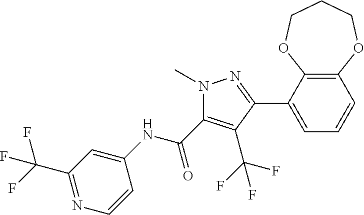 [SMILES: Cn1nc(-c2cccc3c2OCCCO3)c(C(F)(F)F)c1C(=O)Nc1ccnc(C(F)(F)F)c1] |
| I-26 | 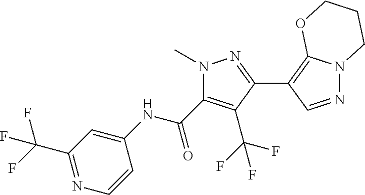 [SMILES: Cn1nc(-c2cnn3c2OCCC3)c(C(F)(F)F)c1C(=O)Nc1ccnc(C(F)(F)F)c1] |
| I-27 | 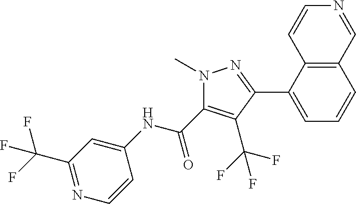 [SMILES: Cn1nc(-c2cccc3cnccc23)c(C(F)(F)F)c1C(=O)Nc1ccnc(C(F)(F)F)c1] |
| I-28 | 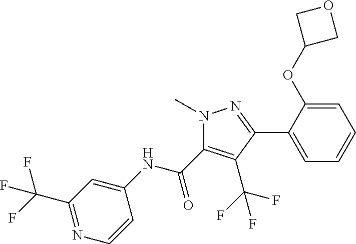 [SMILES: Cn1nc(-c2ccccc2OC2COC2)c(C(F)(F)F)c1C(=O)Nc1ccnc(C(F)(F)F)c1] |
| I-29 | 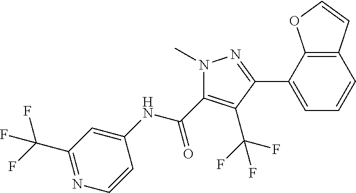 [SMILES: Cn1nc(-c2cccc3ccoc23)c(C(F)(F)F)c1C(=O)Nc1ccnc(C(F)(F)F)c1] |
| I-30 | 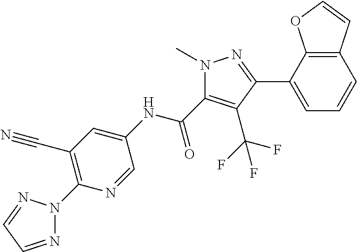 [SMILES: Cn1nc(-c2cccc3ccoc23)c(C(F)(F)F)c1C(=O)Nc1cnc(-n2nccn2)c(C#N)c1] |
| I-31 | 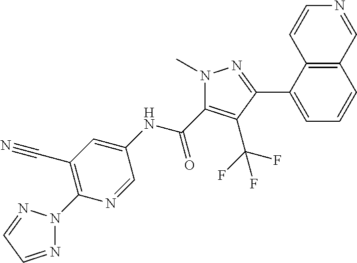 [SMILES: Cn1nc(-c2cccc3cnccc23)c(C(F)(F)F)c1C(=O)Nc1cnc(-n2nccn2)c(C#N)c1] |
| I-32 | 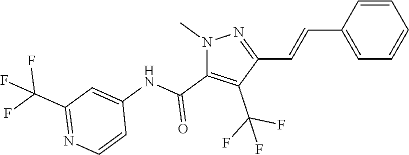 [SMILES: Cn1nc(/C=C/c2ccccc2)c(C(F)(F)F)c1C(=O)Nc1ccnc(C(F)(F)F)c1] |
| I-33 | 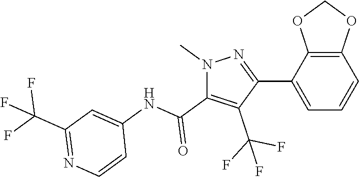 [SMILES: Cn1nc(-c2cccc3c2OCO3)c(C(F)(F)F)c1C(=O)Nc1ccnc(C(F)(F)F)c1] |
| I-34 | 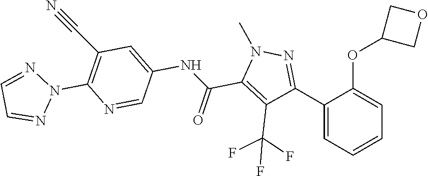 [SMILES: Cn1nc(-c2ccccc2OC2COC2)c(C(F)(F)F)c1C(=O)Nc1cnc(-n2nccn2)c(C#N)c1] |
| I-35 | 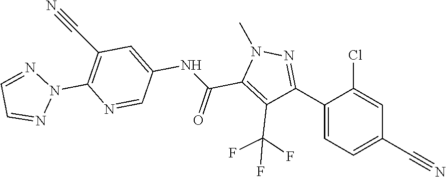 [SMILES: Cn1nc(-c2ccc(C#N)cc2Cl)c(C(F)(F)F)c1C(=O)Nc1cnc(-n2nccn2)c(C#N)c1] |
| I-36 | 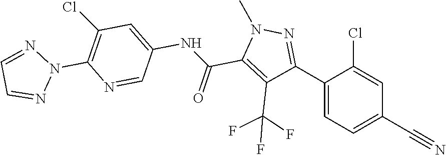 [SMILES: Cn1nc(-c2ccc(C#N)cc2Cl)c(C(F)(F)F)c1C(=O)Nc1cnc(-n2nccn2)c(Cl)c1] |
| I-37 | 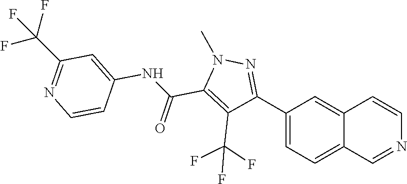 [SMILES: Cn1nc(-c2ccc3cnccc3c2)c(C(F)(F)F)c1C(=O)Nc1ccnc(C(F)(F)F)c1] |
| I-38 | 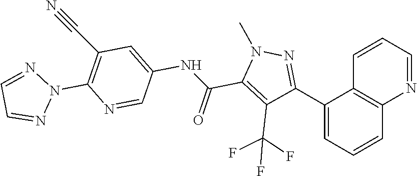 [SMILES: Cn1nc(-c2cccc3ncccc23)c(C(F)(F)F)c1C(=O)Nc1cnc(-n2nccn2)c(C#N)c1] |
| I-39 | 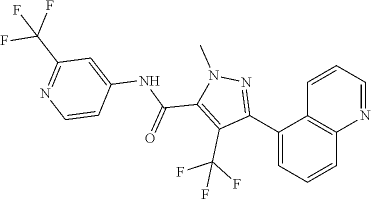 [SMILES: Cn1nc(-c2cccc3ncccc23)c(C(F)(F)F)c1C(=O)Nc1ccnc(C(F)(F)F)c1] |
| I-40 | 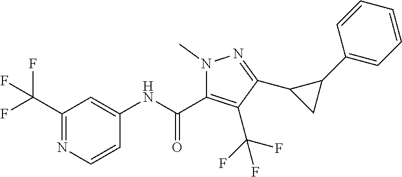 [SMILES: Cn1nc(C2CC2c2ccccc2)c(C(F)(F)F)c1C(=O)Nc1ccnc(C(F)(F)F)c1] |
| I-41 | 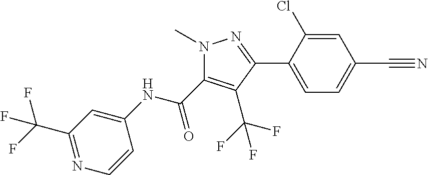 [SMILES: Cn1nc(-c2ccc(C#N)cc2Cl)c(C(F)(F)F)c1C(=O)Nc1ccnc(C(F)(F)F)c1] |
| I-42 | 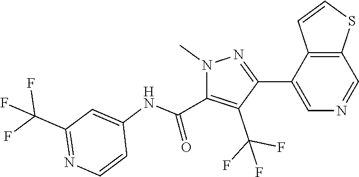 [SMILES: Cn1nc(-c2cncc3sccc23)c(C(F)(F)F)c1C(=O)Nc1ccnc(C(F)(F)F)c1] |
| I-43 | 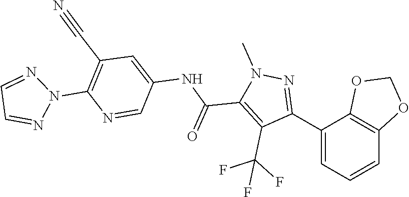 [SMILES: Cn1nc(-c2cccc3c2OCO3)c(C(F)(F)F)c1C(=O)Nc1cnc(-n2nccn2)c(C#N)c1] |
| I-44 | 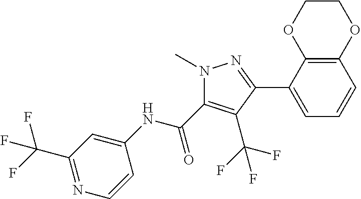 [SMILES: Cn1nc(-c2cccc3c2OCCO3)c(C(F)(F)F)c1C(=O)Nc1ccnc(C(F)(F)F)c1] |
| I-45 | 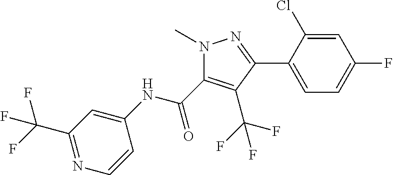 [SMILES: Cn1nc(-c2ccc(F)cc2Cl)c(C(F)(F)F)c1C(=O)Nc1ccnc(C(F)(F)F)c1] |
| I-46 | 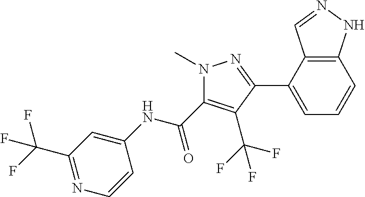 [SMILES: Cn1nc(-c2cccc3[nH]ncc23)c(C(F)(F)F)c1C(=O)Nc1ccnc(C(F)(F)F)c1] |
| I-47 | 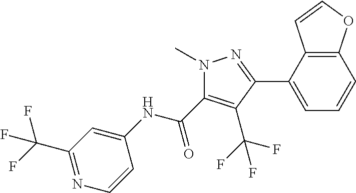 [SMILES: Cn1nc(-c2cccc3occc23)c(C(F)(F)F)c1C(=O)Nc1ccnc(C(F)(F)F)c1] |
| I-48 | 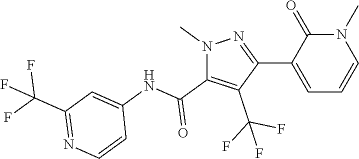 [SMILES: Cn1nc(-c2cccn(C)c2=O)c(C(F)(F)F)c1C(=O)Nc1ccnc(C(F)(F)F)c1] |
| I-49 | 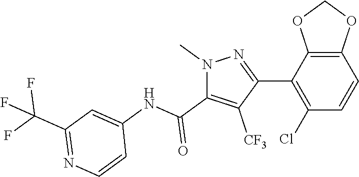 [SMILES: Cn1nc(-c2c(Cl)ccc3c2OCO3)c(C(F)(F)F)c1C(=O)Nc1ccnc(C(F)(F)F)c1] |
| I-50 | 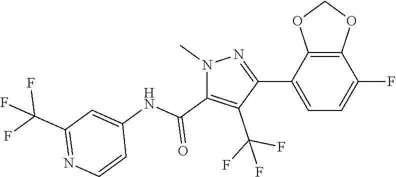 [SMILES: Cn1nc(-c2ccc(F)c3c2OCO3)c(C(F)(F)F)c1C(=O)Nc1ccnc(C(F)(F)F)c1] |
| I-51 | 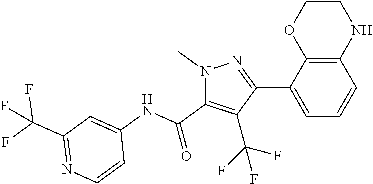 [SMILES: Cn1nc(-c2cccc3c2OCCN3)c(C(F)(F)F)c1C(=O)Nc1ccnc(C(F)(F)F)c1] |
| I-52 | 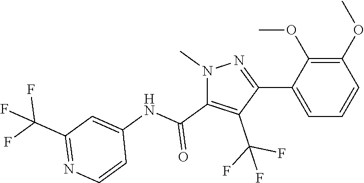 [SMILES: COc1cccc(-c2nn(C)c(C(=O)Nc3ccnc(C(F)(F)F)c3)c2C(F)(F)F)c1OC] |
| I-53 | 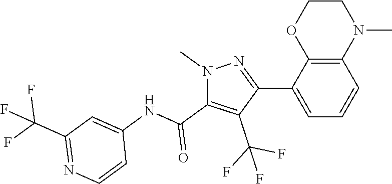 [SMILES: CN1CCOc2c(-c3nn(C)c(C(=O)Nc4ccnc(C(F)(F)F)c4)c3C(F)(F)F)cccc21] |
| I-54 | 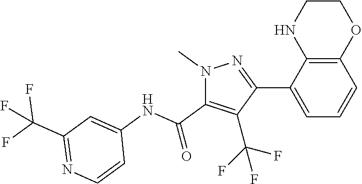 [SMILES: Cn1nc(-c2cccc3c2NCCO3)c(C(F)(F)F)c1C(=O)Nc1ccnc(C(F)(F)F)c1] |
| I-55 | 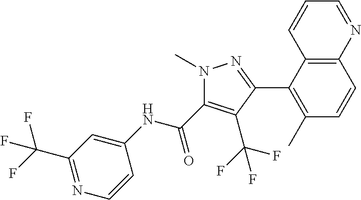 [SMILES: Cc1ccc2ncccc2c1-c1nn(C)c(C(=O)Nc2ccnc(C(F)(F)F)c2)c1C(F)(F)F] |
| I-56 | 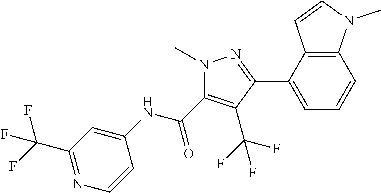 [SMILES: Cn1nc(-c2cccc3c2ccn3C)c(C(F)(F)F)c1C(=O)Nc1ccnc(C(F)(F)F)c1] |
| I-57 | 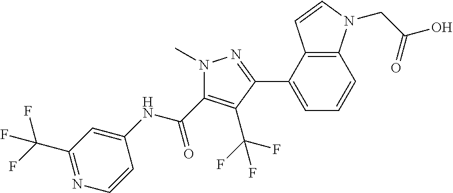 [SMILES: Cn1nc(-c2cccc3c2ccn3CC(=O)O)c(C(F)(F)F)c1C(=O)Nc1ccnc(C(F)(F)F)c1] |
| I-58 | 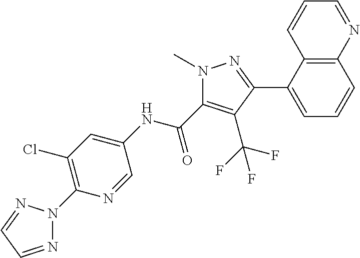 [SMILES: Cn1nc(-c2cccc3ncccc23)c(C(F)(F)F)c1C(=O)Nc1cnc(-n2nccn2)c(Cl)c1] |
| I-59 | 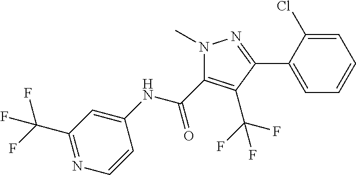 [SMILES: Cn1nc(-c2ccccc2Cl)c(C(F)(F)F)c1C(=O)Nc1ccnc(C(F)(F)F)c1] |
| I-60 | 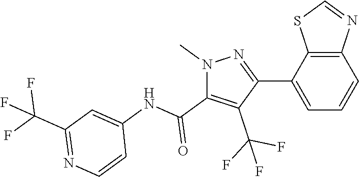 [SMILES: Cn1nc(-c2cccc3ncsc23)c(C(F)(F)F)c1C(=O)Nc1ccnc(C(F)(F)F)c1] |
| I-61 | 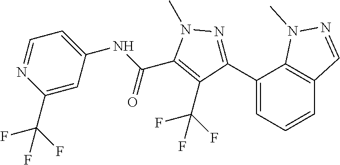 [SMILES: Cn1nc(-c2cccc3cnn(C)c23)c(C(F)(F)F)c1C(=O)Nc1ccnc(C(F)(F)F)c1] |
| I-62 | 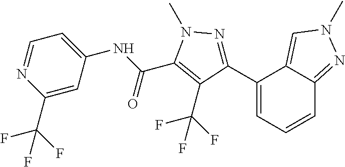 [SMILES: Cn1cc2c(-c3nn(C)c(C(=O)Nc4ccnc(C(F)(F)F)c4)c3C(F)(F)F)cccc2n1] |
| I-63 | 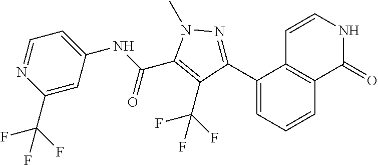 [SMILES: Cn1nc(-c2cccc3c(=O)[nH]ccc23)c(C(F)(F)F)c1C(=O)Nc1ccnc(C(F)(F)F)c1] |
| I-64 | 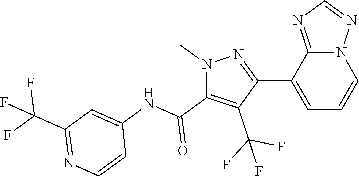 [SMILES: Cn1nc(-c2cccn3ncnc23)c(C(F)(F)F)c1C(=O)Nc1ccnc(C(F)(F)F)c1] |
| I-65 | 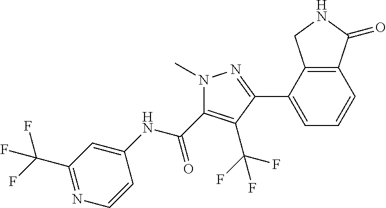 [SMILES: Cn1nc(-c2cccc3c2CNC3=O)c(C(F)(F)F)c1C(=O)Nc1ccnc(C(F)(F)F)c1] |
| I-66 | 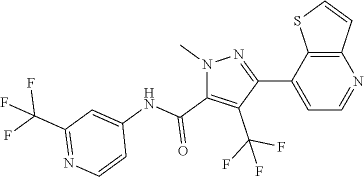 [SMILES: Cn1nc(-c2ccnc3ccsc23)c(C(F)(F)F)c1C(=O)Nc1ccnc(C(F)(F)F)c1] |
| I-67 | 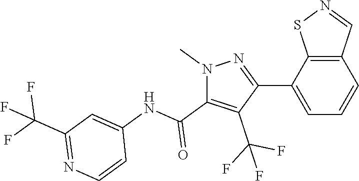 [SMILES: Cn1nc(-c2cccc3cnsc23)c(C(F)(F)F)c1C(=O)Nc1ccnc(C(F)(F)F)c1] |
| I-68 | 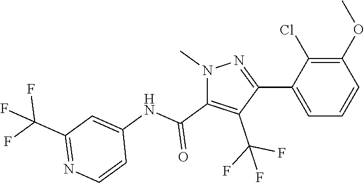 [SMILES: COc1cccc(-c2nn(C)c(C(=O)Nc3ccnc(C(F)(F)F)c3)c2C(F)(F)F)c1Cl] |
| I-69 | 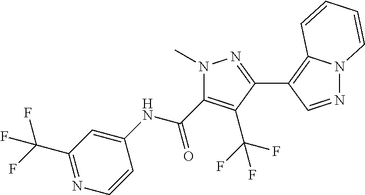 [SMILES: Cn1nc(-c2cnn3ccccc23)c(C(F)(F)F)c1C(=O)Nc1ccnc(C(F)(F)F)c1] |
| I-70 | 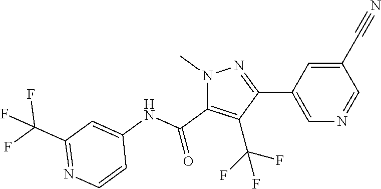 [SMILES: Cn1nc(-c2cncc(C#N)c2)c(C(F)(F)F)c1C(=O)Nc1ccnc(C(F)(F)F)c1] |
| I-71 | 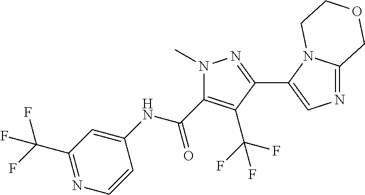 [SMILES: Cn1nc(-c2cnc3n2CCOC3)c(C(F)(F)F)c1C(=O)Nc1ccnc(C(F)(F)F)c1] |
| I-72 | 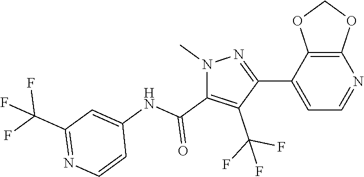 [SMILES: Cn1nc(-c2ccnc3c2OCO3)c(C(F)(F)F)c1C(=O)Nc1ccnc(C(F)(F)F)c1] |
| I-73 | 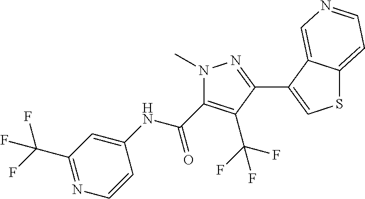 [SMILES: Cn1nc(-c2csc3ccncc23)c(C(F)(F)F)c1C(=O)Nc1ccnc(C(F)(F)F)c1] |
| I-74 | 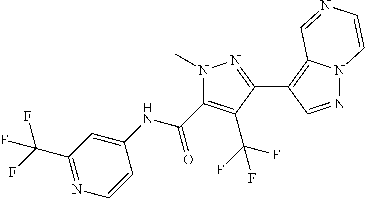 [SMILES: Cn1nc(-c2cnn3ccncc23)c(C(F)(F)F)c1C(=O)Nc1ccnc(C(F)(F)F)c1] |
| I-75 | 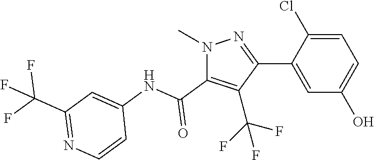 [SMILES: Cn1nc(-c2cc(O)ccc2Cl)c(C(F)(F)F)c1C(=O)Nc1ccnc(C(F)(F)F)c1] |
| I-76 | 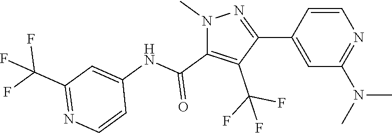 [SMILES: CN(C)c1cc(-c2nn(C)c(C(=O)Nc3ccnc(C(F)(F)F)c3)c2C(F)(F)F)ccn1] |
| I-77 | 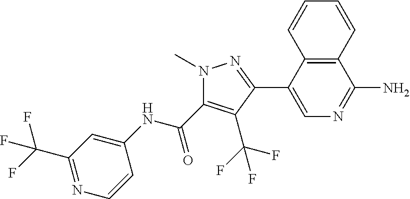 [SMILES: Cn1nc(-c2cnc(N)c3ccccc23)c(C(F)(F)F)c1C(=O)Nc1ccnc(C(F)(F)F)c1] |
| I-78 | 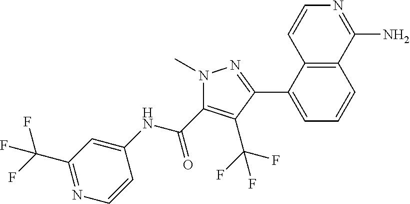 [SMILES: Cn1nc(-c2cccc3c(N)nccc23)c(C(F)(F)F)c1C(=O)Nc1ccnc(C(F)(F)F)c1] |
| I-79 | 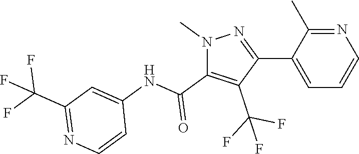 [SMILES: Cc1ncccc1-c1nn(C)c(C(=O)Nc2ccnc(C(F)(F)F)c2)c1C(F)(F)F] |
| I-80 | 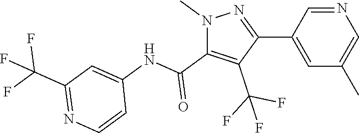 [SMILES: Cc1cncc(-c2nn(C)c(C(=O)Nc3ccnc(C(F)(F)F)c3)c2C(F)(F)F)c1] |
| I-81 | 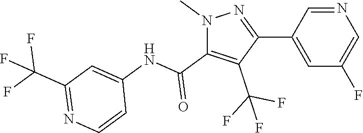 [SMILES: Cn1nc(-c2cncc(F)c2)c(C(F)(F)F)c1C(=O)Nc1ccnc(C(F)(F)F)c1] |
| I-82 | 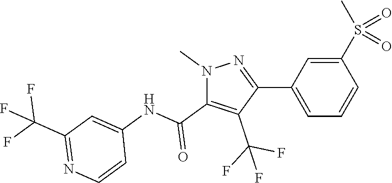 [SMILES: Cn1nc(-c2cccc(S(C)(=O)=O)c2)c(C(F)(F)F)c1C(=O)Nc1ccnc(C(F)(F)F)c1] |
| I-83 | 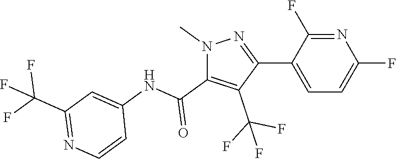 [SMILES: Cn1nc(-c2ccc(F)nc2F)c(C(F)(F)F)c1C(=O)Nc1ccnc(C(F)(F)F)c1] |
| I-84 | 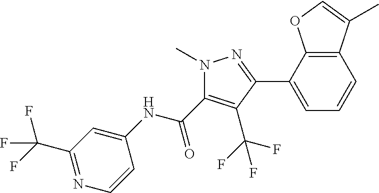 [SMILES: Cc1coc2c(-c3nn(C)c(C(=O)Nc4ccnc(C(F)(F)F)c4)c3C(F)(F)F)cccc12] |
| I-85 | 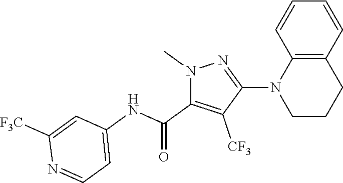 [SMILES: Cn1nc(N2CCCc3ccccc32)c(C(F)(F)F)c1C(=O)Nc1ccnc(C(F)(F)F)c1] |
| I-86 | 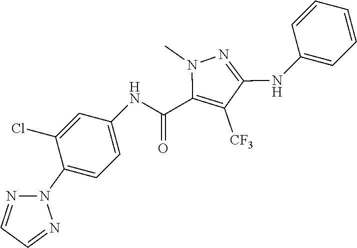 [SMILES: Cn1nc(Nc2ccccc2)c(C(F)(F)F)c1C(=O)Nc1ccc(-n2nccn2)c(Cl)c1] |
| I-87 | 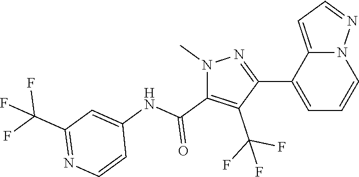 [SMILES: Cn1nc(-c2cccn3nccc23)c(C(F)(F)F)c1C(=O)Nc1ccnc(C(F)(F)F)c1] |
| I-88 | 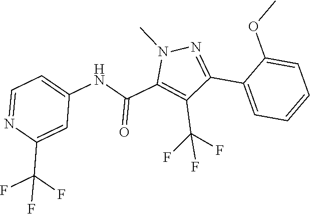 [SMILES: COc1ccccc1-c1nn(C)c(C(=O)Nc2ccnc(C(F)(F)F)c2)c1C(F)(F)F] |
| I-89 | 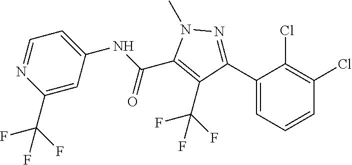 [SMILES: Cn1nc(-c2cccc(Cl)c2Cl)c(C(F)(F)F)c1C(=O)Nc1ccnc(C(F)(F)F)c1] |
| I-90 | 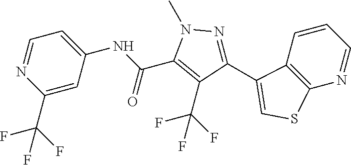 [SMILES: Cn1nc(-c2csc3ncccc23)c(C(F)(F)F)c1C(=O)Nc1ccnc(C(F)(F)F)c1] |
| I-91 | 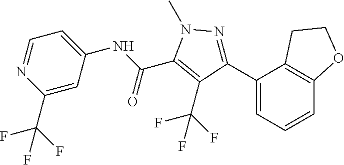 [SMILES: Cn1nc(-c2cccc3c2CCO3)c(C(F)(F)F)c1C(=O)Nc1ccnc(C(F)(F)F)c1] |
| I-92 | 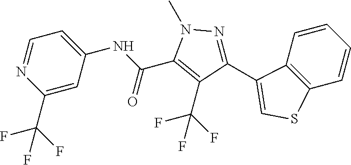 [SMILES: Cn1nc(-c2csc3ccccc23)c(C(F)(F)F)c1C(=O)Nc1ccnc(C(F)(F)F)c1] |
| I-93 | 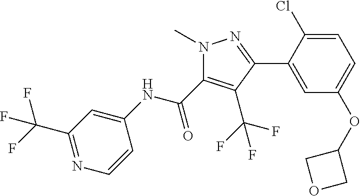 [SMILES: Cn1nc(-c2cc(OC3COC3)ccc2Cl)c(C(F)(F)F)c1C(=O)Nc1ccnc(C(F)(F)F)c1] |
| I-94 | 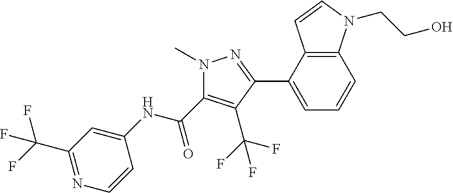 [SMILES: Cn1nc(-c2cccc3c2ccn3CCO)c(C(F)(F)F)c1C(=O)Nc1ccnc(C(F)(F)F)c1] |
| I-95 | 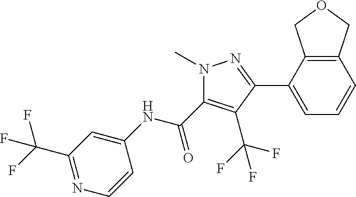 [SMILES: Cn1nc(-c2cccc3c2COC3)c(C(F)(F)F)c1C(=O)Nc1ccnc(C(F)(F)F)c1] |
| I-96 | 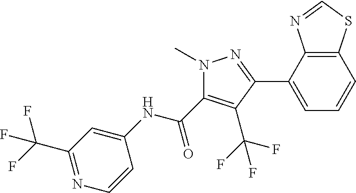 [SMILES: Cn1nc(-c2cccc3scnc23)c(C(F)(F)F)c1C(=O)Nc1ccnc(C(F)(F)F)c1] |
| I-97 | 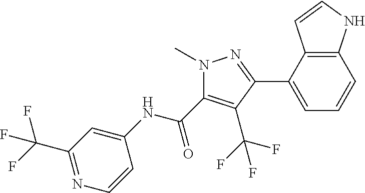 [SMILES: Cn1nc(-c2cccc3[nH]ccc23)c(C(F)(F)F)c1C(=O)Nc1ccnc(C(F)(F)F)c1] |
| I-98 | 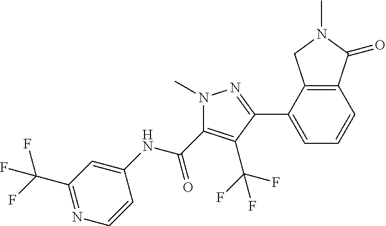 [SMILES: CN1Cc2c(cccc2-c2nn(C)c(C(=O)Nc3ccnc(C(F)(F)F)c3)c2C(F)(F)F)C1=O] |
| I-99 | 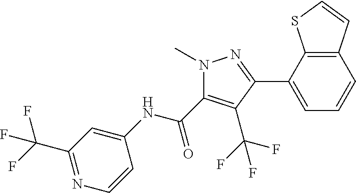 [SMILES: Cn1nc(-c2cccc3ccsc23)c(C(F)(F)F)c1C(=O)Nc1ccnc(C(F)(F)F)c1] |
| I-100 | 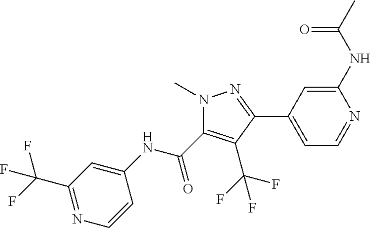 [SMILES: CC(=O)Nc1cc(-c2nn(C)c(C(=O)Nc3ccnc(C(F)(F)F)c3)c2C(F)(F)F)ccn1] |
| I-101 | 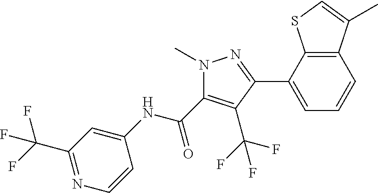 [SMILES: Cc1csc2c(-c3nn(C)c(C(=O)Nc4ccnc(C(F)(F)F)c4)c3C(F)(F)F)cccc12] |
| I-102 | 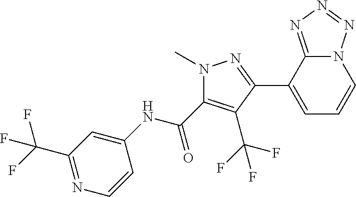 [SMILES: Cn1nc(-c2cccn3nnnc23)c(C(F)(F)F)c1C(=O)Nc1ccnc(C(F)(F)F)c1] |
| I-103 | 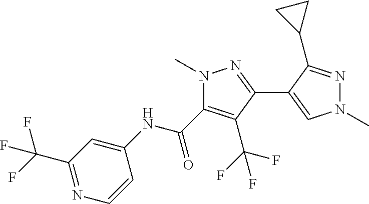 [SMILES: Cn1cc(-c2nn(C)c(C(=O)Nc3ccnc(C(F)(F)F)c3)c2C(F)(F)F)c(C2CC2)n1] |
| I-104 | 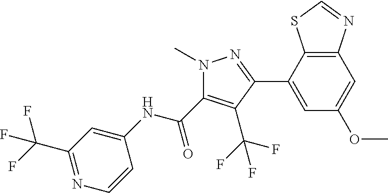 [SMILES: COc1cc(-c2nn(C)c(C(=O)Nc3ccnc(C(F)(F)F)c3)c2C(F)(F)F)c2scnc2c1] |
| I-105 | 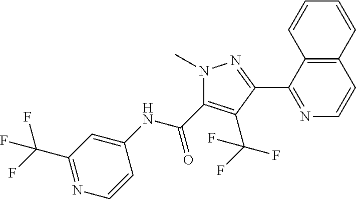 [SMILES: Cn1nc(-c2nccc3ccccc23)c(C(F)(F)F)c1C(=O)Nc1ccnc(C(F)(F)F)c1] |
| I-106 | 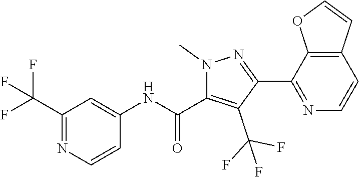 [SMILES: Cn1nc(-c2nccc3ccoc23)c(C(F)(F)F)c1C(=O)Nc1ccnc(C(F)(F)F)c1] |
| I-107 | 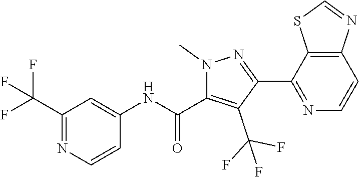 [SMILES: Cn1nc(-c2nccc3ncsc23)c(C(F)(F)F)c1C(=O)Nc1ccnc(C(F)(F)F)c1] |
| I-108 | 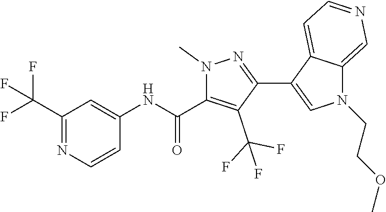 [SMILES: COCCn1cc(-c2nn(C)c(C(=O)Nc3ccnc(C(F)(F)F)c3)c2C(F)(F)F)c2ccncc21] |
| I-109 | 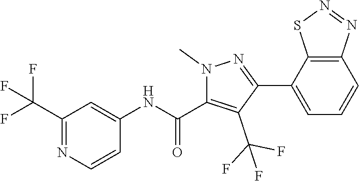 [SMILES: Cn1nc(-c2cccc3nnsc23)c(C(F)(F)F)c1C(=O)Nc1ccnc(C(F)(F)F)c1] |
| I-110 | 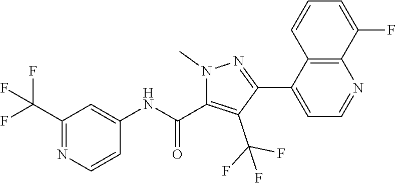 [SMILES: Cn1nc(-c2ccnc3c(F)cccc23)c(C(F)(F)F)c1C(=O)Nc1ccnc(C(F)(F)F)c1] |
| I-111 | 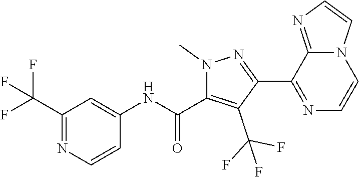 [SMILES: Cn1nc(-c2nccn3ccnc23)c(C(F)(F)F)c1C(=O)Nc1ccnc(C(F)(F)F)c1] |
| I-112 | 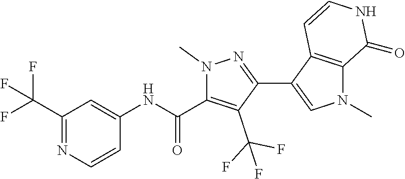 [SMILES: Cn1nc(-c2cn(C)c3c(=O)[nH]ccc23)c(C(F)(F)F)c1C(=O)Nc1ccnc(C(F)(F)F)c1] |
| I-113 | 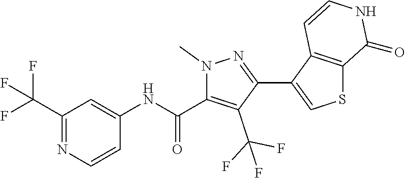 [SMILES: Cn1nc(-c2csc3c(=O)[nH]ccc23)c(C(F)(F)F)c1C(=O)Nc1ccnc(C(F)(F)F)c1] |
| I-114 | 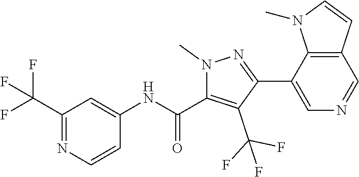 [SMILES: Cn1nc(-c2cncc3ccn(C)c23)c(C(F)(F)F)c1C(=O)Nc1ccnc(C(F)(F)F)c1] |
| I-115 | 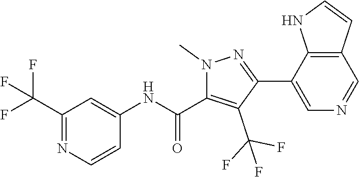 [SMILES: Cn1nc(-c2cncc3cc[nH]c23)c(C(F)(F)F)c1C(=O)Nc1ccnc(C(F)(F)F)c1] |
| I-116 | 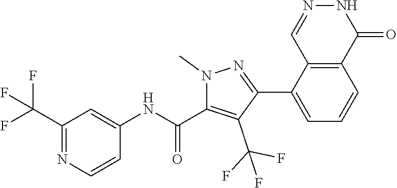 [SMILES: Cn1nc(-c2cccc3c(=O)[nH]ncc23)c(C(F)(F)F)c1C(=O)Nc1ccnc(C(F)(F)F)c1] |
| I-117 | 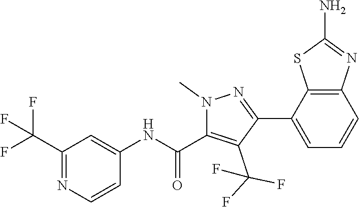 [SMILES: Cn1nc(-c2cccc3nc(N)sc23)c(C(F)(F)F)c1C(=O)Nc1ccnc(C(F)(F)F)c1] |
| I-118 | 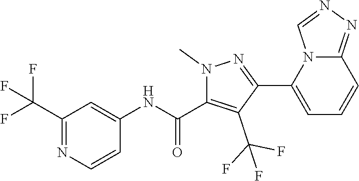 [SMILES: Cn1nc(-c2cccc3nncn23)c(C(F)(F)F)c1C(=O)Nc1ccnc(C(F)(F)F)c1] |
| I-119 | 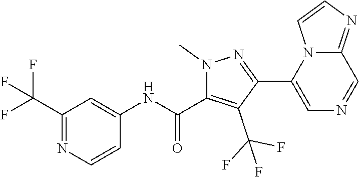 [SMILES: Cn1nc(-c2cncc3nccn23)c(C(F)(F)F)c1C(=O)Nc1ccnc(C(F)(F)F)c1] |
| I-120 | 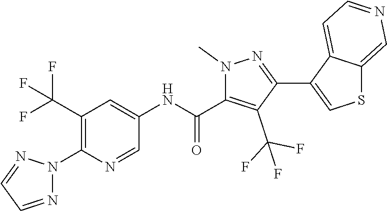 [SMILES: Cn1nc(-c2csc3cnccc23)c(C(F)(F)F)c1C(=O)Nc1cnc(-n2nccn2)c(C(F)(F)F)c1] |
| I-121 | 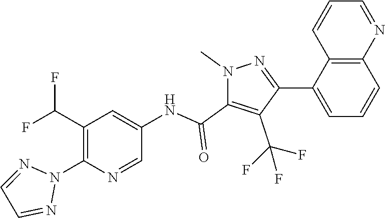 [SMILES: Cn1nc(-c2cccc3ncccc23)c(C(F)(F)F)c1C(=O)Nc1cnc(-n2nccn2)c(C(F)F)c1] |
| I-122 | 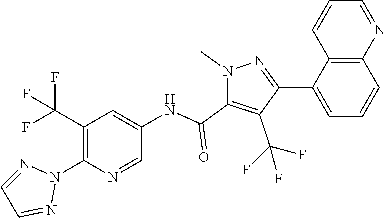 [SMILES: Cn1nc(-c2cccc3ncccc23)c(C(F)(F)F)c1C(=O)Nc1cnc(-n2nccn2)c(C(F)(F)F)c1] |
| I-123 | 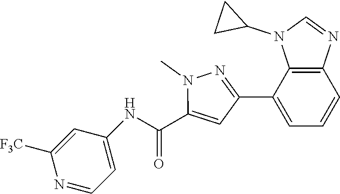 [SMILES: Cn1nc(-c2cccc3ncn(C4CC4)c23)cc1C(=O)Nc1ccnc(C(F)(F)F)c1] |
| I-124 | 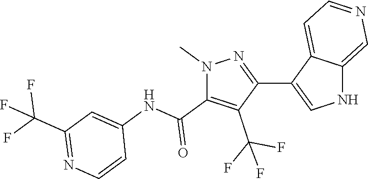 [SMILES: Cn1nc(-c2c[nH]c3cnccc23)c(C(F)(F)F)c1C(=O)Nc1ccnc(C(F)(F)F)c1] |
| I-125 | 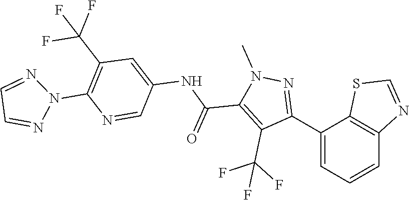 [SMILES: Cn1nc(-c2cccc3ncsc23)c(C(F)(F)F)c1C(=O)Nc1cnc(-n2nccn2)c(C(F)(F)F)c1] |
| I-126 | 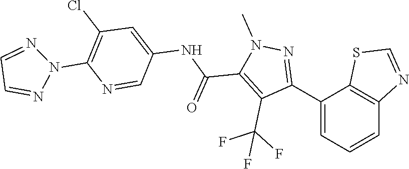 [SMILES: Cn1nc(-c2cccc3ncsc23)c(C(F)(F)F)c1C(=O)Nc1cnc(-n2nccn2)c(Cl)c1] |
| I-127 | 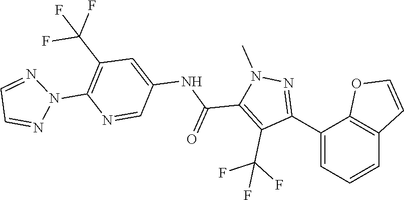 [SMILES: Cn1nc(-c2cccc3ccoc23)c(C(F)(F)F)c1C(=O)Nc1cnc(-n2nccn2)c(C(F)(F)F)c1] |
| I-128 | 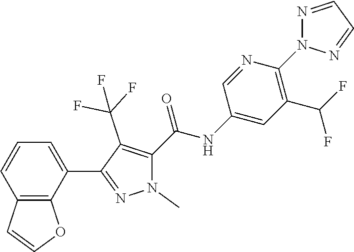 [SMILES: Cn1nc(-c2cccc3ccoc23)c(C(F)(F)F)c1C(=O)Nc1cnc(-n2nccn2)c(C(F)F)c1] |
| I-129 | 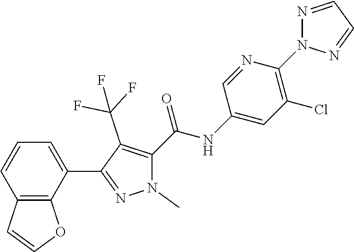 [SMILES: Cn1nc(-c2cccc3ccoc23)c(C(F)(F)F)c1C(=O)Nc1cnc(-n2nccn2)c(Cl)c1] |
| I-130 | 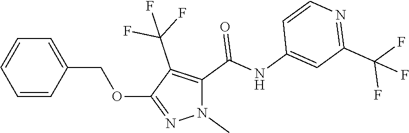 [SMILES: Cn1nc(OCc2ccccc2)c(C(F)(F)F)c1C(=O)Nc1ccnc(C(F)(F)F)c1] |
| I-131 | 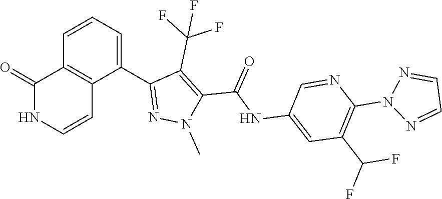 [SMILES: Cn1nc(-c2cccc3c(=O)[nH]ccc23)c(C(F)(F)F)c1C(=O)Nc1cnc(-n2nccn2)c(C(F)F)c1] |
| I-132 | 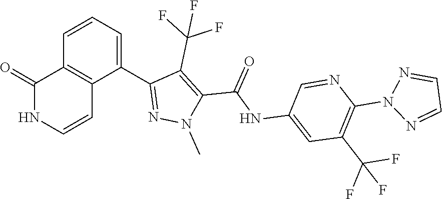 [SMILES: Cn1nc(-c2cccc3c(=O)[nH]ccc23)c(C(F)(F)F)c1C(=O)Nc1cnc(-n2nccn2)c(C(F)(F)F)c1] |
| I-133 | 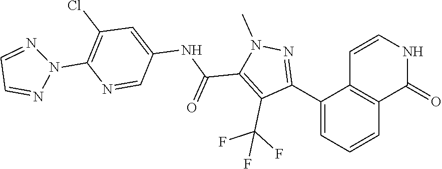 [SMILES: Cn1nc(-c2cccc3c(=O)[nH]ccc23)c(C(F)(F)F)c1C(=O)Nc1cnc(-n2nccn2)c(Cl)c1] |
| I-134 | 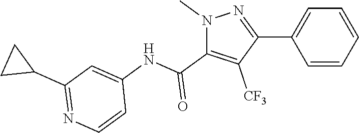 [SMILES: Cn1nc(-c2ccccc2)c(C(F)(F)F)c1C(=O)Nc1ccnc(C2CC2)c1] |
| I-135 | 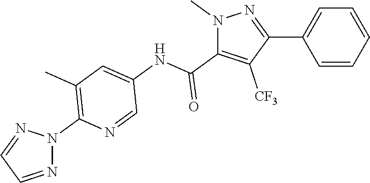 [SMILES: Cc1cc(NC(=O)c2c(C(F)(F)F)c(-c3ccccc3)nn2C)cnc1-n1nccn1] |
| I-136 | 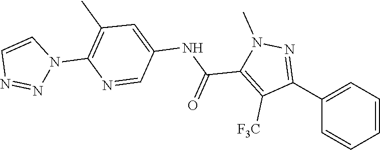 [SMILES: Cc1cc(NC(=O)c2c(C(F)(F)F)c(-c3ccccc3)nn2C)cnc1-n1ccnn1] |
| I-137 | 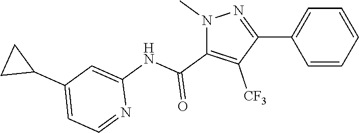 [SMILES: Cn1nc(-c2ccccc2)c(C(F)(F)F)c1C(=O)Nc1cc(C2CC2)ccn1] |
| I-138 | 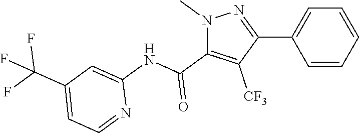 [SMILES: Cn1nc(-c2ccccc2)c(C(F)(F)F)c1C(=O)Nc1cc(C(F)(F)F)ccn1] |
| I-139 | 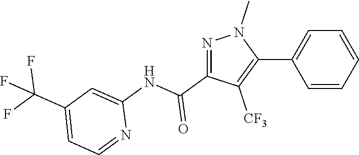 [SMILES: Cn1nc(C(=O)Nc2cc(C(F)(F)F)ccn2)c(C(F)(F)F)c1-c1ccccc1] |
| I-140 | 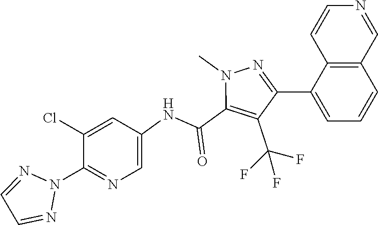 [SMILES: Cn1nc(-c2cccc3cnccc23)c(C(F)(F)F)c1C(=O)Nc1cnc(-n2nccn2)c(Cl)c1] |
| I-141 | 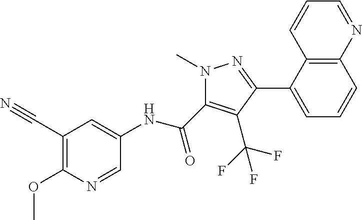 [SMILES: COc1ncc(NC(=O)c2c(C(F)(F)F)c(-c3cccc4ncccc34)nn2C)cc1C#N] |
| I-142 | 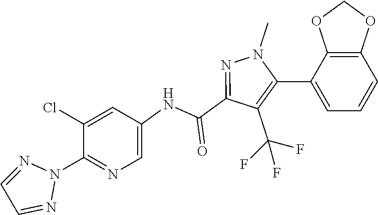 [SMILES: Cn1nc(C(=O)Nc2cnc(-n3nccn3)c(Cl)c2)c(C(F)(F)F)c1-c1cccc2c1OCO2] |
| I-143 | 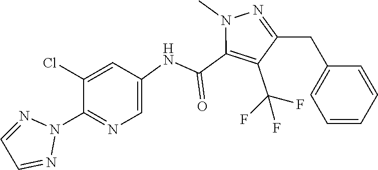 [SMILES: Cn1nc(Cc2ccccc2)c(C(F)(F)F)c1C(=O)Nc1cnc(-n2nccn2)c(Cl)c1] |
| I-144 | 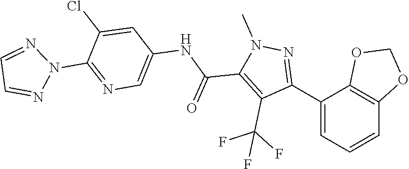 [SMILES: Cn1nc(-c2cccc3c2OCO3)c(C(F)(F)F)c1C(=O)Nc1cnc(-n2nccn2)c(Cl)c1] |
| I-145 | 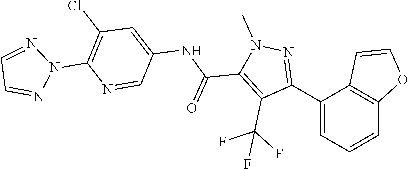 [SMILES: Cn1nc(-c2cccc3occc23)c(C(F)(F)F)c1C(=O)Nc1cnc(-n2nccn2)c(Cl)c1] |
| I-146 | 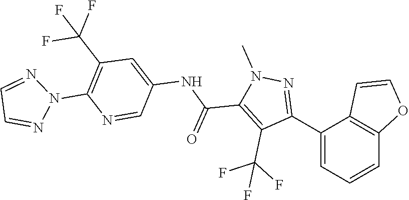 [SMILES: Cn1nc(-c2cccc3occc23)c(C(F)(F)F)c1C(=O)Nc1cnc(-n2nccn2)c(C(F)(F)F)c1] |
| I-147 | 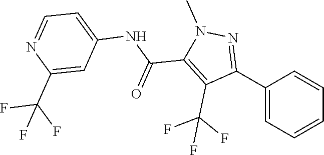 [SMILES: Cn1nc(-c2ccccc2)c(C(F)(F)F)c1C(=O)Nc1ccnc(C(F)(F)F)c1] |
| I-148 | 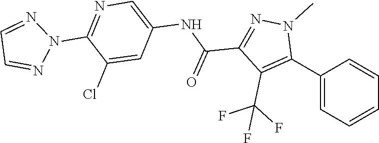 [SMILES: Cn1nc(C(=O)Nc2cnc(-n3nccn3)c(Cl)c2)c(C(F)(F)F)c1-c1ccccc1] |
| I-149 | 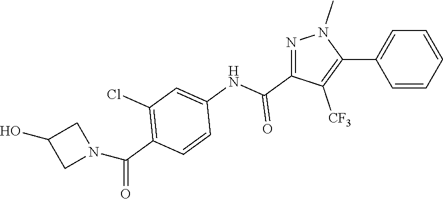 [SMILES: Cn1nc(C(=O)Nc2ccc(C(=O)N3CC(O)C3)c(Cl)c2)c(C(F)(F)F)c1-c1ccccc1] |
| I-150 | 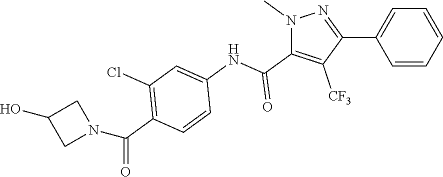 [SMILES: Cn1nc(-c2ccccc2)c(C(F)(F)F)c1C(=O)Nc1ccc(C(=O)N2CC(O)C2)c(Cl)c1] |
| I-151 | 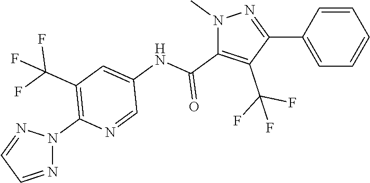 [SMILES: Cn1nc(-c2ccccc2)c(C(F)(F)F)c1C(=O)Nc1cnc(-n2nccn2)c(C(F)(F)F)c1] |
| I-152 | 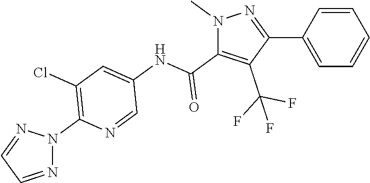 [SMILES: Cn1nc(-c2ccccc2)c(C(F)(F)F)c1C(=O)Nc1cnc(-n2nccn2)c(Cl)c1] |
| I-153 | 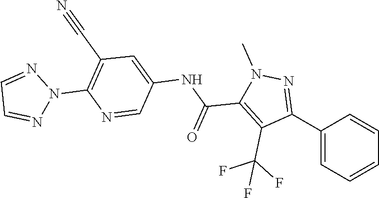 [SMILES: Cn1nc(-c2ccccc2)c(C(F)(F)F)c1C(=O)Nc1cnc(-n2nccn2)c(C#N)c1] |
| I-154 | 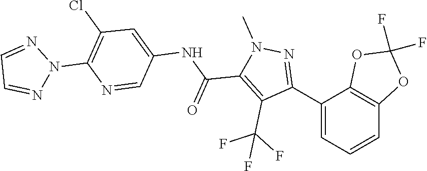 [SMILES: Cn1nc(-c2cccc3c2OC(F)(F)O3)c(C(F)(F)F)c1C(=O)Nc1cnc(-n2nccn2)c(Cl)c1] |
| I-155 | 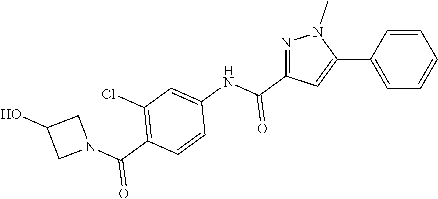 [SMILES: Cn1nc(C(=O)Nc2ccc(C(=O)N3CC(O)C3)c(Cl)c2)cc1-c1ccccc1] |
| I-156 | 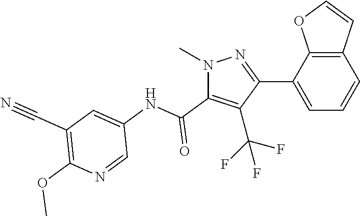 [SMILES: COc1ncc(NC(=O)c2c(C(F)(F)F)c(-c3cccc4ccoc34)nn2C)cc1C#N] |
| I-157 | 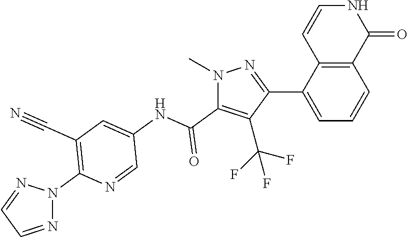 [SMILES: Cn1nc(-c2cccc3c(=O)[nH]ccc23)c(C(F)(F)F)c1C(=O)Nc1cnc(-n2nccn2)c(C#N)c1] |
| I-158 | 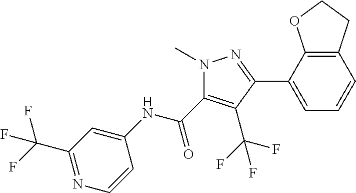 [SMILES: Cn1nc(-c2cccc3c2OCC3)c(C(F)(F)F)c1C(=O)Nc1ccnc(C(F)(F)F)c1] |
| I-159 | 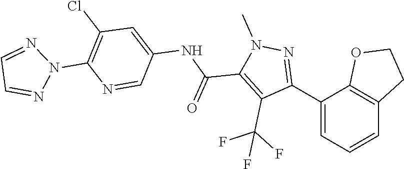 [SMILES: Cn1nc(-c2cccc3c2OCC3)c(C(F)(F)F)c1C(=O)Nc1cnc(-n2nccn2)c(Cl)c1] |
| I-160 | 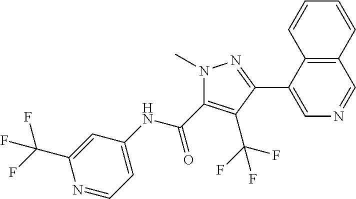 [SMILES: Cn1nc(-c2cncc3ccccc23)c(C(F)(F)F)c1C(=O)Nc1ccnc(C(F)(F)F)c1] |
| I-161 | 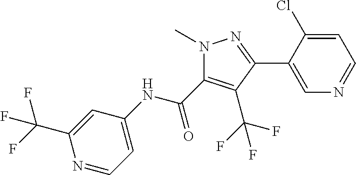 [SMILES: Cn1nc(-c2cnccc2Cl)c(C(F)(F)F)c1C(=O)Nc1ccnc(C(F)(F)F)c1] |
| I-162 | 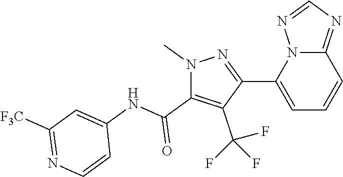 [SMILES: Cn1nc(-c2cccc3ncnn23)c(C(F)(F)F)c1C(=O)Nc1ccnc(C(F)(F)F)c1] |
| I-163 | 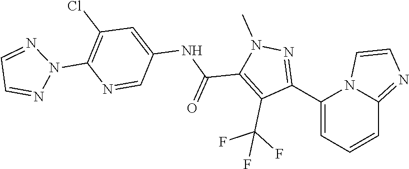 [SMILES: Cn1nc(-c2cccc3nccn23)c(C(F)(F)F)c1C(=O)Nc1cnc(-n2nccn2)c(Cl)c1] |
| I-165 | 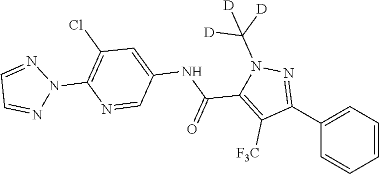 [SMILES: [2H]C([2H])([2H])n1nc(-c2ccccc2)c(C(F)(F)F)c1C(=O)Nc1cnc(-n2nccn2)c(Cl)c1] |
| I-166 | 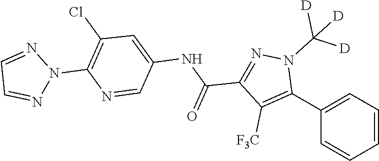 [SMILES: [2H]C([2H])([2H])n1nc(C(=O)Nc2cnc(-n3nccn3)c(Cl)c2)c(C(F)(F)F)c1-c1ccccc1] |
| I-167 | 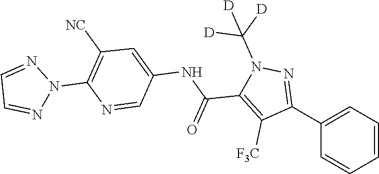 [SMILES: [2H]C([2H])([2H])n1nc(-c2ccccc2)c(C(F)(F)F)c1C(=O)Nc1cnc(-n2nccn2)c(C#N)c1] |
| I-168 | 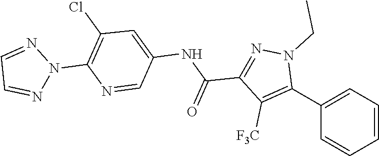 [SMILES: CCn1nc(C(=O)Nc2cnc(-n3nccn3)c(Cl)c2)c(C(F)(F)F)c1-c1ccccc1] |
| I-169 | 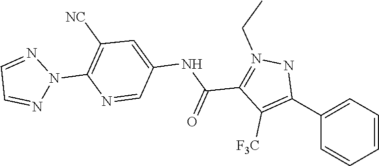 |
| I-170 | 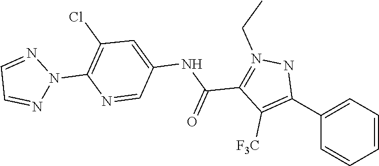 |
| I-171 | 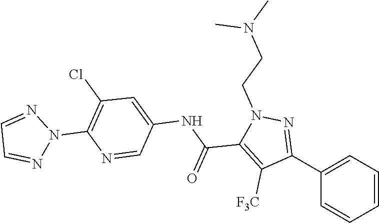 [SMILES: CN(C)CCn1nc(-c2ccccc2)c(C(F)(F)F)c1C(=O)Nc1cnc(-n2nccn2)c(Cl)c1] |
| I-172 | 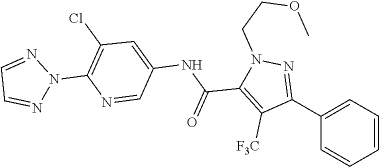 [SMILES: COCCn1nc(-c2ccccc2)c(C(F)(F)F)c1C(=O)Nc1cnc(-n2nccn2)c(Cl)c1] |
| I-173 | 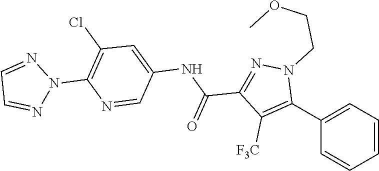 [SMILES: COCCn1nc(C(=O)Nc2cnc(-n3nccn3)c(Cl)c2)c(C(F)(F)F)c1-c1ccccc1] |
| I-174 | 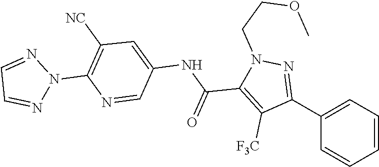 [SMILES: COCCn1nc(-c2ccccc2)c(C(F)(F)F)c1C(=O)Nc1cnc(-n2nccn2)c(C#N)c1] |
| I-175 | 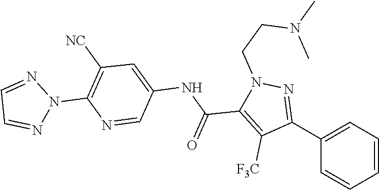 [SMILES: CN(C)CCn1nc(-c2ccccc2)c(C(F)(F)F)c1C(=O)Nc1cnc(-n2nccn2)c(C#N)c1] |
| I-176 | 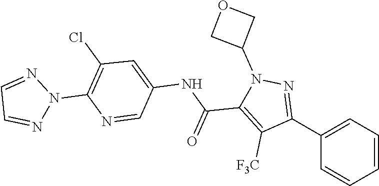 [SMILES: O=C(Nc1cnc(-n2nccn2)c(Cl)c1)c1c(C(F)(F)F)c(-c2ccccc2)nn1C1COC1] |
| I-177 | 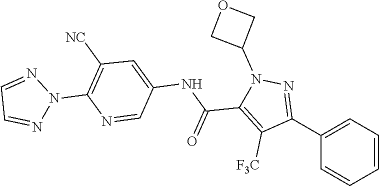 [SMILES: N#Cc1cc(NC(=O)c2c(C(F)(F)F)c(-c3ccccc3)nn2C2COC2)cnc1-n1nccn1] |
| I-178 | 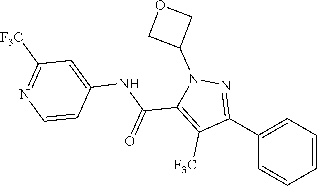 [SMILES: O=C(Nc1ccnc(C(F)(F)F)c1)c1c(C(F)(F)F)c(-c2ccccc2)nn1C1COC1] |
| I-179 | 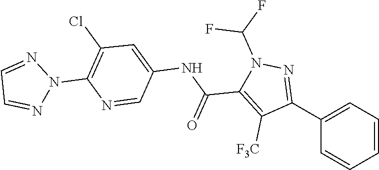 [SMILES: O=C(Nc1cnc(-n2nccn2)c(Cl)c1)c1c(C(F)(F)F)c(-c2ccccc2)nn1C(F)F] |
| I-180 | 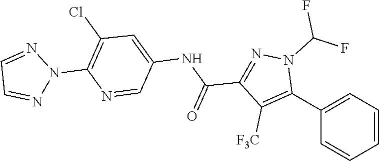 [SMILES: O=C(Nc1cnc(-n2nccn2)c(Cl)c1)c1nn(C(F)F)c(-c2ccccc2)c1C(F)(F)F] |
| I-181 | 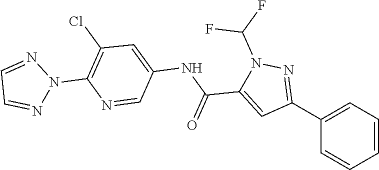 [SMILES: O=C(Nc1cnc(-n2nccn2)c(Cl)c1)c1cc(-c2ccccc2)nn1C(F)F] |
| I-182 | 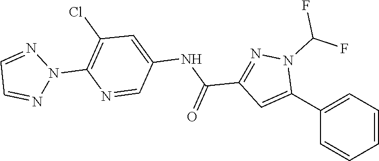 [SMILES: O=C(Nc1cnc(-n2nccn2)c(Cl)c1)c1cc(-c2ccccc2)n(C(F)F)n1] |
| I-183 | 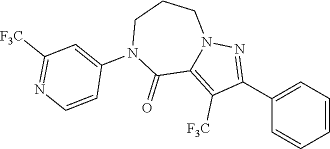 [SMILES: O=C1c2c(C(F)(F)F)c(-c3ccccc3)nn2CCCN1c1ccnc(C(F)(F)F)c1] |
| I-184 | 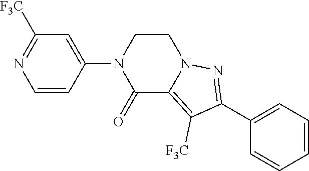 [SMILES: O=C1c2c(C(F)(F)F)c(-c3ccccc3)nn2CCN1c1ccnc(C(F)(F)F)c1] |
| I-185 | 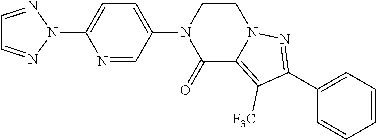 [SMILES: O=C1c2c(C(F)(F)F)c(-c3ccccc3)nn2CCN1c1ccc(-n2nccn2)nc1] |
| I-186 | 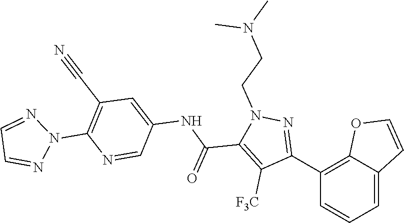 [SMILES: CN(C)CCn1nc(-c2cccc3ccoc23)c(C(F)(F)F)c1C(=O)Nc1cnc(-n2nccn2)c(C#N)c1] |
| I-187 | 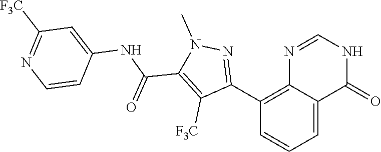 [SMILES: Cn1nc(-c2cccc3c(=O)[nH]cnc23)c(C(F)(F)F)c1C(=O)Nc1ccnc(C(F)(F)F)c1] |
| I-188 | 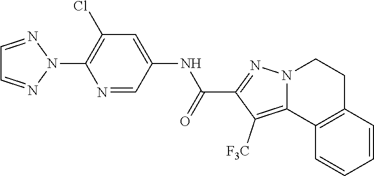 [SMILES: O=C(Nc1cnc(-n2nccn2)c(Cl)c1)c1nn2c(c1C(F)(F)F)-c1ccccc1CC2] |
| I-189 | 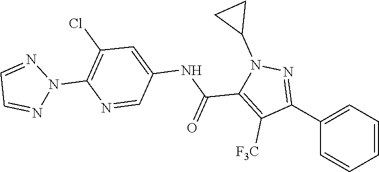 [SMILES: O=C(Nc1cnc(-n2nccn2)c(Cl)c1)c1c(C(F)(F)F)c(-c2ccccc2)nn1C1CC1] |
| I-190 | 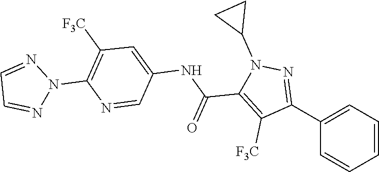 [SMILES: O=C(Nc1cnc(-n2nccn2)c(C(F)(F)F)c1)c1c(C(F)(F)F)c(-c2ccccc2)nn1C1CC1] |
| I-191 | 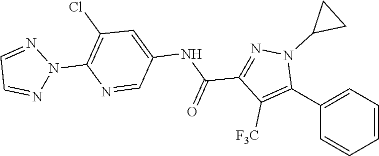 [SMILES: O=C(Nc1cnc(-n2nccn2)c(Cl)c1)c1nn(C2CC2)c(-c2ccccc2)c1C(F)(F)F] |
| I-192 | 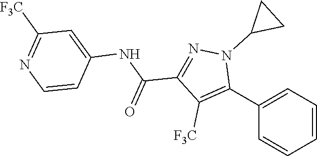 [SMILES: O=C(Nc1ccnc(C(F)(F)F)c1)c1nn(C2CC2)c(-c2ccccc2)c1C(F)(F)F] |
| I-193 | 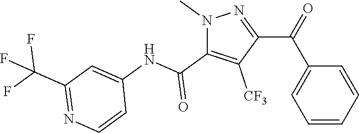 [SMILES: Cn1nc(C(=O)c2ccccc2)c(C(F)(F)F)c1C(=O)Nc1ccnc(C(F)(F)F)c1] |
| I-194 | 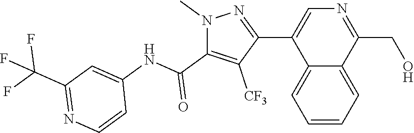 [SMILES: Cn1nc(-c2cnc(CO)c3ccccc23)c(C(F)(F)F)c1C(=O)Nc1ccnc(C(F)(F)F)c1] |
| I-195 | 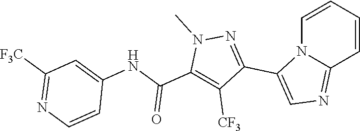 [SMILES: Cn1nc(-c2cnc3ccccn23)c(C(F)(F)F)c1C(=O)Nc1ccnc(C(F)(F)F)c1] |
| I-196 | 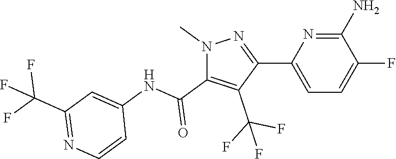 [SMILES: Cn1nc(-c2ccc(F)c(N)n2)c(C(F)(F)F)c1C(=O)Nc1ccnc(C(F)(F)F)c1] |
| I-197 | 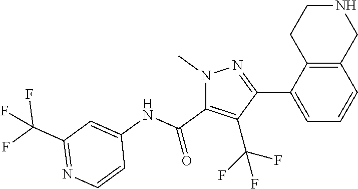 [SMILES: Cn1nc(-c2cccc3c2CCNC3)c(C(F)(F)F)c1C(=O)Nc1ccnc(C(F)(F)F)c1] |
| I-198 | 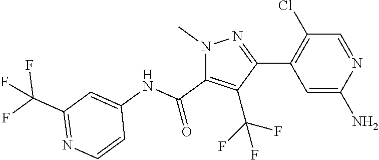 [SMILES: Cn1nc(-c2cc(N)ncc2Cl)c(C(F)(F)F)c1C(=O)Nc1ccnc(C(F)(F)F)c1] |
| I-199 | 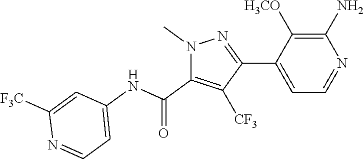 [SMILES: COc1c(-c2nn(C)c(C(=O)Nc3ccnc(C(F)(F)F)c3)c2C(F)(F)F)ccnc1N] |
| I-200 | 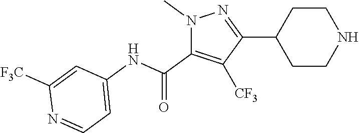 [SMILES: Cn1nc(C2CCNCC2)c(C(F)(F)F)c1C(=O)Nc1ccnc(C(F)(F)F)c1] |
| I-201 | 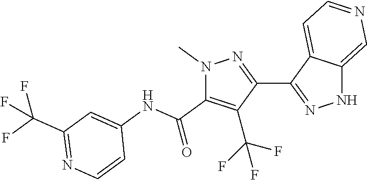 [SMILES: Cn1nc(-c2n[nH]c3cnccc23)c(C(F)(F)F)c1C(=O)Nc1ccnc(C(F)(F)F)c1] |
| I-202 | 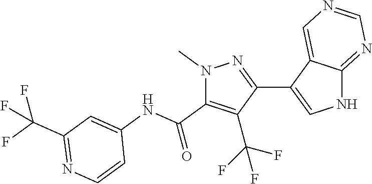 [SMILES: Cn1nc(-c2c[nH]c3ncncc23)c(C(F)(F)F)c1C(=O)Nc1ccnc(C(F)(F)F)c1] |
| I-203 | 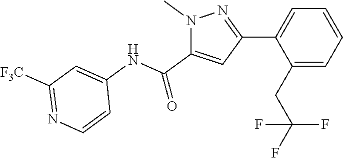 [SMILES: Cn1nc(-c2ccccc2CC(F)(F)F)cc1C(=O)Nc1ccnc(C(F)(F)F)c1] |
| I-204 | 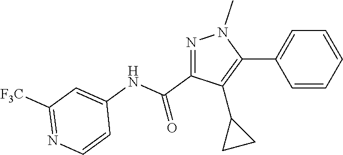 [SMILES: Cn1nc(C(=O)Nc2ccnc(C(F)(F)F)c2)c(C2CC2)c1-c1ccccc1] |
| I-205 | 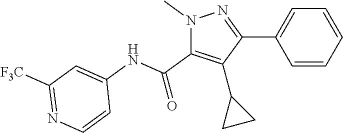 [SMILES: Cn1nc(-c2ccccc2)c(C2CC2)c1C(=O)Nc1ccnc(C(F)(F)F)c1] |
| I-206 | 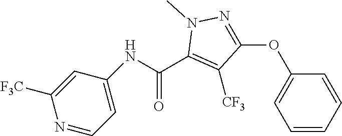 [SMILES: Cn1nc(Oc2ccccc2)c(C(F)(F)F)c1C(=O)Nc1ccnc(C(F)(F)F)c1] |
| I-207 | 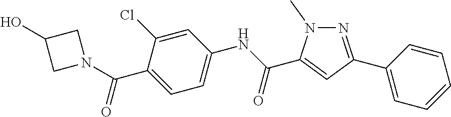 [SMILES: Cn1nc(-c2ccccc2)cc1C(=O)Nc1ccc(C(=O)N2CC(O)C2)c(Cl)c1] |
| I-208 | 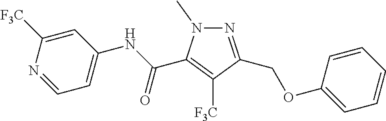 [SMILES: Cn1nc(COc2ccccc2)c(C(F)(F)F)c1C(=O)Nc1ccnc(C(F)(F)F)c1] |
| I-209 | 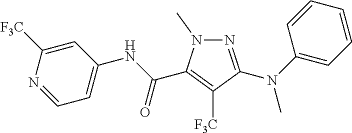 [SMILES: CN(c1ccccc1)c1nn(C)c(C(=O)Nc2ccnc(C(F)(F)F)c2)c1C(F)(F)F] |
| I-210 | 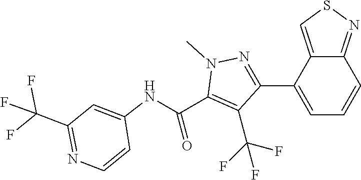 [SMILES: Cn1nc(-c2cccc3nscc23)c(C(F)(F)F)c1C(=O)Nc1ccnc(C(F)(F)F)c1] |
| I-211 | 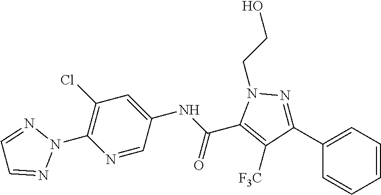 [SMILES: O=C(Nc1cnc(-n2nccn2)c(Cl)c1)c1c(C(F)(F)F)c(-c2ccccc2)nn1CCO] |
| I-222 | 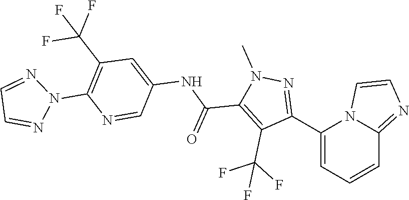 [SMILES: Cn1nc(-c2cccc3nccn23)c(C(F)(F)F)c1C(=O)Nc1cnc(-n2nccn2)c(C(F)(F)F)c1] |
| I-223 | 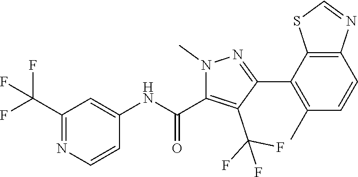 [SMILES: Cc1ccc2ncsc2c1-c1nn(C)c(C(=O)Nc2ccnc(C(F)(F)F)c2)c1C(F)(F)F] |
| I-224 | 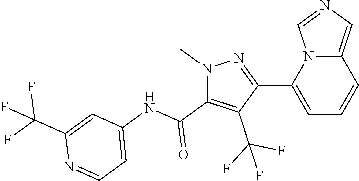 [SMILES: Cn1nc(-c2cccc3cncn23)c(C(F)(F)F)c1C(=O)Nc1ccnc(C(F)(F)F)c1] |
| I-225 | 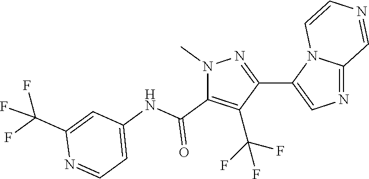 [SMILES: Cn1nc(-c2cnc3cnccn23)c(C(F)(F)F)c1C(=O)Nc1ccnc(C(F)(F)F)c1] |
| I-226 | 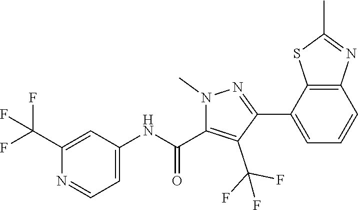 [SMILES: Cc1nc2cccc(-c3nn(C)c(C(=O)Nc4ccnc(C(F)(F)F)c4)c3C(F)(F)F)c2s1] |
| I-227 | 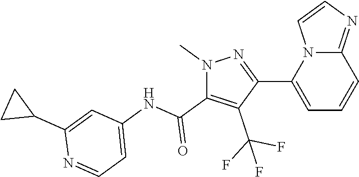 [SMILES: Cn1nc(-c2cccc3nccn23)c(C(F)(F)F)c1C(=O)Nc1ccnc(C2CC2)c1] |
| I-228 | 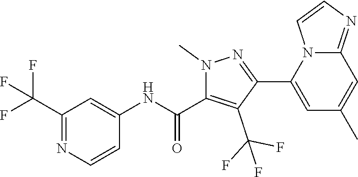 [SMILES: Cc1cc(-c2nn(C)c(C(=O)Nc3ccnc(C(F)(F)F)c3)c2C(F)(F)F)n2ccnc2c1] |
| I-229 | 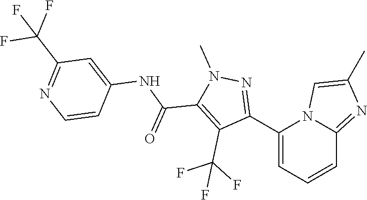 [SMILES: Cc1cn2c(-c3nn(C)c(C(=O)Nc4ccnc(C(F)(F)F)c4)c3C(F)(F)F)cccc2n1] |
| I-230 | 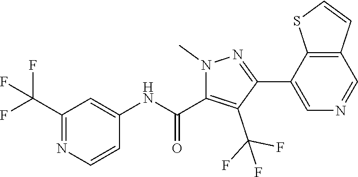 [SMILES: Cn1nc(-c2cncc3ccsc23)c(C(F)(F)F)c1C(=O)Nc1ccnc(C(F)(F)F)c1] |
| I-231 | 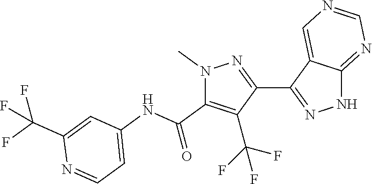 [SMILES: Cn1nc(-c2n[nH]c3ncncc23)c(C(F)(F)F)c1C(=O)Nc1ccnc(C(F)(F)F)c1] |
| I-232 | 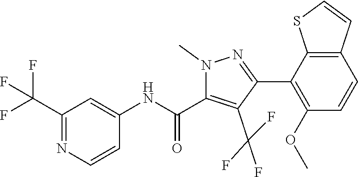 [SMILES: COc1ccc2ccsc2c1-c1nn(C)c(C(=O)Nc2ccnc(C(F)(F)F)c2)c1C(F)(F)F] |
| I-233 | 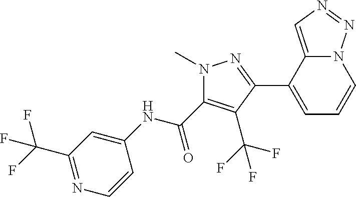 [SMILES: Cn1nc(-c2cccn3nncc23)c(C(F)(F)F)c1C(=O)Nc1ccnc(C(F)(F)F)c1] |
| I-234 | 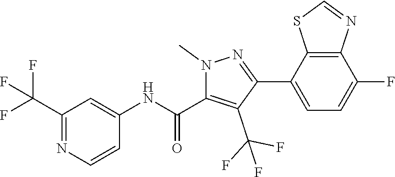 [SMILES: Cn1nc(-c2ccc(F)c3ncsc23)c(C(F)(F)F)c1C(=O)Nc1ccnc(C(F)(F)F)c1] |
| I-235 | 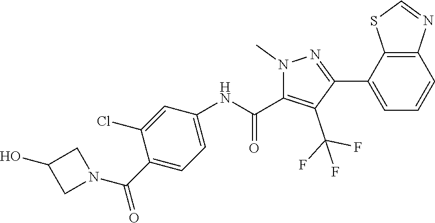 [SMILES: Cn1nc(-c2cccc3ncsc23)c(C(F)(F)F)c1C(=O)Nc1ccc(C(=O)N2CC(O)C2)c(Cl)c1] |
| I-236 | 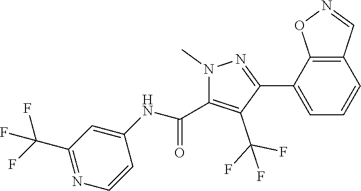 [SMILES: Cn1nc(-c2cccc3cnoc23)c(C(F)(F)F)c1C(=O)Nc1ccnc(C(F)(F)F)c1] |
| I-237 | 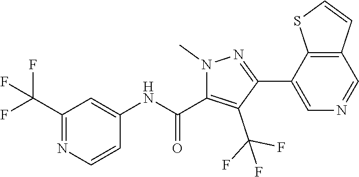 [SMILES: Cn1nc(-c2cncc3ccsc23)c(C(F)(F)F)c1C(=O)Nc1ccnc(C(F)(F)F)c1] |
| I-238 | 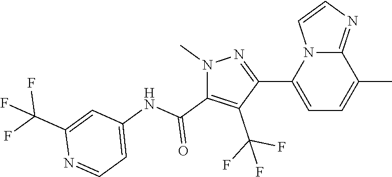 [SMILES: Cc1ccc(-c2nn(C)c(C(=O)Nc3ccnc(C(F)(F)F)c3)c2C(F)(F)F)n2ccnc12] |
| I-239 | 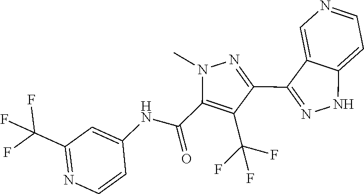 [SMILES: Cn1nc(-c2n[nH]c3ccncc23)c(C(F)(F)F)c1C(=O)Nc1ccnc(C(F)(F)F)c1] |
| I-240 | 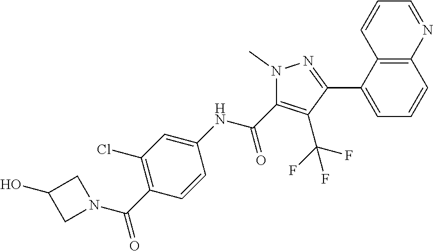 [SMILES: Cn1nc(-c2cccc3ncccc23)c(C(F)(F)F)c1C(=O)Nc1ccc(C(=O)N2CC(O)C2)c(Cl)c1] |
| I-241 | 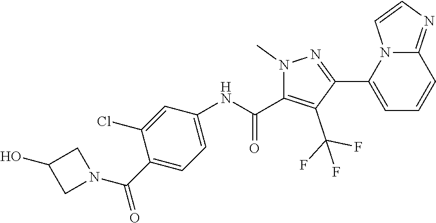 [SMILES: Cn1nc(-c2cccc3nccn23)c(C(F)(F)F)c1C(=O)Nc1ccc(C(=O)N2CC(O)C2)c(Cl)c1] |
| I-242 | 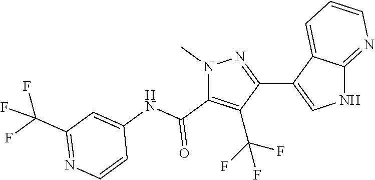 [SMILES: Cn1nc(-c2c[nH]c3ncccc23)c(C(F)(F)F)c1C(=O)Nc1ccnc(C(F)(F)F)c1] |
| I-243 | 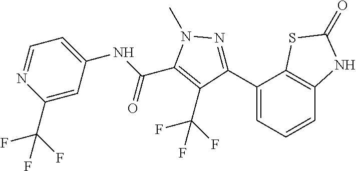 [SMILES: Cn1nc(-c2cccc3[nH]c(=O)sc23)c(C(F)(F)F)c1C(=O)Nc1ccnc(C(F)(F)F)c1] |
| I-244 | 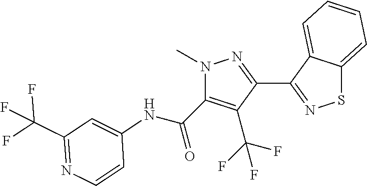 [SMILES: Cn1nc(-c2nsc3ccccc23)c(C(F)(F)F)c1C(=O)Nc1ccnc(C(F)(F)F)c1] |
| I-245 | 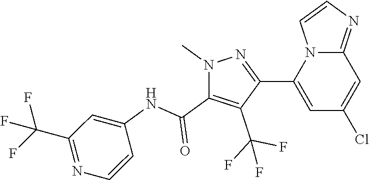 [SMILES: Cn1nc(-c2cc(Cl)cc3nccn23)c(C(F)(F)F)c1C(=O)Nc1ccnc(C(F)(F)F)c1] |
| I-246 | 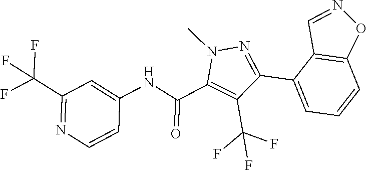 [SMILES: Cn1nc(-c2cccc3oncc23)c(C(F)(F)F)c1C(=O)Nc1ccnc(C(F)(F)F)c1] |
| I-247 | 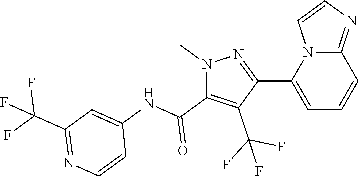 [SMILES: Cn1nc(-c2cccc3nccn23)c(C(F)(F)F)c1C(=O)Nc1ccnc(C(F)(F)F)c1] |
| I-248 | 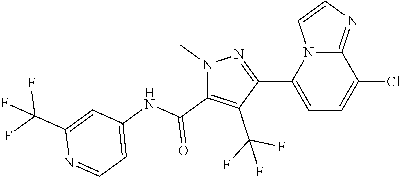 [SMILES: Cn1nc(-c2ccc(Cl)c3nccn23)c(C(F)(F)F)c1C(=O)Nc1ccnc(C(F)(F)F)c1] |
| I-249 | 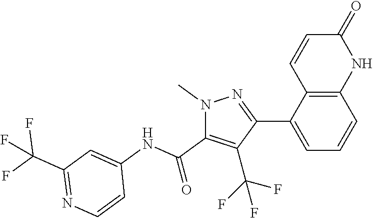 [SMILES: Cn1nc(-c2cccc3[nH]c(=O)ccc23)c(C(F)(F)F)c1C(=O)Nc1ccnc(C(F)(F)F)c1] |
| I-250 | 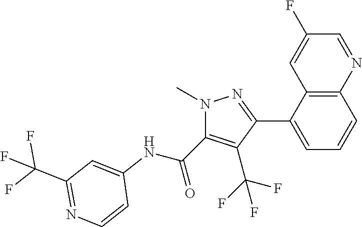 [SMILES: Cn1nc(-c2cccc3ncc(F)cc23)c(C(F)(F)F)c1C(=O)Nc1ccnc(C(F)(F)F)c1] |
| I-251 | 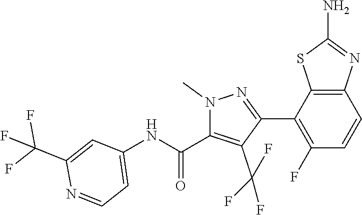 [SMILES: Cn1nc(-c2c(F)ccc3nc(N)sc23)c(C(F)(F)F)c1C(=O)Nc1ccnc(C(F)(F)F)c1] |

Another aspect of the invention provides a compound represented by Formula II:

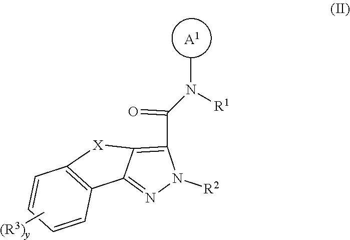

or a pharmaceutically acceptable salt thereof; wherein:
A¹ is a 6 membered heteroaryl containing 1 or 2 nitrogen atoms, wherein the heteroaryl is substituted with m occurrences of R⁵ and n occurrences of R⁶;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, or C₃₋₇ cycloalkyl;
R³ represents independently for each occurrence halo, hydroxyl, C₁₋₆ alkyl, C₁₋₆ haloalkyl, C₁₋₆ alkoxyl, or C₃₋₇ cycloalkyl;
R⁴ and R⁷ are independently hydrogen, C₁₋₆ alkyl, or C₃₋₇ cycloalkyl, or R⁴ and R⁷ are taken together with the nitrogen atom to which they are attached to form a 3-7 membered heterocyclic ring containing 1 nitrogen atom;
R⁵ is a 5-6 membered heteroaryl containing 1, 2, or 3 heteroatoms independently selected from oxygen, nitrogen, and sulfur, wherein the heteroaryl is substituted with q occurrences of R⁸;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, or —N(R⁴)(R⁷);
R⁸ represents independently for each occurrence halo, hydroxyl, C₁₋₆ alkyl, C₁₋₆ haloalkyl, C₁₋₆ alkoxyl, or C₃₋₇ cycloalkyl;
X is a C₁₋₇ bivalent straight or branched saturated hydrocarbon chain wherein 1 or 2 methylene units of the chain are independently and optionally replaced with O; and
m, n, and q are independently 0, 1, or 2.

The definitions of variables in Formula II above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula II.

As defined generally above, A¹ is a 6 membered heteroaryl containing 1 or 2 nitrogen atoms, wherein the heteroaryl is substituted with m occurrences of R⁵ and n occurrences of R⁶. In certain embodiments, A¹ is a 6 membered heteroaryl containing 1 nitrogen atom, wherein the heteroaryl is substituted with m occurrences of R⁵ and n occurrences of R⁶. In certain embodiments, A¹ is a 6 membered heteroaryl containing 2 nitrogen atoms, wherein the heteroaryl is substituted with m occurrences of R⁵ and n occurrences of R⁶. In certain embodiments, A¹ is selected from the groups depicted in the compounds in Table 2 below.

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 2 below.

As defined generally above, R² is C₁₋₆ alkyl, C₁₋₆ haloalkyl, C₁₋₆ deuteroalkyl, C₁₋₆ hydroxyalkyl, or C₃₋₇ cycloalkyl. In some embodiments, R² is C₁₋₆ alkyl. In some embodiments, R² is methyl. In some embodiments, R² is C₁ alkyl. In some embodiments, R² is C₂ alkyl. In some embodiments, R² is C₃ alkyl. In some embodiments, R² is C₄ alkyl. In some embodiments, R² is C₅ alkyl. In some embodiments, R² is C₆ alkyl. In some embodiments, R² is C₁₋₆ haloalkyl. In some embodiments, R² is C₁₋₆ deuteroalkyl. In some embodiments, R² is C₁₋₆ hydroxyalkyl. In some embodiments, R² is C₃₋₇ cycloalkyl. In certain embodiments, R² is selected from the groups depicted in the compounds in Table 2 below.

As defined generally above, R³ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R³ is halo. In certain embodiments, R³ is hydroxyl. In certain embodiments, R³ is C₁₋₆ alkyl. In certain embodiments, R³ is C₁₋₆ haloalkyl. In certain embodiments, R³ is C₁₋₆ alkoxyl. In certain embodiments, R³ is C₃₋₇ cycloalkyl. In certain embodiments, R³ is selected from the groups depicted in the compounds in Table 2 below.

As defined generally above, R⁴ and R⁷ are independently hydrogen, C₁₋₆ alkyl, or C₃₋₇ cycloalkyl, or R⁴ and R⁷ are taken together with the nitrogen atom to which they are attached to form a 3-7 membered heterocyclic ring containing 1 nitrogen atom. In certain embodiments, R⁴ is hydrogen. In certain embodiments, R⁴ is C₁₋₆ alkyl. In certain embodiments, R⁴ is C₃₋₇ cycloalkyl. In certain embodiments, R⁷ is hydrogen. In certain embodiments, R⁷ is C₁₋₆ alkyl. In certain embodiments, R⁷ is C₃₋₇ cycloalkyl. In certain embodiments, R⁴ and R⁷ are taken together with the nitrogen atom to which they are attached to form a 3-7 membered heterocyclic ring containing 1 nitrogen atom. In certain embodiments, R⁴ and R⁷ are taken together with the nitrogen atom to which they are attached to form a 3 membered heterocyclic ring containing 1 nitrogen atom. In certain embodiments, R⁴ and R⁷ are taken together with the nitrogen atom to which they are attached to form a 4 membered heterocyclic ring containing 1 nitrogen atom. In certain embodiments, R⁴ and R⁷ are taken together with the nitrogen atom to which they are attached to form a 5 membered heterocyclic ring containing 1 nitrogen atom. In certain embodiments, R⁴ and R⁷ are taken together with the nitrogen atom to which they are attached to form a 6 membered heterocyclic ring containing 1 nitrogen atom. In certain embodiments, R⁴ and R⁷ are taken together with the nitrogen atom to which they are attached to form a 7 membered heterocyclic ring containing 1 nitrogen atom. In certain embodiments, R⁴ is selected from the groups depicted in the compounds in Table 1 below. In certain embodiments, R⁷ is selected from the groups depicted in the compounds in Table 2 below.

As defined generally above, R⁵ is a 5-6 membered heteroaryl containing 1, 2, or 3 heteroatoms independently selected from oxygen, nitrogen, and sulfur, wherein the heteroaryl is substituted with q occurrences of R⁸. In certain embodiments, R⁵ is a 5 membered heteroaryl containing 1, 2, or 3 heteroatoms independently selected from oxygen, nitrogen, and sulfur, wherein the heteroaryl is substituted with q occurrences of R⁸. In certain embodiments, R⁵ is 1,2,3-triazolyl, pyrazolyl, oxazolyl, imidazolyl, isoxazolyl, pyrrolyl, or furanyl, each of which substituted with q occurrences of R⁸. In certain embodiments, R⁵ is 1,2,3-triazolyl substituted with q occurrences of R⁸. In certain embodiments, R⁵ is a 5 membered heteroaryl containing 1 heteroatom selected from oxygen, nitrogen, and sulfur, wherein the heteroaryl is substituted with q occurrences of R⁸. In certain embodiments, R⁵ is a 5 membered heteroaryl containing 2 heteroatoms independently selected from oxygen, nitrogen, and sulfur, wherein the heteroaryl is substituted with q occurrences of R⁸. In certain embodiments, R⁵ is a 5 membered heteroaryl containing 3 heteroatoms independently selected from oxygen, nitrogen, and sulfur, wherein the heteroaryl is substituted with q occurrences of R⁸. In certain embodiments, R⁵ is a 6 membered heteroaryl containing 1 heteroatom selected from oxygen, nitrogen, and sulfur, wherein the heteroaryl is substituted with q occurrences of R⁸. In certain embodiments, R⁵ is a 6 membered heteroaryl containing 2 heteroatoms independently selected from oxygen, nitrogen, and sulfur, wherein the heteroaryl is substituted with q occurrences of R⁸. In certain embodiments, R⁵ is a 6 membered heteroaryl containing 3 heteroatoms independently selected from oxygen, nitrogen, and sulfur, wherein the heteroaryl is substituted with q occurrences of R⁸. In certain embodiments, R⁵ is pyrazolyl substituted with q occurrences of R⁸. In certain embodiments, R⁵ oxazolyl substituted with q occurrences of R⁸. In certain embodiments, R⁵ is imidazolyl substituted with q occurrences of R⁸. In certain embodiments, R⁵ is isoxazolylsubstituted with q occurrences of R⁸. In certain embodiments, R⁵ is pyrrolylsubstituted with q occurrences of R⁸. In certain embodiments, R⁵ is furanylsubstituted with q occurrences of R⁸. In certain embodiments, R⁵ is selected from the groups depicted in the compounds in Table 2 below.

As defined generally above, R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, or —N(R⁴)(R⁷). In certain embodiments, R⁶ represents independently for each occurrence halo, C₁₋₆ alkyl, or C₁₋₆ haloalkyl. In certain embodiments, R⁶ is chloro. In certain embodiments, R⁶ is C₁₋₆ haloalkyl. In certain embodiments, R⁶ is —CF₃. In certain embodiments, R⁶ is halo. In certain embodiments, R⁶ is fluoro. In certain embodiments, R⁶ is bromo. In certain embodiments, R⁶ is iodo. In certain embodiments, R⁶ is hydroxyl. In certain embodiments, R⁶ is C₁₋₆ alkyl. In certain embodiments, R⁶ is C₁₋₆ hydroxyalkyl. In certain embodiments, R⁶ is C₁₋₆ alkoxyl. In certain embodiments, R⁶ is C₃₋₇ cycloalkyl. In certain embodiments, R⁶ is cyano. In certain embodiments, R⁶ is —O—C₃₋₇ cycloalkyl. In certain embodiments, R⁶ is —N(R⁴)(R⁷). In certain embodiments, R⁶ is selected from the groups depicted in the compounds in Table 2 below.

As defined generally above, R⁸ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R⁸ is halo. In certain embodiments, R⁸ is F. In certain embodiments, R⁸ is C₁. In certain embodiments, R⁸ is Br. In certain embodiments, R⁸ is I. In certain embodiments, R⁸ is hydroxyl. In certain embodiments, R⁸ is C₁₋₆ alkyl. In certain embodiments, R⁸ is C₁₋₆ haloalkyl. In certain embodiments, R⁸ is C₁₋₆ alkoxyl. In certain embodiments, R⁸ is C₃₋₇ cycloalkyl. In certain embodiments, R⁸ is selected from the groups depicted in the compounds in Table 2 below.

As defined generally above, X is a C₁₋₇ bivalent straight or branched saturated hydrocarbon chain wherein 1 or 2 methylene units of the chain are independently and optionally replaced with O. In certain embodiments, X is —CH₂CH₂— or —CH₂CH₂CH₂—. In certain embodiments, X is —CH₂CH₂CH₂— wherein one CH₂ is replaced with —O—. In certain embodiments, X is —CH₂CH₂—. In certain embodiments, X is —CH₂CH₂CH₂—. In certain embodiments, X is a C₁ bivalent straight or branched saturated hydrocarbon chain wherein the 1 methylene unit of the chain is optionally replaced with O. In certain embodiments, X is a C₂ bivalent straight or branched saturated hydrocarbon chain wherein 1 or 2 methylene units of the chain are independently and optionally replaced with O. In certain embodiments, X is a C₃ bivalent straight or branched saturated hydrocarbon chain wherein 1 or 2 methylene units of the chain are independently and optionally replaced with O. In certain embodiments, X is a C₄ bivalent straight or branched saturated hydrocarbon chain wherein 1 or 2 methylene units of the chain are independently and optionally replaced with O. In certain embodiments, X is a C₅ bivalent straight or branched saturated hydrocarbon chain wherein 1 or 2 methylene units of the chain are independently and optionally replaced with O. In certain embodiments, X is a C₆ bivalent straight or branched saturated hydrocarbon chain wherein 1 or 2 methylene units of the chain are independently and optionally replaced with O. In certain embodiments, X is a C₇ bivalent straight or branched saturated hydrocarbon chain wherein 1 or 2 methylene units of the chain are independently and optionally replaced with O. In certain embodiments, X is selected from the groups depicted in the compounds in Table 2 below.

As defined generally above, m, n, and q are independently 0, 1, or 2. In certain embodiments, q is 0. In certain embodiments, m is 1. In certain embodiments, m is 0. In certain embodiments, n is 1. In certain embodiments, m is 2. In certain embodiments, n is 0. In certain embodiments, n is 2. In certain embodiments, q is 1. In certain embodiments, q is 2. In certain embodiments, m is selected from the corresponding value in the groups depicted in the compounds in Table 2 below. In certain embodiments, n is selected from the corresponding value in the groups depicted in the compounds in Table 2 below. In certain embodiments, q is selected from the corresponding value in the groups depicted in the compounds in Table 2 below.

The description above describes multiple embodiments relating to compounds of Formula II. The patent application specifically contemplates all combinations of the embodiments.

Another aspect of the invention provides a compound in Table 2 below, or a pharmaceutically acceptable salt thereof. In certain embodiments, the compound is a compound in Table 2.

TABLE 2

| Com- | |
|---|---|
| pound | |
| No. | Chemical Structure |
| I-252 | 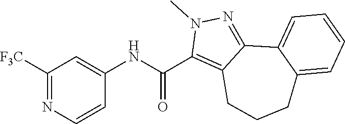 [SMILES: Cn1nc2c(c1C(=O)Nc1ccnc(C(F)(F)F)c1)CCCc1ccccc1-2] |
| I-253 | 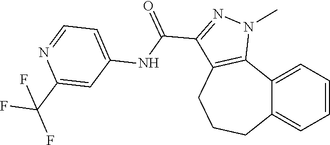 [SMILES: Cn1nc(C(=O)Nc2ccnc(C(F)(F)F)c2)c2c1-c1ccccc1CCC2] |
| I-254 | 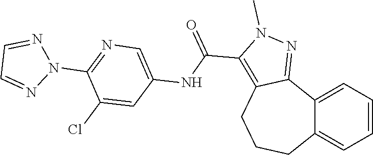 [SMILES: Cn1nc2c(c1C(=O)Nc1cnc(-n3nccn3)c(Cl)c1)CCCc1ccccc1-2] |
| I-255 | 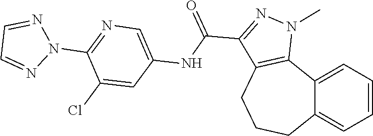 [SMILES: Cn1nc(C(=O)Nc2cnc(-n3nccn3)c(Cl)c2)c2c1-c1ccccc1CCC2] |
| I-256 | 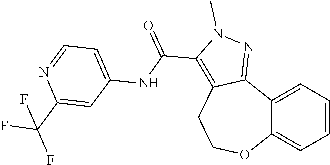 [SMILES: Cn1nc2c(c1C(=O)Nc1ccnc(C(F)(F)F)c1)CCOc1ccccc1-2] |
| I-257 | 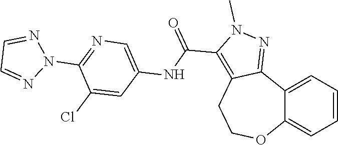 [SMILES: Cn1nc2c(c1C(=O)Nc1cnc(-n3nccn3)c(Cl)c1)CCOc1ccccc1-2] |
| I-258 | 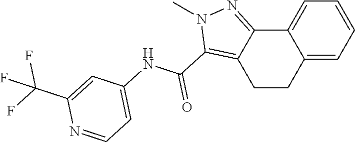 [SMILES: Cn1nc2c(c1C(=O)Nc1ccnc(C(F)(F)F)c1)CCc1ccccc1-2] |
| I-259 | 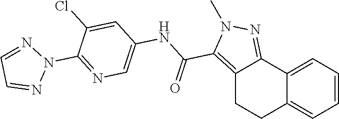 [SMILES: Cn1nc2c(c1C(=O)Nc1cnc(-n3nccn3)c(Cl)c1)CCc1ccccc1-2] |
| I-260 | 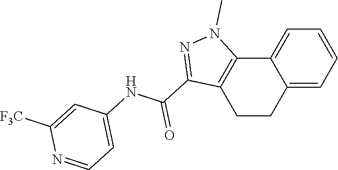 [SMILES: Cn1nc(C(=O)Nc2ccnc(C(F)(F)F)c2)c2c1-c1ccccc1CC2] |

Another aspect of the invention provides a compound represented by Formula III:

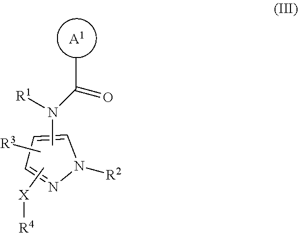

or a pharmaceutically acceptable salt thereof, wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur,
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

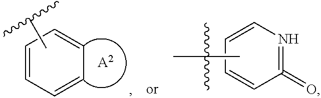

wherein the phenyl, heteroaryl,

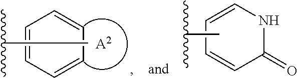

are substituted with t occurrences of R⁷;
R⁵ is a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹;
R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—; and m, n, q, t, and y are independently 0, 1, or 2;
provided that R² and R³ are not simultaneously C₁₋₄ alkyl.

The definitions of variables in Formula III above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula III.

As defined generally above, A¹ is phenyl or a 6 membered heteroaryl containing 1 or 2 nitrogen atoms, wherein the phenyl and heteroaryl are substituted with m occurrences of R⁵ and n occurrences of R⁶. In certain embodiments, A¹ is pyridinyl substituted with m occurrences of R⁵ and n occurrences of R⁶. In certain embodiments, A¹ is phenyl substituted with m occurrences of R⁵ and n occurrences of R⁶. In certain embodiments, A¹ is a 6 membered heteroaryl containing 1 nitrogen atom, wherein the heteroaryl is substituted with m occurrences of R⁵ and n occurrences of R⁶. A¹ is a 6 membered heteroaryl containing 2 nitrogen atoms, wherein the heteroaryl is substituted with m occurrences of R⁵ and n occurrences of R⁶. In certain embodiments, A¹ is selected from the groups depicted in the compounds in Table 3 below.

As defined generally above, A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur. In certain embodiments, A² is a 5-7 membered partially unsaturated ring containing 0, 1, 2, or 3 heteroatoms independently selected from oxygen, nitrogen, and sulfur. In certain embodiments, A² is a 5 membered partially unsaturated ring containing 0, 1, 2, or 3 heteroatoms independently selected from oxygen, nitrogen, and sulfur. In certain embodiments, A² is a 5 membered partially unsaturated ring containing 0 heteroatoms. In certain embodiments, A² is a 5 membered partially unsaturated ring containing 1 heteroatom selected from oxygen, nitrogen, and sulfur. In certain embodiments, A² is a 5 membered partially unsaturated ring containing 2 heteroatoms independently selected from oxygen, nitrogen, and sulfur. In certain embodiments, A² is a 5 membered partially unsaturated ring containing 3 heteroatoms independently selected from oxygen, nitrogen, and sulfur. In certain embodiments, A² is a 6 membered partially unsaturated ring containing 0, 1, 2, or 3 heteroatoms independently selected from oxygen, nitrogen, and sulfur. In certain embodiments, A² is a 6 membered partially unsaturated ring containing 0 heteroatoms. In certain embodiments, A² is a 6 membered partially unsaturated ring containing 1 heteroatom selected from oxygen, nitrogen, and sulfur. In certain embodiments, A² is a 6 membered partially unsaturated ring containing 2 heteroatoms independently selected from oxygen, nitrogen, and sulfur. In certain embodiments, A² is a 6 membered partially unsaturated ring containing 3 heteroatoms independently selected from oxygen, nitrogen, and sulfur. In certain embodiments, A² is a 7 membered partially unsaturated ring containing 0, 1, 2, or 3 heteroatoms independently selected from oxygen, nitrogen, and sulfur. In certain embodiments, A² is a 7 membered partially unsaturated ring containing 0 heteroatoms. In certain embodiments, A² is a 7 membered partially unsaturated ring containing 1 heteroatom selected from oxygen, nitrogen, and sulfur. In certain embodiments, A² is a 7 membered partially unsaturated ring containing 2 heteroatoms independently selected from oxygen, nitrogen, and sulfur. In certain embodiments, A² is a 7 membered partially unsaturated ring containing 3 heteroatoms independently selected from oxygen, nitrogen, and sulfur. In certain embodiments, A² is a 5-7 membered partially unsaturated oxo-substituted ring containing 0, 1, 2, or 3 heteroatoms independently selected from oxygen, nitrogen, and sulfur. In certain embodiments, A² is a 5 membered partially unsaturated oxo-substituted ring containing 0, 1, 2, or 3 heteroatoms independently selected from oxygen, nitrogen, and sulfur. In certain embodiments, A² is a 5 membered partially unsaturated oxo-substituted ring containing 0 heteroatoms. In certain embodiments, A² is a 5 membered partially unsaturated oxo-substituted ring containing 1 heteroatom selected from oxygen, nitrogen, and sulfur. In certain embodiments, A² is a 5 membered partially unsaturated oxo-substituted ring containing 2 heteroatoms independently selected from oxygen, nitrogen, and sulfur. In certain embodiments, A² is a 5 membered partially unsaturated oxo-substituted ring containing 3 heteroatoms independently selected from oxygen, nitrogen, and sulfur. In certain embodiments, A² is a 6 membered partially unsaturated oxo-substituted ring containing 0, 1, 2, or 3 heteroatoms independently selected from oxygen, nitrogen, and sulfur. In certain embodiments, A² is a 6 membered partially unsaturated oxo-substituted ring containing 0 heteroatoms. In certain embodiments, A² is a 6 membered partially unsaturated oxo-substituted ring containing 1 heteroatom selected from oxygen, nitrogen, and sulfur. In certain embodiments, A² is a 6 membered partially unsaturated oxo-substituted ring containing 2 heteroatoms independently selected from oxygen, nitrogen, and sulfur. In certain embodiments, A² is a 6 membered partially unsaturated oxo-substituted ring containing 3 heteroatoms independently selected from oxygen, nitrogen, and sulfur. In certain embodiments, A² is a 7 membered partially unsaturated oxo-substituted ring containing 0, 1, 2, or 3 heteroatoms independently selected from oxygen, nitrogen, and sulfur. In certain embodiments, A² is a 7 membered partially unsaturated oxo-substituted ring containing 0 heteroatoms. In certain embodiments, A² is a 7 membered partially unsaturated oxo-substituted ring containing 1 heteroatom selected from oxygen, nitrogen, and sulfur. In certain embodiments, A² is a 7 membered partially unsaturated oxo-substituted ring containing 2 heteroatoms independently selected from oxygen, nitrogen, and sulfur. In certain embodiments, A² is a 7 membered partially unsaturated oxo-substituted ring containing 3 heteroatoms independently selected from oxygen, nitrogen, and sulfur. In certain embodiments, A² is selected from the groups depicted in the compounds in Table 3 below.

As defined generally above, A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 4 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 4 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 4 membered saturated heterocyclyl containing 2 heteroatoms independently selected from oxygen, nitrogen, and sulfur, wherein the heterocyclyl is substituted with y occurrences of R¹². In certain embodiments, A³ represents independently for each occurrence a 5 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 5 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 5 membered saturated heterocyclyl containing 2 heteroatoms independently selected from oxygen, nitrogen, and sulfur, wherein the heterocyclyl is substituted with y occurrences of R¹². In certain embodiments, A³ represents independently for each occurrence a 6 membered saturated heterocyclyl containing 1 or 2 heteroatoms independently selected from oxygen, nitrogen, and sulfur, wherein the heterocyclyl is substituted with y occurrences of R¹². In certain embodiments, A³ represents independently for each occurrence a 6 membered saturated heterocyclyl containing 1 heteroatom selected from oxygen, nitrogen, and sulfur, wherein the heterocyclyl is substituted with y occurrences of R¹². In certain embodiments, A³ represents independently for each occurrence a 6 membered saturated heterocyclyl containing 2 heteroatoms independently selected from oxygen, nitrogen, and sulfur, wherein the heterocyclyl is substituted with y occurrences of R¹². In certain embodiments, A³ is selected from the groups depicted in the compounds in Table 3 below.

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 3 below.

As defined generally above, R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms: or R² and one occurrence of R⁷ are taken together with their intervening atoms to form a 5-7 membered ring containing 1 nitrogen atom. In certain embodiments, R² is C₁₋₆ alkyl. In certain embodiments, R² is methyl. In certain embodiments, R² is C₁₋₄ haloalkyl. In certain embodiments, R² is C₁₋₆ deuteroalkyl. In certain embodiments, R² is C₁₋₆ hydroxyalkyl. In certain embodiments, R² is C₃₋₇ cycloalkyl. In certain embodiments, R² is —(C₁₋₆ alkylene)-N(R⁸)(R⁹). In certain embodiments, R² is —(C₁₋₆ alkylene)-C₁₋₆ alkoxyl. In certain embodiments, R² is a 3-5 membered saturated heterocyclyl containing 1 or 2 heteroatoms independently selected from oxygen and nitrogen. In certain embodiments, R² is a 3 membered saturated heterocyclyl containing 1 or 2 heteroatoms independently selected from oxygen and nitrogen. In certain embodiments, R² is a 3 membered saturated heterocyclyl containing 1 heteroatom selected from oxygen and nitrogen. In certain embodiments, R² is a 3 membered saturated heterocyclyl containing 2 heteroatoms independently selected from oxygen and nitrogen. In certain embodiments, R² is a 4 membered saturated heterocyclyl containing 1 or 2 heteroatoms independently selected from oxygen and nitrogen. In certain embodiments, R² is a 4 membered saturated heterocyclyl containing 1 heteroatom selected from oxygen and nitrogen. In certain embodiments, R² is a 4 membered saturated heterocyclyl containing 2 heteroatoms independently selected from oxygen and nitrogen. In certain embodiments, R² is a 5 membered saturated heterocyclyl containing 1 or 2 heteroatoms independently selected from oxygen and nitrogen. In certain embodiments, R² is a 5 membered saturated heterocyclyl containing 1 heteroatom selected from oxygen and nitrogen. In certain embodiments, R² is a 5 membered saturated heterocyclyl containing 2 heteroatoms independently selected from oxygen and nitrogen. In certain embodiments, R² and R¹ are taken together with their intervening atoms to form a 5-7 membered ring containing 2 nitrogen atoms. In certain embodiments, R² and R¹ are taken together with their intervening atoms to form a 5 membered ring containing 2 nitrogen atoms. In certain embodiments, R² and R¹ are taken together with their intervening atoms to form a 6 membered ring containing 2 nitrogen atoms. In certain embodiments, R² and R¹ are taken together with their intervening atoms to form a 7 membered ring containing 2 nitrogen atoms. In certain embodiments, R² and one occurrence of R⁷ are taken together with their intervening atoms to form a 5-7 membered ring containing 1 nitrogen atom. In certain embodiments, R² and one occurrence of R⁷ are taken together with their intervening atoms to form a 5 membered ring containing 1 nitrogen atom. In certain embodiments, R² and one occurrence of R⁷ are taken together with their intervening atoms to form a 6 membered ring containing 1 nitrogen atom. In certain embodiments, R² and one occurrence of R⁷ are taken together with their intervening atoms to form a 7 membered ring containing 1 nitrogen atom. In certain embodiments, R² is selected from the groups depicted in the compounds in Table 3 below.

As defined generally above, R³ is C₁₋₆ haloalkyl, C₁₋₆ alkyl, or hydrogen. In certain embodiments, R³ is C₁₋₆ haloalkyl. In certain embodiments, R³ is —CF₃. In certain embodiments, R³ is C₁₋₆ alkyl. In certain embodiments, R³ is methyl. In certain embodiments, R³ is C₂ alkyl. In certain embodiments, R³ is C₃ alkyl. In certain embodiments, R³ is C₄ alkyl. In certain embodiments, R³ is C₅ alkyl. In certain embodiments, R³ is C₆ alkyl. In certain embodiments, R³ is hydrogen. In certain embodiments, R³ is selected from the groups depicted in the compounds in Table 3 below.

As defined generally above, R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

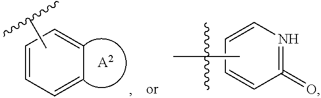

wherein the phenyl, heteroaryl.

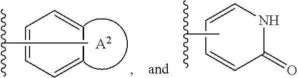

are substituted with t occurrences of R⁷. In certain embodiments, R⁴ is phenyl substituted with t occurrences of R⁷. In certain embodiments, R⁴ is a 5-6 membered heteroaryl containing 1, 2, or 3 heteroatoms independently selected from oxygen, nitrogen, and sulfur, wherein the heteroaryl is substituted with t occurrences of R⁷. In certain embodiments, R⁴ is pyridinyl substituted with t occurrences of R⁷. In certain embodiments, R⁴ is a 9-10 membered bicyclic heteroaryl containing 1, 2, or 3 heteroatoms independently selected from oxygen, nitrogen, and sulfur, wherein the heteroaryl is substituted with t occurrences of R⁷. In certain embodiments, R⁴ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is

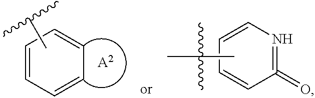

each of which are substituted with t occurrences of R⁷. In certain embodiments, R⁴ is a 5 membered heteroaryl containing 1, 2, or 3 heteroatoms independently selected from oxygen, nitrogen, and sulfur, wherein the heteroaryl is substituted with t occurrences of R⁷. In certain embodiments, R⁴ is a 5 membered heteroaryl containing 1 heteroatom selected from oxygen, nitrogen, and sulfur, wherein the heteroaryl is substituted with t occurrences of R⁷. In certain embodiments, R⁴ is a 5 membered heteroaryl containing 2 heteroatoms independently selected from oxygen, nitrogen, and sulfur, wherein the heteroaryl is substituted with t occurrences of R⁷. In certain embodiments, R⁴ is a 5 membered heteroaryl containing 3 heteroatoms independently selected from oxygen, nitrogen, and sulfur, wherein the heteroaryl is substituted with t occurrences of R⁷. In certain embodiments, R⁴ is a 6 membered heteroaryl containing 1, 2, or 3 heteroatoms independently selected from oxygen, nitrogen, and sulfur, wherein the heteroaryl is substituted with t occurrences of R⁷. In certain embodiments, R⁴ is a 6 membered heteroaryl containing 1 heteroatom selected from oxygen, nitrogen, and sulfur, wherein the heteroaryl is substituted with t occurrences of R⁷. In certain embodiments, R⁴ is a 6 membered heteroaryl containing 2 heteroatoms independently selected from oxygen, nitrogen, and sulfur, wherein the heteroaryl is substituted with t occurrences of R⁷. In certain embodiments, R⁴ is a 6 membered heteroaryl containing 3 heteroatoms independently selected from oxygen, nitrogen, and sulfur, wherein the heteroaryl is substituted with t occurrences of R⁷. In certain embodiments, R⁴ is a 9 membered bicyclic heteroaryl containing 1, 2, or 3 heteroatoms independently selected from oxygen, nitrogen, and sulfur, wherein the heteroaryl is substituted with t occurrences of R⁷. In certain embodiments, R⁴ is a 9 membered bicyclic heteroaryl containing 1 heteroatom independently selected from oxygen, nitrogen, and sulfur, wherein the heteroaryl is substituted with t occurrences of R⁷. In certain embodiments, R⁴ is a 9 membered bicyclic heteroaryl containing 2 heteroatoms independently selected from oxygen, nitrogen, and sulfur, wherein the heteroaryl is substituted with t occurrences of R⁷. In certain embodiments, R⁴ is a 9 membered bicyclic heteroaryl containing 3 heteroatoms independently selected from oxygen, nitrogen, and sulfur, wherein the heteroaryl is substituted with t occurrences of R⁷. In certain embodiments, R⁴ is a 10 membered bicyclic heteroaryl containing 1, 2, or 3 heteroatoms independently selected from oxygen, nitrogen, and sulfur, wherein the heteroaryl is substituted with t occurrences of R⁷. In certain embodiments, R⁴ is a 10 membered bicyclic heteroaryl containing 1 heteroatom independently selected from oxygen, nitrogen, and sulfur, wherein the heteroaryl is substituted with t occurrences of R⁷. In certain embodiments, R⁴ is a 10 membered bicyclic heteroaryl containing 2 heteroatoms independently selected from oxygen, nitrogen, and sulfur, wherein the heteroaryl is substituted with t occurrences of R⁷. In certain embodiments, R⁴ is a 10 membered bicyclic heteroaryl containing 3 heteroatoms independently selected from oxygen, nitrogen, and sulfur, wherein the heteroaryl is substituted with t occurrences of R⁷. In certain embodiments, R⁴ is a 3 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 3 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 4 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 5 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 6 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 7 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 8 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is an 8 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 9 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 1 heteroatom selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 2 heteroatoms independently selected from oxygen, nitrogen, and sulfur, wherein the heterocyclyl is substituted with t occurrences of R⁷. In certain embodiments, R⁴ is a 10 membered monocyclic or bicyclic saturated or partially unsaturated heterocyclyl containing 3 heteroatoms independently selected from oxygen, nitrogen, and sulfur, wherein the heterocyclyl is substituted with t occurrences of R⁷. In certain embodiments, R⁴ is

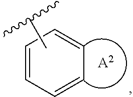

which is substituted with t occurrences of R⁷. In certain embodiments, R⁴ is

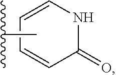

which is substituted with t occurrences of R⁷. In certain embodiments, R⁴ is selected from the groups depicted in the compounds in Table 3 below.

As defined generally above, R⁵ is a 5-6 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 5 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 1,2,3-triazolyl, pyrazolyl, oxazolyl, imidazolyl, isoxazolyl, pyrrolyl, or furanyl, each of which substituted with q occurrences of R¹². In certain embodiments, R⁵ is a 1,2,3-triazolyl substituted with q occurrences of R¹². In certain embodiments, R⁵ is a 5 membered heteroaryl containing 1 heteroatom selected from oxygen, nitrogen, and sulfur, wherein the heteroaryl is substituted with q occurrences of R¹². In certain embodiments, R⁵ is a 5 membered heteroaryl containing 2 heteroatoms independently selected from oxygen, nitrogen, and sulfur, wherein the heteroaryl is substituted with q occurrences of R¹². In certain embodiments, R⁵ is a 5 membered heteroaryl containing 3 heteroatoms independently selected from oxygen, nitrogen, and sulfur, wherein the heteroaryl is substituted with q occurrences of R¹². In certain embodiments, R⁵ is a 6 membered heteroaryl containing 1, 2, or 3 heteroatoms independently selected from oxygen, nitrogen, and sulfur, wherein the heteroaryl is substituted with q occurrences of R¹². In certain embodiments, R⁵ is a 6 membered heteroaryl containing 1 heteroatom selected from oxygen, nitrogen, and sulfur, wherein the heteroaryl is substituted with q occurrences of R¹². In certain embodiments, R⁵ is a 6 membered heteroaryl containing 2 heteroatoms independently selected from oxygen, nitrogen, and sulfur, wherein the heteroaryl is substituted with q occurrences of R¹². In certain embodiments, R⁵ is a 6 membered heteroaryl containing 3 heteroatoms independently selected from oxygen, nitrogen, and sulfur, wherein the heteroaryl is substituted with q occurrences of R¹². In certain embodiments, R⁵ is a pyrazolyl substituted with q occurrences of R¹². In certain embodiments, R⁵ is an oxazolyl substituted with q occurrences of R¹². In certain embodiments, R⁵ is an imidazolyl substituted with q occurrences of R¹². In certain embodiments, R⁵ is an isoxazolyl substituted with q occurrences of R¹². In certain embodiments, R⁵ is a pyrrolyl substituted with q occurrences of R¹². In certain embodiments, R⁵ is a furanyl substituted with q occurrences of R¹². In certain embodiments, R⁵ is selected from the groups depicted in the compounds in Table 3 below.

As defined generally above, R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹. In certain embodiments, R⁶ represents independently for each occurrence halo, C₁₋₆ alkyl, or C₁₋₆ haloalkyl. In certain embodiments, R⁶ is chloro. In certain embodiments, R⁶ is C₁₋₆ haloalkyl. In certain embodiments, R⁶ is —CF₃. In certain embodiments, R⁶ is halo. In certain embodiments, R⁶ is F. In certain embodiments, R⁶ is Br. In certain embodiments, R⁶ is I. In certain embodiments, R⁶ is hydroxyl. In certain embodiments, R⁶ is C₁₋₆ alkyl. In certain embodiments, R⁶ is C₁ alkyl. In certain embodiments, R⁶ is C₂ alkyl. In certain embodiments, R⁶ is C₃ alkyl. In certain embodiments, R⁶ is C₄ alkyl. In certain embodiments, R⁶ is C₅ alkyl. In certain embodiments, R⁶ is C₆ alkyl. In certain embodiments, R⁶ is C₁₋₆ hydroxyalkyl. In certain embodiments, R⁶ is C₁₋₆ alkoxyl. In certain embodiments, R⁶ is C₃₋₇ cycloalkyl. In certain embodiments, R⁶ is cyano. In certain embodiments, R⁶ is —O—C₃₋₇ cycloalkyl. In certain embodiments, R⁶ is —N(R⁹)(R¹⁰). In certain embodiments, R⁶ is —(C₀₋₄ alkylene)-C(O)R⁸. In certain embodiments, R⁶ is —C(O)N(R⁹)(R¹⁰). In certain embodiments, R⁶ is —N(R⁹)C(O)R¹¹. In certain embodiments, R⁶ is —SO₂R¹¹. In certain embodiments, R⁶ is selected from the groups depicted in the compounds in Table 3 below.

As defined generally above, R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³. In certain embodiments, R¹ represents independently for each occurrence halo, hydroxyl, C₁₋₆ alkyl, C₁₋₆ haloalkyl, C₁₋₆ hydroxyalkyl, C₁₋₆ alkoxyl, or C₃₋₇ cycloalkyl. In certain embodiments, R⁷ represents independently for each occurrence halo, C₁₋₆ alkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, or —N(R⁹)(R¹⁰). In certain embodiments, R⁷ represents independently for each occurrence halo, C₁₋₆ alkyl, —(C₀₋₄ alkylene)-C(O)R⁸, —C(O)N(R⁹)(R¹⁰), —N(R⁹)C(O)R¹¹, —SO₂R¹¹, or —O-A³. In certain embodiments, R⁷ is halo. In certain embodiments, R⁷ is hydroxyl. In certain embodiments, R⁷ is C₁₋₆ alkyl. In certain embodiments, R⁷ is C₁₋₆ haloalkyl. In certain embodiments, R⁷ is C₁₋₆ hydroxyalkyl. In certain embodiments, R⁷ is C₁₋₆ alkoxyl. In certain embodiments, R⁷ is C₃₋₇ cycloalkyl. In certain embodiments, R⁷ is cyano. In certain embodiments, RT is —O—C₃₋₇ cycloalkyl. In certain embodiments, R⁷ is —(C₀₋₄ alkylene)-CN. In certain embodiments, R⁷ is —(C₁₋₄ alkylene)-(C₁₋₆ alkoxyl). In certain embodiments, R⁷ is C₂₋₄ alkynyl. In certain embodiments, R⁷ is —N(R⁹)(R¹⁰). In certain embodiments, R⁷ is —(C₀₋₄ alkylene)-C(O)R⁸. In certain embodiments, R⁷ is —C(O)N(R⁹)(R¹⁰). In certain embodiments, R⁷ is —N(R⁹)C(O)R¹¹. In certain embodiments, R⁷ is —SO₂R¹¹. In certain embodiments, R⁷ is —O-A³. In certain embodiments, R⁷ is selected from the groups depicted in the compounds in Table 3 below.

As defined generally above, R⁸ is —OH, —O—(C₁₋₆ alkyl), —O—C₃₋₇ cycloalkyl, or A³. In certain embodiments, R⁸ is —OH. In certain embodiments, R⁸ is —O—(C₁₋₆ alkyl). In certain embodiments, R⁸ is —O—C₃₋₇ cycloalkyl. In certain embodiments, R⁸ is A³. In certain embodiments, R⁸ is selected from the groups depicted in the compounds in Table 3 below.

As defined generally above, R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments. R⁹ is hydrogen. In certain embodiments, R⁹ is C₁₋₆ alkyl. In certain embodiments, R⁹ is C₁ alkyl. In certain embodiments, R⁹ is C₂ alkyl. In certain embodiments, R⁹ is C₃ alkyl. In certain embodiments, R⁹ is C₄ alkyl. In certain embodiments, R⁹ is C₅ alkyl. In certain embodiments, R⁹ is C₆ alkyl. In certain embodiments, R⁹ is C₃₋₇ cycloalkyl. In certain embodiments, R⁹ is C₃ cycloalkyl. In certain embodiments, R⁹ is C₄ cycloalkyl. In certain embodiments, R⁹ is C₅ cycloalkyl. In certain embodiments, R⁹ is C₆ cycloalkyl. In certain embodiments, R⁹ is C₇ cycloalkyl. In certain embodiments, R¹⁰ is hydrogen. In certain embodiments, R¹⁰ is C₁₋₆ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹⁰ is C₅ alkyl. In certain embodiments, R¹⁰ is C₆ alkyl. In certain embodiments, R¹⁰ is C₃₋₇ cycloalkyl. In certain embodiments, R¹⁰ is C₃ cycloalkyl. In certain embodiments, R¹⁰ is C₄ cycloalkyl. In certain embodiments, R¹⁰ is C₅ cycloalkyl. In certain embodiments, R¹⁰ is C₆ cycloalkyl. In certain embodiments, R¹⁰ is C₇ cycloalkyl. In certain embodiments, R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 3 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 4 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 5 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form a 6 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰) are taken together with the nitrogen atom to which they are attached to form a 7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is selected from the groups depicted in the compounds in Table 1 below. In certain embodiments, R¹⁰ is selected from the groups depicted in the compounds in Table 3 below.

As defined generally above, R¹¹ represents independently for each occurrence C₁₋₆ alkyl or (C₀₋₅ alkylene)-C₃₋₇ cycloalkyl. In certain embodiments, R¹¹ is C₁₋₆ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹¹ is C₅ alkyl. In certain embodiments, R¹¹ is C₆ alkyl. In certain embodiments, R¹¹ is (C₀₋₅ alkylene)-C₃₋₇ cycloalkyl. In certain embodiments, R¹¹ is selected from the groups depicted in the compounds in Table 3 below.

As defined generally above, R¹² represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹² is halo. In certain embodiments, R¹² is F. In certain embodiments, R¹² is C₁. In certain embodiments, R¹² is Br. In certain embodiments, R¹² is I. In certain embodiments, R¹² is hydroxyl. In certain embodiments, R¹² is C₁₋₆ alkyl. In certain embodiments, R¹² is C₁ alkyl. In certain embodiments, R¹² is C₂ alkyl. In certain embodiments, R¹² is C₃ alkyl. In certain embodiments, R¹² is C₄ alkyl. In certain embodiments, R¹² is C₅ alkyl. In certain embodiments, R¹² is C₆ alkyl. In certain embodiments, R¹² is C₁₋₆ haloalkyl. In certain embodiments, R¹² is C₁₋₆ alkoxyl. In certain embodiments, R¹² is C₃₋₇ cycloalkyl. In certain embodiments, R¹² is selected from the groups depicted in the compounds in Table 3 below.

As defined generally above, X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—. In certain embodiments, X is a bond. In certain embodiments, X is C₁₋₅ alkylene or C₃₋₅ cycloalkylene. In certain embodiments, X is C₂₋₄ alkenylene, —(C₀₋₂ alkylene)-O—(C₀₋₂ alkylene), or —(C₀₋₂ alkylene)-N(R⁹)—(C₀₋₂ alkylene). In certain embodiments, X is C₁₋₅ alkylene. In certain embodiments, X is C₃₋₅ cycloalkylene. In certain embodiments, X is C₂₋₄ alkenylene. In certain embodiments, X is —(C₀₋₂ alkylene)-O—(C₀₋₂ alkylene). In certain embodiments, X is —(C₀₋₂ alkylene)-N(R⁹)—(C₀₋₂ alkylene). In certain embodiments, X is —C(O)—. In certain embodiments, X is selected from the groups depicted in the compounds in Table 3 below.

As defined generally above, m, n, q, t, and y are independently 0, 1, or 2. In certain embodiments, t is 1. In certain embodiments, t is 0. In certain embodiments, q is 0. In certain embodiments, m is 0. In certain embodiments, m is 1. In certain embodiments, m is 2. In certain embodiments, n is 0. In certain embodiments, n is 1. In certain embodiments, n is 2. In certain embodiments, q is 1. In certain embodiments, q is 2. In certain embodiments, t is 2. In certain embodiments, y is 0. In certain embodiments, y is 1. In certain embodiments, y is 2. In certain embodiments, m is selected from the corresponding value in the groups depicted in the compounds in Table 1 below. In certain embodiments, n is selected from the corresponding value in the groups depicted in the compounds in Table 1 below. In certain embodiments, q is selected from the corresponding value in the groups depicted in the compounds in Table 1 below. In certain embodiments, t is selected from the corresponding value in the groups depicted in the compounds in Table 1 below. In certain embodiments, y is selected from the corresponding value in the groups depicted in the compounds in Table 3 below.

The description above describes multiple embodiments relating to compounds of Formula III. The patent application specifically contemplates all combinations of the embodiments.

Another aspect of the invention provides a compound in Table 3 below, or a pharmaceutically acceptable salt thereof. In certain embodiments, the compound is a compound in Table 3. In certain embodiments, the compound is any one of compounds I-221 or I-261 to I-279 in Table 3, or a pharmaceutically acceptable salt thereof. In certain embodiments, the compound is any one of compounds I-221 or I-261 to I-279 in Table 3.

TABLE 3

| Compound | |
|---|---|
| No. | Chemical Structure |
| I-221 | 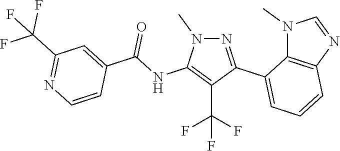 [SMILES: Cn1nc(-c2cccc3ncn(C)c23)c(C(F)(F)F)c1NC(=O)c1ccnc(C(F)(F)F)c1] |
| I-261 | 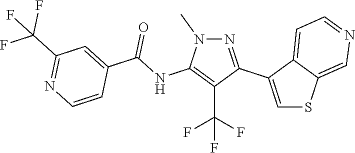 [SMILES: Cn1nc(-c2csc3cnccc23)c(C(F)(F)F)c1NC(=O)c1ccnc(C(F)(F)F)c1] |
| I-262 | 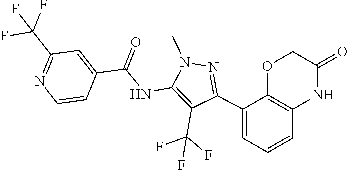 [SMILES: Cn1nc(-c2cccc3c2OCC(=O)N3)c(C(F)(F)F)c1NC(=O)c1ccnc(C(F)(F)F)c1] |
| I-263 | 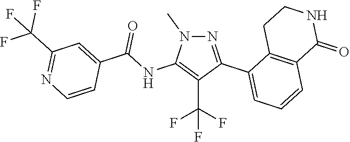 [SMILES: Cn1nc(-c2cccc3c2CCNC3=O)c(C(F)(F)F)c1NC(=O)c1ccnc(C(F)(F)F)c1] |
| I-264 | 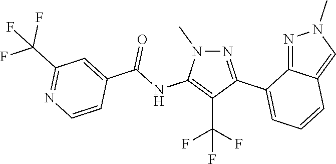 [SMILES: Cn1cc2cccc(-c3nn(C)c(NC(=O)c4ccnc(C(F)(F)F)c4)c3C(F)(F)F)c2n1] |
| I-265 | 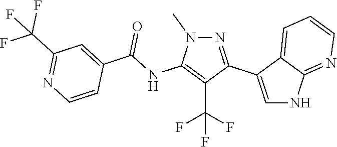 [SMILES: Cn1nc(-c2c[nH]c3ncccc23)c(C(F)(F)F)c1NC(=O)c1ccnc(C(F)(F)F)c1] |
| I-266 | 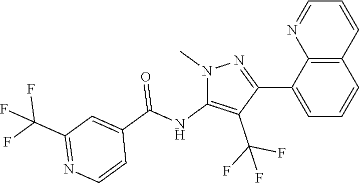 [SMILES: Cn1nc(-c2cccc3cccnc23)c(C(F)(F)F)c1NC(=O)c1ccnc(C(F)(F)F)c1] |
| I-267 | 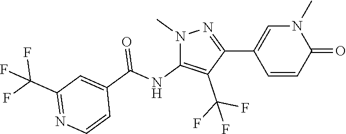 [SMILES: Cn1nc(-c2ccc(=O)n(C)c2)c(C(F)(F)F)c1NC(=O)c1ccnc(C(F)(F)F)c1] |
| I-268 | 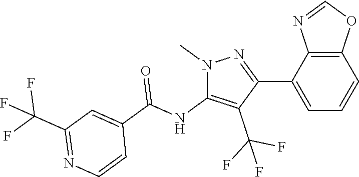 [SMILES: Cn1nc(-c2cccc3ocnc23)c(C(F)(F)F)c1NC(=O)c1ccnc(C(F)(F)F)c1] |
| I-269 | 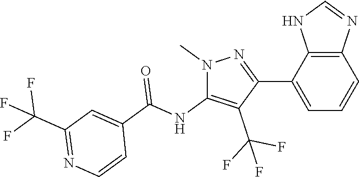 [SMILES: Cn1nc(-c2cccc3nc[nH]c23)c(C(F)(F)F)c1NC(=O)c1ccnc(C(F)(F)F)c1] |
| I-270 | 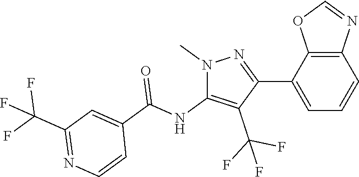 [SMILES: Cn1nc(-c2cccc3ncoc23)c(C(F)(F)F)c1NC(=O)c1ccnc(C(F)(F)F)c1] |
| I-271 | 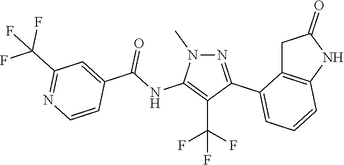 [SMILES: Cn1nc(-c2cccc3c2CC(=O)N3)c(C(F)(F)F)c1NC(=O)c1ccnc(C(F)(F)F)c1] |
| I-272 | 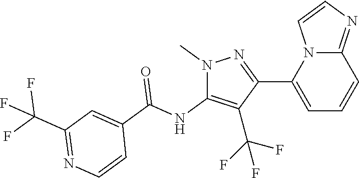 [SMILES: Cn1nc(-c2cccc3nccn23)c(C(F)(F)F)c1NC(=O)c1ccnc(C(F)(F)F)c1] |
| I-273 | 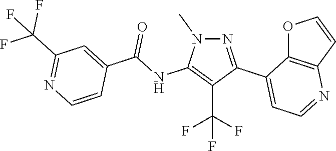 [SMILES: Cn1nc(-c2ccnc3ccoc23)c(C(F)(F)F)c1NC(=O)c1ccnc(C(F)(F)F)c1] |
| I-274 | 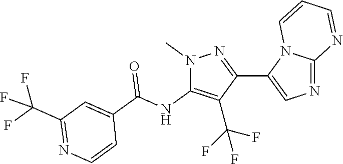 [SMILES: Cn1nc(-c2cnc3ncccn23)c(C(F)(F)F)c1NC(=O)c1ccnc(C(F)(F)F)c1] |
| I-275 | 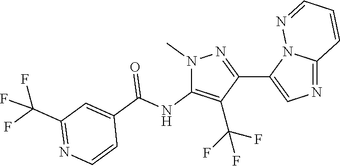 [SMILES: Cn1nc(-c2cnc3cccnn23)c(C(F)(F)F)c1NC(=O)c1ccnc(C(F)(F)F)c1] |
| I-276 | 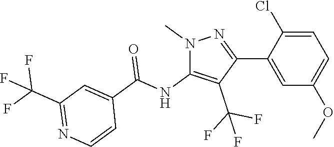 [SMILES: COc1ccc(Cl)c(-c2nn(C)c(NC(=O)c3ccnc(C(F)(F)F)c3)c2C(F)(F)F)c1] |
| I-277 | 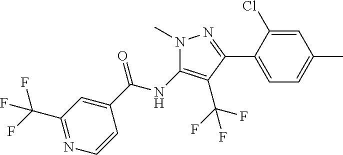 [SMILES: Cc1ccc(-c2nn(C)c(NC(=O)c3ccnc(C(F)(F)F)c3)c2C(F)(F)F)c(Cl)c1] |
| I-278 | 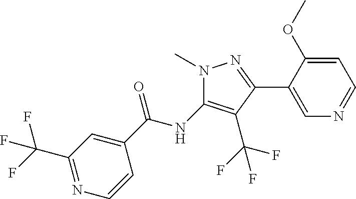 [SMILES: COc1ccncc1-c1nn(C)c(NC(=O)c2ccnc(C(F)(F)F)c2)c1C(F)(F)F] |
| I-279 | 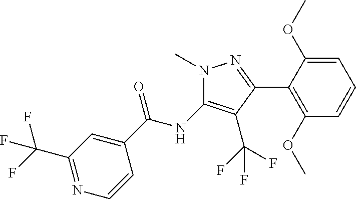 [SMILES: COc1cccc(OC)c1-c1nn(C)c(NC(=O)c2ccnc(C(F)(F)F)c2)c1C(F)(F)F] |
| I-280 | 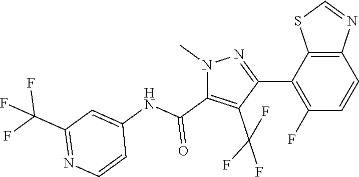 [SMILES: Cn1nc(-c2c(F)ccc3ncsc23)c(C(F)(F)F)c1C(=O)Nc1ccnc(C(F)(F)F)c1] |
| I-281 | 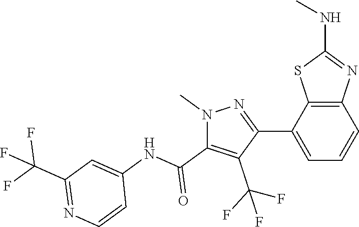 [SMILES: CNc1nc2cccc(-c3nn(C)c(C(=O)Nc4ccnc(C(F)(F)F)c4)c3C(F)(F)F)c2s1] |
| I-282 | 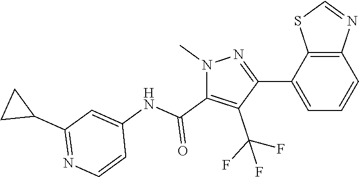 [SMILES: Cn1nc(-c2cccc3ncsc23)c(C(F)(F)F)c1C(=O)Nc1ccnc(C2CC2)c1] |
| I-283 | 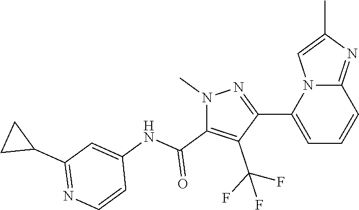 [SMILES: Cc1cn2c(-c3nn(C)c(C(=O)Nc4ccnc(C5CC5)c4)c3C(F)(F)F)cccc2n1] |
| I-284 | 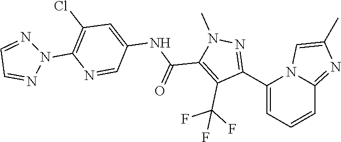 [SMILES: Cc1cn2c(-c3nn(C)c(C(=O)Nc4cnc(-n5nccn5)c(Cl)c4)c3C(F)(F)F)cccc2n1] |
| I-285 | 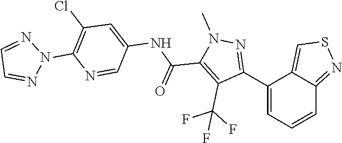 [SMILES: Cn1nc(-c2cccc3nscc23)c(C(F)(F)F)c1C(=O)Nc1cnc(-n2nccn2)c(Cl)c1] |
| I-286 | 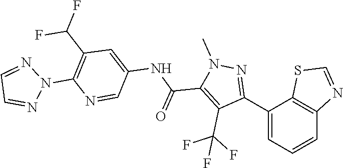 [SMILES: Cn1nc(-c2cccc3ncsc23)c(C(F)(F)F)c1C(=O)Nc1cnc(-n2nccn2)c(C(F)F)c1] |
| I-287 | 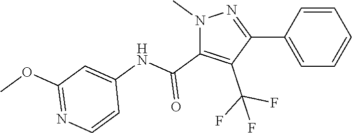 [SMILES: COc1cc(NC(=O)c2c(C(F)(F)F)c(-c3ccccc3)nn2C)ccn1] |
| I-288 | 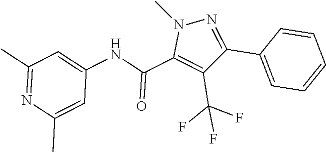 [SMILES: Cc1cc(NC(=O)c2c(C(F)(F)F)c(-c3ccccc3)nn2C)cc(C)n1] |
| I-289 | 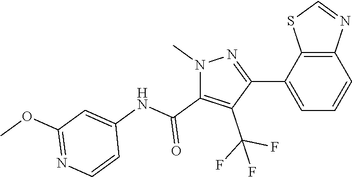 [SMILES: COc1cc(NC(=O)c2c(C(F)(F)F)c(-c3cccc4ncsc34)nn2C)ccn1] |
| I-290 | 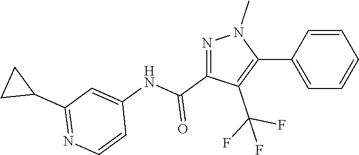 [SMILES: Cn1nc(C(=O)Nc2ccnc(C3CC3)c2)c(C(F)(F)F)c1-c1ccccc1] |
| I-291 | 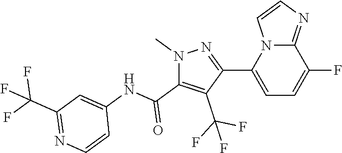 [SMILES: Cn1nc(-c2ccc(F)c3nccn23)c(C(F)(F)F)c1C(=O)Nc1ccnc(C(F)(F)F)c1] |
| I-292 | 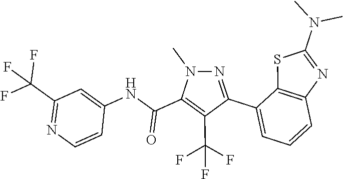 [SMILES: CN(C)c1nc2cccc(-c3nn(C)c(C(=O)Nc4ccnc(C(F)(F)F)c4)c3C(F)(F)F)c2s1] |
| I-293 | 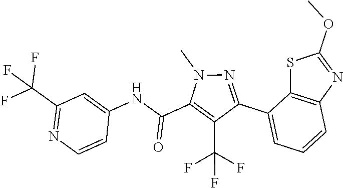 [SMILES: COc1nc2cccc(-c3nn(C)c(C(=O)Nc4ccnc(C(F)(F)F)c4)c3C(F)(F)F)c2s1] |
| I-294 | 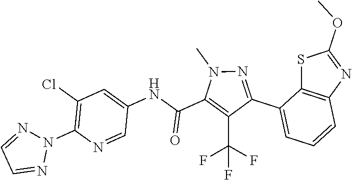 [SMILES: COc1nc2cccc(-c3nn(C)c(C(=O)Nc4cnc(-n5nccn5)c(Cl)c4)c3C(F)(F)F)c2s1] |
| I-295 | 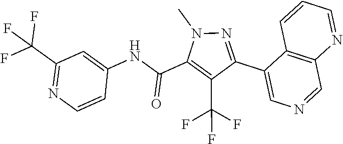 [SMILES: Cn1nc(-c2cncc3ncccc23)c(C(F)(F)F)c1C(=O)Nc1ccnc(C(F)(F)F)c1] |
| I-296 | 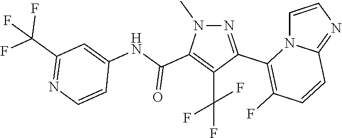 [SMILES: Cn1nc(-c2c(F)ccc3nccn23)c(C(F)(F)F)c1C(=O)Nc1ccnc(C(F)(F)F)c1] |
| I-297 | 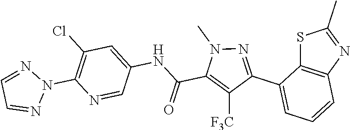 [SMILES: Cc1nc2cccc(-c3nn(C)c(C(=O)Nc4cnc(-n5nccn5)c(Cl)c4)c3C(F)(F)F)c2s1] |
| I-298 | 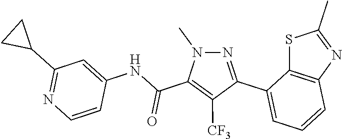 [SMILES: Cc1nc2cccc(-c3nn(C)c(C(=O)Nc4ccnc(C5CC5)c4)c3C(F)(F)F)c2s1] |
| I-299 | 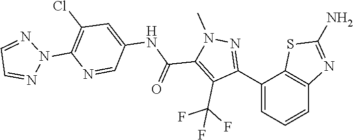 [SMILES: Cn1nc(-c2cccc3nc(N)sc23)c(C(F)(F)F)c1C(=O)Nc1cnc(-n2nccn2)c(Cl)c1] |

Another aspect of the invention provides a compound in Tables 1, 2, or 3, or a pharmaceutically acceptable salt thereof. In certain embodiments, the compound is a compound in Table 1, 2, or 3.

Methods for preparing compounds described herein are illustrated in the following synthetic scheme. The scheme is provided for the purpose of illustrating the invention, and is not intended to limit the scope or spirit of the invention. Starting materials shown in the scheme can be obtained from commercial sources or can be prepared based on procedures described in the literature.

In the schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in Protecting Groups in Organic Synthesis, 3^{rd} Edition, T. W. Greene and P. G. M. Wuts, John Wiley & Sons, 1999 and Greene's Protective Groups in Organic Synthesis, 5th Ed., (Peter G. M. Wuts, John Wiley & Sons: 2014), the entire contents of both of which are hereby incorporated by reference.

The synthetic route illustrated in Scheme 1 is a general method for preparing pyrazolylcarboxamides E. Reaction of pyrazolyl ester A with amine B under amide-bond formation conditions provides amide C. Reaction of amide C with dioxaborolane D under palladium coupling conditions provides pyrazolylcarboxamide E.

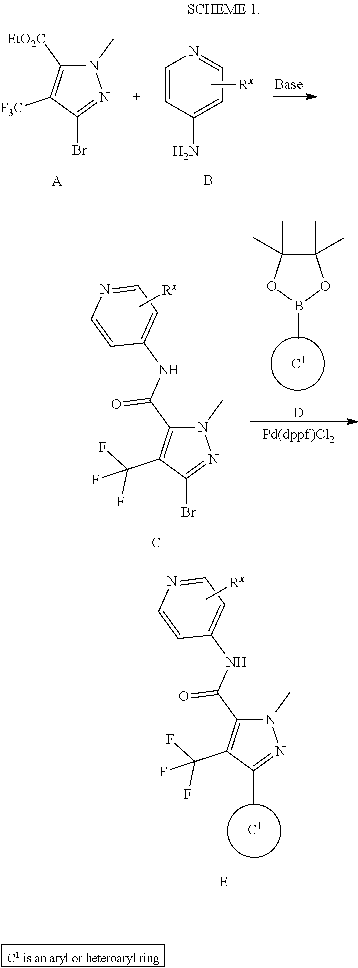

The modular synthetic route illustrated in Scheme 1 can be adjusted to provide additional pyrazolylcarboxamides and related compounds by conducting functional group transformations on the intermediate and final compounds. Such functional group transformations are well known in the art, as described in, for example, Comprehensive Organic Synthesis (B. M. Trost & I. Fleming, eds., 1991-1992); Organic Synthesis, 3^{rd} Ed. (Michael B. Smith, Wavefunction, Inc., Irvine: 2010); Modern Methods of Organic Synthesis. 4^{th} Ed. (William Carruthers and lain Coldham, Cambridge University Press, Cambridge: 2004); March's Advanced Organic Chemistry: Reactions, Mechanisms, and Structure, 8^{th} Ed., (Michael B. Smith, John Wiley & Sons, New York: 2020); and Comprehensive Organic Transformations: A Guide to Functional Group Preparations, 3^{rd} Ed. (Richard C. Larock, ed., John Wiley & Sons, New York: 2018).

### II. Therapeutic Applications of Pyrazolylcarboxamide Compounds

Compounds described herein are useful for treating a disease or condition mediated by MALT1. Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

Accordingly, one aspect of the invention provides a method of treating a disease or condition mediated by MALT1 in a subject. The method comprises administering to a subject in need thereof a therapeutically effective amount of a compound described herein, such as a compound of Formula I, I-1, II, or III, or other compounds in Section I, to treat the disease or condition. In certain embodiments, the compound is a compound of Formula Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or defined by one of the embodiments described above. Further description of exemplary diseases or conditions mediated by MALT1 is provided herein below.

Another aspect of the invention provides a method of inhibiting the activity of MALT1. The method comprises contacting a MALT1 with an effective amount of a compound described herein, such as a compound of Formula I, to inhibit the activity of said MALT1. In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above.

Another aspect of the invention provides for the use of a compound described herein (such as a compound of I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, II-1, Im-1, or In-1, or other compounds in Section I) in the manufacture of a medicament. In certain embodiments, the medicament is for treating a disease or condition described herein, such as an inflammatory disorder or an allergic disorder. In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above.

Another aspect of the invention provides for the use of a compound described herein (such as a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) for treating a disease or condition, such as a disease or condition described herein. In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above.

In certain embodiments, the subject is a human. In certain embodiments, the subject is an adult human. In certain embodiments, the subject is a pediatric human. In certain embodiments, the subject is a geriatric human.

### Exemplary Diseases or Conditions

Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

In certain embodiments, the disease or condition mediated by MALT1 is a proliferative disorder. In certain embodiments, the disease or condition mediated by MALT1 is an inflammatory disorder. In certain embodiments, the disease or condition mediated by MALT1 is an autoimmune disorder. In certain embodiments, the disease or condition mediated by MALT1 is a fibrotic disorder. In certain embodiments, the disease or condition mediated by MALT1 is a metabolic disorder. In certain embodiments, the disease or condition mediated by MALT1 is a cardiovascular disorder. In certain embodiments, the disease or condition mediated by MALT1 is a cerebrovascular disorder. In certain embodiments, the disease or condition mediated by MALT1 is a myeloid cell-driven hyper-inflammatory response in a COVID-19 infection.

In certain embodiments, the disease or condition mediated by MALT1 is cancer.

In certain embodiments, the cancer is selected from is non-small cell lung cancer (NSCLC), small cell lung cancer, colorectal cancer, rectal cancer, and pancreatic cancer. In certain embodiments, the cancer is selected from non-small cell lung cancer (NSCLC), pancreatic cancer, and colorectal cancer. In certain embodiments, the cancer is selected from non-small cell lung cancer (NSCLC) and pancreatic cancer.

In certain embodiments, the cancer is a solid tumor. In certain embodiments, the cancer is a melanoma, carcinoma, or blastoma. In certain embodiments, the cancer is a melanoma. In certain embodiments, the cancer is a carcinoma. In certain embodiments, the cancer is an adenocarcinoma. In certain embodiments, the cancer is a blastoma.

In certain embodiments, the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia. In certain embodiments, the cancer is lung cancer. In certain embodiments, the cancer is pancreatic cancer. In certain embodiments, the cancer is colorectal cancer. In certain embodiments, the cancer is breast cancer. In certain embodiments, the cancer is cervical cancer. In certain embodiments, the cancer is prostate cancer. In certain embodiments, the cancer is gastric cancer. In certain embodiments, the cancer is skin cancer. In certain embodiments, the cancer is liver cancer. In certain embodiments, the cancer is bile duct cancer. In certain embodiments, the cancer is nervous system cancer.

In certain embodiments, the cancer is breast adenocarcinoma, lung adenocarcinoma, pancreatic adenocarcinoma, cervical adenocarcinoma, colorectal adenocarcinoma, prostate adenocarcinoma, gastric adenocarcinoma, melanoma, lung squamous cell carcinoma, hepatocellular carcinoma, cholangiocarcinoma, glioblastoma, or neuroblastoma. In certain embodiments, the cancer is breast adenocarcinoma. In certain embodiments, the cancer is lung adenocarcinoma. In certain embodiments, the cancer is pancreatic adenocarcinoma. In certain embodiments, the cancer is cervical adenocarcinoma. In certain embodiments, the cancer is prostate adenocarcinoma. In certain embodiments, the cancer is gastric adenocarcinoma.

In certain embodiments, the cancer is melanoma.

In certain embodiments, the cancer is lung squamous cell carcinoma, hepatocellular carcinoma, or cholangiocarcinoma. In certain embodiments, the cancer is lung squamous cell carcinoma. In certain embodiments, the cancer is hepatocellular carcinoma. In certain embodiments, the cancer is cholangiocarcinoma.

In certain embodiments, the cancer is glioblastoma or neuroblastoma. In certain embodiments, the cancer is glioblastoma. In certain embodiments, the cancer is neuroblastoma.

In certain embodiments, the cancer is lung cancer, pancreatic cancer, or colorectal cancer. In certain embodiments, the cancer is non-small cell lung cancer, pancreatic cancer, or colorectal cancer. In certain embodiments, the cancer is lung cancer. In certain embodiments, the cancer is non-small cell lung cancer.

In certain embodiments, the cancer is a lymphoma or leukemia. In certain embodiments, the cancer is a B-cell lymphoma or chornic myelocytic leukemia.

In certain embodiments, the cancer is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, or a solid tumor such as a sarcoma or carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In certain embodiments, the cancer is MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

In certain embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In certain embodiments, the cancer is acoustic neuroma, astrocytoma (e.g. Grade I-Pilocytic Astrocytoma, Grade II-Low-grade Astrocytoma, Grade III-Anaplastic Astrocytoma, or Grade IV-Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In certain embodiments, the cancer is a type found more commonly in children than adults, such as brain stem glioma, craniopharyngioma, ependymoma, juvenile pilocytic astrocytoma (JPA), medulloblastoma, optic nerve glioma, pincal tumor, primitive neuroectodermal tumors (PNET), or rhabdoid tumor.

In certain embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, fallopian tube cancer, papillary serous cystadenocarcinoma, uterine papillary serous carcinoma (UPSC), prostate cancer, testicular cancer, gallbladder cancer, hepatocholangiocarcinoma, soft tissue and bone synovial sarcoma, rhabdomyosarcoma, osteosarcoma, chondrosarcoma, Ewing sarcoma, anaplastic thyroid cancer, adrenocortical adenoma, pancreatic cancer, pancreatic ductal carcinoma, pancreatic adenocarcinoma, gastrointestinal/stomach (GIST) cancer, lymphoma, squamous cell carcinoma of the head and neck (SCCHN), salivary gland cancer, glioma, or brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; and medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In certain embodiments, the cancer is hepatoblastoma. In certain embodiments, the cancer is colon cancer. In certain embodiments, the cancer is rectal cancer. In certain embodiments, the cancer is ovarian cancer, or ovarian carcinoma. In certain embodiments, the cancer is ovarian epithelial cancer. In certain embodiments, the cancer is fallopian tube cancer. In certain embodiments, the cancer is papillary serous cystadenocarcinoma. In certain embodiments, the cancer is uterine papillary serous carcinoma (UPSC). In certain embodiments, the cancer is hepatocholangiocarcinoma. In certain embodiments, the cancer is soft tissue and bone synovial sarcoma. In certain embodiments, the cancer is rhabdomyosarcoma. In certain embodiments, the cancer is osteosarcoma. In certain embodiments, the cancer is anaplastic thyroid cancer. In certain embodiments, the cancer is adrenocortical carcinoma. In certain embodiments, the cancer is pancreatic cancer, or pancreatic ductal carcinoma. In certain embodiments, the cancer is pancreatic adenocarcinoma. In certain embodiments, the cancer is glioma. In certain embodiments, the cancer is malignant peripheral nerve sheath tumors (MPNST). In certain embodiments, the cancer is neurofibromatosis-1 associated MPNST. In certain embodiments, the cancer is Waldenstrom's macroglobulinemia. In certain embodiments, the cancer is medulloblastoma.

In certain embodiments, the cancer is a lymphoma. In certain embodiments, the cancer is a leukemia. In certain embodiments, the cancer is Hodgkin's lymphoma. In certain embodiments, the cancer is non-Hodgkin's lymphoma. In certain embodiments, the cancer is Burkitt's lymphoma. In certain embodiments, the cancer is diffuse large B-cell lymphoma (DLBCL). In certain embodiments, the cancer is MALT lymphoma. In certain embodiments, the cancer is germinal center B-cell-like diffuse large B-cell lymphoma (GCB-DLBCL) or primary mediastinal B-cell lymphoma (PMBL). In certain embodiments, the cancer is activated B-cell-like diffuse large B-cell lymphoma (ABC-DLBCL). In certain embodiments, the cancer is a hematological cancer.

In certain embodiments, the proliferative disease is a cancer associated with or dependent on a MALT1 fusion protein (e.g., API2-MALT1). In certain embodiments, the proliferative disease is a cancer associated with dependence on B-cell lymphoma 10 (Bcl10). In certain embodiments, the proliferative disease is a cancer associated with dependence on caspase recruitment domain-containing protein (CARD1). In certain embodiments, the proliferative disease is a cancer associated with dependence on NF-κB. In certain embodiments, the cancer is a hematological malignancy.

Additional exemplary cancers include but are not limited to acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; parancoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); Burkitt lymphoma; primary intraocular lymphoma: classic Hodgkin lymphoma; biphenotypic acute leukemia; T cell lymphoma; nasal-type T cell lymphoma; enteropathy-type T-cell lymphoma; subcutaneous panniculitis-like T-cell lymphoma; blastic NK-cell lymphoma; T-cell prolymphocytic leukemia, and NK-cell leukemia.

In certain embodiments, the cancer is a hematological malignancy. Exemplary hematological malignancies include but are not limited to leukemia, such as acute lymphoblastic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), acute non-lymphocytic leukemia (ANLL), acute promyelocytic leukemia (APL), and acute myelomonocytic leukemia (AMMoL); lymphoma, such as Hodgkin lymphoma (HL) (e.g., B-cell HL, T-cell HL) and non-Hodgkin lymphoma (NHL) (e.g., B-cell NHI, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, Waldenstrom's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; multiple myeloma (MM); heavy chain disease (e.g., alpha chain disease, gamma chain disease, mu chain disease), polycythemia vera, Wilm's tumor, and Ewing's sarcoma.

In certain embodiments, said disease or condition mediated by MALT1 is a multiple myeloma. In certain embodiments, said disease or condition mediated by MALT1 is a leukemia (e.g., acute lymphocytic leukemia, acute and chronic myelogenous leukemia, chronic lymphocytic leukemia, acute lymphoblastic leukemia, chronic myelomonocytic leukemia, or promyelocytic leukemia).

In certain embodiments, said disease or condition mediated by MALT1 is a lymphoma (e.g., B-cell lymphoma, T-cell lymphoma, mantle cell lymphoma, diffuse large B cell lymphoma, hairy cell lymphoma, Burkitt's lymphoma, mast cell tumors, Hodgkin's disease or non-Hodgkin's disease). In certain embodiments, said disease or condition mediated by MALT1 is myelodysplastic syndrome. In certain embodiments, said disease or condition mediated by MALT1 is fibrosarcoma. In certain embodiments, said disease or condition mediated by MALT1 is rhabdomyosarcoma. In certain embodiments, said disease or condition mediated by MALT1 is astrocytoma. In certain embodiments, said disease or condition mediated by MALT1 is neuroblastoma. In certain embodiments, said disease or condition mediated by MALT1 is glioma and schwannomas. In certain embodiments, said disease or condition mediated by MALT1 is melanoma. In certain embodiments, said disease or condition mediated by MALT1 is seminoma. In certain embodiments, said disease or condition mediated by MALT1 is teratocarcinoma. In certain embodiments, said disease or condition mediated by MALT1 is osteosarcoma. In certain embodiments, said disease or condition mediated by MALT1 is xenoderoma pigmentosum. In certain embodiments, said disease or condition mediated by MALT1 is keratoctanthoma. In certain embodiments, said disease or condition mediated by MALT1 is thyroid follicular cancer. In certain embodiments, said disease or condition mediated by MALT1 is Kaposi's sarcoma. In certain embodiments, said disease or condition mediated by MALT1 is melanoma. In certain embodiments, said disease or condition mediated by MALT1 is teratoma. In certain embodiments, said disease or condition mediated by MALT1 is rhabdomyosarcoma. In certain embodiments, said disease or condition mediated by MALT1 is a metastatic and bone disorder. In certain embodiments, said disease or condition mediated by MALT1 is cancer of the bone. In certain embodiments, said disease or condition mediated by MALT1 is cancer of the mouth/pharynx. In certain embodiments, said disease or condition mediated by MALT1 is cancer of the esophagus. In certain embodiments, said disease or condition mediated by MALT1 is cancer of the larynx. In certain embodiments, said disease or condition mediated by MALT1 is cancer of the stomach. In certain embodiments, said disease or condition mediated by MALT1 is cancer of the intestine. In certain embodiments, said disease or condition mediated by MALT1 is cancer of the colon. In certain embodiments, said disease or condition mediated by MALT1 is cancer of the rectum. In certain embodiments, said disease or condition mediated by MALT1 is cancer of the lung (e.g., non-small cell lung cancer or small cell lung cancer). In certain embodiments, said disease or condition mediated by MALT1 is cancer of the liver. In certain embodiments, said disease or condition mediated by MALT1 is cancer of the pancreas. In certain embodiments, said disease or condition mediated by MALT1 is cancer of the nerve. In certain embodiments, said disease or condition mediated by MALT1 is cancer of the brain (e.g., glioma or glioblastoma multiforme). In certain embodiments, said disease or condition mediated by MALT1 is cancer of the head and neck. In certain embodiments, said disease or condition mediated by MALT1 is cancer of the throat. In certain embodiments, said disease or condition mediated by MALT1 is cancer of the ovary. In certain embodiments, said disease or condition mediated by MALT1 is cancer of the uterus. In certain embodiments, said disease or condition mediated by MALT1 is cancer of the prostate. In certain embodiments, said disease or condition mediated by MALT1 is cancer of the testis. In certain embodiments, said disease or condition mediated by MALT1 is cancer of the bladder. In certain embodiments, said disease or condition mediated by MALT1 is cancer of the kidney. In certain embodiments, said disease or condition mediated by MALT1 is cancer of the breast. In certain embodiments, said disease or condition mediated by MALT1 is cancer of the gall bladder. In certain embodiments, said disease or condition mediated by MALT1 is cancer of the cervix. In certain embodiments, said disease or condition mediated by MALT1 is cancer of the thyroid. In certain embodiments, said disease or condition mediated by MALT1 is cancer of the prostate. In certain embodiments, said disease or condition mediated by MALT1 is cancer of the skin (e.g., skin squamous cell carcinoma). In certain embodiments, said disease or condition mediated by MALT1 is a solid tumor. In certain embodiments, said disease or condition mediated by MALT1 is gastric cancer. In certain embodiments, said disease or condition mediated by MALT1 is hepatocellular carcinoma. In certain embodiments, said disease or condition mediated by MALT1 is a peripheral nerve sheath tumor. In certain embodiments, said disease or condition mediated by MALT1 is pulmonary arterial hypertension.

In certain embodiments, the disease is a cancer associated with a viral infection. In certain embodiments, the disease is a cancer resulting from infection with an oncovirus. In certain embodiments, the oncovirus is hepatitis A, hepatitis B, hepatitis C, human T-lymphotropic virus (HTLV), human papillomavirus (HPV), Kaposi's sarcoma-associated herpesvirus (HHV-8), Merkel cell polyomavirus, or Epstein-Barr virus (EBV). In certain embodiments, the disease is human T-lymphotropic virus. In certain embodiments, the disease is Kaposi's sarcoma-associated herpesvirus. In certain embodiments, the disease is Epstein-Barr virus. Leukemias and lymphomas which may be associated with an oncoviral include: for HTLV, adult T-cell leukemia; for HHV-8, Castleman's disease and primary effusion lymphoma; and for EBV, Burkitt's lymphoma, Hogdkin's lymphoma, and post-transplant lymphoproliferative disease.

In certain embodiments, said disease or condition mediated by MALT1 is an inflammatory disorder or allergic disorder. In certain embodiments, said disease or condition mediated by MALT1 is an inflammatory disorder, such as autoimmune disorders, chronic inflammatory disorders, acute inflammatory disorders, auto-inflammatory disorders, fibrotic disorders, metabolic disorders, neoplasias, cardiovascular or cerebrovascular disorders, and myeloid cell-driven hyper-inflammatory response in COVID-19 infections. In certain embodiments, said disease or condition mediated by MALT1 is an allergic disorder, such as asthma and allergic rhinitis.

In certain embodiments, said disease or condition mediated by MALT1 is a disease or disorder of tissues and systemic disease [e.g., systemic lupus erythematosus (SLE); immune thrombocytopenia purpura (ITP); autoimmune hemolytic anemia (AHA); autoimmune neutropenia (AlN); Evans syndrome; proliferative and hyperproliferative diseases, such as cancer, atherosclerosis, rheumatoid arthritis, psoriasis, idiopathic pulmonary fibrosis, scleroderma, cirrhosis of the liver; and Acquired Immunodeficiency Syndrome (AIDS)]. In certain embodiments, said disease or condition mediated by MALT1 is an immunologically-mediated disease, such as allograft rejection (e.g., rejection of transplanted organs or tissues). In certain embodiments, said disease or condition mediated by MALT1 is a tissue injury (e.g., associated with organ transplant or revascularization procedures). In certain embodiments, said disease or condition mediated by MALT1 is a disease or disorder of the respiratory tract (e.g., asthma). In certain embodiments, said disease or condition mediated by MALT1 is allergic rhinitis. In certain embodiments, said disease or condition mediated by MALT1 is a disease or disorder of the bone and joints (e.g., arthritis, rheumatoid arthritis). In certain embodiments, said disease or condition mediated by MALT1 is a disease or disorder of the skin. In certain embodiments, said disease or condition mediated by MALT1 is a disease or disorder of the gastrointestinal tract.

In certain embodiments, said disease or condition mediated by MALT1 is a reversible obstructive airways disease, such as asthma (e.g., bronchial asthma, allergic asthma, intrinsic asthma, extrinsic asthma, and dust asthma). In certain embodiments, said disease or condition mediated by MALT1 is chronic or inveterate asthma (e.g., late asthma airways hyper-responsiveness). In certain embodiments, said disease or condition mediated by MALT1 is bronchitis. In certain embodiments, said disease or condition mediated by MALT1 is a condition characterized by an inflammation of the nasal mucus membrane. In certain embodiments, said disease or condition mediated by MALT1 is acute rhinitis. In certain embodiments, said disease or condition mediated by MALT1 is allergic rhinitis. In certain embodiments, said disease or condition mediated by MALT1 is atrophic rhinitis. In certain embodiments, said disease or condition mediated by MALT1 is chronic rhinitis (e.g., rhinitis caseosa, hypertrophic rhinitis, rhinitis purulenta, rhinitis sicca, and rhinitis medicamentosa). In certain embodiments, said disease or condition mediated by MALT1 is membranous rhinitis (e.g., croupous rhinitis, fibrinous rhinitis, pseudomembranous rhinitis, and scrofoulous rhinitis). In certain embodiments, said disease or condition mediated by MALT1 is seasonal rhinitis [e.g., rhinitis nervosa (hay fever), vasomotor rhinitis, sarcoidosis, farmer's lung, and related diseases, such as fibroid lung and idiopathic interstitial pneumonia].

In certain embodiments, said disease or condition mediated by MALT1 includes pannus formation. In certain embodiments, said disease or condition mediated by MALT1 does not include pannus formation. In certain embodiments, said disease or condition mediated by MALT1 is rheumatoid arthritis. In certain embodiments, said disease or condition mediated by MALT1 is seronegative spondyloarthropathis (e.g., ankylosing spondylitis, psoriatic arthritis, and Reiter's disease). In certain embodiments, said disease or condition mediated by MALT1 is Behcet's disease. In certain embodiments, said disease or condition mediated by MALT1 is Sjogren's syndrome. In certain embodiments, said disease or condition mediated by MALT1 is systemic sclerosis.

In certain embodiments, said disease or condition mediated by MALT1 is psoriasis. In certain embodiments, said disease or condition mediated by MALT1 is systemic sclerosis. In certain embodiments, said disease or condition mediated by MALT1 is atopical dermatitis. In certain embodiments, said disease or condition mediated by MALT1 is contact dermatitis. In certain embodiments, said disease or condition mediated by MALT1 is eczematous dermatitis. In certain embodiments, said disease or condition mediated by MALT1 is seborrhoetic dermatitis. In certain embodiments, said disease or condition mediated by MALT1 is Lichen planus. In certain embodiments, said disease or condition mediated by MALT1 is Pemphigus. In certain embodiments, said disease or condition mediated by MALT1 is bullous Pemphigus. In certain embodiments, said disease or condition mediated by MALT1 is epidermolysis bullosa. In certain embodiments, said disease or condition mediated by MALT1 is urticaria. In certain embodiments, said disease or condition mediated by MALT1 is angiodermas. In certain embodiments, said disease or condition mediated by MALT1 is vasculitides. In certain embodiments, said disease or condition mediated by MALT1 is erythemas. In certain embodiments, said disease or condition mediated by MALT1 is cutaneous eosinophilias. In certain embodiments, said disease or condition mediated by MALT1 is uveitis. In certain embodiments, said disease or condition mediated by MALT1 is Alopecia. In certain embodiments, said disease or condition mediated by MALT1 is areata. In certain embodiments, said disease or condition mediated by MALT1 is vernal conjunctivitis.

In certain embodiments, said disease or condition mediated by MALT1 is Coeliac disease. In certain embodiments, said disease or condition mediated by MALT1 is proctitis. In certain embodiments, said disease or condition mediated by MALT1 is eosinophilic gastroenteritis. In certain embodiments, said disease or condition mediated by MALT1 is mastocytosis. In certain embodiments, said disease or condition mediated by MALT1 is pancreatitis. In certain embodiments, said disease or condition mediated by MALT1 is Crohn's disease. In certain embodiments, said disease or condition mediated by MALT1 is ulcerative colitis. In certain embodiments, said disease or condition mediated by MALT1 is a food-related allergy having effects remote from the gut (e.g., migraine, rhinitis, and eczema).

In certain embodiments, said disease or condition mediated by MALT1 is multiple sclerosis. In certain embodiments, said disease or condition mediated by MALT1 is artherosclerosis. In certain embodiments, said disease or condition mediated by MALT1 is acquired immunodeficiency syndrome (AIDS). In certain embodiments, said disease or condition mediated by MALT1 is lupus. In certain embodiments, said disease or condition mediated by MALT1 is lupus erythematosus. In certain embodiments, said disease or condition mediated by MALT1 is systemic lupus erythematosus. In certain embodiments, said disease or condition mediated by MALT1 is Hashimoto's thyroiditis. In certain embodiments, said disease or condition mediated by MALT1 is myasthenia gravis. In certain embodiments, said disease or condition mediated by MALT1 is type I diabetes. In certain embodiments, said disease or condition mediated by MALT1 is nephrotic syndrome. In certain embodiments, said disease or condition mediated by MALT1 is eosinophilia fasciitis. In certain embodiments, said disease or condition mediated by MALT1 is hyper IgE syndrome. In certain embodiments, said disease or condition mediated by MALT1 is lepromatous leprosy. In certain embodiments, said disease or condition mediated by MALT1 is sezary syndrome. In certain embodiments, said disease or condition mediated by MALT1 is idiopathic thrombocytopenia purpura. In certain embodiments, said disease or condition mediated by MALT1 is restenosis following angioplasty. In certain embodiments, said disease or condition mediated by MALT1 is a tumor (e.g., leukemia, lymphomas). In certain embodiments, said disease or condition mediated by MALT1 is artherosclerosis.

In certain embodiments, said disease or condition mediated by MALT1 is acute chronic allograft rejection (e.g., following transplantation of kidney, heart, liver, lung, bone marrow, skin, or cornea). In certain embodiments, said disease or condition mediated by MALT1 is chronic allograft rejection (e.g., following transplantation of kidney, heart, liver, lung, bone marrow, skin, or cornea). In certain embodiments, said disease or condition mediated by MALT1 is chronic graft-versus-host disease.

In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a fibrotic disorder. In certain embodiments, said disease or condition mediated by MALT1 is a metabolic disorder. In certain embodiments, said disease or condition mediated by MALT1 is a neoplasia. In certain embodiments, said disease or condition mediated by MALT1 is a cardiovascular or cerebrovascular disorder. In certain embodiments, said disease or condition mediated by MALT1 is a myeloid cell-driven hyper-inflammatory response in COVID-19 infections.

In certain embodiments, said disease or condition mediated by MALT1 is an autoimmune disorder. In certain embodiments, said disease or condition mediated by MALT1 is a chronic inflammatory disorder. In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a combination of one, two, or all three of a chronic inflammatory disorder, an acute inflammatory disorder, and an auto-inflammatory disorder.

In certain embodiments, said disease or condition mediated by MALT1 is an inflammatory bowel disease (e.g., ulcerative colitis or Crohn's disease). In certain embodiments, said disease or condition mediated by MALT1 is multiple sclerosis. In certain embodiments, said disease or condition mediated by MALT1 is psoriasis. In certain embodiments, said disease or condition mediated by MALT1 is arthritis. In certain embodiments, said disease or condition mediated by MALT1 is rheumatoid arthritis. In certain embodiments, said disease or condition mediated by MALT1 is osteoarthritis. In certain embodiments, said disease or condition mediated by MALT1 is juvenile arthritis. In certain embodiments, said disease or condition mediated by MALT1 is psoriatic arthritis. In certain embodiments, said disease or condition mediated by MALT1 is reactive arthritis. In certain embodiments, said disease or condition mediated by MALT1 is ankylosing spondylitis. In certain embodiments, said disease or condition mediated by MALT1 is cryopyrin-associated periodic syndromes. In certain embodiments, said disease or condition mediated by MALT1 is Muckle-Wells syndrome. In certain embodiments, said disease or condition mediated by MALT1 is familial cold auto-inflammatory syndrome. In certain embodiments, said disease or condition mediated by MALT1 is neonatal-onset multisystem inflammatory disease. In certain embodiments, said disease or condition mediated by MALT1 is TNF receptor-associated periodic syndrome. In certain embodiments, said disease or condition mediated by MALT1 is acute and chronic pancreatitis. In certain embodiments, said disease or condition mediated by MALT1 is atherosclerosis. In certain embodiments, said disease or condition mediated by MALT1 is gout. In certain embodiments, said disease or condition mediated by MALT1 is a fibrotic disorder (e.g., hepatic fibrosis or idiopathic pulmonary fibrosis). In certain embodiments, said disease or condition mediated by MALT1 is nephropathy. In certain embodiments, said disease or condition mediated by MALT1 is sarcoidosis. In certain embodiments, said disease or condition mediated by MALT1 is scleroderma. In certain embodiments, said disease or condition mediated by MALT1 is anaphylaxis. In certain embodiments, said disease or condition mediated by MALT1 is diabetes (e.g., diabetes mellitus type 1 or diabetes mellitus type 2). In certain embodiments, said disease or condition mediated by MALT1 is diabetic retinopathy. In certain embodiments, said disease or condition mediated by MALT1 is Still's disease. In certain embodiments, said disease or condition mediated by MALT1 is vasculitis. In certain embodiments, said disease or condition mediated by MALT1 is sarcoidosis. In certain embodiments, said disease or condition mediated by MALT1 is pulmonary inflammation. In certain embodiments, said disease or condition mediated by MALT1 is respiratory failure. In certain embodiments, said disease or condition mediated by MALT1 is acute respiratory distress syndrome. In certain embodiments, said disease or condition mediated by MALT1 is chronic eosinophilic pneumonia. In certain embodiments, said disease or condition mediated by MALT1 is wet and dry age-related macular degeneration. In certain embodiments, said disease or condition mediated by MALT1 is autoimmune hemolytic syndromes. In certain embodiments, said disease or condition mediated by MALT1 is autoimmune and inflammatory hepatitis. In certain embodiments, said disease or condition mediated by MALT1 is autoimmune neuropathy. In certain embodiments, said disease or condition mediated by MALT1 is autoimmune ovarian failure. In certain embodiments, said disease or condition mediated by MALT1 is autoimmune orchitis. In certain embodiments, said disease or condition mediated by MALT1 is autoimmune thrombocytopenia. In certain embodiments, said disease or condition mediated by MALT1 is silicone implant-associated autoimmune disease. In certain embodiments, said disease or condition mediated by MALT1 is Sjogren's syndrome. In certain embodiments, said disease or condition mediated by MALT1 is familial Mediterranean fever. In certain embodiments, said disease or condition mediated by MALT1 is systemic lupus erythematosus. In certain embodiments, said disease or condition mediated by MALT1 is vasculitis syndromes (e.g., temporal. Takayasu's and giant cell arteritis, Behcet's disease or Wegener's granulomatosis). In certain embodiments, said disease or condition mediated by MALT1 is vitiligo. In certain embodiments, said disease or condition mediated by MALT1 is secondary hematologic manifestation of autoimmune diseases (e.g., anemias). In certain embodiments, said disease or condition mediated by MALT1 is drug-induced autoimmunity. In certain embodiments, said disease or condition mediated by MALT1 is Hashimoto's thyroiditis. In certain embodiments, said disease or condition mediated by MALT1 is hypophysitis. In certain embodiments, said disease or condition mediated by MALT1 is idiopathic thrombocytic pupura. In certain embodiments, said disease or condition mediated by MALT1 is metal-induced autoimmunity. In certain embodiments, said disease or condition mediated by MALT1 is myasthenia gravis. In certain embodiments, said disease or condition mediated by MALT1 is pemphigus. In certain embodiments, said disease or condition mediated by MALT1 is autoimmune deafness (e.g., Meniere's disease). In certain embodiments, said disease or condition mediated by MALT1 is Goodpasture's syndrome. In certain embodiments, said disease or condition mediated by MALT1 is Graves' disease. In certain embodiments, said disease or condition mediated by MALT1 is an HW-related autoimmune syndromes. In certain embodiments, said disease or condition mediated by MALT1 is Gullain-Barre disease. In certain embodiments, said disease or condition mediated by MALT1 is Addison's disease. In certain embodiments, said disease or condition mediated by MALT1 is anti-phospholipid syndrome. In certain embodiments, said disease or condition mediated by MALT1 is asthma. In certain embodiments, said disease or condition mediated by MALT1 is atopic dermatitis. In certain embodiments, said disease or condition mediated by MALT1 is Celiac disease. In certain embodiments, said disease or condition mediated by MALT1 is Cushing's syndrome. In certain embodiments, said disease or condition mediated by MALT1 is dermatomyositis. In certain embodiments, said disease or condition mediated by MALT1 is idiopathic adrenal atrophy. In certain embodiments, said disease or condition mediated by MALT1 is idiopathic thrombocytopenia. In certain embodiments, said disease or condition mediated by MALT1 is Kawasaki syndrome. In certain embodiments, said disease or condition mediated by MALT1 is Lambert-Eaton Syndrome. In certain embodiments, said disease or condition mediated by MALT1 is pernicious anemia. In certain embodiments, said disease or condition mediated by MALT1 is pollinosis. In certain embodiments, said disease or condition mediated by MALT1 is polyarteritis nodosa. In certain embodiments, said disease or condition mediated by MALT1 is primary biliary cirrhosis. In certain embodiments, said disease or condition mediated by MALT1 is primary sclerosing cholangitis. In certain embodiments, said disease or condition mediated by MALT1 is Raynaud's disease. In certain embodiments, said disease or condition mediated by MALT1 is Raynaud's phenomenon. In certain embodiments, said disease or condition mediated by MALT1 is Reiter's Syndrome. In certain embodiments, said disease or condition mediated by MALT1 is relapsing polychondritis. In certain embodiments, said disease or condition mediated by MALT1 is Schmidt's syndrome. In certain embodiments, said disease or condition mediated by MALT1 is thyrotoxidosis. In certain embodiments, said disease or condition mediated by MALT1 is sepsis. In certain embodiments, said disease or condition mediated by MALT1 is septic shock. In certain embodiments, said disease or condition mediated by MALT1 is endotoxic shock. In certain embodiments, said disease or condition mediated by MALT1 is exotoxin-induced toxic shock. In certain embodiments, said disease or condition mediated by MALT1 is gram negative sepsis. In certain embodiments, said disease or condition mediated by MALT1 is toxic shock syndrome. In certain embodiments, said disease or condition mediated by MALT1 is glomerulonephritis. In certain embodiments, said disease or condition mediated by MALT1 is peritonitis. In certain embodiments, said disease or condition mediated by MALT1 is interstitial cystitis. In certain embodiments, said disease or condition mediated by MALT1 is hyperoxia-induced inflammations. In certain embodiments, said disease or condition mediated by MALT1 is chronic obstructive pulmonary disease (COPD). In certain embodiments, said disease or condition mediated by MALT1 is emphysema. In certain embodiments, said disease or condition mediated by MALT1 is nasal inflammation. In certain embodiments, said disease or condition mediated by MALT1 is vasculitis. In certain embodiments, said disease or condition mediated by MALT1 is graft vs. host reaction (e.g., graft vs. host disease). In certain embodiments, said disease or condition mediated by MALT1 is allograft rejections (e.g., acute allograft rejection or chronic allograft rejection). In certain embodiments, said disease or condition mediated by MALT1 is early transplantation rejection (e.g., acute allograft rejection). In certain embodiments, said disease or condition mediated by MALT1 is reperfusion injury. In certain embodiments, said disease or condition mediated by MALT1 is pain (e.g., acute pain, chronic pain, neuropathic pain, or fibromyalgia). In certain embodiments, said disease or condition mediated by MALT1 is a chronic infection. In certain embodiments, said disease or condition mediated by MALT1 is meningitis. In certain embodiments, said disease or condition mediated by MALT1 is encephalitis. In certain embodiments, said disease or condition mediated by MALT1 is myocarditis. In certain embodiments, said disease or condition mediated by MALT1 is gingivitis. In certain embodiments, said disease or condition mediated by MALT1 is post-surgical trauma. In certain embodiments, said disease or condition mediated by MALT1 is tissue injury. In certain embodiments, said disease or condition mediated by MALT1 is traumatic brain injury. In certain embodiments, said disease or condition mediated by MALT1 is enterocolitis. In certain embodiments, said disease or condition mediated by MALT1 is sinusitis. In certain embodiments, said disease or condition mediated by MALT1 is uveitis. In certain embodiments, said disease or condition mediated by MALT1 is ocular inflammation. In certain embodiments, said disease or condition mediated by MALT1 is optic neuritis. In certain embodiments, said disease or condition mediated by MALT1 is gastric ulcers. In certain embodiments, said disease or condition mediated by MALT1 is esophagitis. In certain embodiments, said disease or condition mediated by MALT1 is peritonitis. In certain embodiments, said disease or condition mediated by MALT1 is periodontitis. In certain embodiments, said disease or condition mediated by MALT1 is dermatomyositis. In certain embodiments, said disease or condition mediated by MALT1 is gastritis. In certain embodiments, said disease or condition mediated by MALT1 is myositis. In certain embodiments, said disease or condition mediated by MALT1 is polymyalgia. In certain embodiments, said disease or condition mediated by MALT1 is pneumonia. In certain embodiments, said disease or condition mediated by MALT1 is bronchitis. In certain embodiments, the disease or condition mediated by MALT1 is endometriosis. In certain embodiments, the disease or condition mediated by MALT1 is necrotizing vasculitis. In certain embodiments, the disease or condition mediated by MALT1 is lymphadenitis. In certain embodiments, the disease or condition mediated by MALT1 is periarteritis nodosa. In certain embodiments, the disease or condition mediated by MALT1 is anti-phospholipid antibody syndrome. In certain embodiments, the disease or condition mediated by MALT1 is pemphigus vulgaris. In certain embodiments, the disease or condition mediated by MALT1 is Lyme disease. In certain embodiments, the disease or condition mediated by MALT1 is cardiomyopathy. In certain embodiments, the disease or condition mediated by MALT1 isrheumatic fever. In certain embodiments, the disease or condition mediated by MALT1 is a blistering disorder. In certain embodiments, the disease or condition mediated by MALT1 is an antibody-mediated vasculitis syndrome. In certain embodiments, the disease or condition mediated by MALT1 is an immune-complex vasculitide. In certain embodiments, the disease or condition mediated by MALT1 is oedema. In certain embodiments, the disease or condition mediated by MALT1 is embolism. In certain embodiments, the disease or condition mediated by MALT1 is fibrosis. In certain embodiments, the disease or condition mediated by MALT1 is silicosis. In certain embodiments, the disease or condition mediated by MALT1 is BENTA disease. In certain embodiments, the disease or condition mediated by MALT1 is berylliosis.

In certain embodiments, said disease or condition mediated by MALT1 is systemic sclerosis/scleroderma. In certain embodiments, said disease or condition mediated by MALT1 is lupus nephritis. In certain embodiments, said disease or condition mediated by MALT1 is connective tissue disease. In certain embodiments, said disease or condition mediated by MALT1 is wound healing. In certain embodiments, said disease or condition mediated by MALT1 is surgical scarring. In certain embodiments, said disease or condition mediated by MALT1 is spinal cord injury. In certain embodiments, said disease or condition mediated by MALT1 is CNS scarring. In certain embodiments, said disease or condition mediated by MALT1 is acute lung injury. In certain embodiments, said disease or condition mediated by MALT1 is pulmonary fibrosis (e.g., idiopathic pulmonary fibrosis or cystic fibrosis). In certain embodiments, said disease or condition mediated by MALT1 is chronic obstructive pulmonary disease. In certain embodiments, said disease or condition mediated by MALT1 is adult respiratory distress syndrome. In certain embodiments, said disease or condition mediated by MALT1 is acute lung injury. In certain embodiments, said disease or condition mediated by MALT1 is drug-induced lung injury. In certain embodiments, said disease or condition mediated by MALT1 is glomerulonephritis. In certain embodiments, said disease or condition mediated by MALT1 is chronic kidney disease (e.g., diabetic nephropathy). In certain embodiments, said disease or condition mediated by MALT1 is hypertension-induced nephropathy. In certain embodiments, said disease or condition mediated by MALT1 is alimentary track or gastrointestinal fibrosis. In certain embodiments, said disease or condition mediated by MALT1 is renal fibrosis. In certain embodiments, said disease or condition mediated by MALT1 is hepatic or biliary fibrosis. In certain embodiments, said disease or condition mediated by MALT1 is liver fibrosis (e.g., nonalcoholic steatohepatitis, hepatitis C, or hepatocellular carcinoma). In certain embodiments, said disease or condition mediated by MALT1 is cirrhosis (e.g., primary biliary cirrhosis or cirrhosis due to fatty liver disease, such as alcoholic and nonalcoholic steatosis). In certain embodiments, said disease or condition mediated by MALT1 is radiation-induced fibrosis (e.g., head and neck, gastrointestinal or pulmonary). In certain embodiments, said disease or condition mediated by MALT1 is primary sclerosing cholangitis. In certain embodiments, said disease or condition mediated by MALT1 is restenosis. In certain embodiments, said disease or condition mediated by MALT1 is cardiac fibrosis (e.g., endomyocardial fibrosis or atrial fibrosis). In certain embodiments, said disease or condition mediated by MALT1 is ophthalmic scarring. In certain embodiments, said disease or condition mediated by MALT1 is fibrosclerosis. In certain embodiments, said disease or condition mediated by MALT1 is a fibrotic cancer. In certain embodiments, said disease or condition mediated by MALT1 is fibroids. In certain embodiments, said disease or condition mediated by MALT1 is fibroma. In certain embodiments, said disease or condition mediated by MALT1 is a fibroadenoma. In certain embodiments, said disease or condition mediated by MALT1 is a fibrosarcoma. In certain embodiments, said disease or condition mediated by MALT1 is transplant arteriopathy. In certain embodiments, said disease or condition mediated by MALT1 is keloid. In certain embodiments, said disease or condition mediated by MALT1 is mediastinal fibrosis. In certain embodiments, said disease or condition mediated by MALT1 is myelofibrosis. In certain embodiments, said disease or condition mediated by MALT1 is retroperitoneal fibrosis. In certain embodiments, said disease or condition mediated by MALT1 is progressive massive fibrosis. In certain embodiments, said disease or condition mediated by MALT1 is nephrogenic systemic fibrosis.

In certain embodiments, said disease or condition mediated by MALT1 is obesity. In certain embodiments, said disease or condition mediated by MALT1 is steroid-resistance. In certain embodiments, said disease or condition mediated by MALT1 is glucose intolerance. In certain embodiments, said disease or condition mediated by MALT1 is metabolic syndrome.

In certain embodiments, said disease or condition mediated by MALT1 is atherosclerosis. In certain embodiments, said disease or condition mediated by MALT1 is restenosis of an atherosclerotic coronary artery. In certain embodiments, said disease or condition mediated by MALT1 is acute coronary syndrome. In certain embodiments, said disease or condition mediated by MALT1 is myocardial infarction. In certain embodiments, said disease or condition mediated by MALT1 is cardiac-allograft vasculopathy. In certain embodiments, said disease or condition mediated by MALT1 is stroke. In certain embodiments, said disease or condition mediated by MALT1 is a central nervous system disorder with an inflammatory or apoptotic component. In certain embodiments, said disease or condition mediated by MALT1 is Alzheimer's disease. In certain embodiments, said disease or condition mediated by MALT1 is Parkinson's disease. In certain embodiments, said disease or condition mediated by MALT1 is Huntington's disease. In certain embodiments, said disease or condition mediated by MALT1 is amyotrophic lateral sclerosis. In certain embodiments, said disease or condition mediated by MALT1 is spinal cord injury. In certain embodiments, said disease or condition mediated by MALT1 is neuronal ischemia. In certain embodiments, said disease or condition mediated by MALT1 is peripheral neuropathy.

In certain embodiments, said disease or condition mediated by MALT1 is a disease or disorder associated with a coronavirus (e.g., SARS-COV-2). In certain embodiments, said coronavirus is SARS-COV-2. In certain embodiments, the disease or disorder associated with SARS-COV-2 is COVID-19.

In certain embodiments, the disease or condition mediated by MALT1 is a rheumatic disease. In certain embodiments, the disease or condition mediated by MALT1 is an inflammatory arthropathy. In certain embodiments, the disease or condition mediated by MALT1 is rheumatoid arthritis, juvenile arthritis, Still's disease, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile Idiopathic Arthritis (JIA), juvenile psoriatic arthritis, juvenile rheumatoid arthritis, systemic onset juvenile rheumatoid arthritis, giant cell arteritis, secondary osteoarthritis from an inflammatory disease.

In certain embodiments, the disease or condition mediated by MALT1 is a connective tissue disease. In certain embodiments, the disease or condition mediated by MALT1 is lupus, systemic lupus erythematosus, juvenile systemic lupus erythematosus, nephritis, Sjögren's syndrome, scleroderma (systemic sclerosis), Raynaud's phenomenonjuvenile scleroderma, polymyositis, dermatomyositis, polymyositis-dermatomyositis, polymyalgia rheumatica, a mixed connective tissue disease, sarcoidosis, fibromyalgia, vasculitis microscopic polyangiitis, vasculitis, eosinophilic granulomatosis with polyangiitis (formerly known as Churg-Strauss Syndrome), granulomatosis with polyangiitis (formerly known as Wegener's granulomatosis), polyarteritis nodosa, Henoch-Schönlein purpura, idiopathic thrombocytopenia thrombotic purpura, juvenile vasculitis, polyarteritis nodossa (also known as panarteritis nodosa, periarteritis nodosa Kussmaul disease, Kussmaul-Maier disease or PAN), serum sickness, myasthenia gravis, Takayasu's arteritis, Behçet's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, primary biliary sclerosis, autoimmune hepatitis, chronic aggressive hepatitis, nonalcoholic hepatic steatosis, sclerosing cholangitis, membranous glomerulopathy, polymyositis, myositis, atherosclerosis, autoimmune hemolytic anemia, autoimmune orchitis, Goodpasture's disease,

In certain embodiments, the disease or condition mediated by MALT1 is a neurodegenerative disease or neuroinflammatory disease. In certain embodiments, the disease or condition mediated by MALT1 is multiple sclerosis, amyotropic lateral sclerosis, Guillain-Barre disease, autoimmune encephalomyelitis, Alzheimer's disease, major depressive disorder, traumatic brain injury, epilepsy, Parkinson's disease, or bipolar disorder.

In certain embodiments, the disease or condition mediated by MALT1 is an inflammatory bowel disease. In certain embodiments, the disease or condition mediated by MALT1 is Crohn's disease, ulcerative colitis, Celiac Sprue, Celiac disease, proctitis, eosinophilic gastroenteritis, autoimmune atrophic gastritis of pernicious anemia, or mastocytosis.

In certain embodiments, the disease or condition mediated by MALT1 is a skin autoimmune disorder. In certain embodiments, the disease or condition mediated by MALT1 is psoriasis. In certain embodiments, the disease or condition mediated by MALT1 is eczema. In certain embodiments, the disease or condition mediated by MALT1 is plaque psoriasis, Guttate psoriasis, psoriatic epidermal hyperplasia, inverse psoriasis, pustular psoriasis, erythrodermic psoriasis, atopic dermatitis, eczema dermatitis, dermatitis, rosacea, pruritus, alopecia areata, vitiligo, epidermal hyperplasia, juvenile dermatomyositis, dermatomyositis, or hidradenitis suppurativa.

In certain embodiments, the disease or condition mediated by MALT1 is an organ or cell transplant rejection. In certain embodiments, the disease or condition mediated by MALT1 is graft-versus-host disease. In certain embodiments, the disease or condition mediated by MALT1 is chronic graft-versus-host disease, acute graft-versus-host disease, or organ or cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In certain embodiments, the disease or condition mediated by MALT1 is an autoimmune disease of the eye. In certain embodiments, the disease or condition mediated by MALT1 is Graves' disease, noninfectious uveitis, dry eye syndrome, sympathetic ophthalmia, Cogan's syndrome, keratoconjunctivitis, vernal conjunctivitis, uveitis (e.g., uveitis associated with Behcet's disease and lens-induced uveitis), keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, or ocular neovascularization

In certain embodiments, the disease or condition mediated by MALT1 is an ocular manifestation of an autoimmune disease.

In certain embodiments, the disease or condition mediated by MALT1 is a respiratory disease. In certain embodiments, the disease or condition mediated by MALT1 is asthma, chronic obstructive pulmonary disease, or acute respiratory disease.

In certain embodiments, the disease or condition mediated by MALT1 is diabetes. In certain embodiments, the disease or condition mediated by MALT1 is Type I diabetes mellitus, Type II diabetes mellitus, or juvenile onset diabetes.

### Additional Methods

Another aspect of the invention provides methods of inhibiting cell proliferation in a subject by administering to the subject a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I), or inhibiting cell proliferation in a biological sample by contacting the biological sample with a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I). In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above. In certain embodiments, cell proliferation is inhibited for T-cells. In certain embodiments, cell proliferation is inhibited for B-cells. In certain embodiments, cell proliferation is inhibited for T-cells and B-cells.

Another aspect of the invention provides methods of inducing apoptosis of a cell in a subject by administering to the subject a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, lg-1, Ih-1, Ii-1, Ij-1, Ik-1, Il-1, Im-1, or In-1, or other compounds in Section I), or inducing apoptosis of a cell in a biological sample by contacting the biological sample with a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I). In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above. In certain embodiments, cell is a tumor cell. In certain embodiments, the cell is a lymphocyte. In certain embodiments, the cell is a T-cell. In certain embodiments, the cell is a B-cell.

Another aspect of the invention provides methods of inhibiting adhesion of a cell in a subject by administering to the subject a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I), or inhibiting adhesion of a cell in a biological sample by contacting the biological sample with a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, II-1, Im-1, or In-1, or other compounds in Section I). In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, lg-1, Ih-1, Ii-1, Ij-1, Ik-1, Il-1, Im-1, or In-1, or other compounds in Section I, or defined by one of the embodiments described above. In certain embodiments, the cell is a tumor cell. In certain embodiments, the cell is a lymphocyte. In certain embodiments, the cell is a T-cell. In certain embodiments, the cell is a B-cell.

Another aspect of the invention provides methods of inhibiting activation of T-cells or B-cells in a subject by administering to the subject a compound described herein (e.g., a compound of Formula I, I-1, II, III, Ia, Ib, Ic, Id, Ie, If, Ig, Ih, Ii, Ij, Ik, II, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I), or inhibiting activation of T-cells or B-cells in a biological sample by contacting the biological sample with a compound described herein (e.g., a compound of Formula I, I-1. II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, II-1, Im-1, or In-1, or other compounds in Section I). In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above.

Another aspect of the invention provides methods of inhibiting the activity of mucosa-associated lymphoid tissue lymphoma translation protein 1 (MALT1) or a MALT1 fusion protein in a subject by administering to the subject a compound described herein (e.g., a compound of Formula I, I-1, II, III, Ia, Ib, Ic, Id, Ie, If, Ig. Ih, Ii, Ij, Ik, II, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I), or inhibiting the activity of mucosa-associated lymphoid tissue lymphoma translation protein 1 (MALT1) or a MALT1 fusion protein in a biological sample by contacting the biological sample with a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I). In certain embodiments, the compound is a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I, or defined by one of the embodiments described above. In certain embodiments, the method inhibits the protease activity of MALT1. In certain embodiments, the method inhibits the protease activity of a MALT1 fusion protein (e.g., API2-MALT1). In certain embodiments, the method inhibits the protease activity of MALT1 or a MALT1 fusion protein for cleavage of a peptide substrate. In certain embodiments, the peptide substrate is A20, Bcl10, RelB, CYLD, NIK, regnase-1, roquin-1, roquin-2, LIMA1α, or MALT1. The inhibitor may selectively inhibit the protease activity of MALT1 or a MALT1 fusion protein for cleavage of a first peptide substrate over protease activity for cleavage of a second peptide substrate. In certain embodiments, the first and/or second substrate is A20, Bcl10, RelB, CYLD, NIK, regnase-1, roquin-1, roquin-2, LIMA1α, or MALT1. In certain embodiments, the selectivity is between about 1.25 fold and about 5 fold. In certain embodiments, the selectivity is between about 5 fold and about 10 fold. In certain embodiments, the selectivity is between about 10 fold and about 25 fold. In certain embodiments, the selectivity is between about 25 fold and about 50 fold. In certain embodiments, the selectivity is between about 50 fold and about 100 fold. In certain embodiments, the selectivity is between about 100 fold and about 250 fold. In certain embodiments. In certain embodiments, the selectivity is between about 250 fold and about 500 fold. In certain embodiments, the selectivity is between about 500 fold and about 1000 fold. In certain embodiments, or at least about 1000 fold.

### III. Combination Therapy

Another aspect of the invention provides for combination therapy. Pyrazolylcarboxamide compounds described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) or their pharmaceutically acceptable salts may be used in combination with additional therapeutic agents to treat diseases or conditions, such as an inflammatory disorder.

Accordingly, in some embodiments, the present invention provides a method of treating a disclosed disease or condition comprising administering to a patient in need thereof an effective amount of a compound disclosed herein and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents.

One or more other therapeutic agents may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another.

In certain embodiments, the compounds of the disclosure can be administered with one or more of a second therapeutic agent, sequentially or concurrently, either by the same route or by different routes of administration. When administered sequentially, the time between administrations is selected to benefit, among others, the therapeutic efficacy and/or safety of the combination treatment. In certain embodiments, the compound of the disclosure can be administered first followed by a second therapeutic agent, or alternatively, the second therapeutic agent administered first followed by the compound of the disclosure. In certain embodiments, the compound of the disclosure can be administered for the same duration as the second therapeutic agent, or alternatively, for a longer or shorter duration as the second therapeutic compound.

When administered concurrently, the compounds of the disclosure can be administered separately at the same time as the second therapeutic agent, by the same or different routes, or administered in a single composition by the same route. In certain embodiments, the compound of the disclosure is prepared as a first pharmaceutical composition, and the second therapeutic agent prepared as a second pharmaceutical composition, where the first pharmaceutical composition and the second pharmaceutical composition are administered simultaneously, sequentially, or separately. In certain embodiments, the amount and frequency of administration of the second therapeutic agent can used standard dosages and standard administration frequencies used for the particular therapeutic agent. See, e.g., Physicians' Desk Reference, 70th Ed., PDR Network, 2015; incorporated herein by reference.

In certain embodiments, the additional therapeutic agent is a leukotriene inhibitor, non-steroidal anti-inflammatory drug (NSAID), steroid, tyrosine kinase inhibitor, receptor kinase inhibitor, modulator of nuclear receptor family of transcription factor, HSP90 inhibitor, adenosine receptor (A2A) agonist, disease modifying antirheumatic drugs (DMARDS), phosphodiesterase (PDE) inhibitor, neutrophil elastase inhibitor, modulator of Axl kinase, an anti-cancer agent, anti-allergic agent, anti-nausea agent (or anti-emetic), pain reliever, cytoprotective agent, or a combination thereof. In certain embodiments, the additional therapeutic agent is an anti-cancer agent, an analgesic, an anti-inflammatory agent, or a combination thereof.

In certain embodiments, the second therapeutic agent is a leukotriene inhibitor. Examples of leukotriene inhibitors considered for use in combination therapies of the invention include but are not limited to montelukast, zafirlukast, pranlukast, zileuton, or combinations thereof.

In certain embodiments, the second therapeutic agent is a an NSAID. Examples of NSAIDs considered for use in combination therapies of the invention include but are not limited to acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbutazone, mefenamic acid, meclofenamic acid, flufenamic acid, tolfenamic acid, celecoxib, or combinations thereof.

In certain embodiments, the second therapeutic agent is a steroid. Examples of steroids considered for use in combination therapies of the invention include but are not limited to prednisone, prednisolone, methylprednisone, triacmcinolone, betamethasone, dexamethasone, and prodrugs thereof.

In certain embodiments, the second therapeutic agent is a tyrosine kinase inhibitor. Examples of tyrosine kinase inhibitors considered for use in combination therapies of the invention include but are not limited to inhibitors of the following kinases, including, among others: JAK, Syk, JNK/SAPK, MAPK, PI-3K, and/or Ripk2. In certain embodiments, the tyrosine kinase inhibitor is ruxolitinib, tofacitinib, oclactinib, filgotinib, ganotinib, lestaurtinib, momelotinib, pacritinib, upadacitinib, peficitinib, fedratinib, bentamapimod, D-JNKI-1 (XG-102, AM-111), ponatinib, WEHI-345, OD36, GSK583, idelalisib, copanlisib, taselisib, duvelisib, alpelisib, umbralisib, dactolisib, CUDC-907, entospletinib, fostamatinib, or combinations thereof.

In certain embodiments, the second therapeutic agent is a receptor kinase inhibitor, including among others, an inhibitor of EGFR or HER2. Examples of receptor kinase inhibitors considered for use in combination therapies of the invention include but are not limited to gefitinib, erlotinib, neratinib, lapatinib, cetuximab, panitumumab, vandetanib, necitumumab, osimertinib, trastuzumab, neratinib, lapatinib, pertuzumab, or combinations thereof.

In certain embodiments, the second therapeutic agent is a modulator of nuclear receptor family of transcription factors, including, among others, an inhibitor of PPAR, RXR, FXR, or LXR. In certain embodiments, the inhibitor is pioglitazone, bexarotene, obeticholic acid, ursodeoxycholic acid, fexaramine, hypocholamide, or combinations thereof.

In certain embodiments, the second therapeutic agent is an HSP90 inhibitor. Examples of HSP90 inhibitors considered for use in combination therapies of the invention include but are not limited to ganetespib, 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010, or combinations thereof.

In certain embodiments, the second therapeutic agent is an adenosine receptor 2A (A2A) agonist. Examples of adenosine receptor agonists considered for use in combination therapies of the invention include but are not limited to those disclosed in U.S. Pat. No. 9,067,963, which is incorporated herein by reference. In certain embodiments, the adenosine receptor agonist is LNC-3050, LNC-3015, LNC-3047, LNC-3052, or combinations thereof.

In certain embodiments, the second therapeutic agent is selected from disease modifying antirheumatic drugs (DMARDS). Examples of DMARDS considered for use in combination therapies of the invention include but are not limited to tocilizumab, certolizumab, etanercept, adalimumab, anakinra, abatacept, infliximab, rituximab, golimumab, uteskinumab, or combinations thereof.

In certain embodiments, the second therapeutic agent is a phosphodiesterase (PDE) inhibitor. Examples of phosphodiesterase inhibitor considered for use in combination therapies of the invention include but are not limited to apremilast, crisaborole, piclimilast, drotaverine, ibudulast, roflumilast, sildenafil, tadalafil, vardenafil, or combinations thereof.

In certain embodiments, the second therapeutic agent is a neutrophil elastase inhibitor. Examples of neutrophil elastase inhibitors considered for use in combination therapies of the invention include but are not limited to sivelestat.

In certain embodiments, the second therapeutic agent is a modulator of Axl kinase. Examples of modulators of Axl kinase considered for use in combination therapies of the invention include but are not limited to bemcentinib (BGB324 or R428), TP-0903, LY2801653, amuvatinib (MP-470), bosutinib (SKI-606), MGCD 265, ASP2215, cabozantinib (XI.184), forctinib (GSK1363089/XL880), and SGI-7079. In certain embodiments, the modulator of Axl kinase is a monoclonal antibody targeting AXL (e.g., YW327.6S2) or an AXL decoy receptor (e.g., GL2I.T), or glesatinib, merestinib, or a dual Flt3-Axl inhibitor such as gilteritinib.

In certain embodiments, the second therapeutic agent is a bispecific antibody, such as a bispecific antibody that binds to a tumor-specific antigen. Exemplary bispecific antibodies include but are not limited to Blincyto (blinatumomab), Kimmtrak (tebentafusp), Tecvayli (teclistamab), Lunsumio (mosunetuzumab), Epkinly (epcoritamab), and Columvi (glofitamab).

In certain embodiments, the second therapeutic agent is a chimeric antigen receptor (CAR) T-cell therapy. Exemplary CAR T-cell therapies include but are not limited to ABECMA® (idecabtagene vicleucel), BREYANZI® (lisocabtagene maraleucel), CARVYKTI™ (ciltacabtagene autoleucel), KYMRIAH™ (tisagenlecleucel), TECARTUS™ (brexucabtagene autoleucel), and YESCARTA™ (axicabtagene ciloleucel).

In certain embodiments, the additional therapeutic agent is an anti-cancer agent or chemo-therapeutic agent. Examples of anti-cancer agents considered for use in combination therapies of the invention include but are not limited erlotinib, bortezomib, fulvestrant, sunitib, imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adezelesin, anthracyclin, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine, and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, edrecolomab, epirubicin, bleomycin, plicamycin, mitomycin, as well as other anticancer antibodies (cetuximab, bevacizumab, ibritumomab, abagovomab, adecatumumab, afutuzumab, alacizumab, alemtuzumab, anatumomab, apolizumab, bavituximab, belimumab, bivatuzumab mertansine, blinatumomab, brentuximab vedotin, cantuzumab mertansine, catumazomab, cetuximab, citatuzumab bogatox, cixutumumab, clivatuzumab tetraxetan, conatumumab, dacetuzumab, daclizumab, detumomab, ecromeximab, edrecolomab, elotuzumab, epratuzumab, ertumaxomab, etaracizumab, farletuzumab, figitumumab, fresolimumab, galiximab, gembatumumab vedotin, gemtuzumab, ibritumomab tiuxetan, inotuzumab ozogamicin, intetumumab, ipilimumab, iratumumab, labetuzumab, lexatumumab, lintuzumab, lucatumumab, lumilisimab, mapatumumab, matuzumab, milatuzumab, mitumomab, nacolomab tafenatox, naptumomab estafenatox, necitumumab, nimotuzumab, ofatumumab, olaratumab, oportuzumab monatox, oregovomab, panitumumab, pemtumomab, pertuzumab, pintumomab, pritumumab, ramucirumab, rilotumumab, robatumumab, rituximab, sibrotuzumab, tacatuzumab tetraxetan, taplitumomab paptox, tenatumomab, ticilimumab, tigatuzumab, tositumomab or ¹³¹I-tositumomab, trastuzumab, tremelimumab, tuocotuzumab celmoleukin, veltuzumab, visilizumab, volocixumab, votumumab, zalutumumab, zanolimumab, IGN-101, MDX-010, ABX-EGR, EMD72000, ior-t1, MDX-220, MRA, H-11 scFv, huJ591, TriGem, TriAb, R3, MT-201, G-250, ACA-125, Onyvax-105, CD:-960, Cea-Vac, BrevaRex AR54, IMC-1C11, GlioMab-H, ING-1, anti-LCG MAbs, MT-103, KSB-303, Therex, KW2871, anti-HMI.24, Anti-PTHrP, 2C4 antibody, SGN-30, TRAIL-RI MAb, Prostate Cancer antibody, H22xKi-r, ABX-Mai, Imuteran, Monopharm-C), and antibody-drug conjugates comprising any of the above agents (especially auristatins MMAE and MMAF, maytansinoids like DM-1, calicheamycins, or various cytotoxins).

In certain embodiments, the additional therapeutic agent is selected from anastrozole (ARIMIDEX®), bicalutamide (CASODEX®), bleomycin sulfate (BLENOXANE®), busulfan (MYLERAN®), busulfan injection (BUSULFEX®), capecitabine (XELODA®), N4-pentoxycarbonyl-5-deoxy-5-fluorocytidine, carboplatin (PARAPLATIN®), carmustine (BiCNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-Dome®), dactinomycin (actinomycin D, COSMEGAN®), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCIL®, EFUDEX®), flutamide (EULEXIN®), tezacitibine, gemcitabine (difluorodeoxycitidine), hydroxyurea (HYDREA®), idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), gemtuzumab ozogamicin (MYLOTARG™), paclitaxel (TAXOL®), nab-paclitaxel (ABRAXANE®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®).

In certain embodiments, the additional therapeutic agent is capable of inhibiting BRAF, MEK, CDK4/6, SHP-2, HDAC, EGFR, MET, mTOR, PI3K or AKT, or a combination thereof. In a particular embodiment, the compounds of the present invention are combined with another therapeutic agent selected from vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, BYL719 or CLR457, or a combination thereof.

In certain embodiments, the additional therapeutic agent is selected based on the disease or condition that is being treated. For example, in the treatment of melanoma, the additional therapeutic agent is selected from aldesleukin (e.g., PROLEUKIN®), dabrafenib (e.g., TAFINLAR®), dacarbazine, recombinant interferon alfa-2b (e.g., INTRON® A), ipilimumab, trametinib (e.g., MEKINIST®), peginterferon alfa-2b (e.g., PEGINTRON®, SYLATRON™), vemurafenib (e.g., ZELBORAF®)), and ipilimumab (e.g., YERVOY®).

For the treatment of ovarian cancer, the additional therapeutic agent is selected from doxorubicin hydrochloride (Adriamycin®), carboplatin (PARAPLATIN®), cyclophosphamide (CYTOXAN®, NEOSAR®), cisplatin (PLATINOL®, PLATINOL-AQ®), doxorubicin hydrochloride liposome (DOXIL®, DOX-SL®, EVACET®, LIPODOX®), gemcitabine hydrochloride (GEMZAR®), topotecan hydrochloride (HYCAMTIN®), and paclitaxel (TAXOL®).

For the treatment of thyroid cancer, the additional therapeutic agent is selected from doxorubicin hydrochloride (Adriamycin®), cabozantinib-S-malate (COMETRIQ®), and vandetanib (CAPRELSA®).

For the treatment of colon cancer, the additional therapeutic agent is selected from fluorouracil (e.g., ADRUCIL®, EFUDEX®, FLUOROPLEX®), bevacizumab (AVASTIN®), irinotecan hydrochloride (CAMPTOSTAR®), capecitabine (XELODA®), cetuximab (ERBITUX®), oxaliplatin (ELOXATIN®), leucovorin calcium (WELLCOVORIN®), regorafenib (STIVARGA®), panitumumab (VECTIBIX®), and ziv-aflibercept (ZALTRAP®).

For the treatment of lung cancer, the additional therapeutic agent is selected from methotrexate, methotrexate LPF (e.g., FOLEX®, FOLEX PFS®, Abitrexate®, MEXATE®, MEXATE-AQ®), paclitaxel (TAXOL®), paclitaxel albumin-stabilized nanoparticle formulation (ABRAXANE®), afatinib dimaleate (GILOTRIF®), pemetrexed disodium (ALIMTA®), bevacizumab (AVASTIN®), carboplatin (PARAPLATIN®), cisplatin (PLATINOL®, PLATINOL-AQ®), crizotinib (XALKORI®), erlotinib hydrochloride (TARCEVA®), gefitinib (IRESSA®), and gemcitabine hydrochloride (GEMZAR®).

For the treatment of pancreatic cancer, the other therapeutic agent may be selected from fluorouracil (ADRUCIL®), EFUDEX®, FLUOROPLEX®), erlotinib hydrochloride (TARCEVA®), gemcitabine hydrochloride (GEMZAR®), and mitomycin or mitomycin C (MITOZYTREX™, MUTAMYCIN®).

For the treatment of cervical cancer, the additional therapeutic agent is selected from bleomycin (BLENOXANE®), cisplatin (PLATINOL®, PLATINOL-AQ®) and topotecan hydrochloride (HYCAMTIN®).

For the treatment of head and neck cancer, the additional therapeutic agent is selected from methotrexate, methotrexate LPF (e.g., FOLEX®, FOLEX PFS®, Abitrexate®, MEXATE®, MEXATE-AQ®), fluorouracil (ADRUCIL®, EFUDEX®, FLUOROPLEX®), bleomycin (BLENOXANE®), cetuximab (ERBITUX®), cisplatin (PLATINOL®, PLATINOL-AQ®) and docetaxel (TAXOTERE®).

For the treatment of leukemia, including chronic myelomonocytic leukemia (CMML), the additional therapeutic agent is selected from bosutinib (BOSULIF®), cyclophosphamide (CYTOXAN®, NEOSAR®), cytarabine (CYTOSAR-U®, TARABINE PFS®), dasatinib (SPRYCEL®), imatinib mesylate (GLEEVEC®), ponatinib (ICLUSIG®), nilotinib (TASIGNA®) and omacetaxine mepesuccinate (SYNRIBO®).

In some instances, patients may experience allergic reactions to the compounds of the present invention and/or other anti-cancer agent(s) during or after administration. Therefore, anti-allergic agents may be administered to minimize the risk of an allergic reaction. Suitable anti-allergic agents include corticosteroids, such as dexamethasone (e.g., DECADRON®), beclomethasone (e.g., BECLOVENT®), hydrocortisone (also known as cortisone, hydrocortisone sodium succinate, hydrocortisone sodium phosphate; e.g., ALA-CORT®, hydrocortisone phosphate, Solu-CORTEF®, HYDROCORT Acetate® and LANACORT®), prednisolone (e.g., DELTA-Cortel®, ORAPRED®, PEDIAPRED® and PRELONE®), prednisone (e.g., DELTASONE®, LIQUID RED®, METICORTEN® and ORASONE®), methylprednisolone (also known as 6-methylprednisolone, methylprednisolone acetate, methylprednisolone sodium succinate; e.g., DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

In other instances, patients may experience nausea during and after administration of the compound of the present invention and/or other anti-cancer agent(s). Therefore, anti-emetics may be administered in preventing nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and Zunrisa®), and combinations thereof.

In yet other instances, medication to alleviate the pain experienced during the treatment period is prescribed to make the patient more comfortable. Common over-the-counter analgesics, such TYLENOL®, are often used. Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) are also useful for moderate or severe pain.

Furthermore, cytoprotective agents (such as neuroprotectants, free-radical scavengers, cardioprotectors, anthracycline extravasation neutralizers, nutrients and the like) may be used as an adjunct therapy to protect normal cells from treatment toxicity and to limit organ toxicities. Suitable cytoprotective agents include amifostine (ETHYOL. ®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In yet another aspect, a compound of the present invention may be used in combination with known therapeutic processes, for example, with the administration of hormones or in radiation therapy. In certain instances, a compound of the present invention may be used as a radiosensitizer, especially for the treatment of tumors which exhibit poor sensitivity to radiotherapy.

The doses and dosage regimen of the active ingredients used in the combination therapy may be determined by an attending clinician. In certain embodiments, the compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) and the additional therapeutic agent(s) are administered in doses commonly employed when such agents are used as monotherapy for treating the disease or condition. In other embodiments, the compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) and the additional therapeutic agent(s) are administered in doses lower than the doses commonly employed when such agents are used as monotherapy for treating the disease or condition. In certain embodiments, the compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) and the additional therapeutic agent(s) are present in the same composition, which is suitable for oral administration.

In certain embodiments, the compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of a compound described herein (e.g., a compound of Formula I, I-1, II, III, Ia, Ib, Ic, Id, Ie, If, Ig, Ih, Ii, Ij, Ik, II, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I), a pharmaceutically acceptable carrier, vehicle or diluent, and optionally at least one additional therapeutic agent listed above. In certain embodiments, the kit further comprises instructions, such as instructions for treating a disease described herein.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I, I-1, II, III, Ia, Ib, Ic, Id, Ie, If, Ig, Ih, Ii, Ij, Ik, Il, Im, In, Ia-1, Ib-1, Ic-1, Id-1, Ie-1, If-1, Ig-1, Ih-1, Ii-1, Ij-1, Ik-1, Il-1, Im-1, or In-1, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (e.g., capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a imidazopyrimidine compound or related compound described herein in a therapeutically effective amount for the treatment of a disease or condition described herein.

### IV. Medical Kits

Another aspect of the invention provides a medical kit comprising, for example, (i) a compound described herein, and (ii) instructions for use according to a method described herein.

### V. Enumerated Embodiments

The following exemplary embodiments are provided:

Embodiment 1 provides a compound of formula I-1:

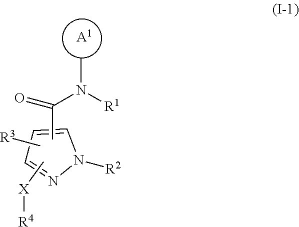

or a pharmaceutically acceptable salt thereof; wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur;
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²,
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

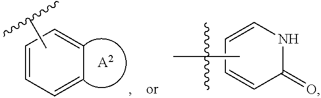

wherein the phenyl, heteroaryl,

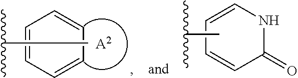

are substituted with t occurrences of R⁷;
R⁵ is a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹;
R¹ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—; and
m, n, q, t, and y are independently 0, 1, or 2;
provided that R² and R³ are not simultaneously C₁₋₄ alkyl.

Embodiment 2 provides the compound of embodiment 1, wherein the compound is a compound of Formula I-1.

Embodiment 3 provides the compound of embodiment 1 or 2, wherein R³ is C₁₋₆ haloalkyl.

Embodiment 4 provides the compound of embodiment 1 or 2, wherein R³ is —CF₃.

Embodiment 5 provides the compound of embodiment 1 or 2, wherein R³ is C₁₋₆ alkyl.

Embodiment 6 provides the compound of embodiment 1 or 2, wherein R³ is methyl.

Embodiment 7 provides the compound of any one of embodiments 1-6, wherein the compound is a compound of Formula Ia-1 or a pharmaceutically acceptable salt thereof:

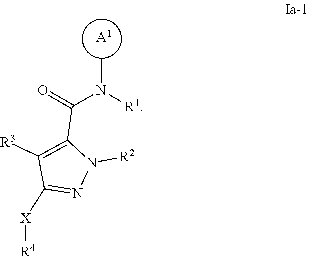

Embodiment 8 provides the compound of any one of embodiments 1-6, wherein the compound is a compound of Formula Ib-1 or a pharmaceutically acceptable salt thereof:

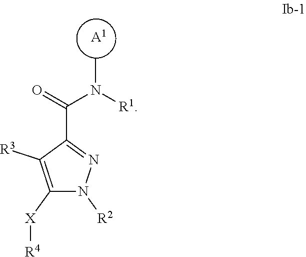

Embodiment 9 provides the compound of any one of embodiments 1-8, wherein R¹ is hydrogen.

Embodiment 10 provides the compound of any one of embodiments 1-8, wherein R¹ is C₁₋₄ alkyl.

Embodiment 11 provides the compound of any one of embodiments 1-10, wherein X is a bond.

Embodiment 12 provides the compound of any one of embodiments 1-10, wherein X is C₁₋₄ alkylene or C₃₋₅ cycloalkylene.

Embodiment 13 provides the compound of any one of embodiments 1-10, wherein X is C₂₋₄ alkenylene, —(C₀₋₂ alkylene)-O—(C₀₋₂ alkylene), or —(C₀₋₂ alkylene)-N(R⁹)—(C₀₋₂ alkylene).

Embodiment 14 provides the compound of any one of embodiments 1-13, wherein A¹ is pyridinyl substituted with m occurrences of R⁵ and n occurrences of R⁶.

Embodiment 15 provides the compound of embodiment 1, wherein the compound is a compound of Formula Ic-1 or a pharmaceutically acceptable salt thereof:

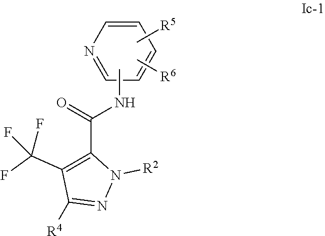

Embodiment 16 provides the compound of embodiment 1, wherein the compound is a compound of Formula Id-1 or a pharmaceutically acceptable salt thereof:

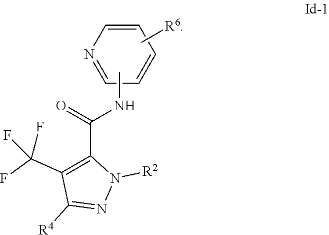

Embodiment 17 provides the compound of embodiment 1, wherein the compound is a compound of Formula Ie-1 or a pharmaceutically acceptable salt thereof:

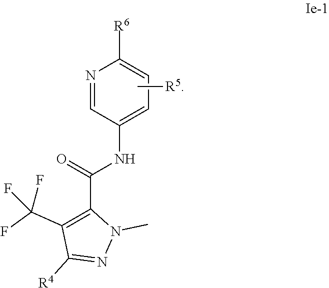

Embodiment 18 provides the compound of embodiment 1, wherein the compound is a compound of Formula If-1 or a pharmaceutically acceptable salt thereof:

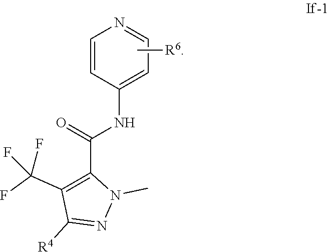

Embodiment 19 provides the compound of any one of embodiments 1-18, wherein R⁴ is phenyl substituted with t occurrences of R⁷.

Embodiment 20 provides the compound of any one of embodiments 1-18, wherein R⁴ is a 5-6 membered heteroaryl containing 1, 2, or 3 heteroatoms independently selected from oxygen, nitrogen, and sulfur, wherein the heteroaryl is substituted with t occurrences of R⁷.

Embodiment 21 provides the compound of any one of embodiments 1-18, wherein R⁴ is pyridinyl substituted with t occurrences of R⁷.

Embodiment 22 provides the compound of any one of embodiments 1-18, wherein R⁴ is a 9-10 membered bicyclic heteroaryl containing 1, 2, or 3 heteroatoms independently selected from oxygen, nitrogen, and sulfur, wherein the heteroaryl is substituted with t occurrences of R⁷.

Embodiment 23 provides the compound of any one of embodiments 1-18, wherein R⁴ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷.

Embodiment 24 provides the compound of any one of embodiments 1-18, wherein R⁴ is

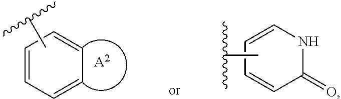

each of which are substituted with t occurrences of R⁷.

Embodiment 25 provides the compound of embodiment 1, wherein the compound is a compound of Formula Ig-1, Ih-1, Ii-1, or Ij-1 or a pharmaceutically acceptable salt thereof:

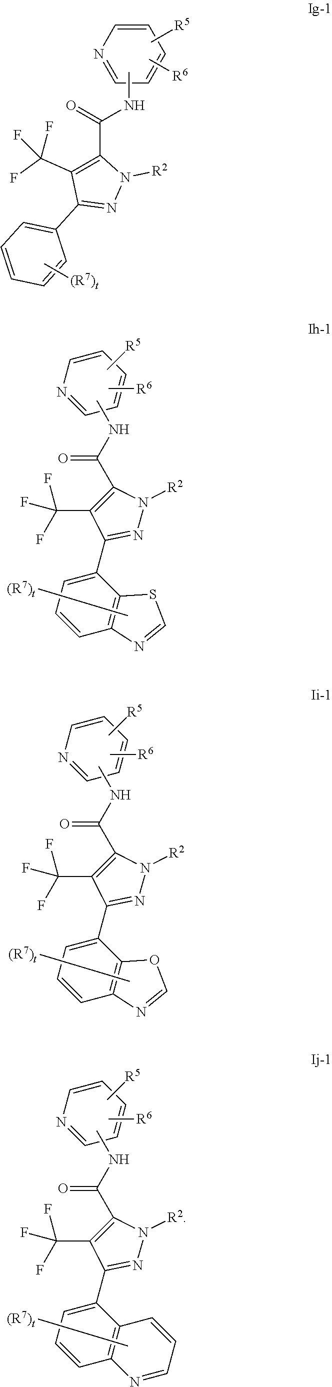

Embodiment 26 provides the compound of embodiment 1, wherein the compound is a compound of Formula Ik-1, II-1, Im-1, or In-1 or a pharmaceutically acceptable salt thereof:

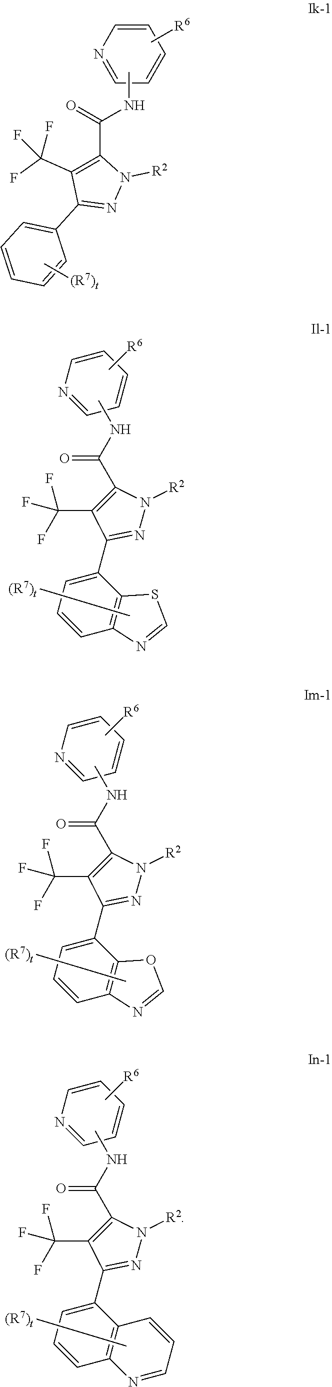

Embodiment 27 provides the compound of any one of embodiments 1-26, wherein R⁷ represents independently for each occurrence halo, hydroxyl, C₁₋₆ alkyl, C₁₋₆ haloalkyl, C₁₋₆ hydroxyalkyl, C₁₋₆ alkoxyl, or C₃₋₇ cycloalkyl.

Embodiment 28 provides the compound of any one of embodiments 1-26, wherein R⁷ represents independently for each occurrence halo, C₁₋₆ alkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, or —N(R⁹)(R¹⁰).

Embodiment 29 provides the compound of any one of embodiments 1-26, wherein R⁷ represents independently for each occurrence halo, C₁₋₆ alkyl, —(C₀₋₄ alkylene)-C(O)R⁸, —C(O)N(R⁹)(R¹⁰), —N(R⁹)C(O)R¹¹, —SO₂R¹¹, or —O-A³.

Embodiment 30 provides the compound of any one of embodiments 1-29, wherein t is 1.

Embodiment 31 provides the compound of any one of embodiments 1-26, wherein t is 0.

Embodiment 32 provides the compound of any one of embodiments 1-31, wherein R² is C₁₋₆ alkyl.

Embodiment 33 provides the compound of any one of embodiments 1-31, wherein R² is methyl.

Embodiment 34 provides the compound of any one of embodiments 1-33, wherein R⁵ is a 5 membered heteroaryl containing 1, 2, or 3 heteroatoms independently selected from oxygen, nitrogen, and sulfur, wherein the heteroaryl is substituted with q occurrences of R¹².

Embodiment 35 provides the compound of any one of embodiments 1-33, wherein R⁵ is a 1,2,3-triazolyl, pyrazolyl, oxazolyl, imidazolyl, isoxazolyl, pyrrolyl, or furanyl, each of which substituted with q occurrences of R¹².

Embodiment 36 provides the compound of any one of embodiments 1-33, wherein R⁵ is a 1,2,3-triazolyl substituted with q occurrences of R¹².

Embodiment 37 provides the compound of any one of embodiments 1-36, wherein q is 0.

Embodiment 38 provides the compound of any one of embodiments 1-37, wherein R⁶ represents independently for each occurrence halo, C₁₋₆ alkyl, or C₁₋₆ haloalkyl.

Embodiment 39 provides the compound of any one of embodiments 1-37, wherein R⁶ is chloro.

Embodiment 40 provides the compound of any one of embodiments 1-37, wherein R⁶ is C₁₋₆ haloalkyl.

Embodiment 41 provides the compound of any one of embodiments 1-37, wherein R⁶ is —CF₃.

Embodiment 42 provides a compound represented by Formula II-1:

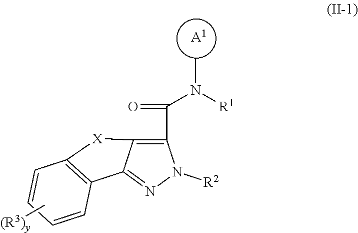

or a pharmaceutically acceptable salt thereof; wherein:
A¹ is a 6 membered heteroaryl containing 1 or 2 nitrogen atoms, wherein the heteroaryl is substituted with m occurrences of R⁵ and n occurrences of R⁶;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, or C₃₋₇ cycloalkyl;
R³ represents independently for each occurrence halo, hydroxyl, C₁₋₆ alkyl, C₁₋₆ haloalkyl, C₁₋₆ alkoxyl, or C₃₋₇ cycloalkyl;
R⁴ and R⁷ are independently hydrogen, C₁₋₆ alkyl, or C₃₋₇ cycloalkyl, or R⁴ and R⁷ are taken together with the nitrogen atom to which they are attached to form a 3-7 membered heterocyclic ring containing 1 nitrogen atom;
R⁵ is a 5-6 membered heteroaryl containing 1, 2, or 3 heteroatoms independently selected from oxygen, nitrogen, and sulfur, wherein the heteroaryl is substituted with q occurrences of R⁸;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, or —N(R⁴)(R⁷);
R⁸ represents independently for each occurrence halo, hydroxyl, C₁₋₆ alkyl, C₁₋₆ haloalkyl, C₁₋₆ alkoxyl, or C₃₋₇ cycloalkyl;
X is a C₁₋₇ bivalent straight or branched saturated hydrocarbon chain wherein 1 or 2 methylene units of the chain are independently and optionally replaced with O; and
m, n, and q are independently 0, 1, or 2.

Embodiment 43 provides the compound of embodiment 42, wherein the compound is a compound of Formula II-1.

Embodiment 44 provides the compound of embodiment 42 or 43, wherein A¹ is pyridinyl substituted with m occurrences of R⁵ and n occurrences of R⁶.

Embodiment 45 provides the compound of any one of embodiments 42-44, wherein R⁵ is a 5 membered heteroaryl containing 1, 2, or 3 heteroatoms independently selected from oxygen, nitrogen, and sulfur, wherein the heteroaryl is substituted with q occurrences of R⁸.

Embodiment 46 provides the compound of any one of embodiments 42-44, wherein R⁵ is 1,2,3-triazolyl, pyrazolyl, oxazolyl, imidazolyl, isoxazolyl, pyrrolyl, or furanyl, each of which substituted with q occurrences of R⁸.

Embodiment 47 provides the compound of any one of embodiments 42-44, wherein R⁵ is 1,2,3-triazolyl substituted with q occurrences of R⁸.

Embodiment 48 provides the compound of any one of embodiments 42-47, wherein q is 0.

Embodiment 49 provides the compound of any one of embodiments 42-48, wherein m is 1.

Embodiment 50 provides the compound of any one of embodiments 42-44, wherein m is 0.

Embodiment 51 provides the compound of any one of embodiments 42-50, wherein n is 1.

Embodiment 52 provides the compound of any one of embodiments 42-51, wherein R⁶ represents independently for each occurrence halo, C₁₋₆ alkyl, or C₁₋₆ haloalkyl.

Embodiment 53 provides the compound of any one of embodiments 42-51, wherein R⁶ is chloro.

Embodiment 54 provides the compound of any one of embodiments 42-51, wherein R⁶ is C₁₋₆ haloalkyl.

Embodiment 55 provides the compound of any one of embodiments 42-51, wherein R⁶ is —CF 3.

Embodiment 56 provides the compound of any one of embodiments 42-52, wherein R¹ is hydrogen

Embodiment 57 provides the compound of any one of embodiments 42-56, wherein R² is C₁₋₆ alkyl.

Embodiment 58 provides the compound of any one of embodiments 42-56, wherein R² is methyl.

Embodiment 59 provides the compound of any one of embodiments 42-58, wherein X is —CH₂CH₂— or —CH₂CH₂CH₂—.

Embodiment 60 provides the compound of any one of embodiments 42-58, wherein X is —CH₂CH₂CH₂— wherein one CH₂ is replaced with —O—.

Embodiment 61 provides a compound represented by Formula III-1:

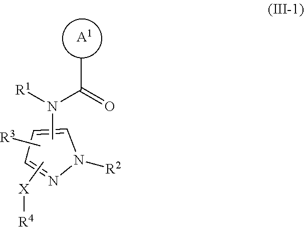

or a pharmaceutically acceptable salt thereof; wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur;
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

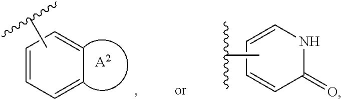

wherein the phenyl, heteroaryl,

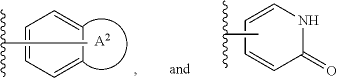

are substituted with t occurrences of R⁷;
R⁵ is a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹;
R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—; and
m, n, q, t, and y are independently 0, 1, or 2;
provided that R² and R³ are not simultaneously C₁₋₄ alkyl.

Embodiment 62 provides the compound of embodiment 61, wherein the compound is a compound of Formula III.

Embodiment 63 provides the compound of embodiment 61 or 62, wherein R³ is C₁₋₆ haloalkyl.

Embodiment 64 provides the compound of embodiment 61 or 62, wherein R³ is —CF₃.

Embodiment 65 provides the compound of embodiment 61 or 62, wherein R³ is C₁₋₆ alkyl.

Embodiment 66 provides the compound of embodiment 61 or 62, wherein R³ is methyl.

Embodiment 67 provides the compound of any one of embodiments 61-66, wherein A¹ is pyridinyl substituted with m occurrences of R⁵ and n occurrences of R⁶.

Embodiment 68 provides the compound of any one of embodiments 61-67, wherein R⁴ is phenyl substituted with t occurrences of R⁷.

Embodiment 69 provides the compound of any one of embodiments 61-67, wherein R⁴ is a 5-6 membered heteroaryl containing 1, 2, or 3 heteroatoms independently selected from oxygen, nitrogen, and sulfur, wherein the heteroaryl is substituted with t occurrences of R⁷.

Embodiment 70 provides the compound of any one of embodiments 61-67, wherein R⁴ is a 9-10 membered bicyclic heteroaryl containing 1, 2, or 3 heteroatoms independently selected from oxygen, nitrogen, and sulfur, wherein the heteroaryl is substituted with t occurrences of R⁷.

Embodiment 71 provides a compound in Table 1, 2, or 3, or a pharmaceutically acceptable salt thereof.

Embodiment 72 provides a pharmaceutical composition comprising a compound of any one of embodiments 1-71 and a pharmaceutically acceptable carrier.

Embodiment 73 provides a method for treating a disease or condition mediated by MALT1, comprising administering to a subject in need thereof a therapeutically effective amount of a compound of any one of embodiments 1-71 to treat the disease or condition.

Embodiment 74 provides the method of embodiment 73, wherein said disease or condition mediated by MALT1 is a proliferative disorder.

Embodiment 75 provides the method of embodiment 73, wherein said disease or condition mediated by MALT1 is an inflammatory disorder.

Embodiment 76 provides the method of embodiment 73, wherein said disease or condition mediated by MALT1 is an autoimmune disorder.

Embodiment 77 provides the method of embodiment 73, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

Embodiment 78 provides the method of embodiment 73, wherein said disease or condition mediated by MALT1 is cancer.

Embodiment 79 provides the method of embodiment 78, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

Embodiment 80 provides the method of embodiment 73, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

Embodiment 81 provides the method of embodiment 73, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenia thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythematosus, Hashimoto's thyroiditis, myasthenia gravis, nephrotic syndrome, eosinophilia fasciitis, hyper IgE syndrome, Jepromatous leprosy, sezary syndrome, idiopathic thrombocytopenia purpura, restenosis following angioplasty, a tumor, or artherosclerosis.

Embodiment 82 provides the method of embodiment 73, wherein said disease or condition mediated by MALT1 is allergic rhinitis, nasal inflammation, asthma, chronic obstructive pulmonary disease (COPD), bronchitis, emphysema, chronic eosinophilic pneumonia, adult respiratory distress syndrome, sinusitis, allergic conjunctivitis, idiopathic pulmonary fibrosis, atopic dermatitis, asthma, allergic rhinitis, arthritis, inflammatory bowel disease, Crohn's disease, ulcerative colitis, multiple sclerosis, endometriosis, eczema, psoriasis, rosacea, or lupus erythematosus.

Embodiment 83 provides the method of any one of embodiments 73-82, wherein the subject is a human.

Embodiment 84 provides a method of inhibiting the activity of MALT1, comprising contacting a MALT1 with an effective amount of a compound of any one of embodiments 1-71 to inhibit the activity of said MALT1.

### EXAMPLES

The invention now being generally described, will be more readily understood by reference to the following examples, which are included merely for purposes of illustrating certain aspects and embodiments of the present invention, and are not intended to limit the invention.

### Example 1—Synthesis of ethyl 3-bromo-1-methyl-4-(trifluoromethyl)-1H-pyrazole-5-carboxylate (INT 3)

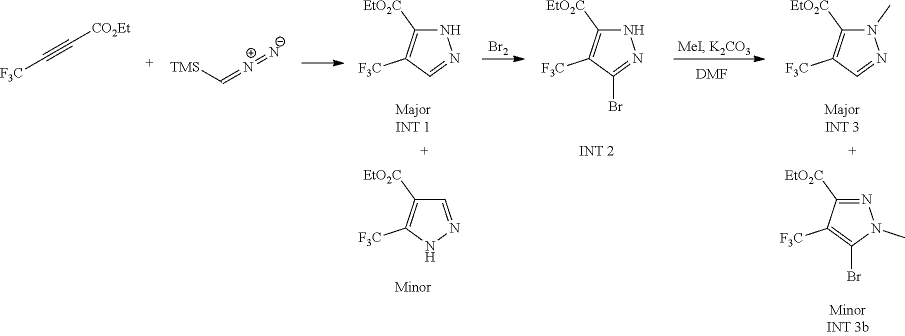

Step 1: Synthesis of ethyl 4-(trifluoromethyl)-1H-pyrazole-5-carboxylate (INT 1): 2 M (Trimethylsilyl)diazomethane solution in hexanes (9.0 mL, 18.1 mmol, 1.20 eq) was added dropwise to a stirred solution of ethyl 4,4,4-trifluoro-2-butynoate (2.2 mL, 15.1 mmol, 1.00 eq) in Et₂O (100 mL) under nitrogen at 0° C. The reaction mixture was stirred at 0° C. for 10 minutes, then allowed to warm to rt and stirred for 18 hours. The solvent was removed under reduced pressure and the crude residue was purified by column chromatography eluting with 20-50% EtOAc in cyclohexane to give a mixture of ethyl 4-(trifluoromethyl)-1H-pyrazole-5-carboxylate and ethyl 5-(trifluoromethyl)-1H-pyrazole-4-carboxylate (2,660 mg, 12.8 mmol, 85%) as a white solid (7.5:1 regioisomeric ratio). (ES, m/z): [M+H]⁺ 209.0. Major regioisomer (ethyl 4-(trifluoromethyl)-1H-pyrazole-5-carboxylate) (INT 1). ¹H NMR (400 MHz, CDCl₃) δ 14.28 (br s, 1H), 8.18 (s, 1H), 4.48 (q, J=7.1 Hz, 2H), 1.44 (t, J=7.1 Hz, 3H). Minor regioisomer (ethyl 5-(trifluoromethyl)-1H-pyrazole-4-carboxylate). ¹H NMR (400 MHz, CDCl₃) δ 13.25 (br s, 1H), 8.42 (s, 1H), 4.37 (q, J=7.1 Hz, 2H), 1.39 (t, J=7.1 Hz, 3H). Regioisomers separated by chiral SFC, stationary phase: YMC Amylose-C IPA; column: TORUS DEA 20×150 mm, 5 um; conditions: 5/95 IPA (0.1% NH₄OH)/CO₂, 100 mL/minutes, 120 bar, 40° C., DAD 240 nm; 2660 mg in 50 ml IPA (approx. 50 mg/ml), 1000 ul/INJECTION, 50 mg/inj, CT 4/0.5 min TOTAL INJECTIONS 50

Step 2: Synthesis of ethyl 3-bromo-4-(trifluoromethyl)-1H-pyrazole-5-carboxylate (INT 2): Bromine (591 μL, 11.5 mmol, 2.00 eq) was added to a stirred mixture of ethyl 4-(trifluoromethyl)-1H-pyrazole-5-carboxylate (1.2 g, 5.77 mmol, 1.00 eq) and NaOH (970 mg, 17.3 mmol, 3.00 eq) in DMF (10 mL), the reaction was stirred at 50° C. for 18 hours. The reaction was allowed to cool to rt and was diluted with EtOAc, water and brine. The phases were separated, and the aqueous layer was extracted with EtOAc. The combined organic layers were washed with saturated aqueous LiCl, saturated aqueous Na₂S₂O₃ and brine, dried through a phase separator and the solvent was removed under reduced pressure. The crude residue was purified by column chromatography eluting with 0-50% EtOAc in cyclohexane to give the title compound as a light yellow solid (1,532 mg, 5.34 mmol, 93%). (ES, m/z): [M+H]⁻ 286.8 and 288.8, ¹H NMR (400 MHz, CDCl₃) δ 11.25 (br s, 1H), 4.46 (q, J=7.1 Hz, 2H), 1.42 (t, J=7.1 Hz, 3H).

Step 3: Synthesis of ethyl 3-bromo-1-methyl-4-(trifluoromethyl)-1H-pyrazole-5-carboxylate (INT 3): K₂CO₃ (1.1 g, 8.01 mmol, 1.50 eq) was added to a solution of ethyl 3-bromo-4-(trifluoromethyl)-1H-pyrazole-5-carboxylate (1.53 g, 5.34 mmol, 1.00 eq) and MeI (1.7 mL, 26.7 mmol, 5.00 eq) in DMF (11 mL) and the reaction was stirred at 60° C. for 90 minutes. The reaction was diluted with EtOAc, water and brine. The aqueous phase was separated and extracted with EtOAc. The combined organic layers were washed with saturated aqueous LiCl, brine and dried through a phase separator. The solvent was removed under reduced pressure and the crude residue was purified by column chromatography eluting with 0-20% EtOAc in cyclohexane to give two regioisomers: ethyl 3-bromo-1-methyl-4-(trifluoromethyl)-1H-pyrazole-5-carboxylate (903 mg, 3.00 mmol, 56%) as an off-white solid, and ethyl 5-bromo-1-methyl-4-(trifluoromethyl)-1H-pyrazole-3-carboxylate (335 mg, 1.11 mmol, 21%), as a white solid. Major regioisomer (INT 3) (ES, m/z): [M+H]⁺ 300.8 and 302.8, ¹H NMR (400 MHz, CDCl₃) δ 4.42 (q, J=7.2 Hz, 2H), 4.11 (s, 3H), 1.40 (t, J=7.2 Hz, 3H). Minor regioisomer (INT 3b). (ES, m/z): [M+H]⁺ 300.8 and 302.8, ¹H NMR (400 MHz, CDCl₃) δ 4.43 (q, J=7.1 Hz, 2H), 4.00 (s, 3H), 1.40 (t, J=7.1 Hz, 3H).

### Example 2—Synthesis of 1-methyl-3-(thieno[2,3-c]pyridin-3-yl)-4-(trifluoromethyl)-N-(2-(trifluoromethyl)pyridin-4-yl)-1H-pyrazole-5-carboxamide (I-1)

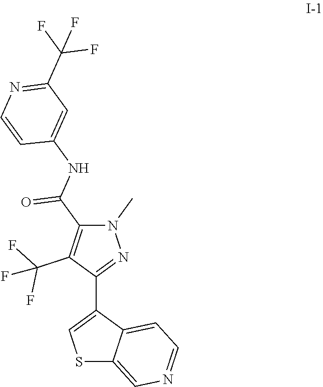 [SMILES: Cn1nc(-c2csc3cnccc23)c(C(F)(F)F)c1C(=O)Nc1ccnc(C(F)(F)F)c1]

### Step 1: Synthesis of 3-bromo-1-methyl-4-(trifluoromethyl)-N-(2-(trifluoromethyl)pyridin-4-yl)-1H-pyrazole-5-carboxamide (INT 4)

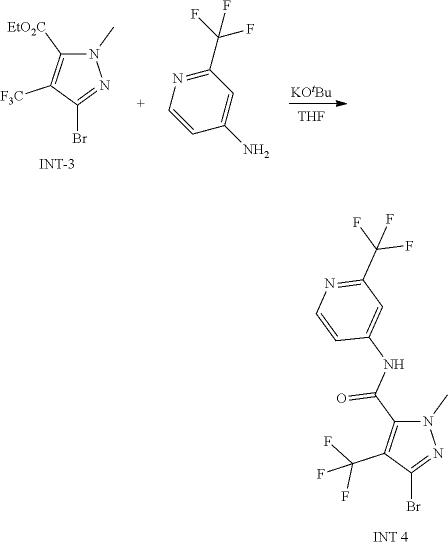

1 M KO^{t}Bu in THF (13 mL, 13.3 mmol, 2.00 eq) was added dropwise to a stirred solution of ethyl 5-bromo-2-methyl-4-(trifluoromethyl) pyrazole-3-carboxylate (2.0 g, 6.64 mmol, 1.00 eq) and 4-amino-2-(trifluoromethyl)pyridine (1.1 g, 6.64 mmol, 1.00 eq) in THF (60 mL) and the reaction was stirred at rt for 1 hour. The reaction was diluted with EtOAc and washed with water and brine. The combined organic phases were dried (MgSO₄) and concentrated under reduced pressure. The crude residue was purified by column chromatography eluting with 0-50% EtOAc in cyclohexane to give the title compound (2.4 g, 5.75 mmol, 86%) as an off-white solid. (ES, m/z): [M+H]⁺ 417.3 and 419.3, ¹H NMR (400 MHz, DMSO) δ 11.89 (s, 1H), 8.74 (d, J=5.5 Hz, 1H), 8.14 (d, J=1.8 Hz, 1H), 7.83 (dd, J=1.8, 5.5 Hz, 1H), 3.94 (s, 3H).

### Step 2: Synthesis of 3-(4,4,5,5-tetramethyl-1,3,2-dioxaborolan-2-yl) thieno[2,3-c]pyridine (INT 5)

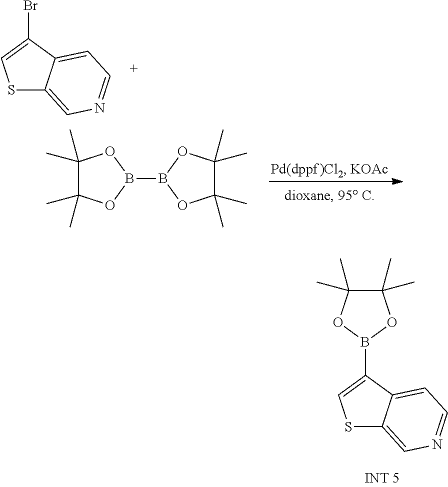

Pd(dppf)Cl₂ (121 mg, 0.163 mmol, 0.1 eq) was added to a stirred degassed mixture of bis(pinacolato)diboron (457 mg, 1.80 mmol, 1.10 eq), 3-bromothieno[2,3-c]pyridine (350 mg, 1.63 mmol, 1.00 eq) and potassium acetate (481 mg, 4.90 mmol, 3.00 eq) in 1,4-dioxane (10 mL), the reaction was then stirred at 100° C. and stirred for 16 h. The reaction mixture was cooled to rt and diluted with EtOAc. The organic layer was washed with NaHCO₃(aq. sat.), then brine, then dried and concentrated. The crude residue was purified by column chromatography eluting with 0-100% EtOAc in cyclohexane to give the title compound t with pinacol impurities. The residue was triturated with iPr₂O to give the title compound as a white solid (120 mg, 0.46 mmol, 28%). ¹H NMR (400 MHz, CDCl₃) δ 9.17 (s, 1H), 8.53 (d, J=5.4 Hz, 1H), 8.29 (s, 1H), 8.22 (d, J=5.5 Hz, 1H), 1.39 (s, 12H).

### Step 3: Synthesis of 1-methyl-3-(thieno[2,3-c]pyridin-3-yl)-4-(trifluoromethyl)-N-(2-(trifluoromethyl)pyridin-4-yl)-1H-pyrazole-5-carboxamide (I-1)

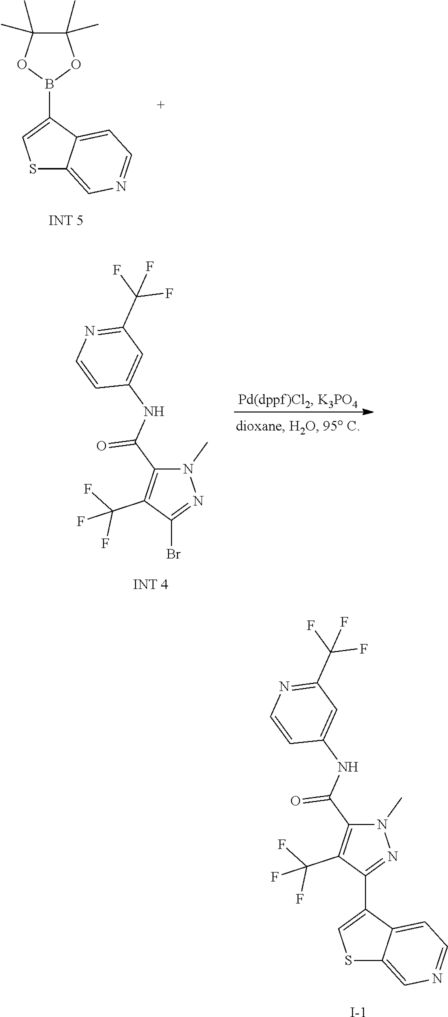 [SMILES: CC1(C)OB(c2csc3cnccc23)OC1(C)C ; Cn1nc(Br)c(C(F)(F)F)c1C(=O)Nc1ccnc(C(F)(F)F)c1 ; "INT 4" ; Cn1nc(-c2csc3cnccc23)c(C(F)(F)F)c1C(=O)Nc1ccnc(C(F)(F)F)c1 ; [I-]]

Pd(dppf)Cl₂ (11 mg, 0.0144 mmol, 0.1000 eq) was added to a stirred, degassed mixture of 3-bromo-1-methyl-4-(trifluoromethyl)-N-(2-(trifluoromethyl)pyridin-4-yl)-1H-pyrazole-5-carboxamide (60 mg, 0.144 mmol, 1.00 eq), 3 3-(4,4,5,5-tetramethyl-1,3,2-dioxaborolan-2-yl) thieno[2,3-c]pyridine (56 mg, 0.216 mmol, 1.50 eq) and K₃PO₄·H₂O (99 mg, 0.432 mmol, 3.00 eq) in 1,4-dioxane (3 mL) and water (0.3 mL). The reaction mixture was then stirred at 95° C. for 16 h. The reaction was cooled to rt and suspended between EtOAc and sat. aqueous NaHCO₃ solution. The layers were separated, and the organic layer was further washed with sat. aqueous NaHCO₃ solution and brine. The combined organic layers were dried and concentrated. The crude residue was purified by reverse phase preparative HPLC to give the title compound as an off-white solid (8.36 mg, 0.018 mmol, 12%). (ES, m/z): [M+H]⁺ 472.2 ¹H NMR (400 MHz, DMSO) δ 11.95 (s, 1H), 9.37 (s, 1H), 8.75 (d, J=5.1 Hz, 1H), 8.57 (d, J=5.6 Hz, 1H), 8.33 (s, 1H), 8.19 (s, 1H), 8.04 (d, J=5.8 Hz, 1H), 7.88 (d, J=5.1 Hz, 1H), 4.07 (s, 3H).

### Example 3—Synthesis of Additional Compounds

Compounds in Table 4 were prepared using a procedure analogous to that described in Example 2. Compound I-40 was obtained as a mixture of stereoisomers.

TABLE 4

| Compound | | Starting | |
|---|---|---|---|
| No. | Structure | Material | Analytical Data |
| I-2 | 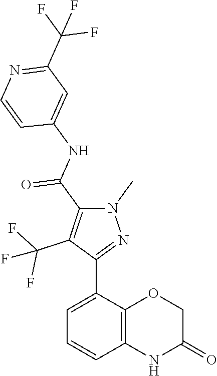 [SMILES: Cn1nc(-c2cccc3c2OCC(=O)N3)c(C(F)(F)F)c1C(=O)Nc1ccnc(C(F)(F)F)c1] | 8-(4,4,5,5- tetramethyl- 1,3,2- dioxaborolan-2- yl)-2H- benzo[b][1,4]ox- azin-3(4H)-one | (ES, m/z): [M + H]⁺ 486.2, ¹H NMR (400 MHz, DMSO) δ 11.91 (s, 1H), 10.87 (s, 1H), 8.75 (d, J = 5.5 Hz, 1H), 8.20 (d, J = 1.8 Hz, 1H), 7.90 (dd, J = 1.8, 5.4 Hz, IH), 7.06-7.01 (m, 2H), 6.96 (dd, J = 2.3, 6.8 Hz, 1H), 4.53 (s, 2H), 3.99 (s, 3H) |
| I-3 | 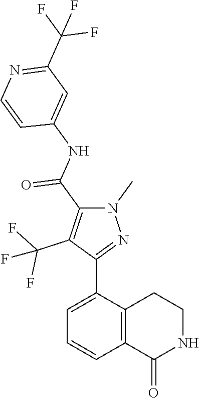 [SMILES: Cn1nc(-c2cccc3c2CCNC3=O)c(C(F)(F)F)c1C(=O)Nc1ccnc(C(F)(F)F)c1] | 5-(4,4,5,5- tetramethyl- 1,3,2- dioxaborolan-2- yl)-3,4- dihydroisoquino- lin-1(2H)-one | (ES, m/z): [M + H]⁺ 484.2, ¹H NMR (400 MHz, DMSO) δ 11.88 (br s, 1H), 8.75 (d, J = 5.6 Hz, 1H), 8.19 (d, J = 1.5 Hz, 1H), 8.09 (br s, 1H), 8.00 (dd, J = 2.8, 6.3 Hz, 1H), 7.89 (dd, J = 1.5, 5.6 Hz, 1H), 7.49-7.46 (m, 2H), 4.01 (s, 3H), 3.34- 3.32 (m, 2H), 2.74 (t, J = 6.5 Hz, 2H) |
| I--4 | 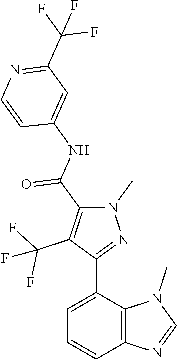 [SMILES: Cn1nc(-c2cccc3ncn(C)c23)c(C(F)(F)F)c1C(=O)Nc1ccnc(C(F)(F)F)c1] | 1-methyl-7- (4,4,5,5- tetramethyl- 1,3,2- dioxaborolan-2- yl)-1H- benzo[d]imidazole | (ES, m/z): [M + H]⁺ 469.2, ¹H NMR (400 MHz, DMSO) δ 11.85 (s, 1H), 8.77 (d, J = 5.6 Hz, 1H), 8.21-8.19 (m, 2H), 7.93-7.91 (m, 1H), 7.79 (d, J = 8.5 Hz, 1H), 7.30 (d, J = 8.0 Hz, 1H), 7.19 (d, J = 7.1 Hz, 1H), 4.05 (s,3H), 3.46 (s, 3H) |
| I-5 | 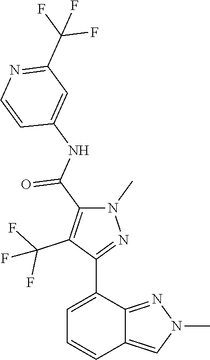 [SMILES: Cn1cc2cccc(-c3nn(C)c(C(=O)Nc4ccnc(C(F)(F)F)c4)c3C(F)(F)F)c2n1] | (2-methyl-2H- indazol-7- yl)boronic acid | (ES, m/z): [M + H]⁺ 469.2, ¹H NMR (400 MHz, DMSO) δ 11.96 (s, 1H), 8.75 (d, J = 5.3 Hz, 1H), 8.42 (s, 1H), 8.21 (d, J = 1.5 Hz, 1H), 7.92 (dd, J = 0.9, 4.2 Hz, 1H), 7.81 (d, J = 8.3 Hz, 1H), 7.23 (d, J = 6.6 Hz, 1H), 7.15-7.10 (m, 1H), 4.16 (s, 3H), 4.01 (s, 3H) |
| I-6 | 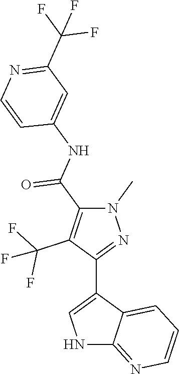 [SMILES: Cn1nc(-c2c[nH]c3ncccc23)c(C(F)(F)F)c1C(=O)Nc1ccnc(C(F)(F)F)c1] | tert-butyl 3- (4,4,5,5- tetramethyl- 1,3,2- dioxaborolan-2- yl)-1H- pyrrolo[2,3- b]pyridine-1- carboxylate | (ES, m/z): [M + H]⁺ 455.2, ¹H NMR (400 MHz, DMSO) δ 12.10 (s, 1H), 11.92 (s, 1H), 8.74 (d, J = 4.8 Hz, IH), 8.42 (dd, J = 1.4, 8.0 Hz, 1H), 8.33 (dd, J = 1.5, 4.5 Hz, 1H), 8.18 (s, 1H), 7.88 (d, J = 4.0 Hz, 1H), 7.68 (d, J = 1.8 Hz, 1H), 7.21 (dd, J = 4.7, 8.0 Hz, 1H), 4.00 (s, 3H) |
| I-7 | 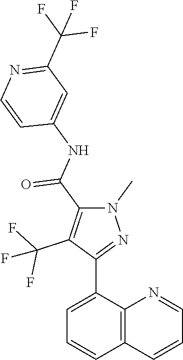 [SMILES: Cn1nc(-c2cccc3cccnc23)c(C(F)(F)F)c1C(=O)Nc1ccnc(C(F)(F)F)c1] | quinolin-8- ylboronic acid | (ES, m/z): [M + H]⁺ 466.2, ¹H NMR (400 MHz, DMSO) δ ppm 12.02-11.93 (m, 1H), 8.88 (dd, J = 1.8, 4.0 Hz, 1H), 8.74 (d, J = 5.1 Hz, 1H), 8.46 (dd, J = 1.5, 8.3 Hz, 1H), 8.22 (s, 1H), 8.14 (dd, J = 1.3, 8.1 Hz, 1H), 7.93 (d, J = 3.5 Hz, 1H), 7.78 (dd, J = 1.5, 7.1 Hz, 1H), 7.73-7.68 (m, 1H), 7.60 (dd, J = 4.3, 8.3 Hz, 1H), 4.04 (s, 3H) |
| I-8 | 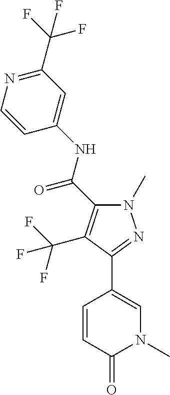 [SMILES: Cn1nc(-c2ccc(=O)n(C)c2)c(C(F)(F)F)c1C(=O)Nc1ccnc(C(F)(F)F)c1] | 1-methyl-5- (4,4,5,5- tetramethyl- 1,3,2- dioxaborolan-2- yl)pyridin- 2(1H)-one | (ES, m/z): [M + H]⁺ 446.2, ¹H NMR (400 MHz, DMSO) δ ppm 11.90 (s, 1H), 8.74-8.71 (m, 1H), 8.17-8.16 (m, 1H), 7.95 (d, J = 2.5 Hz, 1H), 7.85- 7.82 (m, 1H), 7.56 (dd, J = 2.5, 9.3 Hz, 1H), 6.54-6.51 (m, 1H), 3.96 (s, 3H), 3.52 (s, 3H) |
| I-9 | 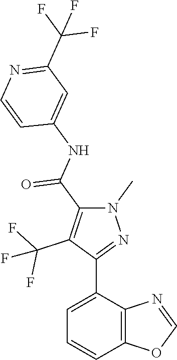 [SMILES: Cn1nc(-c2cccc3ocnc23)c(C(F)(F)F)c1C(=O)Nc1ccnc(C(F)(F)F)c1] | benzo[d]oxazol- 4-ylboronic acid | (ES, m/z): [M + H]⁺ 456.2, ¹H NMR (400 MHz, DMSO) δ 11.97 (s, 1H), 8.80 (s, 1H), 8.75 (d, J = 5.6 Hz, 1H), 8.21 (d, J = 1.5 Hz, 1H), 7.93-7.90 (m, 2H), 7.56 (dd, J = 8.0, 8.0 Hz, 1H), 7.46 (d, J = 7.1 Hz, 1H), 4.04 (s, 3H) |
| I-10 | 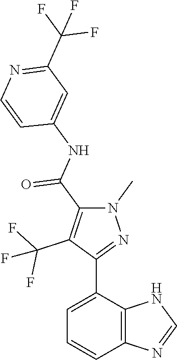 [SMILES: Cn1nc(-c2cccc3nc[nH]c23)c(C(F)(F)F)c1C(=O)Nc1ccnc(C(F)(F)F)c1] | (1H- benzo[d]imidazol- 7-yl)boronic acid | (ES, m/z): [M + H]⁺ 455.2, ¹H NMR (400 MHz, DMSO) δ ppm 12.47 (d, J = 101.3 Hz, 1H), 11.99 (s, 1H), 8.74 (d, J = 4.5 Hz, 1H), 8.25 (ddd, J = 8.7, 8.7, 8.7 Hz, 2H), 7.91-7.90 (m, 1H), 7.79-7.61 (m, 1H), 7.44- 7.19 (m, 2H), 4.04 (m, J = 18.7 Hz, 3H) |
| I-11 | 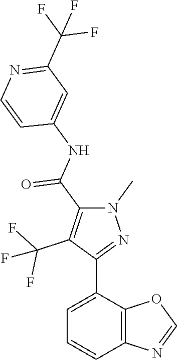 [SMILES: Cn1nc(-c2cccc3ncoc23)c(C(F)(F)F)c1C(=O)Nc1ccnc(C(F)(F)F)c1] | benzo[d]oxazol- 7-ylboronic acid | (ES, m/z): [M + H]⁺ 456.2, ¹H NMR (400 MHz, DMSO) δ ppm 11.97 (s, 1H), 8.84 (s, 1H), 8.75 (d, J = 5.3 Hz, 1H), 8.19 (s, 1H), 7.95 (dd, J = 3.3, 5.8 Hz, 1H), 7.89 (d, J = 4.0 Hz, 1H), 7.56-7.53 (m, 2H), 4.06 (s, 3H) |
| I-12 | 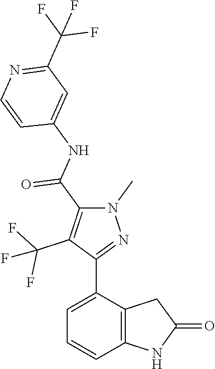 [SMILES: Cn1nc(-c2cccc3c2CC(=O)N3)c(C(F)(F)F)c1C(=O)Nc1ccnc(C(F)(F)F)c1] | 4-(4,4,5,5- tetramethyl- 1,3,2- dioxaborolan-2- yl)indolin-2-one | (ES, m/z): [M + H]⁺ 470.2, ¹H NMR (400 MHz, DMSO) δ ppm 11.92 (s, 1H), 10.55 (s, 1H), 8.74 (d, J = 5.1 Hz, 1H), 8.18 (s, 1H), 7.88 (d, J = 4.8 Hz, 1H), 7.32 (1H dd, J = 7.8, 7.8 Hz), 7.03 (1H, d, J = 7.9 Hz), 6.92 (1H, d, J = 7.7 Hz), 4.00 (3H, s), 3.54 (2H, s) |
| I-13 | 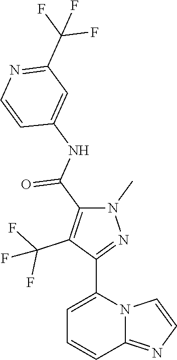 [SMILES: Cn1nc(-c2cccc3nccn23)c(C(F)(F)F)c1C(=O)Nc1ccnc(C(F)(F)F)c1] | imidazo[1,2- a]pyridin-5- ylboronic acid | (ES, m/z): [M + H]⁺ 455.2, ¹H NMR (400 MHz, DMSO) δ ppm 12.03-12.02 (m, 1H), 8.74-8.72 (m, 1H), 8.18 (s, 1H), 7.89 (s, 1H), 7.85 (s, 1H), 7.74 (d, J = 9.0 Hz, 1H), 7.67 (d, J = 1.1 Hz, 1H), 7.40 (dd, J = 6.9, 9.2 Hz, 1H), 7.05 (d, J = 6.8 Hz, 1H), 4.09 (s, 3H) |
| I-14 | 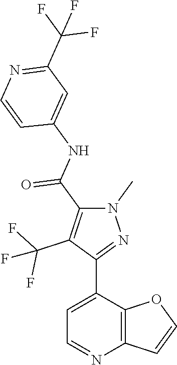 [SMILES: Cn1nc(-c2ccnc3ccoc23)c(C(F)(F)F)c1C(=O)Nc1ccnc(C(F)(F)F)c1] | furo[3,2- b]pyridin-7- ylboronic acid | (ES, m/z): [M + H]⁺ 456.2, ¹H NMR (400 MHz, DMSO) δ ppm 12.00 (s, 1H), 8.75 (d, J = 4.6 Hz, 1H), 8.66 (d, J = 4.9 Hz, 1H), 8.41 (d, J = 2.4 Hz, 1H), 8.19 (s, 1H), 7.88 (d, J = 4.1 Hz, 1H), 7.43 (d, J = 4.9 Hz, 1H), 7.27 (d, J = 2.3 Hz, 1H), 4.08 (s, 3H) |
| I-15 | 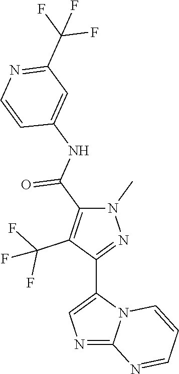 [SMILES: Cn1nc(-c2cnc3ncccn23)c(C(F)(F)F)c1C(=O)Nc1ccnc(C(F)(F)F)c1] | 3-(4,4,5,5- tetramcthyl- 1,3,2- dioxaborolan-2- yl)imidazo[1,2- ca]pyrimidine | (ES, m/z): [M + H]⁺ 456.2, ¹H NMR (400 MHz, DMSO) δ ppm 11.99 (s, 1H), 9.52 (dd, J = 1.9, 6.9 Hz, 1H), 8.75-8.70 (m, 2H), 8.17 (s, 1H), 8.06 (s, 1H), 7.88-7.83 (m, 1H), 7.29 (dd, J = 4.2, 6.9 Hz, 1H), 4.06 (s, 3H) |
| I-16 | 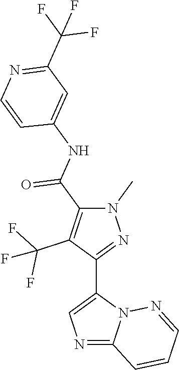 [SMILES: Cn1nc(-c2cnc3cccnn23)c(C(F)(F)F)c1C(=O)Nc1ccnc(C(F)(F)F)c1] | imidazo[1,2- b]pyridazin-3- ylboronic acid | (ES, m/z): [M + H]⁺ 456.2, ¹H NMR (400 MHz, DMSO) δ ppm 11.99-11.99 (m, 1H), 8.74 (d, J = 3.6 Hz, 1H), 8.59 (dd, J = 1.5, 4.4 Hz, 1H), 8.26 (dd, J = 1.6, 9.3 Hz, 1H), 8.20 (s, 1H), 8.00 (s, 1H), 7.87-7.86 (m, 1H), 7.36 (dd, J = 4.5, 9.2 Hz, 1H), 4.06 (s, 3H) |
| I-17 | 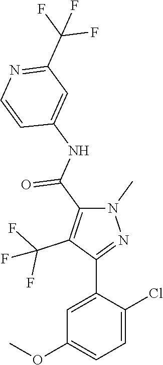 [SMILES: COc1ccc(Cl)c(-c2nn(C)c(C(=O)Nc3ccnc(C(F)(F)F)c3)c2C(F)(F)F)c1] | (2-chloro-5- methoxyphenyl) boronic acid | (ES, m/z): [M + H]⁺ 479.2, ¹H NMR (400 MHz, DMSO) δ ppm 11.89-11.87 (m, 1H), 8.74 (d, J = 5.4 Hz, 1H), 8.18 (s, 1H), 7.90 (d, J = 4.9 Hz, 1H), 7.51 (d, J = 8.8 Hz, 1H), 7.11 (dd, J = 3.1, 8.9 Hz, 1H), 6.93 (d, J = 3.0 Hz, 1H), 4.02 (s, 3H), 3.79 (s, 3H) |
| I-18 | 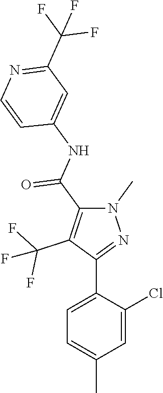 [SMILES: Cc1ccc(-c2nn(C)c(C(=O)Nc3ccnc(C(F)(F)F)c3)c2C(F)(F)F)c(Cl)c1] | (2-chloro-4- mcthylphcnyl)boro- nic acid | (ES, m/z): [M + H]⁺ 463.2, ¹H NMR (400 MHz, DMSO) δ ppm 11.89 (s, 1H), 8.74 (d, J = 5.5 Hz, 1H), 8.18 (s, 1H), 7.90 (d, J = 4.8 Hz, 1H), 7.45 (s, 1H), 7.28 (d, J = 4.3 Hz, 2H), 4.01 (s, 3H), 2.38 (s, 3H) |
| I-19 | 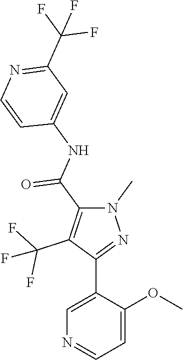 [SMILES: COc1ccncc1-c1nn(C)c(C(=O)Nc2ccnc(C(F)(F)F)c2)c1C(F)(F)F] | (4- methoxypyridin- 3-yl)boronic acid | (ES, m/z): [M + H]⁺ 446.2, ¹H NMR (400 MHz, DMSO) δ ppm 11.91 (s, 1H), 8.72-8.70 (m, 1H), 8.56 (d, J = 5.8 Hz, 1H), 8.34 (s, 1H), 8.17 (s, 1H), 7.89- 7.85 (m, 1H), 7.22 (d, J = 5.8 Hz, 1H), 4.01 (s, 3H), 3.86 (s, 3H) |
| I-20 | 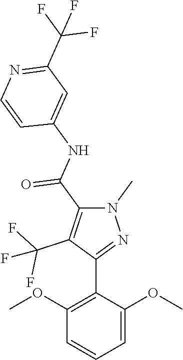 [SMILES: COc1cccc(OC)c1-c1nn(C)c(C(=O)Nc2ccnc(C(F)(F)F)c2)c1C(F)(F)F] | (2,6- dimethoxyphenyl) boronic acid | (ES, m/z): [M + H]⁺ 475.2, ¹H NMR (400 MHz, DMSO) δ ppm 11.88 (s, 1H), 8.71 (s, 1H), 8.18 (s, 1H), 7.92 (s, 1H), 7.39 (dd, J = 8.3, 8.3 Hz, 1H), 6.74 (d, J = 8.5 Hz, 2H), 3.96 (s, 3H), 3.69 (s, 6H), |
| I-21 | 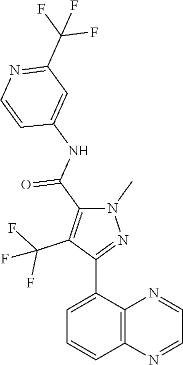 [SMILES: Cn1nc(-c2cccc3nccnc23)c(C(F)(F)F)c1C(=O)Nc1ccnc(C(F)(F)F)c1] | quinoxalin-5- ylboronic acid | (ES, m/z): [M + H]⁺ 467.2, ¹H NMR (400 MHz, DMSO) δ 12.00 (s, 1H), 9.03 (d, J = 1.8 Hz, 1H), 8.95 (d, J = 1.8 Hz, 1H), 8.75 (d, J = 5.4 Hz, 1H), 8.26 (dd, J = 1.5, 8.4 Hz, 1H), 8.22 (s, IH), 8.00-7.96 (m, 1H), 7.92 (dd, J = 1.4, 7.1 Hz, 2H), 4.05 (s, 3H) |
| I-22 | 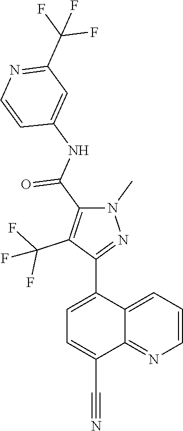 [SMILES: Cn1nc(-c2ccc(C#N)c3ncccc23)c(C(F)(F)F)c1C(=O)Nc1ccnc(C(F)(F)F)c1] | (8- cyanoquinolin- 5-yl)boronic acid | (ES, m/z): [M + H]⁺ 491.2, ¹H NMR (400 MHz, DMSO) δ ppm 11.98 (s, 1H), 9.18 (dd, J = 1.6, 4.2 Hz, 1H), 8.76-8.71 (m, 1H), 8.51 (d, J = 7.3 Hz, 1H), 8.34 (dd, J = 1.5, 8.6 Hz, 1H), 8.20-8.17 (m, 1H), 7.87-7.86 (m, 1H), 7.83-7.79 (m, 2H), 4.09 (s, 3H) |
| I-23 | 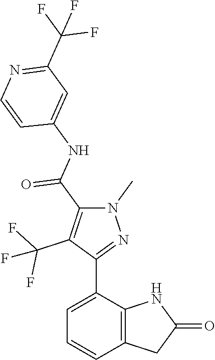 [SMILES: Cn1nc(-c2cccc3c2NC(=O)C3)c(C(F)(F)F)c1C(=O)Nc1ccnc(C(F)(F)F)c1] | 7-(4,4,5,5- tetramethyl- 1,3,2- dioxaborolan-2- yl)indolin-2-one | (ES, m/z): [M + H]⁺ 470.2, ¹H NMR (400 MHz, DMSO) δ 11.89 (s, 1H), 10.17 (s, 1H), 8.75 (d, J = 5.3 Hz, 1H), 8.19 (s, 1H), 7.90 (d, J = 4.5 Hz, 1H), 7.31 (d, J = 7.1 Hz, 1H), 7.24 (d, J = 7.8 Hz, 1H), 7.07 (dd, J = 7.6, 7.6 Hz, 1H), 3.98 (s, 3H), 3.59 (s, 2H) |
| I-24 | 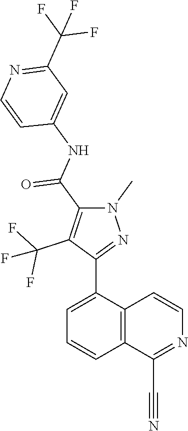 [SMILES: Cn1nc(-c2cccc3c(C#N)nccc23)c(C(F)(F)F)c1C(=O)Nc1ccnc(C(F)(F)F)c1] | 5-(4,4,5,5- tetramethyl- 1,3,2- dioxaborolan-2- yl)isoquinoline- 1-carbonitrile | (ES, m/z): [M + H]⁺ 491.2, ¹H NMR (400 MHz, DMSO) δ 11.98 (s, 1H), 8.78 (d, J = 5.8 Hz, 1H), 8.75 (s, 1H), 8.44 (d, J = 8.3 Hz, 1H), 8.20 (s, 1H), 8.11-8.06 (m, 1H), 8.04-8.00 (m, 2H), 7.89-7.88 (m, 1H), 4.09 (s, 3H) |
| I-25 | 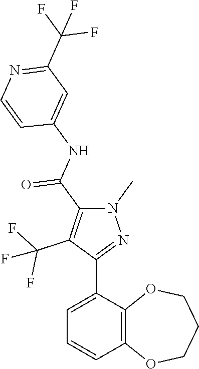 [SMILES: Cn1nc(-c2cccc3c2OCCCO3)c(C(F)(F)F)c1C(=O)Nc1ccnc(C(F)(F)F)c1] | 2-(3,4-dihydro- 2H- benzo[b][1,4]di- oxepin-6-yl)- 4,4,5,5- tetramethyl- 1,3,2- dioxaborolane | (ES, m/z): [M + H]⁺ 487.2, ¹H NMR (400 MHz, DMSO) δ 11.88 (s, 1H), 8.74 (d, J = 5.5 Hz, 1H), 8.20 (s, 1H), 7.90 (d, J = 4.5 Hz, 1H), 7.08 (dd, J = 1.8, 7.9 Hz, 1H), 7.00 (dd, J = 7.7, 7.7 Hz, 1H), 6.94 (dd, J = 1.8, 7.5 Hz, 1H), 4.18 (t, J = 5.8 Hz, 2H), 4.11 (t, J = 5.6 Hz, 2H), 3.98 (s, 3H), 2.15-2.07 (m, 2H) |
| I-26 | 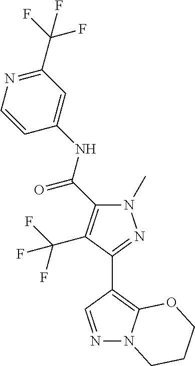 [SMILES: Cn1nc(-c2cnn3c2OCCC3)c(C(F)(F)F)c1C(=O)Nc1ccnc(C(F)(F)F)c1] | 3-(4,4,5,5- tetramcthyl- 1,3,2- dioxaborolan-2- yl)-6,7-dihydro- 5H- pyrazolo[5,1- b][1,3]oxazine | (ES, m/z): [M + H]⁺ 461.5, ¹H NMR (400 MHz, DMSO) δ 11.81 (s, 1H), 8.73 (d, J = 5.3 Hz, 1H), 8.16 (d, J = 1.5 Hz, 1H), 7.85 (d, J = 4.5 Hz, 1H), 7.40 (s, 1H), 4.34 (dd, J = 5.2, 5.2 Hz, 2H), 4.15 (dd, J = 6.2, 6.2 Hz, 2H), 3.90 (s, 3H), 2.25- 2.18 (m, 2H) |
| I-27 | 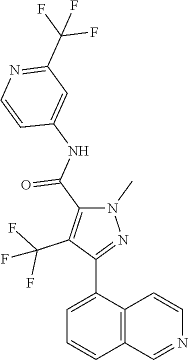 [SMILES: Cn1nc(-c2cccc3cnccc23)c(C(F)(F)F)c1C(=O)Nc1ccnc(C(F)(F)F)c1] | isoquinolin-5- ylboronic acid | (ES, m/z): [M + H]⁺ 466.271 ¹H NMR (400 MHz, DMSO) δ 11.96 (br s, 1H), 9.43 (s, 1H), 8.76 (d, J = 5.4 Hz, 1H), 8.57 (d, J = 6.0 Hz, 1H), 8.33-8.27 (m, 1H), 8.21 (d, J = 1.5 Hz, 1H), 7.90 (d, J = 4.2 Hz, 1H), 7.84- 7.80 (m, 2H), 7.64 (d, J = 6.0 Hz, 1H), 4.08 (s, 3H) |
| I-28 | 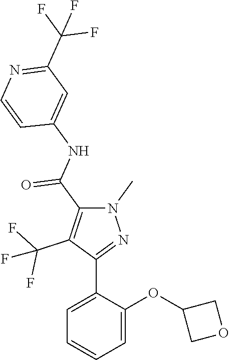 [SMILES: Cn1nc(-c2ccccc2OC2COC2)c(C(F)(F)F)c1C(=O)Nc1ccnc(C(F)(F)F)c1] | 4,4,5,5- tetramethyl-2- (2-(oxetan-3- yloxy)phenyl)- 1,3,2- dioxaborolane | (ES, m/z): [M + H]⁺ 487.179 ¹H NMR (400 MHz, DMSO) δ 11.84 (s, 1H), 8.74 (d, J = 5.5 Hz, 1H), 8.20 (d, J = 1.5 Hz, 1H), 7.92-7.84 (m, 1H), 7.41 (dt, J = 7.7, 1.8 Hz, 1H), 7.34 (dd, J = 7.5, 1.6 Hz, 1H), 7.07 (t, J = 7.5 Hz, 1H), 6.72 (d, J = 8.2 Hz, 1H), 5.30 (p, J = 5.4 Hz, 1H), 4.92 (t, J = 6.7 Hz, 2H), 4.50 (dd, J = 7.0, 5.4 Hz, 2H), 4.01 (s, 3H) |
| I-29 | 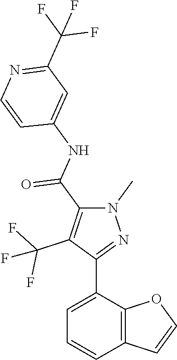 [SMILES: Cn1nc(-c2cccc3ccoc23)c(C(F)(F)F)c1C(=O)Nc1ccnc(C(F)(F)F)c1] | benzofuran-7- ylboronic acid | (ES, m/z): [M + H]⁺ 455.25 ¹H NMR (400 MHz, DMSO) δ 11.96 (br s, 1H), 8.74 (d, J = 5.4 Hz, 1H), 8.20 (d, J = 1.6 Hz, 1H), 8.04 (d, J = 2.2 Hz, 1H), 7.89 (d, J = 4.3 Hz, 1H), 7.79 (dd, J = 5.2, 3.8 Hz, 1H), 7.40- 7.33 (m, 2H), 7.06 (d, J = 2.2 Hz, 1H), 4.04 (s, 3H) |
| I-30 | 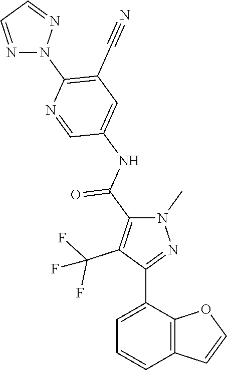 [SMILES: Cn1nc(-c2cccc3ccoc23)c(C(F)(F)F)c1C(=O)Nc1cnc(-n2nccn2)c(C#N)c1] | benzofuran-7- ylboronic acid | (ES, m/z): [M + H]⁺ 479.2 ¹H NMR (400 MHz, DMSO) δ 12.00 (br s, 1H), 9.04 (br s, 1H), 8.84 (d, J = 2.5 Hz, 1H), 8.32 (s, 2H), 8.05 (d, J = 2.2 Hz, 1H), 7.80 (t, J = 4.2 Hz, 1H), 7.38 (d, J = 4.8 Hz, 2H), 7.07 (d, J = 2.2 Hz, 1H), 4.07 (s, 3H) |
| I-31 | 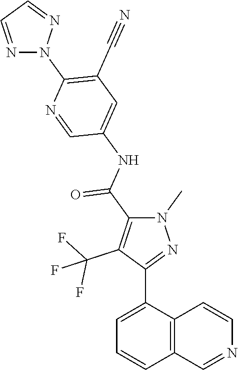 [SMILES: Cn1nc(-c2cccc3cnccc23)c(C(F)(F)F)c1C(=O)Nc1cnc(-n2nccn2)c(C#N)c1] | isoquinolin-5- ylboronic acid | (ES, m/z): [M + H]⁺ 490.2 ¹H NMR (400 MHz, DMSO) δ 12.04 (br s, 1H), 9.43 (s, 1H), 9.01 (br s, 1H), 8.87 (d, J = 2.4 Hz, 1H), 8.57 (d, J = 5.9 Hz, 1H), 8.34-8.26 (m, 3H), 7.86- 7.79 (m, 2H), 7.64 (d, J = 5.9 Hz, 1H), 4.10 (s, 3H) |
| I-32 | 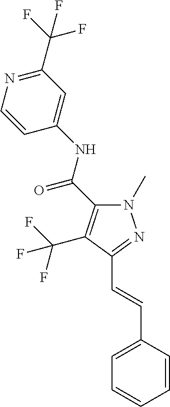 [SMILES: Cn1nc(/C=C/c2ccccc2)c(C(F)(F)F)c1C(=O)Nc1ccnc(C(F)(F)F)c1] | (E)- styrylboronic acid | (ES, m/z): [M + H]⁺ 441.2 ¹H NMR (400 MHz, DMSO) δ 11.83 (s, 1H), 8.74 (d, J = 5.3 Hz, 1H), 8.16 (s, 1H), 7.86 (d, J = 5.0 Hz, 1H), 7.64 (d, J = 7.4 Hz, 2H), 7.56-7.30 (m, 4H), 7.06 (d, J = 16.4 Hz, 1H), 3.97 (s, 3H) |
| I-33 | 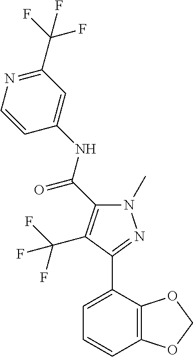 [SMILES: Cn1nc(-c2cccc3c2OCO3)c(C(F)(F)F)c1C(=O)Nc1ccnc(C(F)(F)F)c1] | benzo[d][1,3]di- oxol-4- ylboronic acid | (ES, m/z): [M + H]⁺ 459.2 ¹H NMR (400 MHz, DMSO) δ 11.93 (br s, 1H), 8.71 (s, 1H), 8.16 (s, 1H), 7.84 (s, 1H), 7.04 (dd, J = 7.6, 1.3 Hz, 1H), 6.95 (t, J = 7.7 Hz, 1H), 6.89 (dd, J = 7.9, 1.3 Hz, 1H), 6.06 (s, 2H), 3.99 (s, 3H) |
| I-34 | 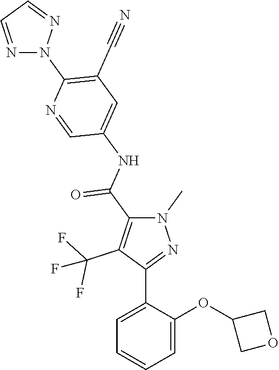 [SMILES: Cn1nc(-c2ccccc2OC2COC2)c(C(F)(F)F)c1C(=O)Nc1cnc(-n2nccn2)c(C#N)c1] | 4,4,5,5- tetramethyl-2- (2-(oxetan-3- yloxy)phenyl)- 1,3,2- dioxaborolane | (ES, m/z): [M + H]⁺ 511.2 ¹H NMR (400 MHz, DMSO) δ 11.89 (br s, 1H), 9.02 (br s, 1H), 8.83 (d, J = 2.4 Hz, 1H), 8.31 (s, 2H), 7.41 (dt, J = 7.4, 1.6 Hz, 1H), 7.35 (dd, J = 7.5, 1.6 Hz, 1H), 7.08 (t, J = 7.5 Hz, 1H), 6.72 (d, J = 8.3 Hz, 1H), 5.31 (p, J = 5.5 Hz, 1H), 4.92 (t, J = 6.7 Hz, 2H), 4.51 (dd, J = 6.9, 5.4 Hz, 2H), 4.04 (s, 3H) |
| I-35 | 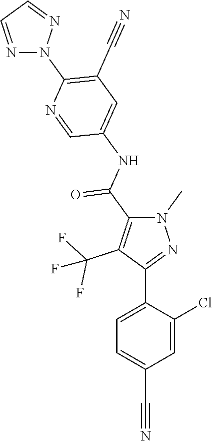 [SMILES: Cn1nc(-c2ccc(C#N)cc2Cl)c(C(F)(F)F)c1C(=O)Nc1cnc(-n2nccn2)c(C#N)c1] | (2-chloro-4- cyanophenyl)bo- ronic acid | (ES, m/z): [M + H]⁺ 498.2 ¹H NMR (400 MHz, DMSO) δ 11.98 (br s, 1H), 8.99 (br s, 1H), 8.84 (d, J = 2.4 Hz, 1H), 8.31 (s, 2H), 8.27 (d, J = 1.4 Hz, 1H), 7.97 (dd, J = 7.9, 1.5 Hz, 1H), 7.66 (d, J = 7.9 Hz, 1H), 4.07 (s, 3H) |
| I-36 | 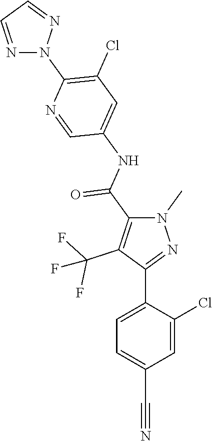 [SMILES: Cn1nc(-c2ccc(C#N)cc2Cl)c(C(F)(F)F)c1C(=O)Nc1cnc(-n2nccn2)c(Cl)c1] | (2-chloro-4- cyanophenyl)bo- ronic acid | (ES, m/z): [M + H]⁺ 507.2 ¹H NMR (400 MHz, DMSO) δ 11.93 (br s, 1H), 8.72 (br s, 1H), 8.63 (d, J = 2.1 Hz, 1H), 8.23 (s, 1H), 8.17 (s, 2H), 7.96 (dd, J = 8.0, 1.2 Hz, 1H), 7.65 (d, J = 7.9 Hz, 1H), 4.06 (s, 3H) |
| I-37 | 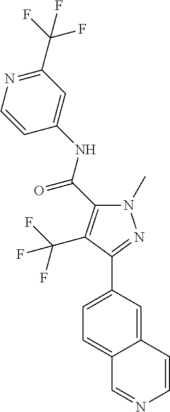 [SMILES: Cn1nc(-c2ccc3cnccc3c2)c(C(F)(F)F)c1C(=O)Nc1ccnc(C(F)(F)F)c1] | isoquinolin-6- ylboronic acid | (ES, m/z): [M + H]⁺ 466.2 ¹H NMR (400 MHz, DMSO) δ 11.99 (br s, 1H), 9.40 (s, 1H), 8.72 (br s, 1H), 8.58 (d, J = 5.8 Hz, 1H), 8.27 (d, J = 8.5 Hz, 1H), 8.21-8.14 (m, 2H), 7.97 (d, J = 5.8 Hz, 1H), 7.88 (d, J = 8.5 Hz, 1H), 7.85 (br s, 1H), 4.04 (s, 3H) |
| I-38 | 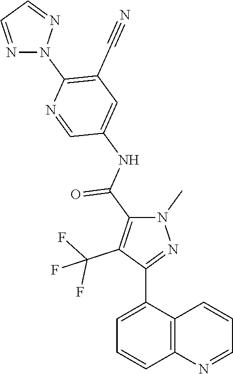 [SMILES: Cn1nc(-c2cccc3ncccc23)c(C(F)(F)F)c1C(=O)Nc1cnc(-n2nccn2)c(C#N)c1] | quinolin-5- ylboronic acid | (ES, m/z): [M + H]⁺ 490.2 ¹H NMR (400 MHz, DMSO) δ 12.02 (br s, 1H), 9.02 (br s, 1H), 8.99 (dd, J = 4.1, 1.6 Hz, 1H), 8.87 (d, J = 2.4 Hz, 1H), 8.32 (s, 2H), 8.18 (d, J = 8.44 Hz, 2H), 7.89 (dd, J = 8.3, 7.1 Hz, 1H), 7.66 (d, J = 7.1 Hz, 1H), 7.61 (dd, J = 8.5, 4.1 Hz, 1H), 4.10 (s, 3H) |
| I-39 | 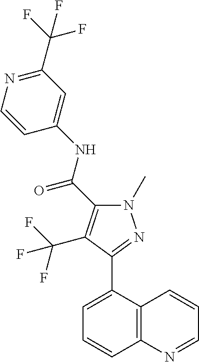 [SMILES: Cn1nc(-c2cccc3ncccc23)c(C(F)(F)F)c1C(=O)Nc1ccnc(C(F)(F)F)c1] | quinolin-5- ylboronic acid | (ES, m/z): [M + H]⁺ 466.2 ¹H NMR (400 MHz, DMSO) δ 11.98 (s, 1H), 8.98 (dd, J = 4.1, 1.5 Hz, 1H), 8.74 (d, J = 5.4 Hz, 1H), 8.27-8.14 (m, 3H), 7.95- 7.85 (m, 2H), 7.65 (d, J = 7.0 Hz, 1H), 7.60 (dd, J = 8.6, 4.1 Hz, 1H), 4.07 (s, 3H) |
| I-40 | 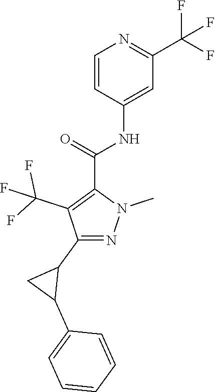 [SMILES: Cn1nc(C2CC2c2ccccc2)c(C(F)(F)F)c1C(=O)Nc1ccnc(C(F)(F)F)c1] | rac-((1R,2R)-2- phenylcyclopro- pyl)boronic acid | (ES, m/z): [M + H]⁺ 455.2 ¹H NMR (400 MHz, DMSO) δ 11.78 (s, 1H), 8.72 (d, J = 5.4 Hz, 1H), 8.15 (d, J = 1.4 Hz, 1H), 7.84 (d, J = 4.2 Hz, 1H), 7.34-7.25 (m, 2H), 7.23-7.15 (m, 3H), 3.87 (s, 3H), 2.37-2.29 (m, IH), 2.20-2.11 (m, 1H), 1.66-1.59 (m, 1H), 1.54-1.47 (m, 1H) |
| I-41 | 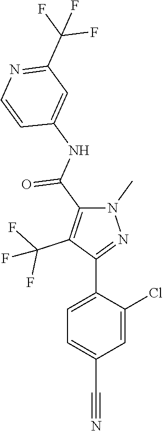 [SMILES: Cn1nc(-c2ccc(C#N)cc2Cl)c(C(F)(F)F)c1C(=O)Nc1ccnc(C(F)(F)F)c1] | (2-chloro-4- cyanophenyl)bo- ronic acid | (ES, m/z): [M + H]⁺ 474.2 ¹H NMR (400 MHz, DMSO) δ 11.90 (br s, 1H), 8.54 (br s, 1H), 8.18 (d, J = 0.9 Hz, 1H), 8.04 (br s, 1H), 7.88 (dd, J = 7.9, 1.5 Hz, 1H), 7.69 (br s, 1H), 7.57 (d, J = 7.9 Hz, 1H), 3.97 (s, 3H) |
| I-42 | 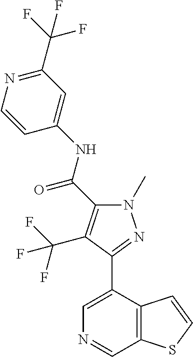 [SMILES: Cn1nc(-c2cncc3sccc23)c(C(F)(F)F)c1C(=O)Nc1ccnc(C(F)(F)F)c1] | 1-methyl-3- (thieno[2,3- c]pyridin-4-yl)- 4- (trifluoromethyl)- N-(2- (trifluoromethyl) pyridin-4-yl)- 1H-pyrazole-5- carboxamide | (ES, m/z): [M + H]⁺ 472 ¹H NMR (400 MHz, DMSO) δ 11.95 (s, 1H), 9.40 (s, 1H), 8.76 (d, J = 5.3 Hz, 1H), 8.50 (s, 1H), 8.25 (d, J = 5.4 Hz, 1H), 8.20 (s, 1H), 7.90 (d, J = 4.4 Hz, 1H), 7.55 (d, J = 5.4 Hz, 1H), 4.08 (s, 3H) |
| I-43 | 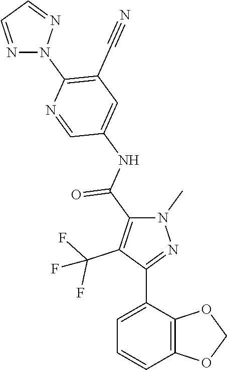 [SMILES: Cn1nc(-c2cccc3c2OCO3)c(C(F)(F)F)c1C(=O)Nc1cnc(-n2nccn2)c(C#N)c1] | benzo[d][1,3]di- oxol-4- ylboronic acid | (ES, m/z): [M + H]⁺ 483.2 ¹H NMR (400 MHz, DMSO) δ 11.98 (br s, 1H), 9.01 (s, 1H), 8.82 (d, J = 4.0 Hz, 1H), 8.31 (s, 2H), 7.04 (dd, J = 7.5, 1.4 Hz, 1H), 6.95 (t, J = 7.4 Hz, 1H), 6.91 (dd, J = 7.9, 1.2 Hz, 1H), 6.06 (s, 2H), 4.02 (s, 3H) |
| I-44 | 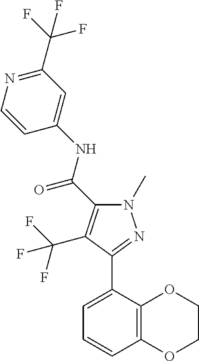 [SMILES: Cn1nc(-c2cccc3c2OCCO3)c(C(F)(F)F)c1C(=O)Nc1ccnc(C(F)(F)F)c1] | (2,3- dihydrobenzo[b] [1,4]dioxin-5- yl)boronic acid | (ES, m/z): [M + H]⁺ 473.2 ¹H NMR (400 MHz, DMSO) δ 11.88 (br s, 1H), 8.70 (br s, 1H), 8.17 (s, 1H), 7.85 (br s, 1H), 6.97 (dd, J = 8.0, 1.8 Hz, 1H), 6.90 (t, J = 7.7 Hz, 1H), 6.84 (dd, J = 7.6, 1.8 Hz, 1H), 4.28-4.23 (m, 2H), 4.23-4.17 (m, 2H), 3.97 (s, 3H) |
| I-45 | 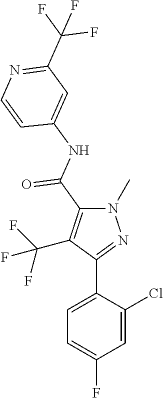 [SMILES: Cn1nc(-c2ccc(F)cc2Cl)c(C(F)(F)F)c1C(=O)Nc1ccnc(C(F)(F)F)c1] | (2-chloro-4- fluorophenyl)bo- ronic acid | (ES, m/z): [M + H]⁺ 467.2 ¹H NMR (400 MHz, DMSO) δ 11.90 (s, 1H), 8.75 (d, J = 5.3 Hz, 1H), 8.18 (s, 1H), 7.90 (d, J = 4.3 Hz, 1H), 7.65 (dd, J = 8.8, 2.5 Hz, 1H), 7.49 (dd, J = 8.6, 6.2 Hz, 1H), 7.36 (dt, J = 8.4, 2.6 Hz, 1H), 4.02 (s, 3H) |
| I-46 | 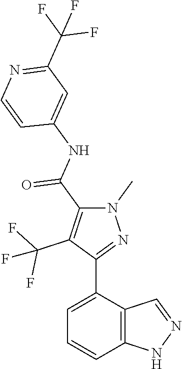 [SMILES: Cn1nc(-c2cccc3[nH]ncc23)c(C(F)(F)F)c1C(=O)Nc1ccnc(C(F)(F)F)c1] | 4-(4,4,5,5- tetramethyl- 1,3,2- dioxaborolan-2- yl)-1H-indazole | (ES, m/z): [M + H]⁺ 455.2 ¹H NMR (400 MHz, DMSO) δ 13.28 (s, 1H), 11.96 (br s, 1H), 8.74 (d, J = 4.4 Hz, 1H), 8.19 (s, 2H), 7.88 (d, J = 3.5 Hz, 1H), 7.65 (d, J = 8.3 Hz, 1H), 7.47 (dd, J = 8.3, 7.1 Hz, 1H), 7.27 (d, J = 7.1 Hz, 1H), 4.06 (s, 3H) |
| I-47 | 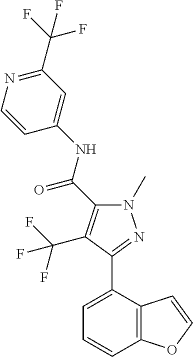 [SMILES: Cn1nc(-c2cccc3occc23)c(C(F)(F)F)c1C(=O)Nc1ccnc(C(F)(F)F)c1] | 2-(benzofuran- 4-yl)-4,4,5,5- tetramethyl- 1,3,2- dioxaborolane | (ES, m/z): [M + H]⁺ 455.2 ¹H NMR (400 MHz, DMSO) δ 11.94 (s, 1H), 8.74 (br s, 1H), 8.19 (s, 1H), 8.10 (d, J = 2.2 Hz, 1H), 7.88 (br s, 1H), 7.73 (d, J = 8.2 Hz, 1H), 7.46 (t, J = 8.0 Hz, 1H), 7.39 (d, J = 7.4 Hz, 1H), 7.05 (d, J = 1.4 Hz, 1H), 4.04 (s, 3H) |
| I-48 | 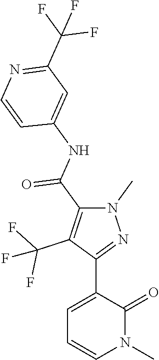 [SMILES: Cn1nc(-c2cccn(C)c2=O)c(C(F)(F)F)c1C(=O)Nc1ccnc(C(F)(F)F)c1] | (1-methyl-2- oxo-1,2- dihydropyridin- 3-yl)boronic acid | (ES, m/z): [M + H]⁺ 446.2 ¹H NMR (400 MHz, DMSO) δ 11.89 (br s, 1H), 8.71 (br s, 1H), 8.18 (s, 1H), 7.86 (br s, 1H), 7.83 (dd, J = 6.8, 1.9 Hz, 1H), 7.50 (dd, J = 6.9, 1.9 Hz, 1H), 6.30 (t, J = 6.8 Hz, 1H), 3.96 (s, 3H), 3.49 (s, 3H) |
| I-49 | 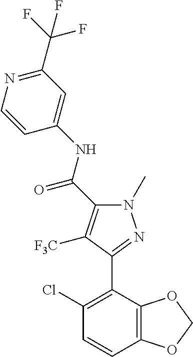 [SMILES: Cn1nc(-c2c(Cl)ccc3c2OCO3)c(C(F)(F)F)c1C(=O)Nc1ccnc(C(F)(F)F)c1] | (5- chlorobenzo[d] [1,3]dioxol-4- yl)boronic acid | (ES, m/z): [M + H]⁺ 493.2 ¹H NMR (400 MHz, DMSO) δ 11.97 (s, 1H), 8.80 (br s, 1H), 8.23 (s, 1H), 7.95 (br s, 1H), 7.12 (s, 2H), 6.16 (s, 2H), 4.08 (s, 3H) |
| I-50 | 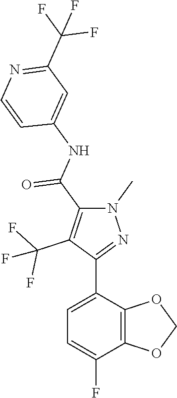 [SMILES: Cn1nc(-c2ccc(F)c3c2OCO3)c(C(F)(F)F)c1C(=O)Nc1ccnc(C(F)(F)F)c1] | (7- fluorobenzo[d][1,3] dioxol-4- yl)boronic acid | (ES, m/z): [M + H]⁺ 477.2 ¹H NMR (400 MHz, DMSO)δ 11.91 (br s, 1H), 8.72 (br s, 1H), 8.16 (s, 1H), 7.85 (br s, 1H), 7.02-6.90 (m, 2H), 6.18 (s, 2H), 3.99 (s, 3H) |
| I-51 | 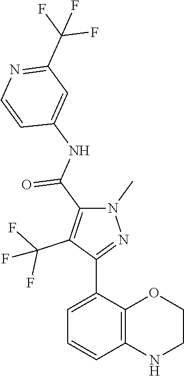 [SMILES: Cn1nc(-c2cccc3c2OCCN3)c(C(F)(F)F)c1C(=O)Nc1ccnc(C(F)(F)F)c1] | 8-(4,4,5,5- tetramethyl- 1,3,2- dioxaborolan-2- yl)-3,4-dihydro- 2H- benzo[b][1,4]oxa- zine | (ES, m/z): [M + H]⁺ 472.2 ¹H NMR (400 MHz, DMSO) δ 11.84 (br s, 1H), 8.73 (d, J = 4.9 Hz, 1H), 8.18 (d, J = 1.6 Hz, 1H), 7.88 (d, J = 4.9 Hz, 1H), 6.72 (t, J = 7.5 Hz, 1H), 6.64 (dd, J = 7.9, 1.6 Hz, 1H), 6.47 (dd, J = 7.4, 6.5 Hz, 1H), 5.91 (br s, 1H), 4.06 (t, J = 4.2 Hz, 2H), 3.95 (s, 3H), 3.34-3.26 (m, 2H, partly covered by residual water peak) |
| I-52 | 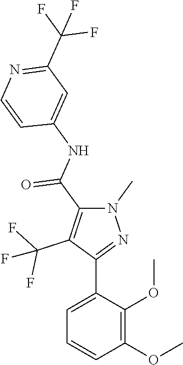 [SMILES: COc1cccc(-c2nn(C)c(C(=O)Nc3ccnc(C(F)(F)F)c3)c2C(F)(F)F)c1OC] | (2,3- dimethoxyphenyl) boronic acid | (ES, m/z): [M + H]⁺ 475.2 ¹H NMR (400 MHz, DMSO) δ 11.88 (s, 1H), 8.74 (br s, 1H), 8.18 (s, 1H), 7.89 (br s, 1H), 7.18 (dd, J = 8.3, 1.8 Hz, 1H), 7.14 (t, J = 7.8 Hz, 1H), 6.86 (dd, J = 7.4, 1.8 Hz, 1H), 4.00 (s, 3H), 3.86 (3H, s), 3.67 (s, 3H) |
| I-53 | 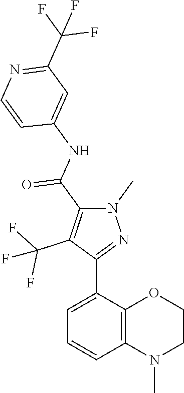 [SMILES: CN1CCOc2c(-c3nn(C)c(C(=O)Nc4ccnc(C(F)(F)F)c4)c3C(F)(F)F)cccc21] | 4-methyl-8- (4,4,5,5- tetramethyl- 1,3,2- dioxaborolan-2- yl)-3,4-dihydro- 2H- benzo[b][1,4]oxa- zine | (ES, m/z): [M + H]⁺ 486.2 ¹H NMR (400 MHz, DMSO) δ 11.86 (s, 1H), 8.74 (d, J = 5.4 Hz, 1H), 8.19 (d, J = 1.6 Hz, 1H), 7.89 (dd, J = 5.4, 1.7 Hz, 1H), 6.83 (t, J = 7.8 Hz, 1H), 6.78 (dd, J = 8.2, 1.9 Hz, 1H), 6.58 (dd, J = 7.2, 1.7 Hz, 1H), 4.18 (t, J = 4.5 Hz, 2H), 3.96 (s, 3H), 3.26 (t, J = 4.5 Hz, 2H), 2.87 (s, 3H) |
| I-54 | 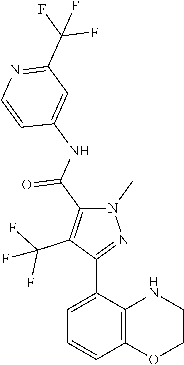 [SMILES: Cn1nc(-c2cccc3c2NCCO3)c(C(F)(F)F)c1C(=O)Nc1ccnc(C(F)(F)F)c1] | 5-(4,4,5,5- tetramethyl- 1,3,2- dioxaborolan-2- yl)-3,4-dihydro- 2H- benzo[b][1,4]oxa- zine | (ES, m/z): [M + H]⁺ 472.2 ¹H NMR (400 MHz, DMSO) δ 11.87 (s, 1H), 8.73 (s, 1H), 8.18 (s, 1H), 7.87 (s, 1H), 6.79-6.70 (m, 2H), 6.56 (t, J = 7.8 Hz, 1H), 5.70 (s, 1H), 4.12 (t, J = 4.3 Hz, 2H), 3.97 (s, 3H), 3.39-3.23 (m, 2H, peak partly covered by the water residual peak) |
| I-55 | 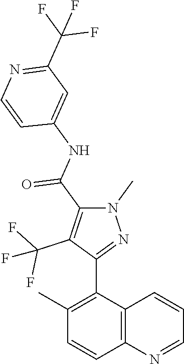 [SMILES: Cc1ccc2ncccc2c1-c1nn(C)c(C(=O)Nc2ccnc(C(F)(F)F)c2)c1C(F)(F)F] | (6- methylquinolin- 5-yl)boronic acid | (ES, m/z): [M + H]⁺ 480.2 ¹H NMR (400 MHz, DMSO) δ 11.84 (s, 1H), 8.90 (dd, J = 4.1, 1.6 Hz, 1H), 8.82-8.74 (m, 1H), 8.21 (s, 1H), 8.09 (d, J = 8.7 Hz, 1H), 7.98-7.91 (m, 1H), 7.78 (d, J = 8.7 Hz, 1H), 7.71 (d, J = 8.7 Hz, 1H), 7.52 (dd, J = 8.5, 5.0 Hz, 1H), 4.08 (s, 3H), 2.32 (s, 3H) |
| I-56 | 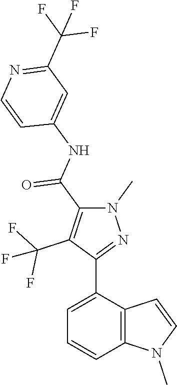 [SMILES: Cn1nc(-c2cccc3c2ccn3C)c(C(F)(F)F)c1C(=O)Nc1ccnc(C(F)(F)F)c1] | 1-methyl-4- (4,4,5,5- tetramethyl- 1,3,2- dioxaborolan-2- yl)-1H-indole | (ES, m/z): [M + H]⁺ 468.3 ¹H NMR (400 MHz, DMSO) δ 11.91 (s, 1H), 8.75 (s, 1H), 8.20 (s, 1H), 7.90 (s, 1H), 7.55 (d, J = 8.2 Hz, 1H), 7.40 (d, J = 3.1 Hz, 1H), 7.26 (t, J = 7.7 Hz, 1H), 7.15 (d, J = 7.3 Hz, 1H), 6.54 (d, J = 3.1 Hz, 1H), 4.02 (s, 3H), 3.84 (s, 3H) |
| I-57 | 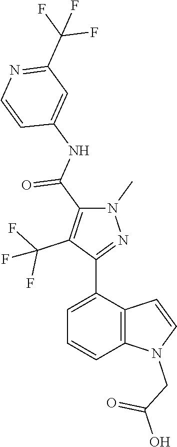 [SMILES: Cn1nc(-c2cccc3c2ccn3CC(=O)O)c(C(F)(F)F)c1C(=O)Nc1ccnc(C(F)(F)F)c1] | ethyl 2-(4- (4,4,5,5- tetramethyl- 1,3,2- dioxaborolan-2- yl)-1H-indol-1- yl)acetate | (ES, m/z): [M + H]⁺ 512.5 ¹H NMR (400 MHz, DMSO) δ 8.75 (d, J = 5.4 Hz, 1H), 8.21 (d, J = 1.7 Hz, 1H), 7.91 (dd, J = 5.4, 1.7 Hz, 1H), 7.45 (d, J = 8.1 Hz, 1H), 7.38 (d, J = 3.1 Hz, 1H), 7.20 (t, J = 7.7 Hz, 1H), 7.13 (d, J = 7.2 Hz, 1H), 6.55 (d, J = 3.1 Hz, 1H), 4.85 (s, 2H), 4.03 (s, 3H), NH and OH not present |
| I-58 | 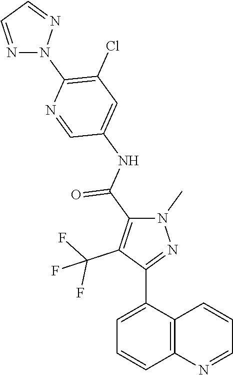 [SMILES: Cn1nc(-c2cccc3ncccc23)c(C(F)(F)F)c1C(=O)Nc1cnc(-n2nccn2)c(Cl)c1] | quinolin-5- ylboronic acid | (ES, m/z): [M + H]⁺ 498.9,, ¹H NMR (400 MHz, DMSO) d 11.96-11.90 (m, 1H), 8.99 (dd, J = 1.6, 4.2 Hz, 1H), 8.79 (s, 1H), 8.66 (d, J = 2.3 Hz, 1H), 8.19 (d, J = 9.1 Hz, 4H), 7.92-7.87 (m, 1H), 7.67 (d, J = 7.1 Hz, 1H), 7.61 (dd, J = 4.0, 8.6 Hz, 1H), 4.10 (s, 3H). |
| I-59 | 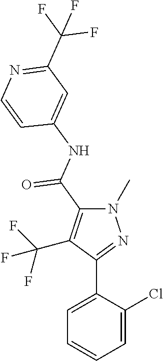 [SMILES: Cn1nc(-c2ccccc2Cl)c(C(F)(F)F)c1C(=O)Nc1ccnc(C(F)(F)F)c1] | (2- chlorophenyl)bo- ronic acid | (ES, m/z): [M + H]⁻ 448.8, ¹H NMR (400 MHz, DMSO) δ 11.90 (s, 1H), 8.75 (d, J = 5.4 Hz, 1H), 8.19 (s, 1H), 7.90 (d, J = 4.4 Hz, 1H), 7.62 (dd, J = 7.9, 1.0 Hz, 1H), 7.54 (dt, J = 7.3, 1.8 Hz, 1H), 7.47 (dt, J = 7.5, 1.2 Hz, 1H), 7.42 (dd, J = 7.6, 1.8 Hz, 1H), 4.02 (s, 3H) |
| I-60 | 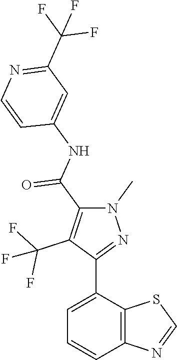 [SMILES: Cn1nc(-c2cccc3ncsc23)c(C(F)(F)F)c1C(=O)Nc1ccnc(C(F)(F)F)c1] | 7-(4,4,5,5- tetramethyl- 1,3,2- dioxaborolan-2- yl)benzo[d]thiazole | (ES, m/z): [M + H]⁻ 471.4, ¹H NMR (400 MHz, DMSO) δ 12.00 (s, 1H), 9.49 (s, 1H), 8.77 (d, J = 5.3 Hz, 1H), 8.29-8.14 (m, 2H), 7.90 (d, J = 4.4 Hz, 1H), 7.78-7.65 (m, 2H), 4.08 (s, 3H) |
| I-61 | 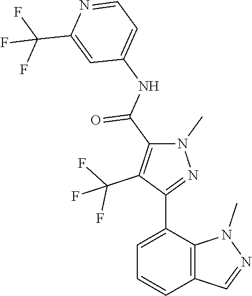 [SMILES: Cn1nc(-c2cccc3cnn(C)c23)c(C(F)(F)F)c1C(=O)Nc1ccnc(C(F)(F)F)c1] | 1-methyl-7- (4,4,5,5- tetramethyl- 1,3,2- dioxaborolan-2- yl)-1H-indazole | (ES, m/z): [M + H]⁺ 469.2, ¹H NMR (400 MHz, DMSO) 11.89 (s, 1H), 8.76 (s, 1H), 8.20 (s, 1H), 8.18 (s, 1H), 7.92 (dd, J = 1.0, 8.1 Hz, 2H), 7.33 (d, J = 6.8 Hz, 1H), 7.27-7.22 (m, 1H), 4.06 (s, 3H), 3.66 (s, 3H) |
| I-62 | 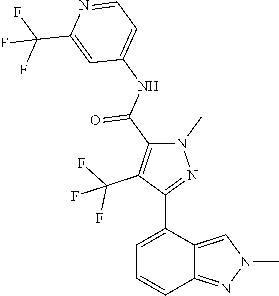 [SMILES: Cn1cc2c(-c3nn(C)c(C(=O)Nc4ccnc(C(F)(F)F)c4)c3C(F)(F)F)cccc2n1] | 2-methyl-4- (4,4,5,5- tetramethyl- 1,3,2- dioxaborolan-2- yl)-2H-indazole | (ES, m/z): [M + H]⁺ 469.2, ¹H NMR (400 MHz, DMSO) 11.98 (s, IH), 8.77 (d, J = 5.5 Hz, 1H), 8.48 (s, 1H), 8.21 (d, J = 1.6 Hz, 1H), 7.90 (dd, J = 1.6, 5.5 Hz, 1H), 7.72-7.69 (m, 1H), 7.37 (dd, J = 7.0, 8.7 Hz, 1H), 7.25 (d, J = 6.9 Hz, 1H), 4.22 (s, 3H), 4.06 (s, 3H) |
| I-63 | 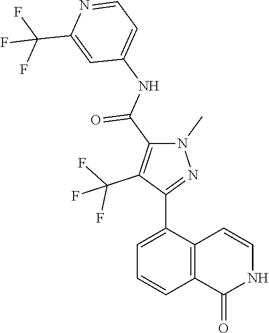 [SMILES: Cn1nc(-c2cccc3c(=O)[nH]ccc23)c(C(F)(F)F)c1C(=O)Nc1ccnc(C(F)(F)F)c1] | 5-(4,4,5,5- tetramethyl- 1,3,2- dioxaborolan-2- yl)isoquinolin- 1(2H)-one | (ES, m/z): [M + H]⁺ 482.2, ¹H NMR (400 MHz, DMSO) δ 11.94-11.93 (m, 1H), 11.45 (d, J = 5.4 Hz, 1H), 8.76 (d, J = 5.4 Hz, 1H), 8.36-8.33 (m, 1H), 8.20 (d, J = 1.3 Hz, 1H), 7.89 (d, J = 4.6 Hz, 1H), 7.69 (d, J = 6.4 Hz, 1H), 7.60 (t, J = 7.7 Hz, 1H), 7.22 (dd, J = 6.0, 7.3 Hz, 1H), 6.29 (d, J = 7.3 Hz, 1H), 4.05 (s, 3H) |
| I-64 | 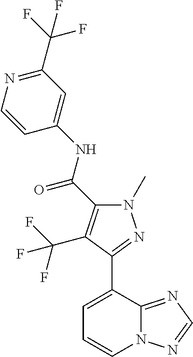 [SMILES: Cn1nc(-c2cccn3ncnc23)c(C(F)(F)F)c1C(=O)Nc1ccnc(C(F)(F)F)c1] | [1,2,4]triazolo[1,5- a]pyridin-8- ylboronic acid | (ES, m/z): [M + H]⁺ 456.5, ¹H NMR (400 MHz, DMSO) δ d 11.99 (s, 1H), 9.11 (d, J = 6.8 Hz, 1H), 8.75 (d, J = 5.1 Hz, 1H), 8.56-8.55 (m, 1H), 8.20 (d, J = 1.5 Hz, 1H), 7.89 (d, J = 4.0 Hz, 1H), 7.75-7.72 (m, 1H), 7.34 (t, J = 7.1 Hz, 1H), 4.05 (s, 3H) |
| I-65 | 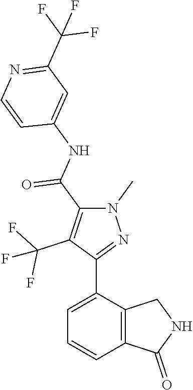 [SMILES: Cn1nc(-c2cccc3c2CNC3=O)c(C(F)(F)F)c1C(=O)Nc1ccnc(C(F)(F)F)c1] | 4-(4,4,5,5- tetramethyl- 1,3,2- dioxaborolan-2- yl)isoindolin-1- one | (ES, m/z): [M + H]⁺ 470.4 ¹H NMR (400 MHz, DMSO) δ d 11.94 (s, 1H), 8.77-8.70 (m, 2H), 8.18 (s, IH), 7.88 (d, J = 3.0 Hz, 1H), 7.80 (dd, J = 1.3, 7.1 Hz, 1H), 7.72-7.64 (m, 2H), 4.49 (s, 2H), 4.03-4.02 (m, 3H) |
| I-66 | 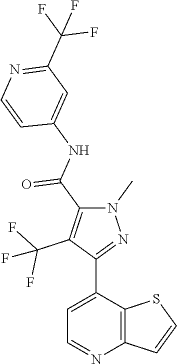 [SMILES: Cn1nc(-c2ccnc3ccsc23)c(C(F)(F)F)c1C(=O)Nc1ccnc(C(F)(F)F)c1] | thieno[3,2- b]pyridin-7- ylboronic acid | (ES, m/z): [M + H]⁺ 472.2, ¹H NMR (400 MHz, DMSO) δ 12.03 (s, 1H), 8.84 (d, J = 4.8 Hz, 1H), 8.76 (d, J = 4.4 Hz, 1H), 8.26 (d, J = 5.6 Hz, 1H), 8.19 (s, 1H), 7.89 (s, 1H), 7.68 (d, J = 5.6 Hz, 1H), 7.52 (d, J = 4.8 Hz, 1H), 4.10 (s, 3H) |
| I-67 | 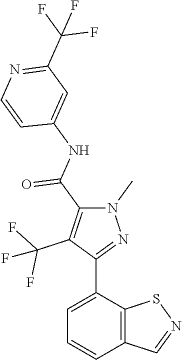 [SMILES: Cn1nc(-c2cccc3cnsc23)c(C(F)(F)F)c1C(=O)Nc1ccnc(C(F)(F)F)c1] | benzo[d]isothiazol- 7-ylboronic acid | (ES, m/z): [M + H]⁺ 472.2, ¹H NMR (400 MHz, DMSO) δ 12.06 (s, 1H), 9.28 (s, 1H), 8.82 (d, J = 5.6 Hz, 1H), 8.41 (d, J = 7.8 Hz, 1H), 8.25 (d, J = 1.8 Hz, 1H), 7.98 (d, J = 7.7 Hz, 1H), 7.94 (dd, J = 1.5, 5.4 Hz, 1H), 7.79 (dd, J = 7.7, 7.7 Hz, 1H), 4.16 (s, 3H) |
| I-68 | 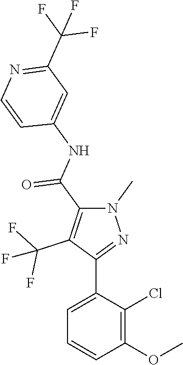 [SMILES: COc1cccc(-c2nn(C)c(C(=O)Nc3ccnc(C(F)(F)F)c3)c2C(F)(F)F)c1Cl] | (2-chloro-3- methoxyphenyl) boronic acid | (ES, m/z): [M + H]⁺ 479.2, ¹H NMR (400 MHz, DMSO) δ 11.88 (s, 1H), 8.74 (d, J = 5.1 Hz, IH), 8.19 (s, 1H), 7.92- 7.87 (m, 1H), 7.42 (dd, J = 8.0, 8.0 Hz, 1H), 7.31-7.27 (m, 1H), 6.98 (dd, J = 1.3, 7.6 Hz, 1H), 4.01 (s, 3H), 3.91 (s, 3H) |
| I-69 | 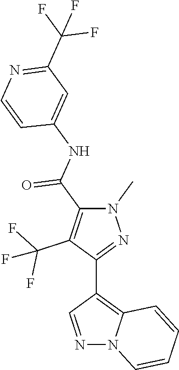 [SMILES: Cn1nc(-c2cnn3ccccc23)c(C(F)(F)F)c1C(=O)Nc1ccnc(C(F)(F)F)c1] | 3-(4,4,5,5- tetramethyl- 1,3,2- dioxaborolan-2- yl)pyrazolo[1,5- a]pyridine | (ES, m/z): [M + H]⁺ 455.2, ¹H NMR (400 MHz, DMSO) δ 11.92 (s, 1H), 8.81 (d, J = 6.8 Hz, 1H), 8.74 (d, J = 4.8 Hz, 1H), 8.19 (s, 2H), 8.13 (d, J = 9.1 Hz, 1H), 7.89-7.85 (m, 1H), 7.47-7.43 (m, 1H), 7.09-7.04 (m, 1H), 4.00 (s, 3H) |
| I-70 | 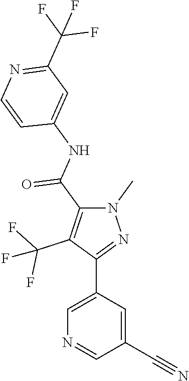 [SMILES: Cn1nc(-c2cncc(C#N)c2)c(C(F)(F)F)c1C(=O)Nc1ccnc(C(F)(F)F)c1] | 5-(4,4,5,5- tetramethyl- 1,3,2- dioxaborolan-2- yl)nicotinonitrile | (ES, m/z): [M + H]⁺ 441.2, ¹H NMR (400 MHz, DMSO) δ 11.99 (s, 1H), 9.18 (d, J = 1.8 Hz, 1H), 9.02 (d, J = 2.0 Hz, 1H), 8.75 (d, J = 5.3 Hz, 1H), 8.48 (t, J = 1.9 Hz, 1H), 8.18 (d, J = 1.5 Hz, 1H), 7.87 (d, J = 4.0 Hz, 1H), 4.05 (s, 3H) |
| I-71 | 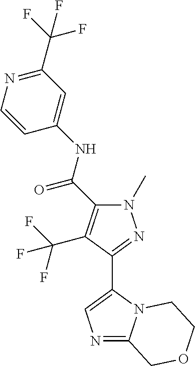 [SMILES: Cn1nc(-c2cnc3n2CCOC3)c(C(F)(F)F)c1C(=O)Nc1ccnc(C(F)(F)F)c1] | 3-(4,4,5,5- tetramethyl- 1,3,2- dioxaborolan-2- yl)-5,6-dihydro- 8H-imidazo[2,1- c][1,4]oxazine | (ES, m/z): [M + H]⁺ 461.2, ¹H NMR (400 MHz, DMSO) δ 11.94 (s, 1H), 8.74 (d, J = 4.3 Hz, 1H), 8.16 (s, 1H), 7.85 (s, 1H), 7.18 (s, 1H), 4.82 (s, 2H), 4.18-4.13 (m, 2H), 4.08-4.04 (m, 2H), 3.97 (s, 3H) |
| I-72 | 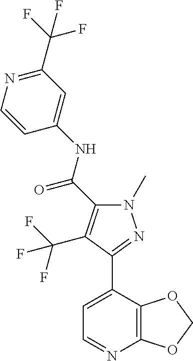 [SMILES: Cn1nc(-c2ccnc3c2OCO3)c(C(F)(F)F)c1C(=O)Nc1ccnc(C(F)(F)F)c1] | 7-(4,4,5,5- tetramethyl- 1,3,2- dioxaborolan-2- yl)- [1,3]dioxolo[4,5- b]pyridine | (ES, m/z): [M + H]⁺ 460.2, ¹H NMR (400 MHz, DMSO) δ 11.93 (s, 1H), 8.74 (s, 1H), 8.17 (s, 1H), 7.86-7.85 (m, 1H), 7.71 (d, J = 5.6 Hz, 1H), 6.93 (d, J = 5.6 Hz, IH), 6.21 (s, 2H), 4.02 (s, 3H) |
| I-73 | 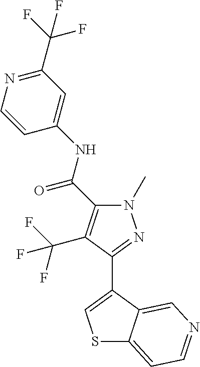 [SMILES: Cn1nc(-c2csc3ccncc23)c(C(F)(F)F)c1C(=O)Nc1ccnc(C(F)(F)F)c1] | thieno[3,2- c]pyridin-3- ylboronic acid | (ES, m/z): [M + H]⁺ 472.2, ¹H NMR (400 MHz, DMSO) δ 11.98 (s, 1H), 9.37 (s, 1H), 8.74 (d, J = 4.3 Hz, 1H), 8.52 (d, J = 5.6 Hz, 1H), 8.20-8.17 (m, 2H), 8.10 (s, 1H), 7.88 (d, J = 4.5 Hz, 1H), 4.07 (s, 3H) |
| I-74 | 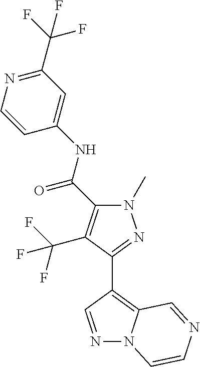 [SMILES: Cn1nc(-c2cnn3ccncc23)c(C(F)(F)F)c1C(=O)Nc1ccnc(C(F)(F)F)c1] | 3-(4,4,5,5- tetramethyl- 1,3,2- dioxaborolan-2- yl)pyrazolo[1,5- a]pyrazine | (ES, m/z): [M + H]⁺ 456.2, ¹H NMR (400 MHz, DMSO) δ 11.95 (s, 1H), 9.56 (d, J = 1.5 Hz, 1H), 8.92 (dd, J = 1.4, 4.7 Hz, 1H), 8.75 (d, J = 5.3 Hz, 1H), 8.35 (s, 1H), 8.19 (d, J = 1.5 Hz, 1H), 8.09 (d, J = 4.8 Hz, 1H), 7.88 (d, J = 5.3 Hz, 1H), 4.05 (s, 3H) |
| I-75 | 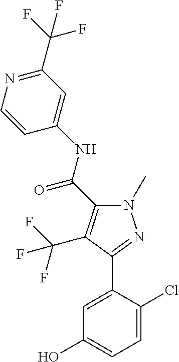 [SMILES: Cn1nc(-c2cc(O)ccc2Cl)c(C(F)(F)F)c1C(=O)Nc1ccnc(C(F)(F)F)c1] | (2-chloro-5- hydroxyphenyl) boronic acid | (ES, m/z): [M + H]⁺ 465.2, ¹H NMR (400 MHz, DMSO) δ 11.92 (s, 1H), 10.01 (s, 1H), 8.80 (d, J = 5.3 Hz, 1H), 8.24 (d, J = 1.3 Hz, 1H), 7.95 (d, J = 4.0 Hz, 1H), 7.43 (d, J = 8.8 Hz, 1H), 6.96 (dd, J = 2.9, 8.7 Hz, 1H), 6.83 (d, J = 2.8 Hz, 1H), 4.06 (s, 3H) |
| I-76 | 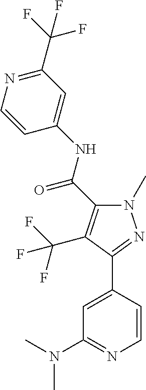 [SMILES: CN(C)c1cc(-c2nn(C)c(C(=O)Nc3ccnc(C(F)(F)F)c3)c2C(F)(F)F)ccn1] | N,N-dimethyl-4- (4,4,5,5- tetramethyl- 1,3,2- dioxaborolan-2- yl)pyridin-2- amine | (ES, m/z): [M + H]⁺ 459.2, ¹H NMR (400 MHz, DMSO) δ 11.93 (s, 1H), 8.73 (d, J = 5.6 Hz, 1H), 8.20 (d, J = 5.1 Hz, 1H), 8.16 (s, 1H), 7.84 (d, J = 3.3 Hz, 1H), 6.77-6.72 (m, 2H), 3.99 (s, 3H), 3.06 (s, 6H) |
| I-77 | 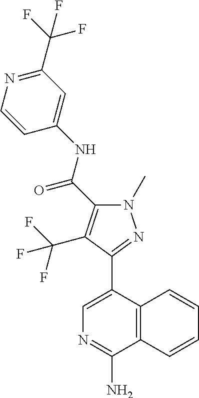 [SMILES: Cn1nc(-c2cnc(N)c3ccccc23)c(C(F)(F)F)c1C(=O)Nc1ccnc(C(F)(F)F)c1] | tert-butyl (tert- butoxycarbonyl) (4-(4,4,5,5- tetramethyl- 1,3,2- dioxaborolan-2- yl)isoquinolin- 1-yl)carbamate | (ES, m/z): [M − H]− 479.2, ¹H NMR (400 MHz, DMSO) δ 11.88 (s, 1H), 8.75 (d, J = 4.4 Hz, 1H), 8.28 (d, J = 8.3 Hz, 1H), 8.20 (s, 1H), 7.90 (br s, 1H), 7.79 (s, 1H), 7.66 (ddd, J = 8.3, 6.8, 1.0 Hz, 1H), 7.59 (d, J = 7.7 Hz, 1H), 7.53 (ddd, J = 8.2, 6.8, 1.2 Hz, 1H), 7.11 (s, 2H), 4.03 (s, 3H) |
| I-78 | 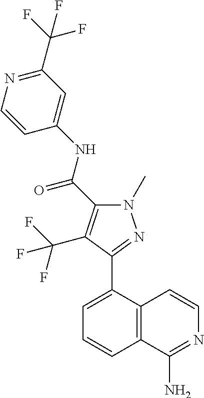 [SMILES: Cn1nc(-c2cccc3c(N)nccc23)c(C(F)(F)F)c1C(=O)Nc1ccnc(C(F)(F)F)c1] | tert-butyl (tert- butoxycarbonyl) (5-(4,4,5,5- tetramethyl- 1,3,2- dioxaborolan-2- yl)isoquinolin- 1-yl)carbamate | (ES, m/z): [M + H]⁻ 481.2, ¹H NMR (400 MHz, DMSO) δ 11.92 (br s, 1H), 8.76 (d, J = 5.5 Hz, 1H), 8.33 (d, J = 8.2 Hz, 1H), 8.20 (d, J = 1.6 Hz, 1H), 8.16 (formate peak, 0.5H), 7.92 (dd, J = 5.5, 1.6 Hz, 1H), 7.81 (d, J = 6.0 Hz, 1H), 7.63-7.52 (m, 2H), 6.93 (s, 2H), 6.66 (d, J = 6.0 Hz, 1H), 4.04 (s, 3H) |
| I-79 | 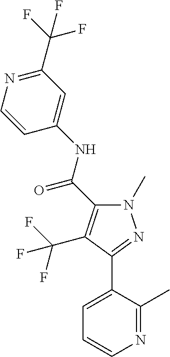 [SMILES: Cc1ncccc1-c1nn(C)c(C(=O)Nc2ccnc(C(F)(F)F)c2)c1C(F)(F)F] | (2- methylpyridin- 3-yl)boronic acid | (ES, m/z): [M + H]⁺ 430.2, ¹H NMR (400 MHz, DMSO) δ 11.92-11.88 (m, 1H), 8.75 (d, J = 5.3 Hz, 1H), 8.57 (dd, J = 1.8, 4.8 Hz, 1H), 8.18 (s, 1H), 7.91- 7.87 (m, 1H), 7.65 (dd, J = 1.3, 7.8 Hz, 1H), 7.35 (dd, J = 4.9, 7.5 Hz, 1H), 4.03-4.01 (m, 3H), 2.40-2.39 (m, 3H) |
| I-80 | 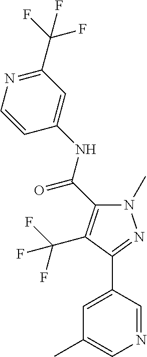 [SMILES: Cc1cncc(-c2nn(C)c(C(=O)Nc3ccnc(C(F)(F)F)c3)c2C(F)(F)F)c1] | (5- methylpyridin- 3-yl)boronic acid | (ES, m/z): [M + H]⁺ 430.2, ¹H NMR (400 MHz, DMSO) δ 11.94 (s, 1H), 8.74 (d, J = 5.3 Hz, 1H), 8.55 (dd, J = 1.6, 9.5 Hz, 2H), 8.18 (s, 1H), 7.87- 7.83 (m, 1H), 7.79 (s, 1H), 4.01 (s, 3H), 2.39 (s, 3H) |
| I-81 | 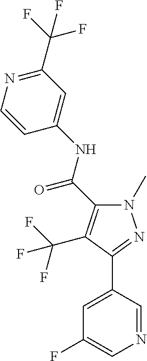 [SMILES: Cn1nc(-c2cncc(F)c2)c(C(F)(F)F)c1C(=O)Nc1ccnc(C(F)(F)F)c1] | (5- fluoropyridin-3- yl)boronic acid | (ES, m/z): [M + H]⁻ 434.5, ¹H NMR (400 MHz, DMSO), ¹H NMR (400 MHz, DMSO) δ 11.97 (s, 1H), 8.77-8.74 (m, 2H), 8.65 (s, 1H), 8.18 (d, J = 1.5 Hz, 1H), 7.92-7.85 (m, 2H), 4.04 (s, 3H) |
| I-82 | 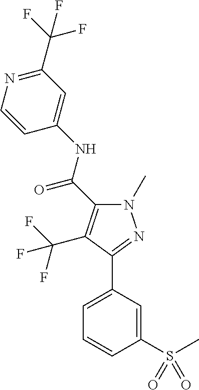 [SMILES: Cn1nc(-c2cccc(S(C)(=O)=O)c2)c(C(F)(F)F)c1C(=O)Nc1ccnc(C(F)(F)F)c1] | (3- (methylsulfonyl) phenyl)boronic acid | (ES, m/z): [M + H]⁺ 493.6,, ¹H NMR (400 MHz, DMSO) δ 11.93 (s, 1H), 8.76 (d, J = 5.6 Hz, 1H), 8.20-8.06 (m, 3H), 7.96-7.81 (m, 3H), 4.03 (s, 3H), 3.29 (s, 3H) |
| I-83 | 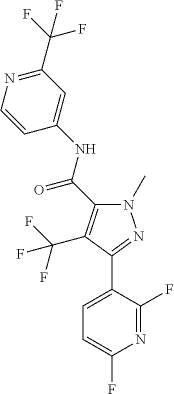 [SMILES: Cn1nc(-c2ccc(F)nc2F)c(C(F)(F)F)c1C(=O)Nc1ccnc(C(F)(F)F)c1] | (2,6- difluoropyridin- 3-yl)boronic acid | (ES, m/z): [M + H]⁺ 452.5, ¹H NMR (400 MHz, DMSO) δ 11.94-11.90 (m, 1H), 8.77- 8.72 (m, 1H), 8.35-8.27 (m, 1H), 8.17 (s, 1H), 7.87 (dd, J = 13.3, 13.3 Hz, 1H), 7.38 (dd, J = 2.3, 8.1 Hz, 1H), 4.04 (s, 3H) |
| I-84 | 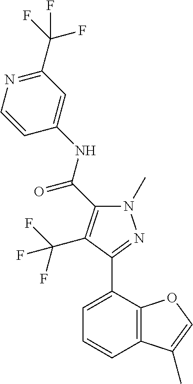 [SMILES: Cc1coc2c(-c3nn(C)c(C(=O)Nc4ccnc(C(F)(F)F)c4)c3C(F)(F)F)cccc12] | 4,4,5,5- tetramethyl-2- (3- methylbenzofuran- 7-yl)-1,3,2- dioxaborolane | (ES, m/z): [M + H]⁺ 469.2, ¹H NMR (400 MHz, DMSO) δ 11.96 (s, 1H), 8.76 (d, J = 5.5 Hz, 1H), 8.21 (d, J = 1.8 Hz, 1H), 7.90 (dd, J = 1.7, 5.5 Hz, 1H), 7.83 (d, J = 1.4 Hz, 1H), 7.76-7.72 (m, 1H), 7.39-7.37 (m, 2H), 4.04 (s, 3H), 2.26 (d, J = 1.3 Hz, 3H) |
| I-87 | 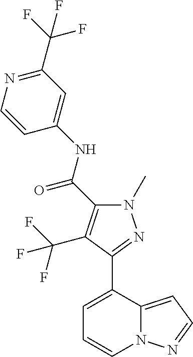 [SMILES: Cn1nc(-c2cccn3nccc23)c(C(F)(F)F)c1C(=O)Nc1ccnc(C(F)(F)F)c1] | 4-(4,4,5,5- tetramethyl- 1,3,2- dioxaborolan-2- yl)pyrazolo[1,5- a]pyridine | (ES, m/z): [M + H]⁺ 455.5, ¹H NMR (400 MHz, DMSO) δ 11.95 (s, 1H), 8.83-8.73 (m, 2H), 8.18 (s, 1H), 8.09 (d, J = 2.3 Hz, 1H), 7.89-7.86 (m, 1H), 7.34 (d, J = 6.8 Hz, 1H), 7.04 (t, J = 6.9 Hz, 1H), 6.73 (d, J = 1.5 Hz, 1H), 4.06 (s, 3H) |
| I-88 | 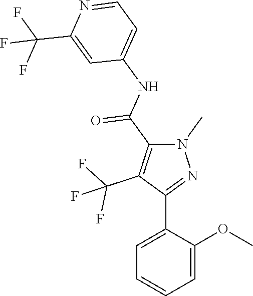 [SMILES: COc1ccccc1-c1nn(C)c(C(=O)Nc2ccnc(C(F)(F)F)c2)c1C(F)(F)F] | (2- methoxyphenyl) boronic acid | (ES, m/z): [M + H]⁺ 445.2, ¹H NMR (400 MHz, DMSO) δ d 11.87 (s, 1H), 8.74 (d, J = 5.5 Hz, 1H), 8.19 (d, J = 1.5 Hz, 1H), 7.90 (d, J = 4.0 Hz, 1H), 7.49-7.44 (m, 1H), 7.28 (dd, J = 1.7, 7.5 Hz, 1H), 7.15-7.12 (m, 1H), 7.03 (dd, J = 6.6, 7.5 Hz, 1H), 3.98 (s, 3H), 3.76 (s, 3H) |
| I-89 | 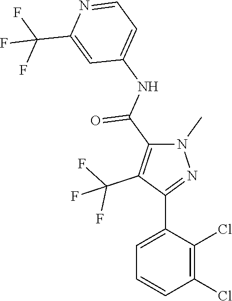 [SMILES: Cn1nc(-c2cccc(Cl)c2Cl)c(C(F)(F)F)c1C(=O)Nc1ccnc(C(F)(F)F)c1] | (2,3- dichlorophenyl) boronic acid | (ES, m/z): [M + H]⁺ 483.2, ¹H NMR (400 MHz, DMSO) δ 12.02-11.90 (m, 1H), 8.75- 8.70 (m, IH), 8.19-8.16 (m, 1H), 7.90-7.85 (m, 1H), 7.83- 7.80 (m, 1H), 7.53-7.48 (m, 1H), 7.44-7.40 (m, 1H), 4.02 (s, 3H) |
| I-90 | 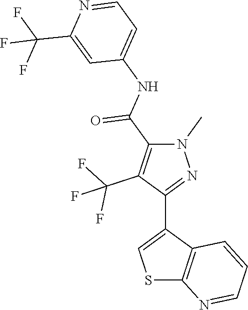 [SMILES: Cn1nc(-c2csc3ncccc23)c(C(F)(F)F)c1C(=O)Nc1ccnc(C(F)(F)F)c1] | 3-(4,4,5,5- tetramethyl- 1,3,2- dioxaborolan-2- yl)thieno[2,3- b]pyridine | (ES, m/z): [M + H]⁺ 472.4, ¹H NMR (400 MHz, DMSO) 11.97 (s, 1H), 8.77 (d, J = 5.4 Hz, 1H), 8.69 (dd, J = 1.6, 4.5 Hz, 1H), 8.50 (dd, J = 1.6, 8.2 Hz, 1H), 8.21 (d, J = 1.6 Hz, 1H), 8.14 (s, 1H), 7.90 (dd, J = 1.7, 5.5 Hz, 1H), 7.57 (dd, J = 4.5, 8.2 Hz, 1H), 4.07 (s, 3H) |
| I-91 | 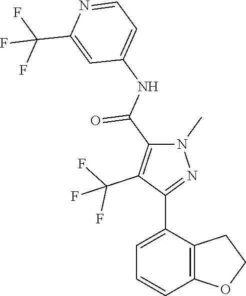 [SMILES: Cn1nc(-c2cccc3c2CCO3)c(C(F)(F)F)c1C(=O)Nc1ccnc(C(F)(F)F)c1] | 2-(2,3- dihydrobenzofuran- 4-yl)-4,4,5,5- tetramcthyl- 1,3,2- dioxaborolane | (ES, m/z): [M + H]⁺ 457.2, ¹H NMR (400 MHz, DMSO) 11.92 (s, 1H), 8.76 (d, J = 5.4 Hz, 1H), 8.19 (d, J = 1.4 Hz, 1H), 7.89 (dd, J = 1.6, 5.5 Hz, 1H), 7.23 (t, J = 7.8 Hz, 1H), 6.89 (dd, J = 8.0, 9.9 Hz, 2H), 4.57 (t, J = 8.7 Hz, 2H), 4.00- 3.99 (m, 3H), 3.25 (t, J = 8.7 Hz, 2H) |
| I-92 | 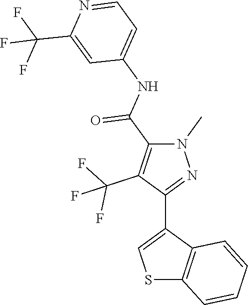 [SMILES: Cn1nc(-c2csc3ccccc23)c(C(F)(F)F)c1C(=O)Nc1ccnc(C(F)(F)F)c1] | benzo[b]thiophen- 3-ylboronic acid | (ES, m/z): [M + H]⁺ 471.4, ¹H NMR (400 MHz, DMSO) 11.95 (s, 1H), 8.76 (d, J = 5.6 Hz, 1H), 8.20 (d, J = 1.8 Hz, 1H), 8.13-8.07 (m, 1H), 8.08- 8.02 (m, 1H), 7.97 (s, 1H), 7.90 (dd, J = 1.8, 5.6 Hz, 1H), 7.51- 7.44 (m, 2H), 4.06 (s, 3H) |
| I-93 | 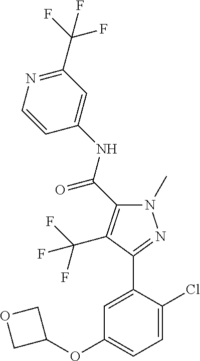 [SMILES: Cn1nc(-c2cc(OC3COC3)ccc2Cl)c(C(F)(F)F)c1C(=O)Nc1ccnc(C(F)(F)F)c1] | 2-(2-chloro-5- (oxetan-3- yloxy)phenyl)- 4,4,5,5- tetramethyl- 1,3,2- dioxaborolane | (ES, m/z): [M + H]⁺ 521.2, ¹H NMR (400 MHz, DMSO) δ 11.89 (s, 1H), 8.75 (d, J = 5.3 Hz, 1H), 8.20-8.18 (m, 1H), 7.93-7.89 (m, 1H), 7.52 (d, J = 8.8 Hz, 1H), 6.98 (dd, J = 3.0, 8.8 Hz, 1H), 6.80 (d, J = 3.0 Hz, 1H), 5.36-5.29 (m, 1H), 4.93- 4.89 (m, 2H), 4.55 (dd, J = 4.9, 7.7 Hz, 2H), 4.02 (s, 3H). |
| I-94 | 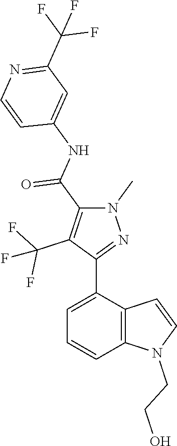 [SMILES: Cn1nc(-c2cccc3c2ccn3CCO)c(C(F)(F)F)c1C(=O)Nc1ccnc(C(F)(F)F)c1] | 2-(4-(4,4,5,5- tetramethyl- 1,3,2- dioxaborolan-2- yl)-1H-indol-1- yl)ethan-1-ol | (ES, m/z): [M + H]⁻ 498.2, ¹H NMR (400 MHz, DMSO) δ 11.92 (s, 1H), 8.75 (d, J = 5.3 Hz, 1H), 8.20 (s, 1H), 7.90 (d, J = 5.3 Hz, 1H), 7.59 (d, J = 8.2 Hz, 1H), 7.44 (d, J = 3.1 Hz, 1H), 7.23 (t, J = 7.7 Hz, 1H), 7.13 (d, J = 7.2 Hz, 1H), 6.55 (d, J = 3.1 Hz, 1H), 4.91 (t, J = 5.3 Hz, 1H), 4.27 (t, J = 5.5 Hz, 2H), 4.02 (s, 3H), 3.75 (q, J = 5.4 Hz, 2H) |
| I-95 | 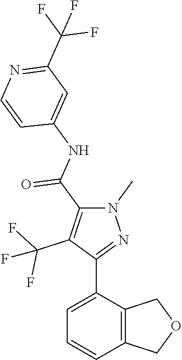 [SMILES: Cn1nc(-c2cccc3c2COC3)c(C(F)(F)F)c1C(=O)Nc1ccnc(C(F)(F)F)c1] | 2-(1,3- dihydroisobenzo- furan-4-yl)- 4,4,5,5- tetramethyl- 1,3,2- dioxaborolane | (ES, m/z): [M + H] 457.6, ¹H NMR (400 MHz, DMSO) δ 11.91 (s, 1H), 8.75 (d, J = 5.4 Hz, 1H), 8.18 (s, 1H), 7.88 (d, J = 4.5 Hz, 1H), 7.50-7.33 (m, 3H), 5.11 (s, 2H), 5.09 (s, 2H), 3.99 (s, 3H) |
| I-96 | 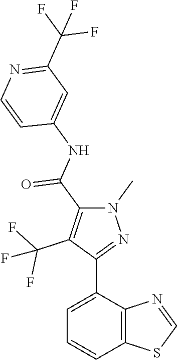 [SMILES: Cn1nc(-c2cccc3scnc23)c(C(F)(F)F)c1C(=O)Nc1ccnc(C(F)(F)F)c1] | 4-(4,4,5,5- tetramethyl- 1,3,2- dioxaborolan-2- yl)benzo[d]thiazole | (ES, m/z): [M + H]⁻ 472.2, ¹H NMR (400 MHz, DMSO) δ 11.97 (br s, 1H), 9.40 (s, 1H), 8.72 (br s, 1H), 8.31 (dd, J = 1.6, 7.8 Hz, 1H), 8.20 (s, 1H), 7.89 (br s, 1H), 7.60 (t, J = 7.6 Hz, 1H), 7.55 (dd, J = 1.6, 7.3 Hz, 1H), 4.03 (s, 3H) |
| I-97 | 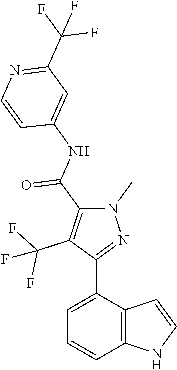 [SMILES: Cn1nc(-c2cccc3[nH]ccc23)c(C(F)(F)F)c1C(=O)Nc1ccnc(C(F)(F)F)c1] | (1H-indol-4- yl)boronic acid | (ES, m/z): [M + H]⁻ 454.2, ¹H NMR (400 MHz, DMSO) δ 11.84 (br s, 1H), 11.22 (s, 1H), 8.68 (d, J = 4.2 Hz, 1H), 8.13 (s, 1H), 7.83 (d, J = 4.2 Hz, 1H), 7.43 (d, J = 8.0 Hz, 1H), 7.34 (t, J = 2.7 Hz, 1H), 7.12 (t, J = 7.7 Hz, 1H), 7.04 (d, J = 7.2 Hz, 1H), 6.49 (s, 1H), 3.95 (s, 3H) |
| I-98 | 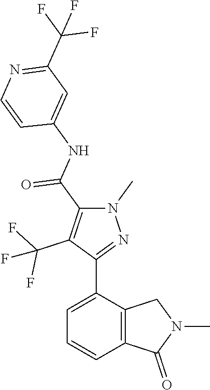 [SMILES: CN1Cc2c(cccc2-c2nn(C)c(C(=O)Nc3ccnc(C(F)(F)F)c3)c2C(F)(F)F)C1=O] | 2-methyl-4- (4,4,5,5- tetramethyl- 1,3,2- dioxaborolan-2- yl)isoindolin-1- one | (ES, m/z): [M + H]⁺ 484.2, ¹H NMR (400 MHz, DMSO) δ d 12.02 (s, 1H), 8.80 (s, 1H), 8.24- 8.22 (m, 1H), 7.92 (s, 1H), 7.86-7.82 (m, 1H), 7.74-7.70 (m, 2H), 4.63 (s, 2H), 4.08 (s, 3H), 3.16 (s, 3H) |
| I-99 | 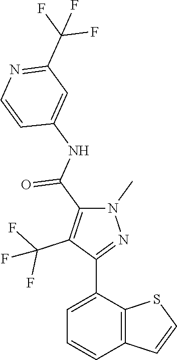 [SMILES: Cn1nc(-c2cccc3ccsc23)c(C(F)(F)F)c1C(=O)Nc1ccnc(C(F)(F)F)c1] | benzo[b]thiophen- 7-ylboronic acid | (ES, m/z): [M + H]⁺ 471.2, ¹H NMR (400 MHz, DMSO) δ 12.02-11.97 (m, 1H), 8.78- 8.74 (m, 1H), 8.19 (s, 1H), 8.01 (dd, J = 1.3, 7.6 Hz, 1H), 7.91- 7.83 (m, 2H), 7.57-7.50 (m, 3H), 4.06 (s, 3H) |
| I-100 | 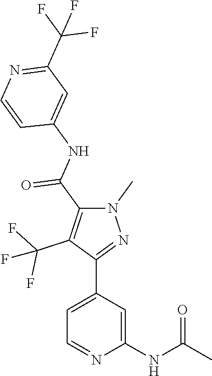 [SMILES: CC(=O)Nc1cc(-c2nn(C)c(C(=O)Nc3ccnc(C(F)(F)F)c3)c2C(F)(F)F)ccn1] | N-(4-(4,4,5,5- tetramethyl- 1,3,2- dioxaborolan-2- yl)pyridin-2- yl)acetamide | (ES, m/z): [M + H]⁺ 473.2, ¹H NMR (400 MHz, DMSO) δ 11.91 (s, 1H), 10.67-10.65 (m, 1H), 8.77-8.73 (m, IH), 8.46- 8.42 (m, 2H), 8.17 (s, 1H), 7.88- 7.85 (m, 1H), 7.28 (dd, J = 1.3, 5.1 Hz, 1H), 4.02 (s, 3H), 2.12- 2.11 (m, 3H) |
| I-101 | 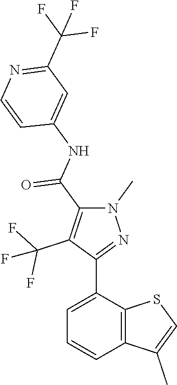 [SMILES: Cc1csc2c(-c3nn(C)c(C(=O)Nc4ccnc(C(F)(F)F)c4)c3C(F)(F)F)cccc12] | 4,4,5,5- tetramethyl-2- (3- methylbenzo[b]thi- ophen-7-yl)- 1,3,2- dioxaborolane | (ES, m/z): [M + H]⁺ 485.2, ¹H NMR (400 MHz, DMSO) δ 12.00-11.97 (m, 1H), 8.76 (d, J = 5.1 Hz, 1H), 8.19 (s, 1H), 7.92-7.88 (m, 2H), 7.60-7.55 (m, 1H), 7.52-7.47 (m, 2H), 4.05 (s, 3H), 2.46 (s, 3H) |
| I-102 | 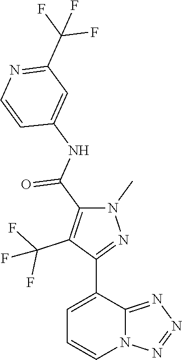 [SMILES: Cn1nc(-c2cccn3nnnc23)c(C(F)(F)F)c1C(=O)Nc1ccnc(C(F)(F)F)c1] | 8-(4,4,5,5- tetramethyl- 1,3,2- dioxaborolan-2- yl)tetrazolo[1,5- a]pyridine | (ES, m/z): [M + H]⁺ 457.2, ¹H NMR (400 MHz, DMSO) δ 12.03 (s, 1H), 9.47 (d, J = 6.3 Hz, 1H), 8.75 (s, 1H), 8.20 (s, 1H), 7.96 (d, J = 6.8 Hz, 1H), 7.89 (s, 1H), 7.60 (dd, J = 6.9, 6.9 Hz, 1H), 4.09 (s, 3H) |
| I-103 | 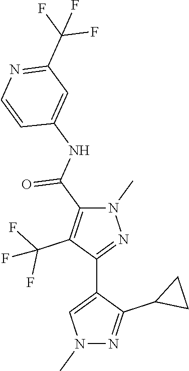 [SMILES: Cn1cc(-c2nn(C)c(C(=O)Nc3ccnc(C(F)(F)F)c3)c2C(F)(F)F)c(C2CC2)n1] | 3-cyclopropyl- 1-methyl-4- (4,4,5,5- tetramethyl- 1,3,2- dioxaborolan-2- yl)-1H-pyrazole | (ES, m/z): [M + H]⁺ 459.2, ¹H NMR (400 MHz, DMSO) δ 11.86 (s, 1H), 8.74 (d, J = 5.3 Hz, 1H), 8.17 (s, 1H), 7.87 (d, J = 3.5 Hz, 1H), 7.74 (s, 1H), 3.94 (s, 3H), 3.78 (s, 3H), 2.24- 2.16 (m, 1H), 0.86-0.74 (m, 4H) |
| I-104 | 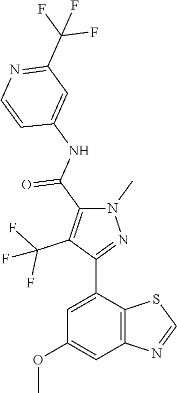 [SMILES: COc1cc(-c2nn(C)c(C(=O)Nc3ccnc(C(F)(F)F)c3)c2C(F)(F)F)c2scnc2c1] | 5-methoxy-7- (4,4,5,5- tetramethyl- 1,3,2- dioxaborolan-2- yl)benzo[d]thiazole | (ES, m/z): [M + H]⁻ 502.2, ¹H NMR (400 MHz, DMSO) δ 11.98 (br s, 1H), 9.45 (s, 1H), 8.73 (br s, 1H), 8.17 (s, 1H), 7.85 (br s, 1H), 7.77 (d, J = 2.0 Hz, 1H), 7.28 (s, 1H), 4.05 (s, 3H), 3.92 (s, 3H) |
| I-117 | 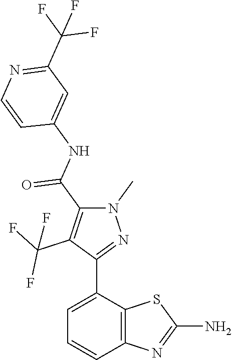 [SMILES: Cn1nc(-c2cccc3nc(N)sc23)c(C(F)(F)F)c1C(=O)Nc1ccnc(C(F)(F)F)c1] | 7-(4,4,5,5- tetramethyl- 1,3,2- dioxaborolan-2- yl)benzo[d]thiazol- 2-amine | (ES, m/z): [M + H]⁺ 487.2, ¹H NMR (400 MHz, DMSO) δ 11.95 (br s, 1H), 8.77-8.71 (m, 1H), 8.17 (s, 1H), 7.86 (s, 1H), 7.52-7.48 (m, 2H), 7.42 (dd, J = 1.1, 8.0 Hz, 1H), 7.35 (t, J = 7.7 Hz, 1H), 7.20 (d, J = 7.6 Hz, 1H), 4.01 (s, 3H) |
| I-223 | 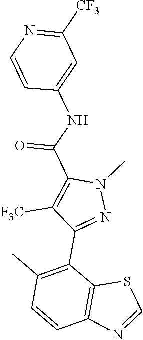 [SMILES: Cc1ccc2ncsc2c1-c1nn(C)c(C(=O)Nc2ccnc(C(F)(F)F)c2)c1C(F)(F)F] | 6-methyl-7- (4,4,5,5- tetramethyl- 1,3,2- dioxaborolan- 2-yl)-1,3- benzothiazole | (ES, m/z): [M + H]+ 486.2, ¹H NMR (400 MHz, DMSO) 11.92 (s, 1H), 9.32 (s, 1H), 8.75 (br s, 1H), 8.19 (s, 1H), 8.10 (d, J = 8.4 Hz, 1H), 7.91 (br s, 1H), 7.55 (d, J = 8.4 Hz, 1H), 4.07 (s, 3H), 2.28 (s, 3H). |
| I-226 | 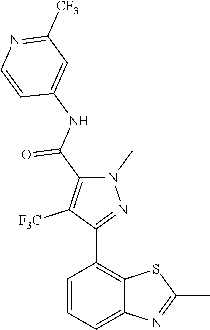 [SMILES: Cc1nc2cccc(-c3nn(C)c(C(=O)Nc4ccnc(C(F)(F)F)c4)c3C(F)(F)F)c2s1] | 2-methyl-7- (4,4,5,5- tetramethyl- 1,3,2- dioxaborolan- 2- yl)benzo[d]thi- azole | (ES, m/z): [M + H]+ 486.2, ¹H NMR (400 MHz, DMSO) 11.99 (s, 1H), 8.77-8.74 (m, 1H), 8.18 (s, 1H), 8.02 (dd, J = 0.7, 8.0 Hz, 1H), 7.90- 7.87 (m, 1H), 7.65 (dd, J = 7.7, 7.7 Hz, 1H), 7.58 (d, J = 7.6 Hz, 1H), 4.05 (s, 3H), 2.82 (s, 3H). |
| I-230 | 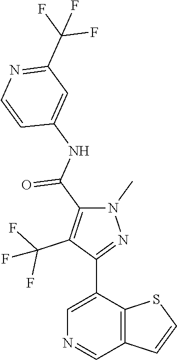 [SMILES: Cn1nc(-c2cncc3ccsc23)c(C(F)(F)F)c1C(=O)Nc1ccnc(C(F)(F)F)c1] | 7- bromothieno[3,2- c]pyridine | (ES, m/z): [M + H]+ 472.2, ¹H NMR (400 MHz, DMSO) 12.01 (s, 1H), 9.25 (s, 1H), 8.74 (d, J = 3.3 Hz, 1H), 8.56 (s, 1H), 8.19 (s, 1H), 8.00 (d, J = 5.6 Hz, 1H), 7.88 (br. s, 1H), 7.74 (d, J = 5.6 Hz, 1H), 4.09 (s, 3H). |
| I-233 | 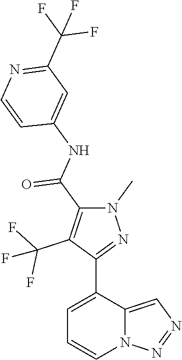 [SMILES: Cn1nc(-c2cccn3nncc23)c(C(F)(F)F)c1C(=O)Nc1ccnc(C(F)(F)F)c1] | 4-bromo- [1,2,3 ]triazolo[1,5- a]pyridine | (ES, m/z): [M + H]+ 456.0, ¹H NMR (400 MHz, DMSO) 11.99 (s, 1H), 9.21 (d, J = 7.1 Hz, 1H), 8.76 (d, J = 5.1 Hz, 1H), 8.38-8.37 (m, 1H), 8.20-8.18 (m, 1H), 7.89 (d, J = 4.0 Hz, 1H), 7.57 (d, J = 6.8 Hz, 1H), 7.35 (t, J = 7.1 Hz, 1H), 4.09 (s, 3H). |
| I-234 | 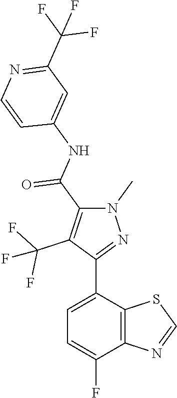 [SMILES: Cn1nc(-c2ccc(F)c3ncsc23)c(C(F)(F)F)c1C(=O)Nc1ccnc(C(F)(F)F)c1] | 4-fluoro-7- (4,4,5,5- tetramethyl- 1,3,2- dioxaborolan- 2- yl)benzo[d]thi- azole | (ES, m/z): [M + H]⁺ 490.2, ¹H NMR (400 MHz, DMSO) 11.99 (s, 1H), 9.52 (s, 1H), 8.75-8.72 (m, 1H), 8.17 (s, 1H), 7.87-7.86 (m, 1H), 7.69 (dd, J = 4.4, 8.2 Hz, 1H), 7.61 (dd, J = 8.3, 10.4 Hz, 1H), 4.06 (s, 3H). |
| I-237 | 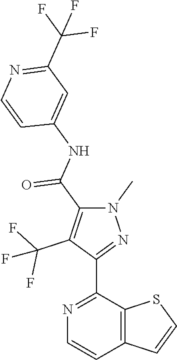 [SMILES: Cn1nc(-c2nccc3ccsc23)c(C(F)(F)F)c1C(=O)Nc1ccnc(C(F)(F)F)c1] | 7- bromothieno[2,3- c]pyridine | (ES, m/z): [M + H]+ 472.2, ¹H NMR (400 MHz, DMSO) 11.99 (s, 1H), 8.76 (s, 1H), 8.61 (d, J = 5.3 Hz, 1H), 8.20 (d, J = 5.5 Hz, 2H), 7.95 (d, J = 5.3 Hz, 1H), 7.88 (s, 1H), 7.65 (d, J = 5.4 Hz, 1H), 4.09 (s, 3H). |
| I-240 | 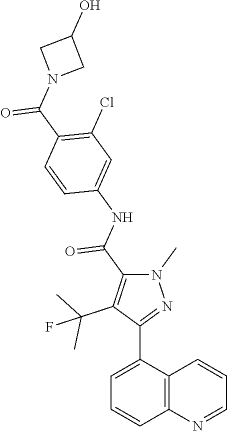 | quinolin-5- ylboronic acid | (ES, m/z): [M + H]+ 530.5, ¹H NMR (400 MHz, DMSO) 11.46 (s, 1H), 8.98 (dd, J = 1.6, 4.2 Hz, 1H), 8.21- 8.16 (m, 2H), 7.95 (d, J = 1.8 Hz, 1H), 7.89 (dd, J = 7.2, 8.5 Hz, 1H), 7.65 (d, J = 7.1 Hz, 2H), 7.60 (dd, J = 4.2, 8.6 Hz, 1H), 7.48 (d, J = 8.3 Hz, 1H), 5.81 (d, J = 6.1 Hz, 1H), 4.55- 4.48 (m, 1H), 4.24 (dd, J = 6.9, 9.7 Hz, 1H), 4.10- 4.04 (m, 4H), 3.80-3.69 (m, 2H). |
| I-244 | 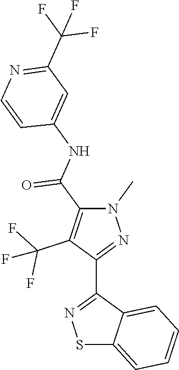 [SMILES: Cn1nc(-c2nsc3ccccc23)c(C(F)(F)F)c1C(=O)Nc1ccnc(C(F)(F)F)c1] | 3- bromobenzo[d] isothiazole | (ES, m/z): [M + H]+ 472.6, ¹H NMR (400 MHz, DMSO) 12.01 (s, 1H), 8.76 (d, J = 5.1 Hz, 1H), 8.66 (d, J = 8.2 Hz, 1H), 8.34 (d, J = 8.2 Hz, 1H), 8.20 (s, 1H), 7.89 (d, J = 4.4 Hz, 1H), 7.74-7.70 (m, 1H), 7.66-7.62 (m, 1H), 4.11 (s, 3H). |
| I-249 | 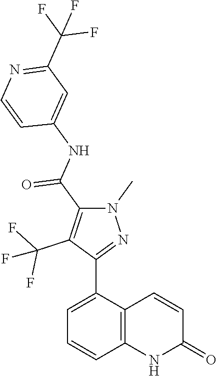 [SMILES: Cn1nc(-c2cccc3[nH]c(=O)ccc23)c(C(F)(F)F)c1C(=O)Nc1ccnc(C(F)(F)F)c1] | 5-(4,4,5,5- tetramethyl- 1,3,2- dioxaborolan- 2-yl)quinolin- 2(1H)-one | (ES, m/z): [M −H]− 480.2, ¹H NMR (400 MHz, DMSO) 11.96 (s, 1H), 11.90 (s, 1H), 8.76 (d, J = 5.1 Hz, 1H), 8.19 (s, 1H), 7.90 (d, J = 3.8 Hz, 1H), 7.68 (d, J = 9.9 Hz, 1H), 7.61 (dd, J = 7.8, 7.8 Hz, 1H), 7.46 (d, J = 8.3 Hz, 1H), 7.18 (d, J = 7.3 Hz, 1H), 6.56 (dd, J = 1.8, 9.9 Hz, 1H), 4.04 (s, 3H). |
| I-251 | 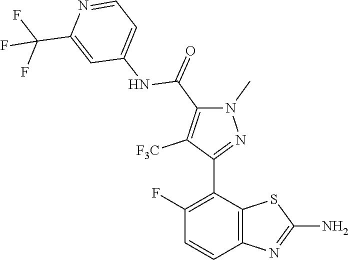 [SMILES: Cn1nc(-c2c(F)ccc3nc(N)sc23)c(C(F)(F)F)c1C(=O)Nc1ccnc(C(F)(F)F)c1] | 7-bromo-6- fluorobenzo[d] thiazol-2- amine- | (ES, m/z): [M + H]+ 505.2, ¹H NMR (400 MHz, DMSO) 11.99 (s, 1H), 8.78 (s, 1H), 8.22 (s, 1H), 7.93 (s, 1H), 7.57 (s, 2H), 7.48-7.43 (m, 1H), 7.27-7.22 (m, 1H), 4.09 (s, 3H). |
| I-280 | 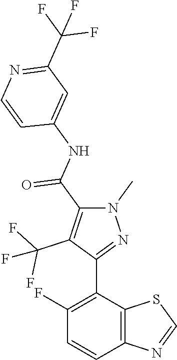 [SMILES: Cn1nc(-c2c(F)ccc3ncsc23)c(C(F)(F)F)c1C(=O)Nc1ccnc(C(F)(F)F)c1] | 6-fluoro-7- (4,4,5,5- tetramethyl- 1,3,2- dioxaborolan- 2- yl)benzo[d]thi- azole | (ES, m/z): [M + H]+ 490.2, ¹H NMR (400 MHz, DMSO) 11.97 (s, 1H), 9.44 (s, 1H), 8.76-8.74 (m, 1H), 8.29- 8.24 (m, 1H), 8.19 (s, 1H), 7.91 (s, 1H), 7.63 (dd, J = 9.5, 9.5 Hz, 1H), 4.09 (s, 3H). |
| I-281 | 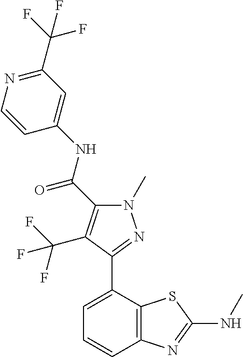 [SMILES: CNc1nc2cccc(-c3nn(C)c(C(=O)Nc4ccnc(C(F)(F)F)c4)c3C(F)(F)F)c2s1] | N-methyl-7- (4,4,5,5- tetramethyl- 1,3,2- dioxaborolan- 2- yl)benzo[d]thi- azol-2-amine | (ES, m/z): [M + H]+ 501.2, ¹H NMR (400 MHz, DMSO) 11.95 (s, 1H), 8.78-8.67 (m, 1H), 8.20-7.93 (m, 3H), 7.47-7.45 (m, 1H), 7.36 (dd, J = 8.3, 8.3 Hz, 1H), 7.20- 7.18 (m, 1H), 3.98 (s, 3H), 2.95 (d, J = 4.8 Hz, 3H). |

### Example 4—Synthesis of 3-(isoquinolin-1-yl)-1-methyl-4-(trifluoromethyl)-N-(2-(trifluoromethyl)pyridin-4-yl)-1H-pyrazole-5-carboxamide (I-105)

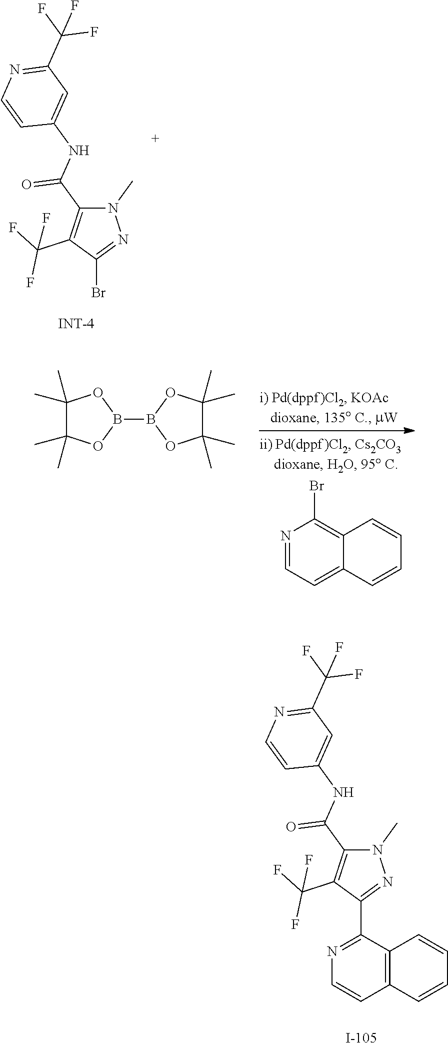

Pd(dppf)Cl₂ (14 mg, 0.0192 mmol, 0.100 eq) was added to a stirred degassed mixture of bis(pinacolato)diboron (54 mg, 0.211 mmol, 1.10 eq), 5-bromo-2-methyl-4-(trifluoromethyl)-N-[2-(trifluoromethyl)-4-pyridyl]pyrazole-3-carboxamide (80 mg, 0.192 mmol, 1.00 eq) and KOAc (56 mg, 0.575 mmol, 3.00 eq) in 1,4-dioxane (3 mL), the reaction mixture was heated at 135° C. in the microwave for 4 hours. 1-Bromoisoquinoline (40 mg, 0.192 mmol, 1.00 eq), Cs₂CO₃ (187 mg, 0.575 mmol, 3.00 eq), Pd(dppf)Cl₂ (14 mg, 0.0192 mmol, 0.100 eq) and water (0.3 mL) were added and the reaction mixture was heated at 95° C. for 16 hours. The reaction was cooled to rt and diluted with EtOAc. The organic phase was washed with saturated aqueous NaHCO₃ and brine. The organics were dried and concentrated, the crude residue was purified by column chromatography eluting with 0-100% EtOAc in cyclohexane followed by reverse phase HPLC to give the title compound as an off-white solid (11.4 mg, 0.025 mmol, 13%). (ES, m/z): [M+H]⁺ 466.2, ¹H NMR (400 MHz, DMSO) δ 11.96 (s, 1H), 8.75-8.72 (m, 1H), 8.64 (d, J=5.6 Hz, 1H), 8.22 (d, J=6.8 Hz, 2H), 8.10 (d, J=8.3 Hz, 1H), 7.97 (d, J=5.6 Hz, 1H), 7.90-7.83 (m, 2H), 7.73 (dd, J=7.3, 7.3 Hz, 1H), 4.09 (s, 3H).

### Example 5—Synthesis of Additional Compounds

Compounds in Table 5 were prepared using a procedure analagous to that described in Example 4.

TABLE 5

| Compound | | Starting | |
|---|---|---|---|
| No. | Structure | Materials | Analytical Data |
| I-106 | 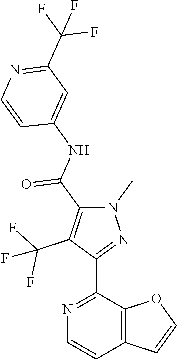 [SMILES: Cn1nc(-c2nccc3ccoc23)c(C(F)(F)F)c1C(=O)Nc1ccnc(C(F)(F)F)c1] | 7- chlorofuro [2,3-c]pyridine | (ES, m/z): [M + H]⁺ 456.2, ¹H NMR (400 MHz, DMSO) δ 12.03-11.95 (m, 1H), 8.73-8.73 (m, 1H), 8.49 (d, J = 5.1 Hz, 1H), 8.31 (d, J = 2.0 Hz, 1H), 8.19 (s, 1H), 7.90-7.87 (m, 1H), 7.82 (d, J = 5.1 Hz, 1H), 7.18 (d, J = 2.3 Hz, 1H), 4.06 (s, 3H) |
| I-107 | 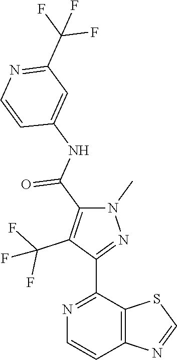 [SMILES: Cn1nc(-c2nccc3ncsc23)c(C(F)(F)F)c1C(=O)Nc1ccnc(C(F)(F)F)c1] | 4- chlorothiazolo [5,4-c]pyridine | (ES, m/z): [M + H]⁺ 473.2, ¹H NMR (400 MHz, DMSO) δ 12.06 (s, 1H), 9.83 (s, 1H), 8.84 (d, J = 5.3 Hz, 1H), 8.79 (d, J = 5.3 Hz, 1H), 8.24 (s, 1H), 8.21 (d, J = 5.5 Hz, 1H), 7.92 (d, J = 4.3 Hz, 1H), 4.14 (s, 3H) |
| I-108 | 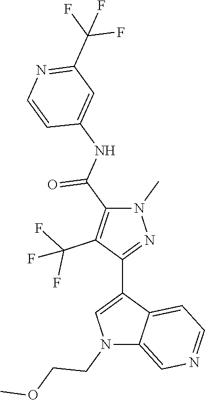 [SMILES: COCCn1cc(-c2nn(C)c(C(=O)Nc3ccnc(C(F)(F)F)c3)c2C(F)(F)F)c2ccncc21] | 3-bromo- 1-(2- methoxyethyl)- 1H- pyrrolo[2,3-c] pyridine | (ES, m/z): [M + H]⁺ 513.2, ¹H NMR (400 MHz, DMSO) δ 11.91 (s, 1H), 8.98 (s, 1H), 8.75 (d, J = 5.3 Hz, 1H), 8.26 (d, J = 5.6 Hz, 1H), 8.19 (d, J = 1.5 Hz, 1H), 7.98-7.96 (m, 1H), 7.90-7.87 (m, 1H), 7.84 (s, 1H), 4.57 (dd, J = 5.1, 5.1 Hz, 2H), 4.01 (s, 3H), 3.71 (dd, J = 5.1, 5.1 Hz, 2H), 2.33 (s, 3H) |
| I-109 | 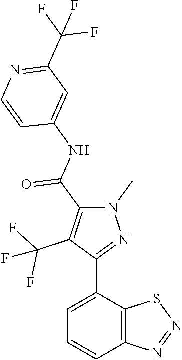 [SMILES: Cn1nc(-c2cccc3nnsc23)c(C(F)(F)F)c1C(=O)Nc1ccnc(C(F)(F)F)c1] | 7- bromobenzo[d][1,2- 3]thiadiazole | (ES, m/z): [M + H]⁺ 473 ¹H NMR (400 MHz, DMSO) δ 11.96 (br s, 1H), 8.84 (dd, J = 8.2, 0.6 Hz, 1H), 8.68 (d, J = 4.3 Hz, 1H), 8.14 (s, 1H), 8.09 (d, J = 7.4 Hz, 1H), 7.98 (t, J = 7.8 Hz, 1H), 7.81 (br s, 1H), 4.10 (s, 3H) |
| I-110 | 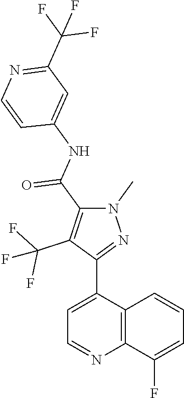 [SMILES: Cn1nc(-c2ccnc3c(F)cccc23)c(C(F)(F)F)c1C(=O)Nc1ccnc(C(F)(F)F)c1] | 4-bromo- 8- fluoroquinoline | (ES, m/z): [M + H]⁺ 484.2 ¹H NMR (400 MHz, DMSO) δ 11.96 (s, 1H), 9.10 (d, J = 4.3 Hz, 1H), 8.76 (d, J = 5.3 Hz, 1H), 8.20 (s, 1H), 7.90 (d, J = 4.3 Hz, 1H), 7.74-7.63 (m, 4H), 4.10 (s, 3H) |
| I-111 | 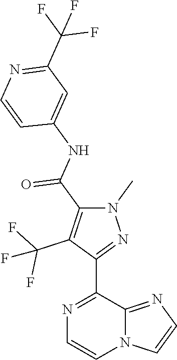 [SMILES: Cn1nc(-c2nccn3ccnc23)c(C(F)(F)F)c1C(=O)Nc1ccnc(C(F)(F)F)c1] | 8- bromoimidazo[1,2- a]pyrazine | (ES, m/z): [M + H]⁺ 456.2 ¹H NMR (400 MHz, DMSO) δ 12.01 (s, 1H), 8.76 (d, J = 5.5 Hz, 1H), 8.73 (d, J = 4.5 Hz, 1H), 8.27 (d, J = 1.0 Hz, 1H), 8.21 (d, J = 1.6 Hz, 1H), 8.02 (d, J = 4.5 Hz, 1H), 7.92-7.88 (m, 1H), 7.86 (d, J = 1.0 Hz, 1H), 4.07 (s, 3H) |
| I-112 | 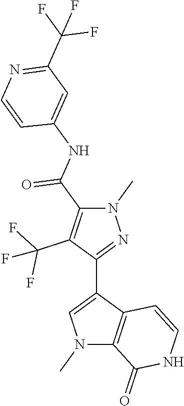 [SMILES: Cn1nc(-c2cn(C)c3c(=O)[nH]ccc23)c(C(F)(F)F)c1C(=O)Nc1ccnc(C(F)(F)F)c1] | 3-bromo- 1-methyl-1,6- dihydro-7H- pyrrolo[2,3-c] pyridin-7-one | (ES, m/z): [M − H]− 483, ¹H NMR (400 MHz, DMSO) δ 11.88 (d, J = 1.5 Hz, 1H), 11.05 (d, J = 4.0 Hz, 1H), 8.76-8.72 (m, 1H), 8.17- 8.16 (m, 1H), 7.87 (s, 1H), 7.51 (s, 1H), 6.96-6.92 (m, 1H), 6.84 (d, J = 5.8 Hz, 1H), 4.13 (s, 3H), 3.96 (s, 3H) |
| I-113 | 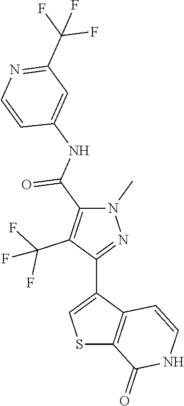 [SMILES: Cn1nc(-c2csc3c(=O)[nH]ccc23)c(C(F)(F)F)c1C(=O)Nc1ccnc(C(F)(F)F)c1] | 3- bromothieno [2,3-c]pyridin- 7(6H)-one | (ES, m/z): [M + H]⁺ 488.2, ¹H NMR (400 MHz, DMSO) δ d 11.95 (s, 1H), 11.73-11.69 (m, 1H), 8.78- 8.74 (m, 1H), 8.22-8.18 (m, 2H), 7.89 (d, J = 4.6 Hz, 1H), 7.39-7.35 (m, 1H), 6.83 (d, J = 6.9 Hz, 1H), 4.04 (s, 3H) |
| I-114 | 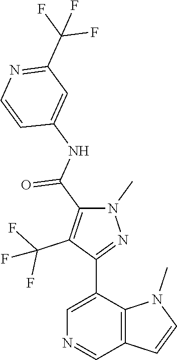 [SMILES: Cn1nc(-c2cncc3ccn(C)c23)c(C(F)(F)F)c1C(=O)Nc1ccnc(C(F)(F)F)c1] | 7-bromo- 1-methyl-1H- pyrrolo[3,2-c]pyridine | (ES, m/z): [M + H]⁺ 469.2, ¹H NMR (400 MHz, DMSO) δ 11.97 (br s, 1H), 8.92 (s, 1H), 8.76 (d, J = 5.5 Hz, 1H), 8.21-8.17 (m, 1H), 8.08 (s, 1H), 7.92 (d, J = 4.2 Hz, 1H), 7.46 (d, J = 3.2 Hz, 1H), 6.71 (d, J = 3.2 Hz, 1H), 4.06 (s, 3H), 3.44 (s, 3H) |
| I-115 | 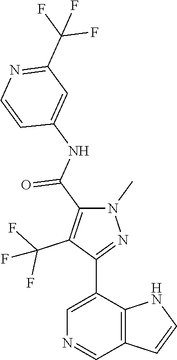 [SMILES: Cn1nc(-c2cncc3cc[nH]c23)c(C(F)(F)F)c1C(=O)Nc1ccnc(C(F)(F)F)c1] | 7-bromo- 1H- pyrrolo[3,2-c] pyridine | (ES, m/z): [M + H]⁺ 455.2, ¹H NMR (400 MHz, DMSO) δ 12.01 (br s, 1H), 11.46 (s, 1H), 8.91 (s, 1H), 8.76 (d, J = 5.5 Hz, 1H), 8.31 (s, 1H), 8.21 (d, J = 1.6 Hz, 1H), 7.90 (d, J = 3.9 Hz, 1H), 7.51 (t, J = 2.8 Hz, 1H), 6.71 (dd, J = 3.2, 1.8 Hz, 1H), 4.07 (s, 3H) |
| I-116 | 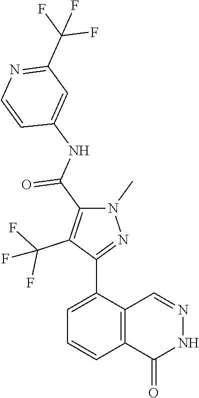 [SMILES: Cn1nc(-c2cccc3c(=O)[nH]ncc23)c(C(F)(F)F)c1C(=O)Nc1ccnc(C(F)(F)F)c1] | 5- bromophthalazin- 1(2H)-one | (ES, m/z): [M + H]⁺ 483, ¹H NMR (400 MHz, DMSO) δ 12.87 (s, 1H), 11.90-11.87 (m, 1H), 8.78-8.74 (m, 1H), 8.39 (d, J = 7.6 Hz, 1H), 8.19 (s, 1H), 8.13 (s, 1H), 8.00- 7.88 (m, 2H), 7.87 (s, 1H), 4.07 (s, 3H) |
| I-118 | 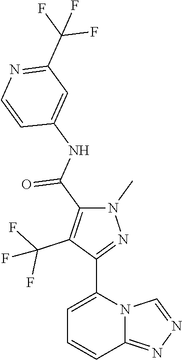 [SMILES: Cn1nc(-c2cccc3nncn23)c(C(F)(F)F)c1C(=O)Nc1ccnc(C(F)(F)F)c1] | 5-bromo- [1,2,4]triazolo [4,3-a]pyridine | (ES, m/z): [M + H]⁺ 456.2, ¹H NMR (400 MHz, DMSO) δ 12.06 (s, 1H), 9.47 (s, 1H), 8.74 (d, J = 5.6 Hz, 1H), 8.18 (s, 1H), 7.97 (d, J = 9.3 Hz, 1H), 7.86 (s, 1H), 7.56 (dd, J = 6.8, 9.3 Hz, 1H), 7.18 (d, J = 6.8 Hz, 1H), 4.11 (s, 3H) |
| I-119 | 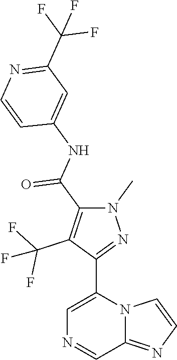 [SMILES: Cn1nc(-c2cncc3nccn23)c(C(F)(F)F)c1C(=O)Nc1ccnc(C(F)(F)F)c1] | 5- bromoimidazo [1,2-a]pyrazine | (ES, m/z): [M + H]⁺ 456.4, ¹H NMR (400 MHz, DMSO) δ 12.09 (s, 1H), 9.22 (s, 1H), 8.70 (s, 1H), 8.22 (s, 1H), 8.17 (s, 1H), 8.03 (s, 1H), 7.97 (d, J = 1.0 Hz, 1H), 7.82 (s, 1H), 4.12 (s, 3H) |

### Example 6—Synthesis of N-(6-(2H-1,2,3-triazol-2-yl)-5-(trifluoromethyl)pyridin-3-yl)-1-methyl-3-(thieno[2,3-c]pyridin-3-yl)-4-(trifluoromethyl)-1H-pyrazole-5-carboxamide (I-120)

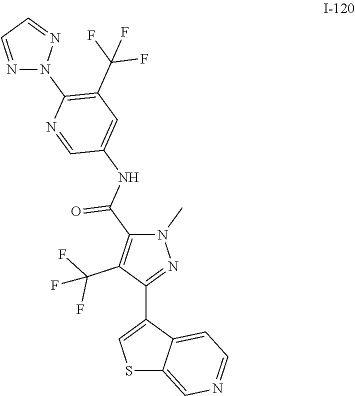 [SMILES: Cn1nc(-c2csc3cnccc23)c(C(F)(F)F)c1C(=O)Nc1cnc(-n2nccn2)c(C(F)(F)F)c1]

### Step 1: Synthesis of 3-(4,4,5,5-tetramethyl-1,3,2-dioxaborolan-2-yl) thieno[2,3-c]pyridine (INT 6)

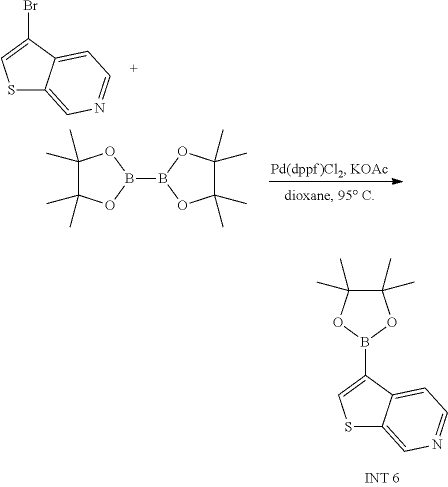

The compound was synthesized following based on procedures described in Example 2 and isolated as a white solid (120 mg, 0.46 mmol, 28%). ¹H NMR (400 MHz, CDCl₃) δ 9.17 (s, 1H), 8.53 (d, J=5.4 Hz, 1H), 8.29 (s, 1H), 8.22 (d, J=5.5 Hz, 1H), 1.39 (s, 12H).

### Step 2: Synthesis of ethyl 1-methyl-3-(thieno[2,3-c]pyridin-3-yl)-4-(trifluoromethyl)-1H-pyrazole-5-carboxylate (INT 7)

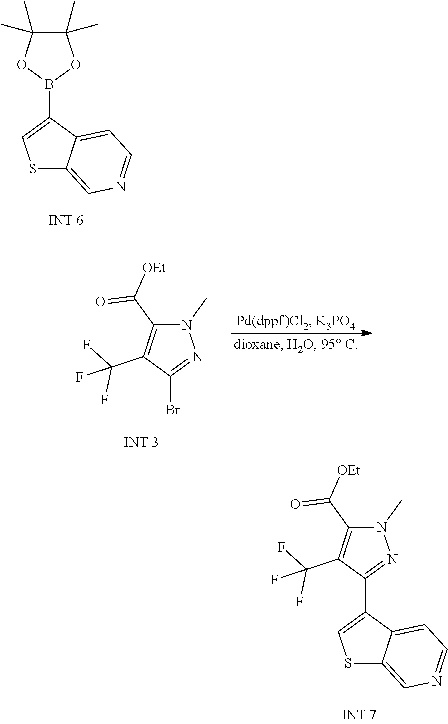 [SMILES: CC1(C)OB(c2csc3cnccc23)OC1(C)C ; CCOC(=O)c1c(C(F)(F)F)c(Br)nn1C ; CCOC(=O)c1c(C(F)(F)F)c(-c2csc3cnccc23)nn1C]

The title compound was synthesized from 3-(4,4,5,5-tetramethyl-1,3,2-dioxaborolan-2-yl) thieno[2,3-c]pyridine INT 6 and ethyl 3-bromo-1-methyl-4-(trifluoromethyl)-1H-pyrazole-5-carboxylate INT 3 based on procedures described in Example 2 and isolated by column chromatography as an off-white solid (100 mg, 0.28 mmol, 42%). (ES, m/z): [M+H]⁺ 355.0 ¹H NMR (400 MHz, DMSO) 9.35 (s, 1H), 8.52 (d, J=5.5 Hz, 1H), 8.29 (s, 1H), 7.72 (d, J=5.5 Hz, 1H), 4.44 (q, J=7.1 Hz, 2H), 4.17 (s, 3H), 1.35 (t, J=7.1 Hz, 3H).

### Step 3: Synthesis of N-(6-(2H-1,2,3-triazol-2-yl)-5-(trifluoromethyl)pyridin-3-yl)-1-methyl-3-(thieno[2,3-c]pyridin-3-yl)-4-(trifluoromethyl)-1H-pyrazole-5-carboxamide (I-120)

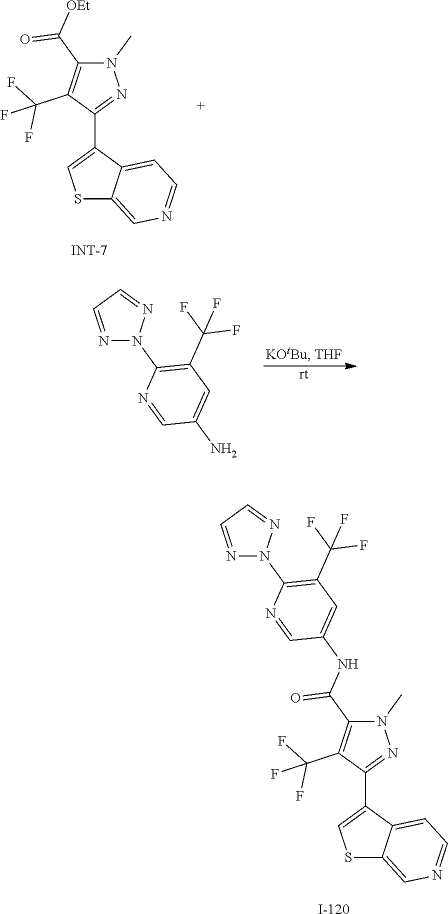 [SMILES: CCOC(=O)c1c(C(F)(F)F)c(-c2csc3cnccc23)nn1C ; "INT-7" ; Nc1cnc(-n2nccn2)c(C(F)(F)F)c1 ; Cn1nc(-c2csc3cnccc23)c(C(F)(F)F)c1C(=O)Nc1cnc(-n2nccn2)c(C(F)(F)F)c1 ; "I-120"]

The title compound was synthesized from INT 7 and 6-(2H-1,2,3-triazol-2-yl)-5-(trifluoromethyl)pyridine-3-amine based on procedures described in Example 2 and isolated by HPLC as an off-white solid (22.6 mg, 0.042 mmol, 18%). (ES, m/z): [M+H]⁺ 539.2, ¹H NMR (400 MHz, DMSO) 12.10 (s, 1H), 9.37 (s, 1H), 9.08-9.06 (m, 1H), 8.86 (d, J=2.3 Hz, 1H), 8.58 (d, J=5.6 Hz, 1H), 8.34 (s, 1H), 8.21 (s, 2H), 8.04 (d, J=5.6 Hz, 1H), 4.11 (s, 3H).

### Example 7—Synthesis of Additional Compounds

Compounds in Table 6 were prepared using a procedure analogous to that described in Example 6.

TABLE 6

| Compound | | Starting | |
|---|---|---|---|
| No. | Structure | Materials | Analytical Data |
| I-121 | 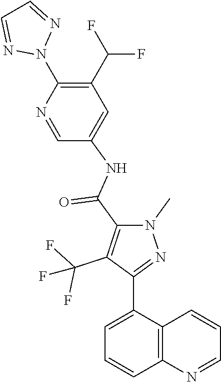 [SMILES: Cn1nc(-c2cccc3ncccc23)c(C(F)(F)F)c1C(=O)Nc1cnc(-n2nccn2)c(C(F)F)c1] | ethyl 1-methyl-3- (quinolin-5-yl)-4- (trifluoromethyl)-1H- pyrazole-5-carboxylate and 5-(difluoromethyl)- 6-(2H-1,2,3-triazol-2- yl)pyridin-3-amine | (ES, m/z): [M + H]⁺ 515.2, ¹H NMR (400 MHz, DMSO) δ 11.90 (s, 1H), 9.01-8.98 (m, 2H), 8.76 (d, J = 2.3 Hz, 1H), 8.25 (s, 2H), 8.20 (s, 1H), 8.18 (s, 1H), 7.92-7.87 (m, 1H), 7.67 (d, J = 6.8 Hz, 1H), 7.61 (dd, J = 4.0, 8.6 Hz, 1H), 7.43 (t, J = 54.3 Hz, 1H), 4.11 (s, 3H) |
| I-122 | 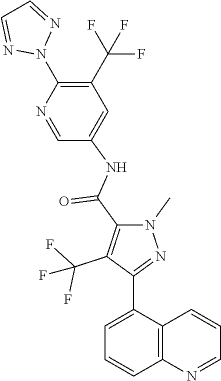 [SMILES: Cn1nc(-c2cccc3ncccc23)c(C(F)(F)F)c1C(=O)Nc1cnc(-n2nccn2)c(C(F)(F)F)c1] | ethyl 1-methyl-3- (quinolin-5-yl)-4- (trifluoromethyl)-1H- pyrazole-5-carboxylate and 6-(2H-1,2,3-triazol- 2-yl)-5- (trifluoromethyl)pyridin- 3-amine | ES, m/z): [M + H]⁺ 533.2, ¹H NMR (400 MHz, DMSO) δ 12.09 (br s, 1H), 9.08 (br s, 1H), 9.00 (dd, J = 1.6, 4.1 Hz, 1H), 8.89 (d, J = 2.4 Hz, 1H), 8.22 (s, 2H), 8.20 (s, 1H), 8.18 (s, 1H), 7.90 (dd, J = 7.1, 8.5 Hz, 1H), 7.68 (d, J = 7.0 Hz, 1H), 7.62 (dd, J = 4.0, 8.5 Hz, 1H), 4.12 (s, 3H) |
| I-124 | 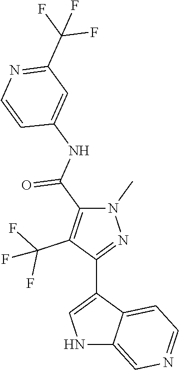 [SMILES: Cn1nc(-c2c[nH]c3cnccc23)c(C(F)(F)F)c1C(=O)Nc1ccnc(C(F)(F)F)c1] | tert-butyl 3-(5- (ethoxycarbonyl)-1- methyl-4- (trifluoromethyl)-1H- pyrazol-3-yl)-1H- pyrrolo[2,3-c]pyridine- 1-carboxylate and 2- (trifluoromethyl)pyridin- 4-amine | (ES, m/z): [M + H]⁺ 454.3, ¹H NMR (400 MHz, DMSO) δ 12.00 (m, 2H), 8.85 (s, 1H), 8.75 (d, J = 5.5 Hz, 1H), 8.23 (d, J = 5.5 Hz, 1H), 8.19 (s, 1H), 7.97 (d, J = 5.5 Hz, 1H), 7.88 (dd, J = 1.7, 5.5 Hz, 1H), 7.83 (s, 1H), 4.01 (s, 3H) |
| I-125 | 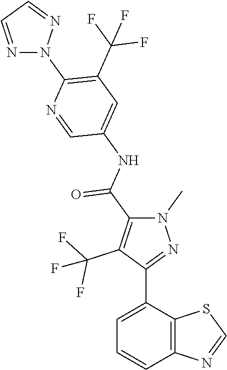 [SMILES: Cn1nc(-c2cccc3ncsc23)c(C(F)(F)F)c1C(=O)Nc1cnc(-n2nccn2)c(C(F)(F)F)c1] | ethyl 3-(benzo[d]thiazol- 7-yl)-1-methyl-4- (trifluoromethyl)-1H- pyrazole-5-carboxylate and 6-(2H-1,2,3-triazol- 2-yl)-5- (trifluoromethyl)pyridin- 3-amine | (ES, m/z): [M + H]⁺ 538.4, H NMR (400 MHz, DMSO) δ 12.19 (s, 1H), 9.55 (s, 1H), 9.15 (s, 1H), 8.91 (d, J = 2.4 Hz, 1H), 8.31-8.24 (m, 3H), 7.83-7.73 (m, 2H), 4.17 (s, 3H) |
| I-126 | 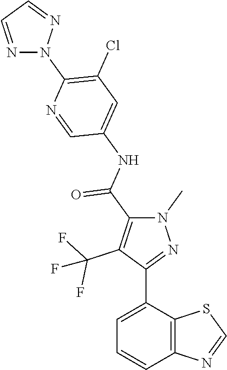 [SMILES: Cn1nc(-c2cccc3ncsc23)c(C(F)(F)F)c1C(=O)Nc1cnc(-n2nccn2)c(Cl)c1] | ethyl 3-(benzo[d]thiazol- 7-yl)-1-methyl-4- (trifluoromethyl)-1H- pyrazole-5-carboxylate and 5-chloro-6-(2H- 1,2,3-triazol-2- yl)pyridin-3-amine | (ES, m/z): [M + H]⁺ 505.2, ¹H NMR (400 MHz, CDCl₃) δ 12.05 (s, 1H), 9.54 (s, 1H), 8.85 (s, 1H), 8.69 (d, J = 2.2 Hz, 1H), 8.27 (dd, J = 7.8, 1.2 Hz, 1H), 8.26 (s, 2H), 7.82-7.73 (m, 2H), 4.15 (s, 3H) |
| I-127 | 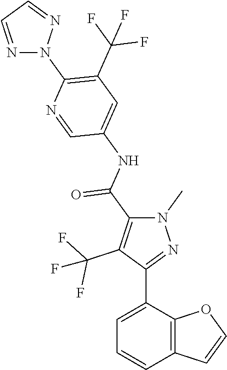 [SMILES: Cn1nc(-c2cccc3ccoc23)c(C(F)(F)F)c1C(=O)Nc1cnc(-n2nccn2)c(C(F)(F)F)c1] | ethyl 3-(benzofuran-7- yl)-1-methyl-4- (trifluoromethyl)-1H- pyrazole-5-carboxylate and 6-(2H-1,2,3-triazol- 2-yl)-5- (trifluoromethyl)pyridin- 3-amine | (ES, m/z): [M + H]⁺ 522.3, ¹H NMR (400 MHz, CDCl₃) δ 12.00 (s, 1H), 9.10 (br s, 1H), 8.87 (d, J = 2.3 Hz, 1H), 8.22 (s, 2H), 8.05 (d, J = 2.2 Hz, 1H), 7.80 (t, J = 4.5 Hz, 1H), 7.41-7.36 (m, 2H), 7.07 (d, J = 2.2 Hz, 1H), 4.09 (s, 3H) |
| I-128 | 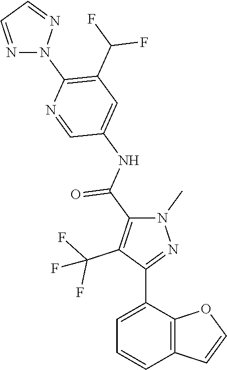 [SMILES: Cn1nc(-c2cccc3ccoc23)c(C(F)(F)F)c1C(=O)Nc1cnc(-n2nccn2)c(C(F)F)c1] | ethyl 3-(benzofuran-7- yl)-1-methyl-4- (trifluoromethyl)-1H- pyrazole-5-carboxylate and 5-(difluoromethyl)- 6-(2H-1,2,3-triazol-2- yl)pyridin-3-amine | (ES, m/z): [M + H]⁺ 504.3 ¹H NMR (400 MHz, CDCl₃) δ 11.92 (s, 1H), 8.99 (br s, 1H), 8.75 (d, J = 2.3 Hz, 1H), 8.24 (s, 2H), 8.05 (d, J = 2.2 Hz, 1H), 7.80 (t, J = 4.5 Hz, 1H), 7.42 (t, J = 54.2 Hz, 1H), 7.38 (d, J = 5.0 Hz, 2H), 7.07 (d, J = 2.2 Hz, 1H), 4.08 (s, 3H) |
| I-129 | 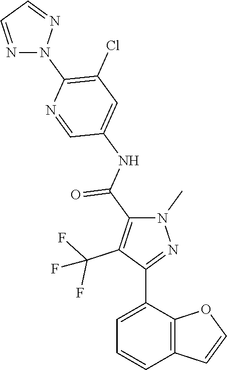 [SMILES: Cn1nc(-c2cccc3ccoc23)c(C(F)(F)F)c1C(=O)Nc1cnc(-n2nccn2)c(Cl)c1] | ethyl 3-(benzofuran-7- yl)-1-methyl-4- (trifluoromethyl)-1H- pyrazole-5-carboxylate and 5-chloro-6-(2H- 1,2,3-triazol-2- yl)pyridin-3-amine | (ES, m/z): [M + H]⁺ 488.2 ¹H NMR (400 MHz, CDCl₃) δ 11.96 (s, 1H), 8.82 (d, J = 2.2 Hz, 1H), 8.64 (d, J = 2.2 Hz, 1H), 8.20 (s, 2H), 8.05 (d, J = 2.2 Hz, 1H), 7.80 (t, J = 4.5 Hz, 1H), 7.38 (d, J = 4.8 Hz, 2H), 7.07 (d, J = 2.2 Hz, 1H), 4.08 (s, 3H) |
| I-130 | 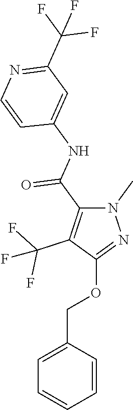 [SMILES: Cn1nc(OCc2ccccc2)c(C(F)(F)F)c1C(=O)Nc1ccnc(C(F)(F)F)c1] | methyl 3-(benzyloxy)-1- methyl-4- (trifluoromethyl)-1H- pyrazole-5-carboxylate and 2- (trifluoromethyl)pyridin- 4-amine | (ES, m/z): [M + H]⁺ 445.2, ¹H NMR (400 MHz, DMSO) δ 11.78 (s, 1H), 8.70 (d, J = 4.8 Hz, 1H), 8.13 (s, 1H), 7.82 (d, J = 5.3 Hz, 1H), 7.47-7.34 (m, 5H), 5.33 (s, 2H), 3.78 (s, 3H) |
| I-131 | 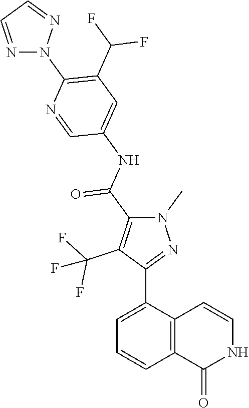 [SMILES: Cn1nc(-c2cccc3c(=O)[nH]ccc23)c(C(F)(F)F)c1C(=O)Nc1cnc(-n2nccn2)c(C(F)F)c1] | ethyl 1-methyl-3-(1-oxo- 1,2-dihydroisoquinolin- 5-yl)-4- (trifluoromethyl)-1H- pyrazole-5-carboxylate and 5-(difluoromethyl)- 6-(2H-1,2,3-triazol-2- yl)pyridin-3-amine | (ES, m/z): [M + H]⁺ 531.2, ¹H NMR (400 MHz, DMSO) δ 11.89 (s, 1H), 11.45-11.42 (m, 1H), 8.98 (s, 1H), 8.75 (d, J = 2.0 Hz, 1H), 8.36-8.33 (m, 1H), 8.25- 8.24 (m, 2H), 7.71- 7.68 (m, 1H), 7.62-7.57 (m, 1H), 7.42 (t, J = 54.4 Hz, 1H), 7.25-7.21 (m, 1H), 6.30 (d, J = 7.1 Hz, 1H), 4.08 (s, 3H) |
| I-132 | 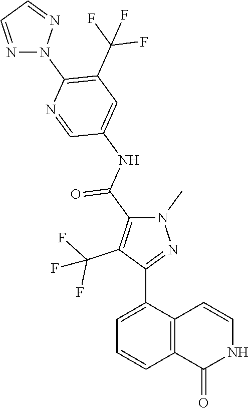 [SMILES: Cn1nc(-c2cccc3c(=O)[nH]ccc23)c(C(F)(F)F)c1C(=O)Nc1cnc(-n2nccn2)c(C(F)(F)F)c1] | ethyl 1-methyl-3-(1-oxo- 1,2-dihydroisoquinolin- 5-yl)-4- (trifluoromethyl)-1H- pyrazole-5-carboxylate and 6-(2H-1,2,3-triazol- 2-yl)-5- (trifluoromethyl)pyridin- 3-amine | (ES, m/z): [M + H]⁺ 549.3, ¹H NMR (400 MHz, DMSO) δ 12.05 (s, 1H), 11.44 (d, J = 5.8 Hz, 1H), 9.09 (s, 1H), 8.86 (d, J = 2.5 Hz, 1H), 8.36-8.33 (m, 1H), 8.22 (s, 2H), 7.70 (d, J = 7.1 Hz, 1H), 7.60 (t, J = 7.7 Hz, 1H), 7.22 (dd, J = 6.1, 7.3 Hz, 1H), 6.31-6.28 (m, 1H), 4.09 (s, 3H) |
| I-133 | 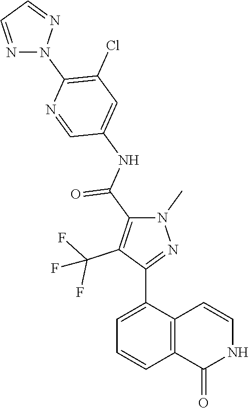 [SMILES: Cn1nc(-c2cccc3c(=O)[nH]ccc23)c(C(F)(F)F)c1C(=O)Nc1cnc(-n2nccn2)c(Cl)c1] | ethyl 1-methyl-3-(1-oxo- 1,2-dihydroisoquinolin- 5-yl)-4- (trifluoromethyl)-1H- pyrazole-5-carboxylate and 5-chloro-6-(2H- 1,2,3-triazol-2- yl)pyridin-2-amine | (ES, m/z): [M + H]⁺ 515.2, ¹H NMR (400 MHz, DMSO) δ 11.93 (s, 1H), 11.48-11.44 (m, 1H), 8.81 (d, J = 2.1 Hz, 1H), 8.66 (d, J = 2.3 Hz, 1H), 8.35 (d, J = 7.7 Hz, 1H), 8.21-8.21 (m, 2H), 7.71-7.68 (m, 1H), 7.60 (t, J = 7.7 Hz, 1H), 7.23 (dd, J = 5.8, 7.3 Hz, 1H), 6.30 (d, J = 7.3 Hz, 1H), 4.09-4.07 (m, 3H) |
| I-134 | 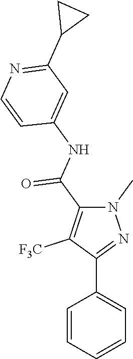 [SMILES: Cn1nc(-c2ccccc2)c(C(F)(F)F)c1C(=O)Nc1ccnc(C2CC2)c1] | ethyl 1-methyl-3-phenyl- 4-(trifluoromethyl)-1H- pyrazole-5-carboxylate and 2- cyclopropylpyridin-4- amine | (ES, m/z): [M + H]⁺ 387.2, ¹H NMR (400 MHz, DMSO) δ 11.40 (s, 1H), 8.35 (d, J = 5.6 Hz, 1H), 7.61-7.57 (m, 3H), 7.55-7.48 (m, 3H), 7.38 (dd, J = 2.0, 5.6 Hz, 1H), 3.95 (s, 3H), 2.12-2.04 (m, 1H), 0.96- 0.90 (m, 4H) |
| I-135 | 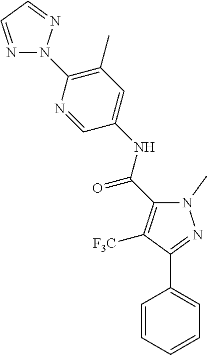 | ethyl 1-methyl-3-phenyl- 4-(trifluoromethyl)-1H- pyrazole-5-carboxylate and 5-methyl-6-(2H- 1,2,3-triazol-2- yl)pyridin-3-amine | (ES, m/z): [M + H]⁺ 482.2, ¹H NMR (400 MHz, DMSO) δ 11.63 (s, 1H), 8.69 (d, J = 2.3 Hz, 1H), 8.31 (d, J = 2.0 Hz, 1H), 8.14 (s, 2H), 7.61 (dd, J = 1.6, 7.9 Hz, 2H), 7.55-7.48 (m, 3H), 4.00 (s, 3H), 2.26 (s, 3H) |
| I-136 | 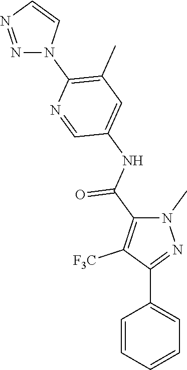 [SMILES: Cc1cc(NC(=O)c2c(C(F)(F)F)c(-c3ccccc3)nn2C)cnc1-n1ccnn1] | ethyl 1-methyl-3-phenyl- 4-(trifluoromethyl)-1H- pyrazole-5-carboxylate and 5-methyl-6-(1H- 1,2,3-triazol-1- yl)pyridin-3-amine | (ES, m/z): [M + H]⁺ 428.2, ¹H NMR (400 MHz, DMSO) δ 11.67 (s, 1H), 8.72 (d, J = 2.4 Hz, 1H), 8.61 (d, J = 1.1 Hz, 1H), 8.33 (d, J = 1.9 Hz, 1H), 7.99 (d, J = 1.1 Hz, 1H), 7.61 (dd, J = 1.4, 7.9 Hz, 2H), 7.56-7.49 (m, 3H), 4.01 (s, 3H), 2.38 (s, 3H) |
| I-137 | 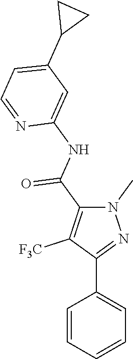 [SMILES: Cn1nc(-c2ccccc2)c(C(F)(F)F)c1C(=O)Nc1cc(C2CC2)ccn1] | ethyl 1-methyl-3-phenyl- 4-(trifluoromethyl)-1H- pyrazole-5-carboxylate and 4- cyclopropylpyridin-2- amine | (ES, m/z): [M + H]⁺ 387.2, ¹H NMR (400 MHz, DMSO) δ 11.47 (s, 1H), 8.22-8.18 (m, 1H), 7.95-7.92 (m, 1H), 7.59 (d, J = 6.8 Hz, 2H), 7.54-7.46 (m, 3H), 6.92 (d, J = 4.3 Hz, 1H), 3.93 (s, 3H), 2.05- 1.98 (m, 1H), 1.14-1.07 (m, 2H), 0.84-0.78 (m, 2H) |
| I-138 | 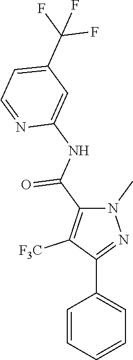 [SMILES: Cn1nc(-c2ccccc2)c(C(F)(F)F)c1C(=O)Nc1cc(C(F)(F)F)ccn1] | ethyl 1-methyl-3-phenyl- 4-(trifluoromethyl)-1H- pyrazole-5-carboxylate and 4- (trifluoromethyl)pyridin- 2-amine | (ES, m/z): [M + H]⁺ 415.2, ¹H NMR (400 MHz, DMSO) δ 12.09 (s, 1H), 8.71 (d, J = 5.0 Hz, 1H), 8.50-8.46 (m, 1H), 7.65 (d, J = 4.8 Hz, 1H), 7.60 (dd, J = 1.4, 7.6 Hz, 2H), 7.55-7.48 (m, 3H), 3.97 (s, 3H) |
| I-139 | 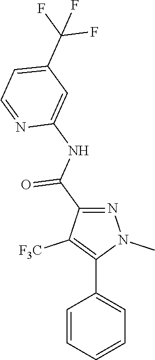 [SMILES: Cn1nc(C(=O)Nc2cc(C(F)(F)F)ccn2)c(C(F)(F)F)c1-c1ccccc1] | ethyl 1-methyl-5-phenyl- 4-(trifluoromethyl)-1H- pyrazole-3-carboxylate and 4- (trifluoromethyl)pyridin- 2-amine | (ES, m/z): [M + H]⁺ 415.2, ¹H NMR (400 MHz, DMSO) δ 9.59- 9.55 (m, 1H), 8.71 (s, 1H), 8.50 (d, J = 5.1 Hz, 1H), 7.55-7.52 (m, 3H), 7.38-7.34 (m, 2H), 7.29 (dd, J = 0.9, 5.1 Hz, 1H), 3.75-3.74 (m, 3H) |
| I-206 | 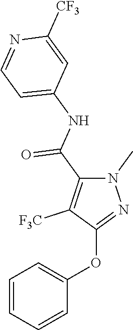 [SMILES: Cn1nc(Oc2ccccc2)c(C(F)(F)F)c1C(=O)Nc1ccnc(C(F)(F)F)c1] | methyl 1-methyl-3- phenoxy-4- (trifluoromethyl)-1H- pyrazole-5-carboxylate and 2- (trifluoromethyl)pyridin- 4-amine | (ES, m/z): [M + H]+ 431.2, ¹H NMR (400 MHz, DMSO) d 11.85 (s, 1H), 8.74 (d, J = 5.3 Hz, 1H), 8.15 (s, 1H), 7.85 (d, J = 5.1 Hz, 1H), 7.45-7.40 (m, 2H), 7.23- 7.15 (m, 3H), 3.83 (s, 3H). |
| I-208 | 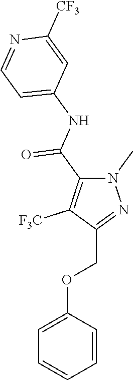 [SMILES: Cn1nc(COc2ccccc2)c(C(F)(F)F)c1C(=O)Nc1ccnc(C(F)(F)F)c1] | Potassium phenoxymethyl- trifluoroborate | (ES, m/z): [M + H]+ 445.2, 1H NMR (400 MHz, DMSO-d6) δ 11.86 (br s, 1H), 8.67 (br s, 1H), 8.13 (s, 1H), 7.81 (br s, 1H), 7.31 (dd, J = 8.6, 7.5 Hz, 2H), 7.03 (d, J = 8.0 Hz, 2H), 6.97 (t, J = 7.3 Hz, 1H), 5.11 (s, 2H), 3.94 (s, 3H). |
| I-236 | 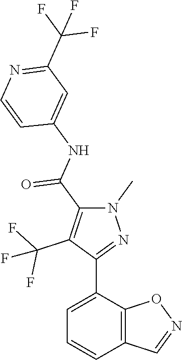 [SMILES: Cn1nc(-c2cccc3cnoc23)c(C(F)(F)F)c1C(=O)Nc1ccnc(C(F)(F)F)c1] | 3-(benzo[d]isoxazol- 7-yl)-N,1-dimethyl-4- (trifluoromethyl)-1H- pyrazole-5- carboxamide | (ES, m/z): [M + H]+ 456.2, ¹H NMR (400 MHz, DMSO) 8.75 (d, J = 5.6 Hz, 1H), 8.19 (d, J = 1.8 Hz, 1H), 7.89 (dd, J = 1.8, 5.3 Hz, 1H), 7.71 (d, J = 6.6 Hz, 1H), 7.46 (d, J = 7.1 Hz, 1H), 7.01 (s, 1H), 3.98 (s, 3H). exchangeable protons and isoxazole very broad |
| I-282 | 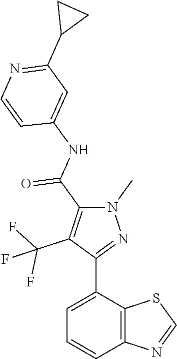 [SMILES: Cn1nc(-c2cccc3ncsc23)c(C(F)(F)F)c1C(=O)Nc1ccnc(C2CC2)c1] | methyl 3- (benzo[d]thiazol-7- yl)-1-methyl-4- (trifluoromethyl)-1H- pyrazole-5- carboxylate | (ES, m/z): [M + H]+ 444.2, ¹H NMR (400 MHz, DMSO) 11.54 (s, 1H), 9.52 (s, 1H), 8.39 (d, J = 5.1 Hz, 1H), 8.25 (dd, J = 1.0, 7.9 Hz, 1H), 7.79- 7.70 (m, 2H), 7.64 (s, 1H), 7.44-7.40 (m, 1H), 4.07 (s, 3H), 2.11 (m, 1H), 0.99-0.95 (m, 4H). |
| I-283 | 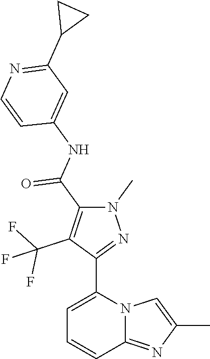 [SMILES: Cc1cn2c(-c3nn(C)c(C(=O)Nc4ccnc(C5CC5)c4)c3C(F)(F)F)cccc2n1] | methyl 1-methyl-3- (2- methylimidazo[1,2- a]pyridin-5-yl)-4- (trifluoromethyl)-1H- pyrazole-5- carboxylate | (ES, m/z): [M + H]+ 441.2, ¹H NMR (400 MHz, DMSO) 11.45 (s, 1H), 8.38 (d, J = 5.6 Hz, 1H), 7.63-7.58 (m, 3H), 7.40 (dd, J = 1.9, 5.5 Hz, 1H), 7.33 (dd, J = 7.0, 8.9 Hz, 1H), 6.97 (d, J = 6.8 Hz, 1H), 4.06 (s, 3H), 2.35 (s, 3H), 2.14- 2.07 (m, 1H), 0.96- 0.91 (m, 4H). |
| I-284 | 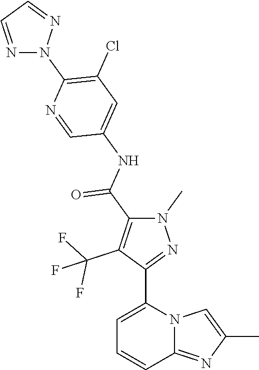 [SMILES: Cc1cn2c(-c3nn(C)c(C(=O)Nc4cnc(-n5nccn5)c(Cl)c4)c3C(F)(F)F)cccc2n1] | methyl 1-methyl-3- (2- methylimidazo[1,2- a]pyridin-5-yl)-4- (trifluoromethyl)-1H- pyrazole-5- carboxylate | (ES, m/z): [M + H]+ 502.2, ¹H NMR (400 MHz, DMSO) 11.94 (s, 1H), 8.78 (s, 1H), 8.65 (d, J = 2.0 Hz, 1H), 8.19 (s, 2H), 7.63- 7.59 (m, 2H), 7.33 (dd, J = 6.9, 9.0 Hz, 1H), 6.98 (d, J = 7.1 Hz, 1H), 4.12 (s, 3H), 2.35 (s, 3H). |
| I-285 | 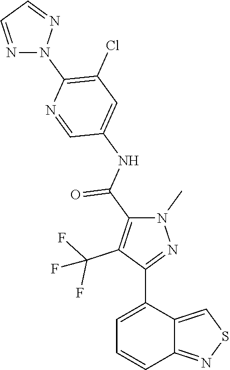 [SMILES: Cn1nc(-c2cccc3nscc23)c(C(F)(F)F)c1C(=O)Nc1cnc(-n2nccn2)c(Cl)c1] | methyl 3- (benzo[c]isothiazol- 4-yl)-1-methyl-4- (trifluoromethyl)-1H- pyrazole-5- carboxylate | (ES, m/z): [M + H]+ 505.2, ¹H NMR (400 MHz, DMSO) 12.00 (s, 1H), 9.81 (d, J = 1.0 Hz, 1H), 8.79 (s, 1H), 8.65 (d, J = 2.3 Hz, 1H), 8.20-8.20 (m, 2H), 7.95-7.92 (m, 1H), 7.65 (dd, J = 6.8, 9.1 Hz, 1H), 7.42 (d, J = 6.8 Hz, 1H), 4.10 (s, 3H). |
| I-286 | 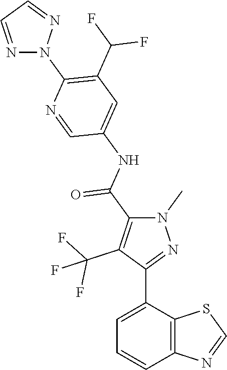 [SMILES: Cn1nc(-c2cccc3ncsc23)c(C(F)(F)F)c1C(=O)Nc1cnc(-n2nccn2)c(C(F)F)c1] | methyl 3- (benzo[d]thiazol-7- yl)-1-methyl-4- (trifluoromethyl)-1H- pyrazole-5- carboxylate | (ES, m/z): [M + H]+ 521.2, ¹H NMR (400 MHz, DMSO) 12.04 (s, 1H), 9.55 (s, 1H), 9.02 (s, 1H), 8.81- 8.79 (m, 1H), 8.30 (s, 2H), 8.27 (dd, J = 1.3, 7.7 Hz, 1H), 7.82- 7.73 (m, 2H), 7.47 (t, J = 54.2 Hz, 1H), 4.16 (s, 3H). |
| I-287 | 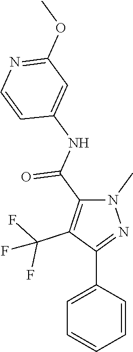 [SMILES: COc1cc(NC(=O)c2c(C(F)(F)F)c(-c3ccccc3)nn2C)ccn1] | ethyl 1-methyl-3- phenyl-4- (trifluoromethyl)-1H- pyrazole-5- carboxylate | (ES, m/z): [M + H]+ 377.2, ¹H NMR (400 MHz, DMSO) 11.52 (s, 1H), 8.20 (d, J = 6.3 Hz, 1H), 7.64 (dd, J = 1.5, 7.5 Hz, 2H), 7.60-7.54 (m, 3H), 7.27-7.23 (m, 2H), 4.01 (s, 3H), 3.92 (s, 3H). |
| I-288 | 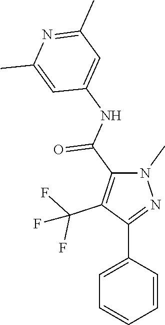 | ethyl 1-methyl-3- phenyl-4- (trifluoromethyl)-1H- pyrazole-5- carboxylate | (ES, m/z): [M + H]+ 375.2, ¹H NMR (400 MHz, DMSO) 11.38 (s, 1H), 7.64 (dd, J = 1.6, 8.1 Hz, 2H), 7.60-7.54 (m, 3H), 7.39-7.38 (m, 2H), 3.99 (s, 3H), 2.47 (s, 6H). |
| I-289 | 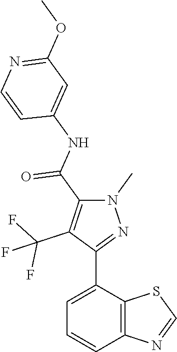 [SMILES: COc1cc(NC(=O)c2c(C(F)(F)F)c(-c3cccc4ncsc34)nn2C)ccn1] | methyl 3- (benzo[d]thiazol-7- yl)-1-methyl-4- (trifluoromethyl)-1H- pyrazole-5- carboxylate | (ES, m/z): [M + H]+ 434.5, ¹H NMR (400 MHz, DMSO) 11.54 (s, 1H), 9.48 (s, 1H), 8.21 (dd, J = 1.4, 7.8 Hz, 1H), 8.15 (d, J = 5.5 Hz, 1H), 7.75- 7.66 (m, 2H), 7.20 (d, J = 5.8 Hz, 2H), 4.03 (s, 3H), 3.87 (s, 3H). |
| I-290 | 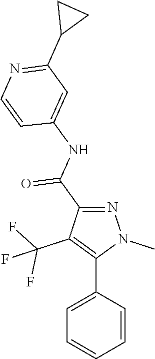 [SMILES: Cn1nc(C(=O)Nc2ccnc(C3CC3)c2)c(C(F)(F)F)c1-c1ccccc1] | ethyl 1-methyl-5- phenyl-4- (trifluoromethyl)-1H- pyrazole-3- carboxylate | (ES, m/z): [M + H]+ 387.4, ¹H NMR (400 MHz, DMSO) 10.75 (1H, s), 8.29 (1H, d, J = 5.6 Hz), 7.75 (1H, d, J = 1.5 Hz), 7.61- 7.57 (3H, m), 7.54- 7.49 (3H, m), 3.75 (3H, s), 2.08-2.00 (1H, m), 0.96-0.88 (4H, m). |

### Example 8—Synthesis of N-(5-chloro-6-(2H-1,2,3-triazol-2-yl) pyridin-3-yl)-3-(isoquinolin-5-yl)-1-methyl-4-(trifluoromethyl)-1H-pyrazole-5-carboxamide (I-140)

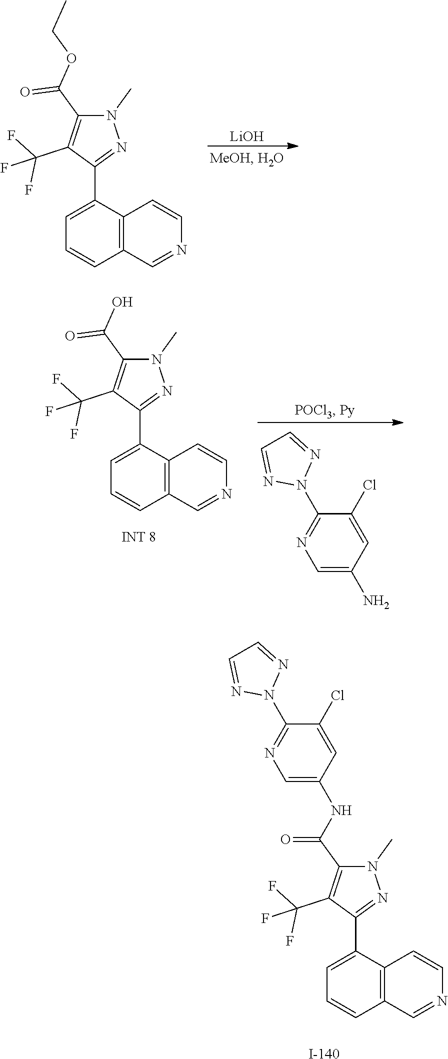

Step 1: Synthesis of 3-(isoquinolin-5-yl)-1-methyl-4-(trifluoromethyl)-1H-pyrazole-5-carboxylic acid INT 8: LiOH (3.6 mg, 0.085 mmol, 1.2 eq) was added to a stirred solution of ethyl 3-(isoquinolin-5-yl)-1-methyl-4-(trifluoromethyl)-1H-pyrazole-5-carboxylate (25 mg, 0.072 mmol, 1.0 eq) in MeOH (5 mL) and water (5 mL). The reaction was stirred at rt for 16 hours. The reaction was evaporated to dryness and the residue was carried forward to the next step without further purification. (ES, m/z): [M+H]⁺ 321.9.

Step 2: Synthesis of N-(5-chloro-6-(2H-1,2,3-triazol-2-yl) pyridin-3-yl)-3-(isoquinolin-5-yl)-1-methyl-4-(trifluoromethyl)-1H-pyrazole-5-carboxamide (I-140): POCl₃ (20 μL, 0.215 mmol, 3.00 eq) was added to a stirred solution of 3-(isoquinolin-5-yl)-1-methyl-4-(trifluoromethyl)-1H-pyrazole-5-carboxylic acid (23 mg, 0.0716 mmol, 1.00 eq) and 5-chloro-6-(2H-1,2,3-triazol-2-yl) pyridin-3-amine (28 mg, 0.143 mmol, 2.00 eq) in pyridine (3 mL), the reaction was then stirred at rt for 1 hour. The reaction was quenched with saturated aqueous NaHCO₃ and EtOAc, the layers were separated, and the organic phase was washed with water and brine. The combined organics were dried (MgSO₄) and concentrated under reduced pressure. The crude residue was purified by reverse phase preparative HPLC to give the title compound (3.9 mg, 0.008 mmol, 11%). (ES, m/z): [M+H]⁺ 499.2 ¹H NMR (400 MHz, DMSO) δ 11.03 (br s, 1H), 9.46 (d, J=0.8 Hz, 1H), 8.79 (br s, 1H), 8.68 (d, J=2.2 Hz, 1H), 8.59 (d, J=6.0 Hz, 1H), 8.35-8.29 (m, 1H), 8.21 (s, 2H), 7.88-7.81 (m, 2H), 7.67 (d, J=6.0 Hz, 1H), 4.12 (s, 3H).

### Example 9—Synthesis of Additional Compounds

Compounds in Table 7 were prepared using a procedure analogous to that described in Example 8.

TABLE 7

| Compound | | Starting | |
|---|---|---|---|
| No. | Structure | Materials | Analytical Data |
| I-141 | 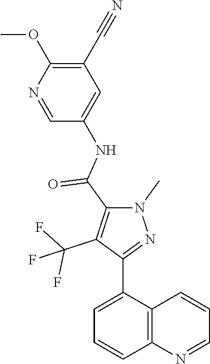 [SMILES: COc1ncc(NC(=O)c2c(C(F)(F)F)c(-c3cccc4ncccc34)nn2C)cc1C#N] | 1-methyl-3-(quinolin-5-yl)-4- (trifluoromethyl)-1H-pyrazole-5- carboxylic acid and 5-amino-2- methoxynicotinonitrile | (ES, m/z): [M + H]⁺ 453.6, ¹H NMR (400 MHz, DMSO) δ 11.50 (s, 1H), 8.98 (dd, J = 1.6, 4.2 Hz, 1H), 8.67 (d, J = 2.5 Hz, 1H), 8.56 (d, J = 2.8 Hz, 1H), 8.17 (d, J = 8.3 Hz, 2H), 7.88 (dd, J = 7.2, 8.5 Hz, 1H), 7.65 (d, J = 7.1 Hz, 1H), 7.60 (dd, J = 4.3, 8.6 Hz, 1H), 4.06 (s, 3H), 4.02 (s, 3H) |
| I-142 | 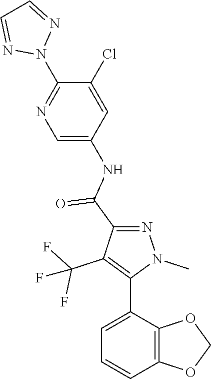 [SMILES: Cn1nc(C(=O)Nc2cnc(-n3nccn3)c(Cl)c2)c(C(F)(F)F)c1-c1cccc2c1OCO2] | 5-(benzo[d][1,3]dioxol-4-yl)-1- methyl-4-(trifluoromethyl)-1H- pyrazole-3-carboxylic acid and 5-chloro-6-(2H-1,2,3-triazol-2- yl)pyridin-3-amine | (ES, m/z): [M + H]⁺ 492.2 ¹H NMR (400 MHz, DMSO) δ 11.28 (s, 1H), 8.92 (br s, 1H), 8.74 (d, J = 2.3 Hz, 1H), 8.19 (s, 2H), 7.16 (dd, J = 7.7, 1.2 Hz, 1H), 7.05 (t, J = 7.8 Hz, 1H), 6.97 (dd, J = 7.9, 1.2 Hz, 1H), 6.14 (s, 2H), 3.84 (s, 3H) |
| I-143 | 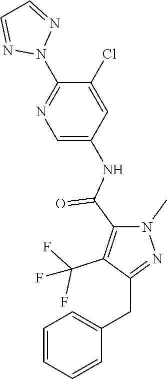 [SMILES: Cn1nc(Cc2ccccc2)c(C(F)(F)F)c1C(=O)Nc1cnc(-n2nccn2)c(Cl)c1] | 3-benzyl-1-methyl-4- (trifluoromethyl)-1H-pyrazole-5- carboxylic acid and 5-chloro-6- (2H-1,2,3-triazol-2-yl)pyridin-3- amine | (ES, m/z): [M + H]⁺ 462.2 ¹H NMR (400 MHz, DMSO) δ 11.79 (br s, 1H), 8.69 (br s, 1H), 8.60 (d, J = 2.0 Hz, 1H), 8.18 (s, 2H), 7.36-7.20 (m, 5H), 4.04 (s, 2H), 3.93 (s, 3H) |
| I-144 | 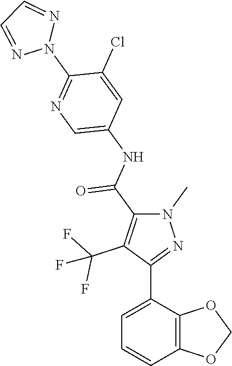 [SMILES: Cn1nc(-c2cccc3c2OCO3)c(C(F)(F)F)c1C(=O)Nc1cnc(-n2nccn2)c(Cl)c1] | 3-(benzo[d][1,3]dioxol-4-yl)-1- methyl-4-(trifluoromethyl)-1H- pyrazole-5-carboxylic acid and 5-chloro-6-(2H-1,2,3-triazol-2- yl)pyridin-3-amine | (ES, m/z): [M + H]⁺ 492.2 ¹H NMR (400 MHz, DMSO) δ 11.96 (br s, 1H), 8.76 (br s, 1H), 8.64 (d, J = 2.2 Hz, 1H), 8.20 (s, 2H), 7.06 (dd, J = 7.6, 1.4 Hz, 1H), 6.97 (t, J = 7.6 Hz, 1H), 6.92 (dd, J = 7.7, 1.4 Hz, 1H), 6.08 (s, 2H), 4.04 (s, 3H) |
| I-145 | 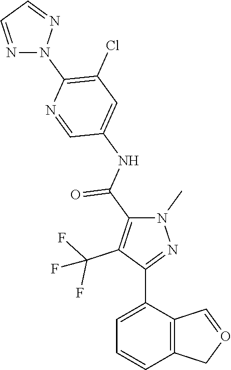 | 3-(benzofuran-4-yl)-1-methyl-4- (trifluoromethyl)-1H-pyrazole-5- carboxylic acid and 5-chloro-6- (2H-1,2,3-triazol-2-yl)pyridin-3- amine | (ES, m/z): [M + H]⁺ 488.2, ¹H NMR (400 MHz, DMSO) δ 11.95 (s, 1H), 8.79 (s, 1H), 8.64 (d, J = 2.3 Hz, 1H), 8.20 (s, 2H), 8.10 (d, J = 2.1 Hz, 1H), 7.74 (d, J = 8.2 Hz, 1H), 7.46 (dd, J = 7.8, 7.8 Hz, 1H), 7.40 (d, J = 7.4 Hz, 1H), 7.06 (d, J = 1.6 Hz, 1H), 4.08 (s, 3H) |
| I-146 | 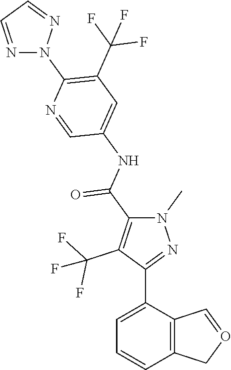 | 3-(benzofuran-4-yl)-1-methyl-4- (trifluoromethyl)-1H-pyrazole-5- carboxylic acid and 6-(2H-1,2,3- triazol-2-yl)-5- (trifluoromethyl)pyridin-3-amine | (ES, m/z): [M + H]⁺ 522.2, ¹H NMR (400 MHz, DMSO) δ 12.09 (s, 1H), 9.09 (s, 1H), 8.86 (d, J = 2.4 Hz, 1H), 8.22 (s, 2H), 8.11 (d, J = 2.3 Hz, 1H), 7.74 (d, J = 8.2 Hz, 1H), 7.47 (dd, J = 7.8, 7.8 Hz, 1H), 7.41 (d, J = 7.4 Hz, 1H), 7.07 (d, J = 1.6 Hz, 1H), 4.09 (s, 3H) |
| I-147 | 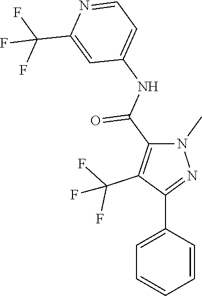 [SMILES: Cn1nc(-c2ccccc2)c(C(F)(F)F)c1C(=O)Nc1ccnc(C(F)(F)F)c1] | 1-methyl-3-phenyl-4- (trifluoromethyl)-1H-pyrazole-5- carboxylic acid and 2- (trifluoromethyl)pyridin-4-amine | (ES, m/z): [M + H]⁺ 415.2, ¹H NMR (400 MHz, DMSO) δ 11.97 (s, 1H), 8.75 (d, J = 5.5 Hz, 1H), 8.19 (d, J = 1.5 Hz, 1H), 7.88 (dd, J = 0.4, 3.7 Hz, 1H), 7.61 (dd, J = 1.7, 7.8 Hz, 2H), 7.56-7.49 (m, 3H), 4.00 (s, 3H) |
| I-148 | 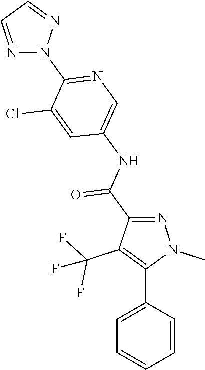 [SMILES: Cn1nc(C(=O)Nc2cnc(-n3nccn3)c(Cl)c2)c(C(F)(F)F)c1-c1ccccc1] | 1-methyl-5-phenyl-4- (trifluoromethyl)-1H-pyrazole-3- carboxylic acid and 5-chloro-6- (2H-1,2,3-triazol-2-yl)pyridin-3- amine | (ES, m/z): [M + H]⁺ 448.2, ¹H NMR (400 MHz, DMSO) δ 11.30 (s, 1H), 9.01 (d, J = 2.3 Hz, 1H), 8.77-8.75 (m, 1H), 8.20- 8.20 (m, 2H), 7.63-7.59 (m, 3H), 7.57-7.53 (m, 2H), 3.80 (s, 3H) |
| I-149 | 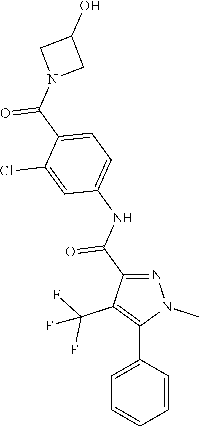 [SMILES: Cn1nc(C(=O)Nc2ccc(C(=O)N3CC(O)C3)c(Cl)c2)c(C(F)(F)F)c1-c1ccccc1] | 1-methyl-5-phenyl-4- (trifluoromethyl)-1H-pyrazole-3- carboxylic acid and (4-amino-2- chlorophenyl)(3- hydroxyazetidin-1-yl)methanone | (ES, m/z): [M + H]⁺ 479.2, ¹H NMR (400 MHz, DMSO) δ 10.87 (s, 1H), 8.06 (d, J = 1.9 Hz, 1H), 7.81 (dd, J = 2.0, 8.4 Hz, 1H), 7.63-7.58 (m, 3H), 7.55-7.51 (m, 2H), 7.43- 7.40 (m, 1H), 5.83 (d, J = 5.6 Hz, 1H), 4.55-4.50 (m, 1H), 4.24 (dd, J = 6.9, 10.2 Hz, 1H), 4.06 (t, J = 7.8 Hz, 1H), 3.79 (d, J = 5.0 Hz, 1H), 3.77 (s, 3H), 3.72 (dd, J = 4.5, 8.9 Hz, 1H) |
| I-150 | 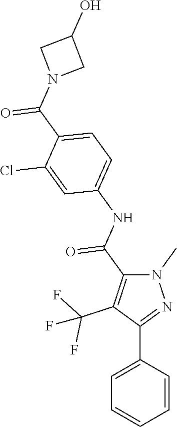 [SMILES: Cn1nc(-c2ccccc2)c(C(F)(F)F)c1C(=O)Nc1ccc(C(=O)N2CC(O)C2)c(Cl)c1] | 1-methyl-3-phenyl-4- (trifluoromethyl)-1H-pyrazole-5- carboxylic acid and (4-amino-2- chlorophenyl)(3- hydroxyazetidin-1-yl)methanone | (ES, m/z): [M + H]⁺ 479.2, ¹H NMR (400 MHz, DMSO) δ 11.45 (s, 1H), 7.95-7.93 (m, 1H), 7.66- 7.60 (m, 2H), 7.56-7.45 (m, 5H), 5.83 (d, J = 6.1 Hz, 1H), 4.57-4.49 (m, 1H), 4.25 (dd, J = 7.0, 10.2 Hz, 1H), 4.07 (t, J = 8.0 Hz, 1H), 3.99 (s, 3H), 3.78 (dd, J = 4.5, 10.5 Hz, 1H), 3.72 (dd, J = 4.4, 9.1 Hz, 1H). |
| I-151 | 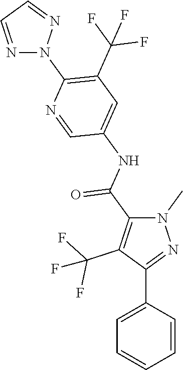 [SMILES: Cn1nc(-c2ccccc2)c(C(F)(F)F)c1C(=O)Nc1cnc(-n2nccn2)c(C(F)(F)F)c1] | 1-methyl-3-phenyl-4- (trifluoromethyl)-1H-pyrazole-5- carboxylic acid and 6-(2H-1,2,3- triazol-2-yl)-5- (trifluoromethyl)pyridin-3- amine | (ES, m/z): [M + H]⁺ 482, ¹H NMR (400 MHz, DMSO) δ 12.05 (s, 1H), 9.07 (s, 1H), 8.84 (d, J = 2.3 Hz, 1H), 8.22-8.21 (m, 2H), 7.61 (dd, J = 1.4, 7.7 Hz, 2H), 7.55-7.51 (m, 3H), 4.03 (s, 3H) |
| I-152 | 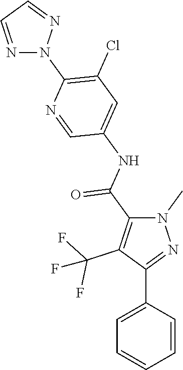 [SMILES: Cn1nc(-c2ccccc2)c(C(F)(F)F)c1C(=O)Nc1cnc(-n2nccn2)c(Cl)c1] | 1-methyl-3-phenyl-4- (trifluoromethyl)-1H-pyrazole-5- carboxylic acid and 5-chloro-6- (2H-1,2,3-triazol-2-yl)pyridin-3- amine | (ES, m/z): [M + H]⁺ 448.2, ¹H NMR (400 MHz, DMSO) δ 11.95-11.92 (m, 1H), 8.79 (s, 1H), 8.64 (d, J = 2.3 Hz, 1H), 8.22- 8.21 (m, 2H), 7.62 (dd, J = 1.6, 7.6 Hz, 2H), 7.57- 7.50 (m, 3H), 4.04 (s, 3H) |
| I-153 | 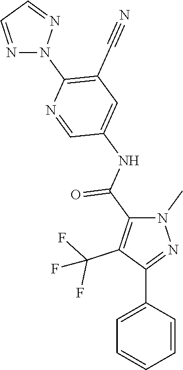 [SMILES: Cn1nc(-c2ccccc2)c(C(F)(F)F)c1C(=O)Nc1cnc(-n2nccn2)c(C#N)c1] | 1-methyl-3-phenyl-4- (trifluoromethyl)-1H-pyrazole-5- carboxylic acid and 5-amino-2- (2H-1,2,3-triazol-2- yl)nicotinonitrile | (ES, m/z): [M + H]⁺ 439.2, ¹H NMR (400 MHz, DMSO) δ 12.03 (s, 1H), 8.99 (s, 1H), 8.87-8.82 (m, 1H), 8.32 (s, 2H), 7.63- 7.49 (m, 5H), 4.02 (s, 3H) |
| I-154 | 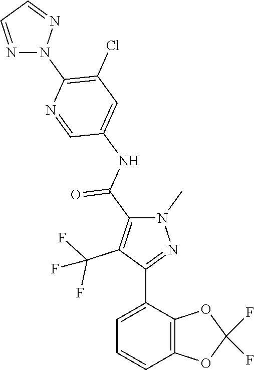 [SMILES: Cn1nc(-c2cccc3c2OC(F)(F)O3)c(C(F)(F)F)c1C(=O)Nc1cnc(-n2nccn2)c(Cl)c1] | 3-(2,2- difluorobenzo[d][1,3]dioxol-4- yl)-1-methyl-4-(trifluoromethyl)- 1H-pyrazole-5-carboxylic acid and 5-chloro-6-(2H-1,2,3-triazol- 2-yl)pyridin-3-amine | (ES, m/z): [M + H]⁺ 528.2, ¹H NMR (400 MHz, DMSO) δ 11.95-11.91 (m, 1H), 8.77 (s, 1H), 8.62 (d, J = 2.3 Hz, 1H), 8.20- 8.19 (m, 2H), 7.57 (dd, J = 1.3, 7.8 Hz, 1H), 7.40- 7.32 (m, 2H), 4.07 (s, 3H) |
| I-155 | 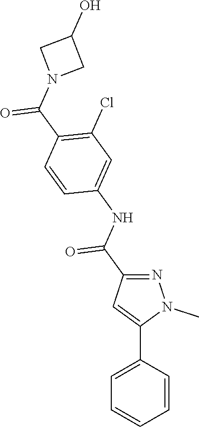 [SMILES: Cn1nc(C(=O)Nc2ccc(C(=O)N3CC(O)C3)c(Cl)c2)cc1-c1ccccc1] | 1-methyl-5-phenyl-1H-pyrazole- 3-carboxylic acid and (4-amino- 2-chlorophenyl)(3- hydroxyazetidin-1-yl)methanone | (ES, m/z): [M + H]⁺ 411.2, ¹H NMR (400 MHz, DMSO) δ 10.49 (s, 1H), 8.11 (d, J = 1.9 Hz, 1H), 7.88 (dd, J = 2.0, 8.5 Hz, 1H), 7.66-7.63 (m, 2H), 7.59-7.51 (m, 3H), 7.39 (d, J = 8.4 Hz, 1H), 6.97 (s, 1H), 5.82 (d, J = 5.3 Hz, 1H), 4.53-4.48 (m, 1H), 4.24 (dd, J = 7.1, 9.9 Hz, 1H), 4.10-4.04 (m, 1H), 4.00 (s, 3H), 3.79-3.69 (s, 2H) |
| I-156 | 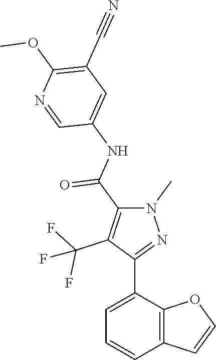 [SMILES: COc1ncc(NC(=O)c2c(C(F)(F)F)c(-c3cccc4ccoc34)nn2C)cc1C#N] | 3-(benzofuran-7-yl)-1-methyl-4- (trifluoromethyl)-1H-pyrazole-5- carboxylic acid and 5-amino-2- methoxynicotinonitrile | (ES, m/z): [M + H]⁺ 442.2, ¹H NMR (400 MHz, DMSO) δ 11.53 (s, 1H), 8.68 (d, J = 2.6 Hz, 1H), 8.53 (d, J = 2.6 Hz, 1H), 8.04 (d, J = 2.2 Hz, 1H), 7.82-7.76 (m, 1H), 7.39- 7.34 (m, 2H), 7.06 (d, J = 2.2 Hz, 1H), 4.03 (s, 3H), 4.02 (s, 3H) |
| I-157 | 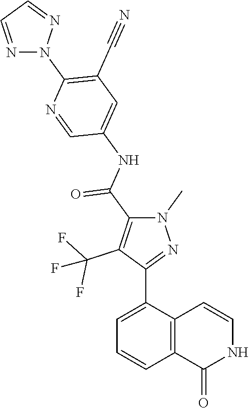 [SMILES: Cn1nc(-c2cccc3c(=O)[nH]ccc23)c(C(F)(F)F)c1C(=O)Nc1cnc(-n2nccn2)c(C#N)c1] | 1-methyl-3-(1-oxo-1,2- dihydroisoquinolin-5-yl)-4- (trifluoromethyl)-1H-pyrazole-5- carboxylic acid and 5-amino-2- (2H-1,2,3-triazol-2- yl)nicotinonitrile | (ES, m/z): [M + H]⁺ 506.2, ¹H NMR (400 MHz, DMSO) δ 11.96 (s, 1H), 11.43 (d, J = 5.1 Hz, 1H), 9.05-8.99 (m, 1H), 8.85 (d, J = 2.5 Hz, 1H), 8.36- 8.30 (m, 3H), 7.69 (d, J = 6.8 Hz, 1H), 7.59 (t, J = 7.7 Hz, 1H), 7.22 (t, J = 6.4 Hz, 1H), 6.29 (d, J = 7.1 Hz, 1H), 4.07 (s, 3H) |
| I-207 | 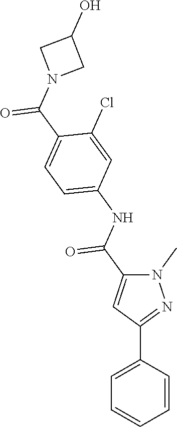 [SMILES: Cn1nc(-c2ccccc2)cc1C(=O)Nc1ccc(C(=O)N2CC(O)C2)c(Cl)c1] | 1-methyl-3-phenyl-1H-pyrazole- 5-carboxylic acid and (4-amino- 2-chlorophenyl)(3- hydroxyazetidin-1-yl)methanone | (ES, m/z): [M + H]+ 411.2, H NMR (400 MHz, DMSO) 10.59 (1H, s), 8.01 (1H, d, J = 2.0 Hz), 7.83 (2H, d, J = 7.3 Hz), 7.76 (1H, dd, J = 2.0, 8.4 Hz), 7.51-7.44 (4H, m), 7.37 (1H, dd, J = 7.4, 7.4 Hz), 5.83 (1H, d, J = 6.1 Hz), 4.56-4.49 (1H, m), 4.25 (1H, dd, J = 7.3, 10.0 Hz), 4.16 (3H, s), 4.06 (1H, dd, J = 7.9, 7.9 Hz), 3.75 (2H, ddt, J = 4.6, 11.2, 11.9 Hz) |
| I-86 | 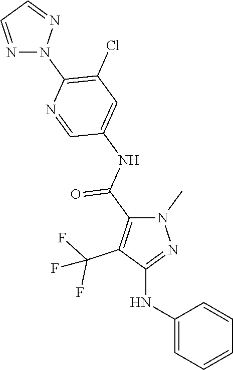 [SMILES: Cn1nc(Nc2ccccc2)c(C(F)(F)F)c1C(=O)Nc1cnc(-n2nccn2)c(Cl)c1] | ethyl 1-methyl-3- (phenylamino)-4- (trifluoromethyl)-1H- pyrazole-5-carboxylate | (ES, m/z): [M + H]+ 463.2, ¹H NMR (400 MHz, DMSO) 8.60 (d, J = 2.0 Hz, 1H), 8.16 (s, 2H), 7.40-7.37 (m, 2H), 7.21 (dd, J = 7.8, 7.8 Hz, 3H), 6.81 (s, 1H), 3.86 (s, 3H) 2 NH exchangeable protons not observed |
| I-235 | 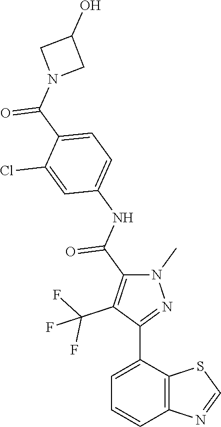 [SMILES: Cn1nc(-c2cccc3ncsc23)c(C(F)(F)F)c1C(=O)Nc1ccc(C(=O)N2CC(O)C2)c(Cl)c1] | 3-(benzo[d]thiazol-7-yl)-1- methyl-4-(trifluoromethyl)- 1H-pyrazole-5-carboxylic acid | (ES, m/z): [M + H]+ 536.2, ¹H NMR (400 MHz, DMSO) 11.53 (s, 1H), 9.48 (s, 1H), 8.21 (dd, J = 1.1, 8.0 Hz, 1H), 7.93 (d, J = 1.8 Hz, 1H), 7.75-7.61 (m, 3H), 7.50-7.45 (m, 1H), 5.82 (d, J = 6.3 Hz, 1H), 4.54-4.49 (m, 1H), 4.24 (dd, J = 7.2, 10.5 Hz, 1H), 4.09-4.05 (m, 1H), 4.04 (s, 3H), 3.77 (dd, J = 4.5, 10.2 Hz, 1H), 3.71 (dd, J = 4.3, 9.2 Hz, 1H). |
| I-291 | 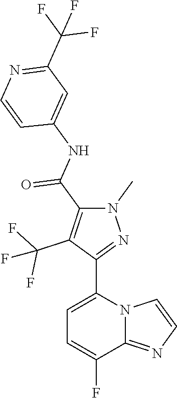 [SMILES: Cn1nc(-c2ccc(F)c3nccn23)c(C(F)(F)F)c1C(=O)Nc1ccnc(C(F)(F)F)c1] | 3-(8-fluoroimidazo[1,2- a]pyridin-5-yl)-1-methyl-4- (trifluoromethyl)-1H- pyrazole-5-carboxylic acid | (ES, m/z): [M + H]+ 473.6, ¹H NMR (400 MHz, DMSO) 11.94 (s, 1H), 8.77 (d, J = 5.6 Hz, 1H), 8.19 (d, J = 1.8 Hz, 1H), 8.02 (dd, J = 1.0, 3.3 Hz, 1H), 7.90-7.87 (m, 1H), 7.74 (d, J = 1.3 Hz, 1H), 7.33 (dd, J = 7.8, 10.9 Hz, 1H), 7.08- 7.03 (m, 1H), 4.10 (s, 3H). |

### Example 10—Synthesis of 3-(2,3-dihydrobenzofuran-7-yl)-1-methyl-4-(trifluoromethyl)-N-(2-(trifluoromethyl)pyridin-4-yl)-1H-pyrazole-5-carboxamide (I-158)

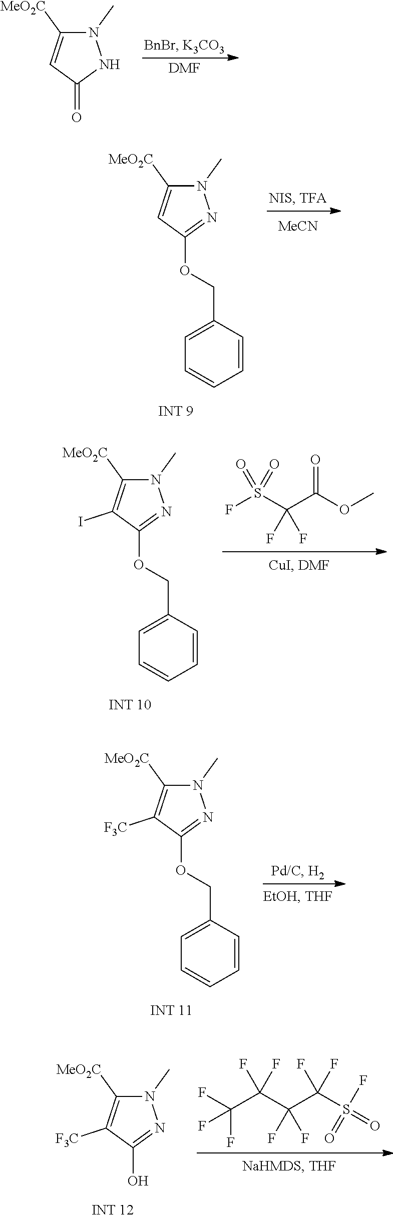

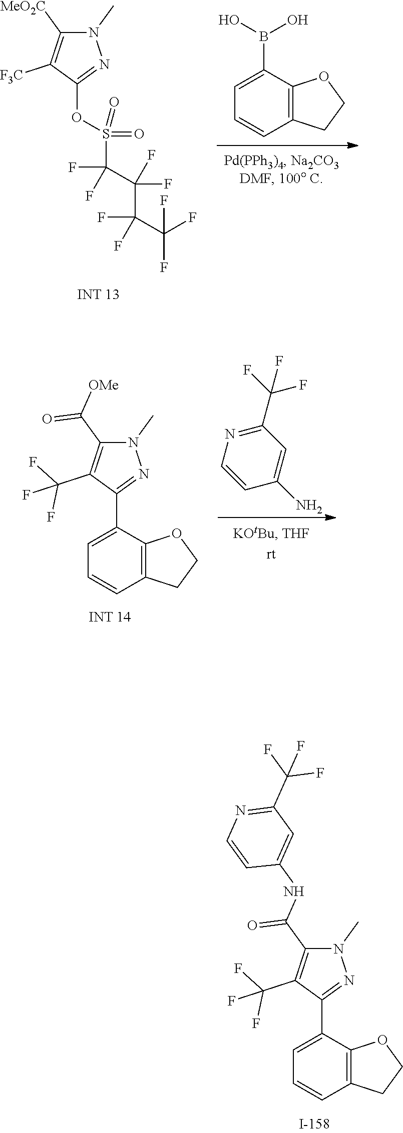 [SMILES: COC(=O)c1c(C(F)(F)F)c(OS(=O)(=O)C(F)(F)C(F)(F)C(F)(F)C(F)(F)F)nn1C ; OB(O)c1cccc2c1OCC2 ; COC(=O)c1c(C(F)(F)F)c(-c2cccc3c2OCC3)nn1C ; Nc1ccnc(C(F)(F)F)c1 ; Cn1nc(-c2cccc3c2OCC3)c(C(F)(F)F)c1C(=O)Nc1ccnc(C(F)(F)F)c1]

Step 1: Synthesis of methyl 3-(benzyloxy)-1-methyl-1H-pyrazole-5-carboxylate (INT 9): Benzyl bromide (2.4 mL, 20.2 mmol, 1.05 eq) was added dropwise to a stirred mixture of methyl 2-methyl-5-oxo-1H-pyrazole-3-carboxylate (3.00 g, 19.2 mmol, 1.00 eq) and K₂CO₃ (3.19 g, 23.1 mmol, 1.20 eq) in DMF (20 mL) and the reaction was stirred at rt for 16 hours. The mixture was poured into 1 M aqueous HCl and extracted with EtOAc. The organic phase was separated, washed with brine, dried (MgSO₄) and concentrated. The crude residue was purified by column chromatography eluting with 0-50% EtOAc in cyclohexane to give the title compound (3.8 g, 15.4 mmol, 80%). (ES, m/z): [M+H]⁺ 247.3, ¹H NMR (400 MHz, CDCl₃) δ 7.44 (d, J=7.2 Hz, 2H), 7.39-7.31 (m, 3H), 6.21 (s, 1H), 5.18 (s, 2H), 4.05 (s, 3H), 3.85 (s, 3H).

Step 2: Synthesis of methyl 3-(benzyloxy)-4-iodo-1-methyl-1H-pyrazole-5-carboxylate (INT 10): TFA (1.7 mL, 21.6 mmol, 1.40 eq) was added to a stirred mixture of methyl 3-(benzyloxy)-1-methyl-1H-pyrazole-5-carboxylate (3.80 g, 15.4 mmol, 1.00 eq) and NIS (4.17 g, 18.5 mmol, 1.20 eq) in MeCN (60 mL), the reaction was then stirred at 60° C. for 2 hours. The reaction was cooled to rt and concentrated. The crude residue was dissolved in EtOAc and the solution washed with 1 M aqueous Na₂S203, saturated aqueous NaHCO₃ and brine. The organic phase was dried and concentrated to give the title compound as a white solid (5.4 g, 14.5 mmol, 94%). No mass ion, ¹H NMR (400 MHz, CDCl₃) δ 7.47 (d, J=7.3 Hz, 2H), 7.40-7.31 (m, 3H), 5.28 (s, 2H), 4.07 (s, 3H), 3.92 (s, 3H).

Step 3: Synthesis of methyl 3-(benzyloxy)-1-methyl-4-(trifluoromethyl)-1H-pyrazole-5-carboxylate (INT 11): CuI (2.05 g, 10.7 mmol, 2.00 eq) was added to a stirred solution of methyl 3-(benzyloxy)-4-iodo-1-methyl-1H-pyrazole-5-carboxylate (2.00 g, 5.37 mmol, 1.00 eq) and methyl 2,2-difluoro-2-(fluorosulfonyl)acetate (1.4 mL, 10.7 mmol, 2.00 eq) in DMF (30 mL). The reaction mixture was heated to 90° C. and stirred for 10 hours, then cooled to rt, diluted with EtOAc and filtered through Celite®. The filtrate was then washed with saturated aqueous NH₄Cl, saturated aqueous NaHCO₃ and brine. The organic phase was dried and concentrated, the crude residue was purified by column chromatography eluting with 0-50% EtOAc in cyclohexane to give the title compound as a white crystalline solid (1.45 g, 4.6 mmol, 86%). (ES, m/z): [M+H]⁺ 315.3, ¹H NMR (400 MHz, CDCl₃) δ 7.43 (d, J=7.4 Hz, 2H), 7.39-7.31 (m, 3H), 5.30 (s, 211), 4.00 (s, 3H), 3.93 (s, 3H).

Step 4: Synthesis of methyl 3-hydroxy-1-methyl-4-(trifluoromethyl)-1H-pyrazole-5-carboxylate (INT 12): Palladium on carbon (10% w/w, 0.47 g. 0.445 mmol, 0.100 eq) was added to a stirred and degassed solution of methyl 3-(benzyloxy)-1-methyl-4-(trifluoromethyl)-1H-pyrazole-5-carboxylate (1.40 g, 4.45 mmol, 1.0 eq) in EtOH (20 mL) and THF (20 mL). The reaction flask was evacuated and refilled with N₂ three times, and then placed under an H2 environment. The reaction was stirred under an atmosphere of H₂ for 16 hours. The reaction flask was evacuated and refilled with N₂ three times, and then filtered through Celite® under a cone of N₂. The filtrate was concentrated to give the title compound as a white solid (0.92 g, 4.1 mmol, 92%). (ES, m/z): [M+H]⁺ 225.2, ¹H NMR (400 MHz, CDCl₃) δ 10.10 (br s, 1H), 4.01 (s, 3H), 3.96 (s, 3H).

Step 5: Synthesis of methyl 1-methyl-3-(((perfluorobutyl) sulfonyl)oxy)-4-(trifluoromethyl)-1H-pyrazole-5-carboxylate (INT 13): 1 M NaHMDS in THF (2.7 mL, 2.68 mmol, 1.20 eq) was added dropwise to a stirred solution of methyl 3-hydroxy-1-methyl-4-(trifluoromethyl)-1H-pyrazole-5-carboxylate (0.50 g, 2.23 mmol, 1.00 eq) in THF (8 mL) at 0° C. under the N₂. The reaction mixture was stirred at 0° C. for 15 minutes followed by the dropwise addition of perfluoro-1-butanesulfonyl fluoride (0.48 mL, 2.68 mmol, 1.20 eq). The reaction mixture was stirred for 1 hour at 0° C. and then allowed to warm to rt and stirred for 12 hours. The mixture was poured into 1 M aqueous HCl and extracted with EtOAc, the organics were combined, washed with brine, dried and concentrated. The crude residue was purified by column chromatography eluting with 0-40% EtOAc in cyclohexane to give the title compound as a colourless oil (0.78 g, 1.54 mmol, 69%). No mass ion, ¹H NMR (400 MHz, CDCl₃) δ 4.17 (s, 3H), 3.99 (s, 3H).

Step 6: Synthesis of methyl 3-(2,3-dihydrobenzofuran-7-yl)-1-methyl-4-(trifluoromethyl)-1H-pyrazole-5-carboxylate (INT 14): Pd(PPh₃)₄ (34 mg, 0.0296 mmol, 0.10 eq) was added to a stirred degassed mixture of methyl 1-methyl-3-(((perfluorobutyl) sulfonyl)oxy)-4-(trifluoromethyl)-1H-pyrazole-5-carboxylate (150 mg, 0.296 mmol, 1.00 eq), (2,3-dihydrobenzofuran-7-yl) boronic acid (73 mg, 0.444 mmol, 1.50 eq) and Na₂CO₃ (94 mg, 0.889 mmol, 3.00 eq) in DMF (4 mL). The reaction mixture was stirred at 100° C. for 3 hours. The mixture was cooled to rt and diluted with EtOAc and washed with saturated aqueous NaHCO₃ and brine. The organics were dried and concentrated, the crude residue was purified by column chromatography eluting with 0-100% EtOAc in cyclohexane to give the title compound as an off-white solid (100 mg, 0.30 mmol, 100% at 90% purity). (ES, m/z): [M+H]⁺ 327.3, ¹H NMR (400 MHz, CDCl₃) δ 7.25-7.23 (m, 1H), 7.15 (d, J=7.5 Hz, 1H), 6.90 (q, J=7.6 Hz, 1H), 4.59 (t, J=8.8 Hz, 2H), 4.18 (s, 3H), 3.97 (s, 3H), 3.26 (t, J=8.8 Hz, 2H).

Step 7: Synthesis of 3-(2,3-dihydrobenzofuran-7-yl)-1-methyl-4-(trifluoromethyl)-N-(2-(trifluoromethyl)pyridin-4-yl)-1H-pyrazole-5-carboxamide (I-158): The title compound was synthesized from INT 14 based on procedures described in Example 6 and isolated by reverse phase HPLC as an off-white solid (16.5 mg, 0.036 mmol, 23%). (ES, m/z): [M+H]⁺ 457.2, ¹H NMR (400 MHz, DMSO) δ 11.86 (s, 1H), 8.73 (d, J=3.3 Hz, 1H), 8.18 (s, 1H), 7.87-7.87 (m, 1H), 7.33 (dd, J=1.0, 7.3 Hz, 1H), 7.10 (d, J=7.6 Hz, 1H), 6.91 (dd, J=7.5, 7.5 Hz, 1H), 4.52 (dd, J=8.7, 8.7 Hz, 2H), 3.97 (s, 3H), 3.25 (dd, J=8.7, 8.7 Hz, 2H).

### Example 11—Synthesis of Additional Compounds

Compounds in Table 8 were prepared using procedures analogous to those described in Example 10.

TABLE 8

| Compound | | Starting | |
|---|---|---|---|
| No. | Structure | Materials | Analytical Data |
| I-159 | 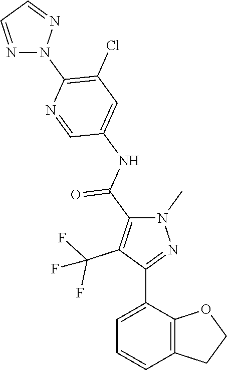 [SMILES: Cn1nc(-c2cccc3c2OCC3)c(C(F)(F)F)c1C(=O)Nc1cnc(-n2nccn2)c(Cl)c1] | methyl 3-(2,3- dihydrobenzofu- ran-7-yl)-1- methyl-4- (trifluoromethyl)- 1H-pyrazole- 5-carboxylate and 5-chloro-6- (2H-1,2,3- triazol-2- yl)pyridin-3- amine | (ES, m/z): [M + H]⁺ 490.2 ¹H NMR (400 MHz, DMSO) δ 11.87 (s, 1H), 8.80-8.78 (m, 1H), 8.62 (d, J = 2.3 Hz, 1H), 8.19 (s, 2H), 7.33 (d, J = 7.6 Hz, 1H), 7.11 (d, J = 7.6 Hz, 1H), 6.92 (dd, J = 7.5, 7.5 Hz, 1H), 4.53 (dd, J = 8.7, 8.7 Hz, 2H), 4.00 (s, 3H), 3.29-3.22 (m, 2H) |
| I-160 | 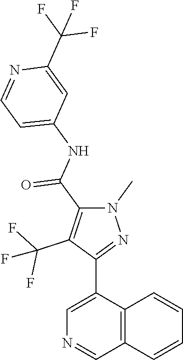 [SMILES: Cn1nc(-c2cncc3ccccc23)c(C(F)(F)F)c1C(=O)Nc1ccnc(C(F)(F)F)c1] | methyl 3- (isoquinolin-4- yl)-1-methyl-4- (trifluoromethyl)- 1H-pyrazole- 5-carboxylate and 2- (trifluoromethyl) pyridin-4- amine | (ES, m/z): [M + H]⁺ 466.3 ¹H NMR (400 MHz, DMSO) δ d 11.98 (s, 1H), 9.47 (s, 1H), 8.78-8.74 (m, 1H), 8.52-8.51 (m, 1H), 8.29- 8.26 (m, 1H), 8.21 (s, 1H), 7.91- 7.77 (m, 4H), 4.09 (s, 3H) |
| I-161 | 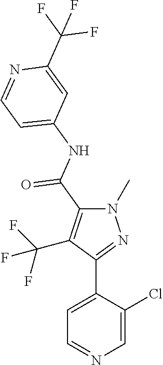 [SMILES: Cn1nc(-c2ccncc2Cl)c(C(F)(F)F)c1C(=O)Nc1ccnc(C(F)(F)F)c1] | methyl 3-(3- chloropyridin- 4-yl)-1-methyl- 4- (trifluoromethyl)- 1H-pyrazole- 5-carboxylate and 2- (trifluoromethyl) pyridin-4- amine | (ES, m/z): [M + H]⁺ 450.3 ¹H NMR (400 MHz, DMSO) δ 11.96 (s, 1H), 8.86 (s, 1H), 8.76 (d, J = 5.5 Hz, 1H), 8.69 (d, J = 4.9 Hz, 1H), 8.19 (d, J = 1.6 Hz, 1H), 7.89 (dd, J = 0.5, 3.9 Hz, 1H), 7.52 (d, J = 4.9 Hz, 1H), 4.06 (s, 3H) |

### Example 12—Synthesis of 3-([1,2,4]triazolo[1,5-a]pyridin-5-yl)-1-methyl-4-(trifluoromethyl)-N-(2-(trifluoromethyl)pyridin-4-yl)-1H-pyrazole-5-carboxamide (I-162)

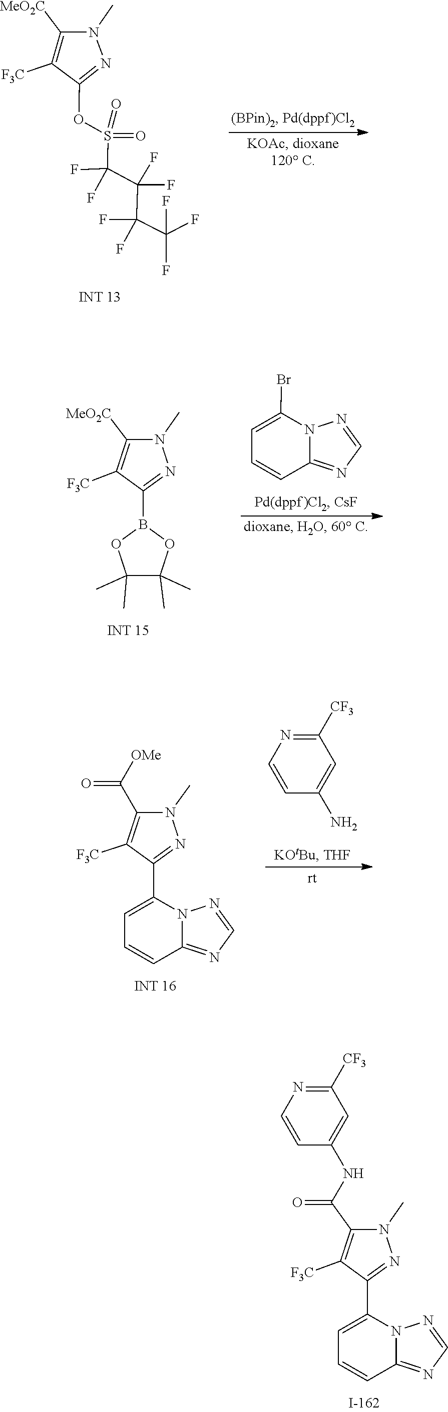 [SMILES: COC(=O)c1c(C(F)(F)F)c(OS(=O)(=O)C(F)(F)C(F)(F)C(F)(F)C(F)(F)F)nn1C ; COC(=O)c1c(C(F)(F)F)c(B2OC(C)(C)C(C)(C)O2)nn1C ; Brc1cccc2ncnn12 ; COC(=O)c1c(C(F)(F)F)c(-c2cccc3ncnn23)nn1C ; Nc1ccnc(C(F)(F)F)c1 ; Cn1nc(-c2cccc3ncnn23)c(C(F)(F)F)c1C(=O)Nc1ccnc(C(F)(F)F)c1]

Step 1: Synthesis of methyl 1-methyl-3-(4,4,5,5-tetramethyl-1,3,2-dioxaborolan-2-yl)-4-(trifluoromethyl)-1H-pyrazole-5-carboxylate (INT 15): Pd(dppf)Cl₂ (147 mg, 0.198 mmol, 0.100 eq) was added to a stirred degassed mixture of methyl 1-methyl-3-(((perfluorobutyl) sulfonyl)oxy)-4-(trifluoromethyl)-1H-pyrazole-5-carboxylate (1,000 mg, 1.98 mmol, 1.00 eq), potassium acetate (582 mg, 5.93 mmol, 3.00 eq) and bis(pinacolato)diboron (752 mg, 2.96 mmol, 1.50 eq) in 1,4-dioxane (12 mL). The reaction mixture was then stirred at 120° C. for 24 hours. The reaction mixture was cooled to rt and diluted with EtOAc (30 mL). The reaction mixture was filtered through Celite® and washed with EtOAc (3×10 mL). The solvent was removed under reduced pressure and the residue was purified by column chromatography with 0-40% EtOAc in cyclohexane to give the title compound as a light yellow oil (476 mg, 1.4 mmol, 72%). ¹H NMR (400 MHz, CDCl₃) δ 4.17 (s, 3H), 3.95 (s, 3H), 1.37 (s, 12H).

Step 2: Synthesis of methyl 3-([1,2,4]triazolo[1,5-a]pyrid-5-yl)-1-methyl-4-(trifluoromethyl)-1H-pyrazole-5-carboxylate (INT 16): Pd(dppf)Cl₂ (22 mg, 0.0299 mmol, 0.100 eq) was added to a degassed solution of methyl 1-methyl-3-(4,4,5,5-tetramethyl-1,3,2-dioxaborolan-2-yl)-4-(trifluoromethyl)-1H-pyrazole-5-carboxylate (100 mg, 0.299 mmol, 1.00 eq), 5-bromo-[1,2,4]triazolo[1,5-a]pyridine (53 mg, 0.269 mmol, 0.900 eq) and CsF (136 mg, 0.898 mmol, 3.00 eq) in 1,4-dioxane (2.0 mL) and water (0.20 mL) the reaction mixture was stirred at 60° C. for 2 h. The reaction was cooled to rt, diluted with EtOAc, and filtered through a pad of Celite®, which was washed with EtOAc. The solvent was removed under reduced pressure and the crude residue was purified by column chromatography eluting with 0-100% EtOAc in cyclohexane to give the title compound as an off-white solid (85 mg, 0.26 mmol, 87%). ¹H NMR (400 MHz, CDCl₃) δ 8.35 (s, 1H), 7.87 (d, J=8.4 Hz, 1H), 7.61 (dd, J=7.1, 9.0 Hz, 1H), 7.15 (d, J=6.7 Hz, 1H), 4.28 (s, 3H), 4.02 (s, 3H).

Step 3: Synthesis of 3-([1,2,4]triazolo[1,5-a]pyridin-5-yl)-1-methyl-4-(trifluoromethyl)-N-(2-(trifluoromethyl)pyridin-4-yl)-1H-pyrazole-5-carboxamide (I-162): The title compound was synthesized from INT 16 based on procedures described in Example 6 and isolated by reverse phase HPLC as an off-white solid (20 mg, 0.044 mmol, 16%). (ES, m/z): [M+H]⁺ 456.2 ¹H NMR (400 MHz, DMSO) δ 12.02 (s, 1H), 8.76 (d, J=5.1 Hz, 1H), 8.54 (s, 1H), 8.20 (d, J=1.3 Hz, 1H), 8.04 (dd, J=1.1, 9.0 Hz, 1H), 7.91 (d, J=4.8 Hz, 1H), 7.81 (dd, J=7.1, 8.8 Hz, 1H), 7.38 (dd, J=1.0, 7.1 Hz, 1H), 4.10 (s, 3H).

### Example 13—Synthesis of Additional Compounds

The compound in Table 9 was prepared based on procedures described in Example 12.

TABLE 9

| Compound | | Starting | |
|---|---|---|---|
| No. | Structure | Materials | Analytical Data |
| I-163 | 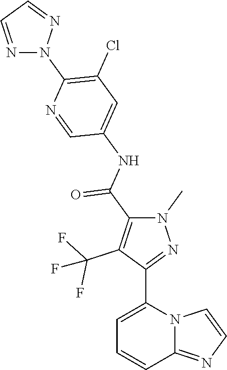 [SMILES: Cn1nc(-c2cccc3nccn23)c(C(F)(F)F)c1C(=O)Nc1cnc(-n2nccn2)c(Cl)c1] | methyl 3- (imidazo[1,2- a]pyridin-5-yl)-1- methyl-4- (trifluoromethyl)- 1H-pyrazole-5- carboxylate and 5-chloro-6-(2H- 1,2,3-triazol-2- yl)pyridin-3- amine | (ES m/z): [M + H]⁺ 488.2, ¹H NMR (400 MHz, DMSO) δ 12.12 (s, 1H), 8.79 (d, J = 2.1 Hz, 1H), 8.66 (d, J = 2.3 Hz, 1H), 8.21 (s, 2H), 7.92 (s, 1H), 7.76 (d, J = 9.0 Hz, 1H), 7.69 (d, J = 1.0 Hz, 1H), 7.41 (dd, J = 7.0, 9.1 Hz, 1H), 7.08 (d, J = 6.8 Hz, 1H), 4.13 (s, 3H) |
| I-222 | 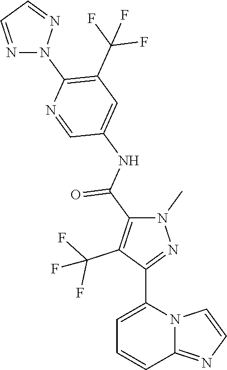 [SMILES: Cn1nc(-c2cccc3nccn23)c(C(F)(F)F)c1C(=O)Nc1cnc(-n2nccn2)c(C(F)(F)F)c1] | methyl 3- (imidazo[1,2- a]pyridin-5-yl)- 1-methyl-4- (trifluoromethyl)- 1H-pyrazole- 5-carboxylate | (ES, m/z): [M + H]+ 522.6, ¹H NMR (400 MHz, DMSO) 12.09 (s, 1H), 9.09 (d, J = 2.0 Hz, 1H), 8.86 (d, J = 2.3 Hz, 1H), 8.23 (s, 2H), 7.91 (s, 1H), 7.76 (d, J = 9.1 Hz, 1H), 7.69 (d, J = 1.3 Hz, 1H), 7.43- 7.39 (m, 1H), 7.08 (d, J = 6.8 Hz, 1H), 4.14 (s, 3H). |
| I-224 | 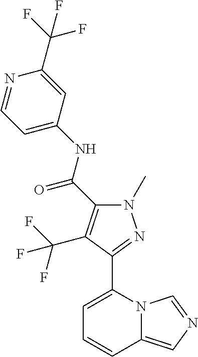 [SMILES: Cn1nc(-c2cccc3cncn23)c(C(F)(F)F)c1C(=O)Nc1ccnc(C(F)(F)F)c1] | methyl 3- (imidazo[1,5- a]pyridin-5-yl)- 1-methyl-4- (trifluoromethyl)- 1H-pyrazole- 5-carboxylate | (ES, m/z): [M + H]+ 455.2, ¹H NMR (400 MHz, DMSO) 8.75 (d, J = 3.8 Hz, 1H), 8.42 (s, 1H), 8.19-8.18 (m, 1H), 7.89-7.85 (m, 1H), 7.76- 7.72 (m, 1H), 7.55 (s, 1H), 6.94 (dd, J = 6.6, 9.1 Hz, 1H), 6.83 (d, J = 6.6 Hz, 1H), 4.09 (s, 3H). |
| I-225 | 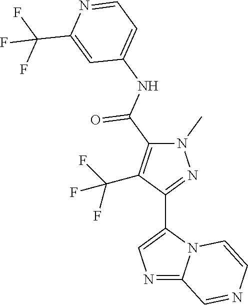 [SMILES: Cn1nc(-c2cnc3cnccn23)c(C(F)(F)F)c1C(=O)Nc1ccnc(C(F)(F)F)c1] | methyl 3- (imidazo[1,2- a]pyrazin-3-yl)- 1-methyl-4- (trifluoromethyl)- 1H-pyrazole- 5-carboxylate | (ES, m/z): [M + H]+ 456.2, ¹H NMR (400 MHz, DMSO) 12.03 (s, 1H), 9.25-9.24 (m, 1H), 9.08 (dd, J = 1.4, 4.7 Hz, 1H), 8.69 (s, 1H), 8.16-8.09 (m, 3H), 7.81 (s, 1H), 4.08 (s, 3H). |
| I-228 | 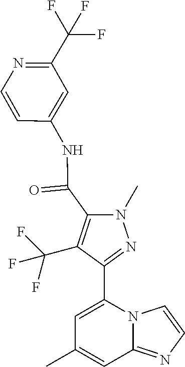 [SMILES: Cc1cc(-c2nn(C)c(C(=O)Nc3ccnc(C(F)(F)F)c3)c2C(F)(F)F)n2ccnc2c1] | methyl 1- methyl-3-(7- methylimidazo[1,2- a]pyridin-5- yl)-4- (trifluoromethyl)- 1H-pyrazole- 5-carboxylate | (ES, m/z): [M + H]+ 469.2, ¹H NMR (400 MHz, DMSO) 11.99 (s, 1H), 8.72 (s, 1H), 8.17 (s, 1H), 7.85 (d, J = 2.3 Hz, 1H), 7.74 (s, 1H), 7.58 (d, J = 1.0 Hz, 1H), 7.52 (s, 1H), 6.88 (s, 1H), 4.09 (s, 3H), 2.42 (s, 3H). |
| I-229 | 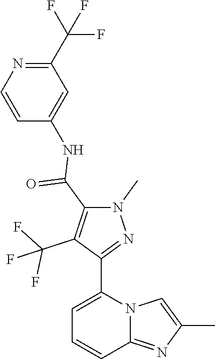 [SMILES: Cc1cn2c(-c3nn(C)c(C(=O)Nc4ccnc(C(F)(F)F)c4)c3C(F)(F)F)cccc2n1] | methyl 1- methyl-3-(2- methylimidazo[1,2- a]pyridin-5- yl)-4- (trifluoromethyl)- 1H-pyrazole- 5-carboxylate | (ES, m/z): [M + H]+ 469.2, ¹H NMR (400 MHz, DMSO) 11.99-11.95 (m, 1H), 8.76 (d, J = 5.1 Hz, 1H), 8.19 (d, J = 1.3 Hz, 1H), 7.87 (d, J = 4.3 Hz, 1H), 7.61 (d, J = 8.3 Hz, 2H), 7.33 (dd, J = 6.9, 9.2 Hz, 1H), 6.97 (d, J = 6.8 Hz, 1H), 4.09 (s, 3H), 2.34 (s, 3H). |
| I-232 | 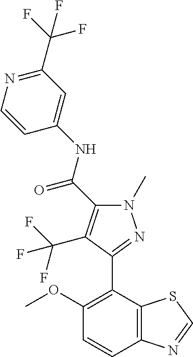 [SMILES: COc1ccc2ncsc2c1-c1nn(C)c(C(=O)Nc2ccnc(C(F)(F)F)c2)c1C(F)(F)F] | methyl 3-(6- methoxybenzo[d] thiazol-7-yl)- 1-methyl-4- (trifluoromethyl)- 1H-pyrazole- 5-carboxylate | (ES, m/z): [M + H]+ 502.2, ¹H NMR (400 MHz, DMSO) 11.95-11.90 (m, 1H), 9.21 (s, 1H), 8.76-8.71 (m, 1H), 8.21-8.14 (m, 2H), 7.94- 7.88 (m, 1H), 7.44 (d, J = 9.1 Hz, 1H), 4.04 (s, 3H), 3.87 (s, 3H). |
| I-238 | 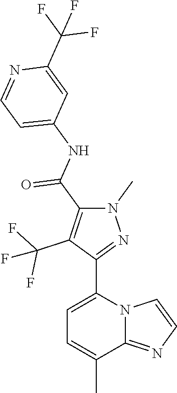 [SMILES: Cc1ccc(-c2nn(C)c(C(=O)Nc3ccnc(C(F)(F)F)c3)c2C(F)(F)F)n2ccnc12] | methyl 1- methyl-3-(8- methylimidazo[1,2- a]pyridin-5- yl)-4- (trifluoromethyl)- 1H-pyrazole- 5-carboxylate | (ES, m/z): [M + H]+ 469.5, ¹H NMR (400 MHz, DMSO) 11.94 (s, 1H), 8.77 (d, J = 5.1 Hz, 1H), 8.19 (d, J = 1.0 Hz, 1H), 7.90-7.87 (m, 2H), 7.64 (d, J = 1.3 Hz, 1H), 7.22 (dd, J = 1.0, 7.1 Hz, 1H), 6.97 (d, J = 7.1 Hz, 1H), 4.09 (s, 3H), 2.57 (s, 3H). |
| I-245 | 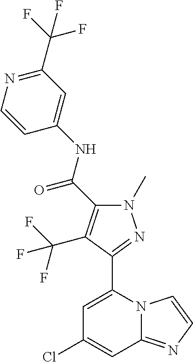 [SMILES: Cn1nc(-c2cc(Cl)cc3nccn23)c(C(F)(F)F)c1C(=O)Nc1ccnc(C(F)(F)F)c1] | methyl 3-(7- chloroimidazo[1,2- a]pyridin-5- yl)-1-methyl-4- (trifluoromethyl)- 1H-pyrazole- 5-carboxylate | (ES, m/z): [M + H]+ 489.5, ¹H NMR (400 MHz, DMSO) 12.04 (s, 1H), 8.69 (s, 1H), 8.15 (s, 1H), 7.96 (d, J = 1.8 Hz, 1H), 7.88 (s, 1H), 7.82 (s, 1H), 7.72 (d, J = 1.0 Hz, 1H), 7.10 (d, J = 2.0 Hz, 1H), 4.10 (s, 3H). |
| I-246 | 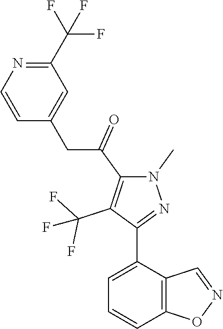 [SMILES: Cn1nc(-c2cccc3oncc23)c(C(F)(F)F)c1C(=O)Cc1ccnc(C(F)(F)F)c1] | methyl 3- (benzo[d]isoxazol- 4-yl)-1- methyl-4- (trifluoromethyl)- 1H-pyrazole- 5-carboxylate | (ES, m/z): [M + H]+ 456.5, ¹H NMR (400 MHz, DMSO) 11.85 (s, 1H), 8.73 (d, J = 5.1 Hz, 1H), 8.17 (d, J = 1.0 Hz, 1H), 7.90-7.87 (m, 1H), 7.58-7.52 (m, 1H), 7.11 (d, J = 8.3 Hz, 1H), 6.89 (d, J = 7.6 Hz, 1H), 4.02 (s, 3H). |
| I-247 | 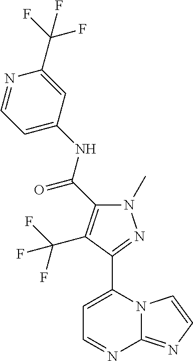 [SMILES: Cn1nc(-c2ccnc3nccn23)c(C(F)(F)F)c1C(=O)Nc1ccnc(C(F)(F)F)c1] | methyl 3- (imidazo[1,2- a]pyrimidin-5- yl)-1-methyl-4- (trifluoromethyl)- 1H-pyrazole- 5-carboxylate | (ES, m/z): [M + H]+ 456.2, ¹H NMR (400 MHz, DMSO) 12.00 (s, 1H), 8.77 (d, J = 5.1 Hz, 1H), 8.72 (d, J = 4.3 Hz, 1H), 8.18 (d, J = 1.5 Hz, 1H), 8.12 (d, J = 1.5 Hz, 1H), 7.89- 7.87 (m, 2H), 7.24 (d, J = 4.3 Hz, 1H), 4.13 (s, 3H). |
| I-248 | 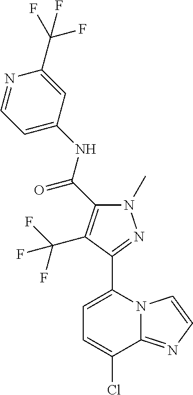 [SMILES: Cn1nc(-c2ccc(Cl)c3nccn23)c(C(F)(F)F)c1C(=O)Nc1ccnc(C(F)(F)F)c1] | methyl 3-(8- chloroimidazo[1,2- a]pyridin-5- yl)-1-methyl-4- (trifluoromethyl)- 1H-pyrazole- 5-carboxylate | (ES, m/z): [M + H]+ 489.2, ¹H NMR (400 MHz, DMSO) 11.99 (s, 1H), 8.74 (d, J = 5.3 Hz, 1H), 8.18 (d, J = 1.3 Hz, 1H), 8.04 (d, J = 1.3 Hz, 1H), 7.86 (d, J = 5.1 Hz, 1H), 7.75 (d, J = 1.3 Hz, 1H), 7.63 (d, J = 7.6 Hz, 1H), 7.07 (d, J = 7.6 Hz, 1H), 4.10 (s, 3H). |
| I-250 | 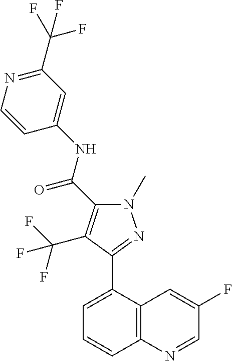 [SMILES: Cn1nc(-c2cccc3ncc(F)cc23)c(C(F)(F)F)c1C(=O)Nc1ccnc(C(F)(F)F)c1] | methyl 3-(3- fluoroquinolin- 5-yl)-1-methyl- 4- (trifluoromethyl)- 1H-pyrazole- 5-carboxylate | (ES, m/z): [M + H]+ 484.2, ¹H NMR (400 MHz, DMSO) 11.90 (s, 1H), 9.06 (d, J = 2.8 Hz, 1H), 8.76 (d, J = 5.3 Hz, 1H), 8.25- 8.19 (m, 2H), 7.97 (dd, J = 2.7, 10.2 Hz, 1H), 7.92-7.87 (m, 2H), 7.75-7.72 (m, 1H), 4.08 (s, 3H). |
| I-292 | 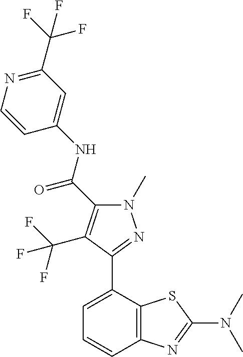 [SMILES: CN(C)c1nc2cccc(-c3nn(C)c(C(=O)Nc4ccnc(C(F)(F)F)c4)c3C(F)(F)F)c2s1] | methyl 3-(2- (dimethylamino) benzo[d]thiazol- 7-yl)-1- methyl-4- (trifluoromethyl)- 1H-pyrazole- 5-carboxylate | (ES, m/z): [M + H]+ 515.2, ¹H NMR (400 MHz, DMSO) 11.96-11.95 (m, 1H), 8.76- 8.70 (m, 1H), 8.17-8.16 (m, 1H), 7.91-7.81 (m, 1H), 7.53 (d, J = 7.8 Hz, 1H), 7.41 (dd, J = 7.7, 7.7 Hz, 1H), 7.21 (d, J = 7.8 Hz, 1H), 4.02 (s, 3H), 3.16 (s, 6H). |
| I-293 | 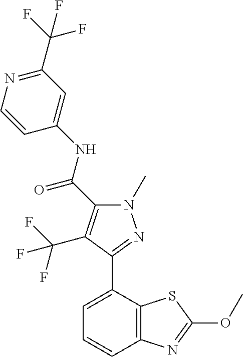 [SMILES: COc1nc2cccc(-c3nn(C)c(C(=O)Nc4ccnc(C(F)(F)F)c4)c3C(F)(F)F)c2s1] | methyl 3-(2- methoxybenzo[d] thiazol-7-yl)- 1-methyl-4- (trifluoromethyl)- 1H-pyrazole- 5-carboxylate | (ES, m/z): [M + H]+ 502.2, ¹H NMR (400 MHz, DMSO) 11.98-11.97 (m, 1H), 8.75 (s, 1H), 8.18 (s, 1H), 7.88 (d, J = 3.0 Hz, 1H), 7.80 (d, J = 7.8 Hz, 1H), 7.58 (dd, J = 8.0, 8.0 Hz, 1H), 7.51 (d, J = 7.3 Hz, 1H), 4.18 (s, 3H), 4.04 (s, 3H). |
| I-294 | 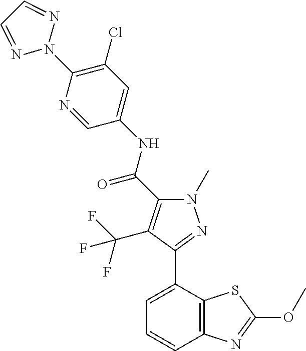 [SMILES: COc1nc2cccc(-c3nn(C)c(C(=O)Nc4cnc(-n5nccn5)c(Cl)c4)c3C(F)(F)F)c2s1] | methyl 3-(2- methoxybenzo[d] thiazol-7-yl)- 1-methyl-4- (trifluoromethyl)- 1H-pyrazole- 5-carboxylate | (ES, m/z): [M + H]+ 535.2, ¹H NMR (400 MHz, DMSO) 11.98 (s, 1H), 8.78 (s, 1H), 8.63 (d, J = 2.3 Hz, 1H), 8.20 (s, 2H), 7.80 (d, J = 7.8 Hz, 1H), 7.59 (dd, J = 7.8, 7.8 Hz, 1H), 7.53 (d, J = 7.8 Hz, 1H), 4.19 (s, 3H), 4.07 (s, 3H). |
| I-295 | 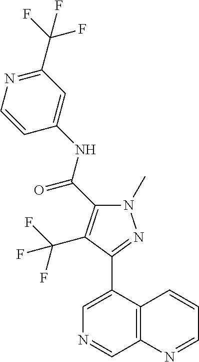 [SMILES: Cn1nc(-c2cncc3ncccc23)c(C(F)(F)F)c1C(=O)Nc1ccnc(C(F)(F)F)c1] | methyl 3-(2- methoxybenzo[d] thiazol-7-yl)- 1-methyl-4- (trifluoromethyl)- 1H-pyrazole- 5-carboxylate | (ES, m/z): [M + H]+ 467.2, ¹H NMR (400 MHz, DMSO) 12.03 (s, 1H), 9.61 (s, 1H), 9.23 (dd, J = 1.5, 4.0 Hz, 1H), 8.81 (d, J = 4.3 Hz, 1H), 8.72 (s, 1H), 8.35 (d, J = 8.3 Hz, 1H), 8.26 (s, 1H), 7.95 (dd, J = 4.3, 8.6 Hz, 2H), 4.16 (s, 3H). |
| I-296 | 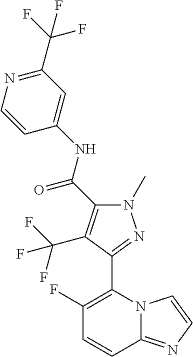 [SMILES: Cn1nc(-c2c(F)ccc3nccn23)c(C(F)(F)F)c1C(=O)Nc1ccnc(C(F)(F)F)c1] | methyl 3-(6- fluoroimidazo[1,2- a]pyridin-5- yl)-1-methyl-4- (trifluoromethyl)- 1H-pyrazole- 5-carboxylate | (ES, m/z): [M + H]+ 473.2, ¹H NMR (400 MHz, DMSO) 12.01-11.98 (m, 1H), 8.77 (d, J = 5.6 Hz, 1H), 8.19 (d, J = 1.8 Hz, 1H), 7.92-7.84 (m, 2H), 7.73 (d, J = 1.8 Hz, 2H), 7.55-7.49 (m, 1H), 4.13 (s, 3H). |
| I-297 | 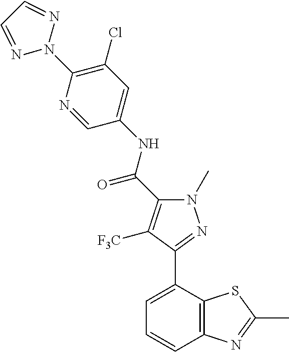 [SMILES: Cc1nc2cccc(-c3nn(C)c(C(=O)Nc4cnc(-n5nccn5)c(Cl)c4)c3C(F)(F)F)c2s1] | methyl 2- methyl-5-(2- methyl-1,3- benzothiazol-7- yl)-4- (trifluoromethyl) pyrazole-3- carboxylate | (ES, m/z): [M + H]+ 519.3; ¹H NMR (400 MHz, CDCl₃) 12.00 (s, 1H), 8.78 (br s, 1H), 8.64 (d, J = 2.2 Hz, 1H), 8.19 (s, 2H), 8.02 (d, J = 7.6 Hz, 1H), 7.65 (t, J = 7.8 Hz, 1H), 7.59 (d, J = 7.4 Hz, 1H), 4.08 (s, 3H), 2.82 (s, 3H). |
| I-298 | 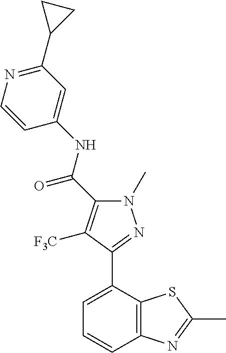 [SMILES: Cc1nc2cccc(-c3nn(C)c(C(=O)Nc4ccnc(C5CC5)c4)c3C(F)(F)F)c2s1] | methyl 2- methyl-5-(2- methyl-1,3- benzothiazol-7- yl)-4- (trifluoromethyl) pyrazole-3- carboxylate | (ES, m/z): [M + H]+ 458.2; ¹H NMR (400 MHz, CDCl₃) 11.49 (s, 1H), 8.36 (d, J = 5.6 Hz, 1H), 8.01 (d, J = 7.3 Hz, 1H), 7.64 (t, J = 7.8 Hz, 1H), 7.62-7.55 (m, 2H), 7.40-7.36 (m, 1H), 4.01 (s, 3H), 2.82 (s, 3H), 2.13-2.03 (m, 1H), 1.26-1.21 (m, 1H), 0.99-0.88 (m, 3H). |

### Example 14-Synthesis of 1-methyl-3-phenyl-4-(trifluoromethyl)-N-(2-(trifluoromethyl)pyridin-4-yl)-1H-pyrazole-5-carboxamide (I-147)

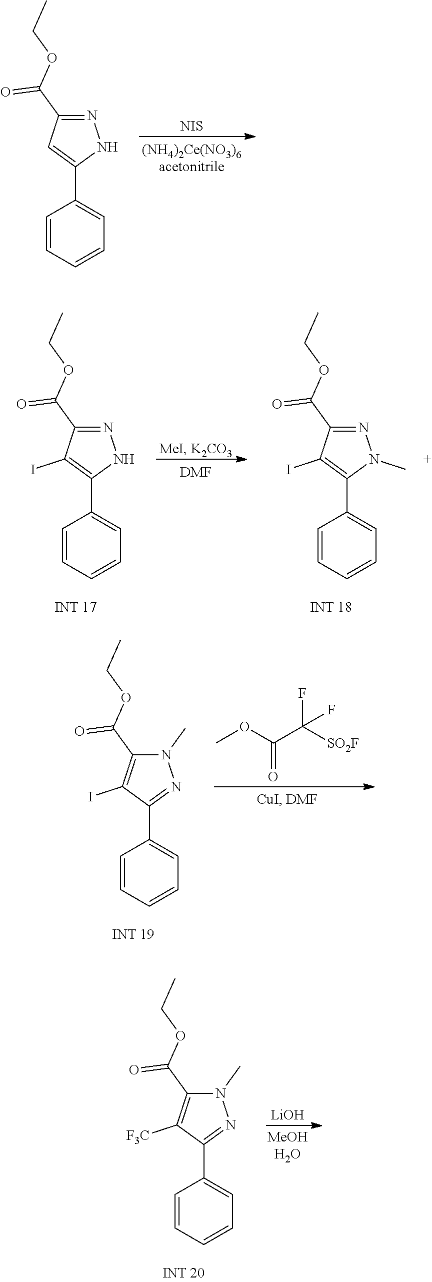

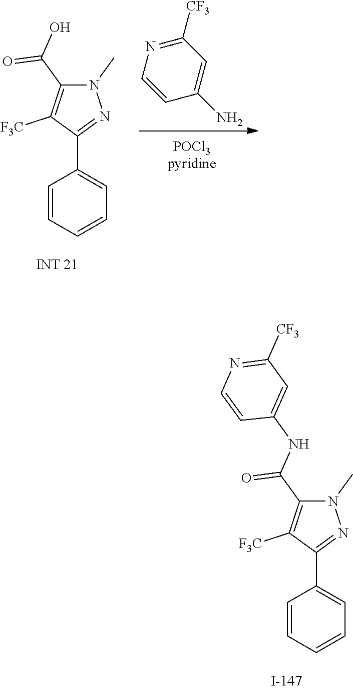

Step 1: Synthesis of ethyl 4-iodo-5-phenyl-1H-pyrazole-3-carboxylate (INT 17): Ceric ammonium nitrate (301 mg, 0.549 mmol, 0.131 eq) was added to a suspension of ethyl 5-phenyl-1H-pyrazole-3-carboxylate (905 mg, 4.19 mmol, 1.00 eq) and NIS (1300 mg, 5.78 mmol, 1.38 eq) in MeCN (15 mL) the reaction was stirred at 70° C. for 2 hours. The solvent was removed under reduced pressure and the residue was partitioned between EtOAc and 1 M aqueous Na₂CO₃. The aqueous phase was separated and extracted with EtOAc. The combined organic phases were dried (MgSO₄) and evaporated to give a brown solid. The crude residue was purified by column chromatography eluting with 10-100% EtOAc in cyclohexane to give the title compound (1.3 g, 3.8 mmol, 91%). ¹H NMR (400 MHz, CDCl₃) δ 11.06 (s, 1H), 7.75-7.71 (m, 2H), 7.50-7.44 (m, 3H), 4.48-4.41 (m, 2H), 1.43 (t, J=3.4 Hz, 3H).

Step 2: Synthesis of ethyl 4-iodo-1-methyl-3-phenyl-1H-pyrazole-5-carboxylate (INT 18): MeI (114 μL, 1.83 mmol, 1.10 eq) was added to a stirred suspension of K₂CO₃ (313 mg, 2.26 mmol, 1.36 eq) and ethyl 4-iodo-5-phenyl-1H-pyrazole-3-carboxylate (569 mg, 1.66 mmol, 1.00 eq) in DMF (5 mL) and the reaction was stirred at rt for 1.5 hours. The solvents were removed under reduced pressure and the residue was partitioned between water and EtOAc. The aqueous phase was separated and extracted with EtOAc. The combined organic phases were dried (MgSO₄) and evaporated. The crude residue was purified by column chromatography eluting with 10-100% EtOAc in cyclohexane to give the title compound INT 18 as a clear oil (385 mg, 1.08 mmol, 65%). ¹H NMR (400 MHz, CDCl₃) δ 7.76-7.73 (m, 2H), 7.47-7.39 (m, 3H), 4.48-4.42 (m, 2H), 4.24 (s, 3H), 1.47 (t, J=7.1 Hz, 3H). Regioisomeric ethyl 4-iodo-1-methyl-5-phenyl-1H-pyrazole-3-carboxylate (INT19) was also isolated by chromatography as a white solid (158 mg, 0.44 mmol, 27%). ¹H NMR (400 MHz, CDCl₃) δ 7.54-7.51 (m, 3H), 7.38-7.35 (m, 2H), 4.47 (q, J=7.1 Hz, 2H), 3.89 (s, 3H), 1.45 (t, J=7.2 Hz, 3H).

Step 3: Synthesis of ethyl 1-methyl-3-phenyl-4-(trifluoromethyl)-1H-pyrazole-5-carboxylate (INT 20): Methyl 2,2-difluoro-2-(fluorosulfonyl)acetate (153 μL, 1.20 mmol, 2.00 eq) was added to a stirred suspension of CuI (229 mg, 1.20 mmol, 2.00 eq) and ethyl 4-iodo-1-methyl-3-phenyl-1H-pyrazole-5-carboxylate (214 mg, 0.601 mmol, 1.00 eq) in DMF (2 mL). The reaction mixture was stirred at 90° C. for 2 hours. The reaction was quenched with saturated aqueous NaHCO₃ and EtOAc was added. The mixture was filtered through Celite® and the filtrate was concentrated, re-dissolved in EtOAc and washed with water. The aqueous phase was separated and extracted with EtOAc and the combined organic layers were dried (MgSO₄) and evaporated. The crude residue was purified by column chromatography eluting with 10-50% EtOAc in cyclohexane to give the title compound as a clear oil (100 mg, 0.3 mmol, 55%). ¹H NMR (400 MHz, CDCl₃) δ 7.55-7.51 (m, 2H), 7.43-7.40 (m, 3H), 4.44 (q, J=7.2 Hz, 2H), 4.15 (s, 3H), 1.42 (t, J=7.2 Hz, 3H).

Step 4: Synthesis of 1-methyl-3-phenyl-4-(trifluoromethyl)-1H-pyrazole-5-carboxylic acid (INT 21): LiOH (17 mg, 0.410 mmol, 1.22 eq) was added to a solution of ethyl 1-methyl-3-phenyl-4-(trifluoromethyl)-1H-pyrazole-5-carboxylate (100 mg, 0.335 mmol, 1.00 eq) in MeOH (1.50 mL) and water (0.20 mL) and the reaction was stirred at 40° C. for 3 hours. The solvents were evaporated under reduced pressure and the residue was used without further purification.

Step 5: Synthesis of 1-methyl-3-phenyl-4-(trifluoromethyl)-N-(2-(trifluoromethyl)pyridin-4-yl)-1H-pyrazole-5-carboxamide (I-147): POCl₃ (31 μL, 0.333 mmol, 2.00 eq) was added to a solution of 1-methyl-3-phenyl-4-(trifluoromethyl)-1H-pyrazole-5-carboxylic acid (45 mg, 0.167 mmol, 1.00 eq) and 4-amino-2-(trifluoromethyl)pyridine (35 mg, 0.216 mmol, 1.30 eq) in pyridine (1.5 mL). The reaction was stirred at rt for 1.5 h, then a further portion of POCl₃ (31 μL, 0.333 mmol, 2.00 eq) was added. After a further 1 hour, the reaction was quenched with 1 M aqueous Na₂CO₃ and extracted with EtOAc. The organic phase was dried (MgSO₄) and evaporated. The crude residue was successively purified by column chromatography eluting with 5-100% EtOAc in cyclohexane and by reverse phase HPLC to give the title compound (8.7 mg, 0.02 mmol, 12%). (ES, m/z): [M+H]⁺ 415.5, ¹H NMR (400 MHz, DMSO) δ 11.97 (s, 1H), 8.75 (d, J=5.5 Hz, 1H), 8.19 (d, J=1.5 Hz, 1H), 7.88 (dd, J=0.4, 3.7 Hz, 1H), 7.61 (dd, J=1.7, 7.8 Hz, 2H), 7.56-7.49 (m, 3H), 4.00 (s, 3H).

### Example 15—Synthesis of Additional Compounds

Table 10 shows exemplary compounds synthesized from ethyl 4-iodo-1-methyl-3-phenyl-1H-pyrazole-5-carboxylate using the route described in the prior Example.

TABLE 10

| Compound | | Starting | |
|---|---|---|---|
| No. | Structure | Material | Analytical data |
| I-165 | 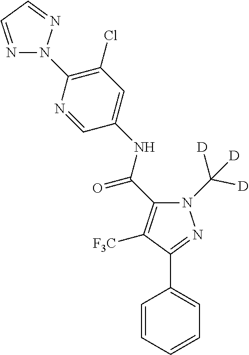 [SMILES: [2H]C([2H])([2H])n1nc(-c2ccccc2)c(C(F)(F)F)c1C(=O)Nc1cnc(-n2nccn2)c(Cl)c1] | MeI-d₃ and 5-chloro-6- (2H-1,2,3- triazol-2- yl)pyridin-3- amine | (ES, m/z): [M + H]⁺ 451.3, ¹H NMR (400 MHz, DMSO) δ 11.91 (s, 1H), 8.74 (s, 1H), 8.62 (d, J = 2.3 Hz, 1H), 8.18 (s, 2H), 7.60 (dd, J = 1.6, 7.8 Hz, 2H), 7.55- 7.48 (m, 3H) |
| I-166 | 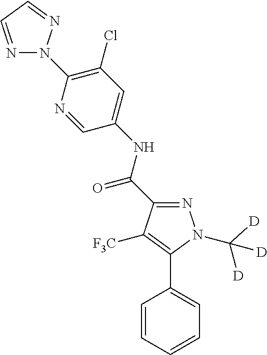 [SMILES: [2H]C([2H])([2H])n1nc(C(=O)Nc2cnc(-n3nccn3)c(Cl)c2)c(C(F)(F)F)c1-c1ccccc1] | MeI-d₃ and 5-chloro-6- (2H-1,2,3- triazol-2- yl)pyridin-3- amine | (ES, m/z): [M + H]⁺ 451.4, ¹H NMR (400 MHz, DMSO) δ 11.24 (s, 1H), 8.95 (d, J = 1.8 Hz, 1H), 8.74-8.73 (m, 1H), 8.18 (s, 2H), 7.61-7.54 (m, 5H) |
| I-167 | 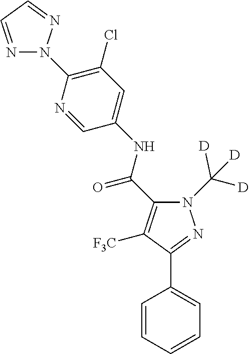 [SMILES: [2H]C([2H])([2H])n1nc(-c2ccccc2)c(C(F)(F)F)c1C(=O)Nc1cnc(-n2nccn2)c(Cl)c1] | MeI-d₃ and 5-amino-2- (2H-1,2,3- triazol-2- yl)nicotino- nitrile | (ES, m/z): [M + H]⁺ 442.3, ¹H NMR (400 MHz, DMSO) δ 11.95 (s, 1H), 9.03 (d, J = 2.5 Hz, 1H), 8.82 (d, J = 2.5 Hz, 1H), 8.32 (s, 2H), 7.62-7.58 (m, 2H), 7.55- 7.48 (m, 3H) |
| I-168 | 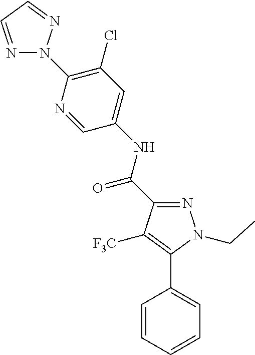 [SMILES: CCn1nc(C(=O)Nc2cnc(-n3nccn3)c(Cl)c2)c(C(F)(F)F)c1-c1ccccc1] | Iodoethane and 5-chloro- 6-(2H-1,2,3- triazol-2- yl)pyridin-3- amine | (ES, m/z): [M + H]⁺ 462.2, ¹H NMR (400 MHz, DMSO) δ 11.16 (s, 1H), 8.99 (d, J = 2.3 Hz, 1H), 8.74 (d, J = 2.0 Hz, 1H), 8.18 (s, 2H), 7.62-7.57 (m, 3H), 7.54- 7.50 (m, 2H), 4.05 (q, J = 7.2 Hz, 2H), 1.32 (t, J = 7.2 Hz, 3H) |
| I-169 | 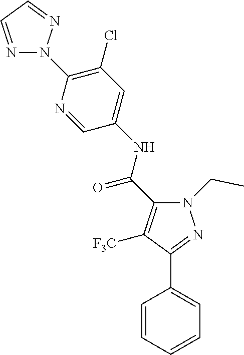 | iodoethane and 5-amino- 2-(2H-1,2,3- triazol-2- yl)nicotino- nitrile | (ES, m/z): [M + H]+ 451.2, ¹H NMR (400 MHz, DMSO) δ 12.00 (s, 1H), 9.01 (s, 1H), 8.82 (d, J = 2.5 Hz, IH), 8.32 (s, 2H), 7.62 (dd, J = 1.4, 7.7 Hz, 2H), 7.55- 7.47 (m, 3H), 4.32 (q, J = 7.2 Hz, 2H), 1.45 (t, J = 7.2 Hz, 3H) |
| I-170 | 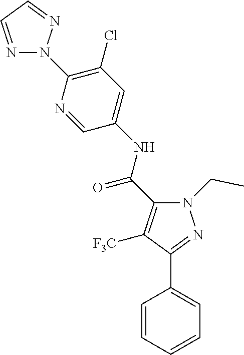 [SMILES: CCn1nc(-c2ccccc2)c(C(F)(F)F)c1C(=O)Nc1cnc(-n2nccn2)c(Cl)c1] | iodoethane and 5-chloro- 6-(2H-1,2,3- triazol-2- yl)pyridin-3- amine | (ES, m/z): [M + H]⁺ 462.2, ¹H NMR (400 MHz, DMSO) δ 11.95 (s, 1H), 8.77 (s, 1H), 8.62 (d, J = 2.3 Hz, 1H), 8.19 (s, 2H), 7.62 (d, J = 6.3 Hz, 2H), 7.55-7.48 (m, 3H), 4.32 (q, J = 7.2 Hz, 2H), 1.45 (t, J = 7.2 Hz, 3H) |

### Example 16-Synthesis of N-(5-chloro-6-(2H-1,2,3-triazol-2-yl) pyridin-3-yl)-1-(2-(dimethylamino)ethyl)-3-phenyl-4-(trifluoromethyl)-1H-pyrazole-5-carboxamide (I-171)

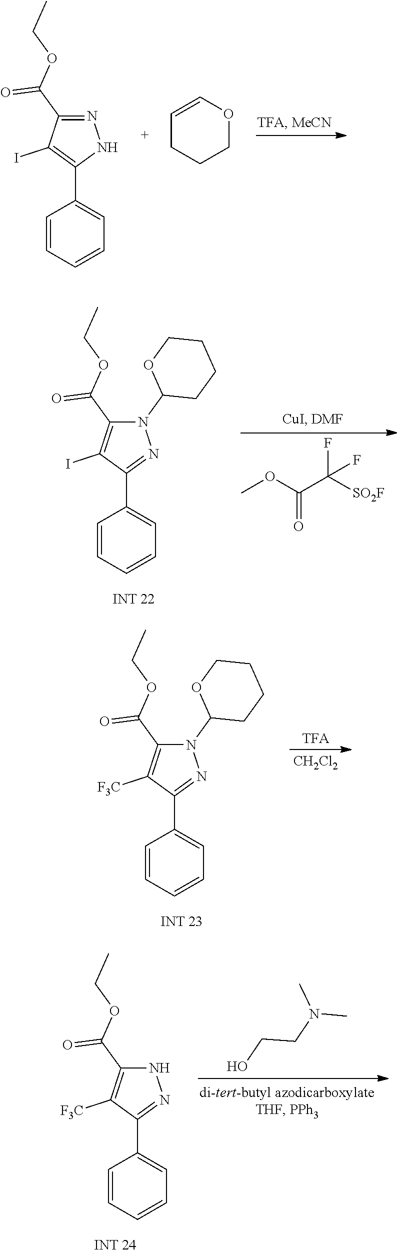

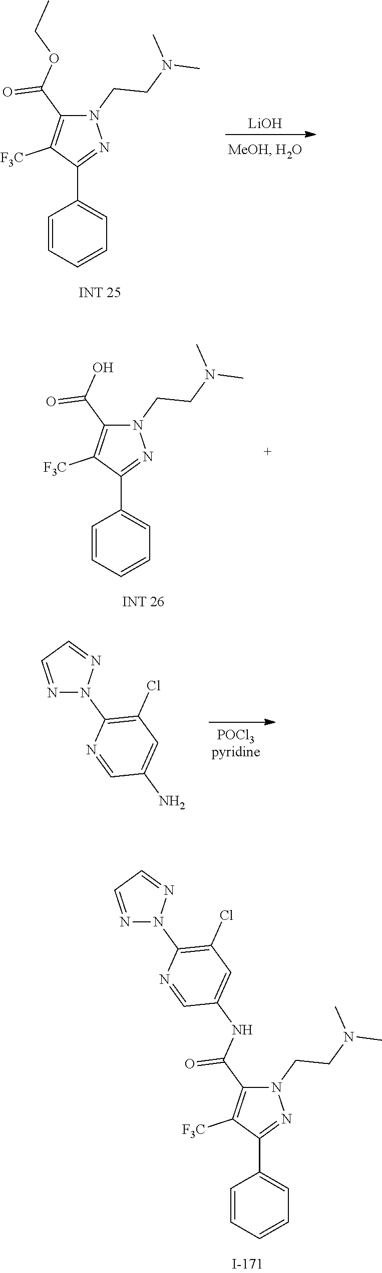 [SMILES: CCOC(=O)c1c(C(F)(F)F)c(-c2ccccc2)nn1CCN(C)C ; "LiOH" ; CN(C)CCn1nc(-c2ccccc2)c(C(F)(F)F)c1C(=O)O ; Nc1cnc(-n2nccn2)c(Cl)c1 ; CN(C)CCn1nc(-c2ccccc2)c(C(F)(F)F)c1C(=O)Nc1cnc(-n2nccn2)c(Cl)c1]

Step 1: Synthesis of ethyl 4-iodo-3-phenyl-1-(tetrahydro-2H-pyran-2-yl)-1H-pyrazole-5-carboxylate (INT 22): TFA (5.1 μL, 0.07 mmol, 0.02 eq) was added to a solution of ethyl 4-iodo-5-phenyl-1H-pyrazole-3-carboxylate (1.15 g, 3.36 mmol, 1.00 eq) and 3,4-dihydro-2H-pyran (613 μL, 6.72 mmol, 2.00 eq) in MeCN (20 mL) and the reaction was refluxed for 2 hours. The solvent was removed under reduced pressure and the crude residue was partitioned between EtOAc and 1.8 M aqueous K₂CO₃, the organic phase was separated, dried (MgSO₄) and evaporated. The crude residue was purified by column chromatography eluting with 5-40% EtOAc in cyclohexane to give the title compound (862 mg 1.94 mmol, 58%) as a clear glass. ¹H NMR (400 MHz, CDCl₃) δ 7.53-7.46 (m, 5H), 5.15-5.11 (m, 1H), 4.48-4.42 (m, 2H), 4.09-4.03 (m, 1H), 3.50-3.42 (m, 1H), 2.57-2.46 (m, 1H), 2.07-1.99 (m, 1H), 1.84-1.65 (m, 2H), 1.56-1.47 (m, 2H), 1.43 (t, J=7.1 Hz, 3H).

Step 2: Synthesis of ethyl 3-phenyl-1-(tetrahydro-2H-pyran-2-yl)-4-(trifluoromethyl)-1H-pyrazole-5-carboxylate (INT 23): Methyl 2,2-difluoro-2-(fluorosulfonyl)acetate (240 μL, 1.89 mmol, 2.01 eq) was added to a stirred suspension of copper (I) iodide (370 mg, 1.94 mmol, 2.07 eq) and ethyl 4-iodo-3-phenyl-1-(tetrahydro-2H-pyran-2-yl)-1H-pyrazole-5-carboxylate (400 mg, 0.938 mmol, 1.00 eq) in DMF (4 mL) and the reaction was stirred at 90° C. for 2 hours. The reaction was quenched with saturated aqueous NaHCO₃ and diluted with EtOAc. The biphasic mixture was filtered through a pad of Celite®. The aqueous layer was separated and extracted with EtOAc and the combined organic phases were dried (MgSO₄) and evaporated. The crude residue was purified by column chromatography eluting with 5-30% EtOAc in cyclohexane to give the title compound as a clear oil (267 mg, 0.73 mmol, 77%). ¹H NMR (400 MHz, CDCl₃) δ 7.54-7.41 (m, 5H), 4.96 (dd, J=2.6, 10.2 Hz, 1H), 4.47-4.41 (m, 2H), 4.06-4.00 (m, 1H), 3.42-3.35 (m, 1H), 2.59-2.47 (m, 1H), 2.07-2.00 (m, 1H), 1.83-1.77 (m, 1H), 1.73-1.64 (m, 1H), 1.52-1.45 (m, 2H), 1.40 (t, J=7.2 Hz, 3H).

Step 3: Synthesis of ethyl 3-phenyl-4-(trifluoromethyl)-1H-pyrazole-5-carboxylate (INT 24): 4 M HCl in 1,4-dioxane (906 μL, 3.62 mmol, 5.00 eq) was added to a stirred solution of ethyl 3-phenyl-1-(tetrahydro-2H-pyran-2-yl)-4-(trifluoromethyl)-1H-pyrazole-5-carboxylate (267 mg, 0.725 mmol, 1.00 eq) in EtOH (5 mL) and the reaction was stirred at 65° C. for 1 hour. The solvent was removed under reduced pressure and the residue was partitioned between EtOAc and 1.8 M aqueous K₂CO₃. The layers were separated and the aqueous one was extracted with EtOAc. The combined organic layers were dried (MgSO₄) and evaporated. The crude residue was purified by column chromatography eluting with 5-50% EtOAc in cyclohexane to give the title compound (155 mg, 0.54 mmol, 75%) as a white solid. ¹H NMR (400 MHz, CDCl₃) δ 11.48 (1H, s), 7.48-7.46 (5H, m), 4.41-4.35 (2H, m), 1.36 (3H, t, J=7.1 Hz).

Step 4: Synthesis of ethyl 1-(2-(dimethylamino)ethyl)-3-phenyl-4-(trifluoromethyl)-1H-pyrazole-5-carboxylate (INT 25): di-tert-Butyl azodicarboxylate (160 mg, 0.695 mmol, 1.22 eq) was added portionwise over 10 minutes to an ice-cooled mixture of ethyl 3-phenyl-4-(trifluoromethyl)-1H-pyrazole-5-carboxylate (162 mg, 0.570 mmol, 1.00 eq), PPh₃ (180 mg, 0.686 mmol, 1.20 eq) and 2-dimethylaminoethanol (69 μL, 0.686 mmol, 1.20 eq) in THF (3 mL). The reaction mixture was stirred at rt for 4 hours. The solvent was removed under reduced pressure and the residue was successively purified by column chromatography eluting with 10-100% EtOAc in cyclohexane and by SCX eluting with 3.5 N NH₃ in MeOH, to give the title compound as a clear oil (131 mg, 0.37 mmol, 65%), which contained 15% ethyl 1-(2-(dimethylamino)ethyl)-5-phenyl-4-(trifluoromethyl)-1H-pyrazole-3-carboxylate. ¹H NMR (400 MHz, CDCl₃) δ 7.57-7.54 (m, 2H), 7.44-7.40 (m, 3H), 4.58-4.54 (m, 2H), 4.43 (q, J=7.2 Hz, 2H), 2.75 (t, J=6.6 Hz, 2H), 2.27 (s, 6H), 1.44-1.40 (m, 3H).

Step 5: Synthesis of 1-(2-(dimethylamino)ethyl)-3-phenyl-4-(trifluoromethyl)-1H-pyrazole-5-carboxylic acid (INT 26): LiOH·H₂O (20 mg, 0.469 mmol, 1.27 eq) was added to a solution of ethyl 1-(2-(dimethylamino)ethyl)-3-phenyl-4-(trifluoromethyl)-1H-pyrazole-5-carboxylate (131 mg, 0.369 mmol, 1.00 eq) in MeOH (1 mL) and water (0.20 mL), the reaction was stirred at 40° C. for 4 hours. The solvent was removed under reduced pressure to give the title compound, which was used without further purification.

Step 6: Synthesis of N-(5-chloro-6-(2H-1,2,3-triazol-2-yl) pyridin-3-yl)-1-(2-(dimethylamino)ethyl)-3-phenyl-4-(trifluoromethyl)-1H-pyrazole-5-carboxamide (I-171): POCl₃ (36 μL, 0.386 mmol, 2.14 eq) was added to a solution of 5-chloro-6-(triazol-2-yl) pyridin-3-amine (40 mg, 0.204 mmol, 1.13 eq) and 1-(2-(dimethylamino)ethyl)-3-phenyl-4-(trifluoromethyl)-1H-pyrazole-5-carboxylic acid (59 mg, 0.18 mmol, 1.00 eq) in pyridine (1 mL) and the reaction was stirred at rt for 2.5 hours. The reaction was quenched with 1 M aqueous K₂CO₃ and extracted with EtOAc. The combined organic phases were dried (MgSO₄) and the residue was successively purified by column chromatography eluting with 10-100% EtOAc in cyclohexane and by reverse phase HPLC to give the title compound (20.7 mg, 0.04 mmol, 22%). (ES, m/z): [M+H]⁺ 505.5, ¹H NMR (400 MHz, DMSO) δ 8.83 (d, J=2.3 Hz, 1H), 8.64 (d, J=2.3 Hz, 1H), 8.22 (s, 2H), 7.63 (dd, J=1.4, 7.7 Hz, 2H), 7.58-7.51 (m, 3H), 4.41 (t, J=6.0 Hz, 2H), 2.76 (t, J=5.5 Hz, 2H), 2.17 (s, 6H), NH not seen.

### Example 17—Synthesis of Additional Compounds

Compounds in Table 11 were synthesized from ethyl 3-phenyl-4-(trifluoromethyl)-1H-pyrazole-5-carboxylate using the route described above.

TABLE 11

| Compound | | Starting | |
|---|---|---|---|
| No. | Structure | Material | Analytical data |
| I-172 | 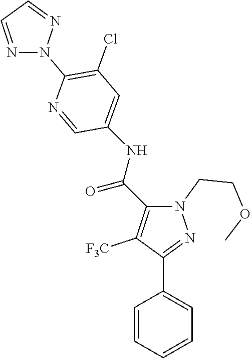 [SMILES: COCCn1nc(-c2ccccc2)c(C(F)(F)F)c1C(=O)Nc1cnc(-n2nccn2)c(Cl)c1] | 2- Methoxyethanol and 5-chloro- 6-(2H-1,2,3- triazol-2- yl)pyridin-3- amine | (ES, m/z): [M + H]⁺ 492.5, ¹H NMR (400 MHz, DMSO) δ 11.89 (s, 1H), 8.80 (s, 1H), 8.63 (d, J = 2.3 Hz, 1H), 8.21 (s, 2H), 7.65-7.61 (m, 2H), 7.58-7.50 (m, 3H), 4.50 (t, J = 5.1 Hz, 2H), 3.76 (t, J = 5.1 Hz, 2H), 3.23 (s, 3H) |
| I-173 | 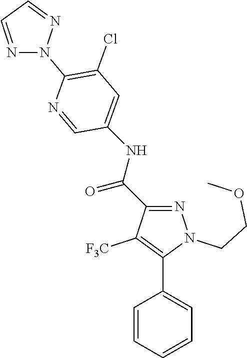 [SMILES: COCCn1nc(C(=O)Nc2cnc(-n3nccn3)c(Cl)c2)c(C(F)(F)F)c1-c1ccccc1] | 2- Methoxyethanol and 5-chloro- 6-(2H-1,2,3- triazol-2- yl)pyridin-3- amine | (ES, m/z): [M + H]⁺ 492.2, ¹H NMR (400 MHz, DMSO) δ 11.20 (s, 1H), 9.00 (d, J = 2.3 Hz, 1H), 8.75 (d, J = 2.3 Hz, 1H), 8.20 (s, 2H), 7.62-7.58 (m, 3H), 7.53-7.49 (m, 2H), 4.19 (t, J = 5.2 Hz, 2H), 3.72 (t, J = 5.2 Hz, 2H), 3.14 (s, 3H) |
| I-174 | 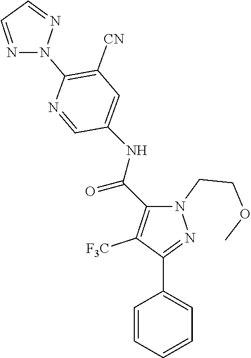 [SMILES: COCCn1nc(-c2ccccc2)c(C(F)(F)F)c1C(=O)Nc1cnc(-n2nccn2)c(C#N)c1] | 2- Methoxyethanol and 5-amino- 2-(2H-1,2,3- triazol-2- yl)nicotinonitrile | (ES, m/z): [M + H]⁺ 483.3, ¹H NMR (400 MHz, DMSO) δ 11.94 (s, 1H), 9.05-9.03 (m, 1H), 8.86-8.82 (m, 1H), 8.34- 8.33 (m, 2H), 7.64 (d, J = 8.3 Hz, 2H), 7.57-7.50 (m, 3H), 4.50 (t, J = 5.0 Hz, 2H), 3.78-3.74 (m, 2H), 3.23 (s, 3H) |
| I-175 | 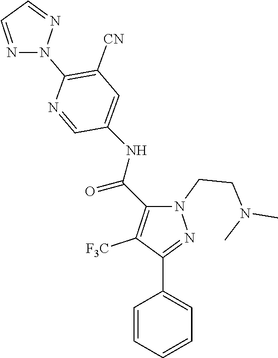 [SMILES: CN(C)CCn1nc(-c2ccccc2)c(C(F)(F)F)c1C(=O)Nc1cnc(-n2nccn2)c(C#N)c1] | 2- Dimethylamino ethanol and 5- amino-2-(2H- 1,2,3-triazol-2- yl)nicotinonitrile | (ES, m/z): [M + H]⁺ 496.2, ¹H NMR (400 MHz, DMSO) δ 9.05 (d, J = 2.1 Hz, 1H), 8.86-8.84 (m, 1H), 8.34 (s, 2H), 7.63 (dd, J = 1.5, 7.9 Hz, 2H), 7.57-7.49 (m, 3H), 4.43-4.38 (m, 2H), 2.77-2.72 (m, 2H), 2.14 (s, 6H), NH not seen |
| I-176 | 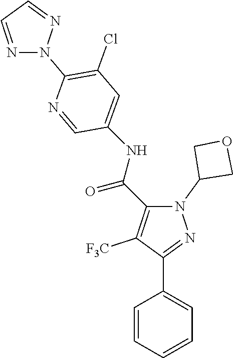 [SMILES: O=C(Nc1cnc(-n2nccn2)c(Cl)c1)c1c(C(F)(F)F)c(-c2ccccc2)nn1C1COC1] | oxetan-3-ol and N-(5-chloro-6- (2H-1,2,3- triazol-2- yl)pyridin-3- yl)-1-(2- methoxyethyl)- 5-phenyl-4- (trifluoromethyl)- 1H-pyrazole- 3-carboxamide | (ES, m/z): [M + H]⁺ 490.3, ¹H NMR (400 MHz, DMSO) δ 11.97 (s, 1H), 8.70 (s, 1H), 8.60 (d, J = 2.3 Hz, 1H), 8.19 (s, 2H), 7.68 (dd, J = 1.5, 7.8 Hz, 2H), 7.57-7.53 (m, 3H), 5.90-5.82 (m, 1H), 5.05 (t, J = 6.5 Hz, 2H), 4.94 (t, J = 7.3 Hz, 2H) |
| I-177 | 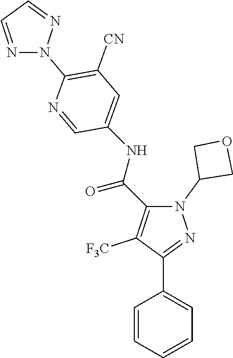 [SMILES: N#Cc1cc(NC(=O)c2c(C(F)(F)F)c(-c3ccccc3)nn2C2COC2)cnc1-n1nccn1] | oxetan-3-ol and 5-amino-2-(2H- 1,2,3-triazol-2- yl)nicotinonitrile | (ES, m/z): [M + H]⁺ 481.3, ¹H NMR (400 MHz, DMSO) δ 11.96 (s, 1H), 8.98 (s, 1H), 8.80 (d, J = 2.6 Hz, 1H), 8.32 (s, 2H), 7.70-7.66 (m, 2H), 7.57-7.53 (m, 3H), 5.85 (tt, J = 6.8, 6.8 Hz, 1H), 5.05 (t, J = 6.4 Hz, 2H), 4.94 (t, J = 7.3 Hz, 2H) |
| I-178 | 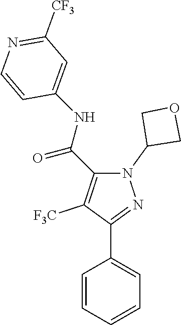 [SMILES: O=C(Nc1ccnc(C(F)(F)F)c1)c1c(C(F)(F)F)c(-c2ccccc2)nn1C1COC1] | oxetan-3-ol and 2- (trifluoromethyl) pyridin-4- amine | (ES, m/z): [M + H]⁺ 457.5, ¹H NMR (400 MHz, DMSO) δ 11.90 (s, 1H), 8.74 (s, 1H), 8.17 (s, 1H), 7.82-7.80 (m, 1H), 7.67 (d, J = 6.3 Hz, 2H), 7.56- 7.52 (m, 3H), 5.84-5.76 (m, 1H), 5.03 (t, J = 6.4 Hz, 2H), 4.92 (t, J = 7.2 Hz, 2H) |

### Example 18—Synthesis of N-(5-chloro-6-(2H-1,2,3-triazol-2-yl) pyridin-3-yl)-1-(2-hydroxyethyl)-3-phenyl-4-(trifluoromethyl)-1H-pyrazole-5-carboxamide (I-211)

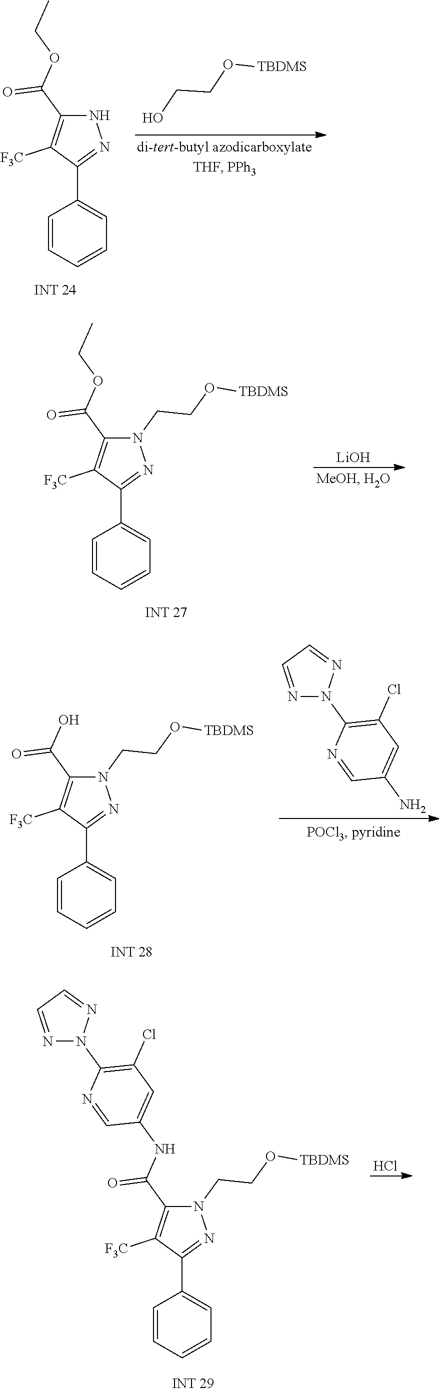

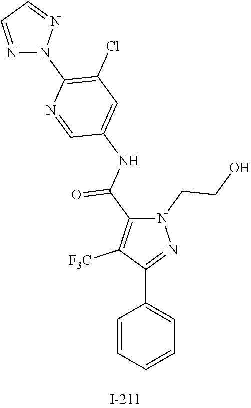 [SMILES: O=C(Nc1cnc(-n2nccn2)c(Cl)c1)c1c(C(F)(F)F)c(-c2ccccc2)nn1CCO]

Step 1: Synthesis of ethyl 1-(2-((tert-butyldimethylsilyl)oxy)ethyl)-3-phenyl-4-(trifluoromethyl)-1H-pyrazole-5-carboxylate (INT 27): di-tert-Butyl azodicarboxylate (160 mg, 0.695 mmol, 1.23 eq) was added in portions over 15 minutes to an ice-cooled solution of ethyl 3-phenyl-4-(trifluoromethyl)-1H-pyrazole-5-carboxylate (160 mg, 0.563 mmol, 1.00 eq), PPh₃ (195 mg, 0.744 mmol, 1.32 eq) and 2-[tert-butyl(dimethyl)silyl]oxyethanol (121 mg, 0.685 mmol, 1.22 eq) in THF (4 mL), the reaction was stirred at rt for 16 hours. The reaction mixture was concentrated under reduced pressure and the crude residue was purified by column chromatography eluting with 5-50% EtOAc in cyclohexane to give the title compound as clear oil (54 mg, 0.12 mmol, 22%). ¹H NMR (400 MHz, CDCl₃) δ 7.55-7.51 (m, 2H), 7.44-7.40 (m, 3H), 4.64 (t, J=5.4 Hz, 2H), 4.42 (q, J=7.2 Hz, 2H), 3.96 (t, J=5.4 Hz, 2H), 1.43-1.38 (m, 3H), 0.80 (s, 9H), −0.08 (s, 6H).

Step 2: Synthesis of 1-(2-((tert-butyldimethylsilyl)oxy)ethyl)-3-phenyl-4-(trifluoromethyl)-1H-pyrazole-5-carboxylic acid (INT 28): LiOH·H₂O (6.1 mg, 0.146 mmol, 1.20 eq) was added to a solution of ethyl 1-(2-((tert-butyldimethylsilyl)oxy)ethyl)-3-phenyl-4-(trifluoromethyl)-1H-pyrazole-5-carboxylate (54 mg, 0.122 mmol, 1.00 eq) in MeOH (1 mL) and water (0.20 mL), the reaction was stirred at 40° C. for 2 hours. The solvent was removed under reduced pressure to give the title compound, which was used in the next step without further purification.

Step 3: Synthesis of 1-(2-((tert-butyldimethylsilyl)oxy)ethyl)-N-(5-chloro-6-(2H-1,2,3-triazol-2-yl) pyridin-3-yl)-3-phenyl-4-(trifluoromethyl)-1H-pyrazole-5-carboxamide (INT 29): POCl₃ (16 μL, 0.172 mmol, 1.19 eq) was added to a solution of 5-chloro-6-(triazol-2-yl) pyridin-3-amine (33 mg, 0.167 mmol, 1.15 eq) and 1-(2-((tert-butyldimethylsilyl)oxy)ethyl)-3-phenyl-4-(trifluoromethyl)-1H-pyrazole-5-carboxylic acid (60 mg, 0.145 mmol, 1.00 eq) in pyridine (1.5 mL) and the reaction was stirred at rt for 2.5 hours. The reaction was quenched with 1 M aqueous K₂CO₃ and extracted with EtOAc. The combined organic extracts were dried (MgSO₄) and evaporated. The crude residue was purified by column chromatography 5-100% EtOAc in cyclohexane to give the title compound (24 mg, 0.038 mmol, 26%). ¹H NMR (400 MHz, CDCl₃) δ 10.14 (s, 1H), 8.86 (d, J=2.4 Hz, 1H), 8.51 (d, J=2.4 Hz, 1H), 7.96 (s, 2H), 7.63-7.57 (m, 2H), 7.48-7.45 (m, 3H), 4.58-4.54 (m, 2H), 4.29 (t, J=4.9 Hz, 2H), 0.85-0.84 (m, 9H), 0.07 (s, 6H).

Step 4: Synthesis of N-(5-chloro-6-(2H-1,2,3-triazol-2-yl) pyridin-3-yl)-1-(2-hydroxyethyl)-3-phenyl-4-(trifluoromethyl)-1H-pyrazole-5-carboxamide (1-211): 12 M aqueous HCl (20 μL, 0.240 mmol, 5.92 eq) was added to a solution of 1-(2-((tert-butyldimethylsilyl)oxy)ethyl)-N-(5-chloro-6-(2H-1,2,3-triazol-2-yl) pyridin-3-yl)-3-phenyl-4-(trifluoromethyl)-1H-pyrazole-5-carboxamide (24 mg, 0.0405 mmol, 1.00 eq) in MeOH (2 mL), the mixture was stirred at rt for 1 hour. K₂CO₃ (34 mg, 0.244 mmol, 6.02 eq) was added and the mixture was stirred for 1 hour. The suspension was filtered, washed with MeOH and the filtrate was concentrated under reduced pressure. The crude residue was purified by reverse phase HPLC to give the title compound as an off-white solid (2.71 mg, 0.0056 mmol, 5%). (ES, m/z): [M+H]⁺ 478.2, ¹H NMR (400 MHz, DMSO) δ 11.87 (s, 1H), 8.78 (d, J=2.0 Hz, 1H), 8.60 (d, J=2.0 Hz, 1H), 8.19 (s, 2H), 7.63-7.60 (m, 2H), 7.55-7.49 (m, 3H), 5.14 (s, 1H), 4.36 (t, J=5.3 Hz, 2H), 3.80 (t, J=5.2 Hz, 2H).

### Example 19—Synthesis of N-(5-chloro-6-(2H-1,2,3-triazol-2-yl) pyridin-3-yl)-1-(difluoromethyl)-3-phenyl-4-(trifluoromethyl)-1H-pyrazole-5-carboxamide (I-179)

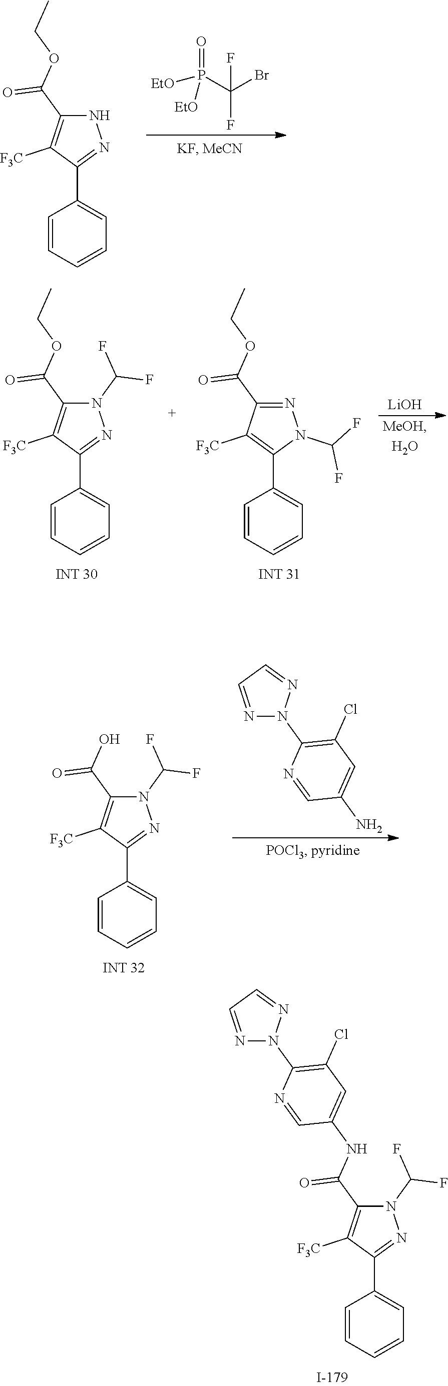

Step 1: Synthesis of ethyl 1-(difluoromethyl)-3-phenyl-4-(trifluoromethyl)-1H-pyrazole-5-carboxylate (INT 30): Diethyl (bromodifluoromethyl)phosphonate (115 μL, 0.647 mmol, 1.11 eq) was added to a solution of ethyl 3-phenyl-4-(trifluoromethyl)-1H-pyrazole-5-carboxylate (166 mg, 0.584 mmol, 1.00 eq) and KF (72 mg, 1.24 mmol, 2.12 eq) in MeCN (3 mL). The reaction was stirred at rt for 1.5 hours. K₂CO₃ (165 mg, 1.20 mmol, 2.05 eq) was added and the mixture was diluted with EtOAc (10 mL). The suspension was filtered through Celite® and the solvent was evaporated under reduced pressure. The crude residue was purified by column chromatography eluting with 5-50% EtOAc in cyclohexane to give the title compound (35 mg, 0.11 mmol, 18%). ¹H NMR (400 MHz, CDCl₃) δ 7.73 (t, J=56.8 Hz, 1H), 7.60-7.55 (m, 2H), 7.48-7.39 (m, 3H), 4.50 (q, J=7.0 Hz, 2H), 1.44 (t, J=7.0 Hz, 3H). Regioisomeric ethyl 1-(difluoromethyl)-5-phenyl-4-(trifluoromethyl)-1H-pyrazole-3-carboxylate was also isolated by chromatography (INT 31) (81 mg, 0.24 mmol, 41%). ¹H NMR (400 MHz, CDCl₃) δ 7.59-7.49 (m, 3H), 7.41 (d, J=7.6 Hz, 2H), 7.05 (t, J=57.7 Hz, 1H), 4.49 (q, J=7.2 Hz, 2H), 1.44 (t, J=7.3 Hz, 3H).

Step 2: Synthesis of 1-(difluoromethyl)-3-phenyl-4-(trifluoromethyl)-1H-pyrazole-5-carboxylic acid (INT 32): 1.2 M aqueous LiOH (114 μL, 0.136 mmol, 1.30 eq) was added to a solution of ethyl 1-(difluoromethyl)-3-phenyl-4-(trifluoromethyl)-1H-pyrazole-5-carboxylate (35 mg, 0.105 mmol, 1.00 eq) in MeOH (1 mL) was added and the reaction was stirred at 40° C. for 2.5 hours. The solvent was removed under reduced pressure to give the title compound, which was used in the next step without further purification.

Step 3: Synthesis of N-(5-chloro-6-(2H-1,2,3-triazol-2-yl) pyridin-3-yl)-1-(difluoromethyl)-3-phenyl-4-(trifluoromethyl)-1H-pyrazole-5-carboxamide (1-179): POCl₃ (19 μL, 0.204 mmol, 1.95 eq) was added to a solution of 1-(difluoromethyl)-3-phenyl-4-(trifluoromethyl)-1H-pyrazole-5-carboxylic acid (32 mg, 0.105 mmol, 1.00 eq) and 5-chloro-6-(2H-1,2,3-triazol-2-yl) pyridin-3-amine (24 mg, 0.123 mmol, 1.17 eq) in pyridine (1 mL) and the reaction was stirred at ambient temperature for 4 hours. The reaction was quenched with 1 M aqueous K₂CO₃ and the mixture was extracted with EtOAc. The combined organic extracts were dried (MgSO₄) and evaporated and the residue was successively purified by column chromatography eluting with 5-50% EtOAc in cyclohexane and by reverse phase HPLC to give the title compound (2.71 mg, 0.006 mmol, 5%). (ES, m/z): [M+H]⁺484.2, ¹H NMR (400 MHz, DMSO) δ 12.23 (s, 1H), 8.70 (s, 1H), 8.59 (d, J=2.0 Hz, 1H), 8.19 (t, J=56.8 Hz, 1H), 8.19 (s, 2H), 7.65-7.62 (m, 2H), 7.59-7.55 (m, 3H).

### Example 20—Synthesis of Additional Compounds

The compound in Table 12 was synthesized from ethyl 1-(difluoromethyl)-5-phenyl-4-(trifluoromethyl)-1H-pyrazole-3-carboxylate using the route described above.

TABLE 12

| Compound | | Starting | |
|---|---|---|---|
| No. | Structure | Material | Analytical data |
| I-180 | 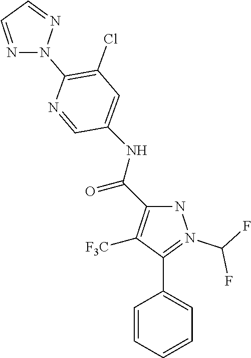 | 5-chloro-6-(2H- 1,2,3-triazol-2- yl)pyridin-3- amine | (ES, m/z): [M + H]⁺ 484.2, ¹H NMR (400 MHz, DMSO) δ 11.53 (s, 1H), 8.98 (d, J = 2.3 Hz, 1H), 8.72 (d, J = 2.3 Hz, 1H), 8.20 (s, 2H), 7.84-7.55 (m, 6H) |
| I-181 | 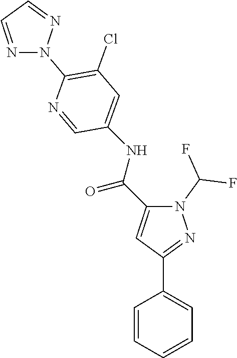 [SMILES: O=C(Nc1cnc(-n2nccn2)c(Cl)c1)c1cc(-c2ccccc2)nn1C(F)F] | 1- (difluoromethyl)- 3-phenyl-1H- pyrazole-5- carboxylic acid | (ES, m/z): [M + H]⁺ 416.1 ¹H NMR (400 MHz, DMSO) δ 11.40 (s, 1H), 8.92 (d, J = 2.2 Hz, 1H), 8.73 (d, J = 2.3 Hz, 1H), 8.36 (t, J = 58.3 Hz, 1H), 8.25 (s, 2H), 8.00-7.96 (m, 2H), 7.81 (s, 1H), 7.62-7.52 (m, 3H). |
| I-182 | 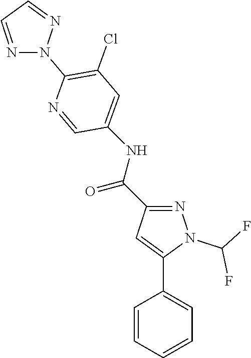 [SMILES: O=C(Nc1cnc(-n2nccn2)c(Cl)c1)c1cc(-c2ccccc2)n(C(F)F)n1] | 5-chloro-6-(2H- 1,2,3-triazol-2- yl)pyridin-3- amine | (ES, m/z): [M + H]⁺ 416.2, ¹H NMR (400 MHz, CDCl₃) δ 9.03-9.00 (m, 1H), 8.79 (d, J = 2.4 Hz, 1H), 8.68- 8.67 (m, 1H), 7.95-7.94 (m, 2H), 7.56-7.53 (m, 3H), 7.52-7.48 (m, 2H), 7.26 (s, 1H), 7.19 (t, J = 58.0 Hz, 1H), 7.06 (s, 1H) |

### Example 21—Synthesis of 2-phenyl-3-(trifluoromethyl)-5-(2-(trifluoromethyl)pyridin-4-yl)-5,6,7,8-tetrahydro-4H-pyrazolo[1,5-a][1,4]diazepin-4 one (I-183)

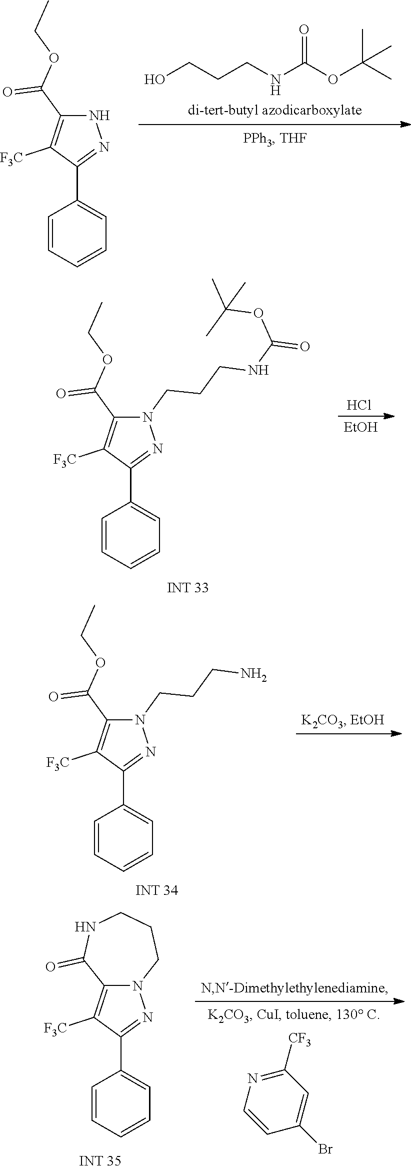

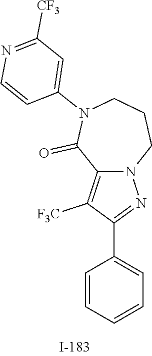 [SMILES: O=C1c2c(C(F)(F)F)c(-c3ccccc3)nn2CCCN1c1ccnc(C(F)(F)F)c1]

Step 1: Synthesis of ethyl 1-(3-((tert-butoxycarbonyl)amino) propyl)-3-phenyl-4-(trifluoromethyl)-1H-pyrazole-5-carboxylate (INT 33): di-tert-Butyl azodicarboxylate (262 mg, 1.14 mmol, 1.23 eq) was added in portions to an ice-cooled solution of ethyl 3-phenyl-4-(trifluoromethyl)-1H-pyrazole-5-carboxylate (262 mg, 0.922 mmol, 1.00 eq), PPh₃ (303 mg, 1.16 mmol, 1.25 eq) and tert-butyl (3-hydroxypropyl) carbamate (195 μL, 1.14 mmol, 1.24 eq) in THF (5 mL) and the reaction was stirred at rt for 16 hours. The solvent was removed under reduced pressure, and the crude residue was purified by column chromatography eluting with 5-50% EtOAc in cyclohexane to give the title compound as a clear oil (490 mg, 1.11 mmol, quant.). ¹H NMR (400 MHz, CDCl₃) δ 7.56-7.51 (m, 2H), 7.44-7.41 (m, 3H), 4.92 (s, 1H), 4.52 (t, J=6.7 Hz, 2H), 4.45 (dd, J=6.9, 14.1 Hz, 2H), 3.22-3.16 (m, 2H), 2.12 (tt, J=6.5, 6.6 Hz, 2H), 1.45-1.41 (m, 12H).

Step 2: Synthesis of ethyl 1-(3-aminopropyl)-3-phenyl-4-(trifluoromethyl)-1H-pyrazole-5-carboxylate (INT 34): 4 M HCl solution in 1,4-dioxane (1.7 mL, 6.66 mmol, 6.00 eq) was added to a stirred solution of ethyl 1-(3-((tert-butoxycarbonyl)amino) propyl)-3-phenyl-4-(trifluoromethyl)-1H-pyrazole-5-carboxylate (490 mg, 1.11 mmol, 1.00 eq) in EtOH (15 mL) and the reaction was stirred at rt for 3 hours. More 4 M HCl solution in 1,4-dioxane (2.0 mL, 8.00 mmol, 7.21 eq) was added and the reaction was stirred at 50° C. for 30 minutes. The reaction was cooled to rt and concentrated to give the title compound, which was used in the next step without further purification.

Step 3: Synthesis of 2-phenyl-3-(trifluoromethyl)-5,6,7,8-tetrahydro-4H-pyrazolo[1,5-a][1,4]diazepin-4-one (INT 35): K₂CO₃ (890 mg, 6.44 mmol, 5.81 eq) was added to a stirred suspension of ethyl 1-(3-aminopropyl)-3-phenyl-4-(trifluoromethyl)-1H-pyrazole-5-carboxylate (378 mg, 1.11 mmol, 1.00 eq) in EtOH (15 mL) and the reaction was stirred at rt for 72 hours. The solvent was removed under reduced pressure and the residue was suspended in water (3 mL) and filtered. The solid was washed with water and dried to give the title compound as a white solid (184 mg, 0.59 mmol, 53.5%). (ES, m/z): [M+H]⁺ 296.2, ¹H NMR (400 MHz, DMSO) δ 8.73 (t, J=5.6 Hz, 1H), 7.57-7.45 (m, 5H), 4.49 (t, J=6.9 Hz, 2H), 3.20-3.13 (m, 2H), 2.16 (tt, J=6.8, 6.8 Hz, 2H).

Step 4: Synthesis of 2-phenyl-3-(trifluoromethyl)-5-(2-(trifluoromethyl)pyridin-4-yl)-5,6,7,8-tetrahydro-4H-pyrazolo[1,5-a][1,4]diazepin-4-one (I-183): N,N-Dimethylethylenediamine (0.067 mL, 0.624 mmol, 6.14 eq) and CuI (41 mg, 0.215 mmol, 2.12 eq) were added to a stirred degassed mixture of 4-bromo-2-(trifluoromethyl)pyridine (51 mg, 0.226 mmol, 2.22 eq), 2-phenyl-3-(trifluoromethyl)-5,6,7,8-tetrahydro-4H-pyrazolo[1,5-a][1,4]diazepin-4-one (30 mg, 0.102 mmol, 1.00 eq) and K₂CO₃ (31 mg, 0.224 mmol, 2.21 eq) in toluene (1 mL). The reaction mixture was stirred at 130° C. in the microwave for 5.5 hours. The reaction was cooled to rt, diluted with EtOAc and filtered through Celite®. The solvent was evaporated under reduced pressure and the crude residue was purified by column chromatography eluting with 20-100% EtOAc in cyclohexane to give the title compound as an off-white solid (24.1 mg, 0.54 mmol, 53%). (ES, m/z): [M+H]⁺ 441.3, ¹H NMR (400 MHz, DMSO) δ 8.85 (d, J=5.5 Hz, 1H), 8.30 (d, J=1.9 Hz, 1H), 7.97 (dd, J=2.1, 5.5 Hz, 1H), 7.59 (dd, J=1.9, 7.7 Hz, 2H), 7.56-7.48 (m, 3H), 4.72 (t, J=6.8 Hz, 2H), 4.04 (t, J=6.3 Hz, 2H), 2.37 (tt, J=6.6, 6.6 Hz, 2H).

### Example 22—Synthesis of Additional Compounds

Table 13 provides exemplary compounds synthesized from ethyl 3-phenyl-4-(trifluoromethyl)-1H-pyrazole-5-carboxylate using the route described above.

TABLE 13

| Compound | | Coupling | |
|---|---|---|---|
| No. | Structure | reagents | NMR |
| I-184 | 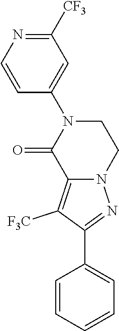 [SMILES: O=C1c2c(C(F)(F)F)c(-c3ccccc3)nn2CCN1c1ccnc(C(F)(F)F)c1] | tert-butyl (2- hydroxyethyl)car- bamate and 4- bromo-2- (trifluorometh- yl)pyridine | (ES, m/z): [M + H]⁺ 427.5, ¹H NMR (400 MHz, DMSO) δ 8.84 (d, J = 5.5 Hz, 1H), 8.06 (d, J = 1.9 Hz, 1H), 7.83 (dd, J = 1.9, 5.5 Hz, 1H), 7.52- 7.50 (m, 5H), 4.72-4.67 (m, 2H), 4.50-4.45 (m, 2H) |
| I-185 | 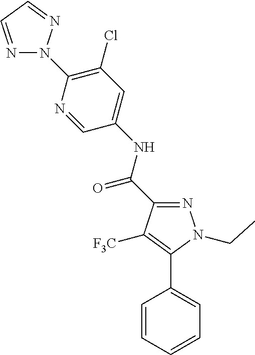 | tert-butyl (2- hydroxyethyl)car- bamate and 5- bromo-2-(2H- 1,2,3-triazol-2- yl)pyridine | (ES, m/z): [M + H]⁺ 426.5, ¹H NMR (400 MHz, DMSO) δ 8.71 (d, J = 2.3 Hz, 1H), 8.22 (s, 2H), 8.19 (dd, J = 2.6, 8.7 Hz, 1H), 8.14 (d, J = 8.7 Hz, 1H), 7.52-7.50 (m, 5H), 4.76-4.70 (m, 2H), 4.45- 4.40 (m, 2H) |

### Example 23—Synthesis of 3-(benzofuran-7-yl)-N-(5-cyano-6-(2H-1,2,3-triazol-2-yl) pyridin-3-yl)-1-(2-(dimethylamino)ethyl)-4-(trifluoromethyl)-1H-pyrazole-5-carboxamide (I-186)

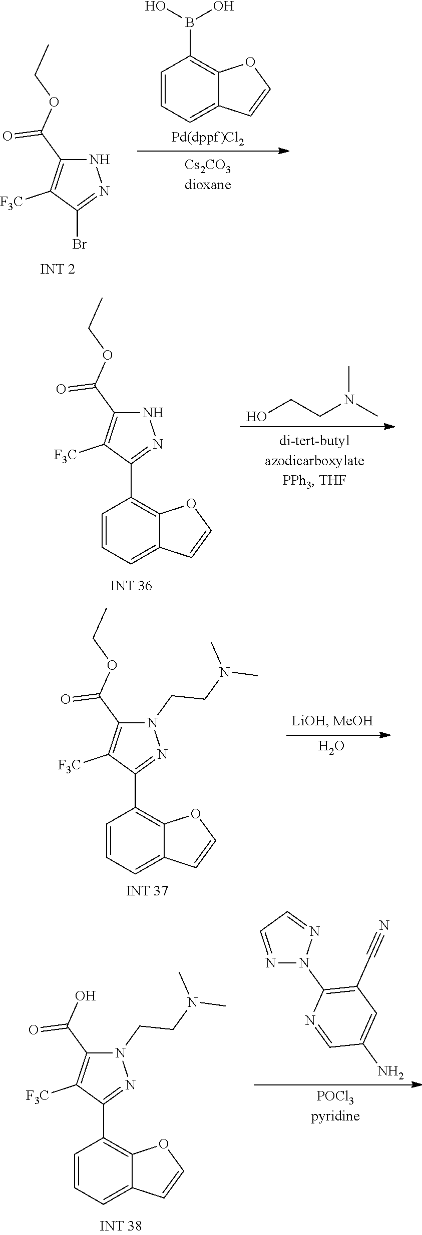

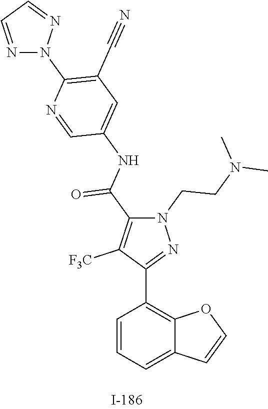 [SMILES: CN(C)CCn1nc(-c2cccc3ccoc23)c(C(F)(F)F)c1C(=O)Nc1cnc(-n2nccn2)c(C#N)c1]

Step 1: Synthesis of ethyl 3-(benzofuran-7-yl)-4-(trifluoromethyl)-1H-pyrazole-5-carboxylate (INT 36): Pd(dppf)Cl₂ (50 mg, 0.0683 mmol, 0.125 eq) was added to a stirred degassed suspension of benzofuran-7-ylboronic acid (100 mg, 0.617 mmol, 1.13 eq), Cs₂CO₃ (442 mg, 1.36 mmol, 2.48 eq) and ethyl 3-bromo-4-(trifluoromethyl)-1H-pyrazole-5-carboxylate (157 mg, 0.547 mmol, 1.00 eq) in 1,4-dioxane (3 mL) and water (0.50 mL) and the reaction was stirred at 95° C. for 6.5 hours. The solvent was removed, and the crude material partitioned between EtOAc and water. The organic layer was separated, dried (MgSO₄) and evaporated. The crude residue was purified by column chromatography eluting with 5-100% EtOAc in cyclohexane to give the title compound as a clear oil (105 mg, 0.295 mmol, 53%). ¹H NMR (400 MHz, CDCl₃) δ 11.54 (s, 1H), 7.73-7.70 (m, 1H), 7.65-7.64 (m, 1H), 7.42 (d, J=7.4 Hz, 1H), 7.33 (t, J=7.3 Hz, 1H), 6.86-6.84 (m, 1H), 4.41 (q, J=7.2 Hz, 2H), 1.39 (t, J=7.2 Hz, 3H).

Step 2: Synthesis of ethyl 3-(benzofuran-7-yl)-1-(2-(dimethylamino)ethyl)-4-(trifluoromethyl)-1H-pyrazole-5-carboxylate (INT 37): di-tert-Butyl azodicarboxylate (91 mg, 0.395 mmol, 1.22 eq) was added in portions to an ice-cooled solution of ethyl 3-(benzofuran-7-yl)-4-(trifluoromethyl)-1H-pyrazole-5-carboxylate (105 mg, 0.324 mmol, 1.00 eq), PPh₃ (105 mg, 0.400 mmol, 1.24 eq) and 2-dimethylaminoethanol (40 μL, 0.393 mmol, 1.21 eq) in THF (2 mL) and the reaction was stirred at rt for 3.5 hours. The solvent was removed under reduced pressure, and the crude residue was successively purified by column chromatography eluting with 10-100% EtOAc in cyclohexane and by SCX, eluting with 3.5 N NH₃ in MeOH, to give the title compound as a clear oil (98 mg, 0.248 mmol, 76%). ¹H NMR (400 MHz, CDCl₃) δ 7.66 (dd, J=1.2, 7.8 Hz, 1H), 7.64 (d, J=2.1 Hz, 1H), 7.39-7.26 (m, 2H), 6.81 (d, J=2.2 Hz, 1H), 4.68-4.64 (m, 2H), 4.49-4.42 (m, 2H), 2.78 (t, J=6.7 Hz, 2H), 2.29 (s, 6H), 1.43 (t, J=7.2 Hz, 3H).

Step 3: Synthesis of 3-(benzofuran-7-yl)-1-(2-(dimethylamino)ethyl)-4-(trifluoromethyl)-1H-pyrazole-5-carboxylic acid (INT 38): 1 M aqueous LiOH (296 μL, 0.296 mmol, 1.19 eq) was added to a solution of ethyl 3-(benzofuran-7-yl)-1-(2-(dimethylamino)ethyl)-4-(trifluoromethyl)-1H-pyrazole-5-carboxylate (98 mg, 0.248 mmol, 1 eq) in MeOH (2 mL) and the reaction was stirred at 40° C. for 3.5 hours. The solvents were evaporated under reduced pressure and the residue was directly used in the next step used without further purification.

Step 4: Synthesis of 3-(benzofuran-7-yl)-N-(5-cyano-6-(2H-1,2,3-triazol-2-yl) pyridin-3-yl)-1-(2-(dimethylamino)ethyl)-4-(trifluoromethyl)-1H-pyrazole-5-carboxamide (I-186): POCl₃ (39 μL, 0.423 mmol, 1.71 eq) was added to a solution 3-(benzofuran-7-yl)-1-(2-(dimethylamino)ethyl)-4-(trifluoromethyl)-1H-pyrazole-5-carboxylic acid (91 mg, 0.248 mmol, 1.00 eq) and 5-amino-2-(triazol-2-yl)pyridine-3-carbonitrile (53 mg, 0.285 mmol, 1.15 eq) in pyridine (2.50 mL) and the reaction was stirred at rt for 30 minutes. The reaction mixture was partitioned between EtOAc and 1 M aqueous K₂CO₃ and the aqueous phase was separated and extracted with EtOAc. The combined organic phases were dried (MgSO₄) and evaporated. The crude residue was purified by column chromatography eluting with 20-100% EtOAc in cyclohexane followed by 0-100% 3:1 EtOAc EtOH in cyclohexane to give an orange oil that was recrystallized from EtOH to give the title compound (22 mg, 0.04 mmol, 16%) as a white solid. (ES, m/z): [M+H]⁺ 536.4, ¹H NMR (400 MHz, DMSO) δ 9.08 (d, J=2.2 Hz, 1H), 8.85 (d, J=2.4 Hz, 1H), 8.33 (s, 2H), 8.07 (d, J=2.1 Hz, 1H), 7.83-7.79 (m, 1H), 7.41-7.38 (m, 2H), 7.08 (d, J=2.1 Hz, 1H), 4.46 (t, J=6.0 Hz, 2H), 2.75 (t, J=5.7 Hz, 2H), 2.14 (s, 6H).

### Example 24—Synthesis of 1-methyl-3-(4-oxo-3,4-dihydroquinazolin-8-yl)-4-(trifluoromethyl)-N-(2-(trifluoromethyl)pyridin-4-yl)-1H-pyrazole-5-carboxamide (I-187)

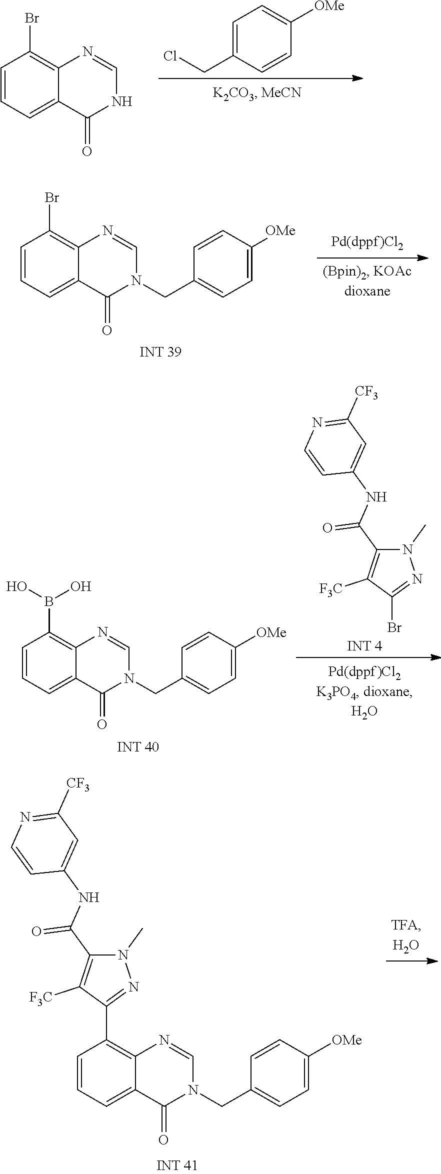 [SMILES: O=c1[nH]cnc2c(Br)cccc12 ; COc1ccc(CCl)cc1 ; COc1ccc(Cn2cnc3c(Br)cccc3c2=O)cc1 ; COc1ccc(Cn2cnc3c(B(O)O)cccc3c2=O)cc1 ; Cn1nc(Br)c(C(F)(F)F)c1C(=O)Nc1ccnc(C(F)(F)F)c1 ; COc1ccc(Cn2cnc3c(-c4nn(C)c(C(=O)Nc5ccnc(C(F)(F)F)c5)c4C(F)(F)F)cccc3c2=O)cc1]

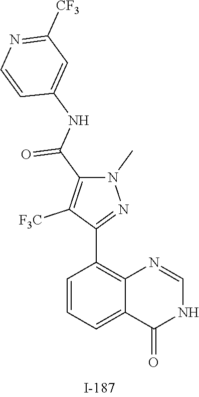 [SMILES: Cn1nc(-c2cccc3c(=O)[nH]cnc23)c(C(F)(F)F)c1C(=O)Nc1ccnc(C(F)(F)F)c1]

Step 1: Synthesis of 8-bromo-3-(4-methoxybenzyl) quinazolin-4 (3H)-one (INT 39): K₂CO₃ (737 mg, 5.33 mmol, 3.00 eq) was added to a stirred solution of 8-bromoquinazolin-4 (3H)-one (400 mg, 1.78 mmol, 1.00 eq), 4-methoxybenzyl chloride (0.36 mL, 2.67 mmol, 1.50 eq) in MeCN (9 mL) was stirred at 65° C. for 16 hours. The reaction mixture was sonicated, diluted with additional MeCN (5 mL) and stirred at 65° C. for 16 hours. The reaction was partitioned between water and EtOAc. The organic layer was separated, washed with brine, dried (MgSO₄), filtered, and concentrated under reduced pressure. The crude residue was purified by column chromatography eluting with 0-100% EtOAc in cyclohexane to give the title compound as a white solid (340 mg, 0.98 mmol, 55%). ¹H NMR (400 MHz, CDCl₃) δ 8.29 (dd, J=1.4, 8.0 Hz, 1H), 8.22 (s, 1H), 8.03 (dd, J=1.5, 7.8 Hz, 1H), 7.36 (dd, J=7.9, 7.9 Hz, 1H), 7.33-7.29 (m, 2H), 6.89-6.86 (m, 2H), 5.13 (s, 2H), 3.79 (s, 3H).

Step 2: Synthesis of (3-(4-methoxybenzyl)-4-oxo-3,4-dihydroquinazolin-8-yl) boronic acid (INT 40): Pd(dppf)Cl₂ (81 mg, 0.10 mmol, 0.1000 eq) was added to a degassed solution of 8-bromo-3-(4-methoxybenzyl) quinazolin-4 (3H)-one (340 mg, 0.10 mmol, 1.00 eq), bis(pinacolato)diboron (625 mg, 2.46 mmol, 2.50 eq), and KOAc (290 mg, 2.95 mmol, 3.00 eq) in 1,4-dioxane (7 mL) and the reaction was stirred at 100° C. for 90 minutes. The reaction mixture was partitioned between EtOAc and water. The organic phase was separated, washed with brine, dried (MgSO₄), filtered, and concentrated under reduced pressure to give the title compound as a brown solid (380 mg, 1.23 mmol, quant.), which was used in the next step without further purification. (ES, m/z): [M+H]⁺ 311.

Step 3: Synthesis of 3-(3-(4-methoxybenzyl)-4-oxo-3,4-dihydroquinazolin-8-yl)-1-methyl-4-(trifluoromethyl)-N-(2-(trifluoromethyl)pyridin-4-yl)-1H-pyrazole-5-carboxamide (INT 41): Pd(dppf)Cl₂ (20 mg, 0.0240 mmol, 0.10 eq) was added to a stirred degassed solution of (3-(4-methoxybenzyl)-4-oxo-3,4-dihydroquinazolin-8-yl) boronic acid (380 mg, 1.23 mmol, 5.11 eq), 3-bromo-1-methyl-4-(trifluoromethyl)-N-(2-(trifluoromethyl)pyridin-4-yl)-1H-pyrazole-5-carboxamide (100 mg, 0.240 mmol, 1.00 eq), and K₃PO₄ (166 mg, 0.719 mmol, 3.00 eq) in 1,4-dioxane (2 mL) and water (0.3 mL) at 50° C. The reaction mixture was then stirred at 100° C. for 2 hours. Once cooled to rt before, the crude mixture was filtered through Celite®, which was washed with EtOAc. The filtrate was concentrated under reduced pressure, redissolved in EtOAc and washed with saturated aqueous NaHCO₃ and brine. The organic phase was dried (MgSO₄), filtered, and concentrated under reduced pressure. The crude residue was purified by column chromatography eluting with 0-100% EtOAc in cyclohexane to give the title compound as a brown oil (150 mg, 0.249 mmol, quant.). (ES, m/z): [M−H]⁻ 603.

Step 4: Synthesis of 1-methyl-3-(4-oxo-3,4-dihydroquinazolin-8-yl)-4-(trifluoromethyl)-N-(2-(trifluoromethyl)pyridin-4-yl)-1H-pyrazole-5-carboxamide (I-187): 3-(3-(4-Methoxybenzyl)-4-oxo-3,4-dihydroquinazolin-8-yl)-1-methyl-4-(trifluoromethyl)-N-(2-(trifluoromethyl)pyridin-4-yl)-1H-pyrazole-5-carboxamide (110 mg, 0.183 mmol, 1.00 eq) was dissolved in TFA (2.0 mL, 26.1 mmol, 143 eq) and water (0.050 mL, 2.77 mmol, 15.2 eq) and the reaction was stirred at 100° C. in a sealed tube for 16 hours. The mixture was allowed to cool to rt and concentrated under reduced pressure. The crude residue was partitioned between EtOAc and saturated aqueous NaHCO₃. The organic phase was separated, washed with brine, dried (MgSO₄), filtered, and concentrated under reduced pressure. The crude residue was successively purified by column chromatography eluting with 0-100% EtOAc in cyclohexane and by reverse phase HPLC to give the title compound as an off-white solid (20 mg, 0.0419 mmol, 23%). (ES, m/z): [M+H]⁺ 483, ¹H NMR (400 MHz, DMSO) δ 12.42 (s, 1H), 11.98 (s, 1H), 8.74 (s, 1H), 8.27 (dd, J=1.5, 8.0 Hz, 1H), 8.21 (s, 1H), 8.07 (s, 1H), 7.91 (s, 1H), 7.81 (dd, J=1.6, 7.1 Hz, 1H), 7.62 (dd, J=7.6, 7.6 Hz, 1H), 4.02 (s, 3H).

### Example 25—Synthesis of N-(5-chloro-6-(2H-1,2,3-triazol-2-yl) pyridin-3-yl)-1-(trifluoromethyl)-5,6-dihydropyrazolo[5,1-a]isoquinoline-2-carboxamide (I-188)

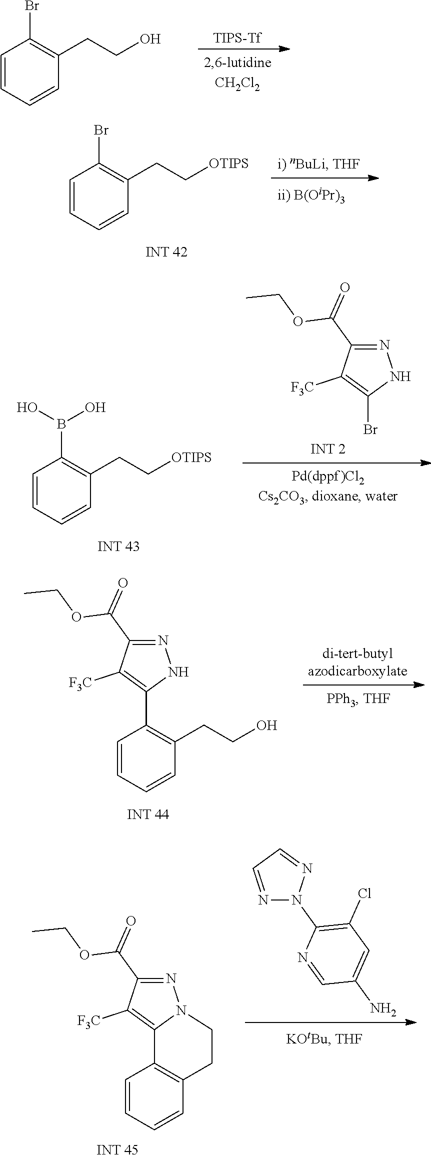

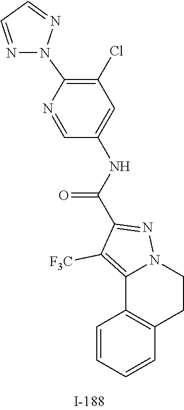 [SMILES: O=C(Nc1cnc(-n2nccn2)c(Cl)c1)c1nn2c(c1C(F)(F)F)-c1ccccc1CC2]

Step 1: Synthesis of (2-bromophenethoxy)triisopropylsilane (INT 42): 2,6-Lutidine (1.7 mL, 14.9 mmol, 3.00 eq) was added to an ice-cooled stirred solution of 2-(2-bromophenyl) ethan-1-ol (0.67 mL, 4.97 mmol, 1.00 eq) in DCM (25 mL) and the reaction was stirred for 5 minutes. Triisopropylsilyl trifluoromethanesulfonate (2.7 mL, 9.95 mmol, 2.00 eq) was added and the reaction was allowed to reach rt and stirred for 2 hours. The mixture was quenched with saturated aqueous NaHCO₃ and extracted with DCM. The organic phase was separated, dried (MgSO₄), filtered, and concentrated. The crude residue was purified by column chromatography eluting with 0-50% EtOAc in cyclohexane to give the title compound as a clear oil (964 mg, 2.70 mmol, 54%). ¹H NMR (400 MHz, DMSO) δ 7.58 (dd, J=1.1, 8.1 Hz, 1H), 7.37 (dd, J=1.8, 7.5 Hz, 1H), 7.31 (ddd, J=7.4, 7.4, 1.2 Hz, 1H), 7.16 (ddd, J=7.6, 7.6, 1.8 Hz, 1H), 3.87 (t, J=6.8 Hz, 2H), 2.94 (t, J=6.8 Hz, 2H), 1.05-0.96 (m, 21H).

Step 2: Synthesis of (2-(2-((triisopropylsilyl)oxy)ethyl)phenyl) boronic acid (INT 43): 2.5 M n-BuLi in hexanes (1.5 mL, 3.86 mmol, 1.50 eq) was added dropwise to a solution of (2-bromophenethoxy)triisopropylsilane (920 mg, 2.57 mmol, 1.00 eq) in THF (25 mL) at −78° C. and the reaction was stirred for 45 minutes. Triisopropyl borate (1.5 mL, 6.44 mmol, 2.50 eq) was added and the reaction was allowed to warm to rt and stirred for 2 hours. The reaction mixture was cooled to 0° C. and quenched with 2 M aqueous HCl. The reaction mixture was washed with dilute HCl and extracted with Et₂O. The organic phase was washed with brine, dried (MgSO₄), filtered and concentrated under reduced pressure to give the title compound as a clear oil (630 mg, 1.95 mmol, 76%) that was used in the next step without further purification. (ES, m/z): [M+H]⁺ 323.

Step 3: Synthesis of ethyl 5-(2-(2-hydroxyethyl)phenyl)-4-(trifluoromethyl)-1H-pyrazole-3-carboxylate (INT 44): Pd(dppf)Cl₂ (86 mg, 0.105 mmol, 0.100 eq) was added to a degassed stirred solution of ethyl 5-bromo-4-(trifluoromethyl)-1H-pyrazole-3-carboxylate (300 mg, 1.05 mmol, 1.00 eq), (2-(2-((triisopropylsilyl)oxy)ethyl)phenyl) boronic acid (650 mg, 2.02 mmol, 1.93 eq) and Cs₂CO₃ (1,022 mg, 3.14 mmol, 3.00 eq) in 1,4-dioxane (10 mL) and water (1 mL) and the reaction mixture stirred at 90° C. for 16 hours. After cooling to rt, the crude reaction was filtered through Celite®, which was washed with EtOAc. The filtrate was concentrated under reduced pressure and the residue was partitioned between water and EtOAc. The aqueous phase was separated and extracted with EtOAc. The combined organic phases were washed with brine, dried (MgSO₄), filtered, and concentrated under reduced pressure. The crude residue was purified by column chromatography eluting with 0-80% EtOAc in cyclohexane to give the title compound as an off-white solid (205 mg, 0.624 mmol, 60%). (ES, m/z): [M+H]⁺ 329.

Step 4: Synthesis of ethyl 1-(trifluoromethyl)-5,6-dihydropyrazolo[5,1-a]isoquinoline-2-carboxylate (INT 45): di-tert-Butyl azodicarboxylate (168 mg, 0.731 mmol, 1.20 eq) was added to a cooled solution of ethyl 5-(2-(2-hydroxyethyl)phenyl)-4-(trifluoromethyl)-1H-pyrazole-3-carboxylate (200 mg, 0.609 mmol, 1.00 eq) and PPh₃ (192 mg, 0.731 mmol, 1.20 eq) in THF (7 mL). The reaction was warmed to rt and stirred for 16 hours. The solvent was removed under reduced pressure and the crude residue was purified by column chromatography eluting with 0-100% EtOAc in cyclohexane to give a brown solid, which was triturated in cyclohexane. The filtrate was concentrated under reduced pressure to give the title compound as an off-white solid (110 mg, 0.355 mmol, 58%). ¹H NMR (400 MHz, CDCl₃) δ 7.78-7.76 (m, 1H), 7.39 (dd, J=3.3, 5.7 Hz, 2H), 7.36-7.30 (m, 1H), 4.46 (q, J=7.2 Hz, 2H), 4.38 (t, J=6.8 Hz, 2H), 3.18 (t, J=6.7 Hz, 2H), 1.43 (t, J=7.2 Hz, 3H).

Step 5: Synthesis of N-(5-chloro-6-(2H-1,2,3-triazol-2-yl) pyridin-3-yl)-1-(trifluoromethyl)-5,6-dihydropyrazolo[5,1-a]isoquinoline-2-carboxamide (I-188): The title compound was synthesized from INT 48 based on procedures described in Example 6 and isolated by reverse phase HPLC as an an orange solid (17 mg, 0.0363 mmol, 11%). (ES, m/z): [M+H]⁺ 460.0, ¹H NMR (400 MHz, DMSO) δ d 11.41 (s, 1H), 8.93 (d, J=2.3 Hz, 1H), 8.69 (d, J=2.3 Hz, 1H), 8.18 (s, 2H), 7.71-7.66 (m, 1H), 7.54-7.48 (m, 3H), 4.46 (t, J=6.8 Hz, 2H), 3.26 (t, J=6.8 Hz, 2H).

### Example 26—Synthesis of N-(5-chloro-6-(2H-1,2,3-triazol-2-yl) pyridin-3-yl)-1-cyclopropyl-3-phenyl-4-(trifluoromethyl)-1H-pyrazole-5-carboxamide (I-189)

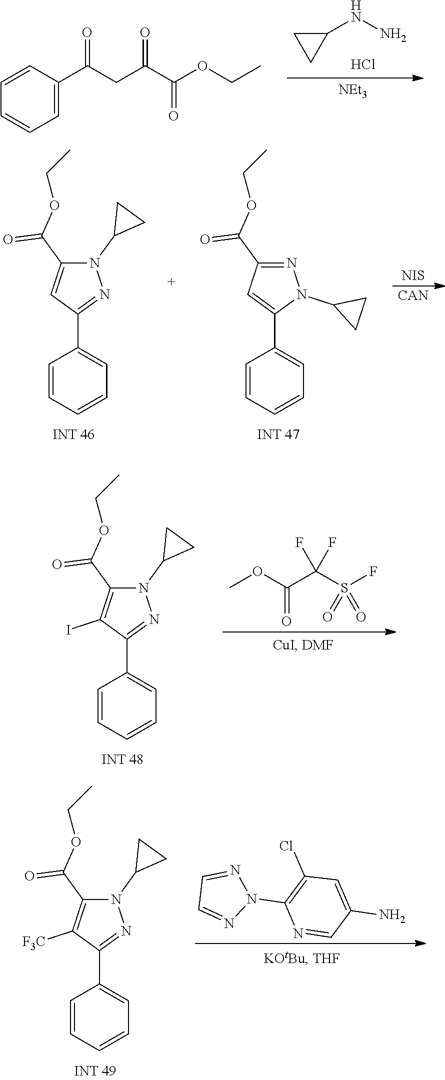

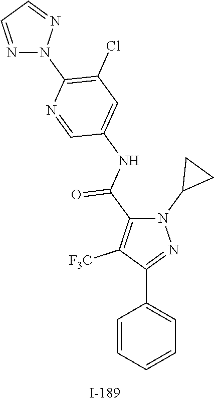 [SMILES: O=C(Nc1cnc(-n2nccn2)c(Cl)c1)c1c(C(F)(F)F)c(-c2ccccc2)nn1C1CC1]

Step 1: Synthesis of ethyl 1-cyclopropyl-3-phenyl-1H-pyrazole-5-carboxylate (INT 46): NEt₃ (1.4 mL, 10.3 mmol, 1.12 eq) was added to a stirred solution of cyclopropylhydrazine hydrochloride (993 mg, 9.15 mmol, 1.00 eq) and ethyl 4-phenyl-2,4-dioxobutanoate (2.25 g, 10.2 mmol, 1.12 eq) in EtOH (60 mL) was refluxed for 3 hours. The solvent was removed under reduced pressure and the residue was partitioned between water and EtOAc. The aqueous layer was separated and extracted with EtOAc. The combined organic layers were dried (MgSO₄) and evaporated. The crude residue was purified by column chromatography eluting with 5-50% EtOAc in cyclohexane to give the title compound as a clear oil (824 mg, 3.2 mmol, 35%). ¹H NMR (400 MHz, CDCl₃) δ 7.78 (d, J=7.3 Hz, 2H), 7.38 (t, J=7.5 Hz, 2H), 7.32-7.27 (m, 1H), 7.12 (s, 1H), 4.39 (q, J=7.1 Hz, 2H), 4.38-4.31 (m, 1H), 1.41 (t, J=7.1 Hz, 3H), 1.39-1.33 (m, 2H), 1.10-1.04 (m, 2H). Regioisomeric ethyl 1-cyclopropyl-5-phenyl-4-(trifluoromethyl)-1H-pyrazole-3-carboxylate (INT 47) was also isolated by column chromatography as a clear oil (570 mg, 2.2 mmol, 24%). ¹H NMR (400 MHz, CDCl₃) δ 7.58-7.55 (m, 2H), 7.49-7.40 (m, 3H), 6.85 (s, 1H), 4.41 (q, J=7.1 Hz, 2H), 3.67-3.60 (m, 1H), 1.40 (t, J=7.1 Hz, 3H), 1.26-1.21 (m, 2H), 1.02-0.96 (m, 2H).

Step 2: Synthesis of ethyl 1-cyclopropyl-4-iodo-3-phenyl-1H-pyrazole-5-carboxylate (INT 48): Ammonium cerium (IV) nitrate (199 mg, 0.363 mmol, 0.100 eq) was added to a suspension of ethyl 1-cyclopropyl-3-phenyl-1H-pyrazole-5-carboxylate (930 mg, 3.63 mmol, 1.00 eq), NIS (1143 mg, 5.08 mmol, 1.40 eq) in MeCN (5.0 mL), the reaction was stirred at 70° C. for 16 hours. The solvent was removed under reduced pressure and the crude residue was purified by column chromatography eluting with 0-80% EtOAc in cyclohexane to give the title compound as a brown oil (644 mg, 1.69 mmol, 46%). ¹H NMR (400 MHz, DMSO) δ 7.71-7.68 (m, 2H), 7.50-7.42 (m, 3H), 4.41 (q, J=7.1 Hz, 2H), 4.21-4.14 (m, 1H), 1.40 (t, J=7.1 Hz, 3H), 1.20-1.15 (m, 2H), 1.08-1.02 (m, 2H).

Step 3: Synthesis of ethyl 1-cyclopropyl-3-phenyl-4-(trifluoromethyl)-1H-pyrazole-5-carboxylate (INT 49): CuI (638 mg, 3.35 mmol, 2.00 eq) and methyl 2,2-difluoro-2-(fluorosulfonyl)acetate (0.43 mL, 3.35 mmol, 2.00 eq) were added to a solution of ethyl 1-cyclopropyl-4-iodo-3-phenyl-1H-pyrazole-5-carboxylate (640 mg, 1.67 mmol, 1.00 eq) in DMF (9 mL) and the reaction was stirred at 90° C. for 4 hours. Once cooled to rt, the reaction was filtered through Celite®, which was washed with EtOAc. The filtrate was washed with water, brine, dried (MgSO₄), filtered and evaporated. The crude residue was purified by column chromatography eluting with 0-80% EtOAc in cyclohexane to give the title compound as a clear oil (364 mg, 1.12 mmol, 67%). ¹H NMR (400 MHz, DMSO) δ 7.50-7.46 (m, 5H), 4.45 (q, J=7.1 Hz, 2H), 4.09-4.03 (m, 1H), 1.35 (t, J=7.3 Hz, 3H), 1.20-1.17 (m, 2H), 1.11-1.05 (m, 2H).

Step 4: Synthesis of N-(5-chloro-6-(2H-1,2,3-triazol-2-yl) pyridin-3-yl)-1-cyclopropyl-3-phenyl-4-(trifluoromethyl)-1H-pyrazole-5-carboxamide (I-189): The title compound was synthesized from INT 49 based on procedures described in Example 6 and isolated by reverse phase HPLC as an off-white solid (41 mg, 0.0843 mmol, 34%). (ES, m/z): [M+H]⁺ 474.0, ¹H NMR (400 MHz, DMSO) δ 11.99 (s, 1H), 8.81 (d, J=2.3 Hz, 1H), 8.62 (d, J=2.3 Hz, 1H), 8.20 (s, 2H), 7.60 (dd, J=1.8, 7.6 Hz, 2H), 7.54-7.50 (m, 3H), 3.93-3.86 (m, 1H), 1.23-1.18 (m, 2H), 1.11-1.05 (m, 2H).

### Example 27—Synthesis of Additional Compounds

Exemplary compounds synthesized using the route described above are provided in Table 14.

TABLE 14

| Compound | | Coupling | |
|---|---|---|---|
| No. | Structure | partner | Analytical data |
| I-190 | 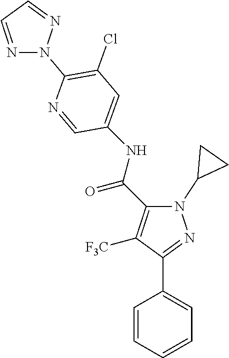 [SMILES: O=C(Nc1cnc(-n2nccn2)c(Cl)c1)c1c(C(F)(F)F)c(-c2ccccc2)nn1C1CC1] | 6-(2H-1,2,3- triazol-2-yl)-5- (trifluoromethyl) pyridin-3-amine | (ES, m/z): [M + H]⁺ 507.39, ¹H NMR (400 MHz, DMSO) δ 12.16 (br s, 1H), 9.07 (br s, 1H), 8.84 (d, J = 2.3 Hz, 1H), 8.21 (s, 2H), 7.60 (dd, J = 1.6, 7.7 Hz, 2H), 7.54-7.49 (m, 3H), 3.97-3.90 (m, 1H), 1.23- 1.18 (m, 2H), 1.10-1.04 (m, 2H) |
| I-191 | 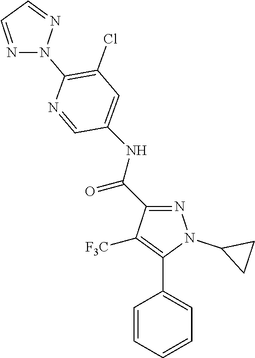 [SMILES: O=C(Nc1cnc(-n2nccn2)c(Cl)c1)c1nn(C2CC2)c(-c2ccccc2)c1C(F)(F)F] | ethyl 1- cyclopropyl-5- phenyl-4- (trifluoromethyl)- 1H-pyrazole-3- carboxylate and 5-chloro-6-(2H- 1,2,3-triazol-2- yl)pyridin-3- amine | (ES, m/z): [M + H]⁺ 473.84, ¹H NMR (400 MHz, DMSO) δ 11.08 (s, 1H), 8.99 (d, J = 2.3 Hz, 1H), 8.72 (d, J = 2.3 Hz, 1H), 8.19 (s, 2H), 7.62-7.57 (m, 5H), 3.65-3.58 (m, 1H), 1.19-1.14 (m, 2H), 0.93- 0.87 (m, 2H) |
| I-192 | 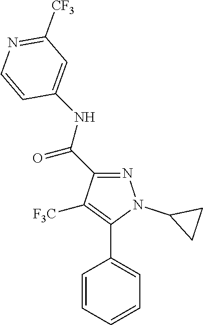 [SMILES: O=C(Nc1ccnc(C(F)(F)F)c1)c1nn(C2CC2)c(-c2ccccc2)c1C(F)(F)F] | ethyl 1- cyclopropyl-5- phenyl-4- (trifluoromethyl)- 1H-pyrazole-3- carboxylate and 2- (trifluoromethyl) pyridin-4-amine | (ES, m/z): [M + H]⁺ 440.34, ¹H NMR (400 MHz, DMSO) δ 11.98 (s, 1H), 8.76 (d, J = 5.5 Hz, 1H), 8.18 (d, J = 1.4 Hz, 1H), 7.90 (d, J = 4.5 Hz, 1H), 7.59 (dd, J = 1.9, 7.4 Hz, 2H), 7.54-7.50 (m, 3H), 3.89- 3.83 (m, 1H), 1.20-1.16 (m, 2H), 1.07-1.02 (m, 2H) |

### Example 28—Synthesis of 3-benzoyl-1-methyl-4-(trifluoromethyl)-N-(2-(trifluoromethyl)pyridin-4-yl)-1H-pyrazole-5-carboxamide (I-193)

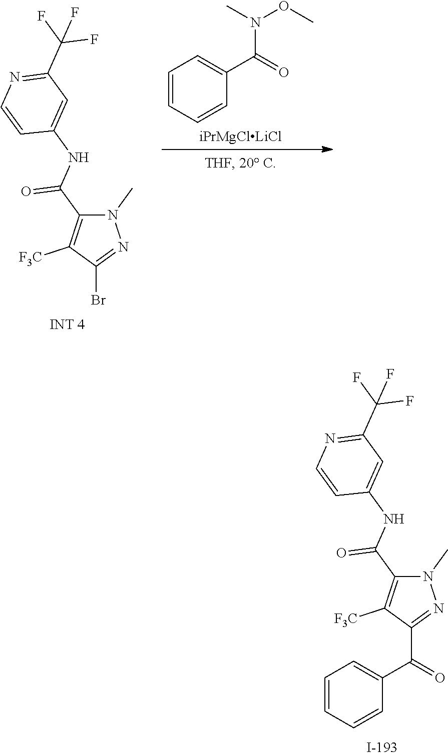 [SMILES: "iPrMgCl·LiCl" ; Cn1nc(Br)c(C(F)(F)F)c1C(=O)Nc1ccnc(C(F)(F)F)c1 ; CON(C)C(=O)c1ccccc1 ; Cn1nc(C(=O)c2ccccc2)c(C(F)(F)F)c1C(=O)Nc1ccnc(C(F)(F)F)c1]

Isopropylmagnesium chloride lithium chloride complex solution (1.3 M in THF, 0.14 mL, 0.176 mmol, 2.10 eq) was added dropwise to a solution of 3-bromo-1-methyl-4-(trifluoromethyl)-N-(2-(trifluoromethyl)pyridin-4-yl)-1H-pyrazole-5-carboxamide (35 mg, 0.0839 mmol, 1.0 eq) and N-methoxy-N-methylbenzamide (0.013 mL, 0.0839 mmol, 1.00 eq) in dry THF (1 mL) under an atmosphere of nitrogen. The reaction was stirred at rt for 30 minutes, then it was quenched with saturated aqueous NH₄Cl and stirred for 15 minutes. The crude was diluted with EtOAc and the organic phase was separated, dried (MgSO₄), filtered and concentrated under reduced pressure. The crude residue was purified by reverse phase HPLC give the title compound as an off-white solid (14 mg, 0.0304 mmol, 36%). (ES, m/z): [M+H]⁺ 443, ¹H NMR (400 MHz, DMSO) δ 11.95 (s, 1H), 8.75 (d, J=5.6 Hz, 1H), 8.17 (s, 1H), 8.08-8.04 (m, 2H), 7.87 (d, J=4.5 Hz, 1H), 7.74 (t, J=7.3 Hz, 1H), 7.60 (t, J=7.7 Hz, 2H), 4.05 (s, 3H).

### Example 29—Synthesis of 3-(1-(hydroxymethyl) isoquinolin-4-yl)-1-methyl-4-(trifluoromethyl)-N-(2-(trifluoromethyl)pyridin-4-yl)-1H-pyrazole-5-carboxamide (I-194)

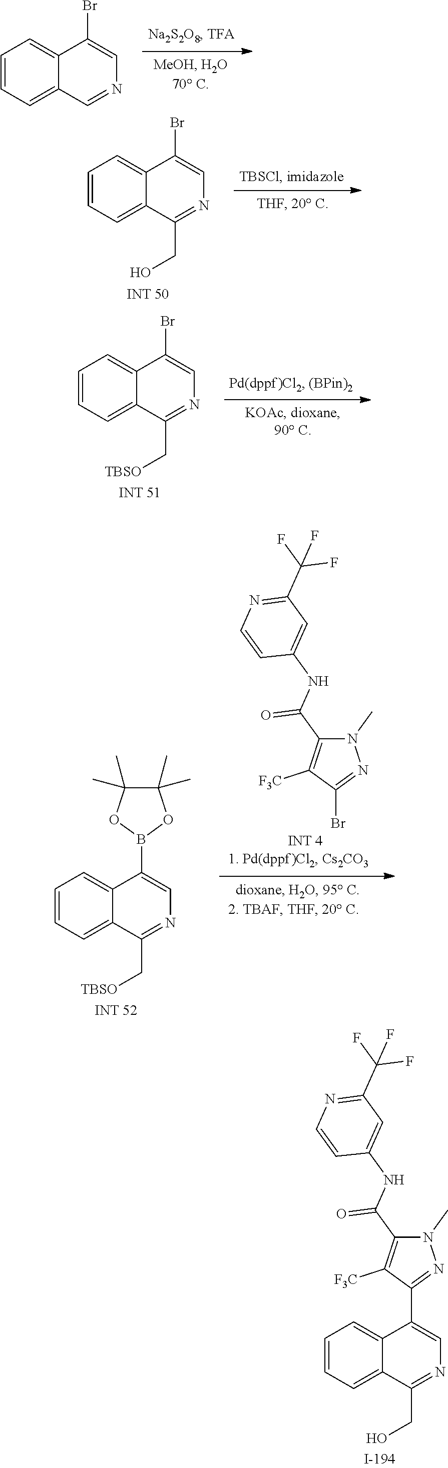 [SMILES: Brc1cncc2ccccc12 ; OCc1ncc(Br)c2ccccc12 ; CC(C)(C)[Si](C)(C)OCc1ncc(Br)c2ccccc12 ; CC1(C)OB(c2cnc(CO[Si](C)(C)C(C)(C)C)c3ccccc23)OC1(C)C ; Cn1nc(Br)c(C(F)(F)F)c1C(=O)Nc1ccnc(C(F)(F)F)c1 ; Cn1nc(-c2cnc(CO)c3ccccc23)c(C(F)(F)F)c1C(=O)Nc1ccnc(C(F)(F)F)c1]

Step 1: Synthesis of (4-bromoisoquinolin-1-yl) methanol (INT 50): TFA (0.48 mL, 6.24 mmol, 1.30 eq) was added dropwise to a solution of 4-bromoisoquinoline (1 g, 4.80 mmol, 1.00 eq) and sodium persulfate (2.28 g, 9.60 mmol, 2.00 eq) in MeOH (21 mL) and water (9 mL). The reaction tube was flushed with nitrogen, sealed, and stirred at 70° C. for 4 hours, then it was allowed to cool rt. The crude mixture was concentrated under reduced pressure. The residue was partitioned between DCM (50 mL) and saturated aqueous NaHCO₃ (10 mL). The aqueous layer was separated and extracted with DCM (10 mL) and the combined organic phases were washed with brine (5 mL), dried (MgSO₄), filtered and concentrated under reduced pressure. The crude residue was purified by column chromatography eluting with 0-100% EtOAc in cyclohexane to give the title compound as a yellow solid (260 mg, 1.09 mmol, 25%). ¹H NMR (400 MHz, CDCl₃) δ 8.66 (s, 1H), 8.24 (d, J=8.9 Hz, 1H), 7.94 (d, J=10.7 Hz, 1H), 7.86 (t, J=7.1 Hz, 1H), 7.73-7.68 (m, 1H), 5.22 (s, 2H), 4.68 (s, 1H)

Step 2: Synthesis of 4-bromo-1-(((tert-butyldimethylsilyl)oxy)methyl) isoquinoline (INT 51): A mixture of (4-bromoisoquinolin-1-yl) methanol (260 mg, 1.09 mmol, 1.00 eq), imidazole (149 mg, 2.18 mmol, 2.00 eq) and tert-butyldimethylsilyl chloride (329 mg, 2.18 mmol, 2.00 eq) in THF (7 mL) was stirred at rt for 72 hours. The reaction mixture was concentrated under reduced pressure. The residue was partitioned between EtOAc (30 mL) and water (10 mL), the aqueous phase was separated and extracted with EtOAc (10 mL). The combined organic phases were dried (MgSO₄), filtered and concentrated under reduced pressure. The crude residue was purified by column chromatography eluting with 0-30% EtOAc in cyclohexane to give the title compound as a yellow oil (168 mg, 0.477 mmol, 44%). ¹H NMR (400 MHz, CDCl₃) δ 8.55-8.54 (m, 1H), 8.40-8.37 (m, 1H), 8.13-8.09 (m, 1H), 7.74-7.69 (m, 1H), 7.62-7.57 (m, 1H), 5.15 (s, 2H), 0.80 (s, 9H), 0.01 (s, 6H).

Step 3: Synthesis of 1-(((tert-butyldimethylsilyl)oxy)methyl)-4-(4,4,5,5-tetramethyl-1,3,2-dioxaborolan-2-yl) isoquinoline (INT 52): Pd(dppf)Cl₂ (35 mg, 0.0468 mmol, 0.100 eq) was added to a degassed mixture of 4-bromo-1-(((tert-butyldimethylsilyl)oxy)methyl) isoquinoline (165 mg, 0.468 mmol, 1.00 eq), bis(pinacolato)diboron (131 mg, 0.515 mmol, 1.10 eq) and KOAc (138 mg, 1.40 mmol, 3.00 eq) in 1,4-dioxane (3 mL) and the reaction was stirred at 90° C. for 10 hours. The reaction mixture was filtered through filter paper and the filtrate was concentrated under reduced pressure. The crude residue was purified by column chromatography eluting with 0-100% EtOAc in cyclohexane to give the title compound as a yellow oil (120 mg, 0.300 mmol, 64%). ¹H NMR (400 MHz, CDCl₃) δ 8.81 (s, 1H), 8.65-8.62 (m, 1H), 8.43-8.40 (m, 1H), 7.69-7.64 (m, 1H), 7.56-7.51 (m, 1H), 5.23 (s, 2H), 1.38 (s, 12H), 0.81-0.81 (m, 9H), 0.00 (s, 6H).

Step 4: Synthesis of 3-(1-(hydroxymethyl) isoquinolin-4-yl)-1-methyl-4-(trifluoromethyl)-N-(2-(trifluoromethyl)pyridin-4-yl)-1H-pyrazole-5-carboxamide (I-194): Pd(dppf)Cl₂ (5.3 mg, 7.19 μmol, 0.1000 eq) was added to a degassed solution of tert-butyl-dimethyl-[[4-(4,4,5,5-tetramethyl-1,3,2-dioxaborolan-2-yl)-1-isoquinolyl]methoxy]silane (37 mg, 0.0935 mmol, 1.30 eq), 5-bromo-2-methyl-4-(trifluoromethyl)-N-[2-(trifluoromethyl)-4-pyridyl]pyrazole-3-carboxamide (30 mg, 0.0719 mmol, 1.00 eq) and Cs₂CO₃ (70 mg, 0.216 mmol, 3.00 eq) in a mixture of 1,4-dioxane (1 mL) and water (0.15 mL) and the reaction was stirred at 95° C. for 4 hours. After cooling down, the crude mixture was filtered through a pad of Celite® and the filtrate was concentrated under reduced pressure. The residue was taken up in THF (1 mL) and TBAF (1 M in THF, 0.072 mL, 0.0719 mmol, 1.20 eq) was added. The mixture was stirred for 3 hours at rt, then it was partitioned between DCM and water. The aqueous phase was separated and extracted with DCM. The combined organic phases were dried (MgSO₄), filtered and concentrated under reduced pressure. The crude residue was purified by reverse phase HPLC to give the title compound as an off-white solid (7.2 mg, 0.014 mmol, 20%). (ES, m/z): [M+H]⁺ 496.2, ¹H NMR (400 MHz, DMSO) δ d 12.00 (s, 1H), 8.74 (d, J=4.4 Hz, 1H), 8.50-8.49 (m, 1H), 8.42 (s, 1H), 8.20-8.19 (m, 1H), 7.91-7.75 (m, 3H), 5.55 (t, J=5.6 Hz, 1H), 5.15-5.11 (m, 2H), 4.09 (s, 3H).

### Example 30—Synthesis of 3-(imidazo[1,2-a]pyridin-3-yl)-1-methyl-4-(trifluoromethyl)-N-(2-(trifluoromethyl)pyridin-4-yl)-1H-pyrazole-5-carboxamide (I-195)

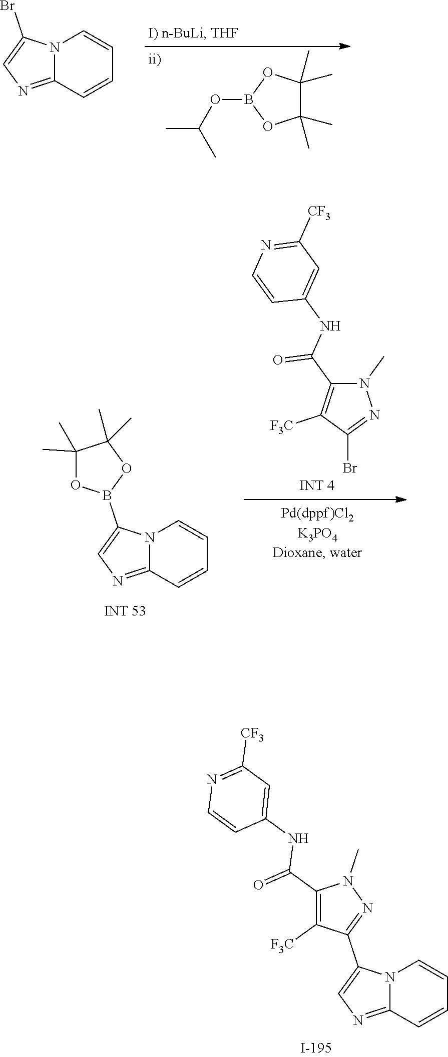 [SMILES: Brc1cnc2ccccn12 ; CC(C)OB1OC(C)(C)C(C)(C)O1 ; CC1(C)OB(c2cnc3ccccn23)OC1(C)C ; Cn1nc(Br)c(C(F)(F)F)c1C(=O)Nc1ccnc(C(F)(F)F)c1 ; Cn1nc(-c2cnc3ccccn23)c(C(F)(F)F)c1C(=O)Nc1ccnc(C(F)(F)F)c1]

Step 1: Synthesis of 3-(4,4,5,5-tetramethyl-1,3,2-dioxaborolan-2-yl) imidazo[1,2-a]pyridine (INT 53): 2.4 M n-BuLi in hexanes (0.48 mL, 1.14 mmol, 1.50 eq) was added dropwise to a stirred mixture of 3-bromoimidazo[1,2-a]pyridine (150 mg, 0.761 mmol, 1.00 eq) in THF (7.5 mL) cooled to −78° C. and the reaction was stirred for 5 mins. 2-Isopropoxy-4,4,5,5-tetramethyl-1,3,2-dioxaborolane (0.23 mL, 1.14 mmol, 1.50 eq) was added and the reaction was allowed to warm to room temperature and stirred overnight. The reaction mixture was concentrated under reduced pressure and immediately used in the next reaction.

Step 2: Synthesis of 3-(imidazo[1,2-a]pyridin-3-yl)-1-methyl-4-(trifluoromethyl)-N-(2-(trifluoromethyl)pyridin-4-yl)-1H-pyrazole-5-carboxamide (I-195): Pd(dppf)Cl₂ (16 mg, 0.0192 mmol, 0.100 eq) was added to a degassed solution of 3-bromo-1-methyl-4-(trifluoromethyl)-N-(2-(trifluoromethyl)pyridin-4-yl)-1H-pyrazole-5-carboxamide (80 mg, 0.192 mmol, 1.00 eq), 3-(4,4,5,5-tetramethyl-1,3,2-dioxaborolan-2-yl) imidazo[1,2-a]pyridine (47 mg, 0.192 mmol, 1.00 eq) and K₃PO₄ (133 mg, 0.575 mmol, 3.00 eq) in 1,4-dioxane (2 mL) and water (0.2 mL) and the reaction was stirred at 85° C. overnight. The reaction mixture was partitioned between EtOAc, water and brine. The organic phase was dried (MgSO₄), filtered and concentrated under reduced pressure. The crude residue was purified by column chromatography eluting with 0-100% EtOAc in cyclohexane, followed by EtOAc:EtOH 3:1 to give impure material, which was further purified by reverse phase HPLC to give the title compound as an off white solid (9.9 mg, 0.0212 mmol, 11%). (ES, m/z): [M+H]⁺ 455.0, ¹H NMR (400 MHz, DMSO) δ 12.02-12.01 (m, 1H), 9.11 (d, J=7.1 Hz, 1H), 8.73 (d, J=3.3 Hz, 1H), 8.17 (s, 1H), 7.90 (s, 1H), 7.86-7.84 (m, 1H), 7.75 (d, J=9.1 Hz, 1H), 7.47-7.41 (m, 1H), 7.16-7.11 (m, 1H), 4.06 (s, 3H).

### Example 31—Synthesis of methyl 1-methyl-3-phenoxy-4-(trifluoromethyl)-1H-pyrazole-5-carboxylate

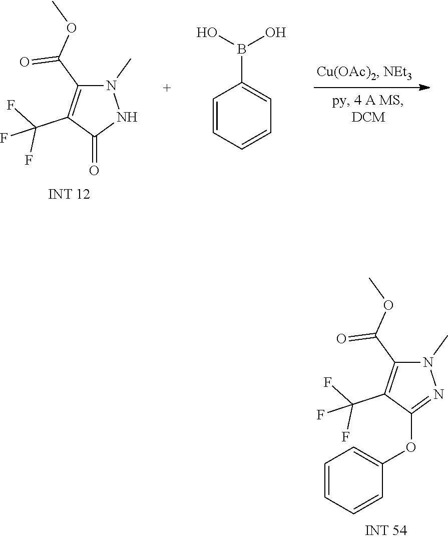 [SMILES: COC(=O)c1c(C(F)(F)F)c(=O)[nH]n1C ; OB(O)c1ccccc1 ; COC(=O)c1c(C(F)(F)F)c(Oc2ccccc2)nn1C]

Step 1: Synthesis of methyl 1-methyl-3-phenoxy-4-(trifluoromethyl)-1H-pyrazole-5-carboxylate (INT 54): NEt₃ (0.062 mL, 0.446 mmol, 1.00 eq) and pyridine (0.072 mL, 0.892 mmol, 2.00 eq) were added to a stirred mixture of methyl 2-methyl-5-oxo-4-(trifluoromethyl)-2,5-dihydro-1H-pyrazole-3-carboxylate (100 mg, 0.446 mmol, 1.00 eq), phenylboronic acid (82 mg, 0.669 mmol, 1.50 eq), Cu(OAc)₂ (122 mg, 0.669 mmol, 1.50 eq) and 4 Å mol sieves (200 mg) in DCM (10 mL). The reaction was stirred open to the air at rt for 16 h. The reaction was filtered through Celite®, diluted with DCM and washed with saturated aqueous NH₄Cl, water and brine. The organic phase was dried and concentrated and the crude residue was purified by column chromatography eluting with 0-100% EtOAc in cyclohexane to give the title product (40 mg, 0.133 mmol, 30%) as colourless oil. (ES, m/z): [M+H]⁺ 301.2, ¹H NMR (400 MHz, CDCl₃) δ 7.36-7.31 (m, 2H), 7.16-7.08 (m, 3H), 4.05 (s, 3H), 3.97 (s, 3H).

### Example 32 Synthesis of 3-(6-amino-5-fluoropyridin-2-yl)-1-methyl-4-(trifluoromethyl)-N-(2-(trifluoromethyl)pyridin-4-yl)-1H-pyrazole-5-carboxamide (I-196)

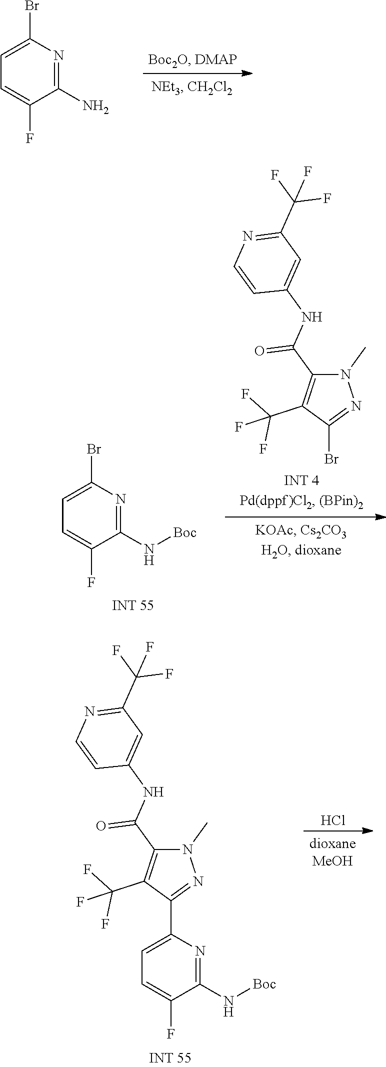 [SMILES: Nc1nc(Br)ccc1F ; CC(C)(C)OC(=O)Nc1nc(Br)ccc1F ; Cn1nc(Br)c(C(F)(F)F)c1C(=O)Nc1ccnc(C(F)(F)F)c1 ; Cn1nc(-c2ccc(F)c(NC(=O)OC(C)(C)C)n2)c(C(F)(F)F)c1C(=O)Nc1ccnc(C(F)(F)F)c1 ; Cl]

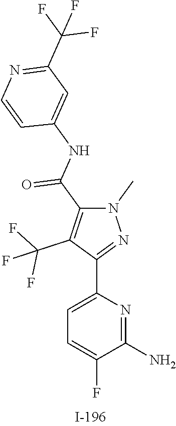 [SMILES: Cn1nc(-c2ccc(F)c(N)n2)c(C(F)(F)F)c1C(=O)Nc1ccnc(C(F)(F)F)c1]

Step 1: Synthesis of tert-butyl (6-bromo-3-fluoropyridin-2-yl) carbamate (INT 55): Boc₂O (0.36 mL, 1.57 mmol, 1.20 eq) in DCM (4 mL) was added dropwise to a stirred solution of 6-bromo-3-fluoropyridin-2-amine (250 mg, 1.31 mmol, 1.00 eq), DMAP (32 mg, 0.262 mmol, 0.200 eq) and NEt₃ (0.24 mL, 1.70 mmol, 1.30 eq) in DCM (6 mL) and the reaction was stirred at rt for 36 h. The reaction was diluted with DCM and washed with saturated aqueous NaHCO₃ and brine. The organic phase was dried (MgSO₄) and concentrated. The crude residue was purified by column chromatography eluting with 0-100% EtOAc in cyclohexane to give the title compound (300 mg, 1.03 mmol, 78%) as a white solid. (ES, m/z): [M+H-tBu]⁺ 235.1/237.1, ¹H NMR (400 MHz, CDCl₃) δ 7.46 (dd, J=3.2, 8.5 Hz, 1H), 7.37 (dd, J=8.3, 8.3 Hz, 1H), 1.43 (s, 9H), (NH missing).

Step 2: Synthesis of tert-butyl (3-fluoro-6-(1-methyl-4-(trifluoromethyl)-5-((2-(trifluoromethyl)pyridin-4-yl) carbamoyl)-1H-pyrazol-3-yl) pyridin-2-yl) carbamate (INT 56): Pd(dppf)Cl₂ (27 mg, 0.0360 mmol, 0.100 eq) was added to a degassed suspension of bis(pinacolato)diboron (100 mg, 0.396 mmol, 1.10 eq), 5-bromo-2-methyl-4-(trifluoromethyl)-N-[2-(trifluoromethyl)-4-pyridyl]pyrazole-3-carboxamide (150 mg, 0.360 mmol, 1.00 eq) and KOAc (106 mg, 1.08 mmol, 3.00 eq) in 1,4-dioxane (3 mL) and the reaction was heated at 135° C. in the microwave for 4 h. tert-Butyl (6-bromo-3-fluoropyridin-2-yl) carbamate (105 mg, 0.360 mmol, 1.00 eq), Cs₂CO₃ (352 mg, 1.08 mmol, 3.00 eq), Pd(dppf)Cl₂ (27 mg, 0.0360 mmol, 0.100 eq) and water (0.3 mL) were added and the reaction was heated at 95° C. for 16 h. The reaction mixture was cooled to rt and partitioned between EtOAc and saturated aqueous NaHICO₃. The organic phase was dried (MgSO₄) and concentrated and the crude residue was purified by column chromatography eluting with 0-100% EtOAc in cyclohexane to give the title compound as semi-pure yellow oil (˜40% purity, 100 mg) which was used directly in the next step. (ES, m/z): [M+H-Boc]⁺ 449.4 in LCMS

Step 3: Synthesis of 3-(6-amino-5-fluoropyridin-2-yl)-1-methyl-4-(trifluoromethyl)-N-(2-(trifluoromethyl)pyridin-4-yl)-1H-pyrazole-5-carboxamide (I-196): 4 M HCl in 1,4-dioxane (0.18 mL, 0.729 mmol, 10.0 eq) was added to a stirred solution of tert-butyl N-[3-fluoro-6-[1-methyl-4-(trifluoromethyl)-5-[[2-(trifluoromethyl)-4-pyridyl]carbamoyl]pyrazol-3-yl]-2-pyridyl]carbamate (40%, 100 mg, 0.0729 mmol, 1.00 eq) in MeOH (3 mL) and the reaction was stirred at rt for 16 h. The reaction was concentrated to dryness and purified by reverse phase HPLC to give the title compound as an off-white solid (13.0 mg, 0.03 mmol, 40%). (ES, m/z): [M+H]⁺ 449.2, ¹H NMR (400 MHz, DMSO) δ 11.89-11.89 (m, 1H), 8.74 (d, J=5.0 Hz, 1H), 8.17 (s, 1H), 7.87 (d, J=4.4 Hz, 1H), 7.45 (dd, J=8.0, 11.2 Hz, 1H), 6.95 (dd, J=2.9, 8.1 Hz, 1H), 6.24 (s, 2H), 3.95 (s, 3H).

### Example 33—Synthesis of Additional Compounds

Additional compounds synthesized using the route described above are provided in Table 15.

TABLE 15

| Compound | | | |
|---|---|---|---|
| No. | Structure | Coupling partner | Analytical data |
| I-197 | 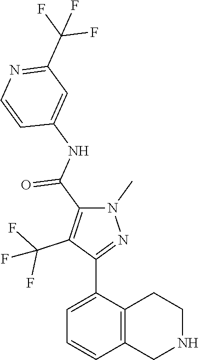 [SMILES: Cn1nc(-c2cccc3c2CCNC3)c(C(F)(F)F)c1C(=O)Nc1ccnc(C(F)(F)F)c1] | tert-butyl 5- (4,4,5,5- tetramethyl-1,3,2- dioxaborolan-2- yl)-3,4- dihydroisoquinoline- 2(1H)- carboxylate | (ES, m/z): [M + H]⁺ 470.2, ¹H NMR (400 MHz, DMSO) δ 11.86 (broad s, 1H), 8.73 (d, J = 5.4 Hz, IH), 8.18 (d, J = 1.6 Hz, 1H), 7.89 − 7.87 (m, 1H), 7.25 − 7.13 (m, 2H), 7.07 (d, J = 7.0 Hz, 1H), 3.99 (s, 3H), 3.95 (s, 2H), 2.96 − 2.90 (m, 2H), 2.51 − 2.50 (m, 2H). exchangeable proton not seen. |
| I-198 | 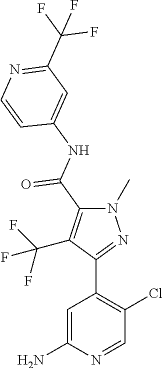 [SMILES: Cn1nc(-c2cc(N)ncc2Cl)c(C(F)(F)F)c1C(=O)Nc1ccnc(C(F)(F)F)c1] | tert-butyl (5- chloro-4-(4,4,5,5- tctramethyl-1,3,2- dioxaborolan-2- yl)pyridin-2- yl)carbamate | (ES, m/z): [M + H]⁺ 465.2, ¹H NMR (400 MHz, DMSO) δ 11.93 (s, 1H), 8.72 − 8.72 (m, 1H), 8.17 (s, 1H), 8.05 (s, 1H), 7.87 (s, 1H), 6.47 (s, 1H), 6.38 (s, 2H), 4.01 (s, 3H) |
| I-199 | 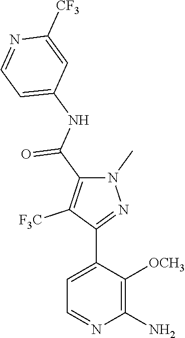 [SMILES: COc1c(-c2nn(C)c(C(=O)Nc3ccnc(C(F)(F)F)c3)c2C(F)(F)F)ccnc1N] | tert-butyl (3- methoxy-4-(1- methyl-4- (trifluoromethyl)- 5-((2- (trifluoromethyl) pyridin-4- yl)carbamoyl)-1H- pyrazol-3- yl)pyridin-2- yl)carbamate | (ES, m/z): [M + H]⁺ 461.2, ¹H NMR (400 MHz, DMSO) δ 11.92 (s, 1H), 8.81 (d, J = 5.6 Hz, 1H), 8.24 (d, J = 1.5 Hz, 1H), 7.95 (dd, J = 1.4, 5.4 Hz, 1H), 7.81 (d, J = 5.3 Hz, 1H), 6.53 (d, J = 5.3 Hz, 1H), 6.32 (s, 2H), 4.07 (s, 3H), 3.58 (s, 3H) |
| I-200 | 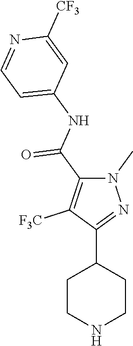 [SMILES: Cn1nc(C2CCNCC2)c(C(F)(F)F)c1C(=O)Nc1ccnc(C(F)(F)F)c1] | tert-butyl 4-(1- methyl-4- (trifluoromethyl)- 5-((2- (trifluoromethyl) pyridin-4- yl)carbamoyl)-1H- pyrazol-3- yl)piperidine-1 − carboxylate | (ES, m/z): [M + H]⁺ 422.2, ¹H NMR (400 MHz, CDCl₃) δ 8.70 (d, J = 5.5 Hz, 1H), 8.30 (s, 1H − formate), 8.14 (d, J = 1.7 Hz, 1H), 7.83 (dd, J = 5.5, 1.7 Hz, 1H), 3.88 (s, 3H), 3.16 (br d, J = 12.5 Hz, 2H), 2.97-2.87 (1H, m), 2.76 (2H, dt, J = 2.6, 12.2 Hz), 1.87- 1.69 (4H, m) amine and amide NH not visible |
| I-201 | 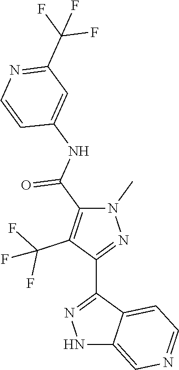 [SMILES: Cn1nc(-c2n[nH]c3cnccc23)c(C(F)(F)F)c1C(=O)Nc1ccnc(C(F)(F)F)c1] | tert-butyl 3-(1- methyl-4- (trifluoromethyl)- 5-((2- (trifluoromethyl) pyridin-4- yl)carbamoyl)-1H- pyrazol-3-yl)-1H- pyrazolo[3,4- c]pyridine-1- carboxylate | (ES, m/z): [M + H]⁺ 456.2, ¹H NMR (400 MHz, DMSO) δ 14.03 (s, 1H), 11.96 (s, 1H), 9.14 (s, 1H), 8.76 (d, J = 5.3 Hz, 1H), 8.37 (d, J = 5.6 Hz, 1H), 8.20 (s, 1H), 8.09 (d, J = 5.8 Hz, 1H), 7.89 (d, J = 4.5 Hz, 1H), 4.07 (s, 3H). |
| I-239 | 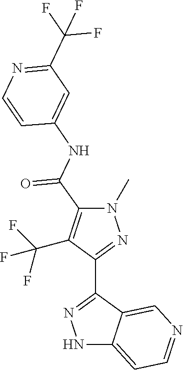 [SMILES: Cn1nc(-c2n[nH]c3ccncc23)c(C(F)(F)F)c1C(=O)Nc1ccnc(C(F)(F)F)c1] | tert-butyl 3-(1- methyl-4- (trifluoromethyl)- 5-((2- (trifluoromethyl) pyridin-4- yl)carbamoyl)- 1H-pyrazol-3- yl)-1H- pyrazolo[4,3- c]pyridine-1- carboxylate | (ES, m/z): [M + H]⁺ 456.2, ¹H NMR (400 MHz, DMSO) 13.85 (s, 1H), 9.48 (d, J = 1.0 Hz, 1H), 8.73 (d, J-5.6 Hz, 1H), 8.43 (d, J = 6.1 Hz, 1H), 8.30 (s, 1H), 8.19 (d, J = 1.3 Hz, 1H), 7.88 (d, J-4.3 Hz, 1H), 7.62 (dd, J = 1.0, 6.1 Hz, 1H), 4.08 (s, 3H). |
| I-242 | 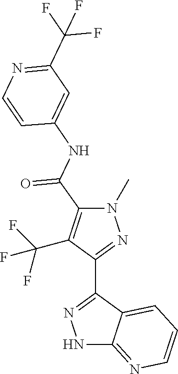 [SMILES: Cn1nc(-c2n[nH]c3ncccc23)c(C(F)(F)F)c1C(=O)Nc1ccnc(C(F)(F)F)c1] | tert-butyl 3-(1- methyl-4- (trifluoromethyl)- 5-((2- (trifluoromethyl) pyridin-4- yl)carbamoyl)- 1H-pyrazol-3- yl)-1H- pyrazolo[3,4- b]pyridine-1- carboxylate | (ES, m/z): [M + H]⁺ 456.3, ¹H NMR (400 MHz, DMSO) 14.03 (s, 1H), 11.96 (s, 1H), 8.76 − 8.73 (m, 1H), 8.62 (dd, J = 1.5, 4.5 Hz, 1H), 8.56 (dd, J = 1.5, 8.1 Hz, 1H), 8.19 (s, 1H), 7.87 (s, 1H), 7.34 (dd, J = 4.5, 8.1 Hz, 1H), 4.05 (s, 3H). |

### Example 34—Synthesis of 1-methyl-3-(7H-pyrrolo[2,3-d]pyrimidin-5-yl)-4-(trifluoromethyl)-N-(2-(trifluoromethyl)pyridin-4-yl)-1H-pyrazole-5-carboxamide (I-202)

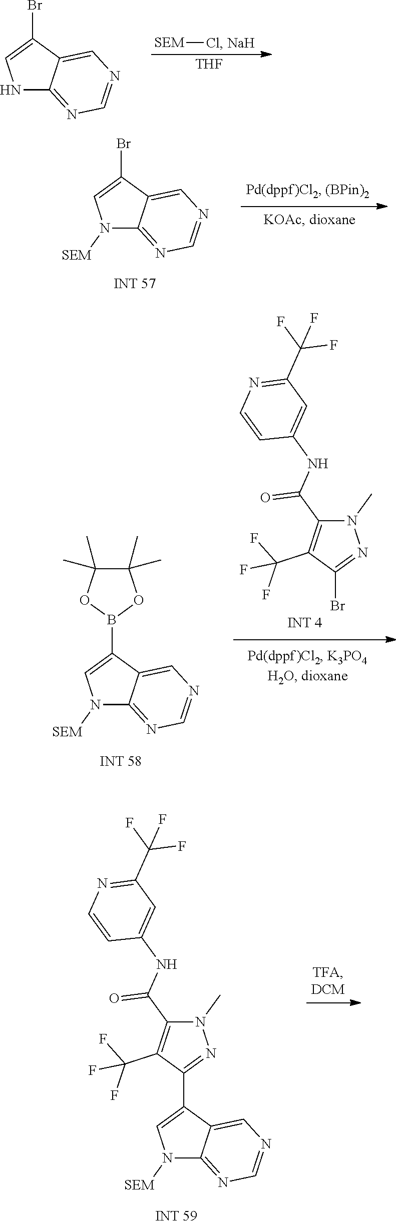

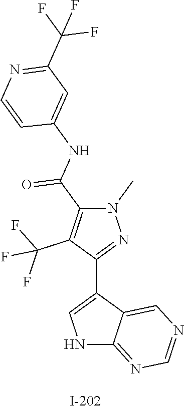 [SMILES: Cn1nc(-c2c[nH]c3ncncc23)c(C(F)(F)F)c1C(=O)Nc1ccnc(C(F)(F)F)c1]

Step 1: Synthesis of 5-bromo-7-((2-(trimethylsilyl) ethoxy)methyl)-7H-pyrrolo[2,3-d]pyrimidine (INT 57): NaH (60%, 91 mg, 2.27 mmol, 1.50 eq) was added to a stirred mixture of 5-bromo-7H-pyrrolo[2,3-d]pyrimidine (300 mg, 1.51 mmol, 1.00 eq) in THF (5 mL) at 0° C. and the reaction was stirred at rt for 30 min. 2-(Trimethylsilyl) ethoxymethyl chloride (0.32 mL, 1.82 mmol, 1.20 eq) was added dropwise and the mixture was allowed to warm to rt and stirred for 16 h. The reaction was quenched with saturated aqueous NH₄Cl and diluted with EtOAc. The organic phase was separated, washed with saturated aqueous NaHCO₃ and brine, dried (MgSO₄) and concentrated. The crude residue was purified by column chromatography eluting with 0-100% EtOAc in cyclohexane to give the title compound as a colourless oil (180 mg, 0.55 mmol, 36%). (ES, m/z): [M+H]⁺ 327.9/329.9, ¹H NMR (400 MHz, CDCl₃) δ 8.94 (d, J=2.8 Hz, 2H), 7.39 (s, 1H), 5.63 (s, 2H), 3.56-3.51 (m, 2H), 0.94-0.89 (m, 2H), −0.05 (s, 9H).

Step 2: Synthesis of 5-(4,4,5,5-tetramethyl-1,3,2-dioxaborolan-2-yl)-7-((2-(trimethylsilyl) ethoxy)methyl)-7H-pyrrolo[2,3-d]pyrimidine (INT 58): The title compound was synthesized following based on procedures described in Example 2, to give the title compound as colorless oil (80 mg, 0.21 mmol, 39%). (ES, m/z): [M+H]⁺ 376.2, ¹H NMR (400 MHz, CDCl₃) δ 9.25 (s, 1H), 8.92 (s, 1H), 7.78 (s, 1H), 5.64 (s, 2H), 3.55-3.50 (m, 2H), 1.37 (s, 12H), 0.93-0.88 (m, 2H), −0.06 (s, 9H).

Step 3: Synthesis of 1-methyl-4-(trifluoromethyl)-N-(2-(trifluoromethyl)pyridin-4-yl)-3-(7-((2-(trimethylsilyl) ethoxy)methyl)-7H-pyrrolo[2,3-d]pyrimidin-5-yl)-1H-pyrazole-5-carboxamide (INT 59): The title compound was synthesized following based on procedures described in Example 2 and isolated as a waxy solid (55 mg, 0.094 mmol, 49%). (ES, m/z): [M+H]⁺ 586.2, ¹H NMR (400 MHz, CDCl₃) δ 9.19 (s, 1H), 8.96 (s, 1H), 8.76-8.74 (m, 1H), 8.08 (s, 1H), 7.84-7.83 (m, 1H), 7.63 (s, 1H), 5.68 (s, 2H), 4.19 (s, 3H), 3.65-3.54 (m, 2H), 0.99-0.89 (m, 2H), −0.06 (s, 9H); NH not visible.

Step 4: Synthesis of 1-methyl-3-(7H-pyrrolo[2,3-d]pyrimidin-5-yl)-4-(trifluoromethyl)-N-(2-(trifluoromethyl)pyridin-4-yl)-1H-pyrazole-5-carboxamide (I-202): A solution of 1-methyl-4-(trifluoromethyl)-N-(2-(trifluoromethyl)pyridin-4-yl)-3-(7-((2-(trimethylsilyl) ethoxy)methyl)-7H-pyrrolo[2,3-d]pyrimidin-5-yl)-1H-pyrazole-5-carboxamide (55 mg, 0.0939 mmol, 1.00 eq) and TFA (0.30 mL, 3.92 mmol, 41.7 eq) in DCM (5 mL) was stirred at rt for 6 h. The reaction mixture was concentrated under reduced pressure and the crude residue was purified by reverse phase HPLC to give the title compound as an off-white solid (6.9 mg, 0.015 mmol, 16%). (ES, m/z): [M+H]⁻ 456.2, ¹H NMR (400 MHz, DMSO) δ 12.57 (br s, 1H), 11.97-11.93 (m, 1H), 9.41 (s, 1H), 8.87 (s, 1H), 8.75-8.73 (m, 1H), 8.18 (s, 1H), 7.87 (d, J=2.8 Hz, 1H), 7.76 (s, 1H), 4.02 (s, 3H).

### Example 35—Synthesis of 1-methyl-3-(2-(2,2,2-trifluoroethyl)phenyl)-N-(2-(trifluoromethyl)pyridin-4-yl)-1H-pyrazole-5-carboxamide (I-203)

Step 1: Synthesis of 4,4,5,5-tetramethyl-2-(2-(2,2,2-trifluoroethyl)phenyl)-1,3,2-dioxaborolane (INT 60).

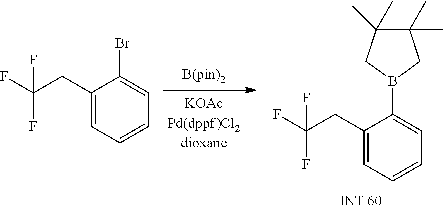 [SMILES: FC(F)(F)Cc1ccccc1Br ; CC1(C)CB(c2ccccc2CC(F)(F)F)CC1(C)C]

Pd(dppf)Cl₂ (83 mg, 0.102 mmol, 0.0623 eq) was added to a degassed suspension of KOAc (480 mg, 4.89 mmol, 3.00 eq), 1-bromo-2-(2,2,2-trifluoroethyl)benzene (390 mg, 1.63 mmol, 1.00 eq) and bis(pinacolato)diboron (620 mg, 2.44 mmol, 1.50 eq) in 1,4-dioxane (4.0 mL) and the reaction was stirred under one atmosphere of nitrogen at 100° C. for 16 hours. The mixture was cooled to rt, filtered through Celite® and the filter cake was washed with 1,4-dioxane. The filtrate was concentrated under reduced pressure and the crude residue was purified by column chromatography eluting with 0-20% EtOAc in cyclohexane to give the title compound as a clear oil (237 mg, 0.80 mmol, 49%). ¹H NMR (400 MHz, CDCl₃) δ 7.85 (dd, J=1.3, 7.3 Hz, 1H), 7.42 (dt, J=1.5, 7.5 Hz, 1H), 7.34 (dd, J=1.2, 7.5 Hz, 1H), 7.29 (d, J=8.3 Hz, 1H), 3.84 (q, J=11.0 Hz, 2H), 1.34 (s, 12H).

Step 2: Synthesis of methyl 3-bromo-1-methyl-1H-pyrazole-5-carboxylate (INT 61).

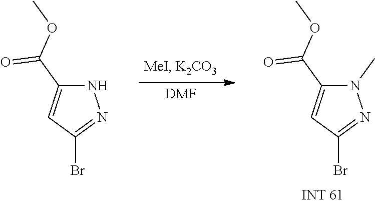

MeI (336 μL, 5.40 mmol, 1.11 eq) was added to a suspension of K₂CO₃ (935 mg, 6.77 mmol, 1.39 eq) and methyl 3-bromo-1H-pyrazole-5-carboxylate (1,000 mg, 4.88 mmol, 1.00 eq) in DMF (10 mL) and the reaction was stirred at 45° C. for 1 hour. The suspension was filtered, the filtrate was concentrated under reduced pressure and the residue was partitioned between water and EtOAc. The aqueous phase was separated and further extracted with EtOAc. The combined organic layers were dried (MgSO₄) and concentrated. The crude product was purified by column chromatography eluting with 5-25% EtOAc in cyclohexane to give the title compound as a colourless oil (530 mg, 2.42 mmol, 50%). ¹H NMR (400 MHz, CDCl₃) δ 6.81 (s, 1H), 4.15 (s, 3H), 3.89 (s, 3H).

Step 3: Synthesis of methyl 1-methyl-3-(2-(2,2,2-trifluoroethyl)phenyl)-1H-pyrazole-5-carboxylate (INT 63).

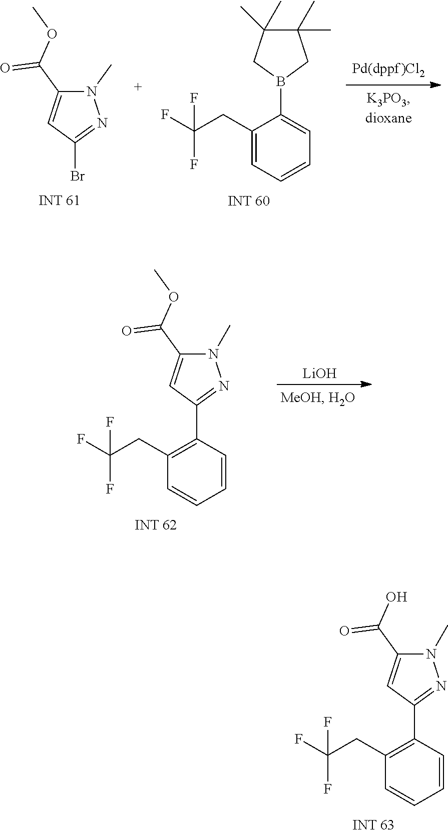 [SMILES: COC(=O)c1cc(Br)nn1C ; CC1(C)CB(c2ccccc2CC(F)(F)F)CC1(C)C ; COC(=O)c1cc(-c2ccccc2CC(F)(F)F)nn1C ; "LiOH" ; Cn1nc(-c2ccccc2CC(F)(F)F)cc1C(=O)O]

Pd(dppf)Cl₂ (66 mg, 0.0809 mmol, 0.0976 eq) was added to a degassed suspension of K₃PO₄ (556 mg, 2.41 mmol, 2.91 eq), methyl 3-bromo-1-methyl-1H-pyrazole-5-carboxylate (183 mg, 0.84 mmol, 1.01 eq) and 4,4,5,5-tetramethyl-2-(2-(2,2,2-trifluoroethyl)phenyl)-1,3,2-dioxaborolane (237 mg, 0.828 mmol, 1.00 eq) in 1,4-dioxane (5 mL) and water (0.50 mL) and the reaction was stirred at 100° C. for 5 h. The solvent was removed under reduced pressure and the residue was partitioned between water and EtOAc. The aqueous phase was separated and further extracted with EtOAc. The combined organic layers were dried (MgSO₄) and concentrated. The crude residue was purified by column chromatography eluting with 5-100% EtOAc in cyclohexane to give the title compound as a clear oil (219 mg, 0.69 mmol, 84%). ¹H NMR (400 MHz, CDCl₃) δ 7.51-7.48 (m, 1H), 7.44-7.40 (m, 1H), 7.41-7.33 (m, 2H), 6.97-6.96 (m, 1H), 4.24 (s, 3H), 3.92 (s, 3H), 3.81 (q, J=10.9 Hz, 2H).

1.3 M aqueous LiOH (0.3 mL, 0.402 mmol, 1.20 eq) was added to a solution of methyl 1-methyl-3-(2-(2,2,2-trifluoroethyl)phenyl)-1H-pyrazole-5-carboxylate (100 mg, 0.335 mmol, 1.00 eq) in MeOH (3 mL) and the reaction was stirred at 40° C. for 4 hours. The solvent was removed under reduced pressure to give a white solid, which was used in the next reaction without further purification.

Step 4: Synthesis of 1-methyl-3-(2-(2,2,2-trifluoroethyl)phenyl)-N-(2-(trifluoromethyl)pyridin-4-yl)-1H-pyrazole-5-carboxamide (I-203).

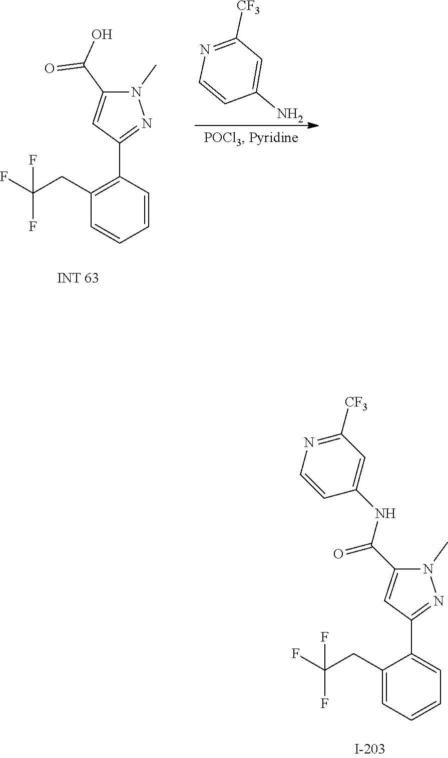

The title compound was synthesized from INT 63 based on procedures described in Example 8 and isolated by reverse phase HPLC as an off-white solid (65 mg, 0.15 mmol, 46%). (ES, m/z): [M+H]⁺ 429.5, ¹H NMR (400 MHz, DMSO) δ 10.93 (s, 1H), 8.71 (d, J=5.5 Hz, 1H), 8.28 (d, J=1.8 Hz, 1H), 8.02 (dd, J=2.0, 5.5 Hz, 1H), 7.60 (d, J=7.8 Hz, 1H), 7.51-7.41 (m, 3H), 7.41 (s, 1H), 4.18 (s, 3H), 4.16 (q, J=11.4 Hz, 2H).

### Example 36—Synthesis of 4-cyclopropyl-1-methyl-5-phenyl-N-(2-(trifluoromethyl)pyridin-4-yl)-1H-pyrazole-3-carboxamide (I-204)

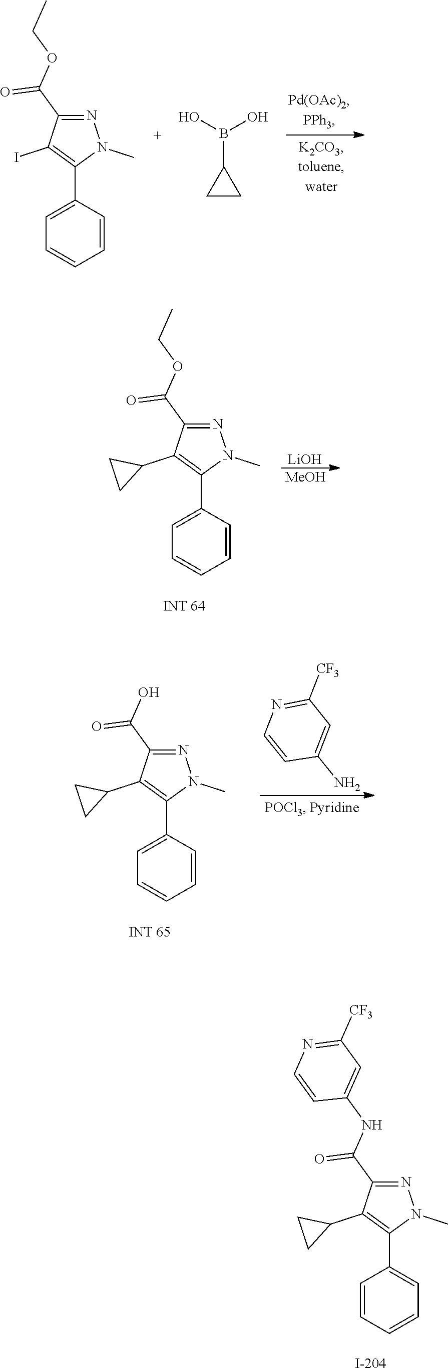

Step 1: Synthesis of ethyl 4-cyclopropyl-1-methyl-5-phenyl-1H-pyrazole-3-carboxylate (INT 64): Pd(OAc)₂ (9.5 mg, 0.0421 mmol, 0.100 eq) was added to a degassed suspension of cyclopropylboronic acid (57 mg, 0.664 mmol, 1.58 eq), ethyl 4-iodo-1-methyl-5-phenyl-1H-pyrazole-3-carboxylate (150 mg, 0.421 mmol, 1.00 eq), K₂CO₃ (128 mg, 0.927 mmol, 2.20 eq) and PPh₃ (49 mg, 0.187 mmol, 0.444 eq) in toluene (3 mL) and water (0.30 mL). The vial was sealed and stirred at 140° C. in a microwave for 90 mins. The reaction was cooled to rt and the solvent was removed under reduced pressure. The crude was partitioned between water and EtOAc. The aqueous layer was separated and extracted with EtOAc. The combined organic phases were dried (MgSO₄) and evaporated under reduced pressure, the crude residue was purified by column chromatography eluting with 5-40% EtOAc in cyclohexane to give the title compound as a clear glass (54 mg, 0.19 mmol, 46%). ¹H NMR (400 MHz, CDCl₃) δ 7.49-7.46 (m, 3H), 7.33-7.29 (m, 2H), 4.45 (q, J=7.1 Hz, 2H), 3.75-3.74 (m, 3H), 2.00-1.92 (m, 1H), 1.43 (t, J=7.3 Hz, 3H), 0.69-0.64 (m, 2H), 0.23-0.18 (m, 2H).

Step 2: Synthesis of 4-cyclopropyl-1-methyl-5-phenyl-1H-pyrazole-3-carboxylic acid (INT 65): 2 M aqueous LiOH (80 μL, 0.160 mmol, 1.20 eq) was added to a solution of ethyl 4-cyclopropyl-1-methyl-5-phenyl-1H-pyrazole-3-carboxylate (36 mg, 0.133 mmol, 1.00 eq) in MeOH (1 mL) and the reaction was stirred at 40° C. After 2 h, more 2 M aqueous LiOH (100 μL, 0.200 mmol, 1.50 eq) was added and stirring continued for a further 3 h at 40° C., 15 h at rt and then a further 3.5 h at 40° C. The solvent was removed under reduced pressure to give the title compound, which was used in the next step without further purification.

Step 3: Synthesis of 4-cyclopropyl-1-methyl-5-phenyl-N-(2-(trifluoromethyl)pyridin-4-yl)-1H-pyrazole-3-carboxamide (I-204): The title compound was synthesized from INT 65 based on procedures described in Example 8 and isolated by reverse phase HPLC as an off-white solid (20 mg, 0.052 mmol, 40%). (ES, m/z): [M+H]⁺ 387.5, ¹H NMR (400 MHz, DMSO) δ 10.90 (s, 1H), 8.70 (m, 1H), 8.47 (m, 1H), 8.18 (m, 1H), 7.86-7.82 (m, 2H), 7.46 (m, 2H), 7.37 (t, J=7.3 Hz, 1H), 3.80 (s, 3H), 2.11-2.04 (m, 1H), 0.70-0.65 (m, 2H), 0.28-0.25 (m, 2H).

### Example 37—Synthesis of Additional Compounds

An additional compound synthesized using the route described above is provided in Table 16.

TABLE 16

| Compound | | |
|---|---|---|
| No. | Structure | Analytical data |
| I-205 | 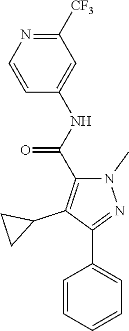 [SMILES: Cn1nc(-c2ccccc2)c(C2CC2)c1C(=O)Nc1ccnc(C(F)(F)F)c1] | (ES, m/z): [M + H]⁺ 387.2, ¹H NMR (400 MHz, DMSO) δ 11.20 (s, 1H), 8.70 (d, J = 5.3 Hz, 1H), 8.25 (d, J = 1.8 Hz, 1H), 7.93 (d, J = 4.3 Hz, 1H), 7.86 − 7.82 (m, 2H), 7.46 (t, J = 7.6 Hz, 2H), 7.37 (t, J = 7.3 Hz, 1H), 3.94 (s, 3H), 2.11 − 2.04 (m, 1H), 0.84 − 0.78 (m, 2H), 0.24 − 0.18 (m, 2H) |

### Example 38—Synthesis of 1-methyl-N-(2-(trifluoromethyl)pyridin-4-yl)-4,5-dihydro-1H-benze[g]indazole-3-carboxamide (1-218)

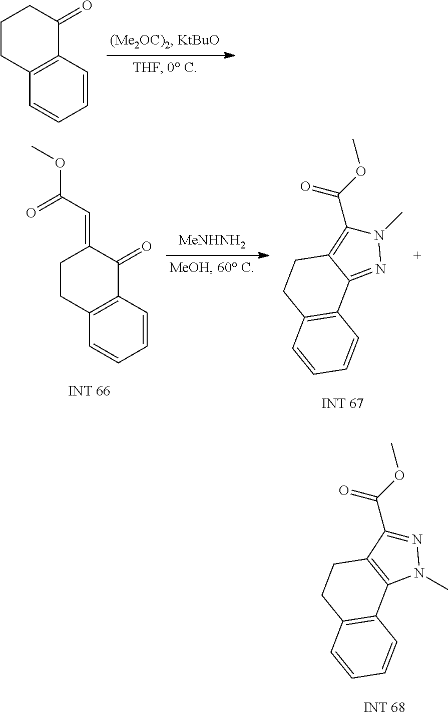

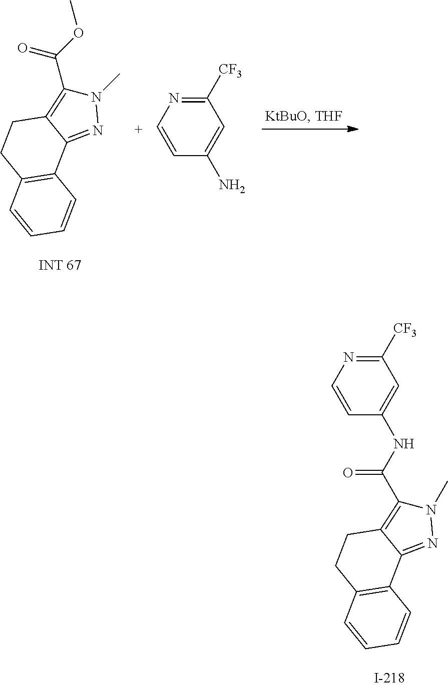 [SMILES: COC(=O)c1c2c(nn1C)-c1ccccc1CC2 ; Nc1ccnc(C(F)(F)F)c1 ; Cn1nc2c(c1C(=O)Nc1ccnc(C(F)(F)F)c1)CCc1ccccc1-2]

Step 1: Synthesis of methyl (E)-2-(1-oxo-3,4-dihydronaphthalen-2 (1H)-ylidene)acetate (INT 66): 1 M KO^{t}Bu in THF (6.4 mL, 6.41 mmol, 1.50 eq) was added dropwise to a solution of 3,4-dihydronaphthalen-1 (2H)-one (0.57 mL, 4.28 mmol, 1.00 eq) and dimethyl oxalate (505 mg, 4.28 mmol, 1.00 eq) in THF (10 mL) under one atmosphere of nitrogen at 0° C. The mixture was warmed to rt and stirred for 3 h, then it was quenched with 1 M aqueous HCl (6.5 mL) and extracted with EtOAc. The combined organic phases were dried (MgSO₄), filtered and concentrated. The crude residue was purified by column chromatography eluting with 0-100% EtOAc in cyclohexane to give the title compound as a yellow oil (802 mg, 3.45 mmol, 81%). ¹H NMR (400 MHz, CDCl₃) δ 8.03-8.00 (m, 1H), 7.52-7.46 (m, 1H), 7.37 (t, J=7.6 Hz, 1H), 7.26 (s, 1H), 7.25 (d, J=6.3 Hz, 1H), 3.93 (s, 3H), 3.00-2.87 (m, 4H).

Step 2: Synthesis of methyl 2-methyl-4,5-dihydro-2H-benzo[g]indazole-3-carboxylate and methyl 1-methyl-4,5-dihydro-1H-benzo[g]indazole-3-carboxylate (INT 67): Methylhydrazine (0.091 mL, 1.72 mmol, 1.00 eq) was added dropwise to a solution of (E)-2-(1-oxo-3,4-dihydronaphthalen-2 (1H)-ylidene)acetate (400 mg, 1.72 mmol, 1.00 eq) in MeOH (10 mL) at rt. Once the addition was complete, the mixture was stirred at 60° C. for 2 h. The reaction mixture was concentrated under reduced pressure. The crude residue was purified by column chromatography eluting with 0-100% EtOAc in cyclohexane to give the two diastereoisomers: Methyl 2-methyl-4,5-dihydro-2H-benzo[g]indazole-3-carboxylate as an orange oil (170 mg, 0.702 mmol, 41%), ¹H NMR (400 MHz, CDCl₃) δ 7.84-7.81 (m, 1H), 7.29-7.21 (m, 3H), 4.21 (s, 3H), 3.92 (s, 3H), 3.04-2.91 (m, 4H). Methyl 1-methyl-4,5-dihydro-2H-benzo[g]indazole-3-carboxylate (INT 68) as a light yellow oil (80 mg, 0.33 mmol, 19%), ¹H NMR (400 MHz, CDCl₃) δ 7.60-7.57 (m, 1H), 7.36-7.24 (m, 3H), 4.24 (s, 3H), 3.94 (s, 3H), 3.21-3.02 (m, 2H), 3.05-2.90 (m, 2H).

Step 3: Synthesis of 2-methyl-N-(2-(trifluoromethyl)pyridin-4-yl)-4,5-dihydro-2H-benzo[g]indazole-3-carboxamide (I-218): The title compound was synthesized from INT 67 based on procedures described in Example 6 and isolated as an off-white solid (61 mg, 0.16 mmol, 47%). (ES, m/z): [M+H]⁺ 373.2, ¹H NMR (400 MHz, DMSO) δ 10.93-10.90 (m, 1H), 8.70 (d, J=5.3 Hz, 1H), 8.22 (d, J=1.5 Hz, 1H), 7.93 (dd, J=2.0, 5.6 Hz, 1H), 7.71 (dd, J=1.4, 7.2 Hz, 1H), 7.32-7.22 (m, 3H), 4.03 (s, 3H), 2.92 (d, J=2.5 Hz, 4H)

### Example 39—Synthesis of Additional Compounds

Additional compounds synthesized using the route described above are provided in Table 17.

TABLE 17

| Compound | | | |
|---|---|---|---|
| No. | Structure | Reagents | Analytical data |
| I-252 | 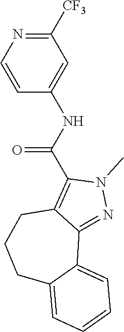 [SMILES: Cn1nc2c(c1C(=O)Nc1ccnc(C(F)(F)F)c1)CCCc1ccccc1-2] | methyl 2-methyl- 2,4,5,6- tetrahydrobenzo [6,7]cyclohepta [1,2-c]pyrazole- 3-carboxylate and 4- (trifluoromethyl) pyridin-2-amine | (ES, m/z): [M + H]⁺ 387.4 ¹H NMR (400 MHz, DMSO) δ 11.09 (d, J = 1.5 Hz, 1H), 8.68 − 8.64 (m, 1H), 8.20 (d, J = 1.8 Hz, 1H), 7.94 (d, J = 7.3 Hz, 1H), 7.87 (d, J = 4.5 Hz, 1H), 7.27 − 7.21 (m, 3H), 4.00 (s, 3H), 2.89 (t, J = 6.4 Hz, 2H), 2.82 − 2.79 (m, 2H), 2.03 − 1.95 (m, 2H) |
| I-253 | 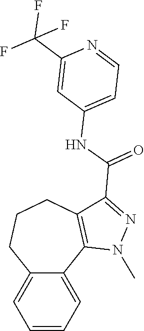 [SMILES: Cn1nc(C(=O)Nc2ccnc(C(F)(F)F)c2)c2c1-c1ccccc1CCC2] | 6,7,8,9- tetrahydro-5H- benzo[7]annulen- 5-one and 4- (trifluoromethyl) pyridin-2-amine | ES, m/z): [M + H]⁺ 387.5 ¹H NMR (400 MHz, DMSO) δ 10.91 (s, 1H), 8.64 (d, J = 5.5 Hz, 1H), 8.47 (d, J = 1.9 Hz, 1H), 8.15 (dd, J = 1.9, 5.5 Hz, 1H), 7.63 − 7.60 (m, 1H), 7.47 − 7.37 (m, 3H), 4.05 (s, 3H), 2.79 − 2.73 (m, 2H), 2.56 − 2.52 (m, 2H), 2.14 (tt, J = 6.9, 6.8 Hz, 2H) |
| I-254 | 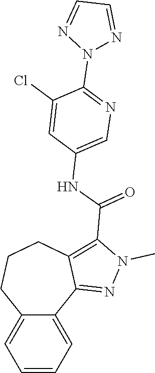 [SMILES: Cn1nc2c(c1C(=O)Nc1cnc(-n3nccn3)c(Cl)c1)CCCc1ccccc1-2] | 6,7,8,9- tetrahydro-5H- benzo[7]annulen- 5-one and 5- chloro-6-(2H- 1,2,3-triazol-2- yl)pyridin-3- amine | (ES, m/z): [M + H]⁺ 420.3, ¹H NMR (400 MHz, DMSO) δ 11.07 (s, 1H), 8.83 (d, J = 2.0 Hz, 1H), 8.63 (d, J = 2.3 Hz, 1H), 8.18 (s, 2H), 7.95 (d, J = 7.3 Hz, 1H), 7.28 − 7.22 (m, 3H), 4.02 (s, 3H), 2.92 (t, J = 6.8 Hz, 2H), 2.84 − 2.79 (m, 2H), 2.05 − 1.98 (m, 2H) |
| I-255 | 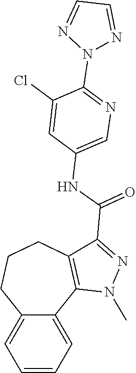 [SMILES: Cn1nc(C(=O)Nc2cnc(-n3nccn3)c(Cl)c2)c2c1-c1ccccc1CCC2] | 6,7,8,9- tetrahydro-5H- benzo[7]annulen- 5-one and 5- chloro-6-(2H- 1,2,3-triazol-2- yl)pyridin-3- amine | ES, m/z): [M + H]⁺ 420.5, ¹H NMR (400 MHz, DMSO) δ 10.89 (s, 1H), 9.06 (d, J = 2.3 Hz, 1H), 8.78 (d, J = 2.3 Hz, 1H), 8.18 (s, 2H), 7.63 − 7.61 (m, 1H), 7.47 − 7.38 (m, 3H), 4.07 (s, 3H), 2.78 (t, J = 7.3 Hz, 2H), 2.55 (t, J = 6.6 Hz, 2H), 2.15 (tt, J = 7.0, 7.0 Hz, 2H) |
| I-256 | 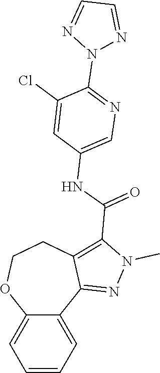 [SMILES: Cn1nc2c(c1C(=O)Nc1cnc(-n3nccn3)c(Cl)c1)CCOc1ccccc1-2] | 3,4- dihydrobenzo[b] oxepin-5(2H)-one and 5-chloro-6- (2H-1,2,3-triazol- 2-yl)pyridin-3- amine | (ES, m/z): [M + H]⁺ 422.3, ¹H NMR (400 MHz, DMSO) δ 11.02 (s, 1H), 8.85 (d, J = 2.0 Hz, 1H), 8.64 (d, J = 2.3 Hz, 1H), 8.24 (dd, J-1.7, 7.9 Hz, 1H), 8.20 (s, 2H), 7.27 − 7.22 (m, 1H), 7.15 − 7.10 (m, 1H), 7.05 (dd, J = 1.2, 8.1 Hz, 1H), 4.29 (t, J = 5.1 Hz, 2H), 4.07 (s, 3H), 3.28 (t, J = 4.9 Hz, 2H) |
| I-257 | 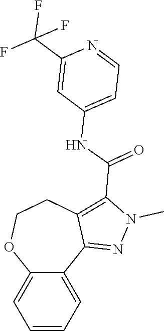 | 3,4- dihydrobenzo[b] oxepin-5(2H)-one and 4- (trifluoromethyl) pyridin-2-amine | (ES, m/z): [M + H]⁺ 398.4., ¹H NMR (400 MHz, DMSO) δ 10.97 (s, 1H), 8.63 (d, J = 5.6 Hz, 1H), 8.15 (d, J = 1.3 Hz., 1H), 8.13 (d, J = 1.7 H7, 1H), 7.85 (dd, J = 1.8, 5.6 Hz, 1H), 7.18 − 7.13 (m, 1H), 7.06 − 7.01 (m, 1H), 6.95 (dd, J = 1.0, 8.2 Hz, 1H), 4.18 (t, J = 5.0 Hz, 2H), 3.96 (s, 3H), 3.15 (t, J = 5.1 Hz, 2H) |
| I-259 | 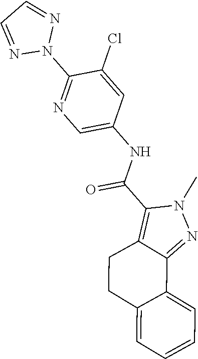 [SMILES: Cn1nc2c(c1C(=O)Nc1cnc(-n3nccn3)c(Cl)c1)CCc1ccccc1-2] | methyl 2-methyl- 4,5-dihydro-2H- benzo[g]indazolc- 3-carboxylate and 5-chloro-6-(2H- 1,2,3-triazol-2- yl)pyridin-3- amine | (ES, m/z): [M + H]⁺ 406.2, ¹H NMR (400 MHz, DMSO) δ 10.88 − 10.85 (m, 1H), 8.85 (d, J = 2.3 Hz, 1H), 8.62 (d, J = 2.3 Hz, 1H), 8.19 − 8.18 (m, 2H), 7.72 (dd, J = 1.3, 7.3 Hz, 1H), 7.33 − 7.24 (m, 3H), 4.05 − 4.05 (m, 3H), 2.96 − 2.94 (m, 4H) |
| I-260 | 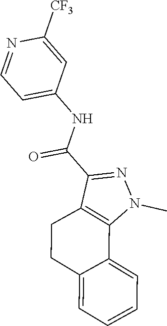 [SMILES: Cn1nc(C(=O)Nc2ccnc(C(F)(F)F)c2)c2c1-c1ccccc1CC2] | methyl 1-methyl- 4,5-dihydro-1H- benzo[g]indazole- 3-carboxylate and 2- (trifluoromethyl) pyridin-4-amine | (ES, m/z): [M + H]⁺ 373.2, ¹H NMR (400 MHz, DMSO) δ 10.89 − 10.86 (m, 1H), 8.63 (d, J = 5.6 Hz, 1H), 8.45 (d, J = 2.0 Hz, 1H), 8.18 − 8.13 (m, 1H), 7.79 (d, J = 7.3 Hz, 1H), 7.41 (t, J = 6.7 Hz, 2H), 7.38 − 7.31 (m, 1H), 4.27 (s, 3H), 3.01 − 2.95 (m, 2H), 2.93 − 2.87 (m, 2H). |

### Example 40—Synthesis of 3-(benzo[c]isothiazol-4-yl)-1-methyl-4-(trifluoromethyl)-N-(2-(trifluoromethyl)pyridin-4-yl)-1H-pyrazole-5-carboxamide (I-210)

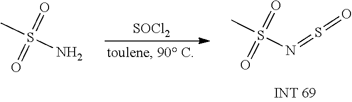

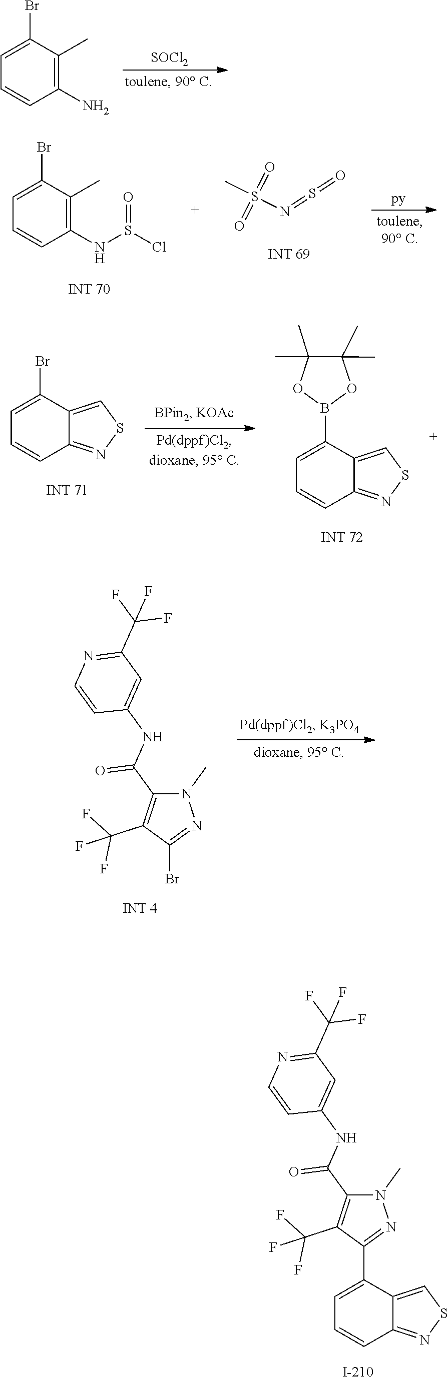

Step 1: Synthesis of N-(oxo-14-sulfaneylidene) methanesulfonamide (INT 69): A mixture of methanesulfonamide (0.76 g, 8.00 mmol, 1.00 eq) and thionyl chloride (1.0 mL, 14.0 mmol, 1.75 eq) in toluene (3 mL) was stirred at 90° C. under one atmosphere of nitrogen for 20 h. Once cooled to rt, the reaction was concentrated under reduced pressure to give crude N-(oxo-14-sulfaneylidene) methanesulfonamide, which was used directly in the next step assuming quantitative yield.

Step 2: Synthesis of (3-bromo-2-methylphenyl)sulfuramidous chloride (INT 70): Thionyl chloride (0.43 mL, 5.91 mmol, 1.10 eq) was added dropwise to a solution of 3-bromo-2-methylaniline (1.00 g, 5.37 mmol, 1.00 eq) in toluene (10 mL) at 0° C., the resulting mixture was stirred at 90° C. under one atmosphere of nitrogen for 20 h. Once cooled to rt, the reaction was concentrated under reduced pressure to give crude (3-bromo-2-methylphenyl) sulfuramidous chloride, which was used directly in the next step assuming quantitative yield.

Step 3: Synthesis of 4-bromobenzo[c]isothiazole (INT 71): A solution of crude N-(oxo-14-sulfaneylidene) methanesulfonamide INT 69 (1138 mg, 8.06 mmol, 1.28 eq) in toluene (2 mL) was added dropwise to a solution of crude (3-bromo-2-methylphenyl) sulfuramidous chloride INT 70 (1.69 g, 6.30 mmol, 1.00 eq) and pyridine (1.0 mL, 12.6 mmol, 2.00 eq) in toluene (4 mL) at 0° C. The resulting mixture was stirred at 90° C. overnight. Once cooled to rt, the reaction was concentrated under reduced pressure. The crude residue was purified by column chromatography eluting with 0-100% EtOAc in cyclohexane, and by preparative HPLC to give 4-bromo-2,1-benzothiazole (230 mg, 1.07 mmol, 17%) as a colourless oil. ¹H NMR (400 MHz, CDCl₃) δ 9.31 (d, J=1.0 Hz, 1H), 7.82-7.79 (m, 1H), 7.46-7.43 (m, 1H), 7.31 (t, J=8.5 Hz, 1H).

Step 4: Synthesis of 4-(4,4,5,5-tetramethyl-1,3,2-dioxaborolan-2-yl)benzo[c]isothiazole (INT 72): The title compound was synthesized based on procedures described in Example 2 and isolated as a yellow oil (147 mg, 0.563 mmol, quant). (ES, m/z): [M+H]⁺ 472.2, ¹H NMR (400 MHz, CDCl₃) δ 9.79-9.78 (m, 1H), 7.97-7.94 (m, 1H), 7.82-7.79 (m, 1H), 7.45 (dd, J=6.5, 8.8 Hz, 1H), 1.28 (s, 12H).

Step 5: Synthesis of 3-(benzo[c]isothiazol-4-yl)-1-methyl-4-(trifluoromethyl)-N-(2-(trifluoromethyl)pyridin-4-yl)-1H-pyrazole-5-carboxamide (I-210): It was synthesized following on procedures described in Example 2 and isolated as an off-white solid (20.49 mg, 0.0427 mmol, 29%). (ES, m/z): [M+H]⁺ 472.2, ¹H NMR (400 MHz, DMSO) δ 11.98 (s, 1H), 9.80 (d, J=1.0 Hz, 1H), 8.76 (d, J=5.1 Hz, 1H), 8.20 (d, J=1.3 Hz, 1H), 7.95-7.89 (m, 2H), 7.64 (dd, J=6.7, 9.0 Hz, 1H), 7.41 (d, J=6.8 Hz, 1H), 4.08 (s, 3H).

### Example 41—Synthesis of 3-(3-bromo-4-chlorophenoxy)oxetane—INT 73

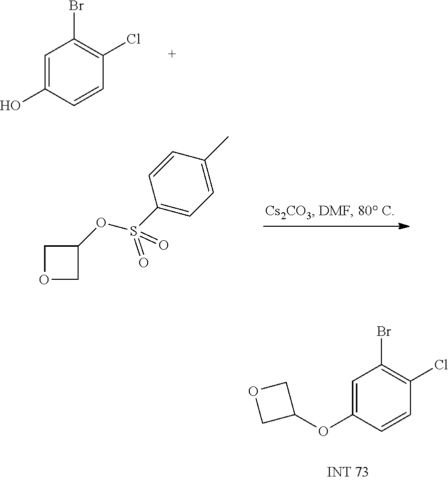

A solution of 3-bromo-4-chloro-phenol (500 mg, 2.41 mmol, 1.00 eq), Cs₂CO₃ (1571 mg, 4.82 mmol, 2.00 eq), oxetan-3-yl 4-methylbenzenesulfonate (825 mg, 3.62 mmol, 1.50 eq), and KI (40 mg, 0.241 mmol, 0.100 eq) in DMF (5 mL) was stirred at 80° C. overnight. The reaction mixture was cooled to rt, poured into water and extracted with EtOAc. The organic layers were dried (MgSO₄) and concentrated under reduced pressure. The crude residue was purified by column chromatography eluting with 0-50% EtOAc in cyclohexane to give the title compound as an off-white solid (500 mg, 1.89 mmol, 79%). ¹H NMR (400 MHz, DMSO) δ 7.51 (d, J=8.8 Hz, 1H), 7.19 (d, J=2.9 Hz, 1H), 6.86 (dd, J=2.9, 8.8 Hz, 1H), 5.34-5.27 (m, 1H), 4.90 (dd, J=6.7, 6.7 Hz, 2H), 4.51 (dd, J=5.0, 7.6 Hz, 2H).

### Example 42—Synthesis of tert-butyl 4-(5-(ethoxycarbonyl)-1-methyl-4-(trifluoromethyl)-1H-pyrazol-3-yl) piperidine-1-carboxylate—INT 74

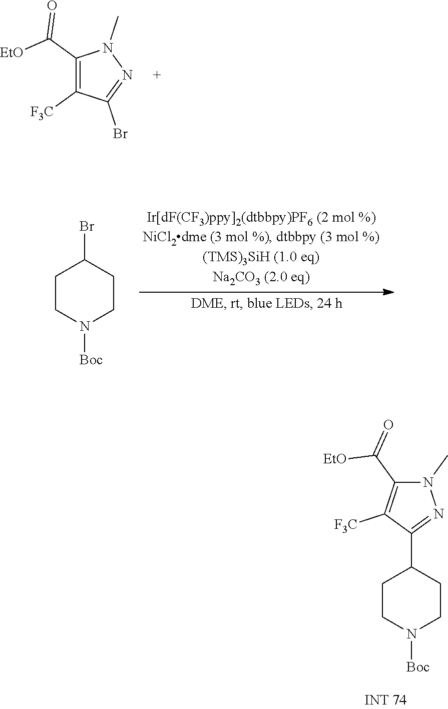 [SMILES: CCOC(=O)c1c(C(F)(F)F)c(Br)nn1C ; CC(C)(C)OC(=O)N1CCC(Br)CC1 ; CCOC(=O)c1c(C(F)(F)F)c(C2CCN(C(=O)OC(C)(C)C)CC2)nn1C]

To a reaction vessel charged with a stirring bar were added (4,4′-di-tert-butyl-2,2′-bipyridine)bis[3,5-difluoro-2-[5-trifluoromethyl-2-pyridinyl-kN)phenyl-kC]iridium (III) hexafluorophosphate (7.5 mg, 6.64 μmol, 0.020 eq), 1-Boc-4-bromopiperidine (131 μL, 0.664 mmol, 2.00 eq), ethyl 5-bromo-2-methyl-4-(trifluoromethyl) pyrazole-3-carboxylate (100 mg, 0.332 mmol, 1.00 eq), NaHCO₃ (56 mg, 0.664 mmol, 2.00 eq) and tris(trimethylsilyl) silane (102 mL, 0.332 mmol, 1.00 eq), and the vessel was purged with nitrogen for 5 minutes before adding ethylene glycol dimethyl ether (1.0 mL). In a separate vial, dtbbpy (27 mg) and NiCl₂·dme complex (22 mg) were charged and the vial was sealed and purged with nitrogen, before 5 mL of DME was added. The suspension was sonicated for 5 minutes, then 500 μL of solution of 4,4′-di-tert-butyl-2,2′-dipyridyl (2.7 mg, 9.96 μmol, 0.030 eq) and nickel (II) chloride ethylene glycol dimethyl ether complex (2.2 mg, 9.96 μmol, 0.030 eq)) was added to the first reaction vessel, which was sealed and stirred 5 cm away from a blue LED light for 18 h. The reaction mixture was diluted with EtOAc and filtered through Celite®. Solvent was removed under reduced pressure and the crude purified by column chromatography with 0-20% EtOAc in cyclohexane to give the title compound (50 mg, 0.123 mmol, 37%) as a colorless oil. (ES, m/z): [M+H]⁺ 306.3.

### Example 43—Synthesis of 3-(2-bromophenoxy)-oxetane—INT 75

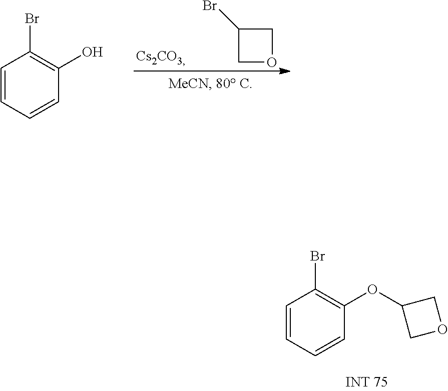 [SMILES: Oc1ccccc1Br ; BrC1COC1 ; Brc1ccccc1OC1COC1]

2-Bromophenol (1.2 mL, 10.0 mmol, 1.00 eq) and 3-bromooxetane (2.5 mL, 30.1 mmol, 3.01 eq) were added to a suspension of Cs₂CO₃ (9.82 g, 30.1 mmol, 3.01 eq) in MeCN (155 mL) and the reaction was stirred at 80° C. for 24 hours. The reaction mixture was cooled down to rt, filtered through Celite® and concentrated under reduced pressure. The crude residue was purified by column chromatography eluting with 0-20% EtOAc in cyclohexane to give the title compound (2,067 mg, 9.02 mmol, 90%) as a colourless oil. LCMS pH 2, rt=1.57 minutes, no molecular ion observed for this compound.

### Example 44—Synthesis of 8-bromo-4-methyl-3,4-dihydro-2H-benzo[b][1,4]oxazine—INT 76

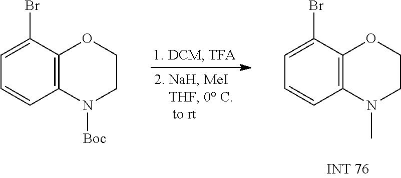 [SMILES: CC(C)(C)OC(=O)N1CCOc2c(Br)cccc21 ; CN1CCOc2c(Br)cccc21]

Step 1. Synthesis of 8-bromo-3,4-dihydro-2H-benzo[b][1,4]oxazine: A solution of tert-butyl 8-bromo-2,3-dihydro-1,4-benzoxazine-4-carboxylate (500 mg, 1.59 mmol, 1.00 eq) in DCM (3 mL) and TFA (1 mL) was stirred at rt for 2 hours. The solvent was removed under reduced pressure and the crude was purified by SCX eluting with 7 N NH₃ in MeOH. The solvent was evaporated under reduced pressure to give the title compound (340 mg, 1.59 mmol, 100%) as a brown oil that was directly used in the next step without further purification. (ES, m/z): [M+H]⁺ 214.2 and 216.2, ¹H NMR (400 MHz, CDCl₃) δ 6.88 (dd, J=7.9, 1.3 Hz, 1H), 6.62 (t, J=7.9 Hz, 1H), 6.52 (dd, J=7.9, 1.4 Hz, 1H), 4.35 (t, J=4.4 Hz, 2H), 3.44 (t, J=4.4 Hz, 2H).

Step 2. Synthesis of 8-bromo-4-methyl-3,4-dihydro-2H-benzo[b][1,4]oxazine (INT 76): A suspension of NaH (60%, 31 mg, 0.785 mmol, 1.20 eq) in THF (1 mL) was added to a stirred solution of 8-bromo-3,4-dihydro-2H-1,4-benzoxazine (140 mg, 0.654 mmol, 1.00 eq) in THF (2 mL) at 0° C. under a nitrogen atmosphere and the resulting mixture was stirred for 40 minutes. MeI (45 mL, 0.719 mmol, 1.10 eq) was added and the reaction was allowed to warm up to rt. After 2 hours, more MeI (20 mL, 0.327 mmol, 0.50 eq) was added and the reaction was stirred at rt for 40 hours. The mixture was quenched with saturated aqueous NH₄Cl and diluted with EtOAc. The aqueous layer was separated and extracted with EtOAc. The combined organic phases were washed with brine, dried (MgSO₄), filtered and the concentrated under reduced pressure. The crude residue was purified by column chromatography eluting with 0-10% EtOAc in cyclohexane to give the title compound (118 mg, 0.517 mmol, 79%) as a yellow oil. (ES, m/z): [M+H]⁺ 228.2 and 230.2, ¹H NMR (400 MHz, CDCl₃) δ 6.88 (dd, J=8.0, 1.4 Hz, 1H), 6.70 (t, J=8.0 Hz, 1H), 6.59 (dd, J=8.1, 1.4 Hz, 1H), 4.39 (t, J=4.5 Hz, 2H), 3.30 (t, J=4.5 Hz, 2H), 2.90 (s, 3H).

### Example 45—Synthesis of 4-bromo-1-methyl-indole—INT 77

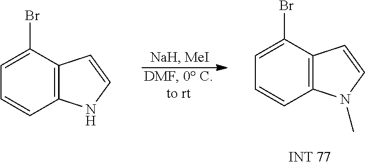

4-Bromoindole (1000 mg, 5.10 mmol, 1.00 eq) was added dropwise to a stirred suspension of NaH (60%, 306 mg, 7.65 mmol, 1.50 eq) in DMF (5 mL) at 0° C. under a nitrogen atmosphere and the resulting mixture was stirred for 90 minutes. MeI (0.48 mL, 7.65 mmol, 1.50 eq) was added and the reaction was allowed to warm up to rt and stirred for 30 minutes. The reaction was quenched with water and extracted with Et₂O. The combined organic layers were washed with brine, dried (MgSO₄), filtered and concentrated under reduced pressure. The crude was purified by column chromatography eluting with 0-20% EtOAc in cyclohexane to give the title compound (950 mg, 4.52 mmol, 89%) as a yellow oil. (ES, m/z): [M+H]⁺ 210.2 and 212.2, ¹H NMR (400 MHz, CDCl₃) δ 7.28 (d, J=7.5 Hz, 1H), 7.26 (d, J=8.3 Hz, 1H), 7.10 (d, J=3.1 Hz, 1H), 7.06 (d, J=7.9 Hz, 1H), 6.53 (d, J=3.1 Hz, 1H), 3.79 (s, 3H).

### Example 46—Synthesis of 2-(4-bromoindol-1-yl)acetate—INT 78

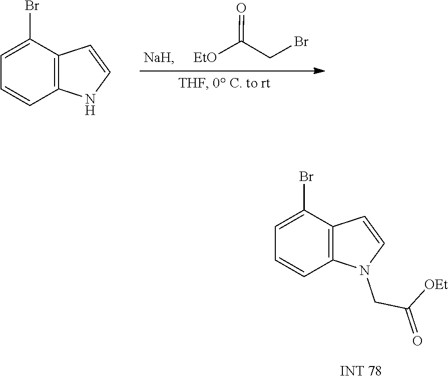 [SMILES: Brc1cccc2[nH]ccc12 ; CCOC(=O)CBr ; CCOC(=O)Cn1ccc2c(Br)cccc21]

4-Bromoindole (0.64 mL, 5.10 mmol, 1.00 eq) was added dropwise to a stirred suspension of NaH (60%, 306 mg, 7.65 mmol, 1.50 eq) in DMF (5 mL) at 0° C. under a nitrogen atmosphere, the resulting mixture was stirred for 90 minutes. Then, ethyl bromoacetate (0.85 mL, 7.65 mmol, 1.50 eq) was added and the reaction was allowed to warm up to rt and stirred for 30 minutes. The reaction was quenched with water (20 mL) and extracted with Et₂O. The combined organic phases were washed with brine, dried (MgSO₄), filtered and the concentrated under reduced pressure. The crude residue was purified by column chromatography eluting with 0-50% EtOAc in cyclohexane to give the title compound (1,025 mg, 3.63 mmol, 71%) as a yellow oil. (ES, m/z): [M+H]⁺ 282.2 and 284.2, ¹H NMR (400 MHz, CDCl₃) δ 7.30 (d, J=7.5 Hz, 1H), 7.20 (d, J=8.2 Hz, 1H), 7.14 (d, J=3.2 Hz, 1H), 7.08 (t, J=7.9 Hz, 1H), 6.61 (dd, J=3.2, 0.5 Hz, 1H), 4.82 (s, 2H), 4.21 (q, J=7.1 Hz, 2H), 1.25 (t, J=7.1 Hz, 3H). By-product (2-ethoxy-2-oxo-ethyl) 2-(4-bromoindol-1-yl)acetate—INT 79 (500 mg, 1.47 mmol, 29%), arising from partial hydrolysis of the target material and reaction with the excess ethyl bromoacetate, was also isolated by column chromatography eluting with 20% EtOAc in cyclohexane. (ES, m/z): [M+H]⁺ 340.2 and 342.2, ¹H NMR (400 MHz, CDCl₃) δ 7.31 (d, J=7.6 Hz, 1H), 7.25 (d, J=8.3 Hz, 1H), 7.16 (d, J=3.3 Hz, 1H), 7.09 (t, J=7.9 Hz, 1H), 6.62 (dd, J=3.3, 0.5 Hz, 1H), 4.97 (s, 2H), 4.66 (s, 2H), 4.21 (q, J=7.1 Hz, 2H), 1.26 (t, J=7.1 Hz, 3H).

### Example 47—Synthesis of 2-(4-bromoindol-1-yl)EtOH—INT 80

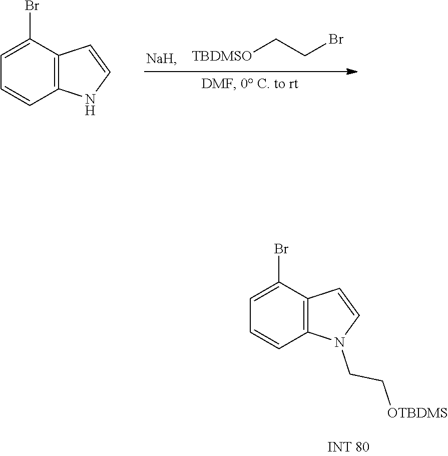 [SMILES: Brc1cccc2[nH]ccc12 ; CC(C)(C)[Si](C)(C)OCCBr ; CC(C)(C)[Si](C)(C)OCCn1ccc2c(Br)cccc21]

4-Bromoindole (0.64 mL, 5.10 mmol, 1.00 eq) was added dropwise to a stirred suspension of NaH (60%, 306 mg, 7.65 mmol, 1.50 eq) in DMF (5 mL) at 0° C. under a nitrogen atmosphere, the resulting mixture was stirred for 90 minutes. Then, (2-bromoethoxy)-tert-butyldimethylsilane (1.6 mL, 7.65 mmol, 1.50 eq) was added and the mixture was allowed to warm to rt and stirred for 24 hours. The reaction was quenched with water and extracted with Et₂O. The combined organics were washed with brine, dried (MgSO₄), filtered and the solvent was removed under reduced pressure. The crude was purified by column chromatography eluting with 0-20% EtOAc in cyclohexane to give the title compound (419 mg, 1.75 mmol, 34%) as an orange oil. (ES, m/z): [M+H]⁺ 240.2 and 242.2, ¹H NMR (400 MHz, CDCl₃) δ 7.32 (d, J=8.2 Hz, 1H), 7.29 (d, J=7.5 Hz, 1H), 7.22 (d, J=3.2 Hz, 1H), 7.07 (t, J=7.9 Hz, 1H), 6.58 (d, J=3.2 Hz, 1H), 4.28 (t, J=5.2 Hz, 2H), 3.96 (q, J=5.4 Hz, 2H), 1.49 (t, J=5.9 Hz, 1H).

### Example 48-Synthesis of tert-butyl N-(4-bromo-1-isoquinolyl)-N-tert-butoxycarbonyl-carbamate—INT 81

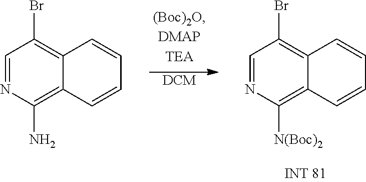

Et₃N (1.3 mL, 9.41 mmol, 2.10 eq) and DMAP (548 mg, 4.48 mmol, 1.00 eq) were added to a solution of 4-bromoisoquinolin-1-amine (1.0 g, 4.48 mmol, 1.00 eq) and Boc₂O (2.1 g, 9.41 mmol, 2.10 eq) in DCM (25 mL) and the reaction was stirred at rt for 18 hours. The solvent was removed under reduced pressure and the crude residue was purified by column chromatography eluting with 0-10% EtOAc in cyclohexane to give the title compound (1,627 mg, 3.84 mmol, 86%) as a light yellow solid. (ES, m/z): [M+H]⁺ 423.4 and 425.4, ¹H NMR (400 MHz, CDCl₃) δ 8.63 (s, 1H), 8.20 (d, J=8.4 Hz, 1H), 7.96 (d, J=8.4 Hz, 1H), 7.84 (dt, J=7.7, 0.9 Hz, 1H), 7.70 (t, J=7.7 Hz, 1H), 1.32 (s, 18H).

### Example 49—Synthesis of tert-butyl N-(5-bromo-1-isoquinolyl)-N-tert-butoxycarbonyl-carbamate—INT 82

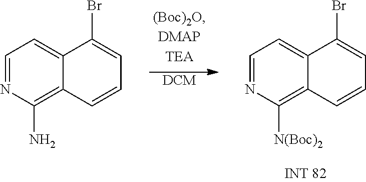

Et₃N (0.9 mL, 6.72 mmol, 3.00 eq) and DMAP (55 mg, 0.448 mmol, 0.200 eq) were added to a solution of 5-bromoisoquinolin-1-amine (500 mg, 2.24 mmol, 1.00 eq) and Boc₂O (1.3 g, 6.72 mmol, 3.00 eq) in MeCN (5 mL) and the reactions was stirred at rt for 18 hours. The solvent was removed under reduced pressure and the crude residue was purified by column chromatography eluting with 0-20% EtOAc in cyclohexane to give the title compound (693 mg, 1.64 mmol, 73%) as a light yellow solid. (ES, m/z): [M+H]⁺ 423.4 and 425.4, ¹H NMR (400 MHz, CDCl₃) δ 8.53 (d, J=6.0 Hz, 1H), 8.02 (dd, J=6.0, 0.6 Hz, 1H), 7.99 (dd, J=7.5, 0.9 Hz, 1H), 7.95 (d, J=8.3 Hz, 1H), 7.48 (dd, J=8.3, 7.5 Hz, 1H), 1.31 (s, 18H).

### Example 50—Synthesis of 8-bromo-4-methyl-1,4-benzoxazin-3-one—INT 83

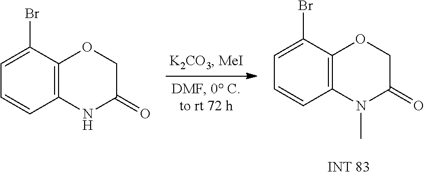

MeI (33 mL, 0.526 mmol, 1.50 eq) was added to stirred mixture of 8-bromo-4H-1,4-benzoxazin-3-one (80 mg, 0.351 mmol, 1.00 eq) and K₂CO₃ (145 mg, 1.05 mmol, 3.00 eq) in DMF (5 mL) and stirred at rt for 72 hours. The mixture was diluted with EtOAc (50 mL) and a 1:1 water/brine mixture (30 mL). The phases were separated and the organic layer was further washed with water, brine, then dried (MgSO₄), filtered and the solvent was removed under reduced pressure. The crude was purified by column chromatography eluting with 0-20% EtOAc in cyclohexane to give the title compound (65 mg, 0.269 mmol, 77%) as an orange solid. (ES, m/z): [M+H]⁺ 242.2 and 244.2 ¹H NMR (400 MHz, CDCl₃) δ 7.28-7.22 (m, 1H), 6.93 (d, J=4.7 Hz, 2H), 4.72 (s, 2H), 3.36 (s, 3H).

### Example 51—Synthesis of 7-bromo-5-methoxy-1,3-benzothiazole—INT 87

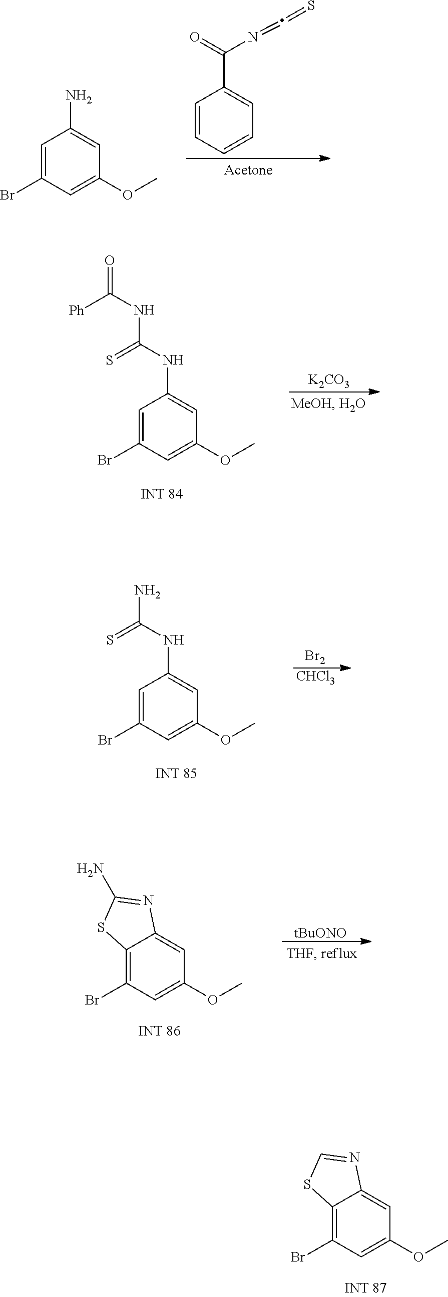

Step 1: Synthesis of N-((3-bromo-5-methoxyphenyl)carbamothioyl)benzamide (INT 84): Benzoyl isothiocyanate (1.6 mL, 11.9 mmol, 1.20 eq) was added to a stirred solution of 3-bromo-5-methoxy-aniline (2,000 mg, 9.90 mmol, 1.00 eq) in acetone (40 mL) the mixture was stirred for 2 hours at rt. The solvent was removed under reduced pressure and the residue was triturated with cyclohexane, filtered and the solid was washed with cyclohexane and dried. The filtrate was evaporated under reduced pressure and the resulting solid was triturated in cyclohexane and filtered. The two solid batches were combined and dried to give the title compound (3.6 g, 9.86 mmol, 100%) as an off-white solid. (ES, m/z): [M+H]⁺ 364.9 and 366.9 ¹H NMR (400 MHz, DMSO) δ 12.61 (br s, 1H), 11.68 (s, 1H), 8.03 (d, J=7.2 Hz, 2H), 7.72 (t, J=7.4 Hz, 1H), 7.65 (s, 1H), 7.60 (t, J=7.7 Hz, 2H), 7.40 (t, J=1.9 Hz, 1H), 7.12 (t, J=1.9 Hz, 1H), 3.85 (s, 3H).

Step 2: Synthesis of 1-(3-bromo-5-methoxyphenyl)thiourea (INT 85): K₂CO₃ (1956 mg, 14.2 mmol, 2.80 eq) was added to a mixture of N-[(3-bromo-5-methoxy-phenyl)carbamothioyl]benzamide (1,846 mg, 5.05 mmol, 1.00 eq) in MeOH (25 mL) and water (10 mL). The resulting reaction mixture was stirred at 70° C. for 16 hours. After cooling down to rt, the solvent was removed under reduced pressure. The residue was suspended in water (10 mL) and the solid was filtered, washed with ice-cold water and dried to give the title compound (922 mg, 3.53 mmol, 70%) as a grey solid. (ES, m/z): [M+H]⁺ 260.9 and 262.9, ¹H NMR (400 MHz, DMSO) δ 9.83 (s, 1H), 8.24-7.23 (br s, 2H), 7.35 (s, 1H), 7.13 (s, 1H), 6.93 (s, 1H), 3.80 (s, 3H).

Step 3: Synthesis 7-bromo-5-methoxybenzo[d]thiazol-2-amine (INT 86): A solution of bromine (0.18 mL, 3.53 mmol, 1.00 eq) in chloroform (8 mL) was added dropwise to a stirred solution of (3-bromo-5-methoxy-phenyl)thiourea (922 mg, 3.53 mmol, 1.00 eq) in chloroform (30 mL) at −78° C. under a nitrogen atmosphere. The resulting mixture was allowed to warm up to rt and stirred for 15 minutes, then it was stirred at 70° C. for 1 hour. The reaction was allowed to cool down to rt and basified with 28% aqueous NH₃ solution. The phases were separated and the aqueous one was extracted with EtOAc (3×20 mL). The combined organic phases were washed with brine, dried (MgSO₄), filtered and the solvent was evaporated to give the title compound (800 mg, 3.09 mmol, 87%) as an off-white solid. (ES, m/z): [M+H]⁺ 258.9 and 260.9, ¹H NMR (400 MHz, CDCl₃) δ 7.70 (s, 2H), 6.90 (d, J=2.2 Hz, 1H), 6.84 (d, J=2.2 Hz, 1H), 3.76 (s, 3H).

Step 4: Synthesis of 7-bromo-5-methoxy-1,3-benzothiazole (INT 87): tert-Butyl nitrite (90%, 2.8 mL, 21.3 mmol, 6.50 eq) was added to a solution of 7-bromo-5-methoxy-1,3-benzothiazol-2-amine (850 mg, 3.28 mmol, 1.00 eq) in THF (20 mL) was added and the resulting reaction mixture was stirred at 50° C. for 3 hours, then allowed to cool down to rt and concentrated under reduced pressure. The crude residue was purified by column chromatography eluting with 0-50% EtOAc in cyclohexane to give the title compound (794 mg, 3.25 mmol, 99%) as a yellow solid. (ES, m/z): [M+H]⁺ 243.9 and 245.9, ¹H NMR (400 MHz, CDCl₃) δ 9.02 (s, 1H), 7.56 (d, J=2.2 Hz, 1H), 7.27 (d, J=2.2 Hz, 1H), 3.90 (s, 3H).

### Example 52—Synthesis of 7-bromo-6-methyl-1,3-benzothiazol-2-amine—INT 91

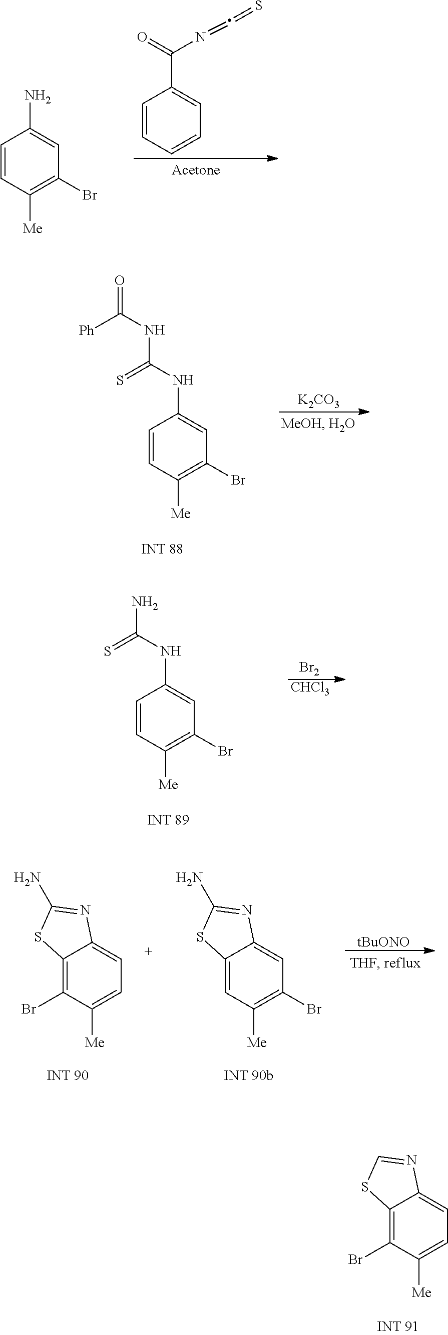

Step 1: Synthesis of N-((3-bromo-4-methylphenyl)carbamothioyl)benzamide (INT 88): Benzoyl isothiocyanate (2.0 mL, 15.2 mmol, 1.20 eq) was added to a solution of 3-bromo-4-methylaniline (2.4 g, 12.6 mmol, 1.00 eq) in acetone (60 mL), the reaction was stirred at 60° C. for 4 hours. After cooling down to rt, the solvent was removed under reduced pressure and the crude solid was triturated with cyclohexane, filtered and dried under reduced pressure to give the title compound (4.3 g, 12.4 mmol, 98%) as a beige solid. (ES, m/z): [M+H]⁺ 348.9 and 350.9, ¹H NMR (400 MHz, DMSO) δ 12.57 (s, 1H), 11.62 (s, 1H), 8.06 (s, 1H), 7.98 (d, J=7.4 Hz, 2H), 7.67 (t, J=7.4 Hz, 1H), 7.59-7.48 (m, 3H), 7.40 (d, J=8.2 Hz, 1H), 2.36 (s, 3H).

Step 2: Synthesis of 1-(3-bromo-4-methylphenyl)thiourea (INT 89): 2.3 M aqueous K₂CO₃ solution (15 mL, 34.6 mmol, 2.80 eq) was added to a solution of N-[(3-bromo-4-methyl-phenyl)carbamothioyl]benzamide (4.3 g, 12.4 mmol, 1.00 eq) in MeOH (30 mL). The resulting reaction mixture was stirred at 70° C. for 2 hours, then allowed to cool down to rt and concentrated under reduced pressure. The residue was diluted with water, acidified to pH 2 with 2 M aqueous HCl solution and the precipitate was filtered, dried and triturated with EtOAc to give the title compound (2,694 mg, 9.34 mmol, 76%) as an off-white solid, containing ca 15% of benzoic acid, which was used in the next step without further purification. (ES, m/z): [M+H]⁺ 244.9 and 246.9, ¹H NMR (400 MHz, DMSO) δ 9.86 (s, 1H), 7.82 (s, 1H), 7.63 (br s, 2H), 7.36-7.20 (m, 2H), 2.35 (s, 3H).

Step 3: Synthesis of 7-bromo-6-methylbenzo[d]thiazol-2-amine (INT 90): Bromine (0.62 mL, 12.1 mmol, 1.10 eq) was added dropwise to a stirred solution of (3-bromo-4-methyl-phenyl)thiourea (2.7 g, 11.0 mmol, 1.00 eq) in acetic acid (50 mL) at 10° C. under a nitrogen atmosphere, The resulting mixture was allowed to warm up to rt and stirred for 15 minutes, then it was stirred at 80° C. for 2 hours. Once cooled to rt, the reaction was poured into water (50 mL) and extracted with EtOAc. The organic phase was washed with brine, dried (MgSO₄), filtered and the solvent was evaporated under reduced pressure. The residue was triturated with 5:1 cyclohexane in EtOAc. The solid was dried to give the title compound (868 mg, 3.57 mmol, 32%) as an off-white solid, which was used in the next step without further purification. The filtrate was purified by column chromatography eluting with 0-50% EtOAc in cyclohexane to give a mixture of two regioisomers. Another trituration with 5:1 cyclohexane:EtOAc gave two mixed fractions: solid (696 mg of a 1:1.15 mixture of regioisomers INT 90:INT 90b) and filtrate (710 mg of a 2:1 mixture of regioisomer INT 90: INT 90b). 7-bromo-6-methyl-1,3-benzothiazol-2-amine—INT 90 (ES, m/z): [M+H]⁺⁰ 242.9 and 244.9, ¹H NMR (400 MHz, DMSO) δ 7.38 (d, J=8.2 Hz, 1H), 7.18 (d, J=8.2 Hz, 1H), 5.11 (br s, 2H), 2.44 (s, 3H). 5-bromo-6-methyl-1,3-benzothiazol-2-amine—INT 90b; ¹H NMR (400 MHz, CDCl₃) δ 7.72 (s, 1H), 7.43 (s, 1H), 5.38 (br s, 2H), 2.43 (s, 3H).

Step 4: Synthesis of 7-bromo-6-methylbenzo[d]thiazole (INT 91): tert-Butyl nitrite (90%, 1.4 mL, 10.7 mmol, 3.00 eq) was added to a solution of 7-bromo-6-methyl-1,3-benzothiazol-2-amine (868 mg, 3.57 mmol, 1.00 eq) in THF (20 mL) and the reaction was stirred at 70° C. for 18 hours. The mixture was allowed to cool down to rt and solvent was removed under reduced pressure. The crude residue was purified by column chromatography eluting with 0-40% EtOAc in cyclohexane to give the title compound (540 mg, 2.37 mmol, 66%) as a yellow solid. (ES, m/z): [M+H]⁺ 227.9 and 229.9, ¹H NMR (400 MHz, CDCl₃) δ 8.96 (s, 1H), 7.97 (d, J=8.3 Hz, 1H), 7.40 (d, J=8.3 Hz, 1H), 2.56 (s, 3H).

### Example 53—Synthesis of Additional Compounds

Table 18 shows boronate compounds that were cither synthesized or obtained from commercial sources.

TABLE 18

| Intermediate No. | Structure | CAS Registry No. or Analytical Data |
|---|---|---|
| INT 92 | 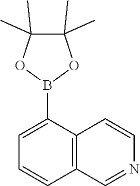 [SMILES: CC1(C)OB(c2cccc3cnccc23)OC1(C)C] | CAS: 1338914-81-0 |
| INT 93 | 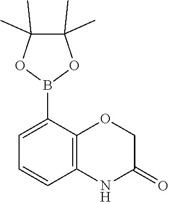 [SMILES: CC1(C)OB(c2cccc3c2OCC(=O)N3)OC1(C)C] | ES− (m/z) = 274.1 [M − H]⁻ ¹H NMR (400 MHz, CDCl₃) d 7.80 (s, 1H), 7.39 (dd, J = 1.6, 7.4 Hz, 1H), 6.96 (dd, J = 7.5, 7.5 Hz, 1H), 6.85 (dd, J = 1.6, 7.7 Hz, 1H), 4.67 (s, 2H), 1.36 (s, 12H) |
| INT 94 | 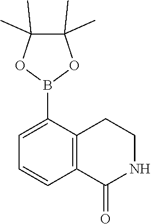 [SMILES: CC1(C)OB(c2cccc3c2CCNC3=O)OC1(C)C] | ES+ (m/z) = 274.2 [M + H]⁺ ¹H NMR (400 MHz, CDCl₃) d 8.18 (dd, J = 1.6, 7.7 Hz, 1H), 7.96 (dd, J = 1.5, 7.4 Hz, 1H), 7.35 (dd, J = 7.6, 7.6 Hz, 1H), 6.01 − 5.98 (m, 1H), 3.54 (dt, J = 2.9, 6.7 Hz, 2H), 3.35 (t, J = 6.7 Hz, 2H), 1.35 (s, 12H). |
| INT 95 | 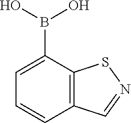 [SMILES: OB(O)c1cccc2cnsc12] | ES+ (m/z) = 179.9 [M + H]⁺ |
| INT 96 | 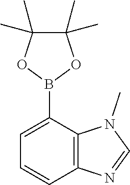 [SMILES: Cn1cnc2cccc(B3OC(C)(C)C(C)(C)O3)c21] | CAS: 2137501-50-7 |
| INT 97 | 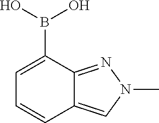 | CAS: 1001907-58-9 |
| INT 98 | 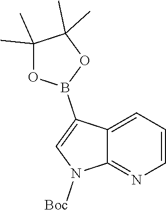 [SMILES: CC(C)(C)OC(=O)n1cc(B2OC(C)(C)C(C)(C)O2)c2cccnc21] | CAS: 942070-47-5 |
| INT 99 | 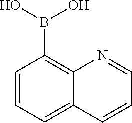 [SMILES: OB(O)c1cccc2cccnc12] | CAS: 86-58-8 |
| INT 100 | 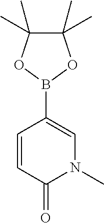 | CAS: 1002309-52-5 |
| INT 101 | 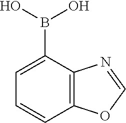 [SMILES: OB(O)c1cccc2ocnc12] | CAS: 2250340-67-9 |
| INT 102 | 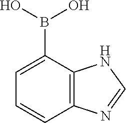 [SMILES: OB(O)c1cccc2nc[nH]c12] | CAS: 499769-95-8 |
| INT 103 | 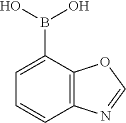 [SMILES: OB(O)c1cccc2ncoc12] | CAS: 1429665-03-1 |
| INT 104 | 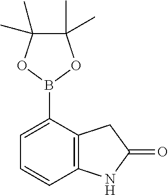 [SMILES: CC1(C)OB(c2cccc3c2CC(=O)N3)OC1(C)C] | CAS: 837392-64-0 |
| INT 105 | 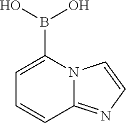 [SMILES: OB(O)c1cccc2nccn12] | CAS: 1428344-34-6 |
| INT 106 | 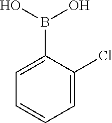 | CAS: 3900-89-8 |
| INT 107 | 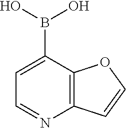 [SMILES: OB(O)c1ccnc2ccoc12] | ES+ (m/z) = 164.1 [M + H]⁺ |
| INT 108 | 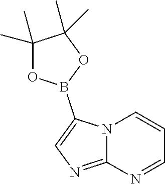 [SMILES: CC1(C)OB(c2cnc3ncccn23)OC1(C)C] | CAS: 1351374-78-1 |
| INT 109 | 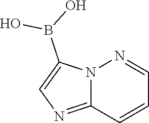 [SMILES: OB(O)c1cnc2cccnn12] | ES+ (m/z) = 164.1 [M + H]⁺ |
| INT 110 | 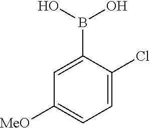 [SMILES: COc1ccc(Cl)c(B(O)O)c1] | CAS: 89694-46-2 |
| INT 111 | 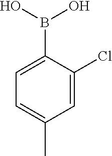 | CAS: 145349-62-8 |
| INT 112 | 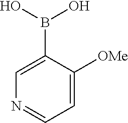 [SMILES: COc1ccncc1B(O)O] | CAS: 355004-67-0 |
| INT 113 | 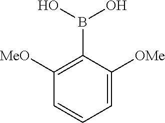 [SMILES: COc1cccc(OC)c1B(O)O] | CAS: 23112-96-1 |
| INT 114 | 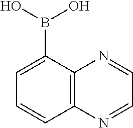 [SMILES: OB(O)c1cccc2nccnc12] | CAS: 1310707-17-5 |
| INT 115 | 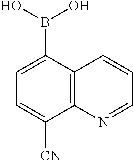 [SMILES: N#Cc1ccc(B(O)O)c2cccnc12] | ES+ (m/z) = 197.3 [M + H]⁺ |
| INT 116 | 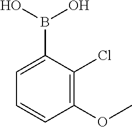 | CAS: 854778-30-6 |
| INT 117 | 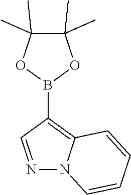 [SMILES: CC1(C)OB(c2cnn3ccccc23)OC1(C)C] | CAS: 1207557-48-9 |
| INT 118 | 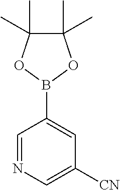 [SMILES: CC1(C)OB(c2cncc(C#N)c2)OC1(C)C] | CAS: 402718-29-0 |
| INT 119 | 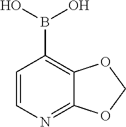 [SMILES: OB(O)c1ccnc2c1OCO2] | ES+ (m/z) =168.1 [M + H]⁺ |
| INT 120 | 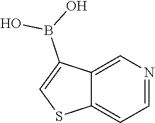 [SMILES: OB(O)c1csc2ccncc12] | CAS: 1909346-59-3 |
| INT 121 | 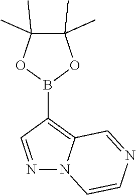 [SMILES: CC1(C)OB(c2cnn3ccncc23)OC1(C)C] | CAS: 1331768-81-0 |
| INT 122 | 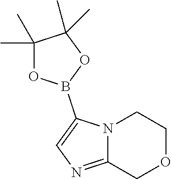 [SMILES: CC1(C)OB(c2cnc3n2CCOC3)OC1(C)C] | CAS: 2246565-04-6 |
| INT 123 | 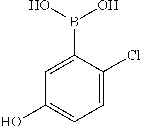 [SMILES: OB(O)c1cc(O)ccc1Cl] | CAS: 913835-71-9 |
| INT 124 | 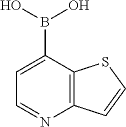 [SMILES: OB(O)c1ccnc2ccsc12] | CAS: 1492031-92-1 |
| INT 125 | 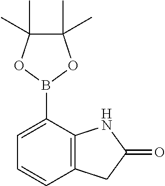 [SMILES: CC1(C)OB(c2cccc3c2NC(=O)C3)OC1(C)C] | CAS: 1150271-45-6 |
| INT 126 | 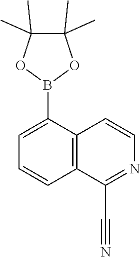 [SMILES: CC1(C)OB(c2cccc3c(C#N)nccc23)OC1(C)C] | ES+ (m/z) = 281.2 [M + H]⁺ ¹H NMR (400 MHz, CDCl₃) d 8.85 (d, J = 6.1 Hz, 1H), 8.68 (d, J = 5.8 Hz, 1H), 8.45 (d, J-8.4 Hz, 1H), 8.39 (d, J = 6.8 Hz, 1H), 7.79 (dd, J = 6.9, 8.4 Hz, 1H), 1.43 (s, 12H). |
| INT 127 | 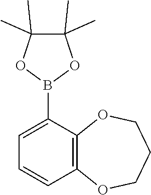 [SMILES: CC1(C)OB(c2cccc3c2OCCCO3)OC1(C)C] | CAS: 2246880-24-8 |
| INT 128 | 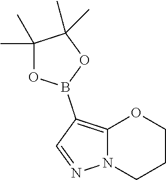 [SMILES: CC1(C)OB(c2cnn3c2OCCC3)OC1(C)C] | CAS: 1383675-83-9 |
| INT 129 | 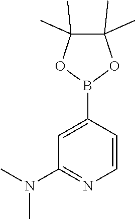 | CAS: 1321518-05-1 |
| INT 130 | 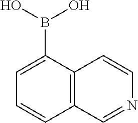 [SMILES: OB(O)c1cccc2cnccc12] | CAS: 371766-08-4 |
| INT 131 | 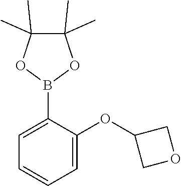 | ES+ (m/z) = 227.0 [M + H]⁺ ¹H NMR (400 MHz, CDCl₃) δ 7.66 (dd, J = 1.8, 7.3 Hz, 1H), 7.33 (dtd, J = 1.8, 7.4, 8.2 Hz, 1H), 6.98 (td, J = 0.8, 7.4 Hz, 1H), 6.41 (d, J = 8.2 Hz, 1H), 5.22 (qd, J = 0.7, 5.4 Hz, 1H), 4.95 (dd, J = 6.8, 7.8 Hz, 1H), 4.83-4.77 (m, 2H), 1.36 (s, 12H) |
| INT 132 | 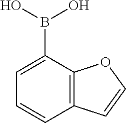 [SMILES: OB(O)c1cccc2ccoc12] | CAS: 1432610-21-3 |
| INT 133 | 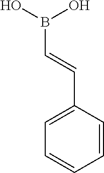 [SMILES: OB(O)/C=C/c1ccccc1] | CAS: 6783-05-7 |
| INT 134 | 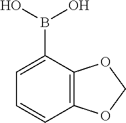 [SMILES: OB(O)c1cccc2c1OCO2] | CAS: 361456-68-0 |
| INT 135 | 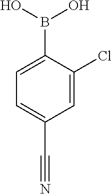 [SMILES: N#Cc1ccc(B(O)O)c(Cl)c1] | CAS: 677743-50-9 |
| INT 136 | 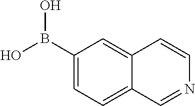 [SMILES: OB(O)c1ccc2cnccc2c1] | CAS: 899438-92-7 |
| INT 137 | 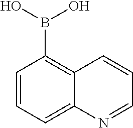 [SMILES: OB(O)c1cccc2ncccc12] | CAS: 355386-94-6 |
| INT 138 | 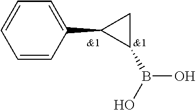 [SMILES: OB(O)[C@H]1C[C@@H]1c1ccccc1] | CAS: 204378-97-2 |
| INT 139 | 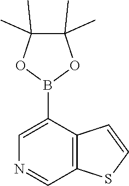 [SMILES: CC1(C)OB(c2cncc3sccc23)OC1(C)C] | CAS: 2230209-53-5 |
| INT 140 | 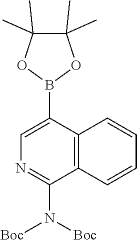 [SMILES: CC(C)(C)OC(=O)N(C(=O)OC(C)(C)C)c1ncc(B2OC(C)(C)C(C)(C)O2)c2ccccc12] | ES+ (m/z) = 471.6 [M + H]⁺ |
| INT 141 | 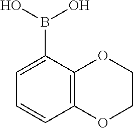 [SMILES: OB(O)c1cccc2c1OCCO2] | CAS: 499769-88-9 |
| INT 142 | 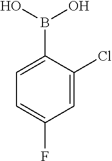 [SMILES: OB(O)c1ccc(F)cc1Cl] | CAS: 313545-72-1 |
| INT 143 | 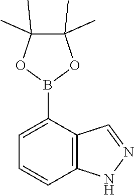 [SMILES: CC1(C)OB(c2cccc3[nH]ncc23)OC1(C)C] | ES+ (m/z) = 245.3 [M + H]⁺ Used as a 1:1 mixture with the de-brominated starting material. |
| INT 144 | 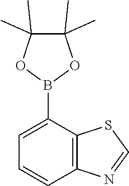 [SMILES: CC1(C)OB(c2cccc3ncsc23)OC1(C)C] | CAS: 1326714-48-0 |
| INT 145 | 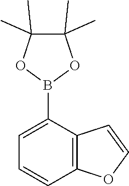 [SMILES: CC1(C)OB(c2cccc3occc23)OC1(C)C] | CAS: 915412-92-9 |
| INT 146 | 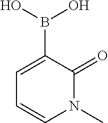 | CAS: 1454558-46-3 |
| INT 147 | 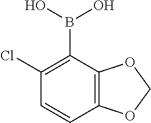 [SMILES: OB(O)c1c(Cl)ccc2c1OCO2] | CAS: 2087452-50-2 |
| INT 148 | 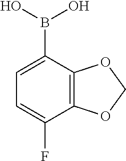 [SMILES: OB(O)c1ccc(F)c2c1OCO2] | CAS: 1256345-92-2 |
| INT 149 | 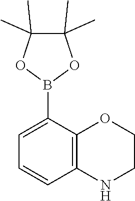 [SMILES: CC1(C)OB(c2cccc3c2OCCN3)OC1(C)C] | CAS: 2377610-89-2 |
| INT 150 | 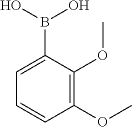 | CAS: 40972-86-9 |
| INT 151 | 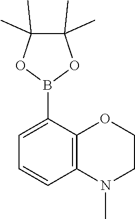 [SMILES: CN1CCOc2c(B3OC(C)(C)C(C)(C)O3)cccc21] | CAS: 1583286-76-3 |
| INT 152 | 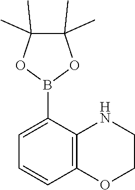 [SMILES: CC1(C)OB(c2cccc3c2NCCO3)OC1(C)C] | ES+ (m/z) = 262.3 [M + H]⁺ ¹H NMR (400 MHz, CDCl₃) δ 7.22 (dd, J = 1.5, 7.4 Hz, 1H), 6.84 (dd, J = 1.5, 7.8 Hz, 1H), 6.56 (t, J = 7.6 Hz, 1H), 4.23 (t, J = 4.4 Hz, 2H), 3.48 (t, J = 4.4 Hz, 2H), 1.34 (s, 12H). NH not seen. |
| INT 153 | 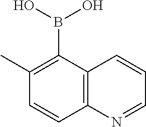 | CAS: 1287753-42-7 |
| INT 154 | 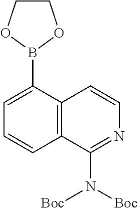 [SMILES: CC(C)(C)OC(=O)N(C(=O)OC(C)(C)C)c1nccc2c(B3OCCO3)cccc12] | ES+ (m/z) = 471.6 [M + H]⁺ ¹H NMR (400 MHz, CDCl₃) δ 8.56 (d, J = 6.0 Hz, 1H), 8.45 (d, J = 6.0 Hz, 1H), 8.28 (dd, J = 1.2, 6.8 Hz, 1H), 8.06 (d, J = 8.4 Hz, 1H), 7.60 (dd, J = 6.8, 8.4 Hz, 1H), 1.44 (s, 12H), 1.28 (s, 18H) |
| INT 155 | 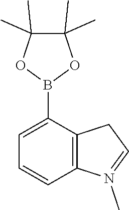 | CAS: 898289-06-0 |
| INT 156 | 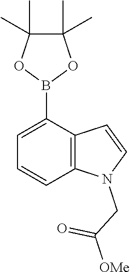 | ES⁺ (m/z) = 330.4 [M + H]⁺ ¹H NMR (400 MHz, CDCl₃) δ 7.64 (dd, J = 0.7, 7.0 Hz, IH), 7.36 (d, J = 8.1 Hz, 1H), 7.22 (dd, J = 7.0, 8.1 Hz, 1H), 7.14 (d, J = 3.2 Hz, 1H), 7.06 (dd, J-0.7, 3.2 Hz, 1H), 4.84 (s, 2H), 1.38 (s, 12H), 1.23 (t, J = 7.1 Hz, 3H) |
| INT 157 | 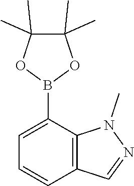 [SMILES: Cn1ncc2cccc(B3OC(C)(C)C(C)(C)O3)c21] | CAS: 1313738-64-5 |
| INT 158 | 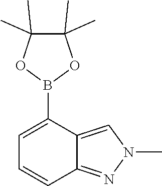 | CAS: 885698-95-3 |
| INT 159 | 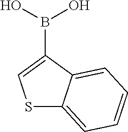 [SMILES: OB(O)c1csc2ccccc12] | CAS: 113893-08-6 |
| INT 160 | 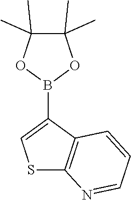 [SMILES: CC1(C)OB(c2csc3ncccc23)OC1(C)C] | ES+ (m/z) = 262.1 [M + H]⁺ ¹H NMR (400 MHz, CDCl₃) d 8.58 (dd, J = 1.7, 8.1 Hz, 1H), 8.55 (dd, J = 1.6, 4.7 Hz, 1H), 8.15 (s, 1H), 7.33 (dd, J = 4.6, 7.8 Hz, 1H), 1.38 (s, 12H). |
| INT 161 | 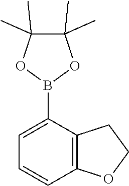 [SMILES: CC1(C)OB(c2cccc3c2CCO3)OC1(C)C] | CAS: 2246779-62-2 |
| INT 162 | 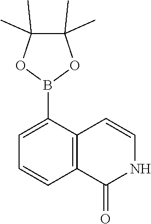 [SMILES: CC1(C)OB(c2cccc3c(=O)[nH]ccc23)OC1(C)C] | ES+ (m/z) = 272.3 [M + H]⁺ ¹H NMR (400 MHz, CDCl₃) d 10.67 (s, 1H), 8.55 (dd, J = 1.2, 8.0 Hz, 1H), 8.23 (dd, J = 1.4, 7.2 Hz, 1H), 7.55- 7.47(m, 2H), 7.15 (d, J = 7.2 Hz, 1H), 1.40 (s, 12H) |
| INT 163 | 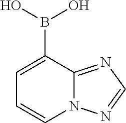 [SMILES: OB(O)c1cccn2ncnc12] | ES+ (m/z) = 164.1 [M + H]⁺ |
| INT 164 | 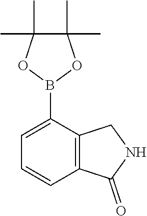 [SMILES: CC1(C)OB(c2cccc3c2CNC3=O)OC1(C)C] | ES+ (m/z) = 260.3 [M + H]⁺ Used crude as mixture with boronic acid |
| INT 165 | 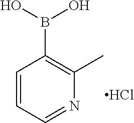 | CAS: 899436-71-6 |
| INT 166 | 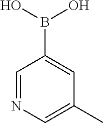 | CAS: 173999-18-3 |
| INT 167 | 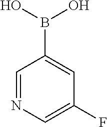 | CAS: 872041-86-6 |
| INT 168 | 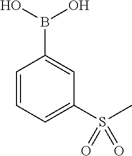 | CAS: 373384-18-0 |
| INT 169 | 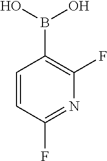 [SMILES: OB(O)c1ccc(F)nc1F] | CAS: 136466-94-9 |
| INT 170 | 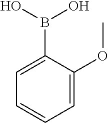 | CAS 5720-06-9 |
| INT 171 | 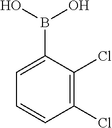 [SMILES: OB(O)c1cccc(Cl)c1Cl] | CAS 151169-74-3 |
| INT 172 | 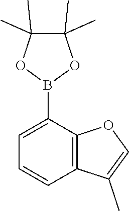 [SMILES: Cc1coc2c(B3OC(C)(C)C(C)(C)O3)cccc12] | ES+ (m/z) = no ion peak observed ¹H NMR (400 MHz, CDCl₃) d 7.76 − 7.73 (m, 1H), 7.64 − 7.61 (m, 1H), 7.51 − 7.50 (m, 1H), 7.28 − 7.24 (m, 1H), 2.23 (s, 3H), 1.41 (s, 12H). |
| INT 173 | 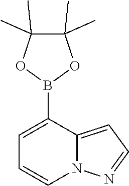 [SMILES: CC1(C)OB(c2cccn3nccc23)OC1(C)C] | CAS 2628351-17-5 |
| INT 174 | 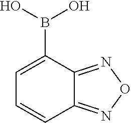 [SMILES: OB(O)c1cccc2nonc12] | CAS: 207279-32-1 |
| INT 175 | 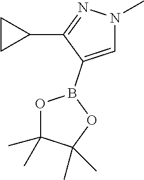 [SMILES: Cn1cc(B2OC(C)(C)C(C)(C)O2)c(C2CC2)n1] | CAS: 1257637-82-3 |
| INT 176 | 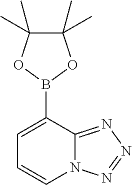 [SMILES: CC1(C)OB(c2cccn3nnnc23)OC1(C)C] | CAS: 1433983-02-8 |
| INT 177 | 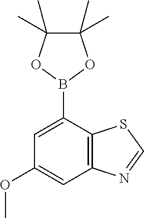 [SMILES: COc1cc(B2OC(C)(C)C(C)(C)O2)c2scnc2c1] | (ES, m/z): [M + H]⁺ 292.0, ¹H NMR (400 MHz, CDCl₃) δ 9.01 (s, 1H), 7.71 (d, J = 2.6 Hz, 1H), 7.55 (d, J = 2.6 Hz, 1H), 3.92 (s, 3H), 1.39 (s, 12H) |
| INT 178 | 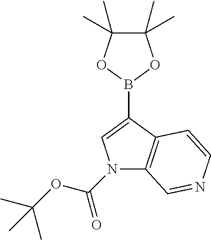 [SMILES: CC(C)(C)OC(=O)n1cc(B2OC(C)(C)C(C)(C)O2)c2ccncc21] | (ES, m/z): [M + H]⁺ 345.1 ¹H NMR (400 MHz, CDCl₃) d 9.40 (s, 1H), 8.43 (d, J = 5.4 Hz, 1H), 8.12 (s, 1H), 7.88 (d, J = 5.3 Hz, 1H), 1.69 (s, 9H), 1.38 (s, 12H) |
| INT 179 | 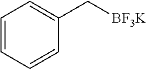 | CAS: 371766-08-4 |
| INT 180 | 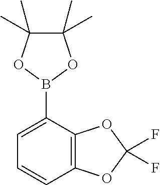 [SMILES: CC1(C)OB(c2cccc3c2OC(F)(F)O3)OC1(C)C] | CAS: 126120-87-4 |
| INT 181 | 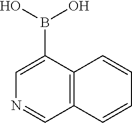 [SMILES: OB(O)c1cncc2ccccc12] | CAS: 192182-56-2 |
| INT 182 | 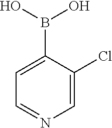 | CAS: 458532-98-4 |
| INT 211 | 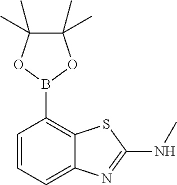 | (ES, m/z): [M + H]+ 291.1; ¹H NMR (400 MHz, DMSO) d 7.83 (dd, J = 4.6, 9.4 Hz, 1H), 7.50 (dd, J = 1.3, 7.7 Hz, 1H), 7.32 (dd, J = 1.3, 7.3 Hz, 1H), 7.23 (t, J = 7.6 Hz, 1H), 2.94 (d, J = 4.5 Hz, 3H), 1.32 (s, 12H). |
| INT 212 | 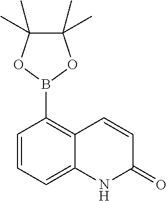 [SMILES: CC1(C)OB(c2cccc3[nH]c(=O)ccc23)OC1(C)C] | CAS: 1219130-53-6 |
| INT 213 | 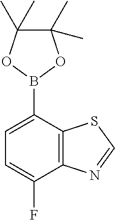 [SMILES: CC1(C)OB(c2ccc(F)c3ncsc23)OC1(C)C] | (ES, m/z): [M + H]+ 280.0 |
| INT 214 | 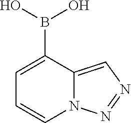 [SMILES: OB(O)c1cccn2nncc12] | (ES, m/z): [M + H]+ 163.9 |
| INT 215 | 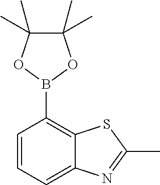 | (ES, m/z): [M + H]+ 276 |
| INT 216 | 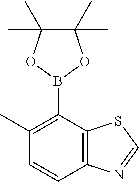 [SMILES: Cc1ccc2ncsc2c1B1OC(C)(C)C(C)(C)O1] | (ES, m/z): [M + H]+ 276.2 |

The pyrazole ester intermediates in Table 19 below were either synthesized or obtained from commercial sources.

TABLE 19

| Intermediate | | |
|---|---|---|
| No. | Structure | Analytical data |
| INT 183 | 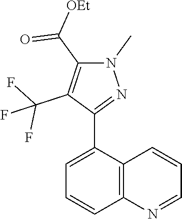 [SMILES: CCOC(=O)c1c(C(F)(F)F)c(-c2cccc3ncccc23)nn1C] | (ES, m/z): [M + H]⁺ 350.0 ¹H NMR (400 MHz, DMSO) 8.96 (1H, dd, J = 1.6, 4.1 Hz), 8.16 (1H, d, J = 8.4 Hz), 8.05 (1H, d, J = 8.2 Hz), 7.86 (1H, dd, J = 7.1, 8.5 Hz), 7.61 (1H, dd, J = 1.1, 7.2 Hz), 7.55 (1H, dd, J = 4.1, 8.5 Hz), 4.44 (2H, q, J = 7.1 Hz), 4.19 (3H, s), 1.35 (3H, t, J = 7.1 Hz) |
| INT 184 | 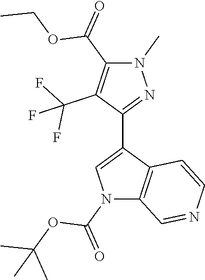 [SMILES: CCOC(=O)c1c(C(F)(F)F)c(-c2cn(C(=O)OC(C)(C)C)c3cnccc23)nn1C] | (ES, m/z): [M + H]⁺ 439.2 ¹H NMR (400 MHz, CDCl₃) d 9.45 (s, 1H), 8.47 (d, J = 5.4 Hz, 1H), 7.98 (s, 1H), 7.78 (d, J = 5.4 Hz, 1H), 4.47 (q, J = 7.2 Hz, 2H), 4.20 (s, 3H), 1.72 (s, 9H), 1.44 (t, J = 7.2 Hz, 3H) |
| INT 185 | 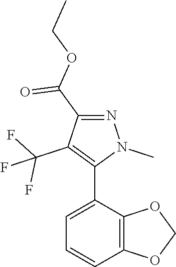 [SMILES: CCOC(=O)c1nn(C)c(-c2cccc3c2OCO3)c1C(F)(F)F] | (ES, m/z): [M + H]^{\|} 343.0, ¹H NMR (400 MHz, DMSO) δ 7.14 (dd, J = 1.2, 7.8 Hz, 1H), 7.02 ( t, J = 7.9 Hz, 1H), 6.94 (dd, J = 1.2, 7.7 Hz, 1H), 6.12 (s, 2H), 4.35 (q, J = 7.1 Hz, 2H), 3.78 (s, 3H), 1.32 (t, J = 7.1 Hz, 3H) |
| INT 186 | 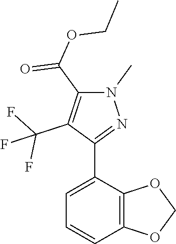 [SMILES: CCOC(=O)c1c(C(F)(F)F)c(-c2cccc3c2OCO3)nn1C] | (ES, m/z): [M + H]⁺ 343.3, ¹H NMR (400 MHz, DMSO) δ 7.04 (dd, J = 1.4, 7.6 Hz, 1H), 6.93 (t, J = 7.8 Hz, 1H), 6.88 (dd, J = 1.4, 7.9 H7, 1H), 6.04 (s, 2H), 4.41 (q, J = 7.1 Hz, 2H), 4.14 (s, 3H), 1.34 (t, J = 7.1 Hz, 3H) |
| INT 187 | 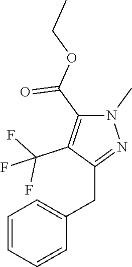 [SMILES: CCOC(=O)c1c(C(F)(F)F)c(Cc2ccccc2)nn1C] | (ES, m/z): [M + H]⁺ 313.0 |
| INT 188 | 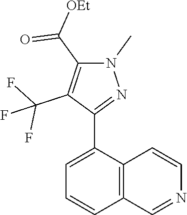 [SMILES: CCOC(=O)c1c(C(F)(F)F)c(-c2cccc3cnccc23)nn1C] | (ES, m/z): [M + H]⁺ 350.0 |
| INT 189 | 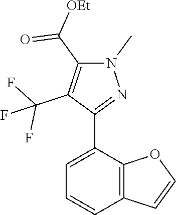 [SMILES: CCOC(=O)c1c(C(F)(F)F)c(-c2cccc3ccoc23)nn1C] | (ES, m/z): [M + H]⁺ 339.3, ¹H NMR (400 MHz, CDCl₃) δ 7.67 (dd, J = 1.1, 7.6 Hz, 1H), 7.64 (d, J = 2.2 Hz, 1H), 7.37 (d, J = 6.7 Hz, 1H), 7.30 (t, J = 7.6 Hz, 1H), 6.82 (d, J = 2.2 Hz, 1H), 4.46 (q, J = 7.1 Hz, 2H), 4.24 (s, 3H), 1.43 (t, J = 7.1 Hz, 3H) |
| INT 190 | 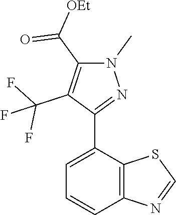 [SMILES: CCOC(=O)c1c(C(F)(F)F)c(-c2cccc3ncsc23)nn1C] | (ES, m/z): [M + H]⁺, 356.3, ¹H NMR (400 MHz, CDCl₃) δ 9.04 (s, 1H), 8.19 (dd, J = 1.5, 7.7 Hz, 1H), 7.60 (t, J = 7.6 Hz, 1H), 7.56 (br d, J = 7.2 Hz, 1H), 4.48 (q, J = 7.1 Hz, 2H), 4.22 (s, 3H), 1.44 (t, J = 7.1 Hz, 3H) |
| INT 191 | 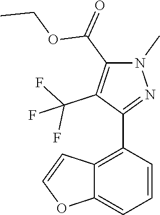 [SMILES: CCOC(=O)c1c(C(F)(F)F)c(-c2cccc3occc23)nn1C] | (ES, m/z): [M + H]⁺ 339.0, ¹H NMR (400 MHz, CDCl₃) d 7.66 (d, J = 2.2 Hz, 1H), 7.58-7.54 (m, 1H), 7.38-7.30 (m, 2H), 6.82 (d, J = 1.4 Hz, 1H), 4.47 (q, J = 7.2 Hz, 2H), 4.21 (s, 3H), 1.44 (t, J = 7.2 Hz, 3H) |
| INT 192 | 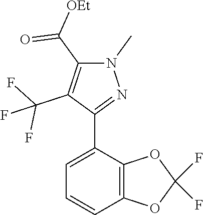 [SMILES: CCOC(=O)c1c(C(F)(F)F)c(-c2cccc3c2OC(F)(F)O3)nn1C] | (ES, m/z): [M + H]⁺ 378.9, 378.9, ¹H NMR (400 MHz, CDCl₃) d 7.18-7.12 (m, 3H), 4.48-4.42 (m, 2H), 4.22 (s, 3H), 1.42 (t, J = 7.2 Hz, 3H) |
| INT 193 | 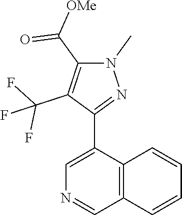 [SMILES: COC(=O)c1c(C(F)(F)F)c(-c2cncc3ccccc23)nn1C] | (ES, m/z): [M + H]⁺ 336.3 |
| INT 194 | 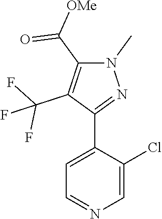 [SMILES: COC(=O)c1c(C(F)(F)F)c(-c2ccncc2Cl)nn1C] | (ES, m/z): [M + H]⁺ 320.2 ¹H NMR (400 MHz, CDCl₃) d 8.71 (s, 1H), 8.57 (d, J = 3.8 Hz, 1H), 7.30 (d, J = 4.1 Hz, 1H), 4.26 (s, 3H), 4.01 (s, 3H) |
| INT 195 | 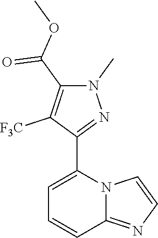 [SMILES: COC(=O)c1c(C(F)(F)F)c(-c2cccc3nccn23)nn1C] | ES+ (m/z) [M + H]⁺ 325.0 ¹H NMR (400 MHz, DMSO) 7.73 (d, J = 9.0 Hz, 1H), 7.65 (d, J = 10.6 Hz, 2H), 7.38-7.34 (m, 1H), 7.01 (d, J = 6.5 Hz, 1H), 4.21 (s, 3H), 3.98 (s, 3H) |
| INT 217 | 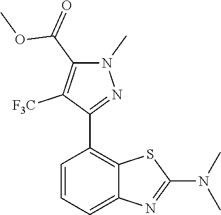 | (ES, m/z): [M + H]+ 385.1; ¹H NMR (400 MHz, CDCl₃) d 7.61-7.58 (m, 1H), 7.34 (dd, J = 7.8, 7.8 Hz, 1H), 7.15 (d, J = 7.3 Hz, 1H), 4.20 (s, 3H), 4.01 (s, 3H), 3.21 (s, 6H). |
| INT 218 | 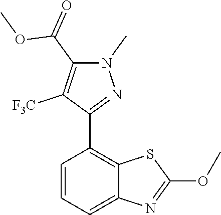 | (ES, m/z): [M + H]+ 370.2, H NMR (400 MHz, CDCl₃) 7.73 (1H, dd, J = 1.1, 8.0 Hz), 7.43 (1H, dd, J = 7.8, 7.8 Hz), 7.36 (1H, td, J = 1.1, 7.7 Hz), 4.20 (3H, s), 4.19 (3H, s), 4.01 (3H, s). |
| INT 219 | 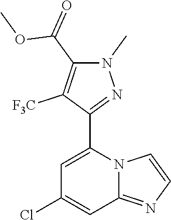 [SMILES: COC(=O)c1c(C(F)(F)F)c(-c2cc(Cl)cc3nccn23)nn1C] | (ES, m/z): [M + H]+ 359.0, ¹H NMR (400 MHz, CDCl₃) d 7.71 (d, J = 1.8 Hz, 1H), 7.63 (s, 1H), 7.44 (s, 1H), 6.88 (d, J = 1.9 Hz, 1H), 4.28 (s, 3H), 4.04 (s, 3H). |
| INT 220 | 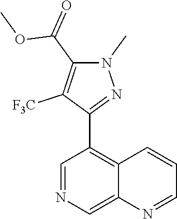 [SMILES: COC(=O)c1c(C(F)(F)F)c(-c2cncc3ncccc23)nn1C] | (ES, m/z): [M + H]+ 337.0, ¹H NMR (400 MHz, CDCl₃) d 9.60 (s, 1H), 9.07 (dd, J = 1.6, 4.2 Hz, 1H), 8.64 (s, 1H), 8.11 (d, J = 8.6 Hz, 1H), 7.62 (dd, J = 4.3, 8.6 Hz, 1H), 4.28 (s, 3H), 4.04 (s, 3H). |
| INT 221 | 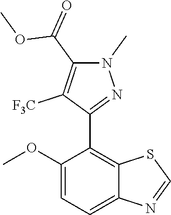 [SMILES: COC(=O)c1c(C(F)(F)F)c(-c2c(OC)ccc3ncsc23)nn1C] | (ES, m/z): [M + H]+ 372.0, ¹H NMR (400 MHz, CDCl₃) d 8.83 (s, 1H), 8.12 (d, J = 9.0 Hz, 1H), 7.23-7.20 (m, 1H), 4.24 (s, 3H), 4.01 (s, 3H), 3.89 (s, 3H). |
| INT 222 | 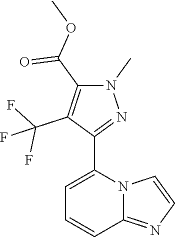 [SMILES: COC(=O)c1c(C(F)(F)F)c(-c2cccc3nccn23)nn1C] | (ES, m/z): [M + H]+ 325.0, ¹H NMR (400 MHz, DMSO) 7.73 (d, J = 9.0 Hz, 1H), 7.65 (d, J = 10.6 Hz, 2H), 7.38-7.34 (m, 1H), 7.01 (d, J = 6.5 Hz, 1H), 4.21 (s, 3H), 3.98 (s, 3H); |
| INT 223 | 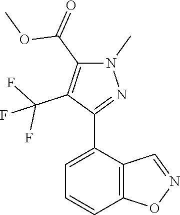 [SMILES: COC(=O)c1c(C(F)(F)F)c(-c2cccc3oncc23)nn1C] | (ES, m/z): [M + H]+ 325.0 |
| INT 224 | 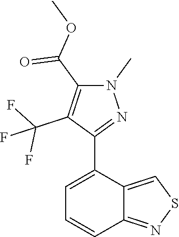 [SMILES: COC(=O)c1c(C(F)(F)F)c(-c2cccc3nscc23)nn1C] | (ES, m/z): [M + H]+ 341.9 ¹H NMR (400 MHz, CDCl3) δ 9.26 (d, J = 1.0 Hz, 1H), 7.92 (td, J = 0.9, 9.0 Hz, 1H), 7.51 (dd, J = 6.7, 9.0 Hz, 1H), 7.33 (d, J = 6.8 Hz, 1H), 4.24 (s, 3H), 4.03 (s, 3H). |
| INT 225 | 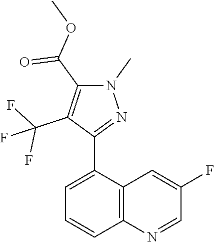 [SMILES: COC(=O)c1c(C(F)(F)F)c(-c2cccc3ncc(F)cc23)nn1C] | (ES, m/z): [M + H]+ 354.0, ¹H NMR (400 MHz, DMSO) 9.02 (d, J = 3.0 Hz, 1H), 8.22- 8.19 (m, 1H), 7.89 (dd, J = 2.7, 10.1 Hz, 1H), 7.85 (dd, J = 7.2, 8.4 Hz, 1H), 7.67 (d, J = 7.1 Hz, 1H), 4.18 (s, 3H), 3.98 (s, 3H); |
| INT 226 | 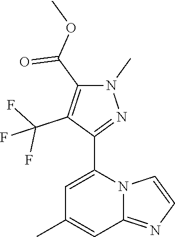 [SMILES: COC(=O)c1c(C(F)(F)F)c(-c2cc(C)cc3nccn23)nn1C] | (ES, m/z): [M + H]+ 339.0 |
| INT 227 | 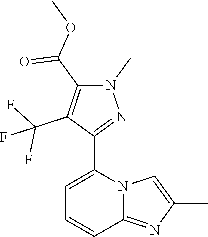 [SMILES: COC(=O)c1c(C(F)(F)F)c(-c2cccc3nc(C)cn23)nn1C] | (ES, m/z): [M + H]+ 339.0, ¹H NMR (400 MHz, CDCl3) d 7.60 (d, J = 8.9 Hz, 1H), 7.20 (dd, J = 7.0, 9.0 Hz, 1H), 6.81 (d, J = 6.9 Hz, 2H), 4.26 (s, 3H), 4.03 (s, 3H), 2.44 (s, 3H). |
| INT 228 | 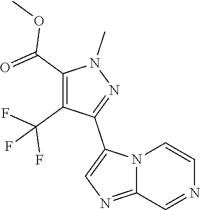 [SMILES: COC(=O)c1c(C(F)(F)F)c(-c2cnc3cnccn23)nn1C] | (ES, m/z): [M + H]+ 326.0, ¹H NMR (400 MHz, CDCl3) d 9.19 (d, J = 1.5 Hz, 1H), 8.72 (dd, J = 1.4, 4.7 Hz, 1H), 8.08 (d, J = 1.1 Hz, 1H), 8.00-7.98 (m, 1H), 4.23 (s, 3H), 4.04 (s, 3H). |
| INT 229 | 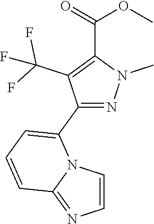 [SMILES: COC(=O)c1c(C(F)(F)F)c(-c2cccc3nccn23)nn1C] | (ES, m/z): [M + H]+ 325.0, ¹H NMR (400 MHz, CDCl3) d 8.08 (s, 1H), 7.55-7.52 (m, 2H), 6.79 (dd, J = 6.5, 9.2 Hz, 1H), 6.66-6.63 (m, 1H), 4.26 (s, 3H), 4.03 (s, 3H). |
| INT 230 | 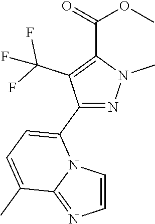 | (ES, m/z): [M + H]+ 339.0, ¹H NMR (400 MHz, CDCl3) d 7.63 (d, J = 1.1 Hz, 1H), 7.51 (s, 1H), 7.07-6.95 (m, 1H), 6.80 (d, J = 7.0 Hz, 1H), 4.26-4.26 (m, 3H), 4.03 (s, 3H), 2.67 (s, 3H). |
| INT 231 | 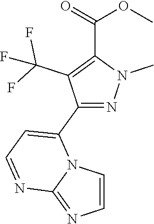 | (ES, m/z): [M + H]+ 339.0, ¹H NMR (400 MHz, CDCl3) d 8.65-8.63 (m, 1H), 7.87 (s, 1H), 7.64-7.62 (m, 1H), 6.96 (d, J = 4.3 Hz, 1H), 4.29 (s, 3H), 4.05 (s, 3H). |
| INT 232 | 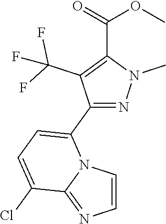 [SMILES: COC(=O)c1c(C(F)(F)F)c(-c2ccc(Cl)c3nccn23)nn1C] | (ES, m/z): [M + H]+ 359.0, ¹H NMR (400 MHz, CDCl3) d 7.70 (s, 1H), 7.58 (s, 1H), 7.35 (d, J = 7.5 Hz, 1H), 6.83 (d, J = 7.5 Hz, 1H), 4.27 (s, 3H), 4.04 (s, 3H). |
| INT 233 | 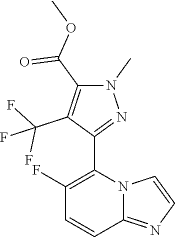 [SMILES: COC(=O)c1c(C(F)(F)F)c(-c2c(F)ccc3nccn23)nn1C] | (ES, m/z): [M + H]+ 343.1, ¹H NMR (400 MHz, CDCl3) d 7.73-7.67 (m, 2H), 7.45 (s, 1H), 7.22-7.16 (m, 1H), 4.31 (s, 3H), 4.04 (s, 3H). |
| INT 234 | 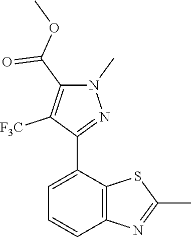 [SMILES: COC(=O)c1c(C(F)(F)F)c(-c2cccc3nc(C)sc23)nn1C] | (ES, m/z): [M + H]+ 356.0; ¹H NMR (400 MHz, DMSO-d6) δ 7.99 (dd, J = 1.7, 8.0 Hz, 1H), 7.51 (t, J = 7.8 Hz, 1H), 7.47-7.43 (m, 1H), 4.21 (s, 3H), 4.02 (s, 3H), 2.83 (s, 3H). |
| INT 235 | 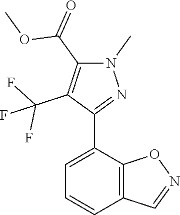 [SMILES: COC(=O)c1c(C(F)(F)F)c(-c2cccc3cnoc23)nn1C] | (ES, m/z): [M + H]+ 326.0 ¹H NMR (400 MHz, CDCl3) δ 10.02 (s, 1H), 7.74-7.69 (m, 1H), 7.58 (dd, J = 1.6, 7.8 Hz, 1H), 7.03 (t, J = 7.8 Hz, 1H), 4.15 (s, 3H), 4.03 (s, 3H). |

The tricyclic intermediates in Table 20 below were either synthesized following the procedures in Example 38.

TABLE 20

| Intermediate | | |
|---|---|---|
| No. | Structure | Analytical data |
| INT 196 | 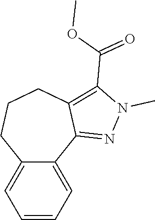 [SMILES: COC(=O)c1c2c(nn1C)-c1ccccc1CCC2] | ¹H NMR (400 MHz, CDCl₃) δ 7.97 (dd, J = 1.4, 7.5 Hz, 1H), 7.27 (dt, J = 1.7, 7.4 Hz, 1H), 7.22 (dt, J = 1.6, 7.3 Hz, 1H), 7.17 (dd, J = 1.4, 7.4 Hz, 1H), 4.21 (s, 3H), 3.93 (s, 3H), 2.99 (t, J = 6.9 Hz, 2H), 2.80-2.76 (m, 2H), 2.12-2.05 (m, 2H) |
| INT 197 | 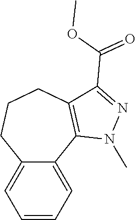 [SMILES: COC(=O)c1nn(C)c2c1CCCc1ccccc1-2] | ¹H NMR (400 MHz, CDCl₃) δ 7.35-7.33 (m, 4H), 4.04 (s, 3H), 3.95 (s, 3H), 2.81 (t, J = 7.4 Hz, 2H), 2.55 (t, J = 6.8 Hz, 2H), 2.19 (tt, J = 7.0, 7.1 Hz, 2H) |
| INT 198 | 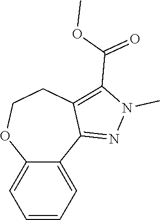 [SMILES: COC(=O)c1c2c(nn1C)-c1ccccc1OCC2] | ES+ (m/z) [M + H]⁺ 259.0, ¹H NMR (400 MHz, CDCl₃) δ 8.27 (dd, J = 1.8, 7.9 Hz, 1H), 7.20 (dt, J = 1.5, 7.6 Hz, 1H), 7.09 (dt, J = 1.3, 7.6 Hz, 1H), 7.02 (dd, J = 1.2, 8.0 Hz, 1H), 4.31 (t, J = 5.3 Hz, 2H), 4.22 (s, 3H), 3.93 (s, 3H), 3.29 (t, J = 5.3 Hz, 2H) |

### Example 54—Synthesis of 6-(2H-1,2,3-triazol-2-yl)-5-(trifluoromethyl)369yridine-3-amine—INT 200

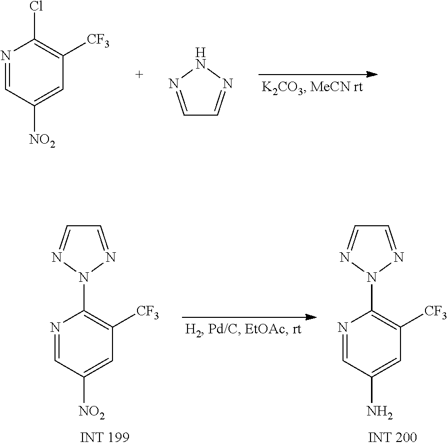 [SMILES: O=[N+]([O-])c1cnc(Cl)c(C(F)(F)F)c1 ; c1cn[nH]n1 ; O=[N+]([O-])c1cnc(-n2nccn2)c(C(F)(F)F)c1 ; Nc1cnc(-n2nccn2)c(C(F)(F)F)c1]

Step 1: Synthesis of 5-nitro-2-(2H-1,2,3-triazol-2-yl)-3-(trifluoromethyl)pyridine (INT 199): K₂CO₃ (7.6 g, 55.2 mmol, 2.50 eq) was added to a stirred mixture of 2-chloro-5-nitro-3-(trifluoromethyl)pyridine (3.1 mL, 22.1 mmol, 1.00 eq) and 2H-triazole (2.6 mL, 44.1 mmol, 2.00 eq) in MeCN (100 mL) and the reaction was stirred at rt for 48 hours. The crude mixture was partitioned between water and EtOAc. The organic layer was dried (MgSO₄) and concentrated under reduced pressure. The crude residue was purified by reverse phase column chromatography to give the title compound as an off-white solid (2.6 g, 45%). ¹H NMR (400 MHz, CDCl₃) δ 9.59 (d, J=2.4 Hz, 1H), 9.06 (d, J=2.4 Hz, 1H), 8.06 (s, 2H).

Step 2: Synthesis of 6-(2H-1,2,3-triazol-2-yl)-5-(trifluoromethyl)pyridin-3-amine (INT 200): Palladium on carbon (10% w/w wet support) (1.2 g, 0.579 mmol, 0.1000 eq) was added to a degassed solution of 5-nitro-2-(triazol-2-yl)-3-(trifluoromethyl)pyridine (1.5 g, 5.79 mmol, 1.00 eq) in EtOAc (60 mL). The resulting mixture was stirred under one atmosphere of H₂ for 16 hours. The reaction mixture was filtered through Celite®, which was washed with EtOAc. The filtrate was concentrated under reduced pressure to give the title compound as a dark green solid (2.0 g, 87%). ¹H NMR (400 MHz, CDCl₃) δ 8.13 (d, J=2.5 Hz, 1H), 7.88 (s, 2H), 7.37 (d, J=2.6 Hz, 1H), 4.29 (s, 2H).

### Example 55—Synthesis of 5-(difluoromethyl)-6-(2H-1,2,3-triazol-2-yl) 370 yridine-3-amine—INT 204

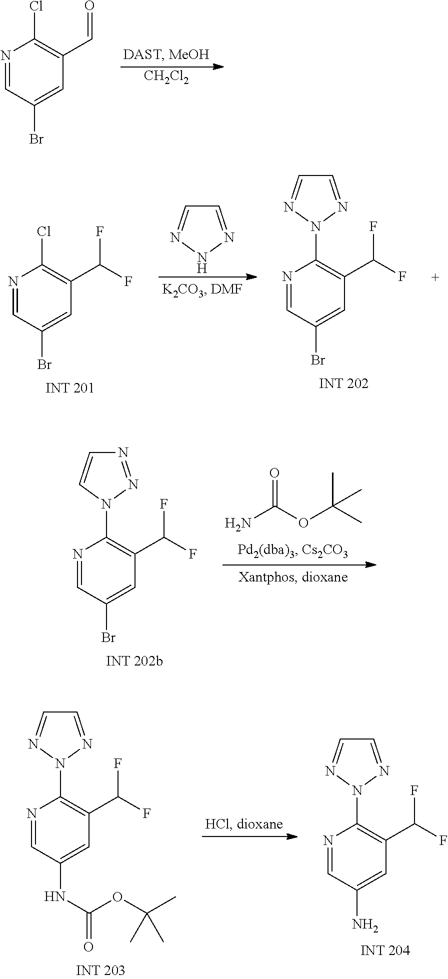

Step 1: Synthesis of 5-bromo-2-chloro-3-(difluoromethyl)pyridine (INT 201): (Diethylamino) sulfur trifluoride (6.6 mL, 49.9 mmol, 2.20 eq) was added dropwise to a solution of 5-bromo-2-chloronicotinaldehyde (5.0 g, 22.7 mmol, 1.00 eq) in DCM (150 mL) at 0° C. followed by the addition of MeOH (0.0092 mL, 0.227 mmol, 0.0100 eq). The reaction was stirred at 0° C. for 90 minutes. The reaction mixture was quenched with saturated aqueous NaHCO₃ and stirred at 0° C. until effervescence stopped. The aqueous layer was washed with DCM and the combined organic phases were dried over a phase separator cartridge, concentrated under reduced pressure and purified by silica gel chromatography to give the title compound as a yellow oil (4.9 g, 90%). ¹H NMR (400 MHz, CDCl₃) δ 8.59-8.57 (m, 1H), 8.12 (d, J=2.4 Hz, 1H), 6.88 (t, J=54.1 Hz, 1H).

Step 2: Synthesis of 5-bromo-3-(difluoromethyl)-2-(2H-1,2,3-triazol-2-yl)pyridine (INT 202): K₂CO₃ (7.0 g, 51.1 mmol, 2.50 eq) was added to a solution of 5-bromo-2-chloro-3-(difluoromethyl)pyridine (4.9 g, 20.4 mmol, 1.00 eq) and 1H-1,2,3-triazole (1.4 mL, 24.5 mmol, 1.20 eq) in DMF (100 mL) and the reaction was stirred at 90° C. for 16 hours, cooled to rt and diluted with water, brine and EtOAc. The organic layer was separated and washed with 4% aqueous LiCl solution, brine, dried (Na₂SO₄) and concentrated under reduced pressure. The crude residue was purified by silica gel chromatography eluting with 0-100% EtOAc in cyclohexane to give the title compound and 5-bromo-3-(difluoromethyl)-2-(1H-1,2,3-triazol-1-yl)pyridine as a 1.3:1 mixture (4.2 g), which was used in the next step without further purification. ¹H NMR (400 MHz, CDCl₃) δ 8.77 (d, J=2.1 Hz, 1H), 8.38 (d, J=2.3 Hz, 1H), 7.97 (s, 2H), 7.57 (t, J=54.6 Hz, 1H).

Step 3: Synthesis of tert-butyl (5-(difluoromethyl)-6-(2H-1,2,3-triazol-2-yl) pyridin-3-yl) carbamate (INT 203): Pd₂(dba); (279 mg, 0.304 mmol, 0.0200 eq) was added to a stirred degassed solution of a 1.3:1 mixture of 5-bromo-3-(difluoromethyl)-2-(2H-1,2,3-triazol-2-yl)pyridine and 5-bromo-3-(difluoromethyl)-2-(1H-1,2,3-triazol-1-yl)pyridine (4.2 g, 15.2 mmol, 1.00 eq), Xantphos (352 mg, 0.609 mmol, 0.0400 eq), Cs₂CO₃ (14.8 g. 45.6 mmol, 3.00 eq) and tert-butyl carbamate (2.0 g, 16.7 mmol, 1.10 eq) in 1,4-dioxane (100 mL). The reaction was stirred at 105° C. for 16 hours, then it was cooled to rt, diluted with EtOAc and filtered through Celite®, which was washed with EtOAc. The solvent was removed under reduced pressure and the crude residue was purified by silica gel chromatography to give the title compound as a yellow gum (427 mg, 9%). ¹H NMR (400 MHz, CDCl₃) δ 8.59-8.57 (m, 1H), 8.52 (s, 1H), 7.92 (s, 2H), 7.46 (t, J=55.0 Hz, 1H), 6.84 (s, 1H), 1.55 (s, 9H).

Step 4: Synthesis of 5-(difluoromethyl)-6-(2H-1,2,3-triazol-2-yl) pyridin-3-amine (INT 204): 4 M HCl in 1,4-dioxane (3.4 mL, 13.7 mmol, 10.0 eq) was added to a solution of tert-butyl (5-(difluoromethyl)-6-(2H-1,2,3-triazol-2-yl) pyridin-3-yl) carbamate (427 mg, 1.37 mmol, 1.00 eq) in DCM (10 mL) and the reaction was stirred at rt for 90 minutes. The solvent was removed under reduced pressure and the residue was partitioned between DCM and a mixture of water and saturated aqueous NaHCO₃. The aqueous layer was separated and washed with DCM and the combined organic layers were dried over a phase separator cartridge and concentrated under reduced pressure. The crude residue was purified by column chromatography to give the title compound as a dark brown solid (246 mg, 85%). ¹H NMR (400 MHz, CDCl₃) δ 8.12 (d, J=2.8 Hz, 1H), 7.88 (s, 2H), 7.45 (d, J=2.7 Hz, 1H), 7.30 (t, J=54.9 Hz, 1H), 4.09 (s, 2H).

### Example 56—Synthesis of 5-amino-2-methoxynicotinonitrile—INT 206

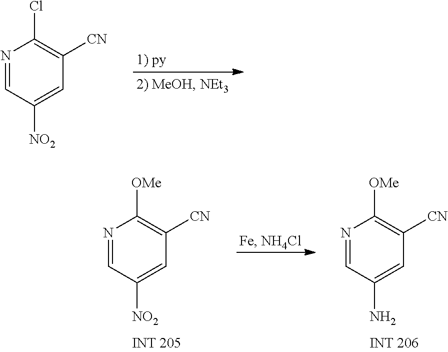

Step 1: Synthesis of 2-methoxy-5-nitronicotinonitrile (INT 205): Pyridine (1.3 mL, 16.3 mmol, 3.00 eq) was added to a solution of 2-chloro-5-nitronicotinonitrile (1,000 mg, 5.45 mmol, 1.00 eq) and in MeCN (30 mL), the reaction was stirred at rt for 2.5 hours. The solids were filtered, washed with cold MeCN and re-dissolved in MeOH (30 mL). Et₃N (0.76 mL, 5.45 mmol, 1.00 eq) was added and the reaction was stirred at rt for 1 hour. Upon addition of water, a precipitate formed, which was filtered and washed with water to give the title compound as a purple solid (756 mg, 4.22 mmol, 77%). ¹H NMR (400 MHz, CDCl₃) δ 9.23 (d, J=2.8 Hz, 1H), 8.69 (d, J=2.6 Hz, 1H), 4.21 (s, 3H).

Step 2: Synthesis of 5-amino-2-methoxynicotinonitrile (INT 206): Iron powder (1.09 g, 19.5 mmol, 5.00 eq) was added to a mixture of 2-methoxy-5-nitronicotinonitrile (700 mg, 3.91 mmol, 1.00 eq) and NHCl (1.05 g, 19.5 mmol, 5.00 eq) in MeOH (1.5 mL), THF (3 mL), and water (0.75 mL), the reaction mixture stirred at 60° C. for 3 hours. The reaction was cooled to rt, diluted with EtOAc and filtered. The filtrate was concentrated under reduced pressure. The crude material was redissolved in EtOAc and washed with saturated aqueous NaHCO₃. The organics were dried (Na₂SO₄), filtered, and concentrated under reduced pressure. The product was purified by column chromatography eluting with 0 to 100% EtOAc in cyclohexane to give the title compound as an off-white solid (3.74 mmol, 96%). ¹H NMR (400 MHz, CDCl₃) δ 7.84 (d, J=3.0 Hz, 1H), 7.24 (d, J=3.0 Hz, 1H), 3.97 (s, 3H), 3.55-3.53 (m, 2H).

### Example 57—Synthesis of 5-methyl-6-(2H-1,2,3-triazol-2-yl) pyridin-3-amine and 5-methyl-6-(1H-1,2,3-triazol-1-yl) pyridin-3-amine—INT 209

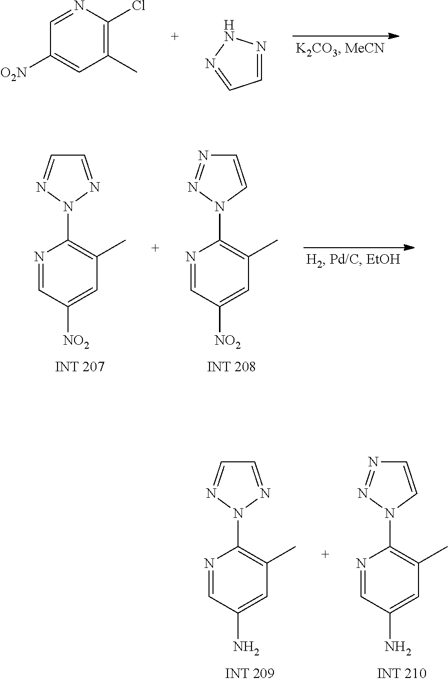

Step 1: Synthesis of 3-methyl-5-nitro-2-(2H-1,2,3-triazol-2-yl)pyridine and 3-methyl-5-nitro-2-(1H-1,2,3-triazol-1-yl)pyridine (INT 207): K₂CO₃ (10.0 g, 72.4 mmol, 2.66 eq) was added to a mixture of 2-Chloro-3-methyl-5-nitropyridine (4.70 g, 27.2 mmol, 1.00 eq) and 2H-triazole (3.3 mL, 57.0 mmol, 2.09 eq) in MeCN (70 mL), the reaction was stirred at 40° C. for 2 hours. The reaction was filtered, and the solvent removed under reduced pressure. The crude residue was partitioned between EtOAc (50 mL) and water (20 mL) and stirred at 40° C. until dissolution had occurred. The aqueous layer was separated and extracted with EtOAc, the combined organic phases were dried (MgSO₄) and concentrated under reduced pressure. The crude residue was purified by column chromatography eluting with 5-100% EtOAc in cyclohexane give a 1.3:1 mixture of 3-methyl-5-nitro-2-(2H-1,2,3-triazol-2-yl)pyridine INT 207 and 3-methyl-5-nitro-2-(1H-1,2,3-triazol-1-yl)pyridine INT 208 as an off-white solid (3.85 g, 18.8 mmol, 69%).

Step 2: Synthesis of 5-methyl-6-(2H-1,2,3-triazol-2-yl)373 yridine-3-amine and 5-methyl-6-(1H-1,2,3-triazol-1-yl)373 yridine-3-amine (INT 209): Palladium on carbon (10% w/w wet support, 380 mg, 0.18 mmol, 0.03 eq) was added to a degassed solution of a 1.3:1 mixture of 3-methyl-5-nitro-2-(2H-1,2,3-triazol-2-yl)pyridine and 3-methyl-5-nitro-2-(1H-1,2,3-triazol-1-yl)pyridine (1.24 g, 6 mmol, 1.00 eq) in EtOH (40 mL). The resulting mixture was stirred under one atmosphere of H₂ for 16 hours. The reaction mixture was filtered through Celite®, which was washed with EtOH. The filtrate was concentrated under reduced pressure and the crude residue was purified by reverse phase chromatography to give the title compound as an off-white solid (371 mg, 2.1 mmol, 35%). ¹H NMR (400 MH, CDCl₃) δ 8.08 (d, J=1.0 Hz, 1H), 7.82 (d, J=2.8 Hz, 1H), 7.81 (d, J=1.0 Hz, 1H), 7.01 (d, J=2.6 Hz, 1H), 3.90 (s, 2H), 2.38 (s, 3H). By-product 5-Methyl-6-(2H-1,2,3-triazol-2-yl)373yridine-3-amine—INT 210 was also isolated by reverse phase chromatography as a straw coloured oil (505 mg, 2.8 mmol, 47%). ¹H NMR (400 MHz, CDCl₃) δ 7.88 (d, J=2.8 Hz, 1H), 7.83 (s, 2H), 6.97 (d, J=2.6 Hz, 1H), 3.90 (s, 2H), 2.27 (s, 3H).

Compounds in Table 21 were prepared using a procedure analogous to that described in Example 34.

TABLE 21

| Compound | | | |
|---|---|---|---|
| No. | Structure | Starting Material | Analytical Data |
| I-231 | 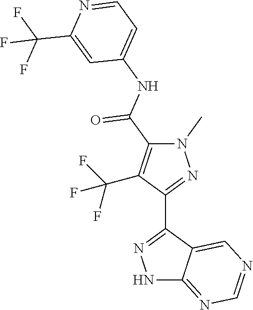 [SMILES: Cn1nc(-c2n[nH]c3ncncc23)c(C(F)(F)F)c1C(=O)Nc1ccnc(C(F)(F)F)c1] | 3-bromo-1H- pyrazolo[3,4- d]pyrimidine | (ES, m/z): [M + H]+ 457.2, ¹H NMR (400 MHz, DMSO) 14.49 (s, 1H), 11.99 (s, 1H), 9.62 (s, 1H), 9.10 (s, 1H), 8.76 (d, J = 5.6 Hz, 1H), 8.19 (d, J = 1.8 Hz, 1H), 7.89 (dd, J = 1.6, 5.4 Hz, 1H), 4.09- 4.08 (m, 3H). |
| I-243 | 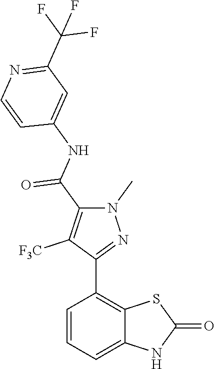 [SMILES: Cn1nc(-c2cccc3[nH]c(=O)sc23)c(C(F)(F)F)c1C(=O)Nc1ccnc(C(F)(F)F)c1] | 7- bromobenzo[d]thiazol- 2(3H)-one | (ES⁺, m/z): [M + H]⁺ 488. ¹H NMR (400 MHz, DMSO) 12.01 (s, 2H), 8.76 (d, J = 5.5 Hz, 1H), 8.18 (d, J = 1.6 Hz, 1H), 7.88 (dd, J = 1.6, 5.6 Hz, 1H), 7.45 (dd, J = 7.9, 7.9 Hz, 1H), 7.31 (d, J = 7.8 Hz, 1H), 7.23 (dd, J = 0.9, 7.9 Hz, 1H), 4.03 (s, 3H). |

### Example 58—N-(3-Chloro-4-(3-hydroxyazetidine-1-carbonyl)phenyl)-3-(imidazo[1,2-a]pyridin-5-yl)-1-methyl-4-(trifluoromethyl)-1H-pyrazole-5-carboxamide (I-241)

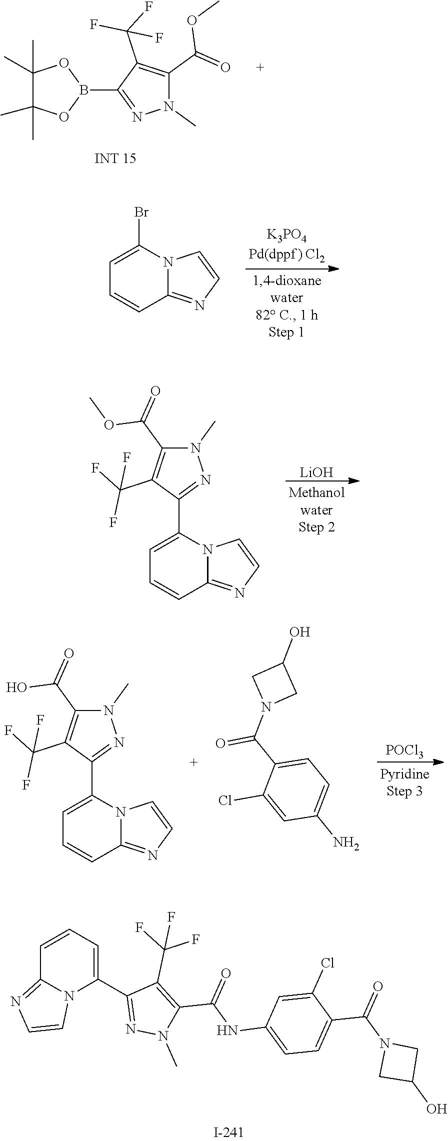

N-(3-Chloro-4-(3-hydroxyazetidine-1-carbonyl)phenyl)-3-(imidazo[1,2-a]pyridin-5-yl)-1-methyl-4-(trifluoromethyl)-1H-pyrazole-5-carboxamide (I-241) was prepared using procedures outlined in Examples 8 and 12 starting from 5-bromoimidazo[1,2-a]pyridine, methyl 1-methyl-3-(4,4,5,5-tetramethyl-1,3,2-dioxaborolan-2-yl)-4-(trifluoromethyl)-1H-pyrazole-5-carboxylate (INT 15) and (4-amino-2-chlorophenyl)-(3-hydroxyazetidin-1-yl) methanone to give N-(3-Chloro-4-(3-hydroxyazetidine-1-carbonyl)phenyl)-3-(imidazo[1,2-a]pyridin-5-yl)-1-methyl-4-(trifluoromethyl)-1H-pyrazole-5-carboxamide (I-241) as an off white solid (7 mg, 11%). (ES, m/z): [M+H]⁺=519.4; ¹H NMR (400 MHz, DMSO-d₆) δ 11.54 (s, 1H), 7.94 (d, J=1.6 Hz, 1H), 7.91 (s, 1H), 7.76-7.73 (m, 1H), 7.68 (d, J=1.3 Hz, 1H), 7.64 (dd, J=1.8, 8.4 Hz, 1H), 7.48 (d, J=8.4 Hz, 1H), 7.40 (dd, J=7.0, 9.1 Hz, 1H), 7.05 (d, J=6.6 Hz, 1H), 6.63 (s, 1H), 4.52 (m, 1H), 4.24 (dd, J=7.5, 10.0 Hz, 1H), 4.08-4.04 (m, 4H), 3.77 (dd, J=4.7, 10.7 Hz, 1H), 3.72 (dd, J=4.4, 9.2 Hz, 1H).

### Example 59—Synthesis of 3-(2-aminobenzo[d]thiazol-7-yl)-N-(5-chloro-6-(2H-1,2,3-triazol-2-yl) pyridin-3-yl)-1-methyl-4-(trifluoromethyl)-1H-pyrazole-5-carboxamide (I-299)

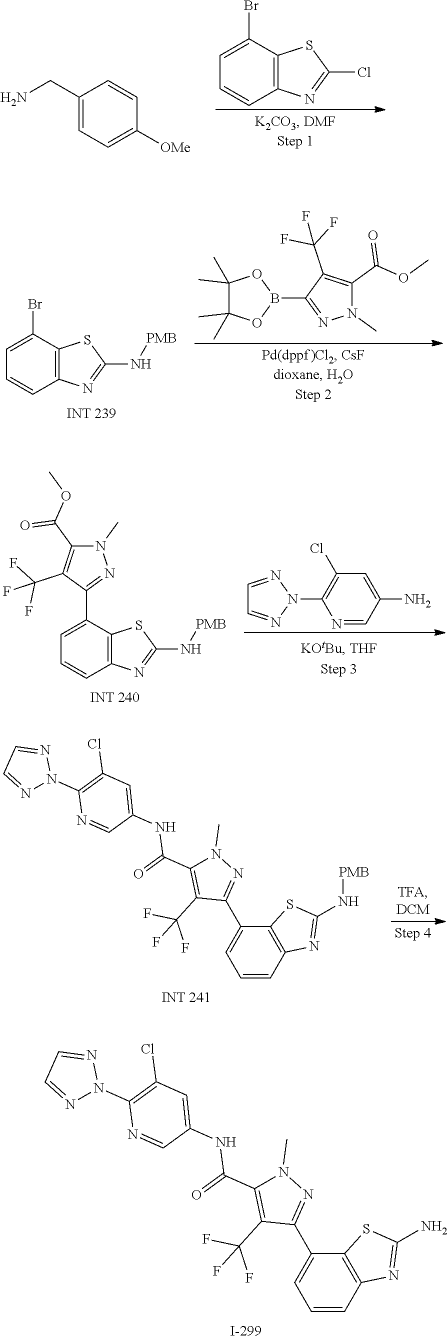

Step 1: Synthesis of 7-bromo-N-(4-methoxybenzyl)benzo[d]thiazol-2-amine (INT 239): 4-Methoxybenzylamine (0.47 mL, 3.62 mmol, 3.00 eq) was added to a stirred mixture of 7-bromo-2-chloro-1,3-benzothiazole (300 mg, 1.21 mmol, 1.00 eq) and K₂CO₃ (333 mg, 2.41 mmol, 2.00 eq) in DMF (5 mL), and the reaction was heated to 100° C. for 16 h. The residue was diluted with EtOAc and NaHCO₃, the layers were separated and the organic layer was washed with NaHCO₃ and brine. The organics were dried (MgSO₄) and concentrated. The crude residue was purified by column chromatography eluting with 0-100% EtOAc in cyclohexane to give INT 239 as a white solid (330 mg, 0.95 mmol, 78%). (ES, m/z): [M+H]⁺=348.9/350.9; ¹H NMR (400 MHz, DMSOd₆) δ 8.66 (dd, J=5.7, 5.7 Hz, 1H), 7.38 (dd, J=1.7, 7.2 Hz, 1H), 7.31 (d, J=8.7 Hz, 2H), 7.23-7.16 (m, 2H), 6.92 (d, J=8.7 Hz, 2H), 4.51 (d, J=5.6 Hz, 2H), 3.74 (s, 3H).

Step 2: Synthesis of methyl 3-(2-((4-methoxybenzyl)amino)benzo[d]thiazol-7-yl)-1-methyl-4-(trifluoromethyl)-1H-pyrazole-5-carboxylate (INT 240): The title compound was synthesized from INT 239 based on the procedures described in Example 10 and isolated by column chromatography with 0-100% EtOAc in cyclohexane to give INT 240 as a off white solid (120 mg, 0.25 mmol, 56%). (ES, m/z): [M+H]⁺=477.2; ¹H NMR (400 MHz, CDCl₃) 7.58 (1H, d, J=7.7 Hz), 7.38-7.29 (3H, m), 7.20 (1H, d, J=7.7 Hz), 6.89 (2H, d, J=8.7 Hz), 5.42 (1H, s), 4.59-4.57 (2H, m), 4.18 (3H, s), 4.00 (3H, s), 3.80 (3H, s).

Step 3: Synthesis of N-(5-chloro-6-(2H-1,2,3-triazol-2-yl) pyridin-3-yl)-3-(2-((4-methoxybenzyl)amino)benzo[d]thiazol-7-yl)-1-methyl-4-(trifluoromethyl)-1H-pyrazole-5-carboxamide (INT 241): The title compound was synthesized from INT 240 based on procedures described in Example 6 and isolated by column chromatography eluting with 0-100% EtOAc in cyclohexane to give INT 241 as a pale yellow solid (45 mg, 0.07 mmol, 55%). (ES, m/z): [M+H]⁺ 640.1.

Step 4: Synthesis of 3-(2-aminobenzo[d]thiazol-7-yl)-N-(5-chloro-6-(2H-1,2,3-triazol-2-yl) pyridin-3-yl)-1-methyl-4-(trifluoromethyl)-1H-pyrazole-5-carboxamide (I-299): TFA (1 mL) was added to a stirred mixture of N-[5-chloro-6-(triazol-2-yl)-3-pyridyl]-5-[2-[(4-methoxyphenyl)methylamino]-1,3-benzothiazol-7-yl]-2-methyl-4-(trifluoromethyl) pyrazole-3-carboxamide (45 mg, 0.0703 mmol, 1.00 eq) and anisole (0.023 mL, 0.211 mmol, 3.00 eq) in DCM (1 mL). The reaction heated at 60° C. for 16 h. The reaction was concentrated to dryness and the residue was purified by reverse phase HPLC to give 3-(2-aminobenzo[d]thiazol-7-yl)-N-(5-chloro-6-(2H-1,2,3-triazol-2-yl) pyridin-3-yl)-1-methyl-4-(trifluoromethyl)-1H-pyrazole-5-carboxamide (I-299) as an off white solid (8.25 mg, 0.16 mmol, 22%). (ES, m/z): [M+H]⁺=520.2; ¹H NMR (400 MHz, DMSO-d₆) δ 11.96 (s, 1H), 8.80 (s, 1H), 8.63 (d, J=2.3 Hz, 1H), 8.20 (s, 2H), 7.51 (s, 2H), 7.44-7.41 (m, 1H), 7.36 (dd, J=7.7, 7.7 Hz, 1H), 7.22 (d, J=7.6 Hz, 1H), 4.04 (s, 3H).

### Example 60—N-(2-cyclopropylpyridin-4-yl)-3-(imidazo[1,2-a]pyridin-5-yl)-1-methyl-4-(trifluoromethyl)-1H-pyrazole-5-carboxamide (I-227)

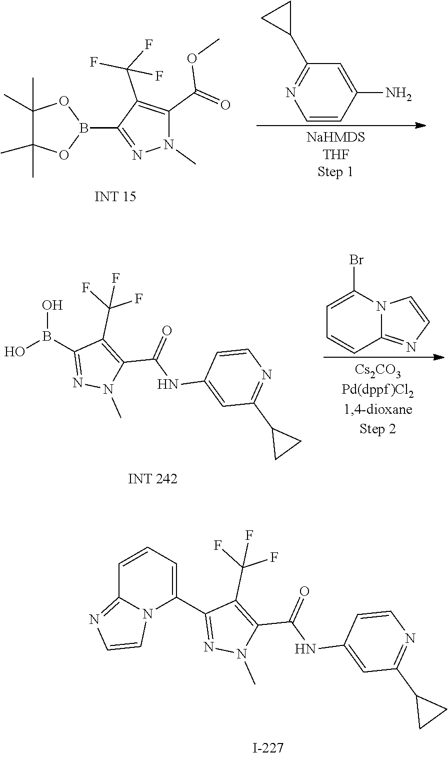 [SMILES: COC(=O)c1c(C(F)(F)F)c(B2OC(C)(C)C(C)(C)O2)nn1C ; Nc1ccnc(C2CC2)c1 ; Cn1nc(B(O)O)c(C(F)(F)F)c1C(=O)Nc1ccnc(C2CC2)c1 ; Brc1cccc2nccn12 ; Cn1nc(-c2cccc3nccn23)c(C(F)(F)F)c1C(=O)Nc1ccnc(C2CC2)c1]

Step 1: Synthesis of (5-((2-cyclopropylpyridin-4-yl) carbamoyl)-1-methyl-4-(trifluoromethyl)-1H-pyrazol-3-yl) boronic acid (INT 242): A 1 M sodium bis(trimethylsilyl)amide solution in THF (1023 μL, 1.02 mmol, 2.01 eq) was added to a solution of methyl 1-methyl-3-(4,4,5,5-tetramethyl-1,3,2-dioxaborolan-2-yl)-4-(trifluoromethyl)-1H-pyrazole-5-carboxylate (INT 15, 170 mg, 0.509 mmol, 1.00 eq) and 2-cyclopropylpyridin-4-amine (68 mg, 0.507 mmol, 0.996 eq) in tetrahydrofuran (2 mL) at 0° C. The reaction was stirred for 1 hour then quenched with saturated aqueous NH₄Cl (1 mL), extracted with ethyl acetate (2× 10 mL), dried (MgSO₄) and concentrated under reduced pressure to give INT 242 (275 mg) as an oily solid which was used without further purification in the next reaction.

Step 2: N-(2-cyclopropylpyridin-4-yl)-3-(imidazo[1,2-a]pyridin-5-yl)-1-methyl-4-(trifluoromethyl)-1H-pyrazole-5-carboxamide (I-227): Pd(dppf)Cl₂ (37 mg, 0.0500 mmol, 0.100 eq) was added to a stirred degassed mixture of (5-((2-cyclopropylpyridin-4-yl) carbamoyl)-1-methyl-4-(trifluoromethyl)-1H-pyrazol-3-yl) boronic acid (177 mg, 0.500 mmol, 1.00 eq), Cs₂CO₃ (489 mg, 1.50 mmol, 3.00 eq) and 5-bromoimidazo[1,2-a]pyridine (148 mg, 0.750 mmol, 1.50 eq) in dioxane (4 mL). The reaction mixture was heated to reflux for 1 h. The reaction was cooled, filtered through celite and the filter cake washed with THF (5 mL). The solvent was evaporated from the filtrate, and water (2 mL) was added. The mixture was extracted with ethyl acetate (2×10 mL). The combined organic extracts were dried (MgSO₄) and concentrated under reduced pressure to give a brown oil. The crude material was purified by column chromatography eluting with 20-100% EtOAc in cyclohexane followed by 0-30% EtOH in EtOAc to give partially purified product which was further purified by preparative HPLC to afford N-(2-cyclopropylpyridin-4-yl)-3-(imidazo[1,2-a]pyridin-5-yl)-1-methyl-4-(trifluoromethyl)-1H-pyrazole-5-carboxamide (I-227, 14.8 mg, 0.035 mmol, 7%) as an off-white solid. (ES, m/z): [M+H]⁺ 427.2, ¹H NMR (400 MHz, DMSO) δ 11.45 (s, 1H), 8.38 (d, J=5.6 Hz, 1H), 7.90 (s, 1H), 7.75 (td, J=0.9, 9.1 Hz, 1H), 7.68 (d, J=1.3 Hz, 1H), 7.60 (s, 1H), 7.42-7.37 (m, 2H), 7.06 (d, J=6.8 Hz, 1H), 4.06 (s, 3H), 2.13-2.07 (m, 1H), 0.97-0.91 (m, 4H).

### Example 61—Synthesis of N-(1-methyl-3-(1-methyl-1H-benzo[d]imidazol-7-yl)-4-(trifluoromethyl)-1H-pyrazol-5-yl)-2-(trifluoromethyl)isonicotinamide (I-221)

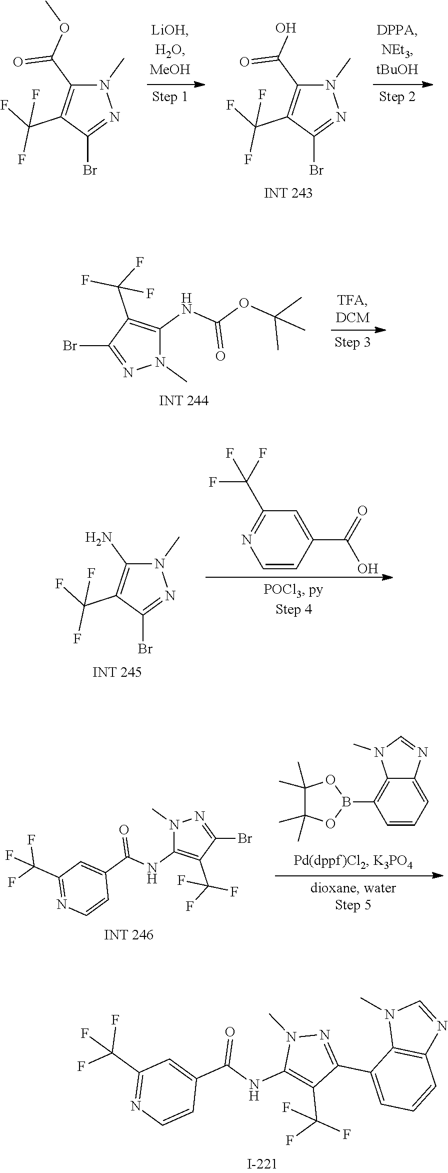

Step 1: Synthesis of 3-bromo-1-methyl-4-(trifluoromethyl)-1H-pyrazole-5-carboxylic acid (INT 243): LiOH·H₂O (92 mg, 2.19 mmol, 1.10 eq) was added to a stirred mixture of ethyl 5-bromo-2-methyl-4-(trifluoromethyl) pyrazole-3-carboxylate (600 mg, 1.99 mmol, 1.00 eq) in MeOH (15.00 mL) and water (7.5 mL). The mixture was stirred at rt for 16 h. The reaction was concentrated to dryness, re-dissolved in H₂O and the pH adjusted to 1 using 2 M aqueous HCl. The suspension was extracted with EtOAc and the organics were combined, dried (MgSO₄) and concentrated to give INT 243 as an off-white solid (500 mg, 1.83 mmol, 91%). (ES, m/z): [M+H]⁺=271.2/273.2; ¹H NMR (400 MHz, DMSO-d₆) δ 4.03 (s, 3H).

Step 2: Synthesis of tert-butyl (3-bromo-1-methyl-4-(trifluoromethyl)-1H-pyrazol-5-yl) carbamate (INT 244): Diphenyl phosphoryl azide (0.47 mL, 2.20 mmol, 1.20 eq) was added dropwise to a stirred solution of 5-bromo-2-methyl-4-(trifluoromethyl) pyrazole-3-carboxylic acid (500 mg, 1.83 mmol, 1.00 eq) in t-BuOH (5 mL). The mixture was then heated to 100° C. and stirred for 16 h. The reaction was concentrated to dryness, diluted with EtOAc and washed with saturated aqueous NaHCO₃ and brine. The organic phase was dried (MgSO₄) and concentrated. The crude residue was taken forward to the next step without further purification (INT 244, 500 mg, ˜60% pure). (ES, m/z): [M+H]⁺ 342.0/344.0

Step 3: Synthesis of 3-bromo-1-methyl-4-(trifluoromethyl)-1H-pyrazol-5-amine (INT 245): TFA (0.67 mL, 8.72 mmol, 10.0 eq) was added to a stirred solution of tert-butyl N-[5-bromo-2-methyl-4-(trifluoromethyl) pyrazol-3-yl]carbamate (60%, 500 mg, 0.872 mmol, 1.00 eq) in DCM (10 mL) and the mixture was stirred at rt for 16 h. The mixture was concentrated under reduced pressure to give the title compound which was carried forward to the next step without further purification (INT 245, 450 mg, ˜45% purity). (ES, m/z): [M+H]⁺ 242.0/244.0.

Step 4: Synthesis of N-(3-bromo-1-methyl-4-(trifluoromethyl)-1H-pyrazol-5-yl)-2-(trifluoromethyl)isonicotinamide (INT 246): POCl₃ (0.12 mL, 1.26 mmol, 2.00 eq) was added dropwise to a stirred solution of 5-bromo-2-methyl-4-(trifluoromethyl) pyrazol-3-amine (45%, 375 mg, 0.691 mmol, 1.10 eq) and 2-(trifluoromethyl)pyridine-4-carboxylic acid (120 mg, 0.628 mmol, 1.00 eq) in pyridine (5 mL) and the mixture was stirred at rt for 3 h. The reaction was diluted with EtOAc and washed with saturated aqueous NaHCO₃ and brine. The organics were dried (MgSO₄) and concentrated. The crude residue was purified by column chromatography eluting with 0-100% EtOAc in cyclohexane to give INT 246 as an off-white solid (100 mg, 0.24 mmol, 38%). (ES, m/z): [M+H]⁺ 415.4/417.4, ¹H NMR (400 MHz, CDCl₃) δ 9.01 (d, J=5.0 Hz, 1H), 8.12 (s, 1H), 7.92 (d, J=4.5 Hz, 1H), 7.78 (s, 1H), 3.82 (s, 3H).

Step 5: Synthesis of N-(1-methyl-3-(1-methyl-1H-benzo[d]imidazol-7-yl)-4-(trifluoromethyl)-1H-pyrazol-5-yl)-2-(trifluoromethyl)isonicotinamide (I-221): The title compound was synthesised from N-(3-bromo-1-methyl-4-(trifluoromethyl)-1H-pyrazol-5-yl)-2-(trifluoromethyl)isonicotinamide using the procedures described in Example 2. Purification was achieved by reverse phase HPLC to give N-(1-methyl-3-(1-methyl-1H-benzo[d]imidazol-7-yl)-4-(trifluoromethyl)-1H-pyrazol-5-yl)-2-(trifluoromethyl)isonicotinamide (I-221) as an off-white solid (2.38 mg, 0.0051 mmol, 4%). (ES, m/z): [M+H]⁺=469.6; ¹H NMR (400 MHz, DMSO-d₆) δ 11.31-11.29 (m, 1H), 9.06 (d, J=2.0 Hz, 1H), 8.43 (s, 1H), 8.24 (d, J=1.5 Hz, 1H), 8.19 (d, J=2.0 Hz, 1H), 7.78-7.75 (m, 1H), 7.29-7.26 (m, 1H), 7.23-7.20 (m, 1H), 3.85 (s, 3H), 3.46-3.41 (m, 3H).

### Example 62-Synthesis of 3-(3,4-dihydroquinolin-1 (2H)-yl)-1-methyl-4-(trifluoromethyl)-N-(2-(trifluoromethyl)pyridin-4-yl)-1H-pyrazole-5-carboxamide (I-85)

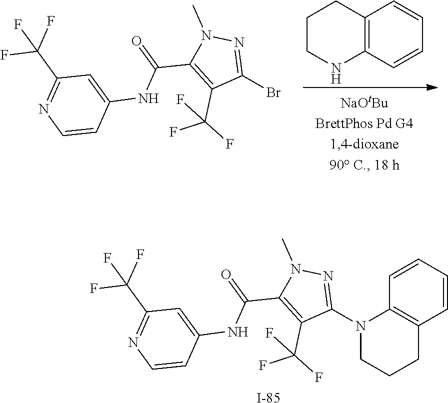

2 M NaO^{t}Bu solution in THF (0.54 mL, 1.08 mmol, 2.60 eq) was added dropwise to a degassed solution of 5-bromo-2-methyl-4-(trifluoromethyl)-N-[2-(trifluoromethyl)-4-pyridyl]pyrazole-3-carboxamide (174 mg, 0.417 mmol, 1.00 eq) and 1,2,3,4-tetrahydroquinoline (0.068 mL, 0.542 mmol, 1.30 eq) in anhydrous 1,4-dioxane (5.00 mL). BrettPhos Pd G4 (38 mg, 0.0417 mmol, 0.1000 eq) was then added to the reaction mixture. The reaction mixture was heated at 90° C. for 18 h. The reaction mixture was cooled, concentrated and the crude residue purified by preparative HPLC to afford 3-(3,4-dihydroquinolin-1 (2H)-yl)-1-methyl-4-(trifluoromethyl)-N-(2-(trifluoromethyl)pyridin-4-yl)-1H-pyrazole-5-carboxamide (I-85, 0.49 mg, 0.001 mmol, 1%) as an off-white solid. (ES, m/z): [M+H]⁺ 470.0, ¹H NMR (400 MHz, DMSO-d₆) δ 11.84 (s, 1H), 8.74 (s, 1H), 8.18 (s, 1H), 7.87 (s, 1H), 6.93 (dd, J=7.7, 7.7 Hz, 2H), 6.53-6.48 (m, 2H), 3.96 (s, 3H), 3.24-3.23 (m, 2H), 2.77-2.73 (m, 2H), 1.83-1.77 (m, 2H).

The following compound in Table 22 was synthesized following a similar procedure to that described above.

TABLE 22

| Compound | | Starting | |
|---|---|---|---|
| No. | Structure | Material | Analytical Data |
| I-209 | 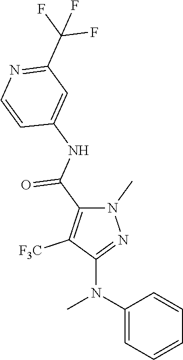 [SMILES: CN(c1ccccc1)c1nn(C)c(C(=O)Nc2ccnc(C(F)(F)F)c2)c1C(F)(F)F] | N- methylan- iline | (ES, m/z): [M + H]⁺ 444.2, ¹H NMR (400 MHz, DMSO-d₆) δ 11.79 (s, 1H), 8.71 (s, 1H), 8.14 (s, 1H), 7.85 (s, 1H), 7.22 (dd, J = 7.3, 8.6 Hz, 2H), 6.83 (dd, J = 7.3, 7.3 Hz, 1H), 6.76 (d, J = 8.1 Hz, 2H), 3.91 (s, 3H), 3.20 (s, 3H). |

### Example 63—Synthesis of ethyl 1-methyl-3-(phenylamino)-4-(trifluoromethyl)-1H-pyrazole-5-carboxylate (INT 247)

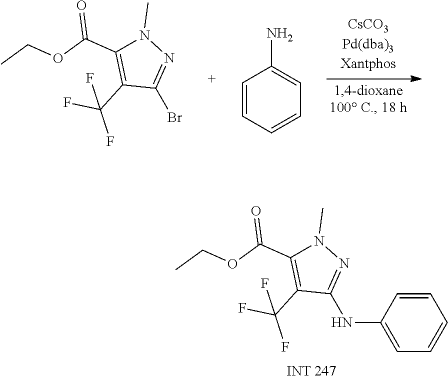 [SMILES: CCOC(=O)c1c(C(F)(F)F)c(Br)nn1C ; Nc1ccccc1 ; CCOC(=O)c1c(C(F)(F)F)c(Nc2ccccc2)nn1C]

Aniline (15 mg, 0.166 mmol, 1.00 eq), tris(dibenzylideneacetone) dipalladium (0) (7.6 mg, 8.30 μmol, 0.0500 eq) and Xantphos (10 mg, 0.0166 mmol, 0.100 eq) were added to a degassed reaction mixture of ethyl 5-bromo-2-methyl-4-(trifluoromethyl) pyrazole-3-carboxylate (50 mg, 0.166 mmol, 1.00 eq) and Cs₂CO₃ (162 mg, 0.498 mmol, 3.00 eq) in anhydrous 1,4-dioxane (2.00 mL). The reaction was then stirred at 100° C. for 18 h. The reaction mixture was cooled, diluted with EtOAc and washed with water. The organic phase was dried (MgSO₄) and concentrated. The crude product was purified by column chromatography eluting with 0-50% EtOAc in cyclohexane to afford INT 247 (42 mg, 0.134 mmol, 81%) as a yellow gum. (ES, m/z): [M+H]+ 314.0.

### Example 64—Synthesis of 5-bromo-8-fluoroimidazo[1,2-a]pyridine (INT 248)

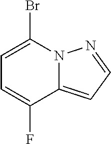 [SMILES: Fc1ccc(Br)n2nccc12]

Hydrogen bromide solution (48%, 0.15 mL, 1.31 mmol, 1.00 eq) was added to a stirred solution of 6-bromo-3-fluoro-pyridin-2-amine (250 mg, 1.31 mmol, 1.00 eq) and bromoacetaldehyde diethyl acetal (0.30 mL, 1.96 mmol, 1.50 eq) in ethanol (20 mL). The resulting mixture was stirred for 1 h at room temperature. The reaction mixture was concentrated under reduced pressure to dryness. The residue was partitioned between EtOAc and saturated NaHCO₃ and the phases separated. The organic extract was dried (MgSO₄) and concentrated under reduced pressure to afford INT 248 (188 mg, 67%) as a brown oil. (ES, m/z): [M+H]+ 216.9.

The following compounds in Table 23 were synthesized following a similar procedure to that described above.

TABLE 23

| Intermedi- | | Starting | |
|---|---|---|---|
| ate | Structure | Material | Analytical Data |
| INT 236 | 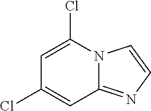 [SMILES: Clc1cc(Cl)n2ccnc2c1] | 4,6- dichloropyri- din-2- amine | (ES, m/z): [M + H]+ 186.9, ¹H NMR (400 MHz, CDCl₃) δ 7.75 (s, 1H), 7.71 (s, 1H), 7.62 (d, J = 1.4 Hz, 1H), 6.94 (d, J = 1.8 Hz, 1H). |
| INT 237 | 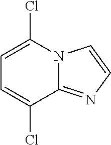 [SMILES: Clc1ccc(Cl)n2ccnc12] | 3,6- dichloropyri- din-2- amine | (ES, m/z): [M + H]+ 186.9, ¹H NMR (400 MHz, CDCl₃) δ 7.84 (d, J = 1.1 Hz, 1H), 7.78-7.77 (m, 1H), 7.28 (d, J = 7.9 Hz, 1H), 6.88- 6.85 (m, 1H). |
| INT 238 | 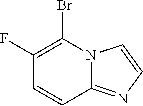 [SMILES: Fc1ccc2nccn2c1Br] | 6-bromo-5- fluoropyridin- 2-amine | (ES, m/z): [M + H]+ 214.9/216.9, ¹H NMR (400 MHz, CDCl₃) δ 7.84 (s, 1H), 7.76 (s, 1H), 7.61 (dd, J = 4.7, 9.7 Hz, 1H), 7.19 (dd, J = 7.7, 9.7 Hz, 1H). |

### Example 65—Synthesis of 7-bromo-6-methoxybenzo[d]thiazole (INT 250)

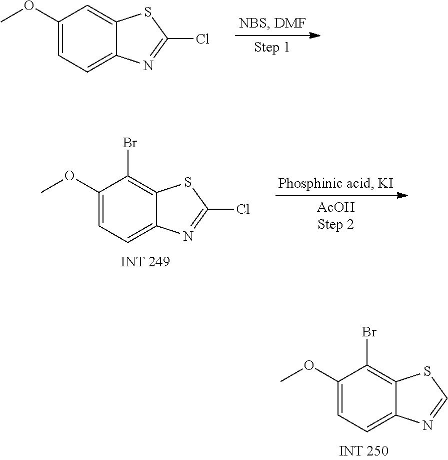

Step 1: Synthesis of 7-bromo-2-chloro-6-methoxybenzo[d]thiazole (INT 249): N-bromosuccinimide (2139 mg, 12.0 mmol, 1.20 eq) was added to a stirred solution of 2-chloro-6-methoxybenzothiazole (2000 mg, 10.0 mmol, 1.00 eq) in DMF (100 mL) and the reaction mixture was stirred at rt for 72 h. The mixture was concentrated under reduced pressure and the crude residue was suspended between EtOAc and water. The phases were separated and the organic extract was further washed with water, NaHCO₃ and brine. The organic phase was dried (MgSO₄), concentrated and the crude residue was purified by column chromatography eluting with 0-100% EtOAc in cyclohexane to give the INT 249 as an off white solid (1.9 g, 6.82 mmol, 68%). (ES, m/z): [M+H]+ 277.8/279.8/281.8, ¹H NMR (400 MHz, DMSO-d₆) δ 7.99 (d, J=8.9 Hz, 1H), 7.41 (d, J=9.0 Hz, 1H), 3.96 (s, 3H).

Step 2: Synthesis of 7-bromo-6-methoxybenzo[d]thiazole (INT 250): Phosphinic acid (1.9 mL, 35.9 mmol, 10.0 eq) was added to a stirred mixture of 7-bromo-2-chloro-6-methoxy-1,3-benzothiazole (1000 mg, 3.59 mmol, 1.00 eq) and KI (1788 mg, 10.8 mmol, 3.00 eq) in AcOH (15 mL), and the reaction was then heated at 80° C. for 1 h. The reaction was cooled to rt and poured onto ice. The reaction was then adjusted to neutral pH using a 2 M aqueous NaOH solution. The mixture was then extracted with DCM and the organic extracts combined, dried (MgSO₄) and concentrated under reduced pressure to give the 7-bromo-6-methoxybenzo[d]thiazole (INT 250) as a white solid (800 mg, 3.2 mmol, 91%). (ES, m/z): [M+H]⁺=243.9/245.9, ¹H NMR (400 MHz, CDCl₃) δ 8.88 (s, 1H), 8.03 (d, J=8.9 Hz, 1H), 7.17 (d, J=8.9 Hz, 1H), 4.00 (s, 3H).

### Example 66—Synthesis of 7-bromo-N-methylbenzo[d]thiazol-2-amine (INT 251)

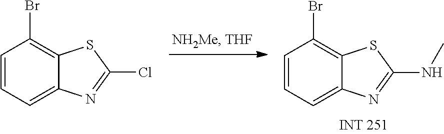

A 2 M solution of methylamine in THF (9.1 mL, 18.1 mmol, 15.0 eq) was added to a tube containing 7-bromo-2-chloro-1,3-benzothiazole (300 mg, 1.21 mmol, 1.00 eq), the reaction tube was sealed, and the reaction was heated to 60° C. for 16 h. The mixture was cooled to rt and concentrated to dryness. The residue was purified by column chromatography eluting with 0-80% EtOAc in cyclohexane to give INT 251 as a white solid (240 mg, 0.99 mmol, 81%). (ES, m/z): [M+H]+ 242.9/244.9, ¹H NMR (400 MHz, DMSO-d₆) δ 8.20-8.17 (m, 1H), 7.38 (dd, J=2.0, 6.9 Hz, 1H), 7.23-7.16 (m, 2H), 2.94 (d, J=4.6 Hz, 3H).

### Example 67—Synthesis of 7-bromo-N,N-dimethylbenzo[d]thiazol-2-amine (INT 252)

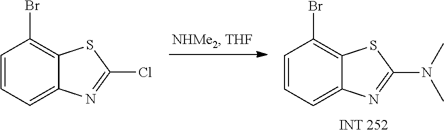

A 2 M solution of dimethylamine in THF (9.0 mL, 18.0 mmol, 14.9 eq) was added to a tube containing 7-bromo-2-chloro-1,3-benzothiazole (300 mg, 1.21 mmol, 1.00 eq), the tube was sealed, and the reaction was heated to 60° C. for 16 h. The reaction was cooled to rt and concentrated to dryness. The residue was purified by column chromatography eluting with 0-80% EtOAc in cyclohexane to give INT 252 as a white solid (280 mg, 1.08 mmol, 90%). (ES, m/z): [M+H]+ 256.9/258.9; 1H NMR (400 MHz, DMSO-d₆) δ 7.43 (dd, J=3.7, 5.3 Hz, 1H), 7.24-7.22 (m, 2H), 3.16 (s, 6H).

### Example 68—Synthesis of 7-bromo-2-methoxybenzo[d]thiazole (INT 253)

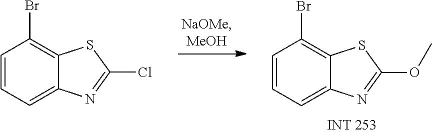

Sodium methoxide (652 mg, 12.1 mmol, 10.0 eq) was added to a stirred solution of 7-bromo-2-chloro-1,3-benzothiazole (300 mg, 1.21 mmol, 1.00 eq) in MeOH (10 mL). The reaction mixture was stirred at 60° C. for 16 h. The reaction was cooled to rt and concentrated. The residue was diluted with EtOAc and NaHCO₃, the layers were separated, and the organic extract washed with NaHCO₃ and brine. The organic phase was dried (MgSO₄) and concentrated. The crude residue was purified by column chromatography eluting with 0-100% EtOAc in cyclohexane to give INT 253 as a white solid (200 mg, 0.82 mmol, 67%). (ES, m/z): [M+H]+ 243.9/245.9; ¹H NMR (400 MHz, CDCl₃) δ 7.62 (d, J=8.0 Hz, 1H), 7.36 (d, J=7.9 Hz, 1H), 7.27-7.23 (m, 1H), 4.20 (s, 3H).

### Example 69—Synthesis of 7-bromo-6-fluoro-1,3-benzothiazole (INT 257)

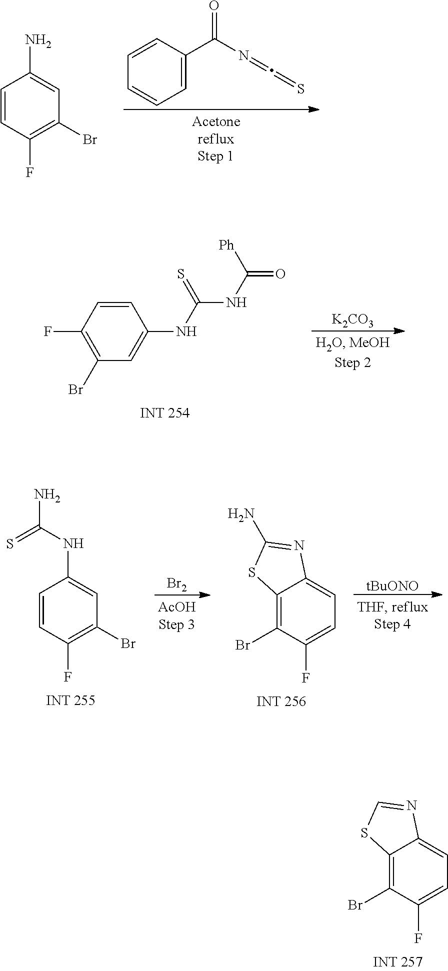

Step 1: Synthesis of N-((3-bromo-4-fluorophenyl)carbamothioyl)benzamide (INT 254): Benzoyl isothiocyanate (1.2 mL, 8.97 mmol, 1.20 eq) was added to a solution of 3-bromo-4-fluoro-aniline (1420 mg, 7.47 mmol, 1.00 eq) in acetone (40.00 mL) and the mixture stirred at 60° C. for 4 h. The solvent was removed under reduced pressure and the crude solid triturated with cyclohexane. The suspension was filtered and the solid dried under vacuum to yield INT 254 (2558 mg, 7.24 mmol, 97%) as a beige solid. (ES, m/z): [M+H]⁺=352.9/354.9; 1H NMR (400 MHz, DMSO-d₆) δ 12.49 (s, 1H), 11.69 (s, 1H), 8.11 (dd, J=6.2, 2.3 Hz, 1H), 8.01-7.95 (m, 2H), 7.71-7.61 (m, 2H), 7.55 (t, J=7.7 Hz, 2H), 7.45 (t, J=8.7 Hz, 1H).

Step 2: Synthesis of 1-(3-bromo-4-fluorophenyl)thiourea (INT 255): A solution of K₂CO₃ (2803 mg, 20.3 mmol, 2.80 eq) in water (10 mL) was added to a stirred solution of N-[(3-bromo-4-fluoro-phenyl)carbamothioyl]benzamide (2558 mg, 7.24 mmol, 1.00 eq) in MeOH (20.00 mL). The resulting reaction mixture was heated to 80° C. for 1 h. The reaction was allowed to cool down to rt and the solvents were removed under reduced pressure. The residue was treated with water (10 mL), filtered and the solid dried under reduced pressure to yield INT 255 (1610 mg, 6.46 mmol, 89%) as a beige solid which was used in the next step without further purification. (ES, m/z): [M+H]⁺=248.9/250.9; ¹H NMR (400 MHz, DMSO-d₆) δ 9.57 (br s, 1H), 7.86 (dd, J=6.2, 2.2 Hz, 1H), 7.68 (br s, 2H), 7.40-7.25 (m, 2H).

Step 3: Synthesis of 7-bromo-6-fluorobenzo[d]thiazol-2-amine (INT 256): Bromine (0.36 mL, 7.11 mmol, 1.10 eq) was added dropwise to a stirred solution of (3-bromo-4-fluoro-phenyl)thiourea (1610 mg, 6.46 mmol, 1.00 eq) in acetic acid (20 mL) at 10° C. under a nitrogen atmosphere. The resulting mixture was allowed to warm to rt and stirred for 15 minutes, then it was heated to 80° C. for 2 h. The mixture was cooled to rt and poured into water (50 mL). The resulting mixture was extracted with EtOAc (4×20 mL). The combined organics were filtered, washed with brine (50 mL), dried (MgSO₄) and the solvents removed under reduced pressure. The residue was purified by SFC to give INT 256 as an off white solid (454 mg, 1.84 mmol, 28%). (ES, m/z): [M+H]⁺ 247.0/249.0, ¹H NMR (400 MHz, DMSO-d₆) δ 7.70 (br s, 2H), 7.31 (dd, J=4.3, 8.7 Hz, 1H), 7.22 (dd, J=8.7, 9.3 Hz, 1H).

Step 4: Synthesis of 7-bromo-6-fluorobenzo[d]thiazole (INT 257): Tert-butyl nitrite (90%, 0.24 mL, 1.82 mmol, 3.00 eq) was added to a solution of 7-bromo-6-fluoro-1,3-benzothiazol-2-amine (150 mg, 0.607 mmol, 1.00 eq) in THF (20 mL) which was then heated at 70° C. for 16 h. The reaction mixture was concentrated, and the crude product was purified by silica gel column chromatography eluting with 0-50% EtOAc in cyclohexane to give INT 257 as an off white solid (130 mg, 0.560 mmol, 92%). (ES, m/z): [M+H]⁺ 232, ¹H NMR (400 MHz, DMSO-d₆) δ 9.49 (s, 1H), 8.16 (dd, J=4.4, 8.9 Hz, 1H), 7.61 (t, J=9.0 Hz, 1H).

### Example 70—Synthesis of Additional Compounds

Compounds in the table below were prepared using procedures based on those described herein above.

| Compound | |
|---|---|
| No. | Chemical Structure |
| I-123 | 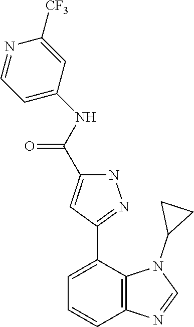 [SMILES: O=C(Nc1ccnc(C(F)(F)F)c1)c1cc(-c2cccc3ncn(C4CC4)c23)n[nH]1] |

### Example 71—MALT1 Inhibition Assay

Compounds were tested for ability to bind to MALT1. Experimental procedures and results are provided below.

### Part I—Experimental Procedures

Inhibition of MALT1 activity by the presence of small molecules was evaluated using MALT-1 Fluorogenic Peptide Cleavage Assay. The assay utilizes a quenched AMC-labelled peptide that contains the MALT-1 recognition sequence and cleavage site (LRSR). MALT-1 mediated cleavage of the peptide relieves the quenching and leads to an increase in fluorescence at excitation (342 nm) and emission (441 nm).

The following reagents were obtained commercially and used to prepare standard reagent formulations as further described below: N-2-hydroxyethylpiperazine-N-2-ethane sulfonic acid (HEPES) (1 M, pH 7.5, stored at 4° C.), sodium citrate (stored at RT), tris(2-carboxyethyl) phosphine hydrochloride (T-CEP) (500 mM, stored at −20° C.), ethylenediaminetetraacetic acid (EDTA) (500 mM, stored at RT), dimethylsulfoxide (DMSO, stored at RT), 3-[(3-cholamidopropyl)dimethylammonio]-1-propane sulphate (CHAPS) (stored at RT), dimethylsulfoxide (DMSO), DMSO (Fisher Scientific, stored at RT), Avi-tagged FL MALT-1 (Pharmaron, stored at −70° C.), Ac-LRSR-AMC Peptide (SM Biochemicals, stored at −20° C.), and ultrapure water (MILLI-QR).

The standard reagent formulations used in this assay were prepared and stored as follows. A 1.1 M solution of sodium citrate (161.3 g, 500 mL) was prepared and stored at room temperature. A 10% (w/v) CHAPS solution (2.0 g, 20 mL) was prepared and stored at 4° C. A 500 mM HEPES solution having pH of 6.89 was prepared from 200 mL of a 1 M HEPES solution having a pH of 7.5 using concentrated hydrochloric acid and brought to a final volume of 400 mL with MILLI-QR H₂O. The substrate, 10 mM Ac-LRSR-AMC Peptide, was prepared (10 mg, 1.370 mL DMSO) and stored at −20° C.

Compounds were plated to provide a 2% DMSO final concentration using a ProxiPlate-384 Plus F Black 384-shallow well microplate. The assay-ready plates were equilibrated to room temperature. A reaction buffer (30 mL total volume) was prepared by combining HEPES (pH 6.89, 25 mM, 1.5 mL), sodium citrate (660 mM, 18.0 mL), T-CEP (1 mM, 0.06 mL), EDTA (0.1 mM, 0.06 mL), CHAPS (0.05%, 0.15 mL), DMSO (2%, 0.6 mL), and MILLI-QR H₂O (10.23 mL; 9.63 mL when backfilled to 2% DMSO) followed by thorough mixing. DMSO was added only when compound plates had not been DMSO-backfilled to 2%.

MALT-1 was thawed and kept on ice. Peptide substrate was thawed on the bench under ambient conditions. A MALT-1 enzyme working stock was prepared from Avi-tagged FL MALT-1 (40 nM in a prepared reagent volume of 16.5 μL) and the reaction buffer (13.0 mL). MALT1 working stock (5 μL) was added to each well of the microplate. MALT-1 was pre-incubated with compounds for 30 minutes at room temperature.

Two substrate working stocks (Km and 10×Km) were prepared. The 1×Km substrate was prepared from Ac-LRSR-AMC Peptide (50 μM in a prepared reagent volume of 35.0 μL) and the reaction buffer (6.965 mL). The 10×Km substrate was prepared from Ac-LRSR-AMC Peptide (280 μM in a prepared reagent volume of 196.0 μL) and the reaction buffer (6.804 mL). The reaction was initiated by the addition of substrate working stock (5 μL, 50 μM) to Km plates and the addition of substrate working stock (5 μL 280 μM) to 10×Km plates. The plates were covered and incubated on the bench at room temperature for 90 minutes.

Fluorescence intensity was determined using a CLARIOstar microplate reader (BMG LABTECH) using the optimised AMC mode. Before taking readings, a 70% gain was applied on the neutral control well. Compound data were normalised to % Inhibition and used to plot sigmoidal concentration response curves to yield various parameters including IC₅₀.

### Part II—Results

The results of the MALT1 binding assay are reported in Table 24. Compounds with an IC₅₀ less than 0.5 μM are designated as “A”. Compounds with an IC₅₀ greater than 0.5 μM and less than or equal to 2.5 μM are designated as “B”. Compounds with a IC₅₀ greater than 2.5 UM and less than or equal to 10 μM are designated as “C”. Compounds with an IC₅₀ greater than 10 μM are designated as “D”.

TABLE 24

| Compound No. | MALT1 IC₅₀ |
|---|---|
| I-1 | A |
| I-2 | C |
| I-3 | C |
| I-4 | A |
| I-5 | C |
| I-6 | A |
| I-7 | B |
| I-8 | C |
| I-9 | A |
| I-10 | B |
| I-11 | A |
| I-12 | B |
| I-13 | B |
| I-14 | A |
| I-15 | D |
| I-16 | D |
| I-18 | B |
| I-19 | B |
| I-20 | B |
| I-21 | B |
| I-22 | B |
| I-23 | B |
| I-24 | B |
| I-25 | C |
| I-26 | D |
| I-27 | B |
| I-28 | C |
| I-29 | A |
| I-30 | A |
| I-31 | A |
| I-32 | D |
| I-33 | A |
| I-34 | B |
| I-35 | A |
| I-36 | A |
| I-37 | D |
| I-38 | B |
| I-39 | B |
| I-40 | D |
| I-41 | A |
| I-42 | A |
| I-43 | A |
| I-44 | B |
| I-45 | A |
| I-46 | B |
| I-47 | A |
| I-48 | D |
| I-49 | A |
| I-50 | A |
| I-51 | D |
| I-52 | B |
| I-54 | B |
| I-55 | B |
| I-56 | B |
| I-57 | D |
| I-58 | A |
| I-59 | A |
| I-60 | A |
| I-61 | B |
| I-62 | C |
| I-63 | A |
| I-64 | B |
| I-65 | A |
| I-66 | B |
| I-67 | D |
| I-68 | B |
| I-69 | C |
| I-70 | D |
| I-71 | D |
| I-72 | B |
| I-73 | B |
| I-74 | D |
| I-75 | B |
| I-76 | D |
| I-77 | B |
| I-78 | D |
| I-79 | C |
| I-80 | D |
| I-81 | C |
| I-82 | C |
| I-83 | C |
| I-84 | B |
| I-85 | B |
| I-86 | A |
| I-87 | A |
| I-88 | A |
| I-89 | B |
| I-90 | B |
| I-91 | B |
| I-92 | A |
| I-93 | B |
| I-95 | B |
| I-96 | A |
| I-97 | B |
| I-98 | D |
| I-99 | A |
| I-100 | D |
| I-101 | B |
| I-102 | C |
| I-103 | C |
| I-104 | D |
| I-105 | B |
| I-106 | A |
| I-107 | D |
| I-108 | D |
| I-109 | D |
| I-110 | B |
| I-111 | B |
| I-112 | C |
| I-113 | B |
| I-114 | B |
| I-115 | C |
| I-116 | B |
| I-117 | B |
| I-118 | C |
| I-119 | C |
| I-120 | A |
| I-121 | A |
| I-122 | A |
| I-123 | D |
| I-124 | B |
| I-125 | A |
| I-126 | A |
| I-127 | A |
| I-128 | A |
| I-129 | A |
| I-130 | B |
| I-131 | A |
| I-132 | A |
| I-133 | A |
| I-134 | C |
| I-135 | B |
| I-136 | C |
| I-137 | D |
| I-138 | B |
| I-140 | A |
| I-141 | C |
| I-142 | A |
| I-143 | B |
| I-144 | A |
| I-145 | A |
| I-146 | A |
| I-147 | A |
| I-148 | A |
| I-149 | D |
| I-150 | C |
| I-151 | A |
| I-152 | A |
| I-153 | A |
| I-154 | A |
| I-155 | D |
| I-156 | A |
| I-157 | A |
| I-158 | A |
| I-159 | A |
| I-160 | B |
| I-161 | A |
| I-162 | B |
| I-163 | A |
| I-165 | A |
| I-166 | A |
| I-167 | A |
| I-168 | A |
| I-169 | A |
| I-170 | A |
| I-171 | B |
| I-172 | B |
| I-173 | D |
| I-174 | B |
| I-175 | C |
| I-176 | C |
| I-177 | C |
| I-178 | D |
| I-179 | A |
| I-180 | A |
| I-181 | C |
| I-182 | D |
| I-183 | D |
| I-184 | D |
| I-186 | A |
| I-187 | C |
| I-188 | A |
| I-189 | A |
| I-190 | B |
| I-191 | B |
| I-192 | B |
| I-193 | B |
| I-194 | C |
| I-195 | D |
| I-196 | B |
| I-197 | D |
| I-198 | A |
| I-199 | C |
| I-200 | D |
| I-201 | D |
| I-202 | D |
| I-203 | C |
| I-204 | C |
| I-205 | B |
| I-206 | B |
| I-207 | D |
| I-208 | C |
| I-209 | B |
| I-210 | A |
| I-211 | C |
| I-221 | C |
| I-222 | A |
| I-223 | B |
| I-224 | B |
| I-225 | D |
| I-226 | D |
| I-227 | C |
| I-228 | D |
| I-229 | C |
| I-230 | B |
| I-231 | D |
| I-232 | A |
| I-233 | C |
| I-234 | D |
| I-235 | B |
| I-236 | C |
| I-237 | B |
| I-238 | C |
| I-239 | D |
| I-240 | C |
| I-241 | C |
| I-242 | C |
| I-243 | B |
| I-244 | B |
| I-245 | B |
| I-246 | C |
| I-247 | B |
| I-248 | D |
| I-249 | C |
| I-250 | B |
| I-251 | B |
| I-252 | C |
| I-253 | D |
| I-254 | B |
| I-255 | D |
| I-256 | D |
| I-257 | D |
| I-258 | C |
| I-259 | D |
| I-260 | B |
| I-280 | A |
| I-281 | C |
| I-282 | D |
| I-283 | D |
| I-285 | A |
| I-286 | A |
| I-287 | D |
| I-288 | C |
| I-291 | B |
| I-292 | D |
| I-293 | D |
| I-294 | A |
| I-295 | C |
| I-296 | B |
| I-297 | A |
| I-298 | D |
| I-299 | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 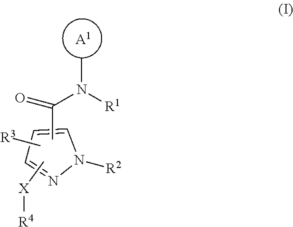
or a pharmaceutically acceptable salt thereof; wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered saturated or partially unsaturated oxo-substituted ring containing 0, 1, 2, or 3 heteroatoms independently selected from oxygen, nitrogen, and sulfur;
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²,
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸) (R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, 3, or 4 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur, 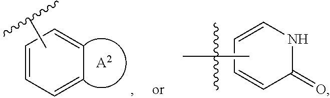
wherein the phenyl, heteroaryl, 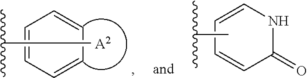
are substituted with t occurrences of R⁷;
R⁵ is —C(O)—N(R⁹)(R¹⁰) or a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R⁹)C(O)R¹¹, or —SO₂R¹¹;
R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene)-, —(C₀₋₂ alkylene)-N(R⁹)—(C₀₋₂ alkylene)-, or —C(O)—; and
m, n, q, t, and y are independently 0, 1, or 2;
provided that R² and R³ are not simultaneously C₁₋₄ alkyl.

2. The compound of claim 1, wherein the compound is a compound of Formula I.

3. The compound of claim 1 or 2, wherein R³ is C₁₋₆ haloalkyl.

4. The compound of claim 1 or 2, wherein R³ is —CF₃.

5. The compound of claim 1 or 2, wherein R³ is C₁₋₆ alkyl.

6. The compound of claim 1 or 2, wherein R³ is methyl.

7. The compound of any one of claims 1-6, wherein the compound is a compound of Formula Ia or a pharmaceutically acceptable salt thereof:

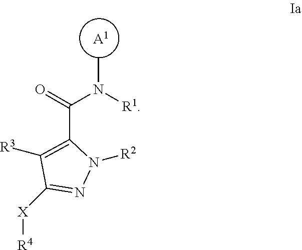

8. The compound of any one of claims 1-6, wherein the compound is a compound of Formula Ib or a pharmaceutically acceptable salt thereof:

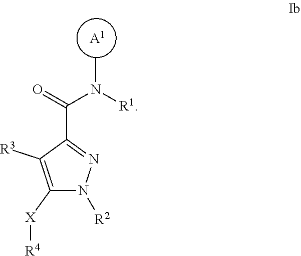

9. The compound of any one of claims 1-8, wherein R¹ is hydrogen.

10. The compound of any one of claims 1-8, wherein R¹ is C₁₋₄ alkyl.

11. The compound of any one of claims 1-10, wherein X is a bond.

12. The compound of any one of claims 1-10, wherein X is C₁₋₅ alkylene or C₃₋₅ cycloalkylene.

13. The compound of any one of claims 1-10, wherein X is C₂₋₄ alkenylene, —(C₀₋₂ alkylene)-O—(C₀₋₂ alkylene), or —(C₀₋₂ alkylene)-N(R⁹)—(C₀₋₂ alkylene).

14. The compound of any one of claims 1-13, wherein A¹ is pyridinyl substituted with m occurrences of R⁵ and n occurrences of R⁶.

15. The compound of claim 1, wherein the compound is a compound of Formula Ic or a pharmaceutically acceptable salt thereof:

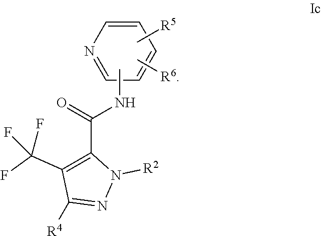

16. The compound of claim 1, wherein the compound is a compound of Formula Id or a pharmaceutically acceptable salt thereof:

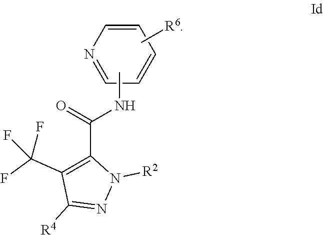

17. The compound of claim 1, wherein the compound is a compound of Formula Ie or a pharmaceutically acceptable salt thereof:

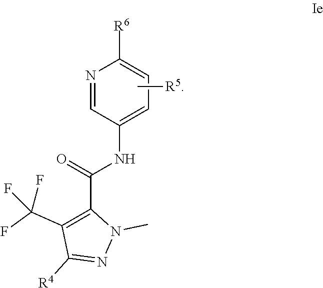

18. The compound of claim 1, wherein the compound is a compound of Formula If or a pharmaceutically acceptable salt thereof:

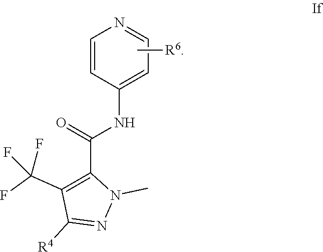

19. The compound of any one of claims 1-18, wherein R⁴ is phenyl substituted with t occurrences of R⁷.

20. The compound of any one of claims 1-18, wherein R⁴ is a 5-6 membered heteroaryl containing 1, 2, or 3 heteroatoms independently selected from oxygen, nitrogen, and sulfur, wherein the heteroaryl is substituted with t occurrences of R⁷.

21. The compound of any one of claims 1-18, wherein R⁴ is pyridinyl substituted with t occurrences of R⁷.

22. The compound of any one of claims 1-18, wherein R⁴ is a 9-10 membered bicyclic heteroaryl containing 1, 2, or 3 heteroatoms independently selected from oxygen, nitrogen, and sulfur, wherein the heteroaryl is substituted with t occurrences of R⁷.

23. The compound of any one of claims 1-18, wherein R⁴ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁷.

24. The compound of any one of claims 1-18, wherein R⁴ is 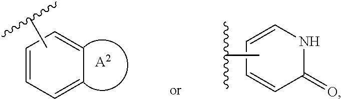
each of which are substituted with t occurrences of R⁷.

25. The compound of claim 1, wherein the compound is a compound of Formula Ig, Ih, Ii, or Ij or a pharmaceutically acceptable salt thereof:

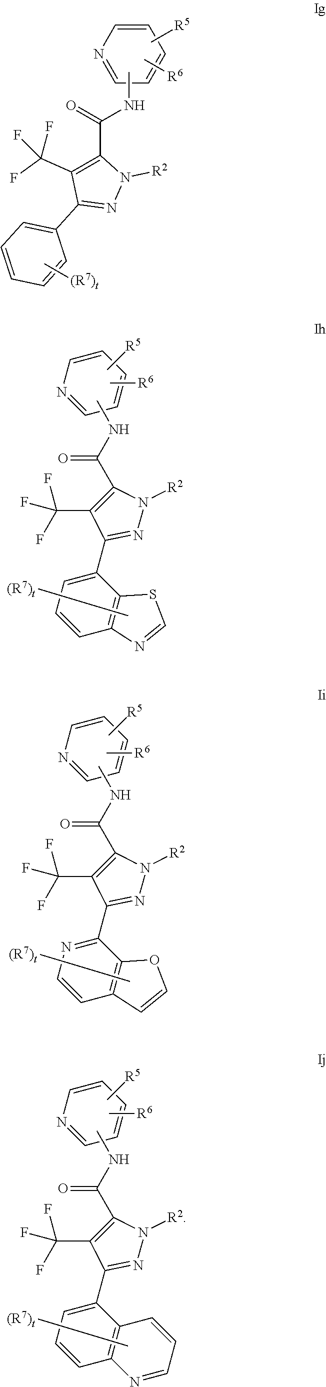

26. The compound of claim 1, wherein the compound is a compound of Formula Ik, Il, Im, or In or a pharmaceutically acceptable salt thereof:

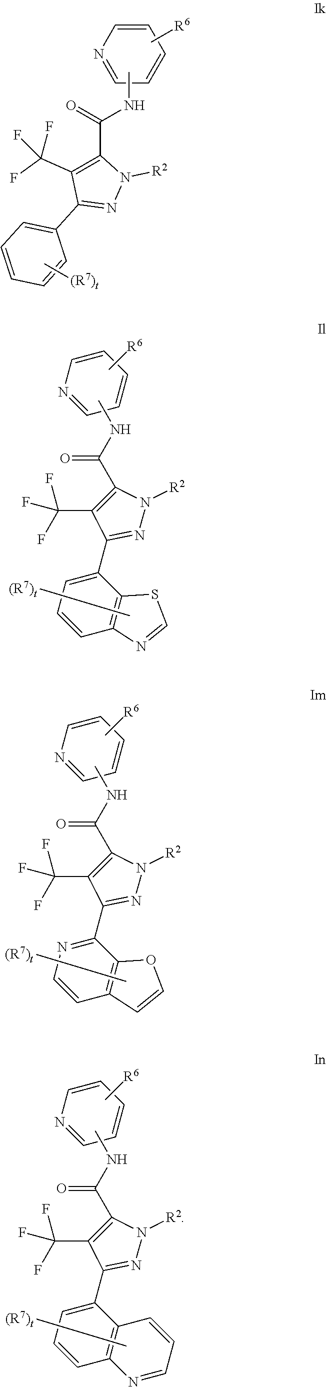

27. The compound of any one of claims 1-26, wherein R⁷ represents independently for each occurrence halo, hydroxyl, C₁₋₆ alkyl, C₁₋₆ haloalkyl, C₁₋₆ hydroxyalkyl, C₁₋₆ alkoxyl, or C₃₋₇ cycloalkyl.

28. The compound of any one of claims 1-26, wherein R⁷ represents independently for each occurrence halo, C₁₋₆ alkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, or —N(R⁹)(R¹⁰).

29. The compound of any one of claims 1-26, wherein R⁷ represents independently for each occurrence halo, C₁₋₆ alkyl, —(C₀₋₄ alkylene)-C(O)R⁸, —C(O)N(R⁹)(R¹⁰), —N(R⁹)C(O)R¹¹, —SO₂R¹¹, or —O-A³.

30. The compound of any one of claims 1-29, wherein t is 1.

31. The compound of any one of claims 1-26, wherein t is 0.

32. The compound of any one of claims 1-31, wherein R² is C₁₋₆ alkyl.

33. The compound of any one of claims 1-31, wherein R² is methyl.

34. The compound of any one of claims 1-33, wherein R⁵ is a 5 membered heteroaryl containing 1, 2, or 3 heteroatoms independently selected from oxygen, nitrogen, and sulfur, wherein the heteroaryl is substituted with q occurrences of R¹².

35. The compound of any one of claims 1-33, wherein R⁵ is a 1,2,3-triazolyl, pyrazolyl, oxazolyl, imidazolyl, isoxazolyl, pyrrolyl, or furanyl, each of which substituted with q occurrences of R¹².

36. The compound of any one of claims 1-33, wherein R⁵ is a 1,2,3-triazolyl substituted with q occurrences of R¹².

37. The compound of any one of claims 1-36, wherein q is 0.

38. The compound of any one of claims 1-37, wherein R⁶ represents independently for each occurrence halo, C₁₋₆ alkyl, or C₁₋₆ haloalkyl.

39. The compound of any one of claims 1-37, wherein R⁶ is chloro.

40. The compound of any one of claims 1-37, wherein R⁶ is C₁₋₆ haloalkyl.

41. The compound of any one of claims 1-37, wherein R⁶ is —CF₃.

42. A compound represented by Formula II: 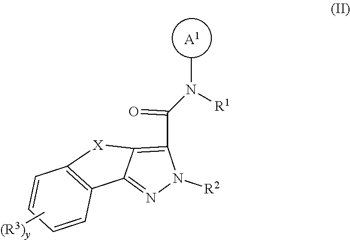
or a pharmaceutically acceptable salt thereof; wherein:
A¹ is a 6 membered heteroaryl containing 1 or 2 nitrogen atoms, wherein the heteroaryl is substituted with m occurrences of R⁵ and n occurrences of R⁶;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, or C₃₋₇ cycloalkyl;
R³ represents independently for each occurrence halo, hydroxyl, C₁₋₆ alkyl, C₁₋₆ haloalkyl, C₁₋₆ alkoxyl, or C₃₋₇ cycloalkyl;
R⁴ and R⁷ are independently hydrogen, C₁₋₆ alkyl, or C₃₋₇ cycloalkyl, or R⁴ and R⁷ are taken together with the nitrogen atom to which they are attached to form a 3-7 membered heterocyclic ring containing 1 nitrogen atom;
R⁵ is a 5-6 membered heteroaryl containing 1, 2, or 3 heteroatoms independently selected from oxygen, nitrogen, and sulfur, wherein the heteroaryl is substituted with q occurrences of R⁸;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, or —N(R⁴)(R⁷);
R⁸ represents independently for each occurrence halo, hydroxyl, C₁₋₆ alkyl, C₁₋₆ haloalkyl, C₁₋₆ alkoxyl, or C₃₋₇ cycloalkyl;
X is a C₁₋₇ bivalent straight or branched saturated hydrocarbon chain wherein 1 or 2 methylene units of the chain are independently and optionally replaced with O; and
m, n, and q are independently 0, 1, or 2.

43. The compound of claim 42, wherein the compound is a compound of Formula II.

44. The compound of claim 42 or 43, wherein A¹ is pyridinyl substituted with m occurrences of R⁵ and n occurrences of R⁶.

45. The compound of any one of claims 42-44, wherein R⁵ is a 5 membered heteroaryl containing 1, 2, or 3 heteroatoms independently selected from oxygen, nitrogen, and sulfur, wherein the heteroaryl is substituted with q occurrences of R⁸.

46. The compound of any one of claims 42-44, wherein R⁵ is 1,2,3-triazolyl, pyrazolyl, oxazolyl, imidazolyl, isoxazolyl, pyrrolyl, or furanyl, each of which substituted with q occurrences of R⁸.

47. The compound of any one of claims 42-44, wherein R⁵ is 1,2,3-triazolyl substituted with q occurrences of R⁸.

48. The compound of any one of claims 42-47, wherein q is 0.

49. The compound of any one of claims 42-48, wherein m is 1.

50. The compound of any one of claims 42-44, wherein m is 0.

51. The compound of any one of claims 42-50, wherein n is 1.

52. The compound of any one of claims 42-51, wherein R⁶ represents independently for each occurrence halo, C₁₋₆ alkyl, or C₁₋₆ haloalkyl.

53. The compound of any one of claims 42-51, wherein R⁶ is chloro.

54. The compound of any one of claims 42-51, wherein R⁶ is C₁₋₆ haloalkyl.

55. The compound of any one of claims 42-51, wherein R⁶ is —CF₃.

56. The compound of any one of claims 42-52, wherein R¹ is hydrogen

57. The compound of any one of claims 42-56, wherein R² is C₁₋₆ alkyl.

58. The compound of any one of claims 42-56, wherein R² is methyl.

59. The compound of any one of claims 42-58, wherein X is —CH₂CH₂— or —CH₂CH₂CH₂—.

60. The compound of any one of claims 42-58, wherein X is —CH₂CH₂CH₂— wherein one CH₂ is replaced with —O—.

61. A compound represented by Formula III: 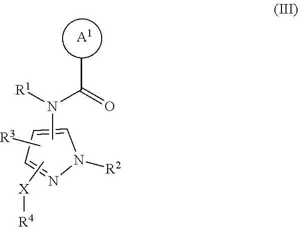
or a pharmaceutically acceptable salt thereof, wherein:
A¹ is phenyl or a 6 membered heteroaryl containing 1 or 2 nitrogen atoms, wherein the phenyl and heteroaryl are substituted with m occurrences of R⁵ and n occurrences of R⁶;
A² is a 5-7 membered partially unsaturated ring containing 0, 1, 2, or 3 heteroatoms independently selected from oxygen, nitrogen, and sulfur, or a 5-7 membered partially unsaturated oxo-substituted ring containing 0, 1, 2, or 3 heteroatoms independently selected from oxygen, nitrogen, and sulfur;
A³ represents independently for each occurrence a 4-6 membered saturated heterocyclyl containing 1 or 2 heteroatoms independently selected from oxygen, nitrogen, and sulfur, wherein the heterocyclyl is substituted with y occurrences of R¹²;
R¹ is hydrogen or C₁₋₄ alkyl;
R² is C₁₋₆ alkyl, C₁₋₆ haloalkyl, C₁₋₆ deuteroalkyl, C₁₋₆ hydroxyalkyl, C₃₋₇ cycloalkyl, —(C₁₋₆ alkylene)-N(R⁸)(R⁹), —(C₁₋₆ alkylene)-C₁₋₆ alkoxyl, or a 3-5 membered saturated heterocyclyl containing 1 or 2 heteroatoms independently selected from oxygen and nitrogen; or R² and R¹ are taken together with their intervening atoms to form a 5-7 membered ring containing 2 nitrogen atoms; or R² and one occurrence of R⁷ are taken together with their intervening atoms to form a 5-7 membered ring containing 1 nitrogen atom;
R³ is C₁₋₆ haloalkyl, C₁₋₆ alkyl, or hydrogen;
R⁴ is phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur, 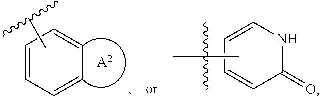
wherein the phenyl, heteroaryl, 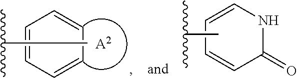
are substituted with t occurrences of R⁷;
R⁵ is a 5-6 membered heteroaryl containing 1, 2, or 3 heteroatoms independently selected from oxygen, nitrogen, and sulfur, wherein the heteroaryl is substituted with q occurrences of R¹²;
R⁶ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), —N(R)C(O)R¹¹, or —SO₂R¹¹;
R⁷ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, cyano, —O—C₃₋₇ cycloalkyl, —(C₀₋₄ alkylene)-CN, —(C₁₋₄ alkylene)-(C₁₋₆ alkoxyl), C₂₋₄ alkynyl, —N(R⁹)(R¹⁰), —(C₀₋₄ alkylene)-C(O)R⁸, —C(O)N(R⁹)(R¹⁰), —N(R⁹)C(O)R¹¹, —SO₂R¹¹, or —O-A³;
R⁸ is —OH, —O—(C₁₋₆ alkyl), —O—C₃₋₇ cycloalkyl, or A³;
R⁹ and R¹⁰ are independently hydrogen, C₁₋₆ alkyl, or C₃₋₇ cycloalkyl, or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² represents independently for each occurrence halo, hydroxyl, C₁₋₆ alkyl, C₁₋₆ haloalkyl, C₁₋₆ alkoxyl, or C₃₋₇ cycloalkyl;
X is a bond, C₁₋₅ alkylene, C₃₋₅ cycloalkylene, C₂₋₄ alkenylene, —(C₀₋₂ alkylene)-O—(C₀₋₂ alkylene), —(C₀₋₂ alkylene)-N(R⁹)—(C₀₋₂ alkylene), or —C(O)—; and
m, n, q, t, and y are independently 0, 1, or 2;
provided that R² and R³ are not simultaneously C₁₋₄ alkyl.

62. The compound of claim 61, wherein the compound is a compound of Formula III.

63. The compound of claim 61 or 62, wherein R³ is C₁₋₆ haloalkyl.

64. The compound of claim 61 or 62, wherein R³ is —CF₃.

65. The compound of claim 61 or 62, wherein R³ is C₁₋₆ alkyl.

66. The compound of claim 61 or 62, wherein R³ is methyl.

67. The compound of any one of claims 61-66, wherein A¹ is pyridinyl substituted with m occurrences of R⁵ and n occurrences of R⁶.

68. The compound of any one of claims 61-67, wherein R⁴ is phenyl substituted with t occurrences of R⁷.

69. The compound of any one of claims 61-67, wherein R⁴ is a 5-6 membered heteroaryl containing 1, 2, or 3 heteroatoms independently selected from oxygen, nitrogen, and sulfur, wherein the heteroaryl is substituted with t occurrences of R⁷.

70. The compound of any one of claims 61-67, wherein R⁴ is a 9-10 membered bicyclic heteroaryl containing 1, 2, or 3 heteroatoms independently selected from oxygen, nitrogen, and sulfur, wherein the heteroaryl is substituted with t occurrences of R⁷.

71. A compound in Table 1, 2, or 3, or a pharmaceutically acceptable salt thereof.

72. A pharmaceutical composition comprising a compound of any one of claims 1-71 and a pharmaceutically acceptable carrier.

73. A method for treating a disease or condition mediated by MALT1, comprising administering to a subject in need thereof a therapeutically effective amount of a compound of any one of claims 1-71 to treat the disease or condition.

74. The method of claim 73, wherein said disease or condition mediated by MALT1 is a proliferative disorder.

75. The method of claim 73, wherein said disease or condition mediated by MALT1 is an inflammatory disorder.

76. The method of claim 73, wherein said disease or condition mediated by MALT1 is an autoimmune disorder.

77. The method of claim 73, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

78. The method of claim 73, wherein said disease or condition mediated by MALT1 is cancer.

79. The method of claim 78, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

80. The method of claim 78, wherein the cancer is a lymphoma or leukemia.

81. The method of claim 78, wherein the cancer is a B-cell lymphoma or chornic myelocytic leukemia.

82. The method of claim 73, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

83. The method of claim 73, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenia thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythematosus, Hashimoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

84. The method of claim 73, wherein said disease or condition mediated by MALT1 is allergic rhinitis, nasal inflammation, asthma, chronic obstructive pulmonary disease (COPD), bronchitis, emphysema, chronic eosinophilic pneumonia, adult respiratory distress syndrome, sinusitis, allergic conjunctivitis, idiopathic pulmonary fibrosis, atopic dermatitis, asthma, allergic rhinitis, arthritis, inflammatory bowel disease, Crohn's disease, ulcerative colitis, multiple sclerosis, endometriosis, eczema, psoriasis, rosacea, or lupus erythematosus.

85. The method of any one of claims 73-84, wherein the subject is a human.

86. A method of inhibiting the activity of MALT1, comprising contacting a MALT1 with an effective amount of a compound of any one of claims 1-71 to inhibit the activity of said MALT1.
